SEQUENCE LISTING (383 sequences)

SEQ ID NO: 1 - DNA, 68641 bp, Homo sapiens
  misc_feature 1..68641: note = Human UBE3A-ATS genomic sequence
  source 1..68641: mol_type = genomic DNA
tgagatgacctaaacaactgtggagaatcattgatatatttccttttttcactgttcatgttgggtgaaaataatcttgtagtgaaattcacatgttctaaatattgtttttttacatctttatctggcacattcataacatagatgtttctatacatattagtactgtaatcataccatatattattctgttaccccacttactccttaaacttttagttaattaaagagtttttataaagtcccccaatagattttttttttttgagacatagtctcactctgttgcccaggctggagtgcagtggcgtgattttggctcactgcaacctccccatcctgggttcaagcaattctcctgcctcagcctgccaagtagctgggattacaggtgcctgccaccacgcccgactaatttttgtattttcagtagtgacagggtttcaccatcttggccaggctggtcttgaactcctgacctcgtgatccacccgccttggcctctcagagtgctaggattacaggcttgagccactgcacccggccagattttaatctaattttattagaacaattcagtcatatgttttttcatgctatgtatatgagagttccattattcagatactaaacaaatgtctactgtacatttactgttctcactgatgatgcattagataaccatgcacaaaataagcctggctgtggaaacgcttatttgttgggagggtgcttgtttggatcgatgatgagaataattgtctgaggatgctgagggactcattccagatgtcaatctgaggtccagatgtgcggccctccaataggacaaataagactctcagagcctggctctatttggggatccctcagtgacaacatagtacccctgtgagcgtgccttttctatctcttcgaagagggcagtggcatcctgtcttatgagtcagtgtgcactttagtgtgcctagtgacccaagacttgctttaattgtagatagatacttacatataggaaatatttcttaagtaacaaatgaaaaactttagaagattgaattaagggtcaagcaactgtgatatgtctgaaaatctcattagtgttgtgctgaaagaaggaaatatggcatgcctctattaaataatgacagtggaaccaagtttattgctttgttatttttactgtggagtattttctaagattatttttgctttttttttctttcatgttttgctgagatagaaggcctggaatctgatcctccacttcagagaacaggggtgagtagctaagccattatcttttgaaattcatatgtcatgtgctctttgctaggtctttaggtcgttttgtacatcttttcagaagcttattggaggacattttcatgatatgtccttttcctcattgagaccctcaccatgtcacctacactattgaatccttatcatttctcttttaattttaactctcttttgcttttatggaaaaatgtagaatttaagagaatttttggcaatttcatattggatcaaaatgtattgtagtgaaatccaggtgtgccaaaatattaacagattttccccatctgtttaattattggggtttcagaatagagactccatggttcataatatctttgtggtcatactacattatatttctgcttctaatttaattattaaatattgacttgaattagtcttttcctcattgttgcaacaaggtaagttatataggaaattttcttctcttgatggcatgtctgagataatcatagatataagacacctggctggtttctagaatgcatgtaatttttatttcttgatctgtgtgttgagtacttgctgtgattgcatcatgcaaatacattgagctttacaattttagtgtatgcacttttctacatgtatattattcttcgatagaaagtaaaaaaaacttatcgaactagtcaaaatattggttaatacataaaaaaagtctcaagtagattgtgtattacatggtgcttgttgattgatgccctcataatagatcaagtgggttctctctttagcacagggctttttagcaaatcatgtcatgagtagttactcaagtatttttattttaacacatttatattttttctatgtatattcttaaattctcttatacttttttctctgttataaaaacatgctgaacaatctcaagtcttaaggattgcagtattgtccccacatattcatgtattttggtactcaattctttatactttctttgacagatcacttgaactggcacatgtctcttgttttgcagagagggaattaatgtgataccttcatgcttttctattctatgtgctacataattgaatatacaagcaaatatagttgttaagatttagtgtgattatttctacaccacatgcaaagaagtttctcatagatcttaatagaggcccacatgcattgtacagtttagaatttggggaaatattgatgaagttgggtaaagtataaagccaaaagtcagaacagtgaactccttgcttaaggatttccttggagattacttagtcaatacacaactgataaatttaagtgcttttcaccttttgagttctcgacatactaaagctaaaatgtgtttcaacttttaatcctgcttccctgattttcccttttttagtctgagatcaaagagtttcagccataaattactgccaagagtaatcacttcattttaagaaagcttaacaatatagaagaatataaaattatttatgacagatgtatttttaaccttttccccatgctttccagaggaaatatgtttaatcatctgccctatattagggaaaaactttctatgctaatacaagtatctatcaatccatttatctttctatataagatgtattgatcataaccaattaactttactgtaaatgagctttagatttgacattttggtagtaatatatgttgtacaatctcctgaggtcctataggtcttgaggtctctatgtcaaaaactatagatgtgccagtgtcctcagtgaatgtgaaggacaccacattttccttagccatttcttgttttcagaataatggttatcaacattttgctactgcaagataccatacatttataatcggaatatgccagtttttatgcactcatgcctctgtttctgtagagcattcccagaatgagtaatgcttgaaaattaggtccatgtgatttctttaatgagttatagtcaaatcatgaaatattcaggttaccattatttcagtaatgtattagaatgtcaaggtaaagttatctacattgtatatatacacacaacatagatataatttatacaacatctatatttatacaacagatatatatttatgtatatatttatacaacataaaatatattttattatttaaacataaaatatattttattatttaatatagattcttaagtgataaatatgtttaatattattaaaataggttaaaataggttatagttagtacagtgaaaattggcagcccttttacaaaacatatgccataactatagcatttatcactgacagtcataccaggatagtcttttatttccaatcacttaaatattcctaattgcaaaagaaatttgaagactaaaattcagaagttttgaaagagccattgcctgggtaaactatacaggtttcagttttatttataataattatgaggccaggcgcagtggctcacacctgtaatcccaacactttgggaggccgaggcgggtggatcacgaggtcaggagatcgagaccatcctggctaacacggtgaaaccccatctctactaaaaatacaaaaaattagccgggcgtggtggcgggcgcctgtagtcccagctactcgggaggctgaggcaggagaatggcgtgaacctggtaggcggagcttgcagtgagccgagatcgcgccactgccctccagcctgggagacagtgcgagactccgtctcaaaaaaaaaaattatgtatatatttataaattaatacttaataaattaataacttgtgataggcaatgcaaagatgacagtaaaaggacaaaattgattagattgataaagtcctgttaacatgaagaaattgaccaggataccatccacactataaagttagagaaatttatcaggacaattccctaaaatactcttctcaattttaacattgtaacaggaatttttaaaattttggtattatgtgtgtttccttccagataatttgaacagattcatatttggtatttttaaaagccatatctttgtccttagtgctggcaatgtattcttgagaatgaacaaataagagatacgtaaaagcataagagaaggtatcaggttgaagtagtcaatcagttatacagaacacaaagaattttatcttgtataatgtttatatagctttatagaagtgtgctgaaagggctataaaacatggacattattatctcattgaaaggtccaatacgtactgaaatacatgcttttattttgaaccaaccaccctataaacgttgtatggcttatttagatgagagcccaggttgtgtgtgtctgtgtgtgtgtgtgtgtgtgtgtgtgtgtgtgtgtgtgtgtgtgtacctgacagggaagcaagaacatcgagttgccaatgcactctgtctatggttagaatcatgctgaaaacatggctcccccagttctggaatgagcccacagatcaagcattccccaaagacatagcaggctcaaatccctgtgtacacaatattttatgattatcttatgtcagtactttcaaagtatacagtttgtgtgaagacaaatccaatgtcatttttcttggctagcctatatgtgtggtaaatccattatttacttacttgcttcctgaaaattacaattagattaacaaactgcagcaaagtgggcatgatgagatagagattgaagtgtaagcttatgttaatgatgcccttggtttggataaacacatctaagagaaaaatggaaaaacacacatggcagggaagccttgatagagccaaaatataggattgtatgtagtaatgcaatccatagatgagcatttggcagtaatattatttttcagatatggataaaaattgcttaggagagtaaagagagacaaagttgaaagcaggtttatagtaggtgttgttttagtgttgatccctttttgctccaataatcaaagtgataaatattgaaaattgattcatgcagcattacttactccattctaatttttatatatgtcaaaagtgccatctcccaaactgtgctatccccttcaggagaagagactctgctgaagtttataaggttgacatattgccagcttcaataatgtaaagatgaagtgtatactgaattcttaatgcaaataacaactctattggaaagtaacccagttatagaagtgctaatttgtcaggagctgccttaccaagatcatgatgagtacagttatctcaggattctgaaagattgttttccgatttcaactagtctagctgaatgttccttgatagaaagagaggacttttagaattggttcaatatgatgacctcctgaattatctcacatagcccgtttgtacatgcctttcttttctctcagaaaatggcactatcataatagctttcttacacagacttcaccttagggttttacattaagggaggggtctggtgtttcatttattttgaagtatttgttgttgattgtgtacagtgcttgagtaaaaaattgaatatagaaacatctagaatatttttttaaaggatcagtgtttataaagtgaattattagtgtcaataatgttgggaaagttttaagagaatataggaaacttgaacattacacaactacaatgggaccaaattgtggggtctcattatagttaatatttatgtatttttttccaattgatttgtgtgctttttttctgcatgtttttggcagatagaatggctataacaagtaacagcatgtcaggtaataaaaataagcagagccctattcctttaaaaatcttcactgatgggagggccataaaataagtcttaatacatttaaagaattaaattcatgtaaaccatgttaatttaattccacaatgatattgaattagaaataagaggaatatctcttgaacatctcctaaatgtttggaaatttaaattagcatttctgacctatttattggttaaaaaagatacaaagaaaggaaaattgaaaagtcttttgaactgaataaaaataaaaatatagaatctaaaactttatgggatactgacaaaacaggatatagggaataatttatagcactgaaatgcctatattagaaaagaaaaaaggttttaaatcagtaaatttgtattttaccttaagaaacttagaaaagaacaaattaacccagacttaagtaaaataaaggcactaataaagataagagcagaaatcaatgaaatataaaacaacaaaacacagagaaaaattgagaaaatttaaaaatagcctagtgagaagatattgataaacttgtaaccagaccaatttaagaaaaaaagtcaaaacacaaataccaatatttgaaaatgtaggagggcaaatcattacagattctatgaatactaaaatgataataaggaaaaattatttaaaaggggcatgtcagccaggcatggtggcttacccctgtaatcccagcactttggcaggccgaggtgggaggattgctagagctcaggcattcgagaccagcctgggcaacatgttgaaaccttgtctacacaaaaagtacaaaaattagctgggtgtggtgttgcacacttgtagtcccagtcacttgggaggctgaggcgagaggatcacttgagcccaggaggttgaggctgcagtgagccatgtttgtaccactgccctccagcctgggtgacaaagtaagaccctatgtaaaaaaaaaaaaatgtatgccaacatttttcaataacttaaatgaaatggaaaaattccttgaaagacacgaactacaaaaactcagtgaacaagtaaataacctgaatagccctgtatcaagtaaattgaatttgtagttaaaagccttccaacagagaaaacttcaggtacctatagcttcatatgaaatgaaaaaaaaaataccaatcctctacaagattccagaacatttaaaagaagggaatatttcccaacttattccatttggacagcaatacccaggtaagaaaaagagacacagaaatttaaaaagaagaatatacattattccttaggaacataaatgcaaagaaatctaatcaaaattttggcaaatgaaatgtagaaatactttatgaccaagtgagagttacccaaagaatttaaggttggttttatatgtaaagatcaaccaatataggaaaatcacttctggaaagtcagagtaagaaactccaaaaatctactcctccataaaaccaataacagccttgatagaaatagttgaaattaattttccaaaactttggaaattaaccaaaggcttacaaaattccagagaacattaattcaagaaaaatggctgaatcagtaagaacagccagctttgtggcattttaatatgaccccttcccatgcttttctccctagtgctgagatagtcttaaaaattagcaggatagcaaccactggagaagaaaggtttggaaatttcccaaaaagttccatccccatagaattatcactatttgacctctaaagcccaatctataggatttatattcatttggactgactcagagctcactcagtaaggaaatctcaatttcaaggtattggtcaaaaagaatccatggcaattgttgactatcacaactgcctgaagtcttggtaacagttgggataaacaagaagctgatcaaaaactgaaaactaaaatcttgggaatgagatatctacaggatgcttcaaaaagctttgatacattcctgtttatctagaaagctacatgcaggctgggtgcagtggctcacacctgtaatcccagcactttgggaggccgaggcgtgcggatcatgaggtcaggagtttgaggccagcctgaccaacatggtctctactaaaaacacaaaaattagccaggcgtggtggcgtgcatctgtaatcctagctactcaggaggctgagacaggagaatcgcttgaacccgggaggcggaggttgcagtgagccaagattgtgccactgcactccagcctgggcgacagagcaaggctctgtctcaaaaaaaaaaaaaaaaaaagccacatgcatgtaattgtttacctctggctttccttttatgctctgggcaagctaaggaagagttgtgaactacctaagtgctgaatgggaaccataacacacacacacacacacacacacacacacacagcaccttagtaaagggtgagaggcatgttagttagaagcattaaaggaaatctctttctagtcattatctgtgcactaacctaactgagcagagacttcagtatccacatactacagggcatataaactttacagaattagtccaggaaaatcatatctaaaaaaaaaaaaaagcagtaacaaaaataaactctgggaaaggggagaatatgatttaaagagttgccacattatacataatatgtctagtgttcaacaaaaaattacgagacatgcaaagaaatagaaaaatatggcacaaagaggataagatgaagtcagtgaaactatcctcgaggaagaccagatgttggtcttactagacacagacattgaaccagctattaaaaatacgtacacagaactaagaaaaacatgtcaaaagggttaaagaaaggtataaaaatagtgtcttaccaaatatagactaccaataaagagatagaaattataagaaaagacaacatgaaaaaatataaagcaaaaaaattagacaattgaaacaagagggcctctattcgcagatttgagcaggcagaagaaagaatcagtgaacttgaagatatgtcaactgagattatccagtctgagcaacaaaggtgggaaaaaatgaagaaaactgagcaacaaagaactgtagaacagcatctctcataccaatggatacataaactggagccctagaaggatgaaaaaaggagaaggaaagaaaacttcccaaattttaagaaaaacattaatttatatacccgagatgaccaataaaatccaattaagataatctcaaagagaccaacacctatacacatcatagtcagcgtgtcaaaagacaaacataaggagagaattcttgaagatagtaagaaaaaaattattcataacatacacaccatcctcaataagtctgacaattgacttctcactgtaaaccatgcaggctaaaaaggcaatgtacataaccaaagtgatgaaataaaaaccttcaaccaatcattctagatccaacaaaactattgttcaaaaagaagaaatgaagacattcctaaacaaaatctcagagaaatgttctctataagacttgtcctaatagaaatgctaaaggaactccttcggtctgaaatagaaaggcactggagagtaaatcaaatccacgagaagaaataaagagaaccagtataagtaactacatgtgtaagtttaaaacaaagtataaatttattttgtttgtaacatttgtcttttcctatttgatttaaaatataatctcaattataaacttgtgttgatggtattatataaagatgtaattttgggttatagcaccaaaatggcagaataggaattttctgtaggtgtttcccacataagtatcaattttgacaaccatccatgggcaagagtaccttgtgggagttcaggagttgacagtaaaacttcagcacaccagaggagtaaagaaatctaagaatagattcattggaaagggtataaacagtttcactttacctgcatcaccaacccccaaaagtggcacagctcagtaacaagagcccattatttcttccacagaggaaaaggagagtataataagtaagtgtccagtttctcaagacatacaggcccctgcccaagagatccactttattttcatctcacccagaatattgaggtgatcagcaaggtggagtggttgggagagggtaaaagcaggaaagagagatggggactcaaacagcagcccatacttggaactgccatagatcctaccagttacttcatggactccatcaggaacccacctataagccacaggggatgcatccctcccatcttgccaacaaaaccccgaatgctccaaatgcctcacccactctttggctggctcccaagtgcgctcctgtgaaaagcgagtgagtatctctgcagatggcttgcaagcacatgttgacagctggctccactctgtagaactggaaaaaagctcacatacatgagaatttcaggacactaccctaaaaaaaaaaatgagacttcagcacctggcctggctttatgcaacctagagaaggtgatatgatttggctctgtgtccccacgtaaatctcatgtcaaattgtaatccccacgtgttggacaagggacttggtgggaggtgattgaatcatgcgggtggacttccctcttgctgttcttgtgatagagttgttatgagatccagttatttgaaagtatgtagcatgtcccccttcactctcttgcactcctgctccaccttggtaagacttgcttgctacccctttgccttctgccatgattgtaagtttcctgaggcctcccagctatgcttcctgtatggcctgcagaactgaactgtgagccaattaaacctcttttcttcataaattatccagtcttaggtagttctttatagcagtgtgagaatggactaatgcagaaggcatacaacctttagaatttgcccccttgagggaacaagatgtgtgaagcaggttcatccatagaaaatgtctgagagaacctcaaaatccctaacctgactaactgatgaaagtgtttctctcctaaggccagtcagtaaagaccagagggggtgactgtttctttaaatgcaaaggcagcagcacaataattcaagaaacatgaaaaatcaagaaaacatgacaccaccagaagaacacaatcattttccaataaccaactccccaaaaatggagatttacaaattggtttataatgaattcagaacaattatgttaaggaagctcagcaaactaaaaggaacaccaatagactactctgtgaagtcaggcaaacaattcatgaacaaaactagaaattcaaaaaagagaaaaattatcttaaaagaaaacccagaagttatggagctaaagaatacaatgcatgaaatgaaggagcgtatcaacagcaaagttgatcaagcataagaaaaaaaaaatctgtgaaactgaagactggctatttgaaattattcatcagaggattaaaaaaaaaagaatgaaaagaaataaagaaagcctacaggatgtataaaacaccatcaagagaactaatataaggattattggagtcataaaggagaagagagaaaagggtagaaaacttatttaagaaataatggctgaaaactctccaaatctaggaaaagatatgagcatccaggtatatgaagctcaaagatccccgtacaggatacattccaaaaagacttcaccaaaacacatgataatcaaactgtcaaaagcaaaatcaagacgatgaataaaccaccaatcactaagagggagacaggattcttatgttgtcattattatttacatataatttcagtaaatgttattggaaaatttataatgttttaaaaaaagaaatttgaaagcaccgaaagaaaagagactcatcacatacagggaacccttttaaggcattcaagagatttctcagtagaaaccttacaaaataggagagagtgggatgaactatacaagtgctgcaaggaaaaaaatgccaaccaacgctttacctggcaaatctgttcctcagaaatgaaggagagagaagaactttcctagacaaacaaaagctgaggcagttcatcaccactagacctgccttacaagacatactaaggggagttcttcaagctgaaatgatatggcaatagttagtaatatgaaatgataaacctcactggtaaaggaaagtacatagtcaaatttagaacactttgatactataatgatggtgtataaataattttactgtgctatgaaggttaaaagacaaaagtattaaaaaaaacccatagctgcaatagcttgtcaatgcatactacagtataaaaagatgtaaattagaacattaaaaacatagcatgcaagggtagggaagtaaaagtgtagttttcatatgtaatcaaatttaatttgttatcagcttaaaataaattgttatacctatgttttatgtaagtgtcatggtaactataaaggaaaaacctctagtagatacacaaaagaaaaagagaaaggaatcaaaacataacactacagaaaattatcaaattacaaaggaagacagcaagggaggaacaaagtaaaaagagcaagaaaaaatttaacataatgaaaacagtaagtccttacgtgtcaataattactttaaatgtaaatggattaaattatccaaacaaaaaaacagactggacaaatggattttagaaacaacaacaacaacaaacaccgcacacacacacacacacacacaaaccacccagccccaactatgtgctgcctacaagagatttacttccactttaaggacacatacaggctgaaattaaaagaacagaaaaagatattgcatgcagatagaaaccagaagagaggagaggcatctatacttacagcatacagaaaagattttaagttaaaaactatatcaaaaggctaagaaggtcaaaatggtgaagcagttaattgttcaagaagacataaaaattgtaaatatttatacacccaatattgaagcacctaaatatataaggcaaatattaatacatataaaaggagaaatatacagcaatacagtaatagtagtgaacttcagtgcctccctttcaaaaatggataatccagacataaaatcaataaggaaacatttaacttaaacttcactttagaccaaatggatctaacagacattatactgaacatttcatccaacagtggtagaattcacattcttctcaagcacacatggaacattctccaggatagattatatgttagctcacaaaataatattacaaaatttaataaagctgaaatatcaattattttgcaccacaatagtataacactagaaatcaataacaagatggaaactggaaatttacaaatatatagcattaacatattcctgaacaaccaatgggtcaaagaaaaaaatcaaaataattttgtgacagcaaagtagaaacacaacataccaaaacatacaggacacagcaaaagcagttctatgaggtaagcttatattgataaacacatttaaaaaaagattttaaataaacaacattacacctcaaggaactacaaggaagaaaaaaaaacaagccccatgttatcaaagggaaggaactaacaaagatcagacagaaataaatgaaacatagactagaaaaacaatagagactattaataaaacttagagttagttttttaaaaaataaaatcaacaaacctttagctagactaaaaaaagagaagactcaaataaaataaaaaatgaaagaggagacattacaactgataccacagacatacaaattaagagaaaactatatgccaacatattagttaacttgcaatgggtaaatccctagaaacatacaacctacaaaaactgaatcatgaagaaatggaagatctgaacagatcaataatgaataagggaattgaatcaatattcaaaaatctcacaaaaagaaaagctcaggatcagatggcttcactggtgaagactgccaaccatttaaaaaaattaataccactctttattaagctcttccaaaaaaattgaagaggagaaaacactttcaaattcattataagaggccagttttaccttgatatcaaagatttaaaaagaacactttgagaaaggaaaattacaggccaaaacccttgataaatatagatgcaaaaatgctcagcaaaatactagcaaacctaattcagcaacacattataatggcatacatcatgaccaagtgagattcatgcctcggatgcaggatagttcaatataatcaaatcaacaaatgttacactactttaacagaatgaaggataaaaatcatatgatcatctcgatggttgaactagtttacagtcccaccaacagtgtaaaaatgttcctatttctccacatcctctgcagcacctgttgtttcctaactttttacagatcaccattctaactggtgtgagatggtatcttattgtggttttgatttgcatttctctgatggccagtgatggtgagcatttttcaagtgtctgttggctgcataaatgtcttcttttgagacgtgtctgttcatatccttcacctactttttgatggggttgtttgtttttttcttgtaaatttgtttgagttctttgtagattctggatattagccctttgtcagatgagtagattgcaaaaattttctcccattctgtaggttgtctgttcactctgatggtagtttcttttgctgtgcagaagctctttagtttaattagaccccatttgtcaattttgtcttttgttgccattgcttttggtgttttagacatgaagacagtgtggtgattcctcaaggatctagaactagaaataccatttgacccagccatcctgttactgggtatatacccagaggattataaatcacgctgctataagccataaaaaatgatgagttcatgtcctttgtagggacatggatgaagctggaaaccatcattctcagcaaactatcacaaggacaaaaaaccaaacaccgcatgttctcactcataggtgggaattgaacaatgagaacacatggacccaggaaggggaacatcacacactggggatggttgtggggtggggggaggggagagggatagcattaggagatatacctaatgctaaatgacgagttaatgggtgcagcacaccaacacggcacatgtgcacatatgtaacaaacctgcacgttgtgcacatgtaccctaaaacttaaagtataattaaaaaaaataatgctgctataaagacacgtgcacacgtatgttcattgcggcactattcacaatagcaaagacttggaaccaacccaaatgtccatcaatgatagactggattaagaaaatgtggcacatatacaccatggaatactatgcagccataaaaaagatgagttcatgtcctttgtagggacatggatgaagctggaaaccatcattctcagcaaactatcacaaggacaaaaaaccaaacactgcatgttctcactcataggtgggaattgaacaatgagaacacttggacacaggaaggggaacatcacacactggggcctgtcgtggggtcagggtagggggagggatagcattaggagatatacctaatgtaaatgacgagttaatgggtgcagcacaccaacacagcacatgtgtgcacatgtaccctagaacttaaagtataataaaaaataaatcatatcatcatctcagtagatttagaaaagcatttaacaatattcaacatcctttcagaactaaaaactctcaataaatcaggtatagaaagaatgtgcctcaacactataaaagccacatatgacaaacctggaggtaatatactcaatggtgaaaagtaaaaagctttgactctaagatcagaaccaaaacaaggatgtccattctcaccacttatatttaacatagtagttgaaattctagctagagcaattaggcaagaaaaaaggcacccaagttggaaagaatgaagttaaattgtctctgtagatgacatgatcttatatatagaaaacactaaagactccaccaaaatgctgttttaattagagcttaaaaaaataattcactaaagttgcaggatacaaaatcagtatacaaaaatcagttgcatttctaaacaccaaaaacaagttatccaaaaaattaagaaaacaatcctatttgtgatatcatcaaaaaataaaatactaaagaataccaaagaaactgaaaataaatggaaataaatggaaagatagcccatgttcatggattagatgaattaatactgttaaaatgttcatactacccaaagcaacctacagattaagtgcaattcctactaaaattccaatgacatttttcacagaaatagaaaacacactcctaaagtttgcatggaaccgcaaaagactcaaatagatgaagcaattttgagcaagaacagtaaagctggagacatcacactacctaacttcaaaattttattaatcaaaacagcatgacataaaaacagacagaaaagaccaatggaacagaatagagagcccagaaataaactcacgtttatagagtcaactaatattcaacgaaggtgccaagagtacacaatggggaaagtatagtctctacaataaacagcactgggaagacaatatccaaatgcaaaagaatgaaattacacccttatcttataccatacacacaaatcaaatcaaaattgataaagacttaaatataagacctgaaaccatacaatctttaggcaaaaactcattgacattggtcttggcaatgatttttttgatatgacaccagaagcacaggcaacaaaagcaaacctaaacaagtgggaccctaacaaactaaaaagtttctgcacagcaaaggaaacaatcaatcatcagaattaaaaggcaatttatgaaatgggagaaaatgtttgcaaaccacatatctgataaagggttaatatccaaaaatatataaggaatgcataaaattcaatagaaataaacaaacaaataatccaattttaaaatgggtgaagaacctgaatagacattttttcaaagaagacttacagataggcaacaggcatatgaaaaaatgcttgacatcactaataatcagggaaatgcaaatcaaagctccagtgaaataccacctccaactattaggatggctattatcaaaaactcaaaagaaaacaagctgggggaatgtagagaaaagggaaaagaagatccttatacactgttggtgtgaatttaaactggaatagcccttatggaaaacagcatggaggttcctcaaaaaattaaaaatagaactactatatgatccagcaatttcactattgagtatatatccaaatgaattaaaatcactgtcttgaagaggtatttgcacactcatatttatttcagcattattcacaatagccaagacatggaatcaacctaagtgttcatcagtagatgattagataaagagaacgtggtatatagacacagtggaatctatttagtgttcaaaaagaaggaaatccaacttttaaaatcctttaaaaagttaaactcataaaaacagagaggagaatggcggtttccaggaactggagggtgggagaatggggagatgttggtcaaaaggtacaaactttcagttataaaatgaataagttctagagatctagtgtacaacagcattactatagttaataataatattttttatacttgaaatttgctaagagtaaatatcaatattctcaatacacacaaaacagaactatctgaaggcactgatatgttaattatcttcattatagtaatcatttcacaatgtataatgaatatcaaaacaatagtgtacatcttaaatatatacagttttgatttgttaatcatacatcaatgaagctagaaaaaatgttgtaatttttaaaacaatagtaatataaattaggggtgaaggaattgacctatgttggagaaaagtttttgtaaactatttaaattaattggtatccattcaagctagattatttttaattgttaatttaattgtaatactaaggcaaccactaaaaaagcctttaaaaaaatatagcacttgaggctgggcatggtggccctcaattatatatataatatatataaaatatatattatatatatagtagatgaacaacaatgggatttaaatggtacactagaaaatatctgtttaacaaaaagaaagcaatagtggagaaatataagaacaaaaccatgtaagatttatagaaaatgaatagcaaattggttgacctaaaccctatcttataattatattaaagataaatgaaataaatactacatcaaaaggcagagattatcagaatagagaagaaaaatccaaaccataattcaaccttatgttatctgtatttagaatatttagagtcaagacacaaatagatagagttccatttgggcgtgaaaatatttaccatgcaaaatgtaattaaaatagagctagagcagcaatactaatatctgacaaaatatactttaacaaaaattgttgctaaagacaaaaaagaacattttataataagacacaacaattataaacatatacaccaaacaatagagcccaaaatatacaaagcaaaaactgctagaattgaagaaagatagaaaaatcaatgattacagttggaggtgtcaataccaaactttcagtaatacacagaacaacaagtcaaaagcaaaaaggaaataaaaaactacacattatcatacaacaccttaatacgatatccaacaatagcagaatacacattattctgaagtgcacatggaatattcttcaggatgacacatattaggctgtaaaacatatcttaataatttaaaagaattgaaataatacagcacatattctctgactgcaaaatattaaaccaattacagaagaaaatgtgggaaattcacaatgatatggagatttaaaaatacttcaaagtaaccaatgaatcaaagaacaaatcacaagagaaattagaaaatattttagatgaataaaactgaagaaacaacataccaaaattttgtggttgtagctaaagcagtgcttcaagggaaatgtataacagtatatccctaaattttaaaaaaatctcaatccaataacctaatttttcaccttaagaaatgagagtgaaagagcagacttaacccaaagtaaacagaaggaagaaataataaagattagcatggagataagtagtgaaaataaaaacaatagagaaaattaataaacttgaaagttggttcttctgatatatcagtaaaattgacgtattattagtttgaccgagaaagaacaaaagagagaagattcaagttactagacataaatgaaagtatagatatcaccttacagaaattaaaagaattctaacagaatattgtgaaaaagtgaatgtcaaaaaattaaattatatgagatacacaaatccctctaaaggcacaaactaccacagctggtgtaaaaacatgaatacaccatttacagttaaaaagactgaatagataaaaattttaagaagaaattgagtaagtaatttaattttcaaactataatcccaagaccagatgttttcattggtgaattctaccaaactttaaaaggattaatatctatttttcatacactctttccagaaaatagaaaaggagggaacactctataactcactgtatgaggtccgtattacccttatatcaagaccaaacatcataagaaaagaagactaaagacttatggtttctgctctgatatgtttggaagtcatcactactattgtcacaaggaaaaatctgaacaaactaaagtcaacgatttcttaaactaaccatagaattgaggtaacgggcgaaaactggagatgtaggcaaatacaaaaaaaatcacagtttatcaggagcagaaactgctgaaaccagcaactcgtatgaacatgttaagtggtaattgacaaatttctggagattgaatgtggactggattgagagttaggaactcctaagtgcccagtttttgatgaccccacacacttttgtaaactagacttccaagagccccagcaagtttcttacagtgaagactgcagaaaaatcccctgatgcttcagataggaggaagggaaaagcaactattttgaaataagcccaggggacaagtagttatttctaaacactctcagagcattttctttcacacggcagggggctccctgcaagggaagctactttgcctgagccttgtctgatgtaggagaaaaggaattggatggctcaagctccatctagcctttctgatttatataagggaagcaaaaaataggttaagaaactcttctgaaagtcacaactcagatttattttatatttatttatttatttatattttttgagacggagtctcgctctgttgcccaggctggagtgcagtggcgcgatctcggctcactgcaagctccacctcccgggttcacgccattcttctgcctcagcctcctgagtagctgggactacaggcgcccagctaattttttgtatttttagtagagacggggtttcaccacgttagccaggatggtctcgatctcctgacctcgtgatccacctgcctctgcctcccaaagtgctgggattacaggcgtgagccaccgcacctggcctcacaactcagatttaaccataagattatagaacacttctcctcccaaaacagagatatagatcaatggaacagaacagagccctcagaaataacgccgcatatctacaactatctgatctttgacaaacccgagaaaaacaagcaatggggaaaggattccctatttaataaatggtgctgggaaaactggctagccatatgtaaaaagctgaaactggatcccttccttacaccttacacaaaaattaattcaagatggattaaagacttaaacgttaggcctaaaaccataaaaaccctagaagaaaacctaggcattaccattcaggacataggcatgggcaaggacttcatgtctaaaacaccaaaagcaatggcaacaaaagacaaaattgacaaacgggatctcattaaactaaagagcttctgcacagcaaaagaaactaccatcagagtgaacaggcaacctacaaaattttcacaacctactcatctgacaaagggctaatatccagaatctacaatgaactcagacaaatttacaagaaaaaaacaaacaacctcatcaaaaagtgggcaaaggatataagcagacacttctcaaaagaagacatttatgcagccaacagacacatgaaaaaatgctcatcatcactggccgtcagagaaatgcaaatcaaaaccacaatgagataccatctcacaccagttagaatggcgatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattgctatatatatatatatatacccaaaggactataaatcatgctgctataaagacatatgcacaggtatgtttattgcagcactattcacaatagcaaagacttggaaccaacccaaatgtccaacaatgatagactggattaagaaaatgtggcacatatacaccatggaatactatgcagccataaaaaagatgagttcatgtcttttgtagggacatggatgaaattggaaatcatcattctcagtaaactatcgcaaggacaaaaaaccaaacactgcatgttctcactcatagatgggaattgaacaatgagaacacatggacacaggaaggggaacatcacactctggggactgttgtggggtcgggggaggggggagggatagcattaggagatatacctaatggtaaatgacgagttaatgggtgtagcacaccagcatggcacatgtatacatatgtaacaaacctgcacattgtgcacatgtaccctaaaacttaaagtataataataataaaaaaaggctacctaaaaaaaaaaaaaagaacacttctcctcccaacaccatatcaccacatcaaccaggactccagtgtaatagcagtgaattctaactgaaagaggtgaaagacactgattgtatttaagaaagatcttctaaggaaatccaaaaatagtaggggagatcaaaacaaagatactagaggaaattgaatatgtgacacctatagctacaaaaaaattaaacataacatagccctaaccatataaacataaaacctcacacaaagacctattatctgagattctgttgcctgatacattgcgttttatttcaataaaaaaattagagggtatgttaaaaagcaggaaaagttagtctaaagagacaaattgagcctcagaagtaggctcagatatggcagagatttggcaattatacctagagtttaatataaatgattaatataataagtgttctaacagaaaaaggcaacatgcaagaacggatgggtaatgtgatcagcaagaaggaaactctaagaaagaagtcaaaaggaaatgctaggaataaaaacctacaagaaataaagaatgcctgtgatgggttcctcagtagactggacaaggtcaaagaatcagtggatttgaaaatatgtcaacaaaaactgccccactgaaatacaaaagaaaaatagaattttaaaaacgtaacacaatctccaaaacagtgggacaattacaaaagatgtaatgtgcctaatgcaaatgacagtaggagtataaagggagaaaggaatagaaaatctgaagtaataatggcagagagttttccaaaattaatgctaaaccacagatacagcaagcccagagaacaacaaggaggaaatttagtaaagcgtctgcaaccaagtatgtcatattcagactgacaaaaccaaaggtgaagagaaaatattgaaagaagacaaagaggaaaataaatatcaagaaaatacatacgaaatacatcatacatacataagaaatacatcagaccatacaagcaagaagagaatggagtgaaatgtttaaaatgttgaaaaaaaaactatcaatttgcaattctgtatccagtgagattatctttcaaaagtgaagagggaaatggcagagaagtcatctccaagacctatggtttcccttcacagaaacactgaaaaatatgaacaaaagtggtcagaattaactttctaagaattctataaaatggtaaaatgtttacaccagtaaagcaaatgctgaattgagaaggcaacttaaaaaggtgaagaaaacttcgtattatttttatgtgtccttgccccacgtccttccctaccttagtcttgaagatggcagcccacatttctactgtggggctctggtttctgtttcctggttcaagagggagaataacagaccttacttttagtcattattatttccttctttctgatttccttgggtttattttgctcttctttctactttattgaaatgagaactaagattatgatttgagacatttttctaatgtaagcatttagtgctataaatttccatctcaacactgctttagtcacatcccacaaatttttatatgttgtaatttcactttcatttagttctatttttaaattttttctttttatacttcctctgactcacagattacttagaattgtgttgttcagttttcaaggatattgtagattttcctgtttctctgttgtctaatagttctgttccattttgtacagatagctcacgctgtatgatttcaattttttaaaaaattgtgctttgttttatggcccagatatggtctgtgctgtgaatattccatgttattataaagtatgcctgttatattattatatatatataatatatataattataaagcatgcccgttttgtattgttagcagagtattctagaaatgtcaatgagatcttgttggttgatggtgcttttcagttctatatctttgctaattttttttttttttgcttagtagctatatgagattctgagagaggaaattgaagtctccaaccataattgtggatttgtctatttctcctatcagttctatcagtttgtgcatcacatatttgaggctctgttgtttggtgcatacacaagtggaatcattgtgccctcttggtggcttattttatgattatatagtgcctatctttgtggtatttttatttgctcttaaatctactttgtgttatattcatatacccattcttttttaaaaaaaattgtttgcgtgatacatcttttccattcttttaatctcagcctatctgtgccattgaatttgaagtgagttttcatatagagaacatattattgaatcatcattttaaaaattccttttgccaatcttttttatactgaggtaaaattgacataaaatttatcattttaaagtgtacaatacagtggcatttggtaatacacatgttatgcaacgttaactctacctggctcctaaatgttttcatcatccccaaaaggaaacttcatactcattaagcagttaattcccattccttctctcggccactggcatccgcaaacctacttttctgtctctatgaatttacctattatggatattttgtataaattgaattatacaataagtgacctttatgtttggcttcgttcgcttcgcatactatttttcgatattcaaccatgttgtagtatgtatcagttttatttgaataactcaattctttttgttgtatagctaaaagttgattcctaggtcataatgataattctatgtttagtttattgaatagctgccaaagtttttccacagtggctctgtcattttaaaatcccactagcaatggatgagagttccaatatctccacatccttaccaatattgttattttatatttttataattataattttcctagtgaatacgcaatggtatctcattgtgtttttggtttgcctttccctaatgactaatgatgttgagcatcttacaatgtacttgttaactatttgtgttctttagagaaatgtctattcaagtgccttgtccattttaaaaatcgagttgtcttgttgacttatgagttctttaatacagtaaacgcttatcagatatgatttataagtattttaacccattctgaaggtcaccttttcacttttgtggtagaccattatgcacaaaggttttaattttgataaatccaatttatccgcttttgttgttgtttttgttgttcgtgcttttgcaaaacctagtgtcatgaggttttctcattatctttggagaattttatagtttgggtctatacatgtagattattgatctaatttccattaatttgtgtgtatgctacgaggtaggggtccaaattcaatttttgcattgaattgaaaattcatattttcagtttcaaatttcaactgcatattcagttgttgcagcacaatttgttgaagagatcattctttaccacagggaatgatctgggacccttgtcaaaaatcaattgatcatagatgtatgggattatttcagactttagatcttgttccatgaatatgcctatttttatgccagcactgcagtattttcattactgtagctttatcataaattttgaaatcaggaagtatgtatcctccaagtgtgattttcatttgctagagtgttttgactatttggggtctttgcaattccatatgaatttcagaattggcttttcatttaaaaaaatggtagttgagattttcataggaattatattgaatctacagatcactttgggtagtattgccatcttaacaatattgtattccaatccataaacacggatgtattgccattcatatctttttttcttttctttcggcaacatttagtatatgacacttgtaactccttggttaaatttatacctaagcattttatcctttctgatgctgtttaaatggaattattttattaatttctcttttagatggcttgttgcaggtgtatagaaatagaactgattattgtgcttttattgaattatctgaaactttgctgcatttattaactctagtaggttttttttttctttaaagttgtctatatatcttgctctgtgaatagataattttacttcttgatctccaatatggatgcctttctttctttttcttacctaattgctgtagctagaattttcagtataatgtatgatagaagttgttcacaactatccttgtcttctttctgatcctaggggtatagctttcagtctttcaccattaagcacaatgttagctgtggggttttcttagatgccatttaatatattaaggaagtgctctgctatttctaattggttgagtatttttattataaaatggtgttagattttatgactttatgtactgcataattgagattatcatgtggtcttttcattcgattaatgtggtatattttatgattttcatatgttgatccacctttatattcttggggcaaatcccactgtgtacacgggttttttagggtctcctaattttcttcattctttttcttttctctccctgagactgaataacgttaactgacctatcttcaagtttactattttttcttctgctgttcaaatctgcttgaacctatagagtgaatttttcattttacttattgaacttttcagctccaaaatttctctttggctactttgtataatatctatctctctatagatattctctatttggaaagacatttttctcctggttttctttacttatttgtattttttaaagtctttaagcatatttgagacagttgatttaagtatttgtgtactaagtccattgcctaagcttttgcatagagattttatattaatttcttttttttctgtgaacaggtcttttttttttttttttttttttttttttttttagaaaactggacattttgagtagtataaacgtggctattttagaaataattttcctccctccttaggttttgtttccacttgttgcatgttgctgttgtttgctcattagtgacttttctaaagtattttgaaaagtctgtgttcttgatcatgtgggtccattgaattctgtattctgttaatttcatagtcagctagtgttctgaaagttcccttaagtgcatagagccaataaaagaaaaagaaaagaaacacagaaaaagaaaagaggaaaaaaggagggaaagagatttaaaaaaataatgtcggttgggcatggtggctcatgcctgtaatcccagcacttttgggaggccgaggcaggcaaatcacttaaggttgaccatcctggccaacacggagaaaccttgtctctactgaaaatataaaattagccgggcatggtggcacatgcctgtaatcccagctactcgggaggctgagaggtaggagaatcacttgaactcgggaggtggaggttgcagtgaactgagatcacaccactgcactccagcctgggcaacaagaatgaaactccatctcaaaaaaaaataataataataatgcctttgccgatttgctctgtgtttgggccctcattcaatgcctagtcaggccatttacaactctctcttaactttcactacctgcttgtgtactgactgaaggccacctataggtgaaagcttaacatcatctcagatctttttggaccatgaatcctaccctgggtatgcacatgatcttcttaatttcccagtagatgcaagagttttagtgttttaaaagtccttattccctcatctatcttcttttctgacctttttcagtctgcttattgttcatctgaactgacatcctttgccccaagcggctgtggcaaaaacttttacctttaaatgctttcaccacaagccacttgggaagctgccccagacctgggactgctctgaccctgatgaaacaaagacaagaccttgtacagccaggcagccataagacaagtccatacccaaaccacagttctttcagaataaggtctatattggattctctggccctagtaaccagcatgagtctgggcttgccatcttcatggccacttgtcttagtttgctagggctgccataacaaaatatgagagactgagtggcttaaacaagagaaatttattttttcacaattctgaagaccagaagtccattactctgaaatctctctccttggcttgcagatactgccttcttgccgtgtcctcacacagccttttctctgtgtacacatccctggtgttttttaagcctgtccaaattttctgttcttctgaggacaccaatcagattggattagcgaccacccatatgatcattttaccttaagtacctcttcaaaggtatcagagtagtatatttgctacagttgatgaacctgcatacaacatcatcacttgaggtctgcagtttacattagagttcatcgttagtgttatatattctagggtttgttttgttttgagacagaacagagtcttgctgtgttacccaggctggagcgcagtggtacaatctcattgctacctctgcttcccagattcaagcaattctcatacctcagcctcctaagtagctggaactacaggcgcacaccaccatgcccagctaattttcgtatttttagtggagatggggtttcaccatgttggccaggcaggtcttgaactccttgcctcaagtgatctgcccgcctcagcctcccccagtgttgggataacaggcatgagtcaccatgcctggccttattctatgggttttgaaatgtataatgacatgtatccatcgttgtagtattagatagaatagcttcattaccctaaaagtcttctttgcactgggttgagttttaaaagctctttgattattttatgacagttccttattagatatatcttttgcaagtatttttatcagtctgtggttatcttgtcttttacagagcagtaatttttaattttaataaaatccaatttgtcaattacttatctcatatgactctggtgttacatctaaaatgttaccaccatactcaaggtcacctaggttttctctaggaattttatggttttgcattttacatttggtgtatgacccatttgaagttaatttttgtgaggttgtaaggtctgtgcctagattcattttttttttttttggcatgttacttcagtattcataagaaacattgttctgtaatcttctttcttatagtatcttagtcttgctttagtttttgggcaatgctggcctcactgaataaattcaaagtgttccctcctcttcaattatttggaaaagtttgagaaagactattgttaactgttttctaaatttttggtggaatttaccagtgaaccaactggtcctaggcttttctccagtaggtggttttgattatgctttcaatctctttacaagttacacatctatacagacttttataattcagtcttggtaggttgtgcgtatttaggaatctgaccacttcatctaagttatccaattagttggcatgcaattattcgtagttctctgaataatcatttttatttccacaaaattggtaatatcccagtttccattttttatttcattgaatcttcttttttcttagctaatctagctaaatgtttgccaattttgttgatcttttggaagaaccaacttttgatttattaattttctctactctttttctgttctttatattatttatttccacactaatctttactattttcttccttctgttggcctttaattttttttttttaatttttaaggtgtaaatttaggttgagaaattttttaaatgaaagcatttagagctataaattttccttctggtgttcctttcactacctgccataaattttgatatgttgagtttttgtttgtcttggagtattttctaatttgtcttctaatttcttccttgacctattggttatttaaatgtatttaatttttgcatattgtggatttcccagttttccttctgttattgatttctagttttattccattgtgatcacagaagatattttgtataatcgcagtctattaacatttattaagtcttgtggcctaacagaggatctatgttggagaatgttccaagtgcaattgagaatactattctggtgctattaggtgaagtattctctatatgtctgttaagtccaattcatctatagtgttgacgtttcctgttccttactgattttctgacttattattctatccattattaaaagtggagtggtaaagtctctattattgtagaactctctgtttttcaagtctatcaatatctgtttcatatattttggagctctgtttgctgcatatgtgtttacaattgttatatcttcttggcaaattgaccagtttcatcaacataaaatataattcttattgtcttctaacagtttatttttctttttacataaagcctattttatctgatcttagattccctcacactccaccccagcacgcttttggttacatttacataatatatcttttccatcctttcattttcaacctgtttgtgtctttagatctaaagtgaatgtcttacagacagcataagctatgtcattaaaaaaatccattctgcttatctctgccttttgactggggagtttaatccatttgcatttaaagtaatcactgatcattaaatactttcagtattttgttgttttatgtatgtcttataactcttttgctcttcatttccttcaatattgcctttgtgtttagcttatctttttgtgtcacactttgatccccttctcatttcttttttatattttctttgtggttaccaggaggacaatgtatcaacttttaaagttattacaattttatttttttaaatctctcccattcggggattttaggaaggttaaataataatgtaaatgagatacctagaacaatataagcattcaggaattattaactcaattccaatccttcctccacctccacctctttctctgtgagattatagaaaagatgacaaaaaggatgttttctgagccctttaattgttgagaatgatctttgagaaaaagaaaaaaaatgaaagcactaggaatgtacaacagcctggaagtataattaagtgtaaattaaatagataaaagttataagcagaggaaagtatagtagaactcagtatttaaaagagaatcaatgtgaaaattatataaatttatgtaaaataaaactaccagacaaatctgatatccttaggatttttctttctttcatgtgatttctaattgctacatatgacactaaaccattgatctgagctgtaagagaaactggaaattgttctgttatcttttgtaagatttctagaacattttgccctcagacttaaatgccaacgtatttctcacttattgtttactgcttttggatttacatatgatttgattctttcttatctcttatccttacaatgtaattcaaactgatgccaatttaagttcaattgcgtacaaaaactctactcctatgcagctccgccccatctaatttacattattgatgtcgcaaattccatctttgtacatagtttacatattaacatggatttatacatttttatgtatttggtttttaaatcctgtagaaaataaaaagtcaacacaccaatattaaaataatactggtttttatatttgtccatgtgcttacctttatcagtgttctttacatctttatacgggtttgagttactgtcttgtgtcctttagttccaacctaaagaactccctttagcatttttatagggcaggtctagtggtaatgaactctctgagtttttatttagggatgtcttaatttctagctcctgtttgaagtaaatttttctggatatacaattctctgttgattgatttttgttcattttcccttcagctcttttaaatacattatcccactttcttctgccttccaaggtttctattaacaaaattcagcttataatcttattaaagatctcatgtacatgagtggcttctctcttgctgttttcaagattctgtgactttggtttctgatagtttaaatataatgtgtcttattgtgggtctctttggatttatcctagagtttcttggtcttcttacgttggtatatccatgtatttcaagacatttgagtagttttcagccattatttcttcaaacaatctctcctctttggggacttccattttacctatattggttcttttgatggtgtggcaccagtcccctagactttgttcacttttttccagtcttttatttctgcttctcagactcaacagcttcaagtgttctgtattcaagtctgctgactctttcttcttccagctcaaatctgctgttggatccccccttgtaaaatttttaattccattttagtgtttttcaagttaagtatttttattaggttcctttttataatttctttttgttgatattctcattttattacacataatttgtctgatttccattagtttttttctttgttttcctttagctctttgaaaatatttaagacattttaaaagtctttatccaagttcaatttctatggttctgtagagatattttctgccagtttatgttcttcttttccatgggccatgttttcctgtttctttgtatactttctaatttttggttgaaaactgagcatttgaaaatagagccaactttcccagtttctgcagagagtctttatgccacagtattcgttcactgattaactgggtatatctaagcttagggagcagctgagtcaaaagtttaaggtcttctcaggtcttttctgagtatacatgtttcctatgcctgtgtgaaatgttctcaatttccctatataaacagctacttcttcttttttttttttttttgagatggagtctcgctctgtcacccaggctggagtgcagtcatctcagctcactgcatcctccacctcccaggttcaaacaattctcctatacaggtgtgtgccaccacgtctggccaatttttgtatttttagtagagacagggtttcgccgtgttggccaggctggtctcaatctcctgacctcaggaggattacaggcttgagccacagtgtccagcctaaacagctacttttgaatgctttaatttcctgaatagtctcaacccagtttttccttgaggtcttaggtggtccattgtatgtctccacccatagttgcttgccccaggcatctgtgggtctgtggtaccactgcagctttcaccacctgtagctgccacctttccctatctgagatccaggttaggtgagagagatcattccttcacgcagtcccatgacaggttggaacatttcaaataaggtctgttctgctcctctggttgaagggagaaaattgggaaccggtttcccaccttctacaaaccaagatctcatgttgccacgggagtggcagggcaagtgcaagtgaaaatgccatacaattttctaccattttgaacgcgggtttttcttcaatggtcatttgcttggttgctgtaggcctttcactgttttccagagctcccataagattactttagccagttttttgttctttcctgatgcttccctggcagagtaagggttggaacttccaccattttgctgattcataactctgtagtcagttttaaatatattgatacttgagtttgttttatggcccagaatatggtcttggtaaatgtttcacatatactcagaaagaatgtgtattctgatgttgttacatgggctgttctataaatgttacttaaggtggttaataatgttgctcaagtcttctatattcttgctgattttcttttatttatttattttattttttttattatacttttaagttctagggtacatgtgcacaacatgcaggtttgttatatatgtatacatgtgccatgttggtgtgctgcatccattaactcatcatttacattaggtatatctcctaatgctgtccctccctgctccccccaccccatgacaggccccagtgtgtgatgttcccctttcctgtgtccaagcgttctcattgttcaattcccacctatgagtgagaacttgcggtgtttgtttttttgtccttgtgatagtttgctgagaatgatggtttccagcttcatccatgtccctacaaaggacgtgaactcatccttttttatgactgcatagtattccatggtgtatatgtgctacattttcttaatccagtctatcattgatggacatttgggttggttccaagtctttgctattgtgaacagtgctgcaatgaacatacgtgtgcatgtgtctttatagcagcatgatttataatcctttgagtatatactcagtaatgggatgggtgggtcaaatggtatttctagttctagatcttgaggaatcaccacactgtcttccacaatggttgaactagtttacagtcccaccaacagtgtaaaagtgttcctatttcttcacatcctctccagcacctgttgtttcctgactttttaatgatcgccattctaactggtgtgagatgggatctcattgtggttttgatctgcatttttctgatggccagtgatgatgagcattttttcatgtgtctttggctgcataaatgtcttcttttgagaagtgtctgttcatatcctttgcccactttttgatggggttgttttgttcttgtatatttgtttgagttctttgtagattctggatattagccctttgtcagatgaggagattgcaaaaattttctcccattctgtaggttggctgttcactctgatggtagtttcttttgctgtgcagaagctctttagtttaatgagaccccatttgtcaattttggcttttgttgccattgcttttggtgttttagacatgaagtcctcgcccatgcctatgtcctgaatggtattgcctaggttttcttctagggttttttatggttttaggtctaacatttaagtctttaatccatcttgaattaatttttgtataaggtgtaaggaagggatccagtttcagctttttacatatggctagccagttttcccagcaccatttattaaatagggaatcctttccccatttcttgtttttgtcaggtttgtcaaagatcagatggttgtagatgtgtggtattatttctgagggctctgttctgttccattggtttatatctgttttggtaccagtaccatgctgttttggttactgtagcctcgtagtatagtttgaagtcaggtagtatgatgcctccagatttgtccttttggcttaggattgtcttggcaatacaggctcttttttggttccatatgaattttaaagtagttttttccaattctgtgaaggaagtcattggtaacttaatggggatggcattgaatctataaattaccttgggcagtatggccattttcacgatactgattcttcctatccatgagcacggaatgttcttccatttgtttgtgtcctcttctatttcgttgagcagtggtttgtatttctgcttgaagaggtccttcacgtcccttgtaagttggattcctaggtattttgttctctttgacgcaactgtgaatgggagttcactcatgatttggctctctgttagtctgttactggtgtataagaatgcttgtgatttttgcacattgattttgtatcctgagactttgctgaagttgcttatcagcttaaggagattttgggctgagacgatggggttttctaaatatacaatcatgtcatctgcaaacagggacaatttgacttcctcttttcctaattgaataccctttatttctttctcctgcctgattgccctggccagaacttccaacactatgttgaataggagcggtgagagagggcattcctgtcttgtgccagttttcaaagggaatgcttccagtttttgcccattcagtatgacattggctgtgggtttgtcataaatagctcttattattttgagatatgtcccatcaatacctaatttattgagagtttttagcatgaagggctgctgaattttgtcgaaggccttttctgcatctattgagataaacatatggtttttgtctttggttctgtttatatgatggattatgtttattgatttgtgtatgttgaaccagccttgcaacccagggatgaagcccacttgatcatggtggataagctttttgatgtgctgctggattcagtttgccagtattttactgaggatttttgcatcaatgttcatcagggaaattggtctaaaattctctttttttgttgtgtctctgccaggctttggtatcaggatgatgctggcctcataaaatgagttagggaggattctctctttttcctatttactgtacatttattccaccagtgactaaaacaggtgtatcaataaaatctgttccctcaggttttgcttcaggtattttgagggtctgttatcaggtgcataaacaagattgttatgtcctattcttaaattaatctccttataattatgaagttaattttttttttcttgagatgcagttttgctctgtcgcccaggctggagtgcagtggcacaatctcggctcagtgcaacctctgcctcctgggttcaagcatttctttgcctcagcctcccgagtagctggggttacaggtacctgccaccacgcccggctaatttttttgtatttttagtagagatggggtttcaccatcttggccaagctggtcttgaactcctgaactcttgatccacccaccttggcctcccaaggtgctgggattacaggtgtgagccactgcgcctggacctggcccgaagtaaacttctttacccttgctaatgatctttgctctgaagcatgctttgctggtattaatatagtcattccttctttctttgattcatgtttgcagggtatatctgtttccattcttttacttttaacctattgtctttatatttaaagtgcatttcttgtaagtataattggtttcttaaaatccaattatctgccttttaaatgtcatttttatatgatttgcataaatatgattattattacagctaaattgaaatctgtcatcttgctatttggtttctatttatcccatttttttcccctctttttttgctttccttgagattgaacattgtattagttttctagggttgctgtaagaaagtgacataaagtggatggcttaaaacaacagaaatttattgtttcagtttggaggctagtcatctgaaaccaaggtgtcatcagggccgtattctctctgaaacctgtagggaagaattctttcctgcctgttctagcttctagcattttccagcaattcttggcattcctttgcttgtagatgtatccctccaatctctgcctctatcataacatagccatcttctccctttatctgtctattcttctcatcttataagaacattaattactgaattggggcccagcatagattaggcctaatctcatcttgaataggttacatctgccaaagattcttcttccaaataagatcacttttacagcttttacaggtactgagagttagaacctcaatatatcctttggtggggaccgacctcttacccataaaaagtattttatatgattccattttatctcctttttaggttattaactacaatttttttttcttttttttgagatggagttttgctcttgtagcccaggctggagcgcgattttggctcactgaaacctctgcctcccgggttcaagtgattgtcctgcctcagcctcctgagtagctgggattacaggcatgtgccaccgtgcctggccaatttttgtatttttagtagaaacagggtttcaccatgttggctagggtgggtctcaaattcctgaccttaggtgatccacccacctcggcctcccaaagtgctgggattacaggcgagagccaccacgcctggctttataatttttttttaattcagtggatgttttagggtttatagtatacatctttatcacagtctagctccaagtgatatatccctttatgtatagtacatgacccttacagtagtgcatttccatttttcctctctggcatttaggctattgcacacacatacactcaattcatccctttgtgtagatccgtatttccagctgctatcttttgcttctgtctgaaatatatgtatgatttcttttatgactattttgagaaatttggttatgtgcatttgtctatttttcttatgtacttttagctcatcaatcttaagtctgtgagtttatagtttttaaaaacaaatttgaaattatttggctattatttcctcaaatatttttttctgccgcccctgctttccctttccttagggatctctgatttccacctatattactctgattgaagttgttccacgtctcttttgaaaatctctgaaaaatcttttatcatttggataatctgtatttgtacatcttcaagtacattaatattttcttttgcaatgtttaatctgctgttaatcccatgtagtggattcttcatctcaggtattgctgttttaatctctagaaattccattgaagtcttacttgatgaaaaaggcagataaaaacaaatgttggcaaagatatggagaaatcagaatctgcacacactgatgatgggaatgtaaaatggtcaaggcaatttggaaagcagtctggcagtttctcaaaaggctaaacgtagttcccatatgacagcaatcattcatctaggtatatactccagagaaataaaaacatatccacaccagaacttgaacattaattttcatagcaacattattcctaggggtttaaaattttttacattatttttatttaaaatagagacagggtctcactacgttgcataggctggtctggaactcctggaattaagcattcctcctgcctttgtcctgttttctccactggaaaagaataaaactttgtacacttggactaacaactcccgattccctcctttaccaacatgccccacagcctctagtaacttactctctactttcatgaattcaacttttttaagattccacatataagtgagatcatacaatatttgtctttctgtgcctggcttatttctcttagcataatgtcctccagattcatacatgttgtcttaaatgacaggatttaccttcctttaaaggctgactagtacttcattgtgtatatgtatcacattttctttatccacttatctgttgatgggcacttaaattgtttcaatgtcttggctactgtaagtaatgcttcaataaacatgggaatgaagatatcccttcaacatattgatttctgttcttttggataatactcagaagtgagattactggatcatatggttgttctatttttttcagaaacctccatactgtttttcatagcggctgtactaatttacattcccactaacaatgcatgagttcacttttctggacatcctccccaacacttgttatctttcatctttttcataaaagccattatataataggtgtgaagtgacatctcactgtggttttgatttgcattactctaataattagtgtgagcattttttttttttcatgtaccgaatgtcttttgagaaaggtctcttcattcctttgcccattttaaaatcaggtggttttcttgctcttgagttgtttgagttccttatgtattttagatatttacccatttccagatatatcatttatattttttcctattctttgagttccctcttcactgtgttgtttccattgctgtgcaggtcttttattttgatgccaccccatttgtctatttttgccatgcttttgcagtcatatccaaaaaaatcattcccaagaccaatgctgtggagatttccccctatgttttcttcagtaggtgtacagttttaggtcttatatgttaagttttaaatctatttttttatatggtgtaaataagggtctaatttaattcttttgcatgtggatatccagttttcccaacaccatttattgaagaccctgtccttttatacttttcagtatgcagatcttttacctccttaaatttacacctcagtatttaatatttgttgctattatgagattttcataatttccttttcagatagctcattaatagtagatggaaacactactgatttctgtaagatgattttgtattacggaactttactgagtttgtgtatcagttctaccaggttttagttttgttctggtggagacattacagttttttgtatatggttatgtcatcagtaattacagatgatttaacctattcctttcctattaggatgccttttttttctttctcttgtccaactgctctggttaggacttctagtactatgtcaaaaagtgatgagggtcgtacatggcctccatacctaatctgttgagagtttttaccatgaaaccaggttgaattttgtcaaatgctttttctgcatccattgagatgatcatatgatttgatttacaccctccattttgttatgtggtatatcacactttttgatgtgcatatgttgaaccacccttgcatcctaaggataaatcccacttcatcatggtgaatcattctttgtattcgtgaatccagtttgctaatatattgttgaggatttttgcatccatgttcatcagggatattactttgtaagtttctgtccttaaagtgtctttctctggctttaataacagtgtaacactacccttgtaaaatgaatttagaagtattccctctgcttcattgttttggaaaagtttgagaatttttattagttctttaaatgtctggtaaaattcagtagtgaagctgcctaatcctgggctttcctttggtgggatactttttattactggctcaatctcttttcttgttattggcttattcagatttgtttcttcatgattcactctttgtaggttgtatatgtctaggaatttattcatttctttaggtcatccaatttgatggtgcataacttcatagtagtttcttataatcctttgtattttggtgatatcagtagtaaatgtctcctctttcatttctgatcttatttgagtactctttttttctcctagtctaggtaagaatttgttgattttatctttcaaaaaaaaaaaaaaccaactcttagcaactcttagttttgttcatttttttccagtctttatttcaactgtgatctttgttacttacttctttatgctaactttcgggcttagtctgttcttttcctagttcctttaggtgaaaagtgagattgtgatccttcttctttattggcgtaggtttgtatcgctataaatttccattaggactgattttgctgcatcacataagttttgtttccattttcatttgtctcaaggtaattttttatttactttttgacttcttctgtgaactattagttgtttgggagcatattgtttaatttccacatattgctgtattttccaccagaattgattcttgttcttgatttctagtttcacgccattgtaatcagaaaagggatttgatatgatttctgtccacttaaacttaagattagttttgtggactaacatatatcctggagaatgttccatgggcatttgagaacaaaatgtattttgctgcttctggatggaatgtttcatatatgcctgttaagtccgtttggtctaaagtgtaattgaaatccattgtttctttattgattttctgtctaggtgatcaatctgcccatggtgaaaagtagagtattgaggtcccgtattatagtattgcagcctatctccctcttcacatcatttaaaaattgctttatgtatttaggtgggtcaatgttgggtgcatatacttttacaattgttatgtcttcttggtgaattaatccctttatcattatataacaaacttctttctttttatagtattgacttaaagtctattttgtctgatagaagtatagctacccctgctctcaatttccatttatatagaatatctttttccatcccttcactttcagtctatgtgtatgtttagtagaaaagtgaatctcttgccgggcgcagtggctcactcctgtaatcccagcactttgggaggccaaggcggcgggatcaccagaggttgggagttagagaccagcctgaccaacatggagaaaacctgtctctattaaaaatacaaaattagccaggcgtggttgtgggcccttgtaatcccagctactcgagaggctgaggcaggagaattgcttaaacccgggaggtggaggttgcggtgagctgagatcatgccattgcactccagccttgacaatagcaaaactccgtctcaaaaaaagaaaaagaaaaagaaaaaaaagtgagtctcttataggcagtatgtggttgtgacttaaaaaaaaaaaaaaatccattctgtcattctatgtctttttgttggagaatctaatccgtttacattcaaggtaactattggtaagaaactgctagtgtcattttgtaatttgttttctgattgttttgtaggtcccttgtttcttttttccctattgctaccttcctttgtggtttggtggttttctgtggtggtgtgctttgaatcctttcttttatagtatatgcgattaccattgtatttgctgtaaggctaacttaaaacattttatccttaggctactttaagcgataaaaacttgcctttagttgcatacaaaaactctacgctttcacaacccccccgcctttcgtgattttgatgtcaaagtttacacttttttaaatttgtatctcttattgtagctacagttacaattacttcttgtagctacaacttattgtagctacagttgtttttaatagtttcatcttttaaacctcctaataggaataacattgctttacctgccacctttacaatactagagaattctgactatgaattacttataccatttagttttttacttttatgtttctcattaactagcagtcttttatttaagcctaaagaactcccttttagtaattccagtagagcaggcctagtagtgacaaactcccttaggcattgtttatctggaaaagtgtttatttctccctttgccaagtaaagtattcttaattaacagctttgtttccttcagcactttgaatataccatcccactctctactggcctgtaaggcttttgctgataaagccactgaatctgtattggggctaccttgaatgtgatgtttcttatctttgctgctttcagtattctttgtctttgatttttgataacttggttgtgatgtgtcttggtgaactctttaggttgaatctgatcggtgacctcagcttcctgttcctgaattttgtcatcttttctcagatttgggaatttttcagctattacttccttaaatatgctttctaggcctttttctttcttttctccttaaggacctcctattatgcaaaagttagctagcttgatgtgtcctgtaattcttataggcattcttttttgtttttgtttctcattggataatttcaaatgtcttacctttgagctcattgattcttctgcttgatcaagtctgctgttgaagcgttctacttagattttcagttcagttactgtattctttatctttagaatttctattgtttttgtttatatttgttaaacttctcattctgttcagatgttgttttccaaatttcatttttctatccatattttcttgtactttgttgaacttaagaggattagtctaaattatttgtcatttcacaggtctccatttcttctgagtctgttactggagctttcatatccaatgtacaccagaaactttgctaattccacagtaaatgttagaaatgggtttttattgataaaatcagtggcttgctaattagtatgttagaagctggcttgcattggtgaagttgagggtattcgatatacgtatcccggctaaaatacttctaaagaacctctccagctttgcaagaatagagtataggaatctgaggaggcctttaacatggccctcagaaaggacacacagggcttgacctctaaacatataaacatatacagaaacgaggtcagaacatgttttgactgatgccagtagtaacacagcagagcctattgctggtcactgttaccaataccttgtgtaataaacctttcatttaaattgagaggttgtaagccccgaaaaagcaaagggcttacagtacctcttgcctgaacactcctgtgtgtgctttctaagaaatgcttttaaaataatgcctgtggcacaaattgatttgacactggctctctttataagctgctatcaaggttgtggggaatagctaactcttacccactttccctgacaaataggaacccgtagttacctaccaaaagtatgtaaaagctatatccgacccaaaggccagttaggtcaaaacaccttgaaaggagctcagtttaaggaaatcagtggtgtcacagtgccccactagcgtgtaaaagttttcatagtttgactatcggatgtgcttagtttcacagctgagtcttaccttgtgatacttttagagcaaaagtcaaatcaaagatgatttaaaaaaacatttcaaagattcataaagaataaatgcctcctcaggaagtcttctggagttctgcccacttcccttaggtggctctttactgtgcccatctcatagcctgtctccttccacttggtgtattgcagagtaaagctcactgtttacaggggtggtagaaagtgtggtcctttcccagactcctttttcccttcctcttctcatttttcagaagatgtgtttgataataaacgaaacaaaatgactaacacattgagctgagctacataagcagatgtcagtttgacgtgaagagtttaaaagatctatgcattatctggggactcctcccccagacctgaaggatcaggtgctgccttctatgccacctgtgcagacagcaaaagaggaaaaccatacccacgttcagtatgaacaaaggggacatttgaactctgtgtggacccctcattggaagggtgtttcttctcctgctgcatccacaaagagcactccttagccttgccttttgtcagttctctctccaataaggcttgagcagagacaacccagtgcagttcagagagactgaagtctggtgttccaggtctgagtcctagctctagctctctgtgtaactttgggatgtcccaaagtaacttttcacaacttgataggtgttaacttgaattttggatacaggtgactcttagcccatcccctctctgtgcttcagatatgtcatcacttgggccatatgacctctggacacctttcctactttccacaatttcagagcagcagagcagactggagctcctgctgcctctgagcttcagtgaattatcactcgttggagggaagcttcaagcattttgttatctttcaagagcaaacacagtgtctgtcagcaagaatatgtagcagatgctagtgaacagcagtgattagggttgaatgctggatttaaatatggagcttaggctgtgaaggaagcctgaagaacctagagccccatgaagctgccctctgtgagatctgacaccatttcccactgatgagccatgggtctaatgggtagcactgattccacatacagtatgtgagtgcaatacagtgaaagcaaagagaataaaatgatggctaacatgatgttccaaactttaaacaggagaaaaacacacaattccattatgtataagaacccacacagagatcaggagaataacctcattggagaatgaatgacctgtgtggggaatttagggtagagttgagattgaaaaaatgggccgaagtcaggtggccaagggccttaataccttgtatacaagatgtgtagtcaaggaagaccatgccttacttatgcatcaattcccttgggcctacaaagagctgcctagcctgggactgttgtagagaaaagctacagtgttccaatgacacagggactcctccatgtatgtatgagtgcccagctggctctgtaataaatcttatttttatttattaacttttcttgcgcattggcttgatgcatcagttggaagtcagaggccaaacgaagtgaacactgagccaaaggaagttctcagcctttagggaggcagaattcactttaaacacaataaacaaatgaactcacattatacaggagagagtcagaagatcccagtggctggtgtcatcgggccatatttgcccgaagtgcctattccttataggaacccactcccagggttgatgggctacatccttaggaggctttatgcctatgttctcctgaccactggctcctccagggctggccttttttagtctctctgtagaggttcctgtagctggttggatataggctttcacagaagggtcagtgccttgggtctggttacaggactgttaatcttgctttgttaagagtaatgttatttccccatttccaaattctccagggagatggaatgtcaaagatagtatgactgtagcacctaaatcctgggttccagaagcagagaagagaatacactgaagctgtagaaaggcctattagtccatgtcagagattaactggatgcgaggaccatttctgggatggtgtatacagaactggagaactggatagggagtcagaaacaaagagctgaagatgatgcctttgaagtttccaacgtggataactaggtcaacagaatattactcaagaaagtattaacataggattgagatgcagtcagtagtaatggaattgaaatttcaaagtatatacctcatggatctcagggggtgttgagctgatcacctgggctaacactcctatgaccctggggaaaatcaaatgacctggtactgtagccatggtaggggtgtcatcaccttaatccaactgggacagtgctgtttgatattcatctggaacttggtgcagacccacattttgctgggtttcaccacaaccaaggcttttttgattcttttctcttttaacatcagtacatcactgcaaagttaatcctcatataataggagatgaaactaattgcttataaaaacaagatttttacaacactaaaattgttcaagcatatgggcatatttatagttgcaggcagtgtttcagatgcagactgttcttggctgcagtggttgtttacaggcagcatctgttctgattaaatatttgatgattatccccgaatgttttaaagcatagtactgggctctgctgactgtacaacaaactggcatttttgacctatagggcactgggctaggagatacagttctgagggaagtgaaagatagttaacaactgcacaactgaccctttattagttgcaataaagcaatccaaccacccaacccactgtgatggctttcctactatttaaggttggtggtgtcaaagagacaccctcctgtacagtgtgcagtgaatcaacatcatttccacaaaacctccttcctgcacaaaggaattatcatactttgttacgaagtaaaattttcctgtatcagtcacaggagttcaccagttaagatactgttagttgaagacttctggggtgacttaatgaaatagctcagccatctggtttaaaaactggattcttctatccctccacacagctgtccatgcacctgcatcatctcaaggctggtctccctggtggtagaaaggctgacagtaaaaactggagccacatgattccttgcttaggcagtgtttcttcatactctcacatgagagcaggcatgttctttccctaggctcacagtaaacactctgtcaaatctcacaggcccaaagtgcttttggttatccccattccaagccaatctgtggcatggaagatagcattaccctgactgccttagactaatatacctactccacttctggggctgggaataattttgaggtcaaccatccaaactgcatgacagccattccatggaagaggtatggcctaaatctttggggcaacctgaattcatgaaaactctcttagatttatgtaactatttttagaattcacttctgtatcatttaattttactaataaaaataccacctttaccctaaatgtcagccaagcgtaaggctccgttgggacagaaggaactatcaaagctttgtgtttttatacattagcagcatttgacaaagaaataactctgaaggaaggagaataaccaggcagagtctagatgcatggaaaagaagtctttgagaaggcttcgcaggctgaaggaaagggcaggactacttcaggaatacaacgtttaagtaaaagaggttggggtctgttgatcttgaggagagatgaggatggactggaaaataggagtgagatatagtaggagaggaaaggatatggatgatgtatatactgtatgggtaaccatcagtcgaccctaagaagataatagttgttaaatggttagctatttaatgaaagaaacctgaacaagtaataatcttgagttgcaaagtggctggagtcacacaacagactaaatttctggtagaacaaatccagcagcttatgtgaaggttaccatgtctgaagctggatagaaaaggtctaacttccaaccaaagtcacagttcttgagctcggtacacagagacagactgctaacagctgatgtgtcctcagcgagagtgtcctcatttatatcctcgttctcttctgcctcctttttttgttttaaacttttgggaagtctcatcattcaatacagttctaatatatcacaataacagaggcggcccaatttctacaaattgactaattctatccctgaaaagttcatacaataaaactatacaaagcatcattttcaaccatcctatagaaaaatttccctattaaattttaactctaaatcctctatctctgttaataaccttactaaaaacttccccatcactgcctgccagggagatcaaaagaaaccaaatttaagaaacctccaacacctgtacctgactgaaaagcaaacatacagacctttcagtcctgcccctactattcagctcctatcacagagacatggtggatgtccccttgggaagcggatagctcttaggtggaagctaggcctgcaataagcaggccaggaaacatgtctcccaggccccatactcttggcagaagcacttggccaccagacagcatgtcctgtcaacctacagagttcttaaaaacaaacactgggacccagaatagtaccctgtggtcatagtgcccacagttcactaagcaccctcacaggtctttgacagaacactgactgccaggtcacctggtgggcagagaaatggaagattcccaggcccaactagcatctcagggaagaaccacaagcagagcaactttcagagctggtcggccagcgttggcacccagggaagcaagtgctattccatatttggagagaacataaaactcaaggaaacagagaagtcctactcaataccgttctcaactgaaaacaagagaacttgcaaaaaagaaacccagttttctggagtccatggagaaatatgaagccaatgctcggctaacagagcagaaagccttttataaataatgccagtcaaagctccaaaggtcagagctgatgcatgccggattgttctgacaatttcttaggtttctaggcaacaaggagctggtcaaacagctcacctccaaggacatactttataataccaccctggtagacgagagcgaggcagcagtgaaggcaatagtcaagaacaggagagggaggtaaagaacaagatgctcttcagacagtctggacaaagacagtccctacccagctctcaggagttgcctccttaagaaggtggaggcccaggcagctcccagggtgaccctaaagacagactccaaggaaaagggtgtgagggcaatggtgaatataaggaggtcccctttcagtaggcagagcaagtcagttcaggtcttatttttagagtctctgaacagtgaagagaagctttctgtggacagcattccaccaccatgggaggggaaaggtgccatgagagacttctccagtggggcatacaagcattgtgcagtgatccccaagatccagccactggcaggaaatcgaaaggcaagcttcttaaatacatagtatatggagacagaagtgtggagcactcccaaaatgaaaggccaagacccaggaacaacctccacaacctggagtatatacaaatgaacccagccctgctgactcagatccacaccatcctaaagcaggggtttctcatgaatgaaagggctaagtatttggtggagagaatgagaagtctcccactgcccacacattttttcttcagagatttcttttccaagagccccttgaataaaggaagggagggagcactgaatgccccagaaatcagaatgcatggtgcgggaagacgacaggaaatagttctcaaagagatcagaaaataaattggaagcaatttcattaccacaaagaacagacattttttctaaggcacccctcccctttccacccaattgtcattcagcaactactgaatacttaccaatgaaaaacgttacccctgacctcaaggctgctcctgagctctggtagaaaatgctttccttgtctataaaacatggcaagcaaggcaggatttaacagtaagggcacagtagcactgcaagccttcaaatggaaacctggagacaagcagtgagaacaggaagcaaaagcagggcaggtgaagctaggaccagggcatctggaactttccacacaggttggatctccatgccagacaacagttttcaaggaaaaatatctaagaggaacatgactttgggaaactttttggcagtactgcttactgtatactagagagtaaaagaatttggggaacattcaccaatttgcttcttcaggggcttgggtagggaacgtgaacaggaacctggctctaatttctgaacttttttatcagtaaaaacaatccaacaaacgaaagctagtcagtgagagaactgggagggtctgccctccttccctgagtcaagccttctggggggacctcctgacatttaattaagcaaagacaacgcccactgaaggaagctgacctgaaagtgacacgctactgtgaaatgagcatgaagtgggagcttgttacatatatgaaatggccagcgatcctgagcaaagcgcttcagagcttgagacctaagtcttctcatctatatactgagggctggacaagatgatctgtcaagccatttttatccctaatccaccaaaatccaatgctttagtttattgtcacaaaagcaggtatcgaatggctatcctgcagtgcctccaatcaacattcagactttttccctgaggcaatataagataacagttaacatgtttttatcaattaggtggtcatgagataaatatatatgggaagtggtagtttttcacttaaatgcatataataatggtacagctctctttgaatagtatttgtttatttcttaaatatttaagttcctaaagacggtaagaataacccaaggaagtgaaatcaatgtcacaaagcacatggctaaataactgcaggtttgcagtgccatgtgtgagatcagatgacagaagggagaactacctttaggcagaggcttctcatgtcccctggagtggccatgtgctgttctacatgactacttccacttcggttatgtagaagctatttaaagcacacagatgtttgtgatgagaaaaaagccacccttaattgaataatggaaattataagcatgatttgagggtgggggtggaggtgggagtagagatgggtagaaaggagtgcaatggaaacaaaggagccttcataaaattcaagtcacttcttaggataacgtgattgatttactcaccaccttcttaggaacataaagcaaacaagtgggttttccttttactgcttttctgaaatgagctacactcaagaaagcagcacgggggttgtgctgtccctgcacagtggcaggagagtatgaggagcaggtgaatgccacaacagctccatccaagatcatttttcacatgcaggaaccattcttatactaccctttactggtaatttctgtagaaatctggaagtctggttgacaccctcctgtacagtgtgcagtgaatcaacatcatttcccttggactttgcaatcacggtggcattcatacattcattcaacaagtatgtatgtacaggacagtggagtaaagaaaacagatagttcttactctcacgaggcttaaaatttcaggagggaactaggcagtcatgaagtaaacataaaaacacagattgtaataggcactagagaataatgaagactttggggaacacaatttaaattggaaaaatatctcagttccgttaacacatgttgaaggggaatagcattgttttgctggatttggggcctggatgcaagcatgttgggtatgcattgtagggtaatgcatttccttccatttgggcccaagtgtatatttaccacccagttgtgatgagctgggatcctcctgctcaatctcagcttgaagcacttggaggttatctgcctgctgtgggtgattattttggagcaaggtacttcatttgcctcaagaaacagatttgataccactactgtgcccttttggaacagagaagtaggcaagaccccagtgtgaggcagagtgatgggatctttagggacataattgatgatgtaactgatgatgattttggagtttatacatttccaaagtttcaaaatattttcacttggttgatttatctttatggtgatgacactacgtagatatatgcccttcttaaaagttacagtaagaggctgggagcggtggctcacgcctataatcccagcactttggaaggccgaggtgggcagatcatgaggtcaggagattgagaccattctggctaacacggtgaaactccgtctctactaaaaatacaaaaaaattagccgggcgtggtggcgggcgcctgtagtcccagctactcaggaagctgaggcaggagaatggcgtgaacccgggaggcggagcttgcagtgagctgagattccgccactgcactccagccagggcaatgagcgagactccgtctcaaaaaaaaaaaaaaaaaaaaaaaaaaagttacagtgagagttgacattgagaaaagggaggcccagccagggttatgcaaagacacagtagggaggaggatgggaagtttctgagacaccccagtaactgcatgggtccacaaagcacatgtacagtctgctctatgcactgcaggtacagccacgaataagacaaagtctctgccctcatggagcttggtgtctgtctactggagcagacaaaaacaaacctgctttttctcatttgcctctactgtgggttccgtgtataactctcaaatgctagcatttccatgcattccatccttgtccttctctcctctctgccttttccaggagaattttcctagccacaggttcagctatggtctatgtgctggagtcatacatttttatcttctgtgtacgtttttctctagacccatatttttaattgccttaggacagctctccctggatatccctcagacacaactaaaacatcattcaattgtactattaataattttcccttcaaaatccactcctcttctctatagacctaacagcaacactgtccgtccagctctcaaaacctgaaatgtgggagttgtctttgactcttatctttcccatgcatgagcactgaatcaatcagagtccctagcatgtcttggactgtggcctcccttctatcttcagattatctgcctttttagaattatggcaataacttaactgcaaagaccacatgtgcacttgtgctctttcagtttgtaataaatataaaatgaaaagtgattatgtctttgcttaacgtctgcaaaataaaagtccaaagtccttggtatggtctataaggccctcattatctgacctgcctgccttcccaatctcatcttaatcccttacagcctgacactcagacatactagactttccacctaactcactcacatacacctatcctgtatgtcaagattcggcttgaccttcatcacctacctatctgcagtatttttggatctgaattctctgcccacattgtactttcacatacttcttttacccttagtggtttgtacttatgcctggtctaactagattgtctccctgtaacagacttcttgattcaacaaagcagctctgaatcagcctgaaacctatggcacactgcaaaatggcaaacattcaataggtatttgcccagtaaatgttaatgaaaggaaaaaaattcaaacttcagttggaataggattagagacaagttaaaaaaaagtttcccatagaaatcttcctcatcgtaaatatcatcatcctaaatgtcccgagtctttccatgggtctaatttaccaaatcatgaagactctctttctcactgtgtatgtgtagtgggagaggcagagacagaacggtttttttttttttttgcaagtctgctctctggattcgtttttctggggatgaaatctcaggctatgtagcttttctggtccctttttggtaatcaacaaatcatcagtttctctaggtattaaaaagcctacacttttgaaacaccaagaggccaaactctctcttaattgaaaacataattctgcctcttactgaggtctttgggtgggaagcttaaagacagcagtgttggggcaattggtttttcttttcctccctccaccttctttcccattcaagagctgttgctgccctttctggaggggagggaattagaaaaaaagatcctgccttacccagtcactgttaattattactttgagccagggtggggaatgactttctttcctggaatacaccctgctggaaaccacagctgagattctctaagctggcagcttccaacctctcctccctcaacagcttcaacattatatggcactcagctgcagggtgcctggctccaggccggcagtccatttgtgcagggtagttttcaagatggctcatagcccatctctttcagtgaccagcgtggcctatgggaaacatacatcttgactccattattggtaggagtactctttagttaactgccacaggccagttcagacacgtcttaccctgagagccttttcagagtcaggtaccatccctgtgtcccccagcatctgaagatcacaggtgaaggtctccaagtagttcacttaaaaaatacctcagtaggtttaagaacagggaaagctcccaattcattctgtcaggagtatgactctcatccccaaactggacagatatataatatgaaaactacagaccaatattccttatgaatatagatgcaaacattctaaacaaaatactagcaaaccaaatccagcagcatagaacaaggtttatgtagcatggacaactgagattatcccaggaatgcaaagttaattcaatatacaaaagtccattaatacaacatattaataaaggacaaaacaacatgaaaatcttgatacagaagaagcatttaacaaaacccacagcccctcctttttttttttttgattaaaaaacactcagtaaactaggtttaataaaagaatttcctcaacttgaagcaaacccacagctagctaacatcatactcaatggtgaaagactgaatgctttccccttaaaatcaggaacaagaaaaagatgtctgctcttgtcacttctatccaatattgtactggaggtgctagccagcacactaaggcaagaaaatgaaataataggaatctagactggaaaggaagaagtaaaagtatttctgttcacagatgcatgatctcatatgcaaaaaatactaagtgacaaaaaaagaattttagaaaagctacaatatacaaaatcaatatacaaaaattaattttatttctaacaccagtgatgaatacaaaaataaaaattagaaaataatcatataattaaagtggcatcagtaaaatacttagaaaaaaattaaagacgtcaagacttgcacactgaaatctctaaaacatcactgaaataaagacctaagcaaatgcaaagacatcccacagtcatggaacagaaaacctaacaccattaaaatagcagttatttctcaaatttatctaccaattcaactaaatccctatcaagatcccagctgttttgcaggaattgacacaatgatcatataaaaattcatatgcaatgcaagggacccaaaacagacaaaatgattttggggaaaaaaaaaaaaaaaatggaggacttgcacatcccaaatctaaacttactaccaagctacagccatcaagacagtgcggtgctggtataacgacagacatatagggcaatggagtaagactgagaatccagaaagtcttatatttatggtcaactgttctttgacaagggtaccaggaccattcatggggaaataatagtcttttcaacaactggtgcttgggcagatggatatacagataccaatgcacttatgcaaaaaatggatgaaataggaaacttcactacattctactgcatgctcggtcattttcaatcatttaggtggcaacactgacaagataacagaaagatggaggtaataacatgtgaaaggcaaaatggtttgtttttttttttaaaaatgacagtctctatcatgaatttacttacactccaggcaaaggttattagaagaaaaaaagatgtaagaaaattccttaactgaaatgtggaaagagtatcaagaggagaccctaagacactctgtaagaatcccagtgactcctcactgttcaactaagaaatgtaccccattatgctgtgctaccacggaagcattggaggcactttgggggttgatgaagtcttcatggatgaggtactttaaatatctggtcatgaagagtatttgagaaatatgacaagtgaagatgctgggtaggaatgcagcgaggaaagtgtgtaacacaaggccaaattggaaaggtcagtgagggggcaatctgtggaggcactgaatgcagaggatattaaaattagcaagatagtgttctagcacaatgaaaaggattggaagagggagatgagagtcagggagtgaaattaggcagctgctaagaatgtccgggtgagtcagaggatcaggacctgtaagggcagtataaagaaggaaggaatgatgggagaggtttagggggaaagaagtgacaacctgtgacaattgaggaatgagagattttgatgattggggtgaaggttacatttcttaaaaagaaacaaagaatggtcggtcgatagagatgcaagatgaagaattttgtttttaggactactgactatgaggtaacaaagaaatcccagagacagaggtctgagcaaaagatatgggattggggaagaatctttgggagtgactgaggttgactagagggaactgggcaagaacaggtaaggactttaagcagaattggaggagtatccatctaaaatctgggtaaactgggatggaaaaacaagtagccagagaagcaacagcccaagctagggtgttgtcaaggttatagatgttactgattttggcaatgaggagattataaggatccttgaagactgtactttcggtgaaactaccatgcattagtagcctttcacagtgatatctaccccacacctgcatcaaaatcttgcagtgcctattaataaatgccaacccactaacggggaggggagaaaagacttgggactctgtacttgtaaaaccctctaccctccccaggcaattctttacacactaacactgttgaggtaaaaggagacaagtgagggagcaaatggaaggtgtgtttttggattatacagtggctcatggaagggtgggaggggtacagatggcccttagacactggcggaaagtcagagaaaaaaaattacagtaagcaggtaaagggatcaagactacacagggactagtcttgggacgacatttcttcctctgacagtttgtatggaattcttgagaaaaattcctcgaaggggcctgaaatctcagaatggtcatgtttttaatgggaatagggagtgatgctgccccattacaaccatctgttctaacagaatgtctgtaccgaggagggatgagtaacatcggcaagttctgttcgaagcctttttcaagtttctttttgatatgtatctatctatctatcatctccctatacaagcaagcatccccaaaagtagttgtctcaggaaaccagggttaggatgaccagctcatttgctggagctgccaaggtccagaaaagtttggctgcaggctgttttcatgttttatgtatgtttgaaatgttctataataataaaaggttaaaaaagtttacatttatttggaaaaccagttactttagtttatggttcctttttttccctccagagcttcctggagattgaggtctaattcaaagaaaaccaaaatatataatagagtacctgggcaaaaaaagtacttttataacataacatttggggtagaggaagtatccactgtagtcaaaatgtctatgttttgctcttccttattgttcagggacattccattaaatagtaatgaaaaggcagcaaaagtaagaggagtgacaacatgcccggcataattaagcaagctagagcagctattctgtgcaaccgacgattttttttctctaaaattttaagggtaggttcattctgactctgttaaaagtctacttgatgtgaacaactctatatctgataacctatttcaattaccactttaaaacttgtcatatggatacgttattacaattgtagaactttaataaataccataataataaaacttgagaactgaagagcacacatttcttcacgaatttattatataaaacgccctcagagtatttaatttctcctcactttaattacacattaagaagcacagtggatgagaagcctttaagatgactacagttgcacgaaggtccctttcatcaaggtagcgtatgtaccctaacagtgttctaaaggctggcccagaaaaaccccatgttaccttatcacaatatggaaagcattgtcttctttttccactaaattaaattatggtgaaaagtgccacagttttatttagcattatggtacataacaaacagttctgtctcaattatgaaaaaaattaattaaaataatcctgaaagacatcctttttctccccccaatgatttgaaagctgcatttttcctgccaatttcaaacaaacaaatcatcaggttgatctacagtaatcagttaaaacaatcagtcaatcaatcaatcaatcaccaaggcacaagctcagcacattagctatagcttgtagcaaaaggatatatcaatgtctcaccttagttaaaaatacataatccttttattttataatgcaataaaagaaattaacaacatcacatacacagaagactaggaaaggggaaactacttacttctggaaatcagtaatgtaaacctacttgtacttttccatagtacatgaaagtaacgtttaacatgttttgaattaattaattaaatttaatctgtggggctatacaatgtaattcttaggagtaatagtttcattcatttccaggtcagcttactgtatgattaagtaacacaaggcacagtagccatctttttcattatgttgcaacactgatcacgtgcctcgataaaatggctgattcaacaagatgatggcaacacgaaggggagactttggattgtctatttaaaatctaggtaataagtaagtaattaataaaaactctatcttaagtgcactttcacatgctttttgtttataataaacaaacaacaaacttcctaactttgttgcaataggcttgactaccatttcatttggccaaatgcactttccccagtaaacttaaaacaacaacgagaacaacaagaacaaaaatccctgtcctttcatatactaagaaagaggattggctactgaaacagttcattgcaagacacatgaagacgacatactgtggcatgagttgtttttgtttttaatttgttgtgctgttactaaagttctgagggctgcagttaaaacattccaatttctcccttccttccatctttctttattgattgattctcaagattttgcacagaaaactctttgggggctagaacagcagtaattgcatcacactgttttcaagacttcaagtttcaaaagcaaatcattaaaaaaaatacagttcctgatttgagttagatacagggacaaaaaagtagcacatacttgaaggttacgtggtctacaaatggtggcaatattttccttgggagagtagttctgttggtatatattttttaaatactcaaaaggctcaacctcaagcagtaataaacacaagcaaaagtgatttaacccttaaaataaatattcagaaaaacctctctgtacatacaagtgaaagaatatgtaacactttcacgcaaaaaaataattataataataataaaggatttgttcatatatgtagctgaaatctgctgttccagcccacatgtccccaataaagaagggaggcacagacataggtgactactgtggttgactatcttacagcctttttgtactgggacactatcaccaccaaaaatttatccctcgttatatttttaaaattttttaaattttttctttttttttccttccttttttttgttttattttgttttgttttgttttacagcatgccaaatcctttggcatacgtgatggccttcaacaatctctctttaagtttttctttgcttgagtattccggaagtaaaagcacattaaagcaagtatgagatgtaggtaacctaaatagagaaaaggggaaaaaaacaggaaaactgtaagtcatgggaaatacacttagaattaaatgctcctatttttagattgtatatagttgagacggtctgcaatgcaaactatacattaatgcaaatcataaactttttgttgtgtaactaccaagttgcctttatcctataaattactcaaagctagtgacgatgataagatactgtatccattgagtttttactacataacagataccattttaggtactgaattcttacagttcatttaactaaatctttccacaacaaaaccacagagaggacatcagtaaatgcactttagaggttaggtcactgagaagtcaagtaacttcctctaaggtggagaaaatactcaaacctgttttacaagactgcaaagtgtgtgctcttaaatgcttattagaaacactgctggcaatatgactaagaaaatgatttgataacaggattctagcacaatcaaatgataatcttccgagcctcaatgtaaccattctaaatagatgatcatgttatatggctttcaattaacaagctgggaatcaaaaaagtaaatgaatcacactaatttgattccaaaccaatgtgagccccataataatttttaactagggcaatttcttaaaagtttcctcacacaatgacagcaaagtattttctcaattgtctaatatgatttggggatttgtatataaaatcacgaatgtgctcagaaactataaagacagttcatatgtatgtgacgaggaatgcaaggttttcggtaggtatacagtcacaagttaataattacctacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatgagaaaaaatacaatactggtttcagtttggcattcattatgactggtactaacataagcttatgatttgcattaaaactatattaagagacaacttggaagttaattatcaggatagtatcacttctggtgatttaaaaatttccaagcaaatttatcctgaacagctctgaatacgtaaaaatttagattagattacaatatagtaaaatattagtactacaatagtaaaaaattgagaaaacccaagtgtgtagtaacaggaagtgactatttaaactatggtataatcacattatgcagtcagtaatgagcaaaagacaaaatcctatgaattgaaaaagactgaaatgaatatttggaaaaattaactccaggtgcctttgggtatatattttatttatcctttctcctttattctcctggtctactcatcctttaactcaactttgggaggaaaaagtgatacagattaaggacaaaagaaaaaaagaccccctgccccaaccaactggccccaaatgcaaacaactacatacctaatagcagtaatacaaaagttcaatttttatatgaactagaatactttaaacaagtaatatgtgctgtgtatggaggaggagaattattctgacatttctgccacctgcagttattttaagagaatgatttatcctgtggcattcctaaaatctatgtaataaaagctatgttttaatgacacttatgttagtttgagttctaaaaaacgaaatacaagctcataaagtgcaatcttgaagtttatttgaataatcaggcatctataaaacatatatacactagttcatagctaaataaatttttttttttttgagacagagtcttgctctgtcgctcaggctggagtgcagtggcgcgatctcggctcattgcaagctccgcctcctgggttcgcgccattgtcctgcctcagcctcccaagtggctgggactataggtgcctgccaccacgcctggctaattttctgtacgtttaagtagaggcaggatttcaccatgttagccaggatgggtctcgatctcctgacctcgtgatccgcccaccttggcctcccaaagtgctgggattgcaggcatgagccaccgcgcctggcctatagctaaataatgttaagattaaaaaattaaaaaaaaaattaaaagtatttttagttgcttatataatataaaatgcattttaatagttatttagaaagttctttccagagccaggtgtggtggctcatgcctgtaatcccagcactttgggaggctgaggccagtggatcacctgaggtcaggggttcaagatcagcctgaccaacgtggtgaaaccctgtctctactaaaatacaaaaattagccgggcgtggtggcagatgcctgtaatctcagctacttgggaggccaaggcagaagaattgcttgaacatgggaggcggaggctgcaatgagctgagatcacaccattgcactctagcctgggaaacattttgagccatctcaaaaaaaaaaaaaaatttcatctcaaaaaaaaaaaaaaaaaaaattccagaatgttagcagagaacatcacacatcccaaaccagtaattacaattttctaccttttgatattctaatatcccaacatactatttttattaccaaaatgctggcatttttggtgctgcaacaccatttatctgaaataacttaaaagttttattagaattcttggctttctccaatctcttgccacttcccttccctgcccctttacaaatccaccagtcagcaagagaaaacaaaaaagaaacacacaacaacagaaaactggacatgaaacttgcaggagcatctctcaggtaaggaggagaggaatcggaccccagttcatgatactccttttggggaggctgacaagagaaaacaagtctgctgtagcacagaaccctccaacatagcagaaaagctgccctgcagtgggtggtaataaagcctcctcacactgcaggagggctctgaagcttaactgaaccagctacacctaaggaggtagaccaaagagcaccaccgaagaacagagacagaccctgcaggaagtggcagatcagcagtggtcacaatgaccagtctggctatattattacggagctgtttctttggacttttaaacgaaacctttaaaaattttatacaatgaaaacagggaacaaaatgcatctctattcctataagtgttatgtgtgttacattaacattttgaattaaacaagaatgcatatatttagaaagctgaagaaaacacgaagaagcttccaggtttccacataaaagtggtggctgtatcgatcacatcctactcacatctctaaaaatctacccggatcaaaaaggggaaaataaaaaaaaaacctatgacttaggtctagagtaaaactaggagacagaacaataaactccaaataccaaagaagtggggaaatgagcagggtccagcaggagctacatcgaagagtggcttgtgcggaatcatactgattagggatcacccaaggccagaccccaccccctccaccacctgcccctcaacagagcactactgagtgtaggttagagcactggcaacagggtggttaaaggaaggactacagaaaagtacttgggtccagcaatggcaggctcaggaaggcaccatgcatgaaaggaaggggggtaccttcaaaagcagaagtgtcccttaagcggctgtaaaggggtggaagcagctaatgaaaaagagtcttcattaagctacatggagctgaaaaatcagaaagtggtgattctgcccttactaaagcaacagaagagggatcccttcaaccaagacccctaaagccacacaattcctccctgctccaccatgaggtctagtaataacaggtccagaaaaaagtaacatctgttatagaaacataaacaagaaaaacagaattcggaagtctaaataaagttattatgggaagagtctggcgaatgagaagcaaaactttccggtagacaaaagtagaccagaaaaattcagtcataaacaatgggaaactagtaacaccacattccaacacaaaaggagaatgcagcagcagacaagacagaccaccagtgatgagaaatacaaattcagaaagaaatgagcacatcttaaataaaaataagaatactaaaaaatactgaagaaatatgcaaggtaactttggaaataaaggctaattttattataaggtgaccaaagaagaagcagtatgactaaaatttctactgaggacagactctagaaaattaaaaaacaaagaaaatgaataaaataaacataactaaagaaatgcatatcaaagcataataacgaaaaagattaatagctaactacataaacataaaacctttcatttggcaaaaaaatcctataagcaaggttaaaagacaaatgacaaactgggaaaaaatatttgcgaatttaccagcgttaaaggactaatctccttaacatataaagtttctaaaagtgaagaaaacgaccagctgagcagcaaaatgagcaaaagactatataaaactataaacagctctacaagaaaaaatgtccattatcattcataattgaggtaagctcaaaaattgtaactatagtaaaataccatttctcaattggcaaataagtaaacatccacatttaacaacacattctgtttggaaagttttaagaaaagagatactgttgtaaactgctggtgggaatgcaaaatgttatttttctgtgaaggaggatttggcagtatctagcaaaattacacatgcatctaccatttgatgcaacaatcccacttctaacaatttatcttaatgatactttacatatgtacggaataatgcatgaacattaagaggattcactgtgacattagtactaacagcaaaaggttaaaaataacctcgatgcccataaatattgataagctatgctgcatccacacaaaggagcagtattcagtcatacaaaacaaaatggaaaacacctctgcaataatgtgggatgattctcaggatacactaggaagattattttcctagttctgtcctctgataaggcctacaagcagtaacgttcaaaagcaaaggccatactttgcatctaaaatctgacttctaaatgccattctccaacaatttattggaaaaataacttattccaggcctgagaatgaatgttcgagattagttagaaatctcaaaatctaatagggtcattttaaaagcacacgatagctaagcaatttgaatatcatttagagtaatgactgtgaaacgtattaaatacaaaaacattcattagttcgtaatattcaaaaaggcagcaaaaaccaaccaaaaaacaaaattaaattgtcactattaaaattattacattaactccttactctaaaaaccttaaatctattttatcatgccttttctgtagaactgtttttcaaggtaatcaaatggcacccaatgatgagaaaaaagaatgccaggtatatatgtaggacaagcagatggaaattttttttccccaaacagccattttgcaatccccaatgagataacagattaaggtaatgatactgataaatacgaaaatcaggtgaagggcaggtagcaggttctggaaagatgatgatggcaacgacatagttattattattattattttttgttttattccctcccaacctcccctacaaaaacagcaaactgaatgagaaaaccaaagacccagagacattatctacaacaaaaccatgaaccattgcatataattggacagaaacaaagctctgacaactacaagactggttagtaaggaagcagatgcaaggtaactgactgggtttctgacagccctgagaacactgccaacccactggaaagcacaggccaatctgaaaacagggcttaaagttttaaaaagttctacaggatctaatttgcagatgattacaaagggatcatgatgtagtaagctctgggcccttaaaacacccgaataccaaacccccaccagaaacaaaccttgtaccgaggaaaaacttctgggaataacttccgaatggaccaggtcagtaataaaaaccaaaaaaaagttcaaatatatgtgtgggatagaggagtaaagaaggcagtttcagaaagcacaagggcatatttttgaccattttacaaaaacacagacttctcctcgtccctaaaaagcgaaaaaagttatcctggcccatctctccctcgtccaagtatgagaaactcatttcactcacacacacacacacacacacacaaataaataaataacagaacagagtgaaacagtgtaattattagagaaaaagtatgtgcatatacatgagaatggtgtccctacagaaaatcaaagtacatgaaacaatatgcaaataaaacatgaaaactgtaaacaaatttcaaactgagctaaaaagaatttaaaaaataacaaatcattagaaatgggaaatttctgaatgagggtaactgaaaaaaaggaagacatgacacaactaaggagtaattagaaatgcaaggaaaaaaatcccatcaaatacaaagaaactaaaactaaactagaagaaacataaaggtgaataaaacagaacaataataccagaacctgaaggcagaagaaaaatcttaaaatcaaaaagaaacaaacccatgcttgagactctccttgctggtccagagccacagtgctgctgtgtactggcaggaggaattctgctataattggtcctggcagtgttcacctttcctgcaagtgttccacagcccagggacacaatgtggtcaggagcactgccaggaacaccagcaagggggagcctgccacaacaggcacaaggctcaggaagcactctccagcccacgaaaatctagtgggggtcctcttccctcacccaaacacactctgcgcagct

SEQ ID NO: 2 - DNA, 110638 bp, Homo sapiens
  misc_feature 1..110638: note = Human SNORD115 genomic sequence
  source 1..110638: mol_type = genomic DNA
gggtcatgaggaggtttgatgacataaaaattatgctcataagtattatgctgagacccattcttgggaggagatgtgggggtgtcctaggctgtgcaaaacatagctatggaaacagaagttattaagagaaaagaacaagcagtcagtgagaatttagataagagactgtaatagaataaaagtatcagattagtgaatggttgtcctagtaatttggaagaaaaggaataaaaaatggagtcagactactaaatggcaaaaataaagacttgaagaacttaattttatacctgagctttgctttgattgaaagttgagaaatcttgtgatttgtcggtatcatcatctcaaatcaacttatttttcatctcaacatgaagaatatcagctctagccttttggaatttcagcaactcagatgcttgttagaacttatccaggactgcttgcaggacccaaaatactaataatccatatgcctttttttccagctgaaattagctgtcttaagagttgtttttcagtgctctgaggatatccacaaatatatcccaaggcactctgaagtgtgttttcaatctttggccctgcacactggggatgcccactggatgttgcatggagtgttgcctccagttgactttgatagacacaataaacaccacgtgagagccccctcatgaaggagggctgaggacttggagtccaattctccacctcccctgacagataggctattcaatgccttgggaatccgacatgaaggactgcttatagcagtccttgaaaactttctacagaatgtgaactgggagaagctaaatgctgtactcacaagctgggtgacttctccattgatttttaccatagctcactgaaacattttctaaattaacaggtataaattcacagacagatcagaatagatccttggtcctttccatttttctggcacttttgttgccaggaattcaaatacaactaagagaaaaagtgggtagctagcaagaacagatagtttctcaaatattagtggcagtaacccaactttgagaaaatatggagaaagagaagaaaaaacccttaagactgcagtgttagctgcccagttacaattattcacaaagtgagcaaatacttatttgggccaccaataaataagagggcacaagctaacgggaaattaatacgtcactcatgtaaaaagtcaacacattagaaaaaggaatcttggatgagattgagaaagatgcaagggataaaacctgacaagctttacaacctccttgaggaggtcttccctactctgtaaaatcagacagaaaatcattggcaaatggagcaaagcatgggcaaaactgatataatgaggaaacagagctgggaaaggataaatttatgtgtctgtttctgtttcctatcttttaatccttttttaccctttcttctggatactggagcaacgtattctgcaatttctacaggccttttctgttttatcctaaactaggaaaatatccagatataagtatttctggactatctgctacctcttctttctcctctgaggttttcatgcaaataggcgaggtcctatttgcataaaataggaagaatatgccttgtctcctaatatgcccaataatcttttaagaaagggcctgctgtgtaaaccaagggctatgatcttctgtactctgaagagagtgcttttggagcttctgggatgcaaggtatctacattgctaaatatgtgctttctgaaacaaaagatggctgtgggagagtaggagtctcactcaatgctggagaagcactcccaagttacagaaaataaacccatatctatgagtcctaggaacaaatgataaagatttaacaaaagagatcaataccattgttgtaacttacaacagagaaaaaccttggccatgtatagaacattttctctatttttacctcaaatatgatgtaattagaaaaattttaaaaaacgtggcatagttaaaaaaaaatggcacattcacttcataatgcttccatattgcctgtgggaaagttaggaaaacttgacagggaaggtcaaactgtttatagatttatgcatgatcttagagaaacaagttgtgattcctctgacacttgtaattcccagccttgccactgttttaatatctgttcctgagttatctaagtatttttctgtcatggatttatgttcaatgttttgttctccatgtttagctggggagtcgaggttcctgtttgagtttacctgtggagcgggaggctttcaatatttgtggcaacggatcttcctggaattcagagagtcacctactccattctctgggcatttataagaaaatataaagaatattcctcacattaggattccatactaattgttattgatagctgctaataacttacatgctagtaaagaagacattttagctttactaaatttcttagactcccaggacgtaaaacctcgctagctaagttgcaatattaccaactgaagtaaaatatttgggatctgtgttattggcagatggaaggtggttgcatcttgaagtcagcctaatatacaaatgaaacaacatgccaaaaaaaaacttcaccgattttgaggattaattgtagatctttgcgttttatgaaaaggctaagcctctcactgagatgttacatgaaacatcactagaactctttattaaagtcaaaataaggctgaagaagtcttttatgcccttaaaatagctgctatttcttcacttgtattacttttttaaccttgttaaaaaaggagttctaccttttctgtcttgagattaagggttactccaaaattaggccttgattataggcacattgcttactttcccaggttcttgtattttgtctctctggaaatgcgatgatatttataaggtacagtggcaaccactcccctgattaaaaaggctcattcagtccttgactctgagaaatttaacttacttacaggtacttcatgttgttactgctattttacaagttcataagacacaacacttgtcagtatgctgtcaaacgagctaagaacaagtcttattgtgtgatcctaatgctattctaggaaggtgtaaccttttgacagctattctgtggccagatcttgacagaataagttgaacttgactgtctcagagtgattgaggaagacactggagcatggtctaatttttgtaatattcctattcccaatgttgactgaatattcatgaatatattctgtgtgaaagacccacaaaggaatataaaagcatgttctacggtaatcaaccctcatgaagcttagaggcttctgtttttcgaaacataaaatcagcccaagtggcagaattgtagttattataccagctgttaaattagccaccagttataaagcagatatctatactggtaataactatatgtttgtggtctgttatgtgactagccaactttggaagaatagatgctaacgtcaataggaactaaaatattacatgagaagctaatttctgatttagtagagtcattcaaacttctctaacagatttatgtaatttattgtagagctcacagtggaaaacaagattaactctttaaaaagaaattaatttgcagacctagctgtgaagggcttctgacgtttagactggggaaatcaaacctctatcacttctgtccaaaacctcctattcataatttcagaaatagccaactctcttaagaattagaaaattaggaatgaaggagaactaagatgaacaccataggaaaatgagaattacataatgaaatctttcctttattccgattgttatatgattgagtggctttttcatgccaccaacaagttcgttcaagcaaaaggaatatgctgacaacttgaaatctttgtaatagcatcatataattcaggaaataatatgaagatatgctctttgtcagaaaaattctttccttacctaaaataagtcaagaaagcctactaaggcccatgatacctaggatctgctggcacaagaactttatagaacttccacctttagaagatagtaaatatttttaccagtgttgtgtattggtgtctggataggctaaagcagatgctcaaacagtgatcaagctcttactgaatcatatcatctcaaccactggaattcctgaatatctggaattcagtaggggaatcattctgtatttgtgggaattcgggaattttgtaatactctacaaataccaataatttacctacctgttatccccagtcttctgaacaagtggaatgagtgaaccacataccctaaagaattatttagctgaattatttagctaagttatgtaaaaccttaccatccgtttcatttaaaataagattaaatccctctagcaagcccaacattactccctttgagtttggaaggtctactaatcttgggcttaagcctaaccctttctctgatacaatggaaaatcctcatgaccatatatgatacttaaagagtttgtgggtttccctaggctcaccttgccaacatgtggccagatgctgaaaaagccgcctggggaagtgtgtcattgatattgaacaggagattcagtctatatcaaagtgttctgatgacagactgtgaccacactggacagaaccctatcaagtgtggttgagaattcatagtgctatcaagatgaaggagaaacccaagtggatacacgcttcacatgtgagaccaacatccaccatcgagtggggtataattcccatttaggaccttaaggataaattctctagatagtaaccttgaataatatgcaagctaaagagaaaaacttatttgatagacagcaggagagatagactgttttgttattaatacgttcttcttaattattttgaccattataattttgatacttagtttgtattaattttgatacttagtttgtatcaattgaacataaagcttattattagtctttcttttaaaagttagtatctcaacctcaaggaattttgtggtaattaaaaattgtcctgaatatgagatcaggacttaagaaacccctttctttgtcataatacttcaaactcaccaactggatgatttaaattcagaagacatgaaaaatttcaaatcttcccactgattagtcaatattacaaaaatcttaaatatcatcaccctttgtgttattgaagatactgatgagaagaaatttttaactaattaagttgaacgtaattatattcaaattgaaagtccctgaagggtatcacaaaactccaacaactctgtatcttttttttttttgagcagagtttcaccttattgcccaggctagagtgcaatggcacgatcttggctcaccacaacctctgcctcccaggttcaagcgattctcctgcctcagcctcctgagtagctgggattatagacatgcaccaccatgcctggctaattttgtattttttgtagagatggggtttctccatgttggtcaggatggtctcaaacttccaacctcaggtgatccacccgcctttgactcccaaagtgctgggattacaggtgtaagccaccacgcccggcccaactctagatcttaaatagccatgggggtccaatttaataaagcttaaatagacacacagatgagaaaatatcccagcccaaatggtgagggttagtttcttcaaaacccccaaaacgtatcatcacctaaaaatgagcttaccctgtttagaacctggcagtctccagtccccatacttacaaactcgaggataattaaatggtctgtatcatttaatattacaatagctaatgtgagtaaactgaattctacttgtacatgataggctttaccactatgaaatgtttttacacagggactaaaatggcactatgatcaaactgaaagagatttctttttttaatagatggggtctggctttgttgcctacactggagtgcagtagcacaatcatagctgtctatcgcctgtaactcctgggctcaagggcttcctccgtctcagcttcctgagtagctgggactacagggacatgccaccatgcctgcctatttttttttgtagagtcagggtcttgctcaggctggtttcgaacttctggcctcaagtgatcctgctgcctcggccttccgaagcactgggttcgcagacataagccaccatacctggccagagattattaaatacagtattagggggaataaaactaggactacaattgtaaactcttctgatatagaaacaattactgataaacagtgcattggacaatttgcagaaagatgcaaacatgatagaatcaaaatctaaacaagatatgacaaagaaaaacaaaaatggaattgggaaagcatttggaattggacatctggtaataaaattctattgcaaaaaaatatatagtaaagaattttcagaagccttcacttgcattagtgcacaaccactggttattcaacaactacaatgtatacttgctggtactgaagtacaagattatacctactcagacaaacaattaggtattggacatactactgtctatgtagagattaaatgatagtgtaaaaaggttgatcatattataggtaaaagagctgaaatctggcattgcagattgaaatttctgaaaactgaaataataaaactaatttcactaaagtatgtacattttcttatttacatagtttataatgtgttatataattcatgaatcttcaaaagaaatatactgagctattcaataaaaatttacattacagctttaggccaccaagccccatctattgattatgtacttggtgtaaattggcggggggaacaaaacaagagtgaaaactgagcctcaaaatacaaagcatgtagcaaaacagactactaaaattattctcaatttctaagcccattatatgatgcaggagaattcttatgtgtaggtaataaagaatctgttcaaatatgcatcagtggtatgacagtttcaaataacccactgaactaggaaatattcaccaccaaggaggtcaggtatactgatttggtcaacctggatattgacatgggatgttcttacggattgacagctccacttgatgctatcctatcacaaaatattattacctgatgttgatataattaacaagtcaatacaaaaagtaaggtctctctacaagacaagactaacatctctgtaatataaatgggactctgagtgtaatagatttagggtttctgcaagaattttgaaagttaaggatccaacgcctgtaatcccagcactttgggaggccgagacgggtggatcacgaggtcaagagattgagaccatcctggccaacatggtgaaaccccgtctctaataaaaatacaaaaaaaaaaaaaaaatagctggacatggtggcacacacctgtaatccccgctactcgggaggctgaggcaggagaatcgcgtgaaccagggaggcagaggttgcagtgagccgagatcgtaccactgcactctggcctggcaacagagcgagactctgtctcaaaaaaacaggaaagcaaaagaaaaagttaaggatcctaccaacaaaattggaatatgagactgaggaaattcctattccagctaaacccagaattgattatgttgatgaacacatgaatttcatgtgtgtgaaaacatatgaactcctcattgtcagtctaatgagagtgaaaacaacacatcgaagatgcggggagacgacaatacagagtgggctcataactgcattctagatactgggattactttccattaaggtatataattttagaatatctcacaaatagcccatgcaaaatcctgcggaaaggcaaccataaatatagagagaatatgtaaaaaagtggcatggtctagtatatgccaaacaaggcaagggtatacaaaaacctcaaggtcccagtatgacaaatcatgcttgtattgcacacccattaaaaagtcttctaaaaagtttttttaatggtgtgcaggcaggtttattcagaaaaagctcggattcatacttgtaaaagtctgagaaaagcattaaaatgctattatgatacagttgacccaaacaacacaggtttgagctgtgcaggtccacttatctgtggattttcttctacctctgccactgagacagaaagacaaactcctcctgctcctcagactactcagcatgaagatgaggatgaagacctttaggatgattcacttcttcctaatgaatagtgaatgtattacttccttaataacgtttttttttctctagcttcttgtaagaatacagtatataatatatgcaacacaaaaaatacgtgttaatcaactgctttatgttatcagtaaggcttccagtcaatgggagactaactactaattaagtttttggggagtcaagttatatgcagattctccactgagcaaatgttcagcactcttaactcctgtggtgttcaagtgtcaactgtatatatgtttaagagttattgtttacccagtagatcaagccaagaccaagttaggtttatgtgtttttggcttaacctaagaaaatgttcagactaaagcagtccatcaatagaacagactgccttttaaaccagggaacaacttgtctttgtaggtattttaggtatatgaatgaccatatgtgatctgctttgggtcatctatggtcccttccaacttaaaacttctctattcatgaacattgcataactgaattgtggatttcatataaataatatcttgttgcctactttaggtttgaaatgttttccttctcagttgcatagatatgtatagcaccatccccactatgaactctcatgtagaaaataccagattctaaagtcatctgtgaggctaaatgtcagacttaggcacttccacataatgctgcaaaaagcaacagcttaaaggtattacttgctcaacatgtcattccaaacacatacactatgattagcagctgaatttaacagtatctttccactttgaaaaacactttatatacctttacaaagaagccatagtatctcagggtagaaaaactcaacaggtagaaaacatggccttctcttagaaatgagctacgtaattttgagtacttatctcaaaatcattgcagaaacatatctagtgcttatggaaaaagaatcaaaagatctttttttttagctaaagattttgagtttttcttgtgacatttaagtcttttaatgtatgctgatttttatgattatctaactgtttcataatggttattcttgcctccatgaaagggaagactagaacaaaaacctgacaagaacaagttctagctccatcaccgacttactgagtgaccctaggcaaatcatgtgggctctttcttgtgttttaagttttacctccaatgaagtaactgcctcagactagctgtgaagctctcataaagtgatggatttgaaaatgccttgcaaagaatgagcagttaatacatagaaagtttcatgtacttccgtatattgcatgatgcccacccagggttaggttgataacagttgttgatccaataatgaaagaaataaaaagaactctttaaaataacgaagtaaaatagctaattttcctgttgtagttttaaaaattatataaactatataactaaacatatttatgtttattatatataatatgatatgcaattacataagacattattatataatgtttttatataatgcttaagtaattaaacaattatatttaaatataagttatactttccttccccagataagacaaagaacctgtttacttctctgagggatttattggttcctgtgaaatctgtggaggccgctaacactccatagccagagaatgacaacatatgattttcttttcagtcttgtagtatccacagtagtgatgtctgtccatgtacaagtgtctgtccaggacatccattaaattccatgcctgctctgtgtgttatgagggcagggacagaagacagattcactggtggtactttcaaacaatgtggtaggccttccttttggggtctgccatgacaatgataccccaggcggcaatgctaaggcccatgaccatcttcataatggtccatggacccggggggtatcccatcttcatgggcttctgtgccacccagtacacatacccgcctgcccccatcagccgcaacccagaaagcacgtgacagctccagcaggtctccaacaggtggtgttctgctggtgaggtcggcgatccgggtgtagctggggttgcaggcttggcaggcacggcggcggtatcgggaggcacagcgatcttgagggactcaaaaggctgggacaactgagaccccgtgttctggaactcggcctccctaaaaattatgttttaaaaaatacatagaatcggtaagatgaggaaaaagcataaaacggtcaaaaacactgggataattcaaggttcattttgacaatggtaatacaaatagaaaaactgacaaaaaagaaaatacatcttacattagaaaacatacaagagtagttgtatacaaacgaagacacgtaccaaaaataagttacaaaatatgaatgcatatacataaaaaatgtagtttcaaacaattcaaaagtaaaatgatgcaagacattaaaatattcacaaacacgctcgaaaaataagagctgacgcccaaattagaccagttcaacggaagtgtggggtgtggttggggatccgccgcagaaatcacgtggcgcccaggccaaggctgcaatttcccatgctgcttggagaagacgcctgggagggctcccaccatgcccaggggcaggctatgtgactgcccggtctgcagctgtaagtggtttctgggccagtctctttgtctggtcatgacctggttggtggccctagcttgtgccgagtggtcccggagtggcccagggggttgtgtgtggggagccagcgtgggcagcggaggtgctccctggggcctgcagtagggcacagcctctgcatcttgtgtgagcccagtgacccgcctgtcctgtccttccagggttgggtggcagggagggccaagggtgccctgcgtccgtgacgggtcagcgtttccagcaaatgggtccatggagggagagcctagctgagccggcgggctctaccaccttcatgaggttctgtttggacacctgtcctactcctgttcccccagatggtgaccatggaggaagacttgtgttgggcccaatggccctcggccagtgtccgtctgccaggtgccagcccccggtgcgctgaagctcaggccattcctgatgccctggcctcctgcactgagctgtggtgagcccatccgggtcctgctggatacatgggcggggaggggggtgccctgggttgtgttgatgatgagaaccttatattatcctgaagagaggtgatgacttaaaaatcatgctcaataggattacgctgaggcccagcctaggtgagaattttggaagaggatgctgggatccagaggcccccgacggggccactgtatttcgggctgcagacctagaggccctgaagggcatctgaaggtggcccaagcctatctctgagcaaccccgtgaggcagtcaaggatccctgtgtgggcataggctggggctctggttccctaggggcagggcatgtgggtggctcctccctgacccccattctcccctgtgtctttcagtgagctcttctgcccaggcaggccccctggcattgaccggcataggtgagtggatcctgctggggtcatgggccatgagccaggctgctggggtcgccaagggtcagcctttgggacaggagggtttccttagggagccaacctgaatccccactggggagggctaggttctccaggagggcagcttccttgggagtctgacccaaggaggatatcttctctaaagccctgcagtggtgagggtggtccaggcagcaaagggttccttcaaccccacccaggacagcgcccagctgagactcagtgtgcaggtctggccatgaggactgcccaggtcacataagggggtccataggagctccggggcaccacaggggtcacacaggagctctccttccccagagaggggtgagcgattgggcctatggggtcaggcctttggagatcaaggattgggccccacccatgaacctgaatgggactgagcctttgagcaatggaccatcccagtggtgcctcggagcctagtcgggggtgatgtggcttcaagggggttgggtcaaggctgagctctggccggtgcctggtagtgctgcaggctggccctgggactcgctggctttgggcaaaggggccagcccgtgtcccactggaggtcaccccttccctgtgggcaactagcaggaagggtggcatccttagctgggaaggcccactgtcgagggtcctgtccagggaatttgcctcccaggcttcttctgatccacggactcacctccagggtggtggggtcttgggcccgaggaaaggagaagataccctgtgttccccagttggctgagtgtggcttggctctgtgtttgggaggtgtgtagggtatgaatggggacccaccagagaacacacatggcccagggccccaggttatgatttcccatgcacacttggagagaactctgggtgagtgccctccactcccaggcacaggctatgcaactgccaggtctgcagtggcatgtggcttccaggtcagcctccttggttggccatgagcaggttggttgccctgtcctgtgcccagtggtcttggagtggcccaggggtcctgcaggggagctggtgtgggtagtggaggtgcttgtggggcctgcaatagagcgcagcctaggtgccttgcacgagctcagtgacccacctgccctgtcctagtcctagctcagtgacccacccccgctttgcagtgatggggagtaccaagcgtctcctgcgtctgtgatgggtcagtgttcccatcacatgggtccatggagggagagcatgcatgagctggcaccacccaccaccttcccactatggaggcgtgaggccatgtttggacacatgtcttcttgtccccccagacagtgagcctggaggaagacttgccctggtcccactgtccctgggccagtgtccatcagccaggtgcccagaccccagtgcgctgaagctcagccctccctggcactctggtctcctgcactgagctgtggtgagcacatccaggtcctgctggatgtttgcatggggaggggctttccccgggttgggtcgatgatgagaagcttctgttttcttgaagagaggtgatgacttaaaaatcatgctcaataggattatgctgaggcccagcctaggtgagaattttggaagaggatgctgggatccccggcaggagggattttgggctggagacctggaggccctgaagggtatctgcaggggccccatccctgtctctgcacttgtctgtgaggcagcccagtttccctgtgtgggcttcgtgtcagtgctctggttccctggagcagggcacgtgggtggctcatccctgaagtcacgttctccctttgtgtctttcagtgagctcttccgcccatgagggccccctggccttgagcagcataggtgagcggatcctgctgggctcatggaccaggccacatggggtcactgagggtcagccttcaggacaggagggattcctcagggagacaacctgaatccccactggtgagggattggttctccaggagaaccacctccttgggaatctgacccaaggaggacaccttccctgaagccctgcaggagtgacagtggtccaggcaggaaagggttccattagtcccatgcagggcagctcccagctgagacttggtgctagggtctggcctcgaggactgcccaggccacatgaggcagggtctcaggctgctatttccagtgcatgcttggagagggagcctgggtaggccccctccctgtgcaggggcaggctgtgcatctgctggggctgcagcggtgcctgccttcaggaccagcctccttggttggcttgagctggatagtggccttggccccacccaggggtcatggtgcagcccaggaattgcaggggagctggcatggggagtggaggtgctccaggggcttgcagtagggcacagcctgggcatcttgcttgagcccagtgaccagcctgccctgttttccagggttcagtggcagggaggaccaagggtcccccacgtccatgactatcactgtttcacatggatcccatcaggtgggtccatggagggagagcatgtgtgagtcagcaccccctgcctcctttcccgcatggaggcatgaggttctgttcggacacttttccctgtctctccagatggtgaccctgaaggaggacttgtgttgggcccaatggcctggggccagtgtctgtcagccaggtgcccagcccctggcgtgctgaagtttgggcccttcctggtgccctgttctatcgggggaacccgcccccaataattcaacgtaggtccttttctattttccctaagtgtcggccggtctgagaaataaagggaaagagtacaaaagaaagaaattttaaagctgggtgtccgggggagacatcacatgtcggcaggttccatgatgccacccaagctgcaaaaccagcaagtttttattagtgattttcaaaggggagggagtgtacaaatagggtgtgggtcacagaaatcacatgcttcacaaggcaataaaatatcagaaggcaaatgagggcagagcaccagggccaaattgaaattgctaatgaagtttcgggcacgcattgtcattgataacatcttatcaggagacagggtttgagagcagacaaccggtctgaccaaaatttattaggcaggaatttcctcgtcctaataggcctaggagcgcttcgggagaccagggcttatttcatcccttatctacaactgtataagacagacattcccagagcggccattttagagacctccccctaggaaggcattctctttctcagggttgttccttgctgagaaaaataattcagcaatatttctcctattcgcttttgtaagaagagaaatatggctctgttccgcccggctctcaggcagtcaggcctgatggttgtctcccttgttccctgaacatcgctgttatcctgttcttttttcaaggtgcccagatttcatattgtttaaacacacatgttttacgaacaatttgtgcagtgaacgcaatcatcacagggtcctgaggcgaaatacatcctcagcttacgaagatgacgggattaagagattaaagtaaagacaggcataggaaataataagagtattgatttgggaagtgataaatgtccatgaaatcttcacaatttgtgttcttctgccatggcttcagcaggtccctccgtttggggtccctgacttccctcaacactggtctcttccactgagccttggtgagcccatctaggtcctactggatgcatgtgtggggaggggggtgccctgggttgggtcaatgatgagaaccttatattgtcctgaagagaggtgatgacttaaaaatcatgcttagtaggattacgctgaggcctagcctaggtgagaatgttggaagaggatactggatgctgaggtccctggctgagctactgtattttgggatggagatctggtgactctaagggcacctgaagggccagaccctgtctctatgaggcagcccaggctccctgtgcaggcttgctgttggcactctggttccctggggtgggacatattggtggctcctccctgagccctgttctccccttgtgtctttcagtgagctcttccaccaaggagggccccctggcattgactggcataggtgagtggatcctgctggtgtcatgggccatgggccaggccacgtggggtcaccaagggtcagccttcaggaaagaagggtttcctcagggagccaaactgaatccccactgggaagagatgggttctccaggagggcggcttcctgggaaacaggaaccaaggaggacgccttccctgaagccctgcaggggtgacggtggtccaggcaggaaagggttccttcaattccacccagggcagctcccagctgagtctcggtgctcgcgtctggactgccctgactatgtggggggccacaggggctctggggtgccttgggggtcacacaggtgctctttgtccctggagaaaggtgggtgatcaggcccatggagttgagcagtggagattcaaggattgcacctgacacatggtcctgaatggaacccaccctgttgagcaatgggccatcccatgggcctcttcaagcctgaagaggggcggtgctgtttcactgggtttaggtcaaggctgagctctggccggtgcctggtggtgctgcagtctagctgttggactcgtgggctttgggccagtggggcagcctgcatccctctggaagtcaccccttccctgtggctggaagttaccccttcccagtgggcgaatgtggggaagggtcacctcctgaagtgagaaggcccactattgagggtcctgtcctggcaatttgctgcccatgctgcttctgagccatggagacacctccagtgtggtgggatcttgggcccaaggaagggagaagacactgtgccccccgattagctgagtgtggccaggctgtgtgtttatgaggtgtgaggggcatggatggaaactcaccagagaaagcatgtggccctgggaccccaggtcgtggtttccagtatgtgcccagatacagcacatgggtgggtgcccaccatgcacagggactggctatgtgactgccagctgtgcagcagcatcaggcatatgggccagcctccttggttgacaatgaactggttgctggccctggcctgtgcccagtagtcctggggtgaccctggtgtcatacaggggagctggcatgtgtagtggaggtgcttgtggggcttgcagtagggcacagccctggcttctagcctgagcccagtgacctgcctgtcctatcctttcggtgttcagtggcggagagagcattgggtgccctgcatccatgatgggtaagtattccacatggatctcatcacatgggtccatggaaggtgagcatccctgagctggcacaccacaccttctgtccacatggaggggtgaggttgtgtttgggcacttgtcctcttcctgttccttgagatggtgaccccagaggaggacatgcgttgggcccgctgtccctggccagtggccatcagccaagtgcctagcccctattgcactgaaactcgggcccttcctgtcaccttggtctcctgcgttgagcttgggtgagcccatccgggtgccactggatgcctaggcagggaggggggtgccctgggttgggtcgatgatgagaactttatattgttctgaagagaggtgatgacttaaaaatcatgctcaataggattacgctgaggcccagcctaggtgagaattttggaaggggacactagaatcccgaggtccccagcagggctgctgtattttaggctggatacctcgaggccctgaagggcacctcatggaggggcccaatcctgtctgtgctcctctattaggcacaccagactttctgtgtgggcttggtgtccacgctcagcttccctggggtgggacacgttggcggctcctccgtgagccccattatcctctgtgtctttcagtgagctcttccccccagcagacccctgggattgacgagcataggtgagtggagactgctggtgtcatgggccatgagccaagctgcgtggggtccccaagggtcagccctcgggccacctgggtttcctcaggagctgatctgaatcccaactggggatgtgttctccaggagggcagcttccttgggaatcagacataaggaggaggccttctctgaagccccgcaggggtggcagttgtccaggcaggaaagggtttcctcagccccacgcagggcagtgctcagctcagacttggtgcttgggtcaggcctctaacatggccaggccacatgaggggggcaacaagggctccggggagccaagggcatctcacgggggctctccaaccctggagaagggtgggtgattcggtccatggggtcaggccatggagattcatggattgcagcaaacccaggaccctcaatgggactgaccctataagcagtgggccctcccagtggcacctctttgagccatttgtggggcggcgctgtttctctgaggttgggtcaagactagctctggccagtgcctgatggtggtgcaatccactcctgggactcgtgggctctgggccaaggctgcagcccatgtcccgctgggggtcaccccttcattgtgggcgagtggcagtaatggcctcctgagttgggaaggcccgcagtggaaggacctttcctggaaatttgcagtccaggctgcttctcagccattaggacaactccagcgggttggggggcaggggcgggtcttaggcccaaagaaaggagaagataccatatgtctcccggttggctgagtgtggccctgctgtgtgtgtgggaggtgttgtgtggggcatggattgggacacaccagagaaagcttgtggccctggagccccaggctgcgatttccagtgcaccttgaggaaaggagggtgggtgccctccatgcacagggcagactgtgcaactgccaggtaggcaacagcacgtggtgtccgggccagggtccttggtttgctgtgagctctttggtgtccgtggcccctacccaaggatccagataccacccggggtcatgcaggggagctagcatgggcaatggaggtgctcaaggggcctgcagtagggcacagcccaggtgtcttgcctgagcccaggaacccacctgccctgtccttccatggttcagtggctgggagagcaaagggttccgctgatctatgacacatcagtgtttcccatggatcccatcacatgggtccacggagagagagcatgtgtgagctggcacccccaaccccattccctgtatggaggcgtgagcccatggttggacacatgtcctcctcctgtccccccagatggtgactgtggaagaagacttgctttgggaccgctggccccaggccagtggttgtcatccaggtgcccagcccttggtgcacttgagctcaagcccttcctggtgccctggcctcctgccctaagctgtggtgagcccatccaggtcccactggatgcatgcatggggaggagggtgccctgggttggatcgatgatgagaaccttatattgtcctgaagagaggtgatgacttaaaaatcatgctcaataggattacgctgaggcccagccttggtgagaattttggaagaggatgctgagatcccaaggtctctggcagggccactgtattttgggctggagacctggaggccctgaagggcatcttaggggtgcccgaccccatctctgcgctcctccttgaggcagcccaggctcccttgcgggcttggtgtcagccctccagttccctggggttgaggcacattggaggctccttcctgagcccccattctcccctgtgtctttcagcgagctcttctgcccagggggacccctggccttaagtgggataggtaagtggatcctgctggggtcgtggaccatgagccaggtcgctttgggtcactgagagtcacccttcaggccacgagggattcctcagggagcccatctgaaacccaccagggacggatggtttctccaggaggacagcttccttggaagtcggtcccaagaaggatgccttccctgaagccgtgcagagatgatggacgtccaagcaggaagggcttccttcaactcaatgcagtgcacatcctagctgagactcagtgcacaagtcaggcctggaggacttcccaggccacatgagggggtccacagggtctccaggggccacgggtgttatgagggtgctctctgtccccgaggcttgcagaggggcagggtgtgggtgatcggacttatggagtcaggccgtgaagattcaaggattgggcccaacccatggcccagcatgggccgaacctgtagagcaatgggccatcccggtggtgcatccttgagccttgtgggggtggtgtgaccttagtggggttgggtcaagcctgagctctggccttcacctggtggtgctgcaggctggccctgggaatttgggctttgggccaagggtgcagtctgtgtcccgctaaaggtcacccctccccaaagtggagtggccagaaggatggcatcctgtggtagaagtcccactgtcaagggtcctgtcctggcaatttgttgcccaggctgcttctaagccatgggttcacctccaaagtggcggggtcttgagccgaaggaaaggggaagacatcatgtgttccctgattgactgagcatggcctggctctgcatttggaggtgtgcggtgcacgaatggggacccatcagagaaggcatgtggccctggggctccaggatttgatttctggtgtgagcttttacagggggtctgtagtggcacgtagcattcaggtcagcctacttggtttgctctgagcttgttggtggtcctggccctgcccagtggtcccattgtggcatagagtcatgtgagggagctgtggcaaggagggccaagggttccccacattcatgatcgttactgtttacatggatcccatctcttgagtccatggagggacagcatgtgtgagccagcaccccctgcccactttcctgcatggaggcgtgaggccgtatttctatacgtgtcccccacccctccagatggtgaccccagaggaagacgtgcattgggtccgctggccccaggccagtgtctctcagccatgtgctcaacacctggtgagctgaagctcaggcccttcctggcatcctggtctcctgcactgagctgtggtgagcacatctgggttcctctggatacgtgtgcagggagggggatgcactgggtcgggtcaatgatgagaaccttatattgttctgaagagaggtgatgacttaaaaatcatgctcaataggattacgctgaggcccagcctaggtgagaattttggaagaggatgctgggatcccaagttccccagcatggccagtatattttaggcaggagaccctgaggctctgaacggcatctccggggggcacaaccctggctctgggctcctctgggaggcagcccaggttccctgtgcaggcttgggctctggttccctgagcccccattctccctcgtgtgtttcagtgagctcgtcctcctaggtggggcccccaggcattgaccggcataggtgagtggtcctgctggggtcatgggtcatcagcaggttggagtcaccaagggtcagcctttgagacaggagggtttccacaggcagccgacctgaatccacaccatggagggatagattggtcctccaagagggctgttttttttcggaatcagacccaaggaggataccttccctgcagccctgcggggatgacggtggtccaggcagggaagggttccttcagtctcacgcaggtcagctcccagctgagtctcagtgcattggtctggcctccaggactgaccaggccacgtgaggggggcaacaggggcttcgggatgcagtggggtcacacaggatctctccatccccagagaaaggtgggcatagaacccatggattcgagctgcagagattcaagaatcgcacacgaattattgccctgaatgggacccaccctgttgagcaatgtcccatcccagggcacctctttgagcctgaagggggtggtgctgtttccctaggtttgggtcaaggctgagctctggccagtgcctggtggtgctgtagtcctgtcatggactcgttagctttacgccaaggggtcagcccacgtcctactggggatctccccttccctgtggctggaagtcaccccttccccattggcaagtgtcaggaagggtgacctcctgaggtggaagtcctgttgagggtcttgtccctggaatatgccacgcatgctgcttctgagccatggggtcacctccagggtagcgggtcttgggcccaaggaaaggagaagtcaccatgtgtcccccgattagccaagtgcggccctgctctgtgtttaggaggtgtgtggggcatggatggggacccaccagagaaagcatgtggcccagggaccccaggttgcaatttctaggacatgctgggatagggcacctgtgtgggtgtccacagtgcacagggataggctatgtgacttgcaggtctgcagcggcatctggcatccaggccagcctccttggtgggccgtgaactgaatggtggcgctggcctgtgcccagtatatctggtatgaccctggggtcgtgtgggggagccggcatgggcagtggaggtgcttatgggacctgcagtaggacacagtcggggcttcttgcccaagcccagtgacccgcttgccctgtccttccagggttcggtggctgagagagcttttgttgccctgtgtccgtgacgggccagtgttctgcatgcatcccatcacatggatccatggagggtgagcatccctgggctgacacaccccaccttctgcccccatggaggggtgaggttgtgttttcacacctgtcttactcctgtttccccagatgttgagcccagaggaagacttgcactgggcccaatggccccaggtcagcgtccatcagccaggtgtccagcccctggtgcactgaagatcgggcccttcctggtgccctggtctcctgcattgagctgtggtgagcacatccggatcctgctggatgcgtgtgcggggaagggggtgccctgggttgggtcaatgagaaccttatattgtcctgaagagaggtgataacttaaaaatcatgctcaataataggattacgctgaggcccagtctaggtgagaggtttggaagaggatgctgggattccgaggtccccaggagggcccctgtatttttggctggagacctggaggccctgaagggcatcttgtgggtgcccaaccctgtctctgcgctcctccttgaggcagcccaggctccctgtgcaggcttggtgtcagccctccagttccctgggtttgaggcacatgggaagctcctttctgagccccgttctcctcttgtgtctttcagtgagctcttccacatggaagacccctgtcattgactggcataggtgagtggctggcatcatgggccatgagccaggccgcgtgaggtcgccaagggtcagccttcgggaaagaagggtttcctcagggagccaacctgaatccccactgggaagatatgggttctccaggagggcgacttcctgggaaacaagaaccaaggaggtccccttccctgaagccctgcaggggtgacggtggtccaggcaggaaagggttccttcagttccacccagggcagctcccagctgaggctcggtgcttgcgtctggacttaaggactgccctggccatatgaggggtgccacagtggctctggggtgccatggggtcacacaggcactctctgtccctggagaaaggtgggtgattgggcccatggagttgagctgttgagatgcaagttttgcacctgacacatggccctgaatgggacccaccctgttgagcaatgggccatcccatgggcctcttcgagcctgaaggggggcggtgctgtttcactgggtttaggtcaaagctgacctctggccgccacctggtggtgctgcagtccagcccttggactcgtgggctttgggccaagggggcagcctgcatcccactgaaagtcaccccttccctgtggctggaagttatcccttcctcatgggcgagtgttgggaagggtggcctcctgaagtgggaaggcccgctattgagggtctgctcctggcaatttgctgcccatgctgcttctgagccatggagtcaccaccagtgtggtggggtcttggacccgaagaaaggagaagacactgtgccccccgattagctgagtgtggccaggctgtgtgtttgcgaggtgtgaagggcatggatggagactcaccagagaaagcatgtggccctggggccccaggttgtggtttccagtatgtgcttggatatggcatatgggtgggtgcctaccatgcacagggactggctatgtgactgccagctgtgcagcagcatcaggcatacgggccagcctccttggttgaccatgaactggttgctggccctggcctgtgcccagtagtcctggggtgaccctggtgtcatgtaggggagctggcatgtgtagtggaggtgctcgtgtggtctgcagtagggagcagccctggcttctagcctgagcccagtgacctgcctgtcctgtcctttcagggttcagtggcggagagagcatcaggtggcccacgtctgtgatgggccagtattccacatggatctcatcacatgggtccatggaaggcgagcatccctgagctggcacaccccaccttctgccagcatggaagggtgaggttgcatttggcacctgtcctcttcctgttcctcgagatggtgaccccagaggaggacttgcattgggcccactgtccccgggccagtggccatcagccgggtgcctagcccctgttgcactgaagctcaggcccttcctgttgccctggtctcctgcattgagcttgggtgagcccatccggttgccactggatgcctaagcagggaggggggttccctgggttgggtcaatgatgagaaccttacattgttctgaagagagatgatgacttaaaaatcatgctcaataggattacgctgaggcccagcctaggtgagaattttggaagaggacgctgggatccctaggtccctgacagggccactgtattttgggctggagacctggagtccttgaagggcatctggagggggcccaaacctgtctctacgttcctccatgaggcagtccaggctccctgtgcgagcttcgtgtcggtgctcagattccccttggtggtgcatgtgggtggctcatccctgagcccctgttctccccttgtgtctttcagtgaactcgtttgcccaggaggacccctggccttgagcagcataggtcagtggatcctactggggtcatgggccatggaccaggccacgtggggtcactgagggtcagctatcaggacaggagggattcctcagggagacaacctgaatccccactggtgagggattggttctccagaagggccgcctccttgggaatctgacccaaggaggacaccttccctgaagtgctacaggagtgacattggtccaggcaggaaagggttccattagtcccatgcaggtcagctcccagctgagacttggtgctagggtctggccttgaggactgcccaagccacatgaaggtggccccaggctgtgatttttggtgcgtgcttggagagggagtctgggcaccctccctgcacaggggcaggctgtgcgtctgccaggtctacagtggcgcctagcttcagagcaagcttccttggttggccttagctggttggtggccctggctcctacccaggggtcacaatgcagcccaggaagtgcagggaagccggcttggacagtggaggtgctccaggggcctgcggtagggcacagcctcggcatcttgcctgagcctagttactcgcctgtcctgtccttccagggtttggtggtggggagatacaagggttccccaggtccataactgtcctcgtttcacatggatcccatcacatgagtccacggatgtagagcatgtgtagccagcagcccctccccgctttcctgcatggaatcttaaggccctatttggacacctttccccgtccctccagatggtgagcacagaggaagaattgcgttaggccctttggccccggaccagtgtctgccagccacgtgctcagcctcccgtgcgctaaagctcaggtccttcctgtcatcctggtatcctgcactgaggtgtggtgagtccatccaggtccctctggatgtgtgagtggggatgggggtgtcctgggttgggtcgatgatgagaaccttatattgtcctgaagagaggtgatgacttaaaaatcatgctcaataggattacgctgaggcccaacttaggtgagaattttgaaagaggatgctgggaatcacaaggtccccggcaggaccactgtattttaggcgggagacctcgaggccctgaagggcatatcatggaggggtacccaacccagtctgtgctcctccattaggcacaccaggcttcctgtgtgggcttggtgtctgcactcagcttccccgggggtgggacacattggtggctccttcctgagtcgcattctcctctgtgtctttcagtgagctcttccccccagcaggccccctgggattgactggcaaaggtgagtggatactgctcgtgtcatgggctgtgagccaagctgcgtggggtccccaagggtcagccctcgggtcacctgggtttcctcaggagctgatctgaatcctgactggggatgtgttctccaggagggcagcttccttgggaatctgacacaaggaggaggccttctctggagccctgcaggggtggtggtggtccaggcaggaaagggtttcctcagctccacacaggacagtggagctgggcagtgctcagctcagacttggtgctcaggtcaggcctctaggatggccaggccacatgacgggggccacaagggctccagggcaccacagacatctcatgggggctctccaaccctggagaagggtgggcaatttggcccattgggtcgggtcatggagcttcaaggattgcaccaaacccatatccctcaatgggaccgaccctattaagcaatgggccatcccagtggcacctctttgagccatgtggtgggtggcgctgtttctctgaggttgggtcaagactagctccggccagtgcctgatggtggtgcagtccagtcctgggactcgtgggctttgggccaagggtgcagcccgtgtcctactggggatcaccccttccttatgagggagtggtgggaagggtatcctcctgaggtaggaaggcccgctgtggaaggaactttcctggaaatttgcagtccagactgcttctgagccatgaggtcatctccggggtggcggggtcttgggcccaaggaaaggagaagacaccatatgtctcccgattggttgagtatggctccactctgtgtgtgggaggtgttgtgtggggcatggattgggacccaccagagaaagctcgtggcactggaaccccaagctgcaatttccagtgcaccttgggaaggggactgggtgctcaccctccatgcacagggcaggctgtgcagctgccaagttggcagtggtgcgtggcatccgggccagcgtccttggttcgctgtgagctggttggtgtccttgacgcctgcccaggggtcccagtgctgcttggggtcatgaggggagccagcgtgggcaagggaggcctgcagtagggcacagcccgggggtcttgcctgagcccaggatcctgccggccctgtccttccatggttcggtggctgggagggcaaagggtcccccacgtctgagacacatcagtgtttcacatggatctcatcacatgggtccatagagagagagcaagcgtgagctgacaccccccacccccttccctgtatagaggtctgagcccctggttggacacatgtcttcctcctgtccccccagatggtgtctgtggaggaagacttgccattgggttgctggccccaggccagtggttgtcatccaggtgcccagccttggtgcactgaagctcaggcccttcctggtgctctggtctcctgcactgagctgtggtgagcacatccgggtcccactggatgggtgtgtgtggtggggggtgccctgggttgggtcgatgatgagaaccttatattgtctgaagagaggtgatgacttaaaaatcatgctcaataggattacgctgaggcccagcctaggtgagaattttggaagaggatgctgggaattacaagttccctgtgaagtccactgtattttgagctagagacctggaggctctgagggcatctggagggtgcccaaccctgtctctgcactcctccatgaggcagcccaggctcactctgtgggcttggtgtcggcgctcctgttacccaagggtggggtactttggcatctcctccctgagcccccattctcccctgtgtctttcagtgacctcttttgcccggcagaccccctggcattgaccagcataggtgagtggatcctgctggggtcatgggtcatgagccaggctgtgtggggttgccgagcatcagcctttggctgcaagggtttcctcaggaactgacccgaatgcccacctgggatgggttctccaggagggcagcttccttaggaatcggacccaaggaggataccttccatgaagccctgcaggggttgtggtggtccaggcagtaaagggtttcctcagccccatgcagggcagcacccagctgaaactcggtgcttaagtcaggcctctaggatggcccgtccacatgaggggaaccacaagggcactggggcgccaagggggtcttatgggggctctccgaccttggagaggtatgtgtgattcggcccatgaggtcaggttttggattttcaaggatctctccaaacccatggccctgaatgggaccgaccctattaagctatgggccatcccagtggtgcctctttgagccaagtgtggggtggcactgtttccatgaggttgggtcaagactagctctggtgaccccatgatggtgctgcagtctggccctgggactcgtgggctttgggccaagggtatagcccatgtcccaccaggggccacaccttccttgtgggcaagtggcggacagggtgtcctcctgaggtgagaaggcctgctgtggaaggacctgtccaggctgcttccaagctatggggtcacctacagggtggtggggtcaaggaaaggagaagacaccatgtgtctcccgattggctgagtgtggccccactctgtgtgtgggaggtgttgtgtgaggcatggatggggacatatcagagaacacacgaggccctgggtccccaggctgcaatttccggtgtggtgtggagcagggcctgggtgcttgccctccacacacaggggcaggctgtgctactgccagattggcagcagtacgtggcatccaggccagcctccttggttggccgtgagacggtaggtggccctggctgacacccaggggccagtacagccctggggtcatgtgaggcaactggagtgggtagtggaggtgctcaaggtgcctgcagtagggcatagccagggcgtcttgcctgagcccagtgacctacctgccctgtccttccagggttcagtggtgcagagggccaaagttcccctgtgtccatgacacatcagtgtttcacatggatcccatcacatgggtccatggtgggagagcaatcgtgagctagcatcccccacccccttcctgcatggaggcgtgaggttccatttggacacatgtccacctcctgtcccccagatagtgagactggaagaaaactttcattgggcccgctgtcccggggtgagtggtcatcagtcaggtgcccagcccttggtgtggtgaagctcaggctcttcctggcgccctggtctcctgcacggagctgcggtgagcacatccgggtcccgatggatgcatgcgtggggatggagggcgtgccctgagttgggtcaatgatgagaaccttatattgtcctgaagagaggtgatgacttaaaaatcatgctcaataggattacgctgaggcccagcttaggtgagaattctggaagaggatgctgagatccgaagtcccctggaagggccactgtattttgggctggagtccttgaggccctgaaggacaccttcggggtgcccaacccagtctctgcactcctctgtgtggcagccaaggctccttgtgcaggcttggtgttggcattctggttccctgggggtggagcacatcgacggctcctccctgagcccccgttcttctctgtgtctttcagtgagctcttctacccaggcgggcccctgcccttgagcggcataggtgagtggatcttgctggggtcacgggccaagagccaggccacggggggtcgctgagggtcacctttcaggccacgagggtttccttagggagctgacctgaatccccaccagagatggatggtttctccggaagggcagcttccttgttaattgatcccaagaaggacgccttccctgaaaccctgcaggggtaatggaggcccaggcagcagagagttccttcaacccaatacagtgcagcagcttgttgccttgatgctccccctccctggagaaaggtgggcatagggcccgtggagttgagttgcagagatttaaggatcgcacaggacttattgacctaaatgggactgaccctgttgagcaacgtgcacttggtgcatgggtcaggtgtggagcactgcccaggccacatgaggggtccactgggtctctgggggccacgggtgttatgcaggagctctccgtccccaaggcttgcaggggggtaggatgtcggtgatcgagcttatgcagtcaggccgtgaagattcaaggattgggcccaacccatggccctgatgggaccgaccctgttgagcaatgggctatcccactggtgcctccttgagcctggtgggggcagtgcggcctcagtgggcttggatcaaggctgagctctggccagcacctggtgtttggccctgggaatcgtgggctttgggccaagggttcagcctgtgtcccactggagttcatccctaccggaagtggagtggatggaagggtggcatcctgtggtggaagacccaccgttaagggtcctgtcctggcaatttgatgcccaggctacttctgagccatggagtcacctccagggtagcagggtcttgagccaaaggaaagttgaggacatcatgtgtcctctgattggccgagcgtggcccagctctgtgtttaggaggtgtgcggtgcatgaatggggacccaccagagaaggcatgtggccctggggccccaggatgtgatttctggtgtgtgcttataaagggtgcctggatgggttcatttaatgcccaggggcaggccatgtgactgccaggtctgcagtggtacgtggcatccaggccagcctccttgatttgctgtgagctatttggtgcccatggccctgcccagtggtcctgatgcagcccagattcgagcaggggatttggcgtgggcagtggaggtgctccagggtcctgcactagggtgcagcctgggcgttttgctcgaaccctgtgaccctcctgccccgtccttccagggttcagtggccaagggttcctcgcgtaaatgaccatcactgttttacatggatctcatctcatgagtccatggagggagagcatgtgtgagccagcaccccctgcccactttcctgcatggaggcatgaggccttgtttggacacctgtcccccacccttccagatggtgaccccagaggaagacttgcgttgggcccactggctctgggctcagcccctggtgagctgaagctcaggcccttcctggcaccctggtctgctgcactgagctgcggtgagcctatctgggtcccactggatgcatgggttgcggggcagtggagggtgatgccctgggttgggtcgatgatgagaaccttatattgtcctgaagagaggtgatgacttaaaaatcatgctcaataggattacgctgaggcccagcctaggtgagaattttggaagaaggtgctgggatccggagttccctggaatgaccaatgtattttgggctggagaccttgaggccctgaacagcagcatctcgtgggggcccaaccccttctccgtgctcctctgtgaggcagcccaggctccctgtgtgggcttggtgttagtgctccggttctgtgaacccccattctccccttgtgtctttcagtgagctcttccacccaggtgcaaccccctggcattgaccagcataggtgagtggtcccgctggggtcatgggtcatcagtcatgcagtgtggggtcaccaagagtcagcctttgagacaggagggtttccacaggcagctgacctgaatccacaccatggaagggtaggttggtcctccaagagggctgtttttttgggaatcggacccaaggatgatatctttcctgcagccctgcagtgatgacggtggtccaggcaggaaaagtttccttcaatctcatgcagggcagctcccagctgagtctcagtgcactggtctggcctcgaggactgaccaggccacgtgaggggggccacaggggctttgggatgccatggggtcacacaagagctccccatccctggagaaaggtgggcatagagcccatggagttgagctgcagatatttaaggatcacacaggatttatagccctgaatgggaccgaccctgttgagcaatgtgccatccatcctatggtacctctttgggcctggatggggtgttgctgtttccctaggttttggtcaaggctgagctctggccagtgcctggtggtgctacagtccagccacggacttgttagctttaggccaagggatcagtccacgtcctgctggggatctccccttccctgtggctggaagtcaccccttccccattggcaagtgtgaggaagggtggcctcctgaggtggaaggcctgctgttgagggtcttgtccctggaatatgccacccacactgcttctgagccatagggtcacctccaggatagtgggtattgggccaaaggaaagaagtcaccatgtgtcccctgattagccaagtgtggccctgctctgtgtttaggaggtgtgtggggcatggatggggacccaccagagaaagcatgtggccctgggactccaggttgtgatttccagttcatgctgggatagggcacctggatgggtgcccacaatgcaccggggcaggctatgtgactgccagctttgcagcggcatctggcatccgggccagcctccttggttggccatgaactggttggtggccctggcctgtgcccagtagctctgtcgtaatcctggggtcatgcgagggagccggcatgggcagtggaggtgctcgtgggacctgcggtaggacacagccaggtcttcttgcccgagcccagtgacccacctgccctgtccttccagggttcagtggttgagagtacagggtgacttgtgtctgtgacaagccagtgttctgcatggatcccatcacgtggatccatggacggcaagcatccctgagccgccacaccccaccttctgcctccatggaggggtgaggttgtgtgtggacgcctgtcctactcctgtttccccaggtattgagcctggaggaagacttgaattggtcccaatggtcccaggccagcatccatcagctaggtgctgagcccctggtgcactgaagattgggcacttcctggcaccctggtgtgctgcaccgagctgtgatgagcacatccgggtcctgctggatgcatgcgccgggaaggacgtgccctgagttgggtcgatgatgagaaccttatattatcctgaagagaggtgatgacttaaaaatcatgctcaataggattacgctgaggcccagtctaggtgagagttttggaagaggatgctgggatcctgaggtcccctggtagagccaattgtattttgggctggagacctggaagccctgaagggcatctgggaggcccaaccctgtctctgtgctcctccatgaggcagccctggctccccatgtaggcttggtgatggccttcctgttccctgggggtggggcacatgggtagctcctccctgacccccattctctcctgtgtctttcagtgagctcttccccacaggtgggccccctggcattgactagcatcggtgagtggatcctgctggggtcgtgggccgtgcaccaggctgcatggggtcaccaagtattagccttcaggacaggagggtttcctcagggagtcgaccttaatcctcacctgtggagagatgggtttgtcctctaggagggcagcttcctgggaaatcagacccaagacgatgccttccctgaagccctgcaggggtgacggtggtccagtctggaaagggttccttcagtctcatgcagggcagctcccagctgacacttggtgtgagagtctgtccttgaggactgcccaggacaaatgagtgggacaacagaggctcccggataccacagggatcactcaggggctctccgtccctggaaatgggtgggcgatcgggcccatggggtggggctgcggagattcaaggatcgtgccctgctcatggccctgaatgcaaatgaccctgttgagcaatgggtcacaccagtagctcctttttgagcctggtgggtggcggctcagcttcaactggttgggtcaaggctgagccatgactggtgctttgtggtgccgcaggccaaccctgggactcatgggctttgggtcaagggtgcagcccgcctgctgctgggggtcaccccttcttcgaggtcagtggcgggaagggtcacgtcccgaggttggaaagccccctcatgagggacctgtcctgggaatttgttgcccaggctgcttctgagccatgtggtcacttccgggtggcggggtcttgggcccaaggaaaggagagggcaccatgtggctcctgattggctcagtgtggcccagctctgtgtttgacaggggtctggggtgtggatgaggacccaccagacaaagcacgtggccctgggcccaggctgtgatttctggtgcgtgcttggagaaggatcctgggcaggcattctctccaaacaggggcaggttgtgggactgccagattggcagcagtgcctggcatccaggccagcatccttggttggctgtgagctggttggtgaccttggaccctgcccagggtttccgttgtggtccagagtcatgcagtggggccagtgtgagcagtggaggtgctccaggggcctgctgtatggcacagccagggcatcttacccaagccctcctgcactggtctcctgcactgagctgtggtgagcccatctgggttcctgtgatgcctgtgcggggagggggttgccctgggttgggtcgatgatgagaaacttatattgtctgaagagaggtgatgacttaaaaatcatgctcaataggattacgctgaggcccagcctaggtgagaattttggaagaggatgctgggaattatgagttccctggcagaaccactgtattttgggctggagacctggaggctctgagggcatctggagggtgcccaaccctgtctctgcgctcctccgtgaggcagcccaggctcactatgtgggcttggtgttggcactcctgttacccgggggtggggcattttggcatctcctttctgagcccccaatctcccctgtgtctttcagtgagctcttctgcccagcagcctccctggcagtgaccagcataggtgagtggatcctgctggggtaatggcccatgagccaggctgtgtggggtcaccaagcatcagccttcagccataagagtttcctcaggagctgacccgaatgcccacctgggatgggttctccagaagagtggcttccttggaaatcatacccaaggaagataccttccctgaagccctgcaggggttgcagtggtccaggcagtaaagggttttctcaacctcatgcagggcagtgccccactaagactcagtgctctggtcaggcctttaggatggccaggccacatgagaggggccacaagggctctggggcaccaaggggttcttgcaggggctcttcaaccttggagaggtgtgtgtaattcagcccatgaggtcaggctgtggattttcaaggattgcaccaaacccatggccctgaatgggacaaaccctaataagttatgggccatcccagtggtgcctctttgagccaagtgtggggtggcactgtttcctaagggtgagtcaagactagctctggctggcacctgatggtggtgcaatcctgccctgggattggtgggctttgggacatgggtacagcccatgtcccattggggtcaccccttcattgtggacaggtggcgggaagcatgtcctcctgaggtgggaaggcctgctgtggaagaccctgtctcagaaatttgctgtcttggctgcttctgagctatggcgtcacctctgtggtggcgggagtctcgggtgaatggatcctgctggggtcatgggccatgagccaggccggggtggggtccctgagcgtcacctttcgggtcatgagggtttcctaagggagccgacctgaatccccaccagggatgaatggtttctctgggaagcaggtttccttggaaatcagtcccaagaaggacaccttccctgaaaccttgcaggggtaacagaggtccaggcaagagagggttctttcaacccaacgcagtgcagatcctagctgagacttggtgcacaggtgagtcctggaggactgcccaggccacatgagggcatccacagggtctccgtggccaggagtcttatacgggagccctctgtccccgaggcttgcagaggggttgggtgtgggtgatctggcttacggagtcaggccgtgaacattcaagggtcgggaaccacccatggccctgaatgggaccgaccctgttgagcaatggccatcctggtggtggcaccttgagcctggtgggggtggcgctgcctcagtggggttgggtcaaggctgagctctgcctgtcaccttgtggttggccctgggaatcatgggctttgggccaagggtgcagcctgcgtcccgcttgaggtcactcctacctgaagtggagtggccggaagggtggcatcctgtggtgggagacccacggtcaaggatcctgtgctggcaatttgctgcgcagcctggttctgagtcatggggtcacctccagggtgacagggtctttgtcaaagggaaggagaggacattatgtggcccctgattggccaagcatggcccagctctgtgtttgggaggtatgcggtgcctgaatggggacccaccagagaaggcacatggccctggggccccaggatggtatttctggtgtgagcttataaagggtgcatggatgggtgcattccatgcccagtggcgggttatgtgactgccaggtctgcagtggcacatggggtccgggccagcctccttggtttgccatgagctggttggtggcccaggccctgcctagtggtctcattgcagcccagagtcatgtgggggagctggcgtgggcagtcgaggtgctccagggtcctacagtagggtgcagcctgggcgtcttgctggaatcccatgacccacctgccctatccttccagggttgagtggcagggagggccaagggttcctcgtgtgcatgactgtcactgttttacatggatctcatctcatgagtccatggtgggagagcatgtgtgagccagcacaccctacccactttgccctatggaggcacaaggccctgtttggacacctgtcccccacccctccagatgctgaccctggaggaagaattgcactgggcccactggccccgggaaaatgtccctcagccaggtgcttagcccctggtaatctgaagctcaggcccttcctgatgccctggtctcctgcactgagctgtggtgagcacatctgggtcccaatggatgcatgcatgcgttgtggggtggtggtgtcctgggttgggtcgatgatgagaaccttatatgttctgaagagaggtgatgacttaaaaatcatgctcaataggattacgctgaggcccagcctaggtgagaattttggaagaggatgctgggatttctaggtccctgtcatgggcactgtattttggactggagacctccgggccctgaacagcatctcgggggggactctaccctgtctccacattacatgtgaggcagcccaggctccctttgtgggcttggtgtctgcactctggttccctgtggtagggcagcttgctgggtccttcctgagcccctgttctccctttgtgtcttctgtctttcagtgagctcttcagcccaggtgggctccctgacactgatgcgcataggtgagtggattctgctggcatcatgggccatgagccaagctgcgtagggtcaccaaaggtcactctttaggccacgtgggtttcctcagggagccgacctgaatccccaccaggttgggacagtttctccaggaggctggcttccttgggaatcagacccaagacaatgccttccctgaagccttacaggggtaacagttgtccagacaggaaagggtcccctcagtcccattgaaggcagctcccagctgagactcagtgtgcgagtctggcctcaaggatggcccaggccacatgaaggaggccacaggggctctggggtgccacaggggtcacacgagagctctctttccctggagtggggtgctcgaatgagcccatggggtcaggctgcagagattcaaggatcgcatcccaaccgtggccctaaatgcgaccgaccatgttgagtaatgggccatctcagtggggcctctttgagcctggtggggggcggggctgtttccctgggtttgggtcaaggctgagctctggccagcgcctggtgatgctgcaggccagcccaggttcttgtgggcgttggtccattcggcagccctgtcctcctggaggtcaccccttgcctattggtgattggctggatcctgaggtggaaagacctgctgttgatggtcctgtcccaggaatttaccacccaggctgcttctgagccatggggtcacctccagggtggcggagtcttggggctaagaaaaaagaggacactatgtagcccacggttggccaagggtggcctgactctgtgtttgggacatgtgcggggcatggatagggacccgccagagaaagcacgtggcccggggactccaggcttcgatttcaggtgtggcttgcaaatagcacctaggcaggtgcccaccacgccctggggcaggctatgcaacttccatctccacagtggcgcatggcttctgggccattctccttggttggccatgagctgattggttcccctggcctgtgccctgtggtcccggagtggctctgggatcgtgtggggcagccagcatggacagtggagatgctatcaaggccttcagtagggtgcagcccaggtttcttgcctgaacccagtgacccacctaccctgtgttttcagggtttgctgacagggaaggccaaacgtgccccacgtccatgctaggtcagtgttcctcatggaccccatcacatgggtatcccttaccccttccccacatggaagtatgaggctctgtttggacacatgtcctcttcctgtccccccagatggtgagcctggaggaagacttgcattgggcccaatggacccaggccagtggccatcagccaggtgcccaccctggtgtgctgaagcgcgggacctacctgttgtcctggtttcctacactgagctgaggtgagcacatctgggtccctctggatgcatgcatggggcggggggtgccctggtttgggtcaatgatgagaaccttatattatcctgaagagaggtgatgacttaaaaatcatgctcaataggattacgctgaggcccagcctaggtgagaatttggaagaggatgctgggatcccaaagtcccagtaggggactttatttatttattttgggctggagccctggaggccctgaagggcatctggggggcccagccctgtctctgtgctcctccgtgaggcagcccagcctccctgtgtgggcttgctgttggtggtccagttccttggggcggggcacgtggacagctcctccttgagcccccctgctccacttgtgtctttcagtgagctcttcaacccaggcggggcaccctggcattcaccagcataggtgagtggatcctgctgccatcgtgggccatgggccaggctgggtagagtcactgagggtcagccttcgggacaggagagtttcctcagggagcgaacctgaatccccaccagggagggataggttggtcctccagaagggtggcttcctgagaaatcggacccaagggggacgccttccctggagccctgcagtgatgacggtggtccaggcaggagagggttccttcagtctcacacatagtagctcccagctgagtctaggtgttcgagtctggccttgaggactgcccaggccacctgaggggccaacagggactctgggacaccatgggggtcacataggagctctccattcatggagaaagatggatgatagggcccatggagtcgagctgtggagatttaaggattgcgcccaatgtatggccctgaatgggacagaccctgttgagtaatgagccattctatgggcctcatcgagccttgagcggggcggtgctgtttccctatgtttgagtcaaggctggactctggccggtgcctggtggtgctgaggtctggccatggactcgttggctttgggccaaggggtcagcctgctccccctggaagtccccccttccccgtggctggaagtcatcccttcccaaggggcaagtgtcgggaatggttgcctcctgaggttgcaaggcctgctatctagggtcttgtctttggaatatgctgcccatgctgcttctgagtcatggggtcacctccagggtagcggatcttgggcccagggacagaagatgccgtgtgtcccctgattagctgattgtagccctgctctgtgttcaggaggtgtgtggggcatggatgcagacccaccagagaaaactgtggcccttagaccccaggttgcaatttccattatgtgctgggatagggcatctgggtgggcacccactatgcacagagataggctatgtgactgccagggctgtagcggcatctgacatctaggccagcttccttggtttcctgcgaatgagttggtggccctggcctgtgcctagtagttctggggtgaccccgggtcgtgtgggggagccagcaggggcccagtgaccagcctgccctgtccttccagagttctgtggctaagagagcactgggtaccctgtgtccgtgatgggccagtgttccgcatgcaaacgatcacatgggtccatggagggcaagcatacttgaactgccacaccccaccttctgctcacatacaggagtgaggttgtgtttggacagctgtcctactcctgttcccctagactgtgaaatgggaggaagaattgtgttgggcccaatggccctgggccagtatccgtcagcctggtgtccagcccctggtgtgctgaagctcatgcccttcctggtgccctagtttcctgcactgagcttggtgagcccatttgggtactgctggatgcatgggtggggagggaggtgccctgggttgggtcgatgatgagaaccttatattgtcctgaagagaggtgatgacttaaaaatcattctcaaaaggattatgctgaggcccaccctaggtgacaattttggaagaggacactggaaatcaatgaggtccctggcagggccactataatttggggtggagacctggaagccctgcagggcacctcgtgggggcccaaccctgtctcttcactcccctgtgaggcagcccaagctccctctgtaggcttggtgtcggcactctgctttcctggaggcggggcattttggctgctccttcctgagcccctgttctcccctgtgtctttcagtgagctcttttgcccagaagactccctggcattgatcggcataggtgagtgtctcctgcttgggtcatgggccatgagccagaccatagggttgccgagggtcagcatttgagccatgagggtttcctcaggaggcgacctgaatccccacctgggatgggttctccaggagggcagcttccttgtgaatctgagccaagtaagatgccttctctgaagccctgcaggggtgccagttgtgcaggcaagaaagggttccctcagccccaggcagggcagcgcccagcagagactcggtggttgggttaggcctctaggatgaccaggccacatgaggggggccacaagggctctggggcacaacaggggcttcgcagggtctctgcgaccccaaagagtggtgggcgattcggcccatgggatcgggccccagattcaaagatcacaccacacccatggccctgaatggaaccgaccctgttaagcattggggcatcccaggggtgccactttgaacctggtgtggggtgccgctgtttgcctgaggttgggtcgagactagctccggccagcacctgatggtggtgcagtccggccctgagattagtgggctgtgggcccagggtgcagcctgtgtcccgctgggggtcacctcttccttgtgggcgagtggtggaagggcatcctcctgaagtgggaaggcccgctgtggaaggaactgtcctggaaatttgccatccaggctgcttctgagccatggggtcgcctccagagtggcagggtcttgggcccaaggaaaggagaggacactgtgtttccctcaattagctgagtgtggccctgctctctgtgtgggaggtgttgtgtggggcatggatagggacccaccagagacaacacatggccctcgggccccaggctgcaatttctcagtgcatgcttggagacggtcctgggcctcctccatgcccagggtcaggctgtgcaactgtcaggttagcggcggcatgtggtgtctgggcctgtctacttcgttgtccgtgagctgctggtggccctagccctttcccaggggttcaggtgcagccctggggttgtgtgggacagccagtgtgggcagtggagctgctctggaggcctgctttagggtgcagcctgggtgtcttgcccgagtgtagtgacccgcctgccgtgtcctcacggggttccctagcaggaagggccaagggtcccctgtgtctgtgacacgtgagtgtttcccatgggtcccatcaaatgggtccacggagggagagcatgagtgaactggtaccccccacccccaccctcttccccacctggaggcatgagcccctggttggacatatgtcctcctcctgtacccccagatggcagcgctggaggaggacttgtactggggccgctggccccacatcagtgtccgtcatcccggcgtccagcccctggtgtgctgaagctcaggcccttccttgtgccctagtctcctgcactgagcttggtgagcccattcggatactgctggatgcatgggtcgggagggaggtgccctgggttgggtcgatgatgagaaccttatattgtcctgaagagaggtgatgacttaaaaatcattctcaaaaggattatgctgaggcccggcctaggttagaattttggaagaggatgctgggatcctgaggtccccaggagagcccctttatttttggctggagacctggaggccctgaagggtatctggggggcccagccctgtccctatactcctctgtgaggcagcccaggctctctgtgtgggcttggtgttggcactctggttttctgggggcggttcaggttgctggttccttctgaaccattgttctccccttgattctttcagtgagctcttctgctcaggcgggtcccctggcattgaccagcataggtaagtggatcctgctgggttcatgggccatgagccagaccacatggagtgactgagggtcagccttcaggacaggaagatttcctcggggagccaacctgaatccccaccgtggagggatgtgttagtcctccaggagggtggcttcctagggaatctgaccccaggaggacacctttgctgaagccctgcaggggtgatggtggtccaggctggaaagttttccttcagtctcatgtggggcagctccctgctgacattccgtgcctgcgtctgaccttgaggactgcccgggccacatgagtggggcaacaagggctccagggtgccacggggattatgagtggggctctccatccccggagagaggtgggcaatcaggcccatgtggtcaggcttgtagggttcggtggtggggagagccaaggtttccctgggtccattaccgtcactgtttcacctggatcccatcacatgggtccatggagggagagcatgtgtgaacaggcaccccttgctccctttcccacatgagcccctggttggacatatgtcctcctcctgtccccccagatggtggccctggaggaggacttgtattggggccactggccccacgtcagtgttcgtcagcctggcatccagcccctggtgtgctgaagctcaggcccttcctgttgccctagtctcctgcactgagcttggtgagtccattcggggactgctggatgcatgggcgggtagggaggtgccctgggttgggtcgatgatgagaaccttatattgtcctgaagagaggtgatgacttaaaaatcattctcaaaaggattatgctgaggcccggcctaggttagaattttggaagaggattctcggatcctgagatccccagccaggccactgaatttttggctggagtccttgaggccctgaagggtgtctgagggctgctgaaacctgtctctgtgcttgtccgtgaggcaacccacattcgctgtgtggacttggtgttggccttcccgttctccggggatgggacatactgtcagcttgtccctgagtacccgttctcccctgtgtctttcagtgagctcttctgcccaggtggtccccctggtcttgatgggcataggtgagtacatcctgctggtgtcctggtccatgagccagggtgcgtacggttgccggaggtcacctttaggccatgtgggtttcctcagggagctgacctgaatccccactgggagggatggtttctccaggaggatgtcttccatagaaactggttccaagaaggatgccttccctgaagctgtgcaggggtgacgatggtccaggcaggaaagggtccattcaacccaatgcagtacagcttctagctgagactcggtgcacgggtgagggcttgaggactcccaggcaacatgaggggttccacaggatctccggaggccacaggtgttaagcaggaactctccatccttgaggcttgctgggggtggcgggatggcagcagggtgtgggtgatcgagtttatggattcaggccaagattcaagggtcaggccccacccattgccctgatgggaccaaccctgttgtgcaatgggctgtcccactggtgcctccttgagcctggtgtgggcagcgtggcttcaatggggttgggtcaaggctgagctctggcccgcacctggtggttggccctgggaattgtggactttgggccaagggtgcagcccgcctgcctctggaggtcatcctacccagaggtggagtggccggaagggtggcaggcatcctgtggtgaaaggcccactgttgagggtcctgtcccaccaatttgctgttcaggctgcttctaagccatgggttcacctccagagaggcgggatcttgagcccagggaaaggagagggcatcatgtgtcccctgattggctgagaatggggagagtgtttgggtggtatgtggtgcatgaatggggacccaccagagaaggcgcgtggccctgggccccaggatgcaatttccggcgtgcttatagagggcgtctgagtggacactctccatgcccaggcgcaggctatgtgactgccaggtctgcagcagtttgtggcatctgggctagcctccttgattgcttatgagctggttgatagccctggcctgtgccgaggggtcctggtgcggccctggggttgttcaggggaagccggcatgggcagtggaggtgctcttggggcctgcagtagggcacagcccaggtgttcttgcttgagcgcagtgaaccacctgccctttccttccagggttcggtagcagggagggccaagggtcccccacgtccatgacaggtcagcgttcctcctggatcccatcacatgggtacatgcagggagagcatgtgtgagccggcactccccactctcttccccgcattgcagggtgaggccctgtttggacccatcttccagctatctcaccagatggtgagcccagaggaagaccccgtggctctgggccagtgtgcgtcagccaggtccccagcccccagtgtgctgaagctcaggcccttcccaacgccctggtctcctgcactgaggtgtggtgagcacaaccaggtcccactggatgcatgcacagggagggatgtgccctaggttgggtcgatgatgagaaccttatattgtcctgaagagaggtgatgacttaaaaatcatgctcaataggattatgctgaggcccagtctaggttaaaattctggaagaggatgctgggatcccaagttcacaggcagggccactgtattttggactagagaactcgaggccctaaagggcatcttgagggggtccaacctgtctctgtgctcctccatgaggcagccctggctccctgtgtggccttggtgttggtgctctggttcccaggcgtggcatgtgggcacctccttccagagcccccattctcccctgtctctttcagtgagctcttccgcccatttgggccctctgacattgaccggcataggtgagtggatcctctttgggtcatgggtcatgagcgaggcaaagtggggtcaccatgcatctgccttcgggccatgagagtttcctaaaagtgtggacctgaattcccaccggggatgagttatccagaagggccacttccttgggaatcggacccaagaaggacgcctgtcctgaagcccagcaggggtggtggtggtccaggcaggaaagggttcattcagcaccaagcaggcagctcccatctgagacttggtgcatcggtcaggccttgaggactgcccaggacacatgaggtggccacaggggctccggggcactacagggggtctgcagagaggggtaggcgattgggtccgtgggggtcatgccgcggagtttcaaggacacccatggccctgaatgggactgaccctgttgagtaatgggccatcccaatggcacctccttgagtctggcggtgggtggtgctgtttccctggggctaggtcaaggctgagctctggctagtgcctgtggtgctgcaggctggccctagaactcatgggctttgggccatggggacagcttgtgtcatgcttggggtcaccccttcccggaggttgaatggcgggagtggtggcttcctaaggtgggaagtcctgctattgagcaacctgtcctgggaatttgccacccaggctgcttctgagacatgaggtcacctccagagtggcagcatcttgggtccaaggaaagaggagcataacatgtgtccccagattggttgagtgtggcctagctctgtgtttgggaggtgtgcagggtgtggatggggatgtatcaaagaaagtacatagccctggagccccaggctgtgatttccagtgtgtgcttggagaggggcaggagcaggctctcgactgccaggtctgcagcagcgcgtggcatccgggcaagccttctggttggccatgtgctggttggtggccctggcctgtgcccagtggtctcagggtagacctggcattgtgcagggcagccagcagggcagtggaggtgctctcaggacctgcagtagggcgcagcctggactttttgtgtgagaccagtgacccacctgcactgtccttccagggttgggtggctgggagggccaagggtggtccatgtttgtctccgtgacacatcagtgctctgcatggatcctatcacatggatatcccctacccaccttccccacatggaagcatgaggccttacttggacatgtgtcctcctgtcctagatggtgagctcggaggaagacttgcattgggcccaatggagctgggccagtttctgtcagccaggtccccagctcctgatgcactgaagctcaggcccttcctggagccctggtctcctgccctgagctgtggtgagcccatctgggtcccactggatgcatgcatgggagggggttgccctcggttgggtcgatgatgagaaccttatattttctgaagagaggtgatgacttaaaaatcatgctcaataggattacgctgaggcccaacctaggggagaattttggaagaggacgctgggatcctgaggtccgtggcagggccaccgtattttgggctggagacctcaaggccctgaagggcatcttggcagcccccagccctgtctctgcactcctccgtgaggcagcccaggctccctgtgtgggcttggtgtcagagtcccattccccaggggcggggcaggttggcggctcctccctgagtcccctttctcccttgtgtctttgagtgacttcttctgcccaggtgggccccctggcgttgaccagcataggtgagtggaacctgctggtgtcatgggccatgagccaggccatgtggggactccaggtcagccttcaggacaggagagtttcctcagggagcaggggaatccccactggggagggatgggttctcccagagggccactttcttttgaatcggacccatggaggacatcttctctgaagacctgcagaagtgatgatgtttcaagcagcaaagggttttttcagcccctgcagggcaggcagctcccagatgagattctgtgcatgggtcaggcctcaagaactgcccaggccacatgcggggtgccacacaggctgtgaggcaccacaggggtcatgcaggagctctcggtcttcaggcttgttgtgggggtggtgacagagtgatcaggcccacagggtcaggctgcagtgtttcaaggatcgcgcccacctgtggccctgaatgggaccgatgctgttgagcaatgagccatcccagtggctccacattgagcctggtgggggcgccactgtttcagtggggatgggtgaagcttgagctctggccagcacctgatggtgttgcaggctggcccagggacttgtgggctttgggccaggtcggcagcctgagttctgctgggggtcacatctttcctgagtttcagtgtcaggatgcatggcctccttaggtgggaaggcctgctgtggagggtcctgtcctgggaatttgctgcccaggctgcttctgagccatggcgtcacctccaggtggtggggtcttgggcccaaggagcagagaagacaccatgtggcccctgattggtcgagtatggcctggctctgagtttgggaggtgtgcagggcatggatggggacccaccagagaaagcacgggccaatgaagccccaggctgcgatttctgatgcgcacttggagagggcgcctggttgggcgcccaccatgcccaagggtaggctatgcaactaccaggtctgcggcagtgcctggcatcaggaccagcctcctttgttggctgtgagctggttggtggccctagcccgtgtccagtgatctctgtgcagccctggggtcatgtggagcagccagtgtaggcaggggaggttatcttgaggtgtgcagtaggcacatcccaggcatcttgcccgagcccagtgacctccctgccctgtccttccagggttcggtggcggggaggcccaagggtaccctgtgtccatgacaggtcagtatttcatgtgaatcccatcacatgggccctcctcctgtccccccagatggtgagcctggaggaagacttgcactgggccttctgtgcgccggccagaggccatcaaccaggtgcccaggtccttttgtactgaagcttgggcccttcctggcaccctggtctccttcatggagctggtgagcccatccgggttcttctggatgtatgcatggggagcggggtgcactgggttgggtcaatgatgagaaccttatattgtcctgaagagaggtgatgacttaaaaatcatgctcaataggattacgctgagtcccagcctaggtgacaattttggaagtggacactgagaatcacaaggaccctggcagggccactgtattttgggctggagacctggaggccctcaagggcctctcacagggacccaaccctgtctctgcactcctccgtgaagcacagcaggctccctgtgcggacttggtgttggcactgtttcccaggggcagggcacattggtggcttttccctgagtccccattcttgccttgtgtctttcagtgagctcttcagctcagaagggccccttcaccttgattagcctaggtgagtgcatcctgctggtgtcatgggccatgagtcaggccgcgtggggtcaccaagggtcagtctttaggacaggagggtttcctcagggagccaacctgtatttccaccggggagggatggtttctacaggagggcggcttccttgggaactggacccaagatgttgccttcctgaagccctgcagggatgacagtggtccaggcagcaaagacttccttcagccccgtgcaggagagctcccagctgagactcagtgcatgtgtcaggcctcgaggattgccgaggccacatgatgggggccacagggcctccggggcaccacgggggtcatgcaagagttctctttccctggagaggggtgcttgaatgagcctatgggattgggccgtggagattcaaggattgtacccaactatgtcccaaaatgggactgaccctgttgagctatggaccatcccatggtgcctctttgagcttggtggggggcagtgctgtttccctgggtttgggtcaaggctgagctctggccagtgcctggtgaggctgtgggccggacctgggactcgtgagctttaggccaagtcgacagcttgtgtcctgctggaggtcaccccttccccatgggtgagtggctggaagagtggaatgtgcggtgggaaggcccgctgttgatggtcctatcctgagaatttgctgcccagactctctgtgccatggggtcatgtccagggtggcagggtcttgggcctagggaaaggaaaggacaccatgtggcccaggattggccaagggtggcccggatctgtgtttgggacatgtgtggggcatggatggggacccaccagagaaagcacatggccctggtactccaggctgtgatttcaggtgtggcttggagagagtgcctgggcaggtgcccaccatgcccagcggcaggctatgcaacttccatgtccacggtggcacatggcttctgggccaggctcctcggttggccatgagcatgtcggtgaccctgtcccatgctcagtggtcctggggcagccctgggatcgtgtgaaggagaagtcagagggggcactggatgtgccctcaaggtctgcaatagcatgaagaccaggggtcttgcccaagcccagtgatccacctgccctgttttttcagggtttggtgatggggagggccgagagtgccctgtatccgtgacaggtcagtgttccacatggatcccatcacatgggtatcccttaccccattccccacctggaggtatgaggccctgtttggacacatgtcctcctcctgtccatccagatggtgagcctgaaggaagacttgcatcaggaccaatggaccctggcaagtgtccgtcagccaggtgctcagcccctggtgcactgaagctcaggcccttcctggccccttgatctcctgcactgagctgtggtgagcacatccgggtcccgctggatgtatgtgtgggcagggggggtgccctgggttgggtcaatgatgagaaccctatattgtgttgaagagaggtgatgacttaaaattaccatgctcaatgattacgctgaggcccaacctaggtgagaaatttggaggaggatgctgggatcccgagatttccggcagggccactgtattttgggctggagccctggaggccctgaaaggtatctggaggaggcccaactctgtctctgcactcctctgtgaggcagcccaggctccctgtgcgggcttggtgttggcctttctgttccccagtgaagtggcacgtgggtggctcctccctgagccccagttctccccttgtgtctttcagtgagctcttctgccaagggggggcctcctggcattgaccaacataggtgagtggatcctgctggcatcatgggcatgggttcactgagggtcagacttcaggccatgagggtttcctcagggagctgaccccaattcccatcagggagggatggtttctccaggagggcggcttccttgggaatcttacctgaggaggatgccttccctgaagatctgcaggaatgacaatggtccagatagcaaagggttccttcagccccatgcaggacatcgcccagttgagacttggtgtgcaggtcaggccttgaggactgcccaggccacatgaggcgggccagaagggctccagggtgccacgggtttcacttgggagctctcggtccccgggcatgttcctggggggggtggtgatggggtcgggccatggaggttcaaggatctcatcccacctgtgaccctgaatggggctgatcctgttgaacaatgggccatcccagtggcacgtattgagcctggtcaggggtggtgcagtttcaatggggttgggtcaaggttgagatctggccagctcctggtggtgcttcaggccagtcctgtgacccatgggctttgggctaagggggcatcccatgtcccatgggggtcacctgttctccatgtttgagtggcagtaacagtggcattctgaggtgggaaggtccactgtctagggtgttgtaccgggaatttgccaccccgccttcttagccaaggggtcacctccagggtctcgggatcttgggcccaaggaaacgagaagataccatgtggcccttgattggccgagtgtggcccagctctgtgtttgagaggtgtgggatatgaatggggtcccaccagtgaaagctcatggcccaggtccccaggctgtgatttttagtgcacacttggggagggctcctcagtgggtgcccaccacgcccagaggcaggctatgtgactgccaggtctgcaacggtgcatagcttccaggctaccctccttggttggctgggagcgggttggtggccatggcccatgcccagtggtcacagggcaaccctgggatcatgcaggggagctggtgagggcagtggaggtgctctccggaacggaagtatggctcagcccaggtatctggcccaaacccaatgaccagcctgccttgtttctcagggtttggtgatggggagggccaagggtgccctgcatctgtgatgggtctgttccacatagaacccattacaggggatcccttctccacatggagatgtgaggccctatttggacacatatcctcctcctgtccccccagatggtgagcctggaggaagatttgcattgggcccaatggccctgggccagtgtctgtcagccagtttcccagcccctggtgcactgaagctcaggcctttcctggcatcctggcctcctgcactgagctgtggtgagtatatcatggtcctgctggatgcatgtgcagggagggaaggtgccttgggttcagtcagtgtcgagaaccttatattgttctgaagagaggtggtgacttaaaaatcatgctcaataggattacgctgaggcccagcctagttgagaattttggaaggggacagtaggatcccgaagtccctggcagggcccttatattttaggctggagacttcaaggccctgaagggcacctggaggtggctcaacctgtctctgtgctcctccatgaggcagtccaggctccctctgtgggctttctgttggcactctggttccctgggccagggaacgttggtggctcctccctgagcccctgttctccccttgtgtctttcagtgagtgcttctgcccaggcaggccacttggcattgaccggtataggtgagtggatcctgctggggtcatgggtcatgagcaaggccacatgggtcgccgagggtcagccttacagccataagggttttctcagggagcccacctgaattcccaccggggaggcataggttctccaggagggcggcgttttggggaatcagacccaagggggacaccttccctgaagccctgcaagggtgatggtggtccaggcaggaaagggtttttttcagccccacacaaggcagctcctagctgagaatcggtgcttgggtcagacctcaaggattgcccaggccacatgagggcgactgagagcctccggggcaccacaggggtcacgtgggagctctcggtgcctggcgtgtgatgcacaaatggacctcttgggtttgttcatggagattcaaggatcgcaccccaccatgcccctgaatgggatggtccctcttgagaaatgggccatcccagtgggtctcctggagcctggttggggggtggcattgtttccctggggttgggtgaaggcagagttctggccggcacctggtggtgctgcaggtcagcactgggacccgtgggcgttgggccaaaggagagcccgaatcccactgggggtcaccctttttccaacatcaaatgtctggaagggtggcctcctgaggtgggaagtcctgctgttgagggacatgtcctgggaatttgtcactcaagcttcttctgagctttggtgtcacatccagggtggcagggtcttgggtccaagcaaagcagagggcaccatgtgtctcctgattggccaagtgttgctggctctgtgtgggaggtgtgcagagtatggatggggacccacgagagaaagcacgtagcactgcaactccaggctatgatttctgttgcatgcttggagagggtgcctggacaagcgtgccatgcccaggggcaggctgggcaactaccaggtcttcagcggagtgtgacatctgggccagcctccttggttggccgtgacctggttggtggcccggctctgtgcctactggccccagggcagccctgggatcatgcaggggagccattttgaacagtggaggtgctcttggggcctgtagtatggcacagcctaggtgttttgctcaagcccagtgacccacctgccctgtcctctccgggatcggtggaagggagggccaaaggtgctccgtgtacttgatgagtcagtatttcacattgaactcatcacatgtgtccatggagggtgagcaagggtgagatggcactgctgacccccttccccacgtagaggtcagtttggacacttgtcttcctcctgtctccccacatggcgaccccagaggaaaacttgtgttgggcccgctgtcactgggccaatgtcagtcagccaggtgcccagcccctggtgcgctgaagcttgggcccttctggcacccttggctcctgcactgagctgtggtgagcccatctgggtcctgctggatgcatgtgcagggagggggatacctgggttaggtcgattatgagaaccttatattgtcctgaagagaggtgatgacttaaaaatcatgcccaataggattacgctgaggcccagcctaggtgagagttttggaagaggatgctgggattctgaggtccctgacagagtaaatgtattttgggctggagaccttgaggccctgaagggcatctggtgggcccacccctatctctgtgctcctccgtgaggcagcccaggctccctgtgctggcttggtatctgggctccggatccctggttatggggcatgttggagtctccttcctgagtccccgttctcccctgtgtcttttggtgagctcttctgcccaagtggtccccgcggccttgagcggcataggtgagtgcatcctgtttcggtcatgggccatgagtgatgcagcatggggtcctcgacattcagccttcgggccactagggttttctcaggctgccaacctactccccaccagggagcaatggtttctccaggagggccacttccttgggaatctgacccaaggagcacatctttcctgaagccctgcaggggtgacagtggtccaggcagcaaagggttccttcagcccaatgtagggcagctcccagctgaaactcggtggacagctcaggcctggagtactgtccagggcacatggggaggaggggcacagggaatgtggcgcaccctagaggttacactggagttctctctccctggagaggggtgagtgattgggcccacacagtcaggctgtgaagattcaaggaatgctccacacccatggccttgaatgggaccaaccctgttgagcagtgggccatcttatggtacctccttgagtctggtgagggatggcactgtttcagtgaggttgggtcaagactgagctctggctggcacctggtgctgctgcaggccctgggacttgggggccttcagtcaagggggcagctctgatcctgctggggacaaaccttccttgaggtccagtggtgaagagtgtcctcctaaggtgggaaggcctgctgtcaagggaatttgccacccaggctgcttctgagccttggggtcacctcagggtggcagggtcttgtgcccaaggaaaggagaagacaccatctggttcctgattggctgagtgtagccgggctctgtgtgtcggagttgggcagggcatggatggggacccaccagagaaagtgtggcccaggggccccccaggcttggagagggcaccagggccggtgcccacaatgctcaggtgctggctctgggagggccaggtctgcagcggcttttggcatctggccaaccaccttggttggccaggagctggttgatggccctgtcctgtgtcctgtgccctgtgccctgtgtccctggtatggccctggggtcatgtgtgggagctggcctgagcagtggaggtgctcttggggcctgcagtagggtacagcctaggcatcttgcctgagccagcactgtcttttgctttccctgtctttccagggtttggtggcagagagggcaaaggatgccccacatctgtgttgggtcagtgttcagcatggatcccatcacatgggtatcccttagccccttccctgcattgatatgtgaggccctggttggacacatgtcctccttctgtccctctagatggtgagcctggaggaagatgtgtgggacccgcaggccgagggccagtgtctctcagccaggtgctcagcccccagtgggctgaaactctgacccttcctggcaccctgctctcctgcactgtgctgtggtgagcacatccgggttccactggatgtttgtgcagggagggggttccctgggttgggtcaatgatgagaacagggccacatgaggggggccagaggggctacagagtgccatggggtaacaagggagctcttggtccctggagtgtggtgtgtgaatgggcccatggggttgggtagcagagattcaaggatcctgctctacccatggccctgaatgtgaccatcgctgttgagcaatgggccatcccagtgggtctcctggggcctggttggggggcggagttgtttccctgggattgggtcaaggttgactctggctggcacctggtggtgttgcaggtcagccctgggactcatgggctttgggccagggacagcccgcatcccgctgggggtcacccaagtgttgtgaagggtggcctcctgagttgggaaggcccgctattgaggcatctgtactgggaatttgttgctcacgctgcatgggagccatggtatcacctccagggtggcagtcttgggaccaagcaaaggagggggcagcatgagactccggattggctatgtgtgactggctatgtgtcttggaggtatgtggagtatggatggggacccaccagagaaaacaagtgaccctgaggccccatgctgtcttttctggtgcctgcatggagagggtgcctgtacgcatgcccaccatgcccaggggcaggctgtgccactgtcaggtctgcagtagcatgtggcatctgggccagcctccttggttggctgtgacctggctggtggcccggacctctgcccagtggtcccagggcagccctagggtcatgcggaggagcagttttggccagtggaggtgcccttgggtcctgcagtcgtgcataccctgggcgttttgcctgagcccagtgacctacctgccttgtccttcccgggttcggtggcagggagagacacttgcataggtgatgagtcagtatttcgcatggatctcatcacatgtgtccatggagggggagcaagcgtgagctggcacccaacacccctttctccacatggagggtggtcacatgttctccacctgttcccctagatggtgagacttctgttgggcttgcttttcctgggccagtgtccatcagccaggtgctcagccatcggtgcgctgaagctccagcccttcctggtgccctgatctcctgcactgagctgtgatgagtacatccgggttccactggatgcatgtgcgggaaggggggtgccctgggttgggtcagtgatgagaaccttatattgtcctgaagagaggtgatgacttaaaaatcatgctcaataggattacgctgaggcccagcctgggggagaattttggaaaaggatgctgggatcccagggtccccaacagggccactgtattttggacctgggacctggtggctctgaagggcatctggaggtggcccaacctgtctctgcactctagtttccctgtgtgggcttggtgtcggtgctccagttccctgggggcagggaatgttgatggctccttcctgagtccctgttctcgcctttgtctttcagtgagctctttcacctaggagggccccttggcattgactggcataggtgagtggatcctgctggtgttatggatcatgagccagaccatatggggtccccaaggatcagcctttgggccacaagggttttctcagggagctgacctgaatttccacctgagacggaaaggttctcctggaaggcaggctttttggaatcggacccaaggaggacaccttctctgaagccctgaaggggtgatggtggtccaggcaggaaagggttccttctgcctaacataggacagtgcctagttgagactcggtgctgcagtaaggccttgaggactgcccaggctacatgaggggggccagaggggctacggggcaccacagggttaatggtgacctcttggtccccagagtgtggtgcgtgaatggacccatggtgttgggttgtggagattcaaggatagtgccccacctatggccctgaatgcaactgtccctgtttagcaatgggccatctcagtgggtctcctggagcctggtggggggcagagttgtttccttgtgactgggttaaggtagagctctggccagcacctggtggtgtggcaggtcagccctgggactcttgggcttttgaccaaggggcagcctgtatcccgctgggggtcatcctatctctgaggtcaagtgtcgtgaagggtggcctcctgaagtgggaaggctcgctgtcgagggacctgtcctaggattttattgctcaggctgcttctgagccatggtgtcacctctagggtgatggggtcttgggaacaagcaaaggggagggcaccatgttgctccggattgggcaagtgtggcccagctctgtgtgtgtgaggtgtgcagaacatggatggtgacccaccagagaaagcacatggccctgtagctctaggttgtgatttccgtgcacacttggagacagcatcttgatgtgtgcccaacacgcccaggggcagactatgcgatagccaggtctgcagcggtgtgtggcatctgggccagccacctttgttggccatgagctggctcatggccctgacccatgcccactggtgtcagggggccctggcgtcgtgtgaggagccatctgtggcagtggaggtagactcagggcctgcagtatggcgcaacccatgcattttgcctgagctcagtgacctacctgccctgttcctccagggtttggtggcagggggagccaagggtgccccacattggtgacgagttagtacttcacatggatctcataacatgtgtccgtggagggcaagcaaatgtgagcccgcacccactactgccttccccacatggaggtcagtttggacacatatcatcctgctgtcccccaaatggtgagaccgaagaagacttgccctgggccctctgttcttgggccagtgtcagtcagccaggtgcccagctctaagtgcgctgaagcttgggcctttcctggaatgctcggctcccgcactgagccagggtgagcacatccgggtactgctggatgcatgtgtggggcaggggtgcgccctgggttgggctgatgatgagaaccttatattgtcctgaaaagaggtgatgacttaacaatcatgctcaataggattacattgaagcccaggataggtaagaattagggaagatgatgcagggatcccaagatctccagctagaccacacttattttgcactggagaacttgagaacttgaagggcatctcaggggagcccaaccctgtctctgagctccttcgtgaggcagcccaggctccccttgcgggcttggtgtctgtgctccgcttctccagggacagggcaatttggtgttccttcctgaatgctccttcttccctgtgtccttcagtgagctcttctgcccaggtggggtccctagctttgagcggcataggtcagtgcatcctgttggggtcatgggccatgagtgaggcagcatggggttggtgagagtcagccttcaggccactagggttttctcagggagccaaccgattactcaccgggagggatggttcctccaggagggtcacttcttgggaatctgaccctaggagcacacatttcctgaagctcggcagggagatggtggtccaggcaggaaagggttccttcaacctaacgtagggcagttcccagctgaaactaggtgggcaggtgaggcttgaggactgcccagtccacatgaaaggggccacaggggctttggggtgccctgggagtcatgctggagttcttcccccaagagggatgggtgatagggtccacgtggtcaggctgcggagattcaaggaatgccacccacccatggccctgaatgggaccaaccctgttgagcaatgggccattttcggcatgtttcaatgcagttgggtcaaggctgagcgctggctggtgcctggtggtgctgcaggccggccctggggctcatgggctttatgccaagggggcaactcccatcccactggggtaatcccttccttgaggtccagtggtgggaagggtggcctcctgaggtgggcagacctgctgtcaagggacctgtcctgggaatttgccacctaggctgcttctgagccatggggtcacctccaggatggcggggtcttcagcccaaggaaaggagaggacaccatgtggtttctgattggctgagtgtggcctggctgtatgtgtgggaggtgtgcagggcatggatggggactcatcagagaaagaatgtggccctggaggcccccaggctttgatttctggtgtgtgcttggagtgggcaccagggtggccctaccatgcccaggggcaaggtctttgcctgccaggtctggagtggctcctggcatcaggaccaccctccttggctcaaccaaccttcagcaggttggctgccttggcccgtgccccatggtcccggtacgaccctggggttgttcgggggatctggcccaggcagtggaggtgctcttggggcccacagtagggcacagcccaggcctcttgccccagcccagtaacccacttgccctgtctttccagggttttgtggcagggaaggccaagggtgctctgcgtccatgttgggtcagtgttctgtaaggattccattacaagggtatcccctacccaccttccctacattgatacatgaggccctggttggacacatgtcctcctcctgtccctacagatggtgaccctgaagaaagacttcagttgggcctgaaggccccgggccagtgtccatcatccaggtgctcagctcccagtgcactgaagctcagacccttcctggttccctggtctcttgcactgagctctggtgagcacaactgggtgctgctggatgcatgcatggggaggggggtgccctgggttggggtggtgatgagaaccttgtattcttctgaagagaggtgatgacttaaaaaccatgctcaataggattacacttaggccgaacctaggtgagaatgttggaagaggacgttgggatcctattatccctggcagagccactgtattttgggctggagacctggaggccctgaaagggcatctggagggggcccaaccctgtctctgtgctcctgcatgagggtccccaggctccctgtccagccttggtgttggtgctctggtaccctggggatggaacaagttggtggctctttcctgagcccccattctccctttgtgtctttcagtgagcttttctgcccaggtgggcaccctggcattgattagcctccttggttgtccatgacctggctggtggcccgatcctgtgcccagtggtcccagggcagccctggcatcatacggggcagctgttttgggcagtggaggtgctctccaggcctgaagtatggcacagcctgggcattttacctgagaccagtgacctgcctgccttgtccttcctgggttcggtggcagggagggccaagggtgccctgcgtcagtgatgagtcagtattttgtgtggatctcatcacctgtccatggagggaaagcaagcatgagctggcacccaacacccccttctccgcatggagggtggtcacatgtcctccacctgtgcccccagatggtgagacttgaggaagacttccactgggcctgcttttcctgagccagtgcccatcacccaggtgctcagtccctggtgcactgaagctccagcccttcctggcgccctggtctcctgaactgagctgtggtgagcacatccgggtttcactggatgtttgtgcggggagggggttctctgggttgggtcaatgatgagaaccttatattgtcctgaagagaggtgatgacttaaaaatcatgctcaataggattacgctgaggcccagcctaggggagaattttggcagaggatgccgggatcccaagatttccgggagggccacttgtattttgggttggagacctggaggccctgaagggcgtctggaggtggcccagtgctttctttgcgctcctctgtgaggcagcccaggctccctttgtgggcttggtgtttgcgctcctcttctccagtgatggggcacgttgggggctcctccctgagtcctggttctccccttgtgtctttcagtgagctcttctgccaagcggttcccctggcattgaccaacataggtgagtggatcctgctggcatcatgggctatgagctaggccgcatgggtttgctgagggtcagacttcaggccccaagggtttcctcagggaaccgacctcaattcccaccatgtgactgccaggtttgcaacagtgcatagcttccgagctaccctccttggttggccgggagctggttggaggctgtggcccatgcctagtagtcacagggcaggcagactgccctgttttttcagggtgtggtgacggggagggccaagggtgccctgcatccatgatgggtcagggatttcaggagggatggtttctccaggagggcagcttccttgggaatcttacccaaggaggatgtcttccctgaagccctgcaggggtgacgatggtccagatagcaaaggtttccttcagccccatgcaggacatcacctagttgagatatggtatgtgggtcaggccttgaagactgcccaggccacctgaggggcccagaagggctctggggtgccacaggtgttatgtaggagctctcagtccccagacttgtttcgtggggttggtgacggggttgggttgtggaggttcaaggatttcatcccacctgtgaccctgaatggggccaaccctgttgagcaacaggccatcccaatggcacgtattgagcctggtggggtgtggtgcagtttcaatagggttgagtcaaggctgagatccatctggtgcttggtgctgctgctggccagccctgggacccgtgggctttgggctaagggggtagcccacatcctattgagggtaacccattctccgagtttgagtaacggttaacagtggcattctgaggtgggaaggcccactgtctagggtcctatactgggaatttgccaccccgggttcttctgagacaagtggtcacctccagggtcatggggtcttgggcccaaggaaaggagaagataccatgtggcccttgattggctacatgtggcccaggtctgtttgggaggtgtgtgggatgtggatggggtcccaccaatgaaagcacatggcctgggtccccaggctgtgattttcagtgcacacttggagagggtgcctcggcgggtacctaccacatccatgggcaggctatgtgactaccaggtttgcaacagtgcatagcttccaagctaccctccttggttggtcgggagctggttggaggctgtggcccatgcctagtggtcacagggcaggcagacggccctgttttttcagggtatggtgatggggagggccaagcatccatgatgagtcggtgttccacatggaacccattacgtgggtatctcttaccccttcctcatatggagatgtgaggccctatttggacacatatcctcctcctgtacccccagatggtgagcctggaggaagacttgcgttgggcccaaaggccctagaccagtgtctgtcagccaggttcccaacccccgatgcgctgaagctcaagcctttcctggcaccctggtctcctgcactgagctgtggtgagtatatcctggtcctgctggatgcatgtgcagggaggtgggtgccttgggttgggtcaatgatgagaaccttatattgttctgaagagaggtgattatttaaaaatcatgctcaataggattacgctgaggcccagcctagttgagaattttggaggaggacactgggataccgaagtcccccgcagggactctgtattttgggctggagaccttgaagccctgaagggcatctggaggtggcccaacctgtctctgtgctcctccctgaggcagcccaggctccttttgtgggcttggtgttggggctccagttccccaggtcagggaatattggtagctctttactgagcccctgttctccctttgtgtctttcagtgagctcttctgcccaggcgggccccctggaattgatgggtataggtgagtgcatcctgctggggtcatgggccatgagccaggccacgtggggtcaccaggggtcccccttctggccatgagggttttctcagggaaccgacctgaattaccactggggaggcataggttctccaggagggcggctattttgggaatcggatccaagggggacaccttccctgaagccctgtgggtgtgatggtggtccaggcaggaaagggtcccttccaccccacacagggcagctcatagctgagagttgatgctcaggtcaggcctcaaggatttcccaggccacatgagggggacagaggagctctgggtgccacgggggtcacataggagctctcagtattcggtatgtcatgcacaaatgggcctcttgggttggattgtggagattaaaggatcgcgcaccaccgtggccctgaataggaccatccttattgagcaatgggccatcccagtgagtctcctggagcctggtggaggggagggggtggcattgtttccctggggatgggtcaaggcagagctctggccagcaccttgtggcttctgcaggtcagccttgggactcatgggcattgggccaagggggagcccgcgtcccactgggggtcaccctttttccgacgtcaagtatctggaagagtggcctcctgatgtgggaaggcctgctgtcgagggacgtgtcccaggagtttgtcgctcaagtttcttctgagctttggggtcacatccagggtggcggggtcttgggcccaagcaaagcagagggcaccaagtgtctcctgattggccatgtggctgtctctgtgtgtgggaggtttacagagtatggatggggacccaccagaggaagcacgtggccctgcagcaccaggctgtgatttctgttgcacacttggagaggttgcctggacacgtgcccttcatgcccagtgacagactatgcaactaccaggtctgcagcagggtgtgacatctgggctagcctccttggttggccgccacctggctggtggcccggatctgtgcccaacagtcccagtgcagcactggggtcatgcaggggagctgttttggacagtggaggtgctctcggggcctgcaatatgatgcagcctgggcgttttgctcaagcccagtgacccacctgccctttcctttctgggttcagtggaagggagggccaacagtgtagtgcgtacttaacaagtcagtatttcacattgaactcatcacatgtgtccatggagggcaagcaaaggtgagctggcacccccgacccccttccccaaacggaggtcagtttgggcacacgtctccctcctgtctccccagatggtgaccccggaggaaaatttgtgttgggcccactatccctgggccaatgtcagtcagctaggcacccagcccccagtgcgctgaagctcaggaccttcctagtgccctcggctcctgcactgagctctggtgagtccatctgggtcctgctggatgcatgagctgggggcgggagggtgccctgggttgggtcagtgatgagaaccttatattgtcctgaagaaaggtgatgacttaaaaatcatgctcaataggattacactgaggcccagcctaggtgataattttggaagaggatgctggaattctgaggtccctgacagagcagctgtattttgggctggagaccttgaggccctgaagggcatcttgtgggcccacccctatctctgtgctcctctgtgaggcagcccaggctccctgtgctggcttggtatccgggctctgggtccctggtgacggggcatgttggaggctccttcctgagcccctgttctcccttgtcattcagtgaactcttctgcacaagtgacccccccagccttgagcggcataggtgagtgcatcctgttttggtcatgggccatgagtgatgcagcatggggtccctgacattctgcctttgggccaccagggttttctcaggctgccaacctactccccaccagggagcgatggtttctcaggagggctgcttccttgggaatctgacccaaggagcaagtctctcctgaggccctgcaggggtgacagtggtccaggctggaaagggttccttcagcccaatgtagggcagctcccagctgaaactcagtgggcaggtcaggcctcgagtattgcccaaggcacatgggggagtgaggcacgggggatacagtgcgccgtggaggtcacactggagttctcccctccctggagagggatgagtgactgggtccacacggtcaggctgtggagattcaaggcatgctccacacccatggccttgaatgggaccaaccctgttgagcaataggccatcttagtggcacctccttgagcctggtgggggacggcactgtttcaatgaggttgggtcaagagtgagctctggctggcacctggtggtgctgcaggccctgggacttgtggactttgggccaagggagcagctctgatcctgctggggacaagccttccttgaggtccagtggtgaaaagagtgtcctcctgagatggcaaggcctgctgtcgagggacctgttctaggaatttgctgcccaggctgcttctgagccttggggtcacctcagggggtggcagggtcttgagcccaagaaaggaaaagacacgatgtggttcctgattggctgagtgtgacccggctctgtgtgtcagagttgggcaggacatggattgagacccaccatagaaagcatgtgatcctggaagcccctaggcttggagagggcaccagggccagcgcccaccacgcctaggtacgatctctcggacttccaggtctgcagcggctcttggcgtctgggccagcatgcttggttggccaggggctggttggtggccctgtcttgtgccctgtgtccccggtatggccctggggttgtgtgtggtagcaggcctgggcagtggaggtgctcttggggcctgcagtagggtgctgcccaggcatcttgcccgagccaggtgacacgcttgccctgtctttccagggtttggcggcagggagggccaagggtgcccagtgtccgtgagggatcagtattcagcatggatcccatcacatgagtatcccttagccccttctctacattgatgtgtgaggccctggttagacacatttcctcctcctgtccctccagatggtaagcctggaggaagacttgtgttgggcccacaggccccaggccaatgtctgtcagccaggtgctcagcctctggtgcgctgaaactctgacccttcctggcacactggtctcctgcactgagctggggtaagcacatctgggttgcactggatgtttgtgtggggagggggttccctaggttgggtcaatgatgagaacctgatattgccctgaagagagatgatgacttaaaaatcatgttcaataggattacgctgaggcctagcctaggtgagaattttggaagcagatgctgggatcccaaggtcccttgcagggccatcgtatgttgaactggagacctggaggctctgaagggcatctggaggtcgtgcaacctgtctctgcactcctctgtgaggcagcccaggctccctgtgtggggttggtgttggtgctatggttccctgggggtggggaacattggtggctccctccagagcccctgttctcaccttgtgcctttcagattttccacctaggaggtccacctggcattgactgacataggtgagtggatcctgctggggtcatgggtcatgagccagaccatggggggatcccagagattcagtctttgggcaacagttttctcagggagcagacctgaatttccacctgggagggaagggttctcctggagggcgacttttttgggaatcggacccaaggaggatgccttccctgaagccctgccggggtgatggtggtccaggcaggaaagttccttctgctcaatgcagggcagcacccagctgagactcggtgctccggtaaggcctccagaactgcccaggtcacataaggggggccagaggggctacgaggtgccacggggtaacaggggagctcttggtccccagagtgtggtgcatgaatgggcccattccgttgggtctcagagatttaaggattgcaccacaccatggccctgaatgcaaccgtccctgttgatcaatgggccatcccatgggtctcctggagcctggtgggaggcagtgttgttttcctggggttgggtcaaggcggagctctggctggtacctggtggtgctgcaggtcagccctgggacttgtgggctttgggccaaggggcatcccgcatcccgctgggggccaccctttctccatggtcgagtgtcgtgaagggtggcctcctgagttgggaaggcttgctattgaggcacttgtactgtgaatttgttgctcaggctgcttgggagccatggtgtcacctccagggtggcagggtcttgggaccaagcaaaggagtgggcaccatgtcactgcagataggccatgtgtgactggctctgtgtctgggagatatgtggagtatgaatggggacccaccattcatagagaaagcaagtggccctgcagcccgaggctatgatttccggtgcacacttggagagggtgcctttacacccaagtaccaggctcagaggtaggctatgccactatcaggtctgcagtggcacgtggcatctgggccagcctccttggttggccatgacctggctggtggcctgggcctgtgcccagtgttcccagggtagttctagggtgctgcgggggagctgttttggctagtggaggtgctcttgggtcctgcagtacagcacagcctgggcgttttgcctgagcccagtgacctgcctgccttgcccttcccaggatcggtggtagggaagaccaggggtgcccggcataggtgatgagtcagtatttcacatggatatcatcacatgtgtccatggagggggagcaaacatgagctggcacccgccacccccttctccacatgaagggtggtcacacgttctccatctgttcctccagatggtgagacttctgtcggcctcgcttttcctgggccagtgtccatcagccaggtgctcagccccagtgcactgaagctccagcccttcctggcgccctggactgagctgtggtgagcacatccgggttccactggatgcgtatgtgggaaggggggtgccctgggttgggtcaatgatgagaaccgtatattgtcctgaagagcggtgatgacttaaaaataatgctcaataggattacgctgaggcccaccctaagtgagaaatttggtaggggatgttgggatcccgagatttctggcagggccactgtagtttgggttggaggcctggaggccctgaagggcatctggaggtggctcaactctttctctgtgctcctccgtgtagcagcccaggctccctgtgcgggcttggtgttgttgcttctgttccttgggggcagggcatgtgggtggctcctccctgagccccagttctccccttgtgtctttcagtgagctcttctgccaagggggtcctcctggcattgaccaacataggtgagtggatcctgctgctgtcatggtccgtgagccaggctgcttggggttgccaagggtcagacttcgggccatgaaggtttcctcagggagctgaccctaattcccatgagggaggcatggtttctccaggaggtcagcttccttgggaatctgtgacttgacagggttgagtcgcagaggttcaaggatctcatcccacctgtaaccctgaatggggcccaccctgttgagcaactggccatcccagtggcacatattgagcctggtggggggtggtgcagtttcaatggggttgggtcaaggctgagatatggctggtgcctggtggtgctgcagtccagccctgggatccgtgggctttgggctgagggggaagcctgcatcctattgggggtaacctgttctccaaatttgagtgattggtgggaaggcctactgtctgaggtcctgtactgggaatttgccacctcagcttcttctgagccatagggtcacctccagggtcatggggtcttgggaccaaggaaaagagaagataccatgtggcccttgattggccgagtgtagcccagctctgtgtttgggaggtgtgtgggatgtggatagggtcccacctgtgaaagcatgtggcccgggtccccaggctgtgattttcagtgcacacttggagagggcaccttggtgggtgcccaccatgtccaggggcaggctatgtgactgccaggtttgcaatggtgtgtagcttccttctaccctccttggttggctgggagtgggttggttgctgtggcccatgcccagtggtcacagggcaggcagactgccttgttttttcagggtatgctgacagggagggccatgggtgccctgcattcatgatgagtcagtgttccacatggaacccattatgtgggtatctcttattccttctacatatggagatgtgaggccctatttggacacatgtcctcctcctgtacccccaggtggtgagcctggaggaagacttgcattgggcccaatggccctggaccagtgtctgtcagccaggttcccagcccccgatgcgctgaagctcaagcctttcctggcaccctggtctcctgcactgagctgtggtgagcatatcctggtcctgctggatgcatgcgtggggaggggttgtcctaggttgggtcaatgatgagaaccttataatgttctgaagagaggtgatgacttaaaaatcatgctcaataggattacgctgaggcccagcctaggtgataattttggaagaggacactgggtcccgaagccccctgcagggcccctgtgttttgggctggagaccttgaggccctgaaaggcatctggtggacccaactctatctctgccctcctcaatgaggcagcccagcctccctgtactggcttggtatccgggctccgggtctctggggacggggcacattggaggctccttcctgagcccccattctctcctgtgtctttcagtgagctcttcggcccaagtggtccccccggccttaagcggcataggtgagtgcatcctgttttgatcatgggccatgagtgttgcagcatggggtccccgacattcagccttcaggccactaggcttttctcaggctgcaaacctactctccaccagggagcgatggtttctcaggagggccacttccttcggaatctgacccatggagcacgcctttcctgaagccctgcagaggtgacagtggtccaggcaggaaggggttccttcagccccaatgtagggcagctcccagctgaaacttggtggacagatcaggcctggagtactgcccagggcatatggggaggagggacacagggaatgtggcgcaccctagaggtcacactggagttctgtctccctggagaggggtgagtgattgggcccacatggtcaggctgtggagattcaaggaatgctccacaccgatggccttgaatgggaccaaccctattgagcaatgggccatcttagtggcacctccttgaacctggtgggggatggcactctttcaatgaggttgggtcaaggctgagctctggctggcacccagtggtgctgcaggctggccctgggacttgtgggctttgggccaagggggaagcttccatcctgctgggaacaacccttccttgaggtccagtggcaggaaggggggcttcctgaggtggaaagccctactgttgagggacctgtcctgggaatttgctgtccaagctgcttctgagccatggggtcacctctagggtggtggggtcttgagcccaaggaaaggagaggacaccatgtggttcctgattggctgagtgtggcccggctctgtgtgtcggagttgtgcagggtatggattgggacccaccagtgaaagcacatggccctggaggtccgcaggcttggagagggcaccagatctggcacccaccatgcccaggtacaggctctgggactgccaggttgcagcagctcttggcatctgggccaccctccttggttggccaggagctagttggtggccctgtcccatgccccatgtccctggtatggccatggggtcatgcagaggaaccggccctggcatggaggtgctcttgcaacatgcagtagggtgcatcccaggcatcttgcctgagccaggtgacccgcttgccctgtctctccagggtttggtggcagggagggccaagggtgccccgtgtccatgttgggtcagtgttcagcatggatcccattgcatgagtatcacttagctccttcccagcattgatatgtgaggccctggttggacacttgtcctcctgtccctccagatggtgagcctggaggaagactcgtgttgggcccacaggccccaggccagtgtacgtcagccaggtgctcagcccatggtgtgctgaagctctgacccttcctgactccgtggtctcctgcactgagctgtgatgaccatatccaggtcctgctagatgcatgcgtggggaggcagggtgccctgggttgggtcaatgatgagaaccttgtattatcttgaagagaggtgatgacttaaaaatcatgctcaataggattacactgaggcccagactaggtgagaattttggaagaggatgctgggatcctgaggtccccaacagggccaccatattttgggctggagacctggaggtcctgaagggcatctgtagggggcccaaccctgtctctgcactcctccctgaggcagcccatgctccctattcgggatcggtattggtgctccacttccctgggggcaggacacgtgggtggctactccctgagcccccgttctccccttgtgtctttcagtgagctcttccaaacaggtgggctccctagcattgaccgacagaggtgagtggatccttctgggattatgggccatgagccaggccatgtggggtccccaaggttcagcctttggatcccgagagttgtctcagacagccaacctgatttgccacctgggagagatggcttctccaggagggtgggtttttgggaattggacccaaggaggacgccttccctgaagccctactgggatgatggtggtccaggcaggaaagggttccttcacccagctgagactcggtgttccagtaaggcctcaaggacctccctgccacatgaaggggcccagaggggctacgaggcaccacaggagtgacaggggagctcttggtccccagagtgtggtacatgaatgggcccatggggttgagtcgcagagattcaaggatgctgcaccacccatggccctgaatgtgactctcctcctggagcctggtggggggtggcgttttttccctggggttcggtcaaggtggaactctggtcagtacctggtggtaatgtaggtcagccctgggactcgtgggctttgggccaacggacagcccacatccctctgggggtcaccctttctctgaggtcgagtgtcgtgaagggtggcctcctgaggtgggaaagcccaatgtcgaaggacctgtactgagaatttgttgcttaggctgcttaggatccatgttgtcacctccagggtggtgggatcttgggaccaagcaaaggagagggcaccatgtggctgccagtggctatgtgtgactggctctgtgtctgagaggtgtgtggatggggacccaccagagaaagcaagtggccctgcagccccaagatgtgatttctggtgcaggcttggagagggtgcctatacctgctcccaccacgcccaggggcaggctttgccactgcaggtctgcagtgccacgtggcatctgggccagccaccttggttggccgtgacctggctggtagcccgtgcctgtgcccagtgttcccagggctgccctggggtcttgctggggagctgttttgggcagtggaaatgctcttggggcctgcagtatggcacagcctgggggcattttgcctgagcccagtgacctgcctgccttgtccttcctgggttcggtggcagggagggccaagggtgccctacattggtgacaagtcaatattttgcatggatctcatcacgtgtccatggaggtgggggcaagcgtgagctggcacgcaccccattctccccatggagggtggtcacatgtactccacctgtgtccctagatggtgagactggaggaagacttccattgggcccacttttcatgggacagtgtccatcagccaggtgctcagcccccagtgcattgaagacccagcccatcctggcaccctagtctcctgcactgagctgtgctgagcacatctggcttccactggaagttttttttgcgaggaggggcttccctgggttgggtcaatgatgagaaccttatattgtcctgaagagaggtgatgacttaaaaatcatgctcaataggattacgctgaggcccaacctaggggagaattttggaagaggattatgggatcccaaggtccccaacaaggccactgcattttgggctagagacctggaggctcttaagagcatctggaggtggcccaacctgtctctgctccttcgtgatgcagcccaggctccctgtgtgggattggtgttggtgctccatttccctggcatggggaatgttggtggctccttcctgagctcctgttctcgccttgtgtctttcagtgagctctgccacccaggacatccccctggcattgactggcataggtgagtggatcctgctggggtcattggtcatgagccagaccatgtggagtccccaaggttcagcctttgggccacaaaggttttctcagggagcagacctgaatttcacccgggagggaagggttgtcctggagggtggcttttttgggaacctaacccaaggaggacccattccctgaagccctgcaggggtgatggtggtccaggcaggaaagggttccttctgctcaatgcagggcaacacccagctaagatgcggtgatctggtaaggcctccagaactgcccaggccatatgaggggggccagaggggctacagggtgccacaggggtaacaggggagctcttggtccttggagtgtggtgcctgaatggacccatgggattgggttgtgtagattcaaggatcacaccctacccacagccctgaatgtgactcttcctcttgagcaatgggccatctcagtgggtctcctggagcctggtcggggggtggtgttgtttccctggggttgggtctaggtggagctctggctggtacctggtggtgctgcagatcatccctgggacttttgggctttgggccaagggacagcccgcatcccgcttggggtcagcctatctccatggtcaagtgtcatgaagggtggccccctgaggtgagaaggcctactgtcaagggatctgtcccaggattttgttgctcaggctgcttctgagccatggcgtcacatccacagtggcagggtcttgggcctgagcatagcagagggcaccatgtcgttctggattggccaagtatggcctagctctgtgtgcgggaggtgtgcagagcatggacggtgacccaccagagaaagcacgtggccctgcatcccaggctgtgatttccaatgcacacttggagagggtgtcttgatttgtgcccaccatgcccaggggcagactacgtgatagccaggtctgcagcctgagccacgtgtggcatctgggccagccaccttggttggccgtgagctggctggtggccttggcctgtggccagtggtgccagggcggccctggggtcgtgtgggggagccatgttgggcagtggaggtgctgtcagggcctgcagtacagtgcagcctgggcatttggcctgagcccagtcacccgcctgctctgttcctccagggtttggtggcggggagggccaagggtgccccgcatccgtgacgactcagtaattcacatggatcacatcacatttttccttggagggcaagcaaatgtgagcccccaccccccactgccttccccacatggaggtcattttggacacatatcctcctctagtccccctagatggtgagactggaggaagacttgcattatgcccgctgttcctgggccagtgtcagtcagccaggttcccagctccaagtgtgctgaagctcaggcccttcctggcaccctcagctcctgcactgagctggggttacatccgggttccactgtttgcatgcaaagggaggggtgtaccctgggttgggctgatgatgagaaccttatattgtcctgaaaaaaggtgatgacttaaacatcatgcttaatagtattatgctgaagcccaagctaggtgagaattttggaagatgaggctgggatcctgagatccccggcagggccacacttattttgggctggaaaatttcaagacctgaagcgcatcttgtgggggggcaatcctgtctctgtgcttctccgtgaggcagcccaggctcccgttgcgggcttggtgcccatgcttcgcttccccaggggtggggcaatttggcatcctcttcctgagccctccttcttccctgtgtctttcagtaagttcttcttcccaggtggggcccctggctttgagcggcatgggtcagagcatcctgtcagggtcatgggccatgagtgaggcagcgtcggttcgtcgagagtcagcctttgggccactggggttttctcagggagccaacaggttacccatcaggagggatggttcctccaggagggtcgcttccttgggaatctgactctaggagcatgcatttcctgaagctctgcagggtgatggtggtccaggcgggaaagggttccttcagcccaacataatgcagttcccagctgaaactcggtggcagatcaggctcaaggactgctcgggccacatgatgggggccacaggggatttggggtgccctgggggtcatgctggagttcttgccccaagaggggtgggtgatagggcccatgcgatcaggctgcggagattcaaggaatgctacccacccatggccctgaatgggactgaccctgttgaacaatgggccattttaatggtgcctccttgagcctggtaggtagtggcattgtttcaatgcggttgggtcaaggctgaactctggctggtgcccggtggtgcccacatgggctttgggccaagggggcagctcatatccctctggggtcaccccttccttgagattcagtggcaggaagggtgttctcctgaggtggaagacctgctgtcaagggacttgtcctgggaatttgccacctaggctgcttctgagccatggagtcacctccagggtggcagggtcttgagcccagggaaaggagacaccatatggtttctgactgggagtgtggcctggatctgtgtgtgggaggtgtgcagggcatggatggggacccatcagagaaagcatgtggccctgtaggcccccaggctttgatttctggtgtgtgcttgcagtgggcaccagggcagccctaccatgcccaggggcaggctctgtgactgccaggtctggagtggctcctggcatgaagaccacgctccttggttggccatgagcaggttggttgccctagcctgtgccctgtggtcctggtgcagccctggggttgtttggggagacagcaccggcagtggaggtgctctcaaggcctgcagtagggcgcagcccaggtgtcttgcccgagcccagtaacccacttgccctgtctttccagggtttggtggcagggagggccaactgtgccccgcatctgaaatggatcagtgttctgcatggatcccatcacaagggtatcccctacccaacttctgtgcattgttatgtgaggccctggttggacacatgtcctcctcctgtccctgcagatgctgaccctggaggaagacttgtgtcgggcccccaggccccaggccagtgtccatcagccaggtgctcagctcccagtgcgctgaagctcagacccttcctggcaccctggtgtccagcactgagctgtggtgagcccatccgggtcctgctggatgcatgaatggggaggggcctgcccagagttgggtcaatgatgagaaccttacattgtcctgaagagagatgatgacttaaaaatcatgctcaataggattacgctgaggcccagcctaggtgagaattttggaagaggacggctgggatcctgagatcccagtcagggtcactgtattttgggctggagacctggaggccctaaagggcatctggaagggggcccagccctgtctctgtgctcctccgtgaggcaacccacactccctgtgtgggcttggtgttggtgctctggttccccagggacagggcgggttggtggctccttcctgagcccctattctccctttgtgtctttcagtgagcttttccacccaggtgggccccctggcattgaccgacataggtgagtgcatcctactgggataatgggccactagatagtctgtgtggggttgctgaggtttagcctttgggccacaagggttttctcaggcagccaacctgaattcccacctgggagagatgggttctccaggaggcagcttttttgggaatcagacccaaggaggacaccttccctgaagccctgcaggggtgatggtggtccaggcaggaaagggttccttctgcccaactcgaggcccaatgcagggcagtgcccccctgagaattggtgctctggtaagggctcgaggactgcccaggccacatgaaggaggtcagaggggctatggggaaccatgggggcaacaggggagctcttggcccccggagtgtggtacatgaatgggcccatggggttgggtcacggagattcaaggattgcatctcacccacggccctgaatgcgaccatccctgttgagcaatgggccatcccagtgggtctcctggggcctggtgtggggtggcattgttttcctggggttgggtcaaggcggagctctgactggtacctgatggtgctgcaggtcagccctgggactcgtgggctttgggccaagaggcagcccgcatcctgctgggggtcgccttttctccgaggttgagtgtcgtgaaggatggcctcctgaggtgggaaggcccactgtcaaggcacctagactaggaattgttcctcaggctgcttgggagccatggtgtcacctccagggtggcgggatcttgggactaagcaaaggaggggacactacgtggctcccaattggccatgtgtggctggctctgtgcctgggaagtgtgtggagtatggatggggacccaccagagaaagcaagtggccctgcagtcccagaatgtaatttctggtgtaggcttggagagggtgcctatatgcatgcccaccatgcccaggagcaggctatgccacttcagggtctgcaggggcacctggcatatgggccagcctccttcgttggctgtgacctgggtggtggcctgtgcttgtgcccagtggtcccagggtagccctggggtcgtgcaggggagccgttttgggcagtggatgtgctctcgggtcctacagtacagcacagcctgggtgttttgctcaagcccagtgacctgcctgccttgtccttcccgggttcagtggcagggagagccaagggtgccccacattggtgacgagtcagtattttgcatggatctcatcacacgtgaccatgaaggacgagcaagcatgagctggcaccagccacccccttctccgcgtggagggtggtcacatgtcctccacctgtgcccccagatgatgagactggaggaagacttccattgggcccacttttcctgggccagtgtccatcagccaggtgctcagcccctggtgtactgaagctccagcctttcctggtgccccagtcttctgcactaagctgtggtgagcacatccgggttccactggatttttgtgcggggagggggttttctacgttgggtcaatgatgagaatcttatattgtcctgaagagaggtgatgacttaaaaatcatgctcaataggattacgctgaggcccagcctaggtgagaattttggaaggggattatgggatgctgaggtcctcagcaaggccactgtattttgggctagagacctggaggctctgaagggcatctggaggtggcccaacctgtctctgcgctcctccatgaggcagcccaggctccctgtgcagggttggtgttggtgctccggttctctggtgtggggaatgttggtggctccttcctgagccctcattctcaccttgtgtctttcagtgagctcttccacccaggagggccccttggcattgactggcataggtgagtggatcctgctggggtcattggtcatgagctagaccgtgtggggtccccgaggttcagccttcagtccacaagggttttctcggggagcagacctgaatttccacctgggagggaagggttctcctggagggtagtttttttgggaatctagcccaaggaggatgccttccctgaagccctgcaagggtgatggtggtccaggcaggagagggttccttctgcccaatgcaggacagctcccagttgagactgagtgctccagtaaggtctccaggactgcccaggccacatgaaggaggccagaggggctatggagcaccactggggtaataggggagctcttggtccctggagtgtggtgtgtgaatggggtttggtcacggagattcaaggatcacaccccacccatggccctgaaagtgaccatccctattgagcaatgggccatcccaccgggtctcctggagtctggtgcagggcgacgttttttccctggggttgggtcaaggtggagctctggtacctggtcatgctgcaggtcagccttgggactcgtaagctttgggccaagggacagctggcatcctgctggggggtcaccctatctctggggtcgaatgtcacgaagggtggcctcctgaggtgggaaggcccattgtcgagggacctgtcccaggattttgttgctcaggcttcttctgagccatggtatcacctccagggtgacagggtcttgggcccaagcaaaggagagggcaccatgtcgctctgagttggccaagtgtagcctagctctgtggtgggaggtgtgcagagcatggctggtgacccaccagagaaagcacgtggccctgcagccccaagctgtgatttccagtacatgcttagagagggcatcttgatgtgagcccaccacacccaggggcagactacgtgactgccaggactgcagtggcacatggcatctgggccagccttcttggttggccatgacctagctaatggccctggcccgtgccttggggtcatgcaggggagccctcttggacagtggaggtcctctcagggcctgcaatatggcgcaggctgggcatttggcccgagcccagtgacctacctgccctgtccttccagggttgggtggcagggagggacaagggtgccccatgtcagtgacaagtcagtatttcacatggatctcatcacatgtgcccatggaggacgagcaagcgggaactggcacccgctacttccttccctacatagaggccattttggacacatgttctccacctctctcccagatggtgagacgggaggaagacttgtgttgggcctgctgttcctgggccagtggccatcagcctgttgcccagcccctgaggcgctgaagctcaggcccttcctcacaccctggtctcctgcactagctgtggtgagcacatccgggtcccgctggatgcatgcaagaggtggttggtctcgtgggttgggtcgatgatgagaaccttatattttcctgaagagaggtgatgacttaaaaatcatgctcaataggattacgctgaggcccagcctaggtgagaactttggaagaggatgctgggatcctgagatcccaggcagggccactatattttggggtggagaactcaaggccctgaagggcctctcagggggtgccaccctgtctctgtgcccctctgtaaggcagcccaggctccctgtgtgggcttggtgtctggtctctggttccccgggggcggggcatgttggtggctccttcctgagcccgctttctcccccatgtctttcagtgagctcttctgcccaggtggggcccatggccttacacgacataggtgagtgtatcctgttggggtcatgggccatgaatgaggcagcatggggccatttagggtcagccttcgggccactagagttttctcagggtgcgaccggggagggaacatttctccagtagggtcacttccttggaaatctgatcctaggagcacgcatttcttgaagtcctgcaaaggtgatggtggtccagacaggagagggttccttcagcccaacatagcgcagctcccagctgaaactcggaaggctggtcaggcctcgaggactgcccaggccacatgaggtgggcaacaggggctctggggcaacctgggggtcacgctggagttctccctctctggacagtggtgggtgattgggcccacgcagacaggccccggagattcaggactgcaccccatccatggccctgaatgggaccgaccctgttgagtaatgggccatctcagtggtacctccttgagcctggtagggggagacactgtttcaatgcagttgaatcaaggctgagctctggctggtgcctggtggtgctgcaggcagccctgagggttgtggtctttgggacaaggggtagctcacatcccactggggtcaccccttccttgagattcagtggccaaaagggtgtcctcctgagatgggaagtcctgctgtcgagggacctgttctgggaatttgccacacaggctgcttctgagccacggggtcacctccagggtggtagggtcttgatcccaaggaaaggagaggacaccatgtggtttcttattgaccaagtgtggcctggctttgtgtgtgggaggtgtaacgcaagatgaggacccaccagagaaagcacgtggctctggaggcccccagggtttgatttccggtgtgtgcttggagagggcaccaggttgggcacccaccacgcccagggcaggctctgcgactgccaggtctgcattggctcctggtgtcagggccagactcattggttggccatgagcaggttggttgccctgacccatccccatggtcccagtgtggccctggggtttttcaggggagccggctcaagcagtggaggtgcgcttagggcctgcagtagggaacagcccaggcatcttgcctgagcccagtaacctgcttgccttgtctttccagggtttggtggccgggagggccaatgatgctctgcgtctgtgttggatcagtgttcttcatggattccatcacgtgggtatcccctacccaccttccccttgctcatacgtgaggccctggttggacacatgtccttctcctgtcaccccagatggtgagccttgaggaagacttgtattgggcccccacaggccccaggccagtgtccgtcagcctggtgctcacccccagtgtgctgaagctcagaccctccgtggcaccctggtctcctgcactgagctgtggtgagcacattcaggttccgctggatgcatgcatggagaggggcttgcccttggttgggtcagtgatgagaaccttctattgtcctgaagagaggtgatgacttaaaaatcatgctcaataggattacgctgaggcccaacctaggtgagtatgttggaagaagacactgggatcccgagatctccaacagggcactgtattttgggttggagacctggaggccctgaagggcatctggaaggaggcccaaccctgtctctgtgctcttccttggggcagcccaggctacctctgcgggcttggtgttggtgctccagttccctggggccagggaacattgctggctccttcctgagcccccattcctcccttgtgtctttcagtgaacttttcctcccagatgggccccctggcattgactggcataggtgagtgcatcctgctggggtcatgggccatcagccaggccatgtggggtcgctgaggttcagcctttgggccacaagggttttctcaggcagccgacctgaattcccacctgggagagacgggttctccaggagggcagcttttttgggaattagacccaaggaggatacctgaagccctgcaggggtgacggtggtccaggcaggaaagggttccatcagcccgggcaaggcagctcctagctgacactcggtgttcgggtcagtccttgaggactgcctgaacacgagggggtccagaggggctctgggtgccacgggggtcacatgggagctctcagtccctagagtgtggtgtgtgaatggacccttagggttaggtcttggagataaaaggattgtgctccacccatggcctggaatgtgaccgtccctgttgagtaatgggccattccagtgggtctcctggagcctgatgggaggtggcgttgtcttcctggggttgagtcaaggcagagctctggcccatacctggttgtgctgcaggtcagccctgggactcgtgggctttgggccgaggggcagcccgcatcccgctggagattaccctatctctgagatcaagcgtcatagagggtgacctcctgagaagggaaggcccactgtcgagggacctttcctaggattttgttgctcaggctgcttggaatccattgtgtcacctccagggtgatggaggtttgggcccaagcaatggagagggcaccatgtgtctcctgattgttcaagtgtgtcggagatatgcggggtatggatggtggcccaccaaagaaagcatgtgactcaagctgtgatttccagtgcatgcttagagagggcgcctgtatgcgtgccccacatgcccggggtaggctatgcaactgccaggtctgcagtggcgcatggcatcttggccagccttcttggttggccatgacctggctaatgaccctggcctgtgcccagtgatcccagggaagccctggtgtcatgcagcggaggcctcttgggcagtggaggtgctcttaaagcctgcagcatggcacggcctgggcatttatcccaagcccaatgaactacctgccctgtccttccagggttcggtggcaggcagggccaagggtgccccgtgttggtgacaagtcagtatttcacatggctctcatcacatgtgtccgtggaagataagcaagtgtgaactggcacgtcctacctccttccctacatggaggtaactttggacacatgttctccacctctctcccagatggtgagaccggaggaagacttgcattgggcctgttgttcctgggccagtggccatcagcctgttgcccagcccctgatgcgctgaagctcgggcccttcctcgcgccctggtctcctgcactgagctgtggtgagcacatccgggtcctgctggatgcatgcacgaggtgattgatcccctcagttgggtcgatgatgagaaccttatattgttctgaagagaggtgatgacttaaaaatcatgctcaataggattacgctgaggcccagcctaggtgagaattttggaagaggatgctgggatcccgagatccctggcagggccactatattttgggctggagaacttgaggccctgaagggcatctcaggggggtccaaccctatctctgtgcccctccatgaggcagcccaggctccctgtgtgggcatggtgtccgggctctggttccctgggggtggggcatgttggtggctccttcctgagcctgctttctcccccatgtctttcagtgagctcttctgcccaggtgaggcccatggccttaagcggcataggtgagtgtatcctgttggggtcatgggccatgaatgaggcagcatggggccatttatggtcagccttcgggccactagggttttctcagggtgcgaccggggagggaatgtttctccagtagggtcacttccttgggaatctgaccctaggagcatgcatttcgtgaagccctgcaaaggtgatggtggtcccggcaggaaagggttccttcagcccaacatagagcagctcccagctgaaactcagcgggctggtcaggcctcgaggactgcccaggccacatgaggtgggcaacaggggctctggggcaacctgggggtcacgctggagttctccctctctggacagtggtgggtgattgggcccacgcagacaggccccggagattcaggactgcaccccatccatggccctgaatgggaccgaccctgttgagtaatgggccatctcagtggtacctccttgagcctggtagggggaggcactgtttcaatgcagttgaatcaaggctgagctctggctggtgcctggtggtgctgcaggcagccctgggggttgtggtctttgggacaaggggccagctcacgtccctctggggtctccccttccttgagattcagtggccgaaaggatgtcctcctgaggtgggaagtcctgctgtcgagggacctgttctgggaatttgccacacaggctgcttctgagccacggggtcacctccagggtggtagggtcttgatcccaaggaaaggagaggacaccatgtggtttcttattgaccaagtgggacctggctctgtgtgtgggaggtgtagggcaagatgaggacccaccagagaaagtacgtggccctggaggcccccaggctttgatttctggtgtgtgcttggagagggcaccagggtgggcacccaccatgcccagggtaggctctgcaatggccaggtctgcattggctcctggtgttagggccagactcgctggttggccatgagcaggttggttgccctcacccgtccccatagtcccagtgcggccctggggttgttcgggggagccggcccgagcagtggaggtgctgttggggcctgcagtagggcatcttgccccagcccagtgacccaccttccctgtctttccaaggtttggtggccagcagggccaatggtgccctgtgtctgtgatgagtcagtgttcttcatggattccatcacatgggtatcccctacccacattcccctcgctcatatgtgaggccctggttggacacatgtccttctcctgtcaccaccccagatggtgagcctggaggaagacttgtattgggaccctacaggccccaggccagtgtctgtcagcctggtgctcatcccccagtgcgctgaagctcagaccctccctggcaccctggtctcctgcactgagctgtggtgagcacatccaggttctgctggatgcatgcatggggagggccttgattgggtcaatgatgagaaccttatattgtcctgaagagaggtgatgacttaaaaatcatgctcaataggattacgctgaggcccaggctaggtgagaattttggaagggagcgctgggatgccaagatccccggcagggccactagccactgtattttatactgaagaacttgaggccccaaagggcatctcaggggtcctagccctgtctctgtgcccctccgtgaggcagcccaggctccctgtgttgacttggtgtccaggctctgcttcctggggatagggcatgttggcagctccttcctgagcccacgttctcccctgtgtcttctattgagctcttctgcccaggtggggcccatgcccttaagcagtataggtgagtgtatcctgttgcggtcatgggccataaatgaggcagtgtggggtcatcgagggtcagccttcaggccactagcgttttctccaggtgcccccggggagggaacattgctccagtagggtcacttccttgggaatctgaccctaggagcacgcatttcctgaagccctgcaaaggtgatggtggtccaggcaggaaagcgttccttcagcccaacatacagcagctcccagctgaaatttggtgggctcgtcaggccttgaggactgcccaggcttcatgatgggggacacagaagctctggggctccctggggctcacactggagttctccctccctgaagagtggtgggtgattgggcccacagtcaggtggcggagattcaaggaatgcccccacccctggccctgaatgggaccaactctgttgagtaatgggccatcttaatggcgcctccttgagcctgttgggggtgggttgctgtttcaataaggttgagtcaaagctgagctctgactgggcgcctggtggtactgcaggccggcccttccttgggccaagggggcagcttgtgtcccgctggtgtcatcccttccttgaggtccagtggtggaaagggtagcctcctatggtgggaaggcctgctgctcagggacctgtcctggaaatttgccatccaggctgcttctgagccatggggtcacctccagggtagtggggtcttgagtccaagaaaaggagaggacaccatgtggttcctgattggccaaggctccttggttggccaggagcaggttggttgccctggcccatgccccatggtcctggtgtggccctggggttgttcaggggagctggcccaggccgtgaaggtgctctcagggcctgcagtagagtgcagcccaggcatcttgccccagcccagtgacccacttaccctgtctttctaaggtttggtggccaggagggccaatggtgccttgtgcccatgttgggtcagtgttctgcatggatccccatcatatgggtattccctacccaccctccctgcactgatatgtgaggccctggttggacacatgtcctcctcctgtcccttcagatggtgagtctggaggaagacttgtgttggacccccacaggccccaggccagtgtccatcagcctggtgctaatcccccagtgctctggagtttggaccctccctggcaccctggtctcctgcactgagctgtggtgagcacatggatgcatgcttggggtgggggttgcccttggttgggtcaatgatgagaaccttatattgtcctgaagagcggtgatgacttaaaaatcatgctcaataggattacgctgaggcccagcctaggtgagaattttggaagaggacactgggctccccggaatggccactgtattttgcgctggacacctgcaggccctgagggccatctggaaggtccaaccctgtctctctgctcctccaggaggcagcccaggctccctgagggggcttggtgtcgggctcctgttccccggggttggggccggttggtggctccttcctgagcccccattctccccttgtgtctttcagtgagctcttccacccgggcgggcccccggcattgactggcataggtgagtggatcctgcttgtgtcatgggccatgaaccggtccatgtggggtcaccaagggtcagcctttgggataagagggtttcctcagggaaccaacctgaattcccatcagagaggtatgggttctccaggagggccgcttttttgggaattggacccaaggaggataacttccctgatctctgcaggagtgacggtggtccaggcaggaaagagttccttcagccccacccagcgcagcgttctgttgagactcagtgctcaggtcaggcctggaggatttctcaggccatctgagggggttagaggggagctctttgtccatggagtgtggtgcttgaatctgcccttggggttgggtcatggagattcaaggatggtgcctcacccatggccctgattggaattgtccattgagaaatacgccattccagtgggcctcctggagcctgatggggggtggtgtttttttccctggggttgagtcgttgatggggactcaccatagaaagcatgtggctctggaggcccccaggctttgatttccagtgcctgcttggagtgggcaccagggccggcacccaccatgcccaagggcaggctctgcgactgccaggtctgcattggctcctggcatcaggaccagcctccttggttggccatcagctgcttggttgccctggcccatgccccgtggtcctggtgtggccctggggtggttgagtggagctggcccaggcagtggaggtgctcttggggcctgcagtagggtgcagcccaggcatcttgcctgatcccagtgacctgcttgccctgtctttccaggattttgtggcagggagggccaagggtgccctgcatccgtgatgggtcagtgttccacgtggatcccattacgtgggtccatagaggatgagcatgcgtgagccagcgcccccttcccacttcttctcatggagctgtgaggccctgtgtggacagctgtcttcctcttgtcccaccaggtggtgagcccggaggaggacttgcgttcggcccgctggtcccagtgtgtccggaattggtgggttcttggtctcactgacttcaagaatgaagctgcggaccctcgctgtgagtgttacagctcttaaggtggcatgtctggagtttgttccttctgatgttcggatgtgttcggagtttcttccttctggtgggtttgtggtctcgctggcttcaggagtgaagctgcagaccttcgcggtgagtgttacagctcataaaagcagtgtggacccaaagagtgagcagtagcaagatttattgcaaagagtgaaagaacaaagcttccacagtttggaaggggacccaagtgggttgccactgctggctggggcagcctgcttttattctcttatctggccccacacacatcctgctgattggtagagctgagtgatctgttttgacagggcgctgattggtgcgtttacaatccctgagctagacacaaaggttctccatgtccccactagattagctagatacagagtgttgacacaaaggttctccaagtccccaccggagtagctagatacagagtgtctattggtgcattcacaaaccctgagcttgacacagagtgctgattggtgtgtttgcaaaccttgagccagatacagagcgcggattggtgtatttacagtccctgagctagacataaaggttctccaaggccccaccagagaagctagatacagagtgtcgattggtgcattcacaaaccctgagctagacacagggtgctgattggtgtgtttacaaaccttgagctagatacagagtgccgattggtgtatttacaatccctgagctagacataaaggttctccaaggccccaccagactcaggagcccagctgtcttcacccagtggatcccgcactggggctgcaggtggagctgcctgccagtcctgcgccatgcgcccgcactcctcagcccttgtgtggttgatgggactgggcaccgtggagcagggggcggcactcgtcggggaggctctggctgcacaggagcccatggagggggtgggaggctcaggcatggcaggctgcaggtcccgagccctgccccgagggaaggcagctaaggcccggtgagaaatcaagtgcagcgccggtgggctggcactgctgggggacccagtataccctctgcagctgctggcccgggtgctaagcccctcattgcctggggccggcagggccggcccgctgctccgagtgtggggcccgccaagcccacacccacccggaactccagctggcccacaagcgccccgcgcagccccggttcccacttgcgcctctccctccacacctccctgcaagctgagggagcaggctccggccttggccagcccagaaaggggctcccacagtgcagcagtgggctgaagggctcctcaagtgccaccaaagtgggagcccaggcagaggaggcgccaagagcaagtgagggctgtgaggactgccagcacgctgtcacctttcatcaggacagtgtctgtcagccaggtgcacagcccttggtgtgctggagcttgggcttttcttggcagcctggtgtcctgcattgagcggtggtgagcttgtccaggtcccgctggttacctgcacggtgacgggggtgccctcggttaggttgatgatgagaaccttattttgtctcgaggagaggttatgtgatgggtgttgtggcagctctttgtatttcattttcttcagtgctttttgtgtgttctctttgtattttttaccgctttgtgtattttactttcacaaaggcagcagcaaatgttttattggtggtggtatttttcatttttatttacttcctaatttgatagtgttttagttggaatacacagtctcattgtagttgttcagaaatatgctttttttctgtacattatatgattgtgcataatttttgccagttttttttttaattggggactacagttgaatgcactgcctatctcctactttactgaaacagattgtatggaaatctaaagacgagaaagtagacaagcacagcagaggacatttattttgatataaccacagaaattcattgatatgaccttatcatattgatggtttttcaaagaaatataaaatctttcgataatggattgtctcagttgcgtttctccttatatcatgttttgccttcaatacaaattgcaatgcttagaaatttaaagaacaccttctgaagtggaagatctagctaaagaattagattggtattggctgaaatgttgcagtaatcacatgatagggttaatacaacatgagttacatgctgagtgctctaaggtagaaagtaggtaacaaggaataacaggtaggtaatgtaagtcgggagaaggaaactctaggacagaatcattagaaaaggcttgacgtcaacagcagtgtatagaatttacagtgtatagaatttacagtgcatagaatttacaataagtgcctttttttgtgctcatcaatgctcttgcttatattgtaggaggagttagttcccttgaagaaatatcaatacaaatttccaaattgcaatgcagagggaaaaaagaatgagaaagtaaaaaatatccaagaactctggaagcatttcaaaaagtgtaaaaatgcatagtgacaatgtcaggagaataaggagataaaggaaaagaaatatttgacacaacaatgacttgagcactttctaaaattaatgatggacagaaacccacatgttcaggaagctcagagagcactctgcagaataaaaatattttacaaatcgacacctttgcatttcatatttaaagtccagaaaatcatagactaagggaaaagcttgataggagcaataggaagaaaaccatcttacctatggagcaacaagaataaaaattgctttgaaattatcttcagaatccatgaaagcaagaagtgagtcagggtgaaatatttagtgttaaaagtaagaaacaaaaaaaagtaccaactttgaattttctgctcatcaaaattattcttcaaaagtgaaggagaaatactctctgagaccaacaaaaattgctagaacttcttgccactacaccaaccttttaataaacgtaacaagaaattcttcagaaaggataaaaatgacataggtcggaaaatctaatttacataaagaaatgaaaagcattggagaagaaatatatgaagaaaaattaaatatatatgtattttttgattcataattgatctttcaacaatttgttcaaaataataacaagattatatttggtgattataggttatggaaaagtgaaatgaatgacagcaatattataagtcatccggttccaagatggccgaataggaacagctccagtttacagctcccagtgtgagtgacgcagaagacagttgatttctgcatttccaactgacgtaccaggttcatctcactggggcttgttggacagtgggtgcagcccatggagtgtaagccgaagcaggacgaggcatcacctcacctgggaagtgcaagaggtcagggaattccctttcctagccaagggaagcgtgacagatggtacctggaaaattgggacactcccaccctaatactgtgcttttccaactgtcttagcaaacggcacaccaggagattatatcccgtgcctggcttggagggtcccacatccacggagccttgctcactgctagcacagtagtctgagatcaaactgcaaggcagcagtgaggctgggggaggggcatccaccattgctgaggcttgagtaggtaaacaaagcggctgggaagctcgaactgggtggagcccaccacagctcaaggaggcctgcctgcttccgtagactccacctctaggggcagggcatagcagaacaaaaggcaacagaaacttctgcagacttaaacatccctgacagctttgaagagagtagtggttctcccagtacagagtttcagatctgagaacagacaggctgcctccttaaatgggtccctgacccccaagtagcctaactgagagacacctcccagtaggggctgactgatacctcatacagctgggtgcccctctgagacgaagcttccagaggaaggatcaggcagcaacatttgccgttctgcaatatttgctgttttgcagcctccactggtgatacccaagcaaacagggtctggagtggacttccagcaaactccaacagacctgcagctgaggatcctgactgttagaaggaaaactaacaaagagaaaggacatccacaccaaaaccccatctgtacgtcaccatcatcaaagaccaaaggtagatgaaaccacaaagatggggagaaaccagagcagaaaaactgaaaattctaaaaatcagagcacctcttctcctccaaaggaatgcagctccctgccagcaatggaacaaagctggacggagaatgactttgacgagctgacagaagtaggcttcagatgatcgctaataacaaacttctctgagctaaaggatgaggttcaaacccatcgcaaagaagctaaaaaccttgaaaaaagattagatgagtggctaactagaataaacagcatagagaagaccttaaatgacctgatggagctgaaaccatggcacaagaactacgtcacacatgcacaagcttcagtagccgatttgatcaagtggaagaaagggtatcagtgattcaagatcaaatgaatgaaatgaagcaagaagagaagtttagagaaaaaagagtaaaaagaaacaaagcctaaaaaagtaaaaaagtaaaacaaagtaaaaaagaaacatgggactatgtgaaaacaccaaatctacgtctgattggtgtacttgaaagtgatggggagaatggaaccaagttggaaaacactctgcaggatattatccaggagaactcccccaacctaggaaggcaagccaacattcaagttcagaaaatacagagaatgccacaaagatactccttgagaagcgtaactccaagacacataattgacagatacaccaaagttgaaatgaaggaaaaaatattaagggaagccagagagaaaggtcaagttacccacaaagggaagcccatcaaactaacagcaaatctctcagcagaaactctacaagccagaagagagtgggggccaatattcaacattcttaaatgaaagaattttcaacccagaatttcatatccagccaaactaagcttcataattgaaggagaaataaaatactttacagacaagtaaatgctgagagattttgtcaccaccaggcctgccctacaagagctcctgaaggaagcactaagcatggaaaggaacaaccagtaccagccactgcaaaaacatgccaaattgtaaagatcactgatgctaggaagaaactgcgtcaactaacgagcaaaataaccagctaacatcacaatgacaggatcacattcacacataacaatattaaccttaaatgtaaatgggctaaattctccaattaaaagacacagactggcaaattggataaagagtcaagacccatcagtgtgctgtattcaggagacccatctcatgtgcagagacacacataggctccaaataaagggatggaggaagatctaccaagcaaatggaaaacaaaaaaaagcaggggttgcaatcctagtctctgatgaaacagactttaaaccaacaaagatcaaaagagacaaagaaggccattatgtaatagtaaaggggtcaattcaacaacaagagctaactatcctaaatatacatgcacccaatacaggagcacccagattcataaagtaagtccttacagacctacaaagagacttagactcccacacaataataatgggagactttaacaccccactgtcaacattagacagatcaacaagacagaaagttaacaaggatatccaggaattgaactcagctctacaccaagcagacctaatagacatctacagaactctccaccccaaatcaacagaatgtacattcttctcagcaccacatcgcacttattccaaaattgaccacatagttggaagtaaagcactcctcagcgaatgtaaaagaatagaaattataacaaactgtctctcagaccacagtgcaatcaaactagaactcaggattaagaaactcactcaaaaccactcaactacatggaaactgaaaaatctgctcctgaatgactactgggtacataatgaaatgaaggcagaaataaagatgttctttgaaaccaatgagaacaaagacacaacataccagaatctctgggacatatttaaagcagtgtgtagagagaaatttatagcactaaatgcccacaagagaaagcaggaaagatctaaaattgacaccctaacatcacaattaaaagaactagggaagcaagagccaacattttcaaaagctagcagaaggcaggaaataactaagattggagcagaactgaaggagatagagacacaaaaaacccttcaaaaaatcaatgaatccaggaactggttttttgaaaagataaacaaaattgatagaccactagcaagactaataaagaagaaaagagagaagaatccaatagatgcaataaaaaatgataaaggggatatcaccaccaatcccacagaaatacaaactaccatcagagaatactataaacatctctatgcaaataaactagaaaatctagaagaaatggataaattcctggacacatacaccctcccaagactaaaccaggaagaaggtgaatctctgaatagaccaataacaggctctgaaattgaggcaataattaatagcctaccaaccaaagaaagtccaggaccagagggattcacagctgaattctgccagaggtacaaagaggagctggtaccatgccttccgaaactattccagtcagtagaaaaatagggaatcctctctaactcattttataaggccagcatcatcctgataccaaagcctggcagagacacaacaaaaaagagaattttagaccaatatccctgatgaacatcgatgcaaaaatcttcaataaaatactggcaaaccgaatccagcaacacatcaaaagctaatccaccacgatcaagttggcttcatccctgggatgcaaggctggttcaacatacacaaatcaataaacataatccatcatataaacagaaccaaagacaaaaaccacatgattatctcaacagatgcagaaaaggcctcgacaaaattcaacagcgcttcatgctaaaaactctcactaaactaggtgttgatgggacgtatctcaaaataataagagctgtttatgacaaacccacagccaatgtcatactgaatgggcaaaaactggaagcattccctttgaaaactggcacaagacaggaatgccctctctcaccgctcctattcaacttagtgttggaagttctggccagggcaatcaggcaggagaaagaaataaagggtattcaattaggaaaagaggaagtcaaattgtccctgtttgcagatgacatgattgtatatttagaaaaccccatcgtctcagcccaaaatctccttaagctgataagcaacttcagcaaagtctcagaatacaaaatcaatgtgcaaaaatcacaagcattcctatacaccaataacagacaaacagagagtgaaatcatgagtgaactcccattcacaattgcttcaaagaataaaatacctaggaatccaacttacaagggatgtgaaggacctcttcaaggagaactacaaaccactgctcaacgaaataaaagaggacacaaacaaatggaagaacattccatgctcatggataggaagaatcagtatcgtgaaaatggccatactgcccaaggtaatttatagattcaatgccatccccatcaagctaccaatgactttcttcacagaattggaaaaaactactttaaagttcatatggaaccgaaaaagagcctgcattgccaagtcaatcctaagccaaaaggacaaagctggaggcatcacactacctgacttcaaactatactacgaggctacagtaaccaaaacagcatggtactggtaccaaaacagagatatagaccagtggaacagaacagacccctcagaaataataccacatgtctacaaccatctgatctttgacaaacctgacaaaaataagaaatggggaaaggattccctatttaataaatggtgctgagaaaactggctagccatatgtaaaaagctgaaactggatcccttccttacaccttatacaaaaattaattcaagatggattaaagacctaaatgttagacctaaaaccataaaaaccctagaagaaaacctaggcaataccattcaggacataggcatgggcaaggacttcatgactaaaacaccaaaagcaatggcaacaaaagccaaaataaacaaatgggatctaattaaactaaagagtttctacacagcaaaagaaactaccatcagagtgaacaggcaacctacagaatgggagaaaatttttacaatctactcatctgacaaagggctgatatccagaatctagaaagaagttaaacaaatttacaagaaaaaaatcaaccccatcaacaagtgggcaaaggatatgaacagacacttctcaaaagaagacatttatgcagccaaagacacatgaaaaaatgctcatcatcactggccatcagagaaatgcgaatcaaagccacaatgagatcccatctcacaccagttagaatggcaatcattaaaaagtcaggaaacaacaggtgctggacaggatgtggagaaataggaatacttttacactgttggtgggatggtaaactagttccaccattgtagaagacagtgtggcaattcctcaaggatctagaactagaaataccatttgtcccagccatcccattactgggtacatacccaaaggattatatatcatgctactataaagacacatgcacatgtatgttcattgtggcactattcacagtagcaaagacttggaaccaactcaaatgtccatcaacgatagactggattaagaaaatgtggcacatatacaccatggaatactttgcagccataaaaatggatgagttcaagtcttttgtagggacatggatgaagctggaaaccatcattctcagcaaactatcgcaaggacaaaaaaccaaacactgcatgttctcactcataggtgggaattgaacggtgagaacacttggacacagggtggggaacatcacacacgagtggctgccgtggggtggggggaggggggagggatagcattaggagatatacctaatgtaaatgacgagttaacgggtgcagcacaccaacatggcacatgtatacatatgtaacaaacctgcacgttttgcacatgtaccctagaacttaaagtatgaaatatatatatatatatatatatatagtcatgagggagagaaaatactcttagttacttggactaacccacgaagtaggacagtataatttgaaaaaggacttggattagttgtaaacgtataatgtaatatccagcacagccactaaaaatgattttgaaaaaagcagtataatcaatatactaacagaagaaaaagcagaatcaggccagacatggtgactcatgtctgtaatcccagcactttgggatgctgaggcaggaagatcacttgagccccggagttcaagtccagcctggcaacgaataagagcccatctctaccctccaccccccccaaaaaaaaaaagaaaaagaaaaagaggccgggcacggtggcttactcctgtaatcccagcactttgggaggccgaggtgggtggatcgcgaggtcaggagatcaagaccatcctgcctaacacggtgaaaccccgtctctactaaaaatacaaaaaaaaaaaaaaaatagctgggcatggtggcgggcgcctgtagtcccagctactcaggaggctgaggcaggagaatggtgtgaacctgggaggcagagcttgcagtgagccgagattgcgccactgcactccagcctgggagacagccgcaagattctgtctcaaaaaaacaacaacaacaacaaaaaaaaacaaaaacaaaaacaaagaaaacgaaaagaaaaagcagaatctttttttttttttttttttttttttttttgagatggagttttggtcttgttgcccgggctggagtgcaatggcacgatttcagctcaccacaacctccacctcccaggttcaagcaattctccgggctcagcctcccgaatagctgggattacaggtgtgcaccaccatgcctggctaattttttgtatgaaaaagcagaatcttatacaatgctcaattaaaatcagggagaggagaaaaagagtagaataccaaaaaagtaacaaattacaaggttgatataaatagaaaacaatcacaaatactgagtatactaatctaagtatatcaataatcactttaaacatcgcttaaaagacagagattgtcagtggattaagaacaagacccaaatatatgttgtctatgagaaatgcactttaaatagctgggcatggtggtgtgcacctgcagtccttaggaggccaaggcggagtgtcacctgagcccaggattttgggactacagtgagctatgatcatgccactgcactctagcctggttgacagaataagacctcatctctatagaaaaaagtaagtaaatctactttataaaggcacaaatatattcacagtaaaggaatagagaaagatatcccatgctaacattaatcaaaataaagtgaaatagccatattaatttcagacacaaccgactttagaacaaggaatattatcagagatcaaaagggacattccttaatgataaagggatcagttttctaagaagacataacaatccttaatgtgtacgcactgaacagcagatcatcaaagtacatgaagctaaatctgatagaactgcatagagaaatagataaattcactcttagagttggaggcattggcatccctctatcagaaatggacaggtttagcaagcagaaaatcagtaagggcataacatgatcaatcatgatcaaaaacttgattcagtcaactatatgtaactgacatctatggaccactttacccaacaacagcagatgacacattttctcacacacacatgaaacattcagcagaatagaccacattctgggctagaaaatgcacctcaacaaatttaaaagaatagaaatcatacaaagtatgttctcagactacaatggaatgaaagtaaacaaaaatcactaaaaaagatagctgaaaaacatgaaaatacttggaggttaaacaacagactgaaataactgaaggtcacagaagtctcaagataaatttttaaaatatttcaaactaaatggaattgaaagcacaacttatcaaaaatttaggaacgcaacaaaggcagtgcctactgggaaatttataccactgaatgtatgttaaaaagaacaaagatctaaataaataatccttcccattggaaagctagggggaaaattcaaattaaattcaaagtaagcaagtaaaagaaataataaaatatgaatacaattctgagtggcctgatatctgatttctggaagctgagttttatgtggctttagaagtttcatgttatgagtgtgaaatggcaagtgaccatggcgattgcttctgatatggccacctcaattacatagagaagggatgatcagtggcatcatcccagttctgcatgataccacaccagtgattctccaaagtcaagaaacctgtatatgttgtccttgtgtttggtgaatctgtggtctatagactacataatgggctatttctggggtactgaccattttgctccattaaggaaaaccatgcatgagccatataactatgggaaacaatgttttcctttttgacacctggcctactctcttcttaggtgagagcaaaggaagagcaccacttggcctgctgcacccagggcaatttataccatccagatatccagcactgaatatgtagcaactccaatccaccatagaacattaatctatctctgataagcatgctaatatctatgattctgcctttgtggggagatgactccatagattaaccccctggcttggggcaatatggagatgttatattgtcttcgacagggaagatgacataaaaattatgttcaataggattatgtggagacttaacataagtgacaaacctaagacaagaagttagaaatccttgtttcttttaggtgaataattttggagcttgagaaaaagacaccattgtggcgaacttgctttgccaaaccctgtgtatgcatttttgggtaaacaacacattttcatcctttaagcattgtttccattatttataattcctaatagtatgagacatttaggactcattccttggttcttcttctctcattgtatctttcattgtatctttcagtgctgcaagagatgcaatcatcccattaatgaaagcagagtgactgccttcgtcacacatacattcaaaatagatcactaggctagtgatgattctctcttaaggctattatgtttactcatttaaaaaatctggatctcagtatagattttcttctcagtgagtgactgacctatctgatatgatgctatgacaaaatctttactttttacctgaatttggtgggattgaaagttgagaacatattaatttattttctgtttcacttgattaataatgcccaatctaacatgcagtgttcacttgggtacatatatacaactacatcaaattttttcttgttttaccttaaaagtgttaagatattgtaatggtatggccagtttactcactggtaaacttttcataacagaaatatcatgccagtggacacaaatatgtaacatcagtaatttgctttcctcatgaaacagagaactgaataagttgatgggtaaactagtatctgaattataaaccccaaatatcatgtgacttcatcattttcagtataatgaaaatacaggctgtgaatgcttccagttggtcctgaaagggcctttgaggtgagtaggttcaccaaagcggcaacatatgtgtcccagtgggcattctactcctcagtaagtgacttgacattctttatggcttcctcatgattaaaacaagtctttttattgaaattttactgaaataatgatgaaacaatgtaatattcttttcatctacatataagagtagacatagattaacctctaccttttttttttttttttttgagacggagtctcgctctgttgcccaggctagagtacagtggtgcgatctcagctcattgcaacctctgcctcccaggttcaagcgattctcctgcctcagcctcctgagtagctgggattacaggcacgtgctaccattcctggctaatttttttgtaattttagtagagacatggtttcaccatgttggtcaggctgttcttgaactcctgaccttgtgatctgcccacctctgcctcccaaagtgctgggattacaggcttgagcccccgtgcctggccttcacctctacctattataatattttcatgttattccattttgtcatgtttgtgggtcatttgatgagggttcacaatatgaaaactgggcattttggccaagatgcatgtaaatgaatacaactccatgtgccctgataactgatttctagatcctgagatgcaagtggcttccagagtgtctgtattttatgaaagttcatatacagtaaaccatgctaattgttactgatacagcttccttgaatatataaagcgatgattaatgggatgttttgtttctgcagaaaaccactgaattgcttctccaaagccaagtgacctgtatgatttgtccttattatattttgtgaaccagtggactaaaacctttataatgggtcatttttttagtattgtcaatattgctcaattaacaaacatgactcatatacacaggtgagatgtagttctccctttgacatttgctctacatctgtcccttagatgatgatatggaagaaaagcactctttggcctgttgtgactgggacagttgacagcacccaggtgtcctttaatgaaaatgctcttgacaccaatgcatcctagcatcacagcttcaggaagccttctcaagtgtgcatggggagtactatgtctttcatcaataatgaaatcttctgatttggtgagaaataatgccttaaaattacactcaataggattatgctgaggctcagcctacctagatgaaaatgctgaaacagataatagtgacttcttatttcccttcaaggggaattttattttgaacttgaaataacaaaatctttatgggtaacttgtttgcccaactgtatcagtgcacctataaatgaacaaaaaattctcagcctataagctttgcttcagtgacctttaatttctaatagtaaggtatattcaggtctcattctttggctttttctcttattgtgtatttcagtaagacatgctgccaagagatgtgccattctattataaaagatcagtagcttcctttaccgacgtgtatattctatctagaacattgagctatggaagactcccacctaagggaattagttttacaccttcaggtaatctgaacttcaatatgagttttgctttatgatataatccttatgacaaaatctttactttttatctgaacttcatacaatcaatattgaagattttttaaattcactgttacctagtccaatatttaatatacatttggctgtagatacatgactagacaaaaattttttatattattattgtgggggctctaacagtgtttcactgttttgagttactgatgaactttctgtaactgtgagaccatggtaatggaagagaccgtggtaatggggaggagaatgtgaggatcagtatgtactttgcctataaaacttaaatatttagtaatttgatcaccaaattgatactcaaatttcagttcctatcatgtgtaaaccaaaaagtatctgagatttagaagtttatttagaagtggattttgccaaggttaagcacatgctcagaagaaataaacccaaaatcacagaaaccgtctggggtctatgtctttctccaaagatgattttgagggcttcagtgtttaaaggggaaaagtgggctggagggggaaaagggagggtatgataatccgtatgttccaagacaaaaggagcaggtaggggtatagtcaataatgtattcatctcatgctcagtaaatcagcactttacataagataaggtgaacatagagaagctacctgtggagatctttaacctttaatctgtagctatctgcttaagatcaaaaggaaattcttgcatgactcagctttcagcttattttttttttctcttggcgtagtaaattggggtcccaagtttattttcctttcacacatgtgaccccactattttcagaataatgaaagcacatgttagaaatgatgacacttattcctaatatggactttgaaatgactgggttcacaaatttagaatcatggatctcattataggtttttattttcaatgagtgactggcctacttgatggaattctttcaaaaaatgttcactttttactgagattatattaaaataatgttggaatgtgttcagtcatgtttaccatctctatagtatgatatatgataatatcgcagattactcagtgtcaaaaacctggcataccttgtccttatgatgttgatgagtcaatggtctagagccttagtaatatgtctttttgtggtagtacggaccgtatctgtccactaagaaatagcctgtgtaagccatatacacatgaacaatttatgtctgattttgaactatggtcccattcctatgcctgtagataaagactgctgagaagagcaccctctggtgttgtcacagaggcaagtgctaccgcacaggcatgctgcagtgaatttaactgatcctctgtccctgcaaccgttgtttaaggatgctattctggtaagggtttacaagacaacgtaaatatatgtataaagagtatcttcaggagacgtaataatgtaaaaatcatgctcaatagaattaagctgaggctcaaaatagtgccaatccttgcaagaaggtgtgagaagtcattagatttggggggtatgggttattccattttgaacttggtaggaagaaacccttgggggaatcttacatcatttgcctctacctacttctaaaggaacttttaaaattcttttatcttgtctttattcttaatactcatacattattagagagaatatcttggtttattttttcctcatggtgtgcttcagaatgttcatccagcaagaggtgtgtcattacacggattatatatatgagttctgtctttcgtaattaaaaaatttcaagtggaccatttgaatattaaacattggccttcaaaatgattacatttacctgttcatacaaaattagtgagcaatgatctattctttccaatgagtgactgatctaatgaggtcctcattaaaactccttttttttttttttttttttttgcctgaacgtactttgatttaaagatgtgacatttagtaattcattgcaatcaactctttagtgattgcacaaatagacaatccttttctattattttattttttggttgctgtgtatggtgttatgatgtttgtggatcattgaggcactactcacaactgaaagactttggcaattttagggaatggcttgagattcagctataggttacccttgattgaaattgaatacaatacaggtagtctgaaatctcatttccttatcctgagtctcactcaggtgtcattagattcattataaagaaatgcatacactatgtatgatgccagttgttactgatatgaagacgataagaagcttttatttctgcagggaaataatcccttaccaggtcatcaaaagacatgatacctatgtgcttggcccttactgtacacggggaatcagtggtctaccacagcttaagtaacgggtcatatttggagtatcacacatctcagtcttgtagaaattaggaacagcaattaggagtcatgcacatataagagatgtaatcccaccctttgactatagcctactcttgtcttttacagaaaagactgtggaggaagaaaaccctttaccctgttgttcagggagaaactgacaccactcaactgcctggcactgaaaatgtggcatccagtccactttaccatcagtgtttaaggaaaccatctctggtaagcatatttgatccaagtgtgcatatgcacatgagtgccatgggctgggtcaatgatgagatgttaccttgaagagaaatgatgacgtaaaaattaagttcagttggattacgctgaggccc

SEQ ID NO: 3 - DNA, 48 bp, synthetic construct
  misc_feature 1..48: note = shRNA artificial/synthetic sequence
  source 1..48: mol_type = other DNA
tgatgatgagaaccttatattctcgagaatataaggttctcatcatca

SEQ ID NO: 4 - DNA, 82 bp, Homo sapiens
  misc_feature 1..82: note = SNORD115-1 human genomic sequence
  source 1..82: mol_type = genomic DNA
gtgttgatgatgagaaccttatattatcctgaagagaggtgatgacttaaaaatcatgctcaataggattacgctgaggccc

SEQ ID NO: 5 - DNA, 82 bp, Homo sapiens
  misc_feature 1..82: note = SNORD115-5 human genomic sequence
  source 1..82: mol_type = genomic DNA
ggatcgatgatgagaaccttatattgtcctgaagagaggtgatgacttaaaaatcatgctcaataggattacgctgaggccc

SEQ ID NO: 6 - DNA, 82 bp, Homo sapiens
  misc_feature 1..82: note = SNORD115-9 human genomic sequence
  source 1..82: mol_type = genomic DNA
gggtcgatgatgagaaccttatattgtcctgaagagaggtgatgacttaaaaatcatgctcaataggattacgctgaggccc

SEQ ID NO: 7 - DNA, 81 bp, Homo sapiens
  misc_feature 1..81: note = SNORD115-10 human genomic sequence
  source 1..81: mol_type = genomic DNA
gggtcgatgatgagaaccttatattgtctgaagagaggtgatgacttaaaaatcatgctcaataggattacgctgaggccc

SEQ ID NO: 8 - DNA, 82 bp, Homo sapiens; identical to SEQ ID NO: 6
  misc_feature 1..82: note = SNORD115-12 human genomic sequence
  source 1..82: mol_type = genomic DNA

SEQ ID NO: 9 - DNA, 82 bp, Homo sapiens
  misc_feature 1..82: note = SNORD115-13 human genomic sequence
  source 1..82: mol_type = genomic DNA
gggtcgatgatgagaaccttatattatcctgaagagaggtgatgacttaaaaatcatgctcaataggattacgctgaggccc

SEQ ID NO: 10 - DNA, 82 bp, Homo sapiens
  misc_feature 1..82: note = SNORD115-17 human genomic sequence
  source 1..82: mol_type = genomic DNA
gggtcgatgatgagaaccttatattgtcctgaagagaggtgatgacttaaaaatcattctcaaaaggattatgctgaggccc

SEQ ID NO: 11 - DNA, 82 bp, Homo sapiens; identical to SEQ ID NO: 10
  misc_feature 1..82: note = SNORD115-18 human genomic sequence
  source 1..82: mol_type = genomic DNA

SEQ ID NO: 12 - DNA, 82 bp, Homo sapiens; identical to SEQ ID NO: 10
  misc_feature 1..82: note = SNORD115-19 human genomic sequence
  source 1..82: mol_type = genomic DNA

SEQ ID NO: 13 - DNA, 82 bp, Homo sapiens
  misc_feature 1..82: note = SNORD115-20 human genomic sequence
  source 1..82: mol_type = genomic DNA
gggtcgatgatgagaaccttatattgtcctgaagagaggtgatgacttaaaaatcatgctcaataggattatgctgaggccc

SEQ ID NO: 14 - DNA, 81 bp, Homo sapiens
  misc_feature 1..81: note = SNORD115-21 human genomic sequence
  source 1..81: mol_type = genomic DNA
gggtcgatgatgagaaccttatattttctgaagagaggtgatgacttaaaaatcatgctcaataggattacgctgaggccc

SEQ ID NO: 15 - DNA, 82 bp, Homo sapiens
  misc_feature 1..82: note = SNORD115-27 human genomic sequence
  source 1..82: mol_type = genomic DNA
gggctgatgatgagaaccttatattgtcctgaaaagaggtgatgacttaacaatcatgctcaataggattacattgaagccc

SEQ ID NO: 16 - DNA, 82 bp, Homo sapiens
  misc_feature 1..82: note = SNORD115-37 human genomic sequence
  source 1..82: mol_type = genomic DNA
gggctgatgatgagaaccttatattgtcctgaaaaaaggtgatgacttaaacatcatgcttaatagtattatgctgaagccc

SEQ ID NO: 17 - DNA, 82 bp, Homo sapiens
  misc_feature 1..82: note = SNORD115-40 human genomic sequence
  source 1..82: mol_type = genomic DNA
gggtcgatgatgagaaccttatattttcctgaagagaggtgatgacttaaaaatcatgctcaataggattacgctgaggccc

SEQ ID NO: 18 - DNA, 82 bp, Homo sapiens
  misc_feature 1..82: note = SNORD115-42 human genomic sequence
  source 1..82: mol_type = genomic DNA
gggtcgatgatgagaaccttatattgttctgaagagaggtgatgacttaaaaatcatgctcaataggattacgctgaggccc

SEQ ID NO: 19 - DNA, 21 bp, synthetic construct
  misc_feature 1..21: note = SNORD115 artificial/synthetic sequence
  source 1..21: mol_type = other DNA
caatgatgagaaccgtatatt

SEQ ID NO: 20 - DNA, 21 bp, synthetic construct
  misc_feature 1..21: note = SNORD115 artificial/synthetic sequence
  source 1..21: mol_type = other DNA
ggtaacctgttctccaaattt

SEQ ID NO: 21 - DNA, 21 bp, synthetic construct
  misc_feature 1..21: note = SNORD115 artificial/synthetic sequence
  source 1..21: mol_type = other DNA
tgatgatgagaaccttatatt

SEQ ID NO: 22 - DNA, 21 bp, synthetic construct
  misc_feature 1..21: note = SNORD115 artificial/synthetic sequence
  source 1..21: mol_type = other DNA
gccttcacctctacctattat

SEQ ID NO: 23 - DNA, 21 bp, synthetic construct
  misc_feature 1..21: note = SNORD115 artificial/synthetic sequence
  source 1..21: mol_type = other DNA
ccttcacctctacctattata

SEQ ID NO: 24 - DNA, 21 bp, synthetic construct
  misc_feature 1..21: note = SNORD115 artificial/synthetic sequence
  source 1..21: mol_type = other DNA
cttcacctctacctattataa

SEQ ID NO: 25 - DNA, 21 bp, synthetic construct
  misc_feature 1..21: note = SNORD115 artificial/synthetic sequence
  source 1..21: mol_type = other DNA
ttcacctctacctattataat

SEQ ID NO: 26 - DNA, 21 bp, synthetic construct
  misc_feature 1..21: note = SNORD115 artificial/synthetic sequence
  source 1..21: mol_type = other DNA
tcacctctacctattataata

SEQ ID NO: 27 - DNA, 21 bp, synthetic construct
  misc_feature 1..21: note = SNORD115 artificial/synthetic sequence
  source 1..21: mol_type = other DNA
cacctctacctattataatat

SEQ ID NO: 28 - DNA, 21 bp, synthetic construct
  misc_feature 1..21: note = SNORD115 artificial/synthetic sequence
  source 1..21: mol_type = other DNA
gatacagcttccttgaatata

SEQ ID NO: 29 - DNA, 21 bp, synthetic construct
  misc_feature 1..21: note = SNORD115 artificial/synthetic sequence
  source 1..21: mol_type = other DNA
atacagcttccttgaatatat

SEQ ID NO: 30 - DNA, 21 bp, synthetic construct
  misc_feature 1..21: note = SNORD115 artificial/synthetic sequence
  source 1..21: mol_type = other DNA
tacagcttccttgaatatata

SEQ ID NO: 31 - DNA, 21 bp, synthetic construct
  misc_feature 1..21: note = SNORD115 artificial/synthetic sequence
  source 1..21: mol_type = other DNA
acagcttccttgaatatataa

SEQ ID NO: 32 - DNA, 21 bp, synthetic construct
  misc_feature 1..21: note = SNORD115 artificial/synthetic sequence
  source 1..21: mol_type = other DNA
cagcttccttgaatatataaa

SEQ ID NO: 33 - DNA, 21 bp, synthetic construct
  misc_feature 1..21: note = SNORD115 artificial/synthetic sequence
  source 1..21: mol_type = other DNA
ctgtatgatttgtccttatta

SEQ ID NO: 34 - DNA, 21 bp, synthetic construct
  misc_feature 1..21: note = SNORD115 artificial/synthetic sequence
  source 1..21: mol_type = other DNA
agcacccaggtgtcctttaat

SEQ ID NO: 35 - DNA, 21 bp, synthetic construct
  misc_feature 1..21: note = SNORD115 artificial/synthetic sequence
  source 1..21: mol_type = other DNA
ttctgatttggtgagaaataa

SEQ ID NO: 36 - DNA, 21 bp, synthetic construct
  misc_feature 1..21: note = SNORD115 artificial/synthetic sequence
  source 1..21: mol_type = other DNA
tctgatttggtgagaaataat

SEQ ID NO: 37 - DNA, 21 bp, synthetic construct
  misc_feature 1..21: note = SNORD115 artificial/synthetic sequence
  source 1..21: mol_type = other DNA
ctgtatcagtgcacctataaa

SEQ ID NO: 38 - DNA, 21 bp, synthetic construct
  misc_feature 1..21: note = SNORD115 artificial/synthetic sequence
  source 1..21: mol_type = other DNA
tgtatcagtgcacctataaat

SEQ ID NO: 39 - DNA, 21 bp, synthetic construct
  misc_feature 1..21: note = SNORD115 artificial/synthetic sequence
  source 1..21: mol_type = other DNA
tttgcttcagtgacctttaat

SEQ ID NO: 40 - DNA, 21 bp, synthetic construct
  misc_feature 1..21: note = SNORD115 artificial/synthetic sequence
  source 1..21: mol_type = other DNA
ttgcttcagtgacctttaatt

SEQ ID NO: 41 - DNA, 21 bp, synthetic construct
  misc_feature 1..21: note = SNORD115 artificial/synthetic sequence
  source 1..21: mol_type = other DNA
tgcttcagtgacctttaattt

SEQ ID NO: 42 - DNA, 21 bp, synthetic construct
  misc_feature 1..21: note = SNORD115 artificial/synthetic sequence
  source 1..21: mol_type = other DNA
agagatgtgccattctattat

SEQ ID NO: 43 - DNA, 21 bp, synthetic construct
  misc_feature 1..21: note = SNORD115 artificial/synthetic sequence
  source 1..21: mol_type = other DNA
gagatgtgccattctattata

SEQ ID NO: 44 - DNA, 21 bp, synthetic construct
  misc_feature 1..21: note = SNORD115 artificial/synthetic sequence
  source 1..21: mol_type = other DNA
agatgtgccattctattataa

SEQ ID NO: 45 - DNA, 21 bp, synthetic construct
  misc_feature 1..21: note = SNORD115 artificial/synthetic sequence
  source 1..21: mol_type = other DNA
gatgtgccattctattataaa

SEQ ID NO: 46 - DNA, 21 bp, synthetic construct
  misc_feature 1..21: note = SNORD115 artificial/synthetic sequence
  source 1..21: mol_type = other DNA
tcctttaccgacgtgtatatt

SEQ ID NO: 47 - DNA, 21 bp, synthetic construct
  misc_feature 1..21: note = SNORD115 artificial/synthetic sequence
  source 1..21: mol_type = other DNA
actgttacctagtccaatatt

SEQ ID NO: 48 - DNA, 21 bp, synthetic construct
  misc_feature 1..21: note = SNORD115 artificial/synthetic sequence
  source 1..21: mol_type = other DNA
ctgttacctagtccaatattt

SEQ ID NO: 49 - DNA, 21 bp, synthetic construct
  misc_feature 1..21: note = SNORD115 artificial/synthetic sequence
  source 1..21: mol_type = other DNA
tgttacctagtccaatattta

SEQ ID NO: 50 - DNA, 21 bp, synthetic construct
  misc_feature 1..21: note = SNORD115 artificial/synthetic sequence
  source 1..21: mol_type = other DNA
gttacctagtccaatatttaa

SEQ ID NO: 51 - DNA, 21 bp, synthetic construct
  misc_feature 1..21: note = SNORD115 artificial/synthetic sequence
  source 1..21: mol_type = other DNA
agtatgtactttgcctataaa

SEQ ID NO: 52 - DNA, 21 bp, synthetic construct
  misc_feature 1..21: note = SNORD115 artificial/synthetic sequence
  source 1..21: mol_type = other DNA
gcacatgctcagaagaaataa

SEQ ID NO: 53 - DNA, 21 bp, synthetic construct
  misc_feature 1..21: note = SNORD115 artificial/synthetic sequence
  source 1..21: mol_type = other DNA
cacatgctcagaagaaataaa

SEQ ID NO: 54 - DNA, 21 bp, synthetic construct
  misc_feature 1..21: note = SNORD115 artificial/synthetic sequence
  source 1..21: mol_type = other DNA
ttgagggcttcagtgtttaaa

SEQ ID NO: 55 - DNA, 21 bp, synthetic construct
  misc_feature 1..21: note = SNORD115 artificial/synthetic sequence
  source 1..21: mol_type = other DNA
tggagatctttaacctttaat

SEQ ID NO: 56 - DNA, 21 bp, synthetic construct
  misc_feature 1..21: note = SNORD115 artificial/synthetic sequence
  source 1..21: mol_type = other DNA
gactcagctttcagcttattt

SEQ ID NO: 57 - DNA, 21 bp, synthetic construct
  misc_feature 1..21: note = SNORD115 artificial/synthetic sequence
  source 1..21: mol_type = other DNA
tttctcttggcgtagtaaatt

SEQ ID NO: 58 - DNA, 21 bp, synthetic construct
  misc_feature 1..21: note = SNORD115 artificial/synthetic sequence
  source 1..21: mol_type = other DNA
gatgacacttattcctaatat

SEQ ID NO: 59 - DNA, 21 bp, synthetic construct
  misc_feature 1..21: note = SNORD115 artificial/synthetic sequence
  source 1..21: mol_type = other DNA
atgactgggttcacaaattta

SEQ ID NO: 60 - DNA, 21 bp, synthetic construct
  misc_feature 1..21: note = SNORD115 artificial/synthetic sequence
  source 1..21: mol_type = other DNA
agaatcatggatctcattata

SEQ ID NO: 61 - DNA, 21 bp, synthetic construct
  misc_feature 1..21: note = SNORD115 artificial/synthetic sequence
  source 1..21: mol_type = other DNA
ggtctagagccttagtaatat

SEQ ID NO: 62 - DNA, 21 bp, synthetic construct
  misc_feature 1..21: note = SNORD115 artificial/synthetic sequence
  source 1..21: mol_type = other DNA
aggcatgctgcagtgaattta

SEQ ID NO: 63 - DNA, 21 bp, synthetic construct
  misc_feature 1..21: note = SNORD115 artificial/synthetic sequence
  source 1..21: mol_type = other DNA
ggcatgctgcagtgaatttaa

SEQ ID NO: 64 - DNA, 21 bp, synthetic construct
  misc_feature 1..21: note = SNORD115 artificial/synthetic sequence
  source 1..21: mol_type = other DNA
tatcttcaggagacgtaataa

SEQ ID NO: 65 - DNA, 21 bp, synthetic construct
  misc_feature 1..21: note = SNORD115 artificial/synthetic sequence
  source 1..21: mol_type = other DNA
atcttcaggagacgtaataat

SEQ ID NO: 66 - DNA, 21 bp, synthetic construct
  misc_feature 1..21: note = SNORD115 artificial/synthetic sequence
  source 1..21: mol_type = other DNA
gagagaatatcttggtttatt

SEQ ID NO: 67 - DNA, 21 bp, synthetic construct
  misc_feature 1..21: note = SNORD115 artificial/synthetic sequence
  source 1..21: mol_type = other DNA
tgtgtcattacacggattata

SEQ ID NO: 68 - DNA, 21 bp, synthetic construct
  misc_feature 1..21: note = SNORD115 artificial/synthetic sequence
  source 1..21: mol_type = other DNA
gtgtcattacacggattatat

SEQ ID NO: 69 - DNA, 21 bp, synthetic construct
  misc_feature 1..21: note = SNORD115 artificial/synthetic sequence
  source 1..21: mol_type = other DNA
tgtcattacacggattatata

SEQ ID NO: 70 - DNA, 21 bp, synthetic construct
  misc_feature 1..21: note = SNORD115 artificial/synthetic sequence
  source 1..21: mol_type = other DNA
gtcattacacggattatatat

SEQ ID NO: 71 - DNA, 21 bp, synthetic construct
  misc_feature 1..21: note = SNORD115 artificial/synthetic sequence
  source 1..21: mol_type = other DNA
gagttctgtctttcgtaatta

SEQ ID NO: 72 - DNA, 21 bp, synthetic construct
  misc_feature 1..21: note = SNORD115 artificial/synthetic sequence
  source 1..21: mol_type = other DNA
agttctgtctttcgtaattaa

SEQ ID NO: 73 - DNA, 21 bp, synthetic construct
  misc_feature 1..21: note = SNORD115 artificial/synthetic sequence
  source 1..21: mol_type = other DNA
gttctgtctttcgtaattaaa

SEQ ID NO: 74 - DNA, 21 bp, synthetic construct
  misc_feature 1..21: note = SNORD115 artificial/synthetic sequence
  source 1..21: mol_type = other DNA
caagtggaccatttgaatatt

SEQ ID NO: 75 - DNA, 21 bp, synthetic construct
  misc_feature 1..21: note = SNORD115 artificial/synthetic sequence
  source 1..21: mol_type = other DNA
agtggaccatttgaatattaa

SEQ ID NO: 76 - DNA, 21 bp, synthetic construct
  misc_feature 1..21: note = SNORD115 artificial/synthetic sequence
  source 1..21: mol_type = other DNA
gtggaccatttgaatattaaa

SEQ ID NO: 77 - DNA, 21 bp, synthetic construct
  misc_feature 1..21: note = SNORD115 artificial/synthetic sequence
  source 1..21: mol_type = other DNA
cctgaacgtactttgatttaa

SEQ ID NO: 78 - DNA, 21 bp, synthetic construct
  misc_feature 1..21: note = SNORD115 artificial/synthetic sequence
  source 1..21: mol_type = other DNA
ctgaacgtactttgatttaaa

SEQ ID NO: 79 - DNA, 21 bp, synthetic construct
  misc_feature 1..21: note = SNORD115 artificial/synthetic sequence
  source 1..21: mol_type = other DNA
ggtgtcattagattcattata

SEQ ID NO: 80 - DNA, 21 bp, synthetic construct
  misc_feature 1..21: note = SNORD115 artificial/synthetic sequence
  source 1..21: mol_type = other DNA
cttaagtaacgggtcatattt

SEQ ID NO: 81 - DNA, 21 bp, synthetic construct
  misc_feature 1..21: note = SNORD115 artificial/synthetic sequence
  source 1..21: mol_type = other DNA
atctcagtcttgtagaaatta

SEQ ID NO: 82 - DNA, 21 bp, synthetic construct
  misc_feature 1..21: note = SNORD115 artificial/synthetic sequence
  source 1..21: mol_type = other DNA
ttaggagtcatgcacatataa

SEQ ID NO: 83 - DNA, 21 bp, synthetic construct
  misc_feature 1..21: note = SNORD115 artificial/synthetic sequence
  source 1..21: mol_type = other DNA
ccatctctggtaagcatattt

SEQ ID NO: 84 - DNA, 21 bp, synthetic construct
  misc_feature 1..21: note = SNORD115 artificial/synthetic sequence
  source 1..21: mol_type = other DNA
gtttcctacaattcctaattt

SEQ ID NO: 85 - DNA, 21 bp, synthetic construct
  misc_feature 1..21: note = SNORD115 artificial/synthetic sequence
  source 1..21: mol_type = other DNA
gtgaagactgagctctataat

SEQ ID NO: 86 - DNA, 21 bp, synthetic construct
  misc_feature 1..21: note = SNORD115 artificial/synthetic sequence
  source 1..21: mol_type = other DNA
tgaagactgagctctataata

SEQ ID NO: 87 - DNA, 21 bp, synthetic construct
  misc_feature 1..21: note = SNORD115 artificial/synthetic sequence
  source 1..21: mol_type = other DNA
gaagactgagctctataataa

SEQ ID NO: 88 - DNA, 21 bp, synthetic construct
  misc_feature 1..21: note = SNORD115 artificial/synthetic sequence
  source 1..21: mol_type = other DNA
agactgagctctataataatt

SEQ ID NO: 89 - DNA, 21 bp, synthetic construct
  misc_feature 1..21: note = SNORD115 artificial/synthetic sequence
  source 1..21: mol_type = other DNA
gactgagctctataataatta

SEQ ID NO: 90 - DNA, 21 bp, synthetic construct
  misc_feature 1..21: note = SNORD115 artificial/synthetic sequence
  source 1..21: mol_type = other DNA
acagttatcgtggtctaaata

SEQ ID NO: 91 - DNA, 21 bp, synthetic construct
  misc_feature 1..21: note = SNORD115 artificial/synthetic sequence
  source 1..21: mol_type = other DNA
cagttatcgtggtctaaatat

SEQ ID NO: 92 - DNA, 21 bp, synthetic construct
  misc_feature 1..21: note = SNORD115 artificial/synthetic sequence
  source 1..21: mol_type = other DNA
agttatcgtggtctaaatata

SEQ ID NO: 93 - DNA, 21 bp, synthetic construct
  misc_feature 1..21: note = SNORD115 artificial/synthetic sequence
  source 1..21: mol_type = other DNA
tgcccatgaaacttgaaatta

SEQ ID NO: 94 - DNA, 21 bp, synthetic construct
  misc_feature 1..21: note = SNORD115 artificial/synthetic sequence
  source 1..21: mol_type = other DNA
gaataatgccaatggtaaata

SEQ ID NO: 95 - DNA, 21 bp, synthetic construct
  misc_feature 1..21: note = SNORD115 artificial/synthetic sequence
  source 1..21: mol_type = other DNA
gacctttatggacacttatat

SEQ ID NO: 96 - DNA, 21 bp, synthetic construct
  misc_feature 1..21: note = SNORD115 artificial/synthetic sequence
  source 1..21: mol_type = other DNA
ctacaagagggaaactttatt

SEQ ID NO: 97 - DNA, 21 bp, synthetic construct
  misc_feature 1..21: note = SNORD115 artificial/synthetic sequence
  source 1..21: mol_type = other DNA
tacaagagggaaactttattt

SEQ ID NO: 98 - DNA, 21 bp, synthetic construct
  misc_feature 1..21: note = SNORD115 artificial/synthetic sequence
  source 1..21: mol_type = other DNA
gatgctcatttggtctaaata

SEQ ID NO: 99 - DNA, 21 bp, synthetic construct
  misc_feature 1..21: note = SNORD115 artificial/synthetic sequence
  source 1..21: mol_type = other DNA
atgctcatttggtctaaatat

SEQ ID NO: 100 - DNA, 21 bp, synthetic construct
  misc_feature 1..21: note = SNORD115 artificial/synthetic sequence
  source 1..21: mol_type = other DNA
tgctcatttggtctaaatata

SEQ ID NO: 101 - DNA, 21 bp, synthetic construct
  misc_feature 1..21: note = SNORD115 artificial/synthetic sequence
  source 1..21: mol_type = other DNA
catatcccatcattcattaaa

SEQ ID NO: 102 - DNA, 21 bp, synthetic construct
  misc_feature 1..21: note = SNORD115 artificial/synthetic sequence
  source 1..21: mol_type = other DNA
ggctccagtatgatcattata

SEQ ID NO: 103 - DNA, 21 bp, synthetic construct
  misc_feature 1..21: note = SNORD115 artificial/synthetic sequence
  source 1..21: mol_type = other DNA
gctccagtatgatcattatat

SEQ ID NO: 104 - DNA, 21 bp, synthetic construct
  misc_feature 1..21: note = SNORD115 artificial/synthetic sequence
  source 1..21: mol_type = other DNA
gatcaattgaaggtcttattt

SEQ ID NO: 105 - DNA, 21 bp, synthetic construct
  misc_feature 1..21: note = SNORD115 artificial/synthetic sequence
  source 1..21: mol_type = other DNA
aggtcttatttctgcattaaa

SEQ ID NO: 106 - DNA, 21 bp, synthetic construct
  misc_feature 1..21: note = SNORD115 artificial/synthetic sequence
  source 1..21: mol_type = other DNA
cctgtatagttagtctttatt

SEQ ID NO: 107 - DNA, 21 bp, synthetic construct
  misc_feature 1..21: note = SNORD115 artificial/synthetic sequence
  source 1..21: mol_type = other DNA
catacatacaggaggtaatat

SEQ ID NO: 108 - DNA, 21 bp, synthetic construct
  misc_feature 1..21: note = SNORD115 artificial/synthetic sequence
  source 1..21: mol_type = other DNA
atacatacaggaggtaatatt

SEQ ID NO: 109 - DNA, 21 bp, synthetic construct
  misc_feature 1..21: note = SNORD115 artificial/synthetic sequence
  source 1..21: mol_type = other DNA
tacatacaggaggtaatattt

SEQ ID NO: 110 - DNA, 21 bp, synthetic construct
  misc_feature 1..21: note = SNORD115 artificial/synthetic sequence
  source 1..21: mol_type = other DNA
gaactttcaaccaggatttaa

SEQ ID NO: 111 - DNA, 21 bp, synthetic construct
  misc_feature 1..21: note = SNORD115 artificial/synthetic sequence
  source 1..21: mol_type = other DNA
tggctgaaacctcccatattt

SEQ ID NO: 112 - DNA, 21 bp, synthetic construct
  misc_feature 1..21: note = SNORD115 artificial/synthetic sequence
  source 1..21: mol_type = other DNA
agcaaaggcataacatattaa

SEQ ID NO: 113 - DNA, 21 bp, synthetic construct
  misc_feature 1..21: note = SNORD115 artificial/synthetic sequence
  source 1..21: mol_type = other DNA
gcaaaggcataacatattaaa

SEQ ID NO: 114 - DNA, 21 bp, synthetic construct
  misc_feature 1..21: note = SNORD115 artificial/synthetic sequence
  source 1..21: mol_type = other DNA
gactacagatggtagattaat

SEQ ID NO: 115 - DNA, 21 bp, synthetic construct
  misc_feature 1..21: note = SNORD115 artificial/synthetic sequence
  source 1..21: mol_type = other DNA
actacagatggtagattaata

SEQ ID NO: 116 - DNA, 21 bp, synthetic construct
  misc_feature 1..21: note = SNORD115 artificial/synthetic sequence
  source 1..21: mol_type = other DNA
ctacagatggtagattaatat

SEQ ID NO: 117 - DNA, 21 bp, synthetic construct
  misc_feature 1..21: note = SNORD115 artificial/synthetic sequence
  source 1..21: mol_type = other DNA
ttggcttatagcatcaataaa

SEQ ID NO: 118 - DNA, 21 bp, synthetic construct
  misc_feature 1..21: note = SNORD115 artificial/synthetic sequence
  source 1..21: mol_type = other DNA
tggcttatagcatcaataaat

SEQ ID NO: 119 - DNA, 21 bp, synthetic construct
  misc_feature 1..21: note = SNORD115 artificial/synthetic sequence
  source 1..21: mol_type = other DNA
tttgggtgagttaagatatat

SEQ ID NO: 120 - DNA, 21 bp, synthetic construct
  misc_feature 1..21: note = SNORD115 artificial/synthetic sequence
  source 1..21: mol_type = other DNA
gagaagaagaatgacataata

SEQ ID NO: 121 - DNA, 21 bp, synthetic construct
  misc_feature 1..21: note = SNORD115 artificial/synthetic sequence
  source 1..21: mol_type = other DNA
tatctcatttcacccatatat

SEQ ID NO: 122 - DNA, 21 bp, synthetic construct
  misc_feature 1..21: note = SNORD115 artificial/synthetic sequence
  source 1..21: mol_type = other DNA
atctcatttcacccatatata

SEQ ID NO: 123 - DNA, 21 bp, synthetic construct
  misc_feature 1..21: note = SNORD115 artificial/synthetic sequence
  source 1..21: mol_type = other DNA
tctcatttcacccatatatat

SEQ ID NO: 124 - DNA, 21 bp, synthetic construct
  misc_feature 1..21: note = SNORD115 artificial/synthetic sequence
  source 1..21: mol_type = other DNA
gggtatggatgggacaaatat

SEQ ID NO: 125 - DNA, 21 bp, synthetic construct
  misc_feature 1..21: note = SNORD115 artificial/synthetic sequence
  source 1..21: mol_type = other DNA
ccctgaaataacagcattaat

SEQ ID NO: 126 - DNA, 21 bp, synthetic construct
  misc_feature 1..21: note = SNORD115 artificial/synthetic sequence
  source 1..21: mol_type = other DNA
atttgaccattcccaattata

SEQ ID NO: 127 - DNA, 21 bp, synthetic construct
  misc_feature 1..21: note = SNORD115 artificial/synthetic sequence
  source 1..21: mol_type = other DNA
tggatgtaagaacactattaa

SEQ ID NO: 128 - DNA, 21 bp, synthetic construct
  misc_feature 1..21: note = SNORD115 artificial/synthetic sequence
  source 1..21: mol_type = other DNA
atagcagaatgcacattatat

SEQ ID NO: 129 - DNA, 21 bp, synthetic construct
  misc_feature 1..21: note = SNORD115 artificial/synthetic sequence
  source 1..21: mol_type = other DNA
tagcagaatgcacattatatt

SEQ ID NO: 130 - DNA, 21 bp, synthetic construct
  misc_feature 1..21: note = SNORD115 artificial/synthetic sequence
  source 1..21: mol_type = other DNA
agtgctcataaagcattattt

SEQ ID NO: 131 - DNA, 21 bp, synthetic construct
  misc_feature 1..21: note = SNORD115 artificial/synthetic sequence
  source 1..21: mol_type = other DNA
agagtgcctatgtagttaata

SEQ ID NO: 132 - DNA, 21 bp, synthetic construct
  misc_feature 1..21: note = SNORD115 artificial/synthetic sequence
  source 1..21: mol_type = other DNA
gagtgcctatgtagttaatat

SEQ ID NO: 133 - DNA, 21 bp, synthetic construct
  misc_feature 1..21: note = SNORD115 artificial/synthetic sequence
  source 1..21: mol_type = other DNA
agtgcctatgtagttaatatt

SEQ ID NO: 134 - DNA, 21 bp, synthetic construct
  misc_feature 1..21: note = SNORD115 artificial/synthetic sequence
  source 1..21: mol_type = other DNA
gtgcctatgtagttaatattt

SEQ ID NO: 135 - DNA, 21 bp, synthetic construct
  misc_feature 1..21: note = SNORD115 artificial/synthetic sequence
  source 1..21: mol_type = other DNA
accacaggcttctcaattatt

SEQ ID NO: 136 - DNA, 21 bp, synthetic construct
  misc_feature 1..21: note = SNORD115 artificial/synthetic sequence
  source 1..21: mol_type = other DNA
ccacaggcttctcaattatta

SEQ ID NO: 137 - DNA, 21 bp, synthetic construct
  misc_feature 1..21: note = SNORD115 artificial/synthetic sequence
  source 1..21: mol_type = other DNA
cacaggcttctcaattattaa

SEQ ID NO: 138 - DNA, 21 bp, synthetic construct
  misc_feature 1..21: note = SNORD115 artificial/synthetic sequence
  source 1..21: mol_type = other DNA
acaggcttctcaattattaat

SEQ ID NO: 139 - DNA, 21 bp, synthetic construct
  misc_feature 1..21: note = SNORD115 artificial/synthetic sequence
  source 1..21: mol_type = other DNA
caggcttctcaattattaatt

SEQ ID NO: 140 - DNA, 21 bp, synthetic construct
  misc_feature 1..21: note = SNORD115 artificial/synthetic sequence
  source 1..21: mol_type = other DNA
tcctctagaactgcctttaaa

SEQ ID NO: 141 - DNA, 21 bp, synthetic construct
  misc_feature 1..21: note = SNORD115 artificial/synthetic sequence
  source 1..21: mol_type = other DNA
cctctagaactgcctttaaat

SEQ ID NO: 142 - DNA, 21 bp, synthetic construct
  misc_feature 1..21: note = SNORD115 artificial/synthetic sequence
  source 1..21: mol_type = other DNA
actgcctttaaatgcatatta

SEQ ID NO: 143 - DNA, 21 bp, synthetic construct
  misc_feature 1..21: note = SNORD115 artificial/synthetic sequence
  source 1..21: mol_type = other DNA
tcaggagttaataggatattt

SEQ ID NO: 144 - DNA, 21 bp, synthetic construct
  misc_feature 1..21: note = SNORD115 artificial/synthetic sequence
  source 1..21: mol_type = other DNA
acaacacagatcttctattat

SEQ ID NO: 145 - DNA, 21 bp, synthetic construct
  misc_feature 1..21: note = SNORD115 artificial/synthetic sequence
  source 1..21: mol_type = other DNA
acggcacaccaggagattata

SEQ ID NO: 146 - DNA, 21 bp, synthetic construct
  misc_feature 1..21: note = SNORD115 artificial/synthetic sequence
  source 1..21: mol_type = other DNA
cggcacaccaggagattatat

SEQ ID NO: 147 - DNA, 21 bp, synthetic construct
  misc_feature 1..21: note = SNORD115 artificial/synthetic sequence
  source 1..21: mol_type = other DNA
agacaggctgcctccttaaat

SEQ ID NO: 148 - DNA, 21 bp, synthetic construct
  misc_feature 1..21: note = SNORD115 artificial/synthetic sequence
  source 1..21: mol_type = other DNA
catttgccgttctgcaatatt

SEQ ID NO: 149 - DNA, 21 bp, synthetic construct
  misc_feature 1..21: note = SNORD115 artificial/synthetic sequence
  source 1..21: mol_type = other DNA
atttgccgttctgcaatattt

SEQ ID NO: 150 - DNA, 21 bp, synthetic construct
  misc_feature 1..21: note = SNORD115 artificial/synthetic sequence
  source 1..21: mol_type = other DNA
gcttcagatgatcgctaataa

SEQ ID NO: 151 - DNA, 21 bp, synthetic construct
  misc_feature 1..21: note = SNORD115 artificial/synthetic sequence
  source 1..21: mol_type = other DNA
tgagtggctaactagaataaa

SEQ ID NO: 152 - DNA, 21 bp, synthetic construct
  misc_feature 1..21: note = SNORD115 artificial/synthetic sequence
  source 1..21: mol_type = other DNA
agcatagagaagaccttaaat

SEQ ID NO: 153 - DNA, 21 bp, synthetic construct
  misc_feature 1..21: note = SNORD115 artificial/synthetic sequence
  source 1..21: mol_type = other DNA
gtaactccaagacacataatt

SEQ ID NO: 154 - DNA, 21 bp, synthetic construct
  misc_feature 1..21: note = SNORD115 artificial/synthetic sequence
  source 1..21: mol_type = other DNA
gccaaactaagcttcataatt

SEQ ID NO: 155 - DNA, 21 bp, synthetic construct
  misc_feature 1..21: note = SNORD115 artificial/synthetic sequence
  source 1..21: mol_type = other DNA
tgggctaaattctccaattaa

SEQ ID NO: 156 - DNA, 21 bp, synthetic construct
  misc_feature 1..21: note = SNORD115 artificial/synthetic sequence
  source 1..21: mol_type = other DNA
gggctaaattctccaattaaa

SEQ ID NO: 157 - DNA, 21 bp, synthetic construct
  misc_feature 1..21: note = SNORD115 artificial/synthetic sequence
  source 1..21: mol_type = other DNA
acacacataggctccaaataa

SEQ ID NO: 158 - DNA, 21 bp, synthetic construct
  misc_feature 1..21: note = SNORD115 artificial/synthetic sequence
  source 1..21: mol_type = other DNA
cacacataggctccaaataaa

SEQ ID NO: 159 - DNA, 21 bp, synthetic construct
  misc_feature 1..21: note = SNORD115 artificial/synthetic sequence
  source 1..21: mol_type = other DNA
tctgatgaaacagactttaaa

SEQ ID NO: 160 - DNA, 21 bp, synthetic construct
  misc_feature 1..21: note = SNORD115 artificial/synthetic sequence
  source 1..21: mol_type = other DNA
agagctaactatcctaaatat

SEQ ID NO: 161 - DNA, 21 bp, synthetic construct
  misc_feature 1..21: note = SNORD115 artificial/synthetic sequence
  source 1..21: mol_type = other DNA
gagctaactatcctaaatata

SEQ ID NO: 162 - DNA, 21 bp, synthetic construct
  misc_feature 1..21: note = SNORD115 artificial/synthetic sequence
  source 1..21: mol_type = other DNA
cttagactcccacacaataat

SEQ ID NO: 163 - DNA, 21 bp, synthetic construct
  misc_feature 1..21: note = SNORD115 artificial/synthetic sequence
  source 1..21: mol_type = other DNA
ttagactcccacacaataata

SEQ ID NO: 164 - DNA, 21 bp, synthetic construct
  misc_feature 1..21: note = SNORD115 artificial/synthetic sequence
  source 1..21: mol_type = other DNA
tagactcccacacaataataa

SEQ ID NO: 165 - DNA, 21 bp, synthetic construct
  misc_feature 1..21: note = SNORD115 artificial/synthetic sequence
  source 1..21: mol_type = other DNA
agactcccacacaataataat

SEQ ID NO: 166 - DNA, 21 bp, synthetic construct
  misc_feature 1..21: note = SNORD115 artificial/synthetic sequence
  source 1..21: mol_type = other DNA
atgaaatgaaggcagaaataa

SEQ ID NO: 167 - DNA, 21 bp, synthetic construct
  misc_feature 1..21: note = SNORD115 artificial/synthetic sequence
  source 1..21: mol_type = other DNA
tgaaatgaaggcagaaataaa

SEQ ID NO: 168 - DNA, 21 bp, synthetic construct
  misc_feature 1..21: note = SNORD115 artificial/synthetic sequence
  source 1..21: mol_type = other DNA
cagaatctctgggacatattt

SEQ ID NO: 169 - DNA, 21 bp, synthetic construct
  misc_feature 1..21: note = SNORD115 artificial/synthetic sequence
  source 1..21: mol_type = other DNA
agaatctctgggacatattta

SEQ ID NO: 170 - DNA, 21 bp, synthetic construct
  misc_feature 1..21: note = SNORD115 artificial/synthetic sequence
  source 1..21: mol_type = other DNA
gaatctctgggacatatttaa

SEQ ID NO: 171 - DNA, 21 bp, synthetic construct
  misc_feature 1..21: note = SNORD115 artificial/synthetic sequence
  source 1..21: mol_type = other DNA
gcagtgtgtagagagaaattt

SEQ ID NO: 172 - DNA, 21 bp, synthetic construct
  misc_feature 1..21: note = SNORD115 artificial/synthetic sequence
  source 1..21: mol_type = other DNA
cagtgtgtagagagaaattta

SEQ ID NO: 173 - DNA, 21 bp, synthetic construct
  misc_feature 1..21: note = SNORD115 artificial/synthetic sequence
  source 1..21: mol_type = other DNA
agtgtgtagagagaaatttat

SEQ ID NO: 174 - DNA, 21 bp, synthetic construct
  misc_feature 1..21: note = SNORD115 artificial/synthetic sequence
  source 1..21: mol_type = other DNA
gtgtgtagagagaaatttata

SEQ ID NO: 175 - DNA, 21 bp, synthetic construct
  misc_feature 1..21: note = SNORD115 artificial/synthetic sequence
  source 1..21: mol_type = other DNA
acaccctaacatcacaattaa

SEQ ID NO: 176 - DNA, 21 bp, synthetic construct
  misc_feature 1..21: note = SNORD115 artificial/synthetic sequence
  source 1..21: mol_type = other DNA
caccctaacatcacaattaaa

SEQ ID NO: 177 - DNA, 21 bp, synthetic construct
  misc_feature 1..21: note = SNORD115 artificial/synthetic sequence
  source 1..21: mol_type = other DNA
ctagcagaaggcaggaaataa

SEQ ID NO: 178 - DNA, 21 bp, synthetic construct
  misc_feature 1..21: note = SNORD115 artificial/synthetic sequence
  source 1..21: mol_type = other DNA
gaccactagcaagactaataa

SEQ ID NO: 179 - DNA, 21 bp, synthetic construct
  misc_feature 1..21: note = SNORD115 artificial/synthetic sequence
  source 1..21: mol_type = other DNA
accactagcaagactaataaa

SEQ ID NO: 180 - DNA, 21 bp, synthetic construct
  misc_feature 1..21: note = SNORD115 artificial/synthetic sequence
  source 1..21: mol_type = other DNA
gaatccaatagatgcaataaa

SEQ ID NO: 181 - DNA, 21 bp, synthetic construct
  misc_feature 1..21: note = SNORD115 artificial/synthetic sequence
  source 1..21: mol_type = other DNA
accatcagagaatactataaa

SEQ ID NO: 182 - DNA, 21 bp, synthetic construct
  misc_feature 1..21: note = SNORD115 artificial/synthetic sequence
  source 1..21: mol_type = other DNA
ctctgaaattgaggcaataat

SEQ ID NO: 183 - DNA, 21 bp, synthetic construct
  misc_feature 1..21: note = SNORD115 artificial/synthetic sequence
  source 1..21: mol_type = other DNA
tctgaaattgaggcaataatt

SEQ ID NO: 184 - DNA, 21 bp, synthetic construct
  misc_feature 1..21: note = SNORD115 artificial/synthetic sequence
  source 1..21: mol_type = other DNA
ctgaaattgaggcaataatta

SEQ ID NO: 185 - DNA, 21 bp, synthetic construct
  misc_feature 1..21: note = SNORD115 artificial/synthetic sequence
  source 1..21: mol_type = other DNA
tcaggcaggagaaagaaataa

SEQ ID NO: 186 - DNA, 21 bp, synthetic construct
  misc_feature 1..21: note = SNORD115 artificial/synthetic sequence
  source 1..21: mol_type = other DNA
caggcaggagaaagaaataaa

SEQ ID NO: 187 - DNA, 21 bp, synthetic construct
  misc_feature 1..21: note = SNORD115 artificial/synthetic sequence
  source 1..21: mol_type = other DNA
cagatgacatgattgtatatt

SEQ ID NO: 188 - DNA, 21 bp, synthetic construct
  misc_feature 1..21: note = SNORD115 artificial/synthetic sequence
  source 1..21: mol_type = other DNA
accactgctcaacgaaataaa

SEQ ID NO: 189 - DNA, 21 bp, synthetic construct
  misc_feature 1..21: note = SNORD115 artificial/synthetic sequence
  source 1..21: mol_type = other DNA
ccatactgcccaaggtaattt

SEQ ID NO: 190 - DNA, 21 bp, synthetic construct
  misc_feature 1..21: note = SNORD115 artificial/synthetic sequence
  source 1..21: mol_type = other DNA
catactgcccaaggtaattta

SEQ ID NO: 191 - DNA, 21 bp, synthetic construct
  misc_feature 1..21: note = SNORD115 artificial/synthetic sequence
  source 1..21: mol_type = other DNA
atactgcccaaggtaatttat

SEQ ID NO: 192 - DNA, 21 bp, synthetic construct
  misc_feature 1..21: note = SNORD115 artificial/synthetic sequence
  source 1..21: mol_type = other DNA
tactgcccaaggtaatttata

SEQ ID NO: 193 - DNA, 21 bp, synthetic construct
  misc_feature 1..21: note = SNORD115 artificial/synthetic sequence
  source 1..21: mol_type = other DNA
ggaaaggattccctatttaat

SEQ ID NO: 194 - DNA, 21 bp, synthetic construct
  misc_feature 1..21: note = SNORD115 artificial/synthetic sequence
  source 1..21: mol_type = other DNA
gaaaggattccctatttaata

SEQ ID NO: 195 - DNA, 21 bp, synthetic construct
  misc_feature 1..21: note = SNORD115 artificial/synthetic sequence
  source 1..21: mol_type = other DNA
gttagaatggcaatcattaaa

SEQ ID NO: 196 - DNA, 21 bp, synthetic construct
  misc_feature 1..21: note = SNORD115 artificial/synthetic sequence
  source 1..21: mol_type = other DNA
gtacatacccaaaggattata

SEQ ID NO: 197 - DNA, 21 bp, synthetic construct
  misc_feature 1..21: note = SNORD115 artificial/synthetic sequence
  source 1..21: mol_type = other DNA
tacatacccaaaggattatat

SEQ ID NO: 198 - DNA, 21 bp, synthetic construct
  misc_feature 1..21: note = SNORD115 artificial/synthetic sequence
  source 1..21: mol_type = other DNA
acatacccaaaggattatata

SEQ ID NO: 199 - DNA, 21 bp, synthetic construct
  misc_feature 1..21: note = SNORD115 artificial/synthetic sequence
  source 1..21: mol_type = other DNA
catacccaaaggattatatat

SEQ ID NO: 200 - DNA, 21 bp, synthetic construct
  misc_feature 1..21: note = SNORD115 artificial/synthetic sequence
  source 1..21: mol_type = other DNA
cacgaagtaggacagtataat

SEQ ID NO: 201 - DNA, 21 bp, synthetic construct
  misc_feature 1..21: note = SNORD115 artificial/synthetic sequence
  source 1..21: mol_type = other DNA
acgaagtaggacagtataatt

SEQ ID NO: 202 - DNA, 21 bp, synthetic construct
  misc_feature 1..21: note = SNORD115 artificial/synthetic sequence
  source 1..21: mol_type = other DNA
cgaagtaggacagtataattt

SEQ ID NO: 203 - DNA, 21 bp, synthetic construct
  misc_feature 1..21: note = SNORD115 artificial/synthetic sequence
  source 1..21: mol_type = other DNA
accaccatgcctggctaattt

SEQ ID NO: 204 - DNA, 21 bp, synthetic construct
  misc_feature 1..21: note = SNORD115 artificial/synthetic sequence
  source 1..21: mol_type = other DNA
ttaagaacaagacccaaatat

SEQ ID NO: 205 - DNA, 21 bp, synthetic construct
  misc_feature 1..21: note = SNORD115 artificial/synthetic sequence
  source 1..21: mol_type = other DNA
taagaacaagacccaaatata

SEQ ID NO: 206 - DNA, 21 bp, synthetic construct
  misc_feature 1..21: note = SNORD115 artificial/synthetic sequence
  source 1..21: mol_type = other DNA
ctatgagaaatgcactttaaa

SEQ ID NO: 207 - DNA, 21 bp, synthetic construct
  misc_feature 1..21: note = SNORD115 artificial/synthetic sequence
  source 1..21: mol_type = other DNA
atatcccatgctaacattaat

SEQ ID NO: 208 - DNA, 21 bp, synthetic construct
  misc_feature 1..21: note = SNORD115 artificial/synthetic sequence
  source 1..21: mol_type = other DNA
gactttagaacaaggaatatt

SEQ ID NO: 209 - DNA, 21 bp, synthetic construct
  misc_feature 1..21: note = SNORD115 artificial/synthetic sequence
  source 1..21: mol_type = other DNA
atgcacctcaacaaatttaaa

SEQ ID NO: 210 - DNA, 21 bp, synthetic construct
  misc_feature 1..21: note = SNORD115 artificial/synthetic sequence
  source 1..21: mol_type = other DNA
cacagaagtctcaagataaat

SEQ ID NO: 211 - DNA, 21 bp, synthetic construct
  misc_feature 1..21: note = SNORD115 artificial/synthetic sequence
  source 1..21: mol_type = other DNA
acagaagtctcaagataaatt

SEQ ID NO: 212 - DNA, 21 bp, synthetic construct
  misc_feature 1..21: note = SNORD115 artificial/synthetic sequence
  source 1..21: mol_type = other DNA
cagaagtctcaagataaattt

SEQ ID NO: 213 - DNA, 21 bp, synthetic construct
  misc_feature 1..21: note = SNORD115 artificial/synthetic sequence
  source 1..21: mol_type = other DNA
gcagtgcctactgggaaattt

SEQ ID NO: 214 - DNA, 21 bp, synthetic construct
  misc_feature 1..21: note = SNORD115 artificial/synthetic sequence
  source 1..21: mol_type = other DNA
cagtgcctactgggaaattta

SEQ ID NO: 215 - DNA, 21 bp, synthetic construct
  misc_feature 1..21: note = SNORD115 artificial/synthetic sequence
  source 1..21: mol_type = other DNA
agtgcctactgggaaatttat

SEQ ID NO: 216 - DNA, 21 bp, synthetic construct
  misc_feature 1..21: note = SNORD115 artificial/synthetic sequence
  source 1..21: mol_type = other DNA
gtgcctactgggaaatttata

SEQ ID NO: 217 - DNA, 21 bp, synthetic construct
  misc_feature 1..21: note = SNORD115 artificial/synthetic sequence
  source 1..21: mol_type = other DNA
tgctgcacccagggcaattta

SEQ ID NO: 218 - DNA, 21 bp, synthetic construct
  misc_feature 1..21: note = SNORD115 artificial/synthetic sequence
  source 1..21: mol_type = other DNA
gctgcacccagggcaatttat

SEQ ID NO: 219 - DNA, 21 bp, synthetic construct
  misc_feature 1..21: note = SNORD115 artificial/synthetic sequence
  source 1..21: mol_type = other DNA
ctgcacccagggcaatttata

SEQ ID NO: 220 - DNA, 21 bp, synthetic construct
  misc_feature 1..21: note = SNORD115 artificial/synthetic sequence
  source 1..21: mol_type = other DNA
ctctgataagcatgctaatat

SEQ ID NO: 221 - DNA, 21 bp, synthetic construct
  misc_feature 1..21: note = SNORD115 artificial/synthetic sequence
  source 1..21: mol_type = other DNA
ggcaatatggagatgttatat

SEQ ID NO: 222 - DNA, 21 bp, synthetic construct
  misc_feature 1..21: note = SNORD115 artificial/synthetic sequence
  source 1..21: mol_type = other DNA
agatgcaatcatcccattaat

SEQ ID NO: 223 - DNA, 21 bp, synthetic construct
  misc_feature 1..21: note = SNORD115 artificial/synthetic sequence
  source 1..21: mol_type = other DNA
attctctcttaaggctattat

SEQ ID NO: 224 - DNA, 21 bp, synthetic construct
  misc_feature 1..21: note = SNORD115 artificial/synthetic sequence
  source 1..21: mol_type = other DNA
tgttcacttgggtacatatat

SEQ ID NO: 225 - DNA, 21 bp, synthetic construct
  misc_feature 1..21: note = SNORD115 artificial/synthetic sequence
  source 1..21: mol_type = other DNA
gttcacttgggtacatatata

SEQ ID NO: 226 - DNA, 21 bp, synthetic construct
  misc_feature 1..21: note = SNORD115 artificial/synthetic sequence
  source 1..21: mol_type = other DNA
catgccagtggacacaaatat

SEQ ID NO: 227 - DNA, 21 bp, synthetic construct
  misc_feature 1..21: note = SNORD115 artificial/synthetic sequence
  source 1..21: mol_type = other DNA
tatggcttcctcatgattaaa

SEQ ID NO: 228 - DNA, 21 bp, synthetic construct
  misc_feature 1..21: note = SNORD115 artificial/synthetic sequence
  source 1..21: mol_type = other DNA
gctaccattcctggctaattt

SEQ ID NO: 229 - DNA, 21 bp, synthetic construct
  misc_feature 1..21: note = SNORD115 artificial/synthetic sequence
  source 1..21: mol_type = other DNA
tgctggcacaagaactttata

SEQ ID NO: 230 - DNA, 21 bp, synthetic construct
  misc_feature 1..21: note = SNORD115 artificial/synthetic sequence
  source 1..21: mol_type = other DNA
ccacataccctaaagaattat

SEQ ID NO: 231 - DNA, 21 bp, synthetic construct
  misc_feature 1..21: note = SNORD115 artificial/synthetic sequence
  source 1..21: mol_type = other DNA
cacataccctaaagaattatt

SEQ ID NO: 232 - DNA, 21 bp, synthetic construct
  misc_feature 1..21: note = SNORD115 artificial/synthetic sequence
  source 1..21: mol_type = other DNA
cttaccatccgtttcatttaa

SEQ ID NO: 233 - DNA, 21 bp, synthetic construct
  misc_feature 1..21: note = SNORD115 artificial/synthetic sequence
  source 1..21: mol_type = other DNA
ttaccatccgtttcatttaaa

SEQ ID NO: 234 - DNA, 21 bp, synthetic construct
  misc_feature 1..21: note = SNORD115 artificial/synthetic sequence
  source 1..21: mol_type = other DNA
tttaggaccttaaggataaat

SEQ ID NO: 235 - DNA, 21 bp, synthetic construct
  misc_feature 1..21: note = SNORD115 artificial/synthetic sequence
  source 1..21: mol_type = other DNA
ttaggaccttaaggataaatt

SEQ ID NO: 236 - DNA, 21 bp, synthetic construct
  misc_feature 1..21: note = SNORD115 artificial/synthetic sequence
  source 1..21: mol_type = other DNA
ctcaccaactggatgatttaa

SEQ ID NO: 237 - DNA, 21 bp, synthetic construct
  misc_feature 1..21: note = SNORD115 artificial/synthetic sequence
  source 1..21: mol_type = other DNA
tcaccaactggatgatttaaa

SEQ ID NO: 238 - DNA, 21 bp, synthetic construct
  misc_feature 1..21: note = SNORD115 artificial/synthetic sequence
  source 1..21: mol_type = other DNA
caccaactggatgatttaaat

SEQ ID NO: 239 - DNA, 21 bp, synthetic construct
  misc_feature 1..21: note = SNORD115 artificial/synthetic sequence
  source 1..21: mol_type = other DNA
accaactggatgatttaaatt

SEQ ID NO: 240 - DNA, 21 bp, synthetic construct
  misc_feature 1..21: note = SNORD115 artificial/synthetic sequence
  source 1..21: mol_type = other DNA
tcccactgattagtcaatatt

SEQ ID NO: 241 - DNA, 21 bp, synthetic construct
  misc_feature 1..21: note = SNORD115 artificial/synthetic sequence
  source 1..21: mol_type = other DNA
cccactgattagtcaatatta

SEQ ID NO: 242 - DNA, 21 bp, synthetic construct
  misc_feature 1..21: note = SNORD115 artificial/synthetic sequence
  source 1..21: mol_type = other DNA
gatactgatgagaagaaattt

SEQ ID NO: 243 - DNA, 21 bp, synthetic construct
  misc_feature 1..21: note = SNORD115 artificial/synthetic sequence
  source 1..21: mol_type = other DNA
tcctgagtagctgggattata

SEQ ID NO: 244 - DNA, 21 bp, synthetic construct
  misc_feature 1..21: note = SNORD115 artificial/synthetic sequence
  source 1..21: mol_type = other DNA
accaccatgcctggctaattt

SEQ ID NO: 245 - DNA, 21 bp, synthetic construct
  misc_feature 1..21: note = SNORD115 artificial/synthetic sequence
  source 1..21: mol_type = other DNA
gcccaactctagatcttaaat

SEQ ID NO: 246 - DNA, 21 bp, synthetic construct
  misc_feature 1..21: note = SNORD115 artificial/synthetic sequence
  source 1..21: mol_type = other DNA
cccaactctagatcttaaata

SEQ ID NO: 247 - DNA, 21 bp, synthetic construct
  misc_feature 1..21: note = SNORD115 artificial/synthetic sequence
  source 1..21: mol_type = other DNA
catacctggccagagattatt

SEQ ID NO: 248 - DNA, 21 bp, synthetic construct
  misc_feature 1..21: note = SNORD115 artificial/synthetic sequence
  source 1..21: mol_type = other DNA
atacctggccagagattatta

SEQ ID NO: 249 - DNA, 21 bp, synthetic construct
  misc_feature 1..21: note = SNORD115 artificial/synthetic sequence
  source 1..21: mol_type = other DNA
acctggccagagattattaaa

SEQ ID NO: 250 - DNA, 21 bp, synthetic construct
  misc_feature 1..21: note = SNORD115 artificial/synthetic sequence
  source 1..21: mol_type = other DNA
cctggccagagattattaaat

SEQ ID NO: 251 - DNA, 21 bp, synthetic construct
  misc_feature 1..21: note = SNORD115 artificial/synthetic sequence
  source 1..21: mol_type = other DNA
ctggccagagattattaaata

SEQ ID NO: 252 - DNA, 21 bp, synthetic construct
  misc_feature 1..21: note = SNORD115 artificial/synthetic sequence
  source 1..21: mol_type = other DNA
gaattggacatctggtaataa

SEQ ID NO: 253 - DNA, 21 bp, synthetic construct
  misc_feature 1..21: note = SNORD115 artificial/synthetic sequence
  source 1..21: mol_type = other DNA
gtactgaagtacaagattata

SEQ ID NO: 254 - DNA, 21 bp, synthetic construct
  misc_feature 1..21: note = SNORD115 artificial/synthetic sequence
  source 1..21: mol_type = other DNA
actgtctatgtagagattaaa

SEQ ID NO: 255 - DNA, 21 bp, synthetic construct
  misc_feature 1..21: note = SNORD115 artificial/synthetic sequence
  source 1..21: mol_type = other DNA
ctgtctatgtagagattaaat

SEQ ID NO: 256 - DNA, 21 bp, synthetic construct
  misc_feature 1..21: note = SNORD115 artificial/synthetic sequence
  source 1..21: mol_type = other DNA
tggcattgcagattgaaattt

SEQ ID NO: 257 - DNA, 21 bp, synthetic construct
  misc_feature 1..21: note = SNORD115 artificial/synthetic sequence
  source 1..21: mol_type = other DNA
tcaatttctaagcccattata

SEQ ID NO: 258 - DNA, 21 bp, synthetic construct
  misc_feature 1..21: note = SNORD115 artificial/synthetic sequence
  source 1..21: mol_type = other DNA
caatttctaagcccattatat

SEQ ID NO: 259 - DNA, 21 bp, synthetic construct
  misc_feature 1..21: note = SNORD115 artificial/synthetic sequence
  source 1..21: mol_type = other DNA
tggtatgacagtttcaaataa

SEQ ID NO: 260 - DNA, 21 bp, synthetic construct
  misc_feature 1..21: note = SNORD115 artificial/synthetic sequence
  source 1..21: mol_type = other DNA
acccactgaactaggaaatat

SEQ ID NO: 261 - DNA, 21 bp, synthetic construct
  misc_feature 1..21: note = SNORD115 artificial/synthetic sequence
  source 1..21: mol_type = other DNA
cccactgaactaggaaatatt

SEQ ID NO: 262 - DNA, 21 bp, synthetic construct
  misc_feature 1..21: note = SNORD115 artificial/synthetic sequence
  source 1..21: mol_type = other DNA
agactaacatctctgtaatat

SEQ ID NO: 263 - DNA, 21 bp, synthetic construct
  misc_feature 1..21: note = SNORD115 artificial/synthetic sequence
  source 1..21: mol_type = other DNA
gactaacatctctgtaatata

SEQ ID NO: 264 - DNA, 21 bp, synthetic construct
  misc_feature 1..21: note = SNORD115 artificial/synthetic sequence
  source 1..21: mol_type = other DNA
tgcggaaaggcaaccataaat

SEQ ID NO: 265 - DNA, 21 bp, synthetic construct
  misc_feature 1..21: note = SNORD115 artificial/synthetic sequence
  source 1..21: mol_type = other DNA
gcggaaaggcaaccataaata

SEQ ID NO: 266 - DNA, 21 bp, synthetic construct
  misc_feature 1..21: note = SNORD115 artificial/synthetic sequence
  source 1..21: mol_type = other DNA
cggaaaggcaaccataaatat

SEQ ID NO: 267 - DNA, 21 bp, synthetic construct
  misc_feature 1..21: note = SNORD115 artificial/synthetic sequence
  source 1..21: mol_type = other DNA
ggaaaggcaaccataaatata

SEQ ID NO: 268 - DNA, 21 bp, synthetic construct
  misc_feature 1..21: note = SNORD115 artificial/synthetic sequence
  source 1..21: mol_type = other DNA
ttgtattgcacacccattaaa

SEQ ID NO: 269 - DNA, 21 bp, synthetic construct
  misc_feature 1..21: note = SNORD115 artificial/synthetic sequence
  source 1..21: mol_type = other DNA
tggtgtgcaggcaggtttatt

SEQ ID NO: 270 - DNA, 21 bp, synthetic construct
  misc_feature 1..21: note = SNORD115 artificial/synthetic sequence
  source 1..21: mol_type = other DNA
tgggagactaactactaatta

SEQ ID NO: 271 - DNA, 21 bp, synthetic construct
  misc_feature 1..21: note = SNORD115 artificial/synthetic sequence
  source 1..21: mol_type = other DNA
gggagactaactactaattaa

SEQ ID NO: 272 - DNA, 21 bp, synthetic construct
  misc_feature 1..21: note = SNORD115 artificial/synthetic sequence
  source 1..21: mol_type = other DNA
ttcaagtgtcaactgtatata

SEQ ID NO: 273 - DNA, 21 bp, synthetic construct
  misc_feature 1..21: note = SNORD115 artificial/synthetic sequence
  source 1..21: mol_type = other DNA
tcaagtgtcaactgtatatat

SEQ ID NO: 274 - DNA, 21 bp, synthetic construct
  misc_feature 1..21: note = SNORD115 artificial/synthetic sequence
  source 1..21: mol_type = other DNA
tatgattagcagctgaattta

SEQ ID NO: 275 - DNA, 21 bp, synthetic construct
  misc_feature 1..21: note = SNORD115 artificial/synthetic sequence
  source 1..21: mol_type = other DNA
atgattagcagctgaatttaa

SEQ ID NO: 276 - DNA, 21 bp, synthetic construct
  misc_feature 1..21: note = SNORD115 artificial/synthetic sequence
  source 1..21: mol_type = other DNA
tagaaatgagctacgtaattt

SEQ ID NO: 277 - DNA, 21 bp, synthetic construct
  misc_feature 1..21: note = SNORD115 artificial/synthetic sequence
  source 1..21: mol_type = other DNA
gcaaagaatgagcagttaata

SEQ ID NO: 278 - DNA, 21 bp, synthetic construct
  misc_feature 1..21: note = SNORD115 artificial/synthetic sequence
  source 1..21: mol_type = other DNA
tttcatgtacttccgtatatt

SEQ ID NO: 279 - DNA, 21 bp, synthetic construct
  misc_feature 1..21: note = SNORD115 artificial/synthetic sequence
  source 1..21: mol_type = other DNA
ttacttctctgagggatttat

SEQ ID NO: 280 - DNA, 21 bp, synthetic construct
  misc_feature 1..21: note = SNORD115 artificial/synthetic sequence
  source 1..21: mol_type = other DNA
tacttctctgagggatttatt

SEQ ID NO: 281 - DNA, 21 bp, synthetic construct
  misc_feature 1..21: note = SNORD115 artificial/synthetic sequence
  source 1..21: mol_type = other DNA
ctgtccaggacatccattaaa

SEQ ID NO: 282 - DNA, 21 bp, synthetic construct
  misc_feature 1..21: note = SNORD115 artificial/synthetic sequence
  source 1..21: mol_type = other DNA
tgtccaggacatccattaaat

SEQ ID NO: 283 - DNA, 21 bp, synthetic construct
  misc_feature 1..21: note = SNORD115 artificial/synthetic sequence
  source 1..21: mol_type = other DNA
gtccaggacatccattaaatt

SEQ ID NO: 284 - DNA, 21 bp, synthetic construct
  misc_feature 1..21: note = SNORD115 artificial/synthetic sequence
  source 1..21: mol_type = other DNA
taagagctgacgcccaaatta

SEQ ID NO: 285 - DNA, 21 bp, synthetic construct
  misc_feature 1..21: note = SNORD115 artificial/synthetic sequence
  source 1..21: mol_type = other DNA
ttgatgatgagaaccttatat

SEQ ID NO: 286 - DNA, 21 bp, synthetic construct
  misc_feature 1..21: note = SNORD115 artificial/synthetic sequence
  source 1..21: mol_type = other DNA
gatgatgagaaccttatatta

SEQ ID NO: 287 - DNA, 21 bp, synthetic construct
  misc_feature 1..21: note = SNORD115 artificial/synthetic sequence
  source 1..21: mol_type = other DNA
catgcttcacaaggcaataaa

SEQ ID NO: 288 - DNA, 21 bp, synthetic construct
  misc_feature 1..21: note = SNORD115 artificial/synthetic sequence
  source 1..21: mol_type = other DNA
tcgggagaccagggcttattt

SEQ ID NO: 289 - DNA, 21 bp, synthetic construct
  misc_feature 1..21: note = SNORD115 artificial/synthetic sequence
  source 1..21: mol_type = other DNA
tgacgggattaagagattaaa

SEQ ID NO: 290 - DNA, 21 bp, synthetic construct
  misc_feature 1..21: note = SNORD115 artificial/synthetic sequence
  source 1..21: mol_type = other DNA
agacaggcataggaaataata

SEQ ID NO: 291 - DNA, 21 bp, synthetic construct
  misc_feature 1..21: note = SNORD115 artificial/synthetic sequence
  source 1..21: mol_type = other DNA
gacaggcataggaaataataa

SEQ ID NO: 292 - DNA, 21 bp, synthetic construct
  misc_feature 1..21: note = SNORD115 artificial/synthetic sequence
  source 1..21: mol_type = other DNA
ttgatttgggaagtgataaat

SEQ ID NO: 293 - DNA, 21 bp, synthetic construct
  misc_feature 1..21: note = SNORD115 artificial/synthetic sequence
  source 1..21: mol_type = other DNA
tcaatgatgagaaccttatat

SEQ ID NO: 294 - DNA, 21 bp, synthetic construct
  misc_feature 1..21: note = SNORD115 artificial/synthetic sequence
  source 1..21: mol_type = other DNA
caatgatgagaaccttatatt

SEQ ID NO: 295 - DNA, 21 bp, synthetic construct
  misc_feature 1..21: note = SNORD115 artificial/synthetic sequence
  source 1..21: mol_type = other DNA
gtcgatgatgagaactttata

SEQ ID NO: 296 - DNA, 21 bp, synthetic construct
  misc_feature 1..21: note = SNORD115 artificial/synthetic sequence
  source 1..21: mol_type = other DNA
tcgatgatgagaactttatat

SEQ ID NO: 297 - DNA, 21 bp, synthetic construct
  misc_feature 1..21: note = SNORD115 artificial/synthetic sequence
  source 1..21: mol_type = other DNA
cgatgatgagaactttatatt

SEQ ID NO: 298 - DNA, 21 bp, synthetic construct
  misc_feature 1..21: note = SNORD115 artificial/synthetic sequence
  source 1..21: mol_type = other DNA
tcgatgatgagaaccttatat

SEQ ID NO: 299 - DNA, 21 bp, synthetic construct
  misc_feature 1..21: note = SNORD115 artificial/synthetic sequence
  source 1..21: mol_type = other DNA
cgatgatgagaaccttatatt

SEQ ID NO: 300 - DNA, 21 bp, synthetic construct
  misc_feature 1..21: note = SNORD115 artificial/synthetic sequence
  source 1..21: mol_type = other DNA
cccagcatggccagtatattt

SEQ ID NO: 301 - DNA, 21 bp, synthetic construct
  misc_feature 1..21: note = SNORD115 artificial/synthetic sequence
  source 1..21: mol_type = other DNA
caagaatcgcacacgaattat

SEQ ID NO: 302 - DNA, 21 bp, synthetic construct
  misc_feature 1..21: note = SNORD115 artificial/synthetic sequence
  source 1..21: mol_type = other DNA
gggtcaatgagaaccttatat

SEQ ID NO: 303 - DNA, 21 bp, synthetic construct
  misc_feature 1..21: note = SNORD115 artificial/synthetic sequence
  source 1..21: mol_type = other DNA
ggtcaatgagaaccttatatt

SEQ ID NO: 304 - DNA, 21 bp, synthetic construct
  misc_feature 1..21: note = SNORD115 artificial/synthetic sequence
  source 1..21: mol_type = other DNA
caatgggaccgaccctattaa

SEQ ID NO: 305 - DNA, 21 bp, synthetic construct
  misc_feature 1..21: note = SNORD115 artificial/synthetic sequence
  source 1..21: mol_type = other DNA
gaatgggaccgaccctattaa

SEQ ID NO: 306 - DNA, 21 bp, synthetic construct
  misc_feature 1..21: note = SNORD115 artificial/synthetic sequence
  source 1..21: mol_type = other DNA
agggcagcttccttgttaatt

SEQ ID NO: 307 - DNA, 21 bp, synthetic construct
  misc_feature 1..21: note = SNORD115 artificial/synthetic sequence
  source 1..21: mol_type = other DNA
agttgagttgcagagatttaa

SEQ ID NO: 308 - DNA, 21 bp, synthetic construct
  misc_feature 1..21: note = SNORD115 artificial/synthetic sequence
  source 1..21: mol_type = other DNA
atttctggtgtgtgcttataa

SEQ ID NO: 309 - DNA, 21 bp, synthetic construct
  misc_feature 1..21: note = SNORD115 artificial/synthetic sequence
  source 1..21: mol_type = other DNA
tttctggtgtgtgcttataaa

SEQ ID NO: 310 - DNA, 21 bp, synthetic construct
  misc_feature 1..21: note = SNORD115 artificial/synthetic sequence
  source 1..21: mol_type = other DNA
tgcctggatgggttcatttaa

SEQ ID NO: 311 - DNA, 21 bp, synthetic construct
  misc_feature 1..21: note = SNORD115 artificial/synthetic sequence
  source 1..21: mol_type = other DNA
gcctggatgggttcatttaat

SEQ ID NO: 312 - DNA, 21 bp, synthetic construct
  misc_feature 1..21: note = SNORD115 artificial/synthetic sequence
  source 1..21: mol_type = other DNA
ggagttgagctgcagatattt

SEQ ID NO: 313 - DNA, 21 bp, synthetic construct
  misc_feature 1..21: note = SNORD115 artificial/synthetic sequence
  source 1..21: mol_type = other DNA
gagttgagctgcagatattta

SEQ ID NO: 314 - DNA, 21 bp, synthetic construct
  misc_feature 1..21: note = SNORD115 artificial/synthetic sequence
  source 1..21: mol_type = other DNA
agttgagctgcagatatttaa

SEQ ID NO: 315 - DNA, 21 bp, synthetic construct
  misc_feature 1..21: note = SNORD115 artificial/synthetic sequence
  source 1..21: mol_type = other DNA
taaggatcacacaggatttat

SEQ ID NO: 316 - DNA, 21 bp, synthetic construct
  misc_feature 1..21: note = SNORD115 artificial/synthetic sequence
  source 1..21: mol_type = other DNA
tcgatgatgagaaacttatat

SEQ ID NO: 317 - DNA, 21 bp, synthetic construct
  misc_feature 1..21: note = SNORD115 artificial/synthetic sequence
  source 1..21: mol_type = other DNA
cgatgatgagaaacttatatt

SEQ ID NO: 318 - DNA, 21 bp, synthetic construct
  misc_feature 1..21: note = SNORD115 artificial/synthetic sequence
  source 1..21: mol_type = other DNA
gaagaggatgctgggaattat

SEQ ID NO: 319 - DNA, 21 bp, synthetic construct
  misc_feature 1..21: note = SNORD115 artificial/synthetic sequence
  source 1..21: mol_type = other DNA
gaatgggacaaaccctaataa

SEQ ID NO: 320 - DNA, 21 bp, synthetic construct
  misc_feature 1..21: note = SNORD115 artificial/synthetic sequence
  source 1..21: mol_type = other DNA
atttctggtgtgagcttataa

SEQ ID NO: 321 - DNA, 21 bp, synthetic construct
  misc_feature 1..21: note = SNORD115 artificial/synthetic sequence
  source 1..21: mol_type = other DNA
tttctggtgtgagcttataaa

SEQ ID NO: 322 - DNA, 21 bp, synthetic construct
  misc_feature 1..21: note = SNORD115 artificial/synthetic sequence
  source 1..21: mol_type = other DNA
tggtcctgtcccaggaattta

SEQ ID NO: 323 - DNA, 21 bp, synthetic construct
  misc_feature 1..21: note = SNORD115 artificial/synthetic sequence
  source 1..21: mol_type = other DNA
agtcgagctgtggagatttaa

SEQ ID NO: 324 - DNA, 21 bp, synthetic construct
  misc_feature 1..21: note = SNORD115 artificial/synthetic sequence
  source 1..21: mol_type = other DNA
cctggcagggccactataatt

SEQ ID NO: 325 - DNA, 21 bp, synthetic construct
  misc_feature 1..21: note = SNORD115 artificial/synthetic sequence
  source 1..21: mol_type = other DNA
ctggcagggccactataattt

SEQ ID NO: 326 - DNA, 21 bp, synthetic construct
  misc_feature 1..21: note = SNORD115 artificial/synthetic sequence
  source 1..21: mol_type = other DNA
gatgatgagaaccttatattt

SEQ ID NO: 327 - DNA, 21 bp, synthetic construct
  misc_feature 1..21: note = SNORD115 artificial/synthetic sequence
  source 1..21: mol_type = other DNA
caatgatgagaaccctatatt

SEQ ID NO: 328 - DNA, 21 bp, synthetic construct
  misc_feature 1..21: note = SNORD115 artificial/synthetic sequence
  source 1..21: mol_type = other DNA
cccaacctaggtgagaaattt

SEQ ID NO: 329 - DNA, 21 bp, synthetic construct
  misc_feature 1..21: note = SNORD115 artificial/synthetic sequence
  source 1..21: mol_type = other DNA
tcagtgtcgagaaccttatat

SEQ ID NO: 330 - DNA, 21 bp, synthetic construct
  misc_feature 1..21: note = SNORD115 artificial/synthetic sequence
  source 1..21: mol_type = other DNA
cagtgtcgagaaccttatatt

SEQ ID NO: 331 - DNA, 21 bp, synthetic construct
  misc_feature 1..21: note = SNORD115 artificial/synthetic sequence
  source 1..21: mol_type = other DNA
cctggcagggcccttatattt

SEQ ID NO: 332 - DNA, 21 bp, synthetic construct
  misc_feature 1..21: note = SNORD115 artificial/synthetic sequence
  source 1..21: mol_type = other DNA
tcgattatgagaaccttatat

SEQ ID NO: 333 - DNA, 21 bp, synthetic construct
  misc_feature 1..21: note = SNORD115 artificial/synthetic sequence
  source 1..21: mol_type = other DNA
cgattatgagaaccttatatt

SEQ ID NO: 334 - DNA, 21 bp, synthetic construct
  misc_feature 1..21: note = SNORD115 artificial/synthetic sequence
  source 1..21: mol_type = other DNA
tcagtgatgagaaccttatat

SEQ ID NO: 335 - DNA, 21 bp, synthetic construct
  misc_feature 1..21: note = SNORD115 artificial/synthetic sequence
  source 1..21: mol_type = other DNA
cagtgatgagaaccttatatt

SEQ ID NO: 336 - DNA, 21 bp, synthetic construct
  misc_feature 1..21: note = SNORD115 artificial/synthetic sequence
  source 1..21: mol_type = other DNA
ctgatgatgagaaccttatat

SEQ ID NO: 337 - DNA, 21 bp, synthetic construct
  misc_feature 1..21: note = SNORD115 artificial/synthetic sequence
  source 1..21: mol_type = other DNA
ccagctagaccacacttattt

SEQ ID NO: 338 - DNA, 21 bp, synthetic construct
  misc_feature 1..21: note = SNORD115 artificial/synthetic sequence
  source 1..21: mol_type = other DNA
aggacgttgggatcctattat

SEQ ID NO: 339 - DNA, 21 bp, synthetic construct
  misc_feature 1..21: note = SNORD115 artificial/synthetic sequence
  source 1..21: mol_type = other DNA
ttctgaagagaggtgattatt

SEQ ID NO: 340 - DNA, 21 bp, synthetic construct
  misc_feature 1..21: note = SNORD115 artificial/synthetic sequence
  source 1..21: mol_type = other DNA
tctgaagagaggtgattattt

SEQ ID NO: 341 - DNA, 21 bp, synthetic construct
  misc_feature 1..21: note = SNORD115 artificial/synthetic sequence
  source 1..21: mol_type = other DNA
ctgaagagaggtgattattta

SEQ ID NO: 342 - DNA, 21 bp, synthetic construct
  misc_feature 1..21: note = SNORD115 artificial/synthetic sequence
  source 1..21: mol_type = other DNA
tgaagagaggtgattatttaa

SEQ ID NO: 343 - DNA, 21 bp, synthetic construct
  misc_feature 1..21: note = SNORD115 artificial/synthetic sequence
  source 1..21: mol_type = other DNA
gaagagaggtgattatttaaa

SEQ ID NO: 344 - DNA, 21 bp, synthetic construct
  misc_feature 1..21: note = SNORD115 artificial/synthetic sequence
  source 1..21: mol_type = other DNA
ggttggattgtggagattaaa

SEQ ID NO: 345 - DNA, 21 bp, synthetic construct
  misc_feature 1..21: note = SNORD115 artificial/synthetic sequence
  source 1..21: mol_type = other DNA
ggcccagcctaggtgataatt

SEQ ID NO: 346 - DNA, 21 bp, synthetic construct
  misc_feature 1..21: note = SNORD115 artificial/synthetic sequence
  source 1..21: mol_type = other DNA
gcccagcctaggtgataattt

SEQ ID NO: 347 - DNA, 21 bp, synthetic construct
  misc_feature 1..21: note = SNORD115 artificial/synthetic sequence
  source 1..21: mol_type = other DNA
cgttgggtctcagagatttaa

SEQ ID NO: 348 - DNA, 21 bp, synthetic construct
  misc_feature 1..21: note = SNORD115 artificial/synthetic sequence
  source 1..21: mol_type = other DNA
cccaccctaagtgagaaattt

SEQ ID NO: 349 - DNA, 21 bp, synthetic construct
  misc_feature 1..21: note = SNORD115 artificial/synthetic sequence
  source 1..21: mol_type = other DNA
tcaatgatgagaaccttataa

SEQ ID NO: 350 - DNA, 21 bp, synthetic construct
  misc_feature 1..21: note = SNORD115 artificial/synthetic sequence
  source 1..21: mol_type = other DNA
caatgatgagaaccttataat

SEQ ID NO: 351 - DNA, 21 bp, synthetic construct
  misc_feature 1..21: note = SNORD115 artificial/synthetic sequence
  source 1..21: mol_type = other DNA
atgatgagaaccttgtattat

SEQ ID NO: 352 - DNA, 21 bp, synthetic construct
  misc_feature 1..21: note = SNORD115 artificial/synthetic sequence
  source 1..21: mol_type = other DNA
cccaacagggccaccatattt

SEQ ID NO: 353 - DNA, 21 bp, synthetic construct
  misc_feature 1..21: note = SNORD115 artificial/synthetic sequence
  source 1..21: mol_type = other DNA
cattggtgacaagtcaatatt

SEQ ID NO: 354 - DNA, 21 bp, synthetic construct
  misc_feature 1..21: note = SNORD115 artificial/synthetic sequence
  source 1..21: mol_type = other DNA
attggtgacaagtcaatattt

SEQ ID NO: 355 - DNA, 21 bp, synthetic construct
  misc_feature 1..21: note = SNORD115 artificial/synthetic sequence
  source 1..21: mol_type = other DNA
tgcagtcccagaatgtaattt

SEQ ID NO: 356 - DNA, 21 bp, synthetic construct
  misc_feature 1..21: note = SNORD115 artificial/synthetic sequence
  source 1..21: mol_type = other DNA
ccaggcagggccactatattt

SEQ ID NO: 357 - DNA, 21 bp, synthetic construct
  misc_feature 1..21: note = SNORD115 artificial/synthetic sequence
  source 1..21: mol_type = other DNA
cctggcagggccactatattt

SEQ ID NO: 358 - DNA, 21 bp, synthetic construct
  misc_feature 1..21: note = SNORD115 artificial/synthetic sequence
  source 1..21: mol_type = other DNA
ttgcggtcatgggccataaat

SEQ ID NO: 359 - DNA, 21 bp, synthetic construct
  misc_feature 1..21: note = SNORD115 artificial/synthetic sequence
  source 1..21: mol_type = other DNA
agcagctcccagctgaaattt

SEQ ID NO: 360 - DNA, 21 bp, synthetic construct
  misc_feature 1..21: note = SNORD115 artificial/synthetic sequence
  source 1..21: mol_type = other DNA
agggacctgtcctggaaattt

SEQ ID NO: 361 - DNA, 50 bp, synthetic construct
  misc_feature 1..50: note = shRNA artificial/synthetic sequence
  variation 22..29: note = some n may or may not be absent: "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..50: mol_type = other DNA
tgatgatgagaaccttatattnnnnnnnnaatataaggttctcatcatca

SEQ ID NO: 362-365 - skipped (4 entries)

SEQ ID NO: 366 - DNA, 46 bp, synthetic construct
  misc_feature 1..46: note = shRNA artificial/synthetic sequence
  source 1..46: mol_type = other DNA
gatgatgagaaccttatattctcgagaatataaggttctcatcatc

SEQ ID NO: 367 - DNA, 44 bp, synthetic construct
  misc_feature 1..44: note = shRNA artificial/synthetic sequence
  source 1..44: mol_type = other DNA
atgatgagaaccttatattctcgagaatataaggttctcatcat

SEQ ID NO: 368 - DNA, 42 bp, synthetic construct
  misc_feature 1..42: note = shRNA artificial/synthetic sequence
  source 1..42: mol_type = other DNA
tgatgagaaccttatattctcgagaatataaggttctcatca

SEQ ID NO: 369 - DNA, 40 bp, synthetic construct
  misc_feature 1..40: note = shRNA artificial/synthetic sequence
  source 1..40: mol_type = other DNA
gatgagaaccttatattctcgagaatataaggttctcatc

SEQ ID NO: 370 - DNA, 46 bp, synthetic construct
  misc_feature 1..46: note = shRNA artificial/synthetic sequence
  source 1..46: mol_type = other DNA
tgatgatgagaaccttatatctcgagatataaggttctcatcatca

SEQ ID NO: 371 - DNA, 44 bp, synthetic construct
  misc_feature 1..44: note = shRNA artificial/synthetic sequence
  source 1..44: mol_type = other DNA
tgatgatgagaaccttatactcgagtataaggttctcatcatca

SEQ ID NO: 372 - DNA, 42 bp, synthetic construct
  misc_feature 1..42: note = shRNA artificial/synthetic sequence
  source 1..42: mol_type = other DNA
tgatgatgagaaccttatctcgagataaggttctcatcatca

SEQ ID NO: 373 - DNA, 40 bp, synthetic construct
  misc_feature 1..40: note = shRNA artificial/synthetic sequence
  source 1..40: mol_type = other DNA
tgatgatgagaaccttactcgagtaaggttctcatcatca

SEQ ID NO: 374 - DNA, 48 bp, synthetic construct
  misc_feature 1..48: note = shRNA artificial/synthetic sequence
  variation 21..28: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..48: mol_type = other DNA
gatgatgagaaccttatattnnnnnnnnaatataaggttctcatcatc

SEQ ID NO: 375 - DNA, 46 bp, synthetic construct
  misc_feature 1..46: note = shRNA artificial/synthetic sequence
  variation 20..27: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..46: mol_type = other DNA
atgatgagaaccttatattnnnnnnnnaatataaggttctcatcat

SEQ ID NO: 376 - DNA, 44 bp, synthetic construct
  misc_feature 1..44: note = shRNA artificial/synthetic sequence
  variation 19..26: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..44: mol_type = other DNA
tgatgagaaccttatattnnnnnnnnaatataaggttctcatca

SEQ ID NO: 377 - DNA, 42 bp, synthetic construct
  misc_feature 1..42: note = shRNA artificial/synthetic sequence
  variation 18..25: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..42: mol_type = other DNA
gatgagaaccttatattnnnnnnnnaatataaggttctcatc

SEQ ID NO: 378 - DNA, 48 bp, synthetic construct
  misc_feature 1..48: note = shRNA artificial/synthetic sequence
  variation 21..28: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..48: mol_type = other DNA
tgatgatgagaaccttatatnnnnnnnnatataaggttctcatcatca

SEQ ID NO: 379 - DNA, 46 bp, synthetic construct
  misc_feature 1..46: note = shRNA artificial/synthetic sequence
  variation 20..27: note = some n may or may not be present; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..46: mol_type = other DNA
tgatgatgagaaccttatannnnnnnntataaggttctcatcatca

SEQ ID NO: 380 - DNA, 44 bp, synthetic construct
  misc_feature 1..44: note = shRNA artificial/synthetic sequence
  variation 19..26: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..44: mol_type = other DNA
tgatgatgagaaccttatnnnnnnnnataaggttctcatcatca

SEQ ID NO: 381 - DNA, 42 bp, synthetic construct
  misc_feature 1..42: note = shRNA artificial/synthetic sequence
  variation 18..25: note = some n may or may not be absent; "nnnnnnnn" can be CTCGAG (SEQ ID NO: 362), TCAAGAG (SEQ ID NO: 363), TTCG (SEQ ID NO: 364) or GAAGCTTG (SEQ ID NO: 365)
  source 1..42: mol_type = other DNA
tgatgatgagaaccttannnnnnnntaaggttctcatcatca

SEQ ID NO: 382 - DNA, 48 bp, synthetic construct
  misc_feature 1..48: note = shRNA artificial/synthetic sequence
  source 1..48: mol_type = other DNA
caatgatgagaaccgtatattctcgagaatatacggttctcatcattg

SEQ ID NO: 383 - DNA, 48 bp, synthetic construct
  misc_feature 1..48: note = shRNA artificial/synthetic sequence
  source 1..48: mol_type = other DNA
ggtaacctgttctccaaatttctcgagaaatttggagaacaggttacc

(19) United States
(12) Patent Application Publication
(10) Pub. No.: US 2023/0332150 A1
(43) Pub. Date: Oct. 19, 2023

(54) shRNA TARGETING SNORD115 LOCATIONS TO RESTORE PATERNAL UBE3A GENE EXPRESSION IN ANGELMAN SYNDROME

(71) Applicants: University of Connecticut, Farmington, CT (US); Ovid Therapeutics Inc., New York, NY (US)

(72) Inventors: Stormy CHAMBERLAIN, Oxford, CT (US); Noelle Germain, Watertown, CT (US); Peter Perrino, Glastonbury, CT (US)

(21) Appl. No.: 18/179,716

(22) Filed: Mar. 7, 2023

Related U.S. Application Data
(60) Provisional application No. 63/317,155, filed on Mar. 7, 2022.

(51) Int. Cl. C12N 15/113; C12N 15/86

(52) U.S. Cl. CPC C12N 15/113; C12N 15/86; C12N 15/1137; C12N 2310/122; C12N 2310/531; C12N 2740/15043

(57) ABSTRACT

Provided herein are compositions and methods for activating expression from the paternally-inherited allele of UBE3A in Angelman syndrome using viral vector delivery of short hairpin RNAs. Provided herein are compositions and methods for reducing or eliminating expression of SNORD115 and UBE3A-ATS in Angelman syndrome using viral vector delivery of short hairpin RNAs.

### CROSS-REFERENCE TO RELATED APPLICATION

This application claims benefit and priority to U.S. Provisional Application No. 63/317,155, filed Mar. 7, 2022, which is incorporated herein by reference in its entirety.

### STATEMENT OF GOVERNMENT SUPPORT

This invention was made with government support under Contract No. 1R01HD094953 awarded by the National Institutes of Health. The government has certain rights in the invention.

### TECHNICAL FIELD

The present disclosure relates to compositions and methods for activating expression from the paternally-inherited allele of UBE3A in subjects having Angelman syndrome using short hairpin RNAs.

### REFERENCE TO SEQUENCE LISTING

The application contains a Sequence Listing which has been submitted electronically in .XML format and is hereby incorporated by reference in its entirety. Said .XML copy, created on Apr. 7, 2023, is named “2262-99.xml” and is 674,972 bytes in size. The sequence listing contained in this .XML file is part of the specification and is hereby incorporated by reference herein in its entirety.

### BACKGROUND

Angelman syndrome (AS) is a neurodevelopmental disorder affecting ˜1/15,000 individuals. Individuals with AS have developmental delay, severe cognitive impairment, ataxic gait, frequent seizures, short attention span, absent speech, and characteristic happy demeanor. Neurons derived from induced pluripotent stem cells (iPSC) from AS patients exhibit a depolarized resting membrane potential, delayed action potential development, and reduced spontaneous synaptic activity. Fink et al., 2017, Nat Commun 8. AS affects a relatively large patient population; a contact registry with >3,000 patients has been established and ˜250 new diagnoses of AS are made each year. Individuals with AS require life-long care.

AS is caused by loss of function from the maternal copy of UBE3A, a gene encoding an E3 ubiquitin ligase. This loss of function mutation can be caused by any type of gene mutation in the maternal allele. UBE3A is expressed exclusively from the maternal allele in neurons. All individuals with AS have a normal paternal UBE3A allele that is epigenetically silenced in neurons in cis by a long, non-coding RNA, called UBE3A antisense transcript (UBE3A-ATS) (Rougeulle et al., 1997, Nat Genet 17, 14-15; Chamberlain and Brannan, 2001, Genomics 73, 316-322). Reactivation of the paternal allele has been shown to restore UBE3A protein expression and alleviate behavioral deficits in an AS mouse model. The restoration of UBE3A expression in humans is expected to ameliorate the disease, especially if it is restored in infants.

### SUMMARY

Provided herein is a novel treatment for Angelman syndrome by inhibiting the silencing of paternal UBE3A and allowing expression of paternal UBE3A from its native regulatory elements, thus replacing or augmenting missing maternal UBE3A. Increased expression of UBE3A in neurons is accomplished by interfering with transcription of SNORD115 and/or UBE3A-ATS. Since the native regulatory elements control expression, overexpression of UBE3A is prevented. This approach can improve AS symptoms through a single treatment and eliminate the need for multiple treatments.

Provided herein is a polynucleotide sequence including:
5′-TGATGATGAGAACCTTATATTCTCGAGAATATAAGGTTCTCATCATCA -3′ (SEQ ID No: 3). Expression vectors including SEQ ID NO: 3 are provided. In embodiments, the expression vector is an adeno-associated viral (AAV) vector or a lentiviral vector. Pharmaceutical compositions including the foregoing are provided.

Provided herein is a polynucleotide encoding a shRNA including a nucleotide sequence that is at least 85%, at least 90%, at least 95%, or 100% complementary to a RNA encoded by any of SEQ ID NOs: 19-360. In embodiments, the polynucleotide is SEQ ID NO: 3. In embodiments, the shRNA causes activation of, or an increase in, expression of paternal UBE3A. In embodiments, the shRNA causes a reduction of expression of paternal SNORD115 and UBE3A-ATS. Expression vectors including the shRNA are provided. In embodiments, the expression vector is an adeno-associated viral (AAV) vector or a lentiviral vector. Pharmaceutical compositions including the foregoing are provided.

Provided herein is a method of treating Angelman syndrome including administering to a patient in need thereof the polynucleotide of SEQ ID NO: 3. In embodiments, the polynucleotide of SEQ ID NO: 3 encodes a shRNA which causes a reduction of expression of paternal SNORD115 and UBE3A-ATS. In embodiments, the polynucleotide of SEQ ID NO: 3 encodes a shRNA which causes activation of, or an increase in, expression of paternal UBE3A gene.

Provided herein is a method of treating Angelman syndrome including administering to a patient in need thereof a polynucleotide encoding a shRNA including a nucleotide sequence that is at least 85%, at least 90%, at least 95%, or 100% complementary to a RNA encoded by any of SEQ ID NOs: 19-360. In embodiments, the polynucleotide is SEQ ID NO: 3. In embodiments, the shRNA causes activation of, or an increase in, expression of paternal UBE3A. In embodiments, the shRNA causes a reduction of expression of paternal SNORD115 and UBE3A-ATS.

In embodiments, SEQ ID NO: 3 encodes a shRNA capable of inhibiting the silencing of paternal UBE3A. In embodiments, the SEQ ID NO: 3 is contained within an expression vector. In embodiments, the expression vector is an adeno-associated viral (AAV) vector or a lentiviral vector. In embodiments, a method is provided of inhibiting the silencing of paternal UBE3A gene by the RNA antisense transcript encoded by UBE3A-ATS (SEQ ID NO: 1) and SNORD115 (SEQ ID NO: 2) which includes administering to a patient in need thereof an amount of SEQ ID NO: 3 which is effective to cut the RNA antisense transcript encoded by SEQ ID NO: 2.

In embodiments, a method is provided of inhibiting the silencing of paternal UBE3A gene by the RNA antisense transcript encoded by SEQ ID NO: 1 which includes administering to a patient in need thereof, an amount of a shRNA including a nucleotide sequence that is at least 85%, at least 90%, at least 95%, or 100% complementary to a RNA encoded by any of SEQ ID NOs: 19-360, which is effective to cut the RNA antisense transcript encoded by SEQ ID NO: 2.

In embodiments, a shRNA provided herein is encoded by a portion of SEQ ID NO: 3, e.g., having the bold nucleotides, which has been shortened by one, two, three or four nucleotides at either end of the bold nucleotides. Likewise, in embodiments, the shRNA provided herein can contain a portion of SEQ ID NO: 3, e.g., having the italicized nucleotides, which has been shortened by one, two or three nucleotides at either end of the italicized nucleotides. In embodiments, a shRNA provided herein is encoded by a polynucleotide including any of SEQ ID NOs: 19-360 which has been shortened by one, two, three or four nucleotides at either end.

In embodiments, a polynucleotide sequence is provided as follows:

(SEQ ID NO: 361)
5′-TGATGATGAGAACCTTATATTnnnnnnnnAATATAAGGTTCTCATC
ATCA-3′, wherein nnnnnnnn can be
(SEQ ID NO: 362)
CTCGAG,
(SEQ ID NO: 363)
TCAAGAG,
(SEQ ID NO: 364)
TTCG
or
(SEQ ID NO: 365)
GAAGCTTG.

In embodiments, a polynucleotide sequence is provided which includes a first portion, a second portion and a third portion, the first portion comprising any of SEQ ID NOs: 19-360, the second portion comprising any of SEQ ID Nos: 362, 363, 364, or 365, and the third portion comprising respective nucleotide sequences complementary to those in SEQ ID NOs: 19-360.

### BRIEF DESCRIPTION OF THE DRAWINGS

FIG. 1 shows chromosomal mutations in Angelman Syndrome.

FIG. 2 shows a diagram of paternal SNHG14 and UBE3A gene.

FIG. 3A and FIG. 3B show genomic locations of shRNA targets (solid callout).

UCSC Genome Browser view of the 15q11-q13 region containing the imprinted SNHG14/UBE3A locus (dashed-line callout). Location of shRNA targets within the SNORD115 snoRNA cluster.

FIG. 4 is a bar graph showing qRT-PCR analysis of Angelman syndrome hESC-derived neurons following treatment with SNHG14-targeting shRNAs (SNORD115 shRNA 1, SNORD115 shRNA 2 and SNORD115 shRNA 3) or non-targeting control shRNA (SCRAM). SNORD115 shRNA 3 knocked down SNORD115 and UBE3A-ATS and activated paternal UBE3A.

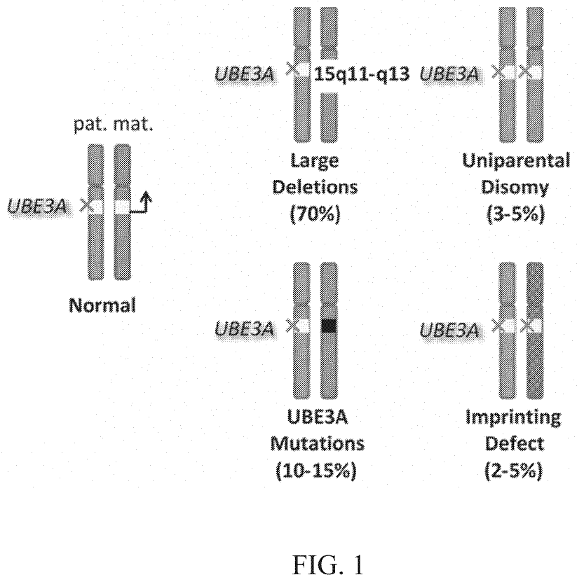

### DETAILED DESCRIPTION

UBE3A is a gene which encodes the E3 ubiquitin ligase. The genomic coordinates for UBE3A are hg19 chr15:25,582,381-25,684,175 on the minus strand. There are three normal isoforms of UBE3A: Isoform 1 (accession number X98032); Isoform 2 (accession number X98031); and isoform 3 (Accession number X98033). In neurons, UBE3A is expressed exclusively from the maternal allele. The paternal UBE3A allele is epigenetically silenced by the long, non-coding RNA UBE3A antisense transcript (UBE3A-ATS) encoded by SEQ ID NO: 1. The genomic coordinates for UBE3A-ATS are hg19 chr15:25,223,730-25,664,609 on the plus strand. The following genomic coordinates are of interest: hg19 chr15:25,522,751-25,591,391 on the plus strand.

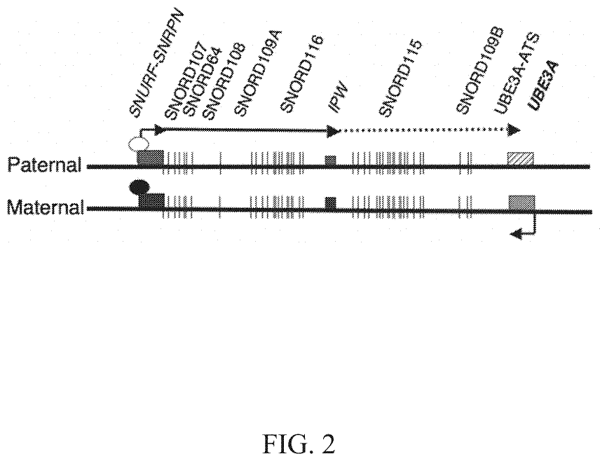

UBE3A-ATS/Ube3a-ATS (human/mouse) is the antisense RNA that is transcribed as part of a larger transcript called SNHG14 (SNORNA HOST GENE 14) near the UBE3A locus. Human UBE3A-ATS is expressed as a part of SNHG14 exclusively from the paternal allele in the central nervous system (CNS). The transcript is about 600 kb long, starts at SNURF-SNRPN and extends into the first intron of UBE3A on the opposite strand. See, e.g., FIG. 2. The promoter for SNURF/SNRPN is the Prader-Willi syndrome Imprinting Center (PWS-IC). SNHG14 (Small Nucleolar RNA Host Gene 14) is an RNA gene and is affiliated with the lncRNA class. UBE3A-ATS is part of SNHG14.

SNHG14 is located within the Prader-Willi critical region and produces a long, spliced maternally-imprinted RNA that initiates at one of several promoters shared by the SNRPN (small nuclear ribonucleoprotein polypeptide N) and SNURF genes. This transcript serves as a host RNA for the small nucleolar RNA, C/D box 115 and 116 clusters. See, Runte et al., 2001, Hum Mol Genet 10, 2687-2700. This RNA extends in antisense into the region of the UBE3A gene and is thought to regulate imprinted expression of UBE3A in the brain. The main promoter of SNURF-SNRPN is the PWS-IC and about 35 kb upstream of the PWS-IC is the AS-IC. These two regions are thought to control the expression of the entire SNHG14 transcript. Starting at the promoter, the entire transcript can be transcribed and after transcription is further processed and spliced.

SNURF/SNRPN is a bicistronic gene that encodes two protein-coding transcripts, SNURF and SNRPN. Both SNURF and SNRPN proteins localize to the cell nucleus. SNRPN is a small nuclear ribonucleoprotein, and the function of SNURF is unknown. The transcript that begins at SNRPN/SNURF also continues past these genes, and within its introns are sequences for several C/D box snoRNAs. Box C/D small nucleolar RNAs (SNORDs) represent a well-defined family of small non-coding RNAs that exert their regulatory functions via antisense-based mechanisms. Most C/D box snoRNAs function in non-mRNA methylation.

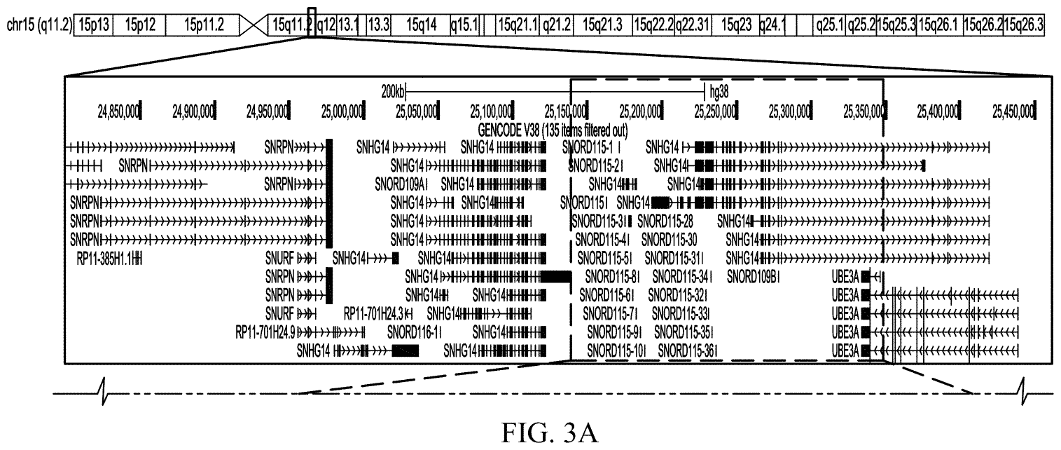

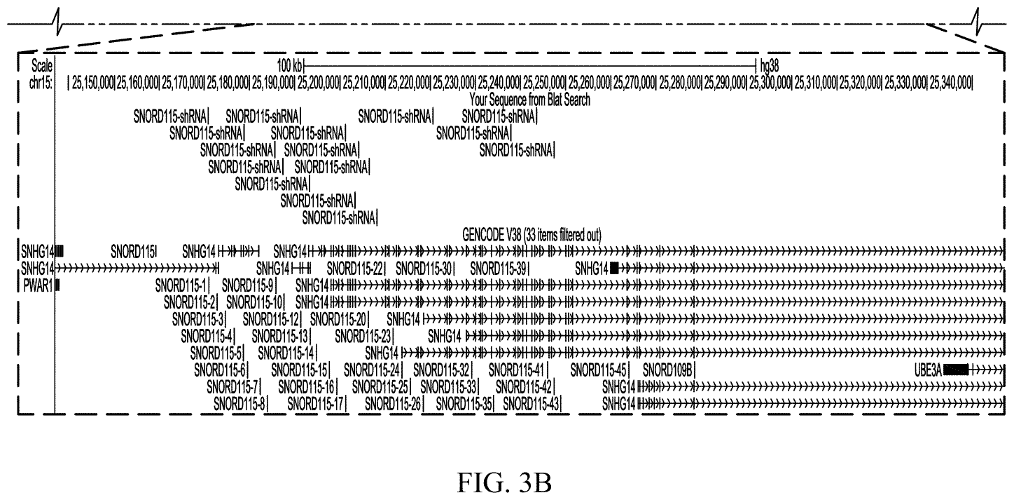

Many orphan SNORDs are generated from two large, imprinted chromosomal domains at human 15q11q13 and 14q32. See, e.g., FIG. 3. As indicated above, the imprinted human 15q11q13 region—also known as the Prader-Willi Syndrome (PWS)/Angelman Syndrome (AS) locus or SNURF-SNRPN domain—contains several paternally expressed, protein coding genes as well as numerous paternally expressed, neuronal-specific SNORD genes organized as two main repetitive DNA arrays: the SNORD116 and SNORD115 clusters composed of 29 and 47 related gene copies, respectively.

SNORD115 encodes a small nucleolar RNA (snoRNA) that is found clustered with dozens of other similar snoRNAs on chromosome 15. These genes are found mostly within introns of the SNURF-SNRPN/SNHG14 transcript, which is maternally imprinted and expressed from the PWS/AS region. The genomic coordinates for SNORD115 are >hg38_dna range=chr15:25159221-25269858.

The compositions and methods described herein are drawn to targeting SNORD115 and UBE3A-ATS to unsilence the paternal UBE3A allele. Effective inhibition of SNORD115 and UBE3A-ATS by short hairpin RNAs (shRNA) described herein result in a reduction in SNORD115 and UBE3A-ATS expression levels and a concomitant increase in the expression levels of the paternal UBE3A allele. The shRNAs described herein were targeted to cut at single locations or multiple locations within the RNA expressed from the SNORD115 locus and were tested in H9-AS (hESC)-derived neurons engineered to imprint early during neurogenesis. SNORD115 shRNA 3 (SEQ ID NO: 3) is a shRNA that uniquely cleaves an RNA transcript in multiple places, i.e., targeting multiple sequences within the SNORD115 cluster (see, FIG. 3), thus increasing the likelihood that the shRNA cleaves the transcript and activates paternal UBE3A, thereby providing a therapeutic approach to treating Angelman syndrome. Out of the total cluster of 48 individual annotated snoRNAs, SNORD115 shRNA 3 has homology to 15 of them: SNORD115-1 (SEQ ID NO: 4), SNORD115-5 (SEQ ID NO: 5), SNORD115-9 (SEQ ID NO: 6), SNORD115-10 (SEQ ID NO: 7), SNORD115-12 (SEQ ID NO: 8), SNORD115-13 (SEQ ID NO: 9), SNORD115-17 (SEQ ID NO: 10), SNORD115-18 (SEQ ID NO: 11), SNORD115-19 (SEQ ID NO: 12), SNORD115-20 (SEQ ID NO: 13), SNORD115-21 (SEQ ID NO: 14), SNORD115-27 (SEQ ID NO: 15), SNORD115-37 (SEQ ID NO: 16), SNORD115-40 (SEQ ID NO: 17), SNORD115-42 (SEQ ID NO: 18). The underlined portions of the sequences highlight target areas.

In embodiments, compositions and methods herein relate to the treatment or prevention of AS. A patient in need of such treatment or prevention has AS or is at risk for developing AS. As used herein, the term “patient in need” includes any mammal in need of these methods of treatment or prophylaxis, including humans. The subject may be male or female. In certain aspects, the patient in need, having AS, treated according to the methods and compositions provided herein may show an improvement in anxiety, learning, balance, motor function, and/or seizures, or the method may return the neuronal resting membrane potential to about −70 mV, ameliorate the action potential development delay, increase spontaneous synaptic activity, and may ameliorate additional alterations in the neuronal phenotype relating to rheobase, action potential characteristics (e.g. shape), membrane current, synaptic potentials, and/or ion channel conductance.

In embodiments, a polynucleotide includes a first nucleotide sequence encoding a short hairpin RNA (shRNA) that interferes with expression of the SNORD115 sequence (SEQ ID NO: 2). In embodiments, a polynucleotide includes a first nucleotide sequence encoding a short hairpin RNA (shRNA) that results in decreased expression of the UBE3A-ATS sequence (SEQ ID NO: 1). For example, a portion of the shRNAs described herein may be complementary to the RNA sequence encoded by SEQ ID NO: 2 or a sequence contained therein. For example, a portion of the shRNAs described herein may be complementary to the RNA sequence encoded by SEQ ID NO: 3 or a sequence contained therein. In embodiments, the shRNAs described herein are RNA polynucleotides encoded by a first nucleotide sequence. The polynucleotide encompassing the first nucleotide sequence may be a DNA polynucleotide suitable for cloning into an appropriate vector (e.g., a plasmid) for culturing and subsequent production of viral particles. In turn, viral particles may contain the DNA polynucleotide with the nucleotide coding sequence in a form suitable for infection. Thus, the first nucleotide sequence may be a DNA sequence cloned into a plasmid for viral particle production or encapsulated in a viral particle. As retroviruses carry nucleotide coding sequences in the form of RNA polynucleotides, retroviral particles (e.g., lentivirus) contain an RNA polynucleotide that includes the first nucleotide sequence as a corresponding RNA sequence.

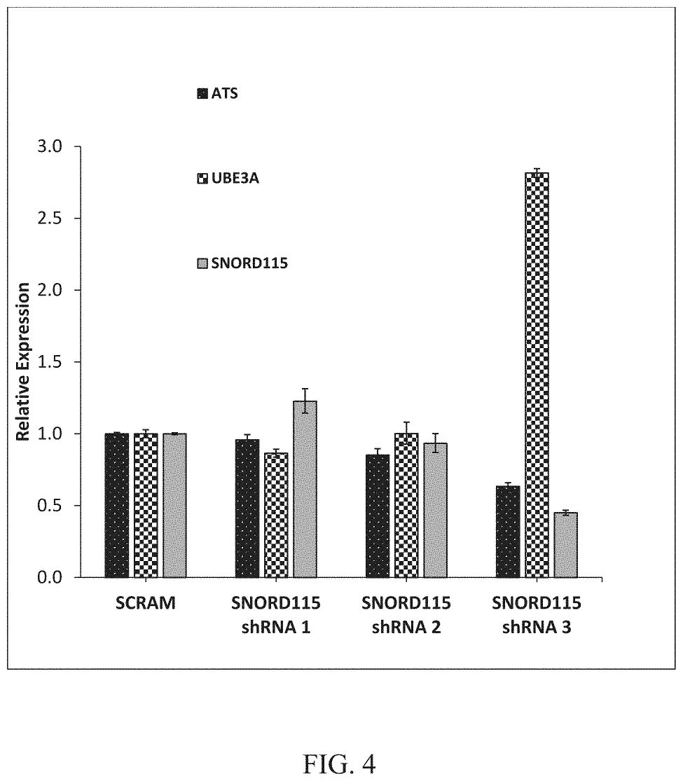

Disclosed herein are novel shRNAs that cut SNORD115 thereby reducing UBE3A-ATS expression and, in turn activate, the paternally inherited copy of UBE3A in neurons. This provides the UBE3A gene product in a cell type that is missing the protein in Angelman syndrome. There is a potential search space of about −60 kb in the genomic LNCAT sequence which may provide potential shRNA targets. However, not every predicted sequence actually reduces SNORD115 and/or UBE3A-ATS and restores UBE3A. Accordingly, as shown by the certain examples herein, it is difficult to predict which sequences will or will not work. See, e.g., FIG. 4.

The first nucleotide sequence encodes a shRNA. For example, the first nucleotide sequence may be SEQ ID NO: 3
(5′-TGATGATGAGAACCTTATATTCTCGAGAATATAAGGTTCTCATCATCA -3′). The first nucleotide sequence may also be a modified SEQ ID NO: 3 having the bold nucleotides in SEQ ID NO: 3 replaced by any of SEQ ID NOs: 19-360 and the italicized nucleotides in SEQ ID NO: 3 replaced by nucleotides complementary to those in SEQ ID NOs: 19-360 As used herein, “targets” means an operative RNA polynucleotide capable of undergoing hybridization to a nucleotide sequence through hydrogen bonding, such as to a nucleotide sequence transcribed from a nucleotide sequence within the larger genomic sequence of SNORD115. The hybridization of an operative RNA polynucleotide to a nucleotide sequence transcribed from a nucleotide sequence with the larger genomic sequence of SNORD115 may result in the reduced expression of SNORD115 and/or UBE3A-ATS levels in the presence of the operative RNA polynucleotide compared to the expression levels of SNORD115 and/or UBE3A-ATS in the absence of the operative RNA polynucleotide. In embodiments, the operative RNA polynucleotide encompasses the nucleotide sequence of the shRNA that is complementary to the RNA sequence encoded within the larger genomic sequence of SNORD115. For example, the shRNA contains nucleotide sequences complementary to the RNA sequences encoded by SEQ ID NOs: 19-360. The operative RNA polynucleotide thus refers to an operative portion of the shRNA following assimilation of the shRNA into a target organism and processing into a functional state.

“Reduce expression” refers to a reduction or blockade of the expression or activity of SNORD115 and/or UBE3A-ATS and does not necessarily indicate a total elimination of expression or activity. Mechanisms for reduced expression of the target include hybridization of an operative RNA polynucleotide with a target sequence or sequences transcribed from a sequence or sequences within the larger genomic SNORD115 and/or UBE3A-ATS sequence, wherein the outcome or effect of the hybridization is either target degradation or target occupancy with concomitant stalling of the cellular machinery involving, for example, transcription or splicing.

Without wishing to be bound to a particular theory, the shRNA herein may inhibit the silencing of paternal UBE3A by: (1) cutting the RNA transcript encoded by SEQ ID NO: 2; (2) reducing steady-state levels (i.e., baseline levels at homeostasis) of the RNA transcript encoded by SEQ ID NO: 2; (3) reducing steady-state levels (i.e., baseline levels at homeostasis) of the RNA transcript encoded by SEQ ID NO: 1; (4) terminating transcription of SEQ ID NO: 2, and (5) terminating transcription of SEQ ID NO: 1. For example, cutting and reduction of steady-state levels of the RNA transcript encoded by SEQ ID NO: 2 may occur via a mechanism involving a RNA-induced silencing complex (RISC). shRNA may utilize RISC. Once the vector carrying the genomic material for the shRNA is integrated into the host genome, the shRNA genomic material is transcribed in the host into pri-microRNA. The pri-microRNA is processed by a ribonuclease, such as Drosha, into pre-shRNA and exported from the nucleus. The pre-shRNA is processed by an endoribonuclease such as Dicer to form small interfering RNA (siRNA). The siRNA is loaded into the RISC where the sense strand is degraded and the antisense strand acts as a guide that directs RISC to the complementary sequence in the mRNA. RISC cleaves the mRNA when the sequence has perfect complementary and represses translation of the mRNA when the sequence has imperfect complementary. Thus, the shRNA encoded by the first nucleic acid sequence increases expression of paternal UBE3A by decreasing the steady-state levels of SNORD115 and/or UBE3A-ATS RNA.

As used herein, the term “nucleic acid” refers to molecules composed of monomeric nucleotides. Examples of nucleic acids include ribonucleic acids (RNA), deoxyribonucleic acids (DNA), single-stranded nucleic acids, double-stranded nucleic acids, small interfering ribonucleic acids (siRNA), and short hairpin RNAs (shRNAs) and ASOs. “Nucleotide” means a nucleoside having a phosphate group covalently linked to the sugar portion of the nucleoside. “Oligonucleotide” or “polynucleotide” means a polymer of linked nucleotides each of which can be modified or unmodified, independent one from another.

As used herein, a “short hairpin RNA (shRNA)” includes a conventional stem-loop shRNA, which forms a precursor microRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA (i.e., not a miR-451 shRNA mimic) forms a primary miRNA (pri-miRNA) or a structure very similar to a natural pri-miRNA. The pri-miRNA is subsequently processed by Drosha and its cofactors into pre-shRNA. Therefore, the term “shRNA” includes pri-miRNA (shRNA-mir) molecules and pre-shRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). It is known in the art that the loop portion is at least 4 nucleotides long, 6 nucleotides long (e.g., the underlined sequence in SEQ ID NO: 3), 8 nucleotides long, or more. The terms “hairpin” and “fold-back” structures are also used herein to refer to stem-loop structures. Such structures are well known in the art and the term is used consistently with its known meaning in the art. For example, CTCGAG (SEQ ID NO: 361), TCAAGAG (SEQ ID NO: 362), TTCG (SEQ ID NO: 363), and GAAGCTTG (SEQ ID NO: 364) are suitable stem-loop structures. As is known in the art, the secondary structure does not require exact base-pairing. Thus, the stem can include one or more base mismatches or bulges. Alternatively, the base-pairing can be exact, i.e., not include any mismatches. In embodiments, a polynucleotide sequence is provided as follows:
5′-TGATGATGAGAACCTTATATTnnnnnnnnAA TATAAGGTTCTCATCATCA-3′ (SEQ ID NO: 361), wherein nnnnnnnn can be CTCGAG (SEQ ID NO: 362), TCAAGAG (SEQ ID NO: 363), TTCG (SEQ ID NO: 364) or GAAGCTTG (SEQ ID NO: 365). In embodiments, a polynucleotide sequence is provided which includes a first portion, a second portion and a third portion, the first portion comprising any of SEQ ID NOs: 19-360, the second portion comprising any of SEQ ID Nos: 361, 362, 363, 364, and the third portion comprising respective nucleotide sequences complementary to those in SEQ ID NOs: 19-360.

In embodiments, shRNAs can include, without limitation, modified shRNAs, including shRNAs with enhanced stability in vivo. Modified shRNAs include molecules containing nucleotide analogues, including those molecules having additions, deletions, and/or substitutions in the nucleobase, sugar, or backbone; and molecules that are cross-linked or otherwise chemically modified. The modified nucleotide(s) may be within portions of the shRNA molecule, or throughout it. For instance, the shRNA molecule may be modified, or contain modified nucleic acids in regions at its 5′ end, its 3′ end, or both, and/or within the guide strand, passenger strand, or both, and/or within nucleotides that overhang the 5′ end, the 3′ end, or both. (See Crooke, U.S. Pat. Nos. 6,107,094 and 5,898,031; Elmen et al., U.S. Publication Nos. 2008/0249039 and 2007/0191294; Manoharan et al., U.S. Publication No. 2008/0213891; MacLachlan et al., U.S. Publication No. 2007/0135372; and Rana, U.S. Publication No. 2005/0020521; all of which are hereby incorporated by reference.)

shRNAs herein include a nucleotide sequence complementary to a RNA nucleotide sequence transcribed from within the full genomic SNORD115 sequence (SEQ ID NO: 2) and inhibit the silencing of paternal UBE3A by UBE3A-ATS. In embodiments, shRNAs include a nucleotide sequence complementary to RNA sequences encoded by SEQ ID NOs: 19-360. In embodiments, a shRNA includes a nucleotide sequence complementary to a RNA sequence encoded by SEQ ID NO: 21 (5′-TGATGATGAGAACCTTATATT-3′). In embodiments, the shRNA is encoded by the nucleotide sequence of SEQ ID NO: 2. In embodiments, the nucleotide sequence included in the shRNA and complementary to the RNA nucleotide sequence transcribed from the SNORD115 gene is 17-21 nucleotides in length. The complementary nucleotides may be contiguous or may be interspersed with non-complementary nucleotides. In embodiments, the complementary nucleotide sequence is 21 nucleotides in length as indicated by the bold sequence in SEQ ID NO: 3. The shRNA may include a nucleotide sequence wherein 17, 18, 19, 20, or 21 nucleotides are complementary to the nucleotides in SEQ ID NOs: 19-360. The 17, 18, 19, 20, or 21 complementary nucleotides may be contiguous or may be interspersed with non-complementary nucleotides. The overall length of the shRNA, including the loop may be 40-50 nucleotides in length, e.g., 44-48 nucleotides, e.g., 48 nucleotides.

Methods of determining whether a sequence is specifically hybridizable to a target nucleic acid are well known in the art. In embodiments, the shRNA polynucleotides provided herein include a nucleic acid sequence specifically hybridizable with a RNA sequence transcribed from the SNORD115 (SEQ ID NO: 2).

The shRNA may include an RNA polynucleotide containing a region of 17-21 linked nucleotides complementary to the RNA target sequence, wherein the RNA polynucleotide region is at least 85% complementary over its entire length to an equal length region of a SNORD115 RNA nucleic acid sequence. In embodiments, the 17-21 RNA polynucleotide region is at least 90%, at least 95%, or 100% complementary over its entire length to an equal length region of a SNORD115 RNA nucleic acid sequence, e.g., encoded by SEQ ID NOs: 4-18.

The shRNA may include a nucleotide sequence at least 85% complementary to, and of equal length as, a RNA sequence encoded by any of SEQ ID NOs: 19-360, e.g., SEQ ID NO: 21. The shRNA may include a nucleotide sequence at least 90% complementary to, and of equal length as, a RNA sequence encoded by any of SEQ ID NOs: 19-360. The shRNA may include a nucleotide at least 95% complementary to, and of equal length as, a RNA sequence encoded by any of SEQ ID NOs: 19-360. The shRNA or microRNA may encompass a nucleotide sequence 100% complementary to, and of equal length as, a RNA sequence encoded by any of SEQ ID NOs: 19-360.

In embodiments, the shRNA is a single-stranded RNA polynucleotide. In embodiments, the RNA polynucleotide is a modified RNA polynucleotide. A percent complementarity is used herein in the conventional sense to refer to base pairing between adenine and thymine, adenine and uracil (RNA), and guanine and cytosine.

Non-complementary nucleobases between a shRNA and a SNORD115 nucleotide sequence may be tolerated provided that the shRNA remains able to specifically hybridize to a SNORD115 nucleotide sequence. Moreover, a shRNA may hybridize over one or more segments of a SNORD115 nucleotide sequence such that intervening or adjacent segments are not involved in the hybridization event (e.g., a loop structure, mismatch or hairpin structure).

In embodiments, the shRNA provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a SNORD115 RNA nucleotide sequence, a SNORD115 region, SNORD115 segment, or specified portion thereof. Percent complementarity of a shRNA with an SNORD115 nucleotide sequence can be determined using routine methods.

For example, a shRNA in which 18 of 20 nucleobases are complementary to a SNORD115 region and would therefore specifically hybridize, would represent 90 percent complementarity. In this example, the remaining non-complementary nucleobases may be clustered or interspersed with complementary nucleobases and need not be contiguous to each other or to complementary nucleobases. As such, a shRNA which is 18 nucleobases in length having four non-complementary nucleobases which are flanked by two regions of complete complementarity with the target nucleotide sequence would have 77.8% overall complementarity with the target nucleotide sequence and would thus fall within the subject matter disclosed herein. Percent complementarity of a shRNA with a region of a SNORD115 nucleotide sequence can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In embodiments, the shRNA provided herein, or specified portions thereof, are fully complementary (i.e., 100% complementary) to a SNORD115 nucleotide sequence, or specified portion of the transcription product of SEQ ID NO: 1 thereof. For example, a shRNA may be fully complementary to a SNORD115 nucleotide sequence, or a region, or a segment or sequence thereof. As used herein, “fully complementary” means each nucleobase of a shRNA is capable of precise base pairing with the corresponding RNA nucleobases transcribed from a SNORD115 nucleotide sequence.

In embodiments, the shRNA provided herein can contain a portion of SEQ ID NO: 3, e.g., having the bold nucleotides, which has been shortened by one, two, three or four nucleotides at either end of the bold nucleotides. Likewise, in embodiments, the shRNA provided herein can contain a portion of SEQ ID NO: 3, e.g., having the italicized nucleotides, which has been shortened by one, two, three or four nucleotides at either end of the italicized nucleotides. For example,

(SEQ ID NO: 366)
5′-GATGATGAGAACCTTATATTCTCGAGAATATAAGGTTCTCATCATC-3′
(SEQ ID NO: 367)
5′-ATGATGAGAACCTTATATTCTCGAGAATATAAGGTTCTCATCAT-3′
(SEQ ID NO: 368)
5′-TGATGAGAACCTTATATTCTCGAGAATATAAGGTTCTCATCA-3′
(SEQ ID NO: 369)
5′-GATGAGAACCTTATATTCTCGAGAATATAAGGTTCTCATC-3′
(SEQ ID NO: 370)
5′-TGATGATGAGAACCTTATATCTCGAGATATAAGGTTCTCATCATCA-3′
(SEQ ID NO: 371)
5′-TGATGATGAGAACCTTATACTCGAGTATAAGGTTCTCATCATCA-3′
(SEQ ID NO: 372)
5′-TGATGATGAGAACCTTATCTCGAGATAAGGTTCTCATCATCA-3′
(SEQ ID NO: 373)
5′-TGATGATGAGAACCTTACTCGAGTAAGGTTCTCATCATCA-3′
(SEQ ID NO: 374)
5′-GATGATGAGAACCTTATATTnnnnnnnnAATATAAGGTTCTCATCATC-3′,
(SEQ ID NO: 375)
5′-ATGATGAGAACCTTATATTnnnnnnnnAATATAAGGTTCTCATCAT-3′
(SEQ ID NO: 376)
5′-TGATGAGAACCTTATATTnnnnnnnnAATATAAGGTTCTCATCA-3′
(SEQ ID NO: 377)
5′-GATGAGAACCTTATATTnnnnnnnnAATATAAGGTTCTCATC-3′
(SEQ ID NO: 378)
5′-TGATGATGAGAACCTTATATnnnnnnnnATATAAGGTTCTCATCATCA-3′
(SEQ ID NO: 379)
5′-TGATGATGAGAACCTTATAnnnnnnnnTATAAGGTTCTCATCATCA-3′
(SEQ ID NO: 380)
5′-TGATGATGAGAACCTTATnnnnnnnnATAAGGTTCTCATCATCA-3′
(SEQ ID NO: 381)
5′-TGATGATGAGAACCTTAnnnnnnnnTAAGGTTCTCATCATCA-3′, wherein
nnnnnnnn for any of the above SEQ ID NOs: 374-365 can be
(SEQ ID NO: 362)
CTCGAG,
(SEQ ID NO: 363)
TCAAGAG,
(SEQ ID NO: 364)
TTCG
or
(SEQ ID NO: 365)
GAAGCTTG.

Similarly, in embodiments, the sequences shown in any of SEQ ID NOs: 19-360 and/or their complements can be shortened by one, two, three or four nucleotides at either end and incorporated into shRNAs as shown, for example, above.

An effective concentration or dose of the shRNA may inhibit the silencing of paternal UBE3A by UBE3A-ATS by at least 20%, at least 30%, at least 40%, at least 50%, at least 60%, at least 70%, at least 80%, at least 90%, or at least 100%.

An effective concentration or dose of the shRNA may terminate transcription of UBE3A-ATS by at least 20%, at least 30%, at least 40%, at least 50%, at least 60%, at least 70%, at least 80%, at least 90%, or at least 100%.

An effective concentration or dose of the shRNA may reduce steady-state levels of UBE3A-ATS by at least 20%, at least 30%, at least 40%, at least 50%, at least 60%, at least 70%, at least 80%, at least 90%, or at least 100%.

An effective concentration or dose of the shRNA cuts SNORD115 and reduces it by at least 20%, at least 30%, at least 40%, at least 50%, at least 60%, at least 70%, at least 80%, at least 90%, or at least 100%.

An effective concentration or dose of the shRNA may reduce expression of UBE3A-ATS by at least 20%, at least 30%, at least 40%, at least 50%, at least 60%, at least 70%, at least 80%, at least 90%, or at least 100% and induce expression of paternal UBE3A by at least 20%, at least 30%, at least 40%, at least 50%, at least 60%, at least 70%, at least 80%, at least 90%, or at least 100%.

As used herein, the terms “UBE3A-ATS” and “Ube3A-ATS” can be used interchangeably without capitalization of their spelling referring to any particular species or ortholog. “UBE3A” and “Ube3A” can be used interchangeably without capitalization of their spelling referring to any particular species or ortholog. Additionally, “UBE3A”, “UBE3A”, “Ube3A”, and “Ube3A” can be used interchangeably without italicization referring to nucleic acid or protein unless specifically indicated to the contrary. “SNORD115” and “SNORD115” can be used interchangeably without capitalization of their spelling referring to any particular species or ortholog.

Viral Vector

A “vector” is a replicon, such as a plasmid, phage, or cosmid, into which a DNA segment or an RNA segment may be inserted so as to bring about the replication of the inserted segment. Generally, a vector is capable of replication when associated with the proper control elements. Suitable vector backbones include, for example, those routinely used in the art such as plasmids, plasmids that contain a viral genome, viruses, or artificial chromosomes. The term “vector” includes cloning and expression vectors, as well as viral vectors and integrating vectors.

As will be evident to one of skill in the art, the term “viral vector” is widely used to refer to a nucleic acid molecule (e.g., a transfer plasmid) that includes viral nucleic acid elements that typically facilitate transfer of the nucleic acid molecule to a cell or to a viral particle that mediates nucleic acid sequence transfer and/or integration of the nucleic acid sequence into the genome of a cell.

Viral vectors contain structural and/or functional genetic elements that are primarily derived from a virus. The viral vector is desirably non-toxic, non-immunogenic, easy to produce, and efficient in protecting and delivering DNA or RNA into the target cells. According to the compositions and methods described herein a viral vector may contain the DNA that encodes one or more of the shRNAs described herein. In embodiments, the viral vector is a lentiviral vector or an adeno-associated viral (AAV) vector.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). As used herein, the term “lentivirus” includes lentivirus particles. Lentivirus will transduce dividing cells and postmitotic cells.

The term “lentiviral vector” refers to a viral vector (e.g., viral plasmid) containing structural and functional genetic elements, or portions thereof, including long terminal repeats (LTRs) that are primarily derived from a lentivirus. A lentiviral vector is a hybrid vector (e.g., in the form of a transfer plasmid) having retroviral, e.g., lentiviral, sequences for reverse transcription, replication, integration and/or packaging of nucleic acid sequences (e.g., coding sequences). The term “retroviral vector” refers to a viral vector (e.g., transfer plasmid) containing structural and functional genetic elements, or portions thereof, that are primarily derived from a retrovirus.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and non-dividing cells in many different tissues in vivo including airway epithelial cells, endothelial cells, hepatocytes, and various tumors (Trapnell, Advanced Drug Delivery, Reviews, 12 (1993) 185-199).

The term “adeno-associated virus” (AAV) refers to a small ssDNA virus which infects humans and some other primate species, not known to cause disease, and causes only a very mild immune response. As used herein, the term “AAV” is meant to include AAV particles. AAV can infect both dividing and non-dividing cells and may incorporate its genome into that of the host cell. These features make AAV an attractive candidate for creating viral vectors for gene therapy, although the cloning capacity of the vector is relatively limited. In embodiments, the vector used is derived from adeno-associated virus (i.e., AAV vector). More than 30 naturally occurring serotypes of AAV are available. Many natural variants in the AAV capsid exist, allowing identification and use of an AAV with properties specifically suited for specific types of target cells. AAV viruses may be engineered by conventional molecular biology techniques, making it possible to optimize these particles for cell specific delivery of shRNA DNA sequences, for minimizing immunogenicity, for tuning stability and particle lifetime, for efficient degradation, for accurate delivery to the nucleus, etc.

An “expression vector” is a vector that includes a regulatory region. Numerous vectors and expression systems are commercially available from such corporations as Novagen (Madison, Wis.), Clontech (Palo Alto, Calif), Stratagene (La Jolla, Calif.), and Invitrogen/Life Technologies (Carlsbad, Calif). An expression vector may be a viral expression vector derived from a particular virus.

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FIag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of pLK0.1 puro, SV40 and, plasmids such as RP4; phage DNAs, e.g., the numerous derivatives of phage 1, e.g., NM989, and other phage DNA, e.g., M13 and filamentous single stranded phage DNA, vectors useful in eukaryotic cells, such as vectors useful in insect or mammalian cells, vectors derived from combinations of plasmids and phage DNAs, such as plasmids that have been modified to employ phage DNA or other expression control sequences.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically includes at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. Modulation of the expression of a coding sequence can be accomplished by appropriately selecting and positioning promoters and other regulatory regions relative to the coding sequence.

Vectors can also include other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector including one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation).

In embodiments, the viral vector used herein will be used, e.g., at a concentration of at least 10⁵ viral genomes per cell.

The selection of appropriate promoters can readily be accomplished. Examples of suitable promoters include RNA polymerase II or III promoters. For example, candidate shRNA sequences may be expressed under control of RNA polymerase III promoters U6 or H1, or neuron-specific RNA polymerase II promoters including neuron-specific enolase (NSE), synapsin I (Syn), or the Ca2+/CaM-activated protein kinase II alpha (CaMKIIalpha).

Other suitable promoters which may be used for gene expression include, but are not limited to, the 763-base-pair cytomegalovirus (CMV) promoter, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, PGK (phosphoglycerol kinase) promoter, alkaline phosphatase promoter; and the animal transcriptional control regions, which exhibit tissue specificity and have been utilized in transgenic animals: myelin basic protein gene control region which is active in oligodendrocyte cells in the brain, and gonadotropic releasing hormone gene control region which is active in the hypothalamus. Certain proteins can be expressed using their native promoter. Other elements that can enhance expression can also be included such as an enhancer or a system that results in high levels of expression such as a tat gene and tar element. The assembly or cassette can then be inserted into a vector, e.g., a plasmid vector such as, pLK0.1, pUC19, pUC118, pBR322, or other known plasmid vectors. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

Coding sequences for shRNA can be cloned into viral vectors using any suitable genetic engineering technique well known in the art, including, without limitation, the standard techniques of PCR, polynucleotide synthesis, restriction endonuclease digestion, ligation, transformation, plasmid purification, and DNA sequencing, as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)). In embodiments, the shRNA DNA sequences contain flanking sequences on the 5′ and 3′ ends that are complementary with sequences on the plasmid and/or vector that is cut by a restriction endonuclease. As is well known in the art, the flanking sequences depend on the restriction endonucleases used during the restriction digest of the plasmid and/or vector. Thus, one of skill in the art can select the flanking sequences on the 5′ and 3′ ends of the shRNA DNA sequences accordingly. In embodiments, the target sites can be cloned into vectors by nucleic acid fusion and exchange technologies currently known in the art, including, Gateway, PCR in fusion, Cre-lox P, and Creator.

In embodiments, an expression vector includes a promoter and a polynucleotide including a first nucleotide sequence encoding a shRNA described herein. In embodiments, the promoter and the polynucleotide including the first nucleotide sequence are operably linked. In embodiments, the promoter is a U6 promoter. In embodiments, the first nucleotide sequence included in the expression vector may be SEQ ID NO: 3. In embodiments, the first nucleotide sequence included in the expression vector may also be a modified SEQ ID NO: 3 having the bold nucleotides in SEQ ID NO: 3 replaced by any of SEQ ID NOs: 19-360 and the italicized nucleotides in SEQ ID NO: 3 replaced by nucleotides complementary to those in SEQ ID NOs: 19-360. In embodiments, the first nucleotide sequence included in the expression vector may include any of SEQ ID Nos: 362-365. In embodiments, the first nucleotide sequence included in the expression vector may include any of SEQ ID Nos: 361-381. In embodiments, the polynucleotide including the first nucleotide sequence in the expression vector is a DNA polynucleotide. In embodiments, the first nucleotide sequence of the expression vector is a DNA nucleotide sequence. The shRNA encoded by the first nucleotide sequence of the expression vector may be as described in any of the variations disclosed herein.

As discussed below, recombinant viral vectors are transfected into packaging cells or cell lines, along with elements required for the packaging of recombinant viral particles. Recombinant viral particles collected from transfected cell supernatant are used to infect target cells or organisms for the expression of shRNAs. The transduced cells or organisms are used for transient expression or selected for stable expression.

Virus/Viral Particle

Viral particles are used to deliver coding nucleotide sequences for the shRNAs which target SNORD115 RNA. The terms virus and viral particles are used interchangeably herein. Viral particles will typically include various viral components and sometimes also host cell components in addition to nucleic acid(s). Nucleic acid sequences may be packaged into a viral particle that is capable of delivering the shRNA nucleic acid sequences into the target cells in the patient in need.

The viral particles may be produced by (a) introducing a viral expression vector into a suitable cell line; (b) culturing the cell line under suitable conditions so as to allow the production of the viral particle; (c) recovering the produced viral particle; and (d) optionally purifying the recovered infectious viral particle.

An expression vector containing the nucleotide sequence encoding one or more of the shRNAs herein may be introduced into an appropriate cell line for propagation or expression using well-known techniques readily available to the person of ordinary skill in the art. These include, but are not limited to, microinjection of minute amounts of DNA into the nucleus of a cell (Capechi et al., 1980, Cell 22, 479-488), CaPO₄-mediated transfection (Chen and Okayama, 1987, Mol. Cell Biol. 7, 2745-2752), DEAE-dextran-mediated transfection, electroporation (Chu et al., 1987, Nucleic Acid Res. 15, 1311-1326), lipofection/liposome fusion (Feigner et al., 1987, Proc. Natl. Acad. Sci. USA 84, 7413-7417), particle bombardment (Yang et al., 1990, Proc. Natl. Acad. Sci. USA 87, 9568-9572), gene guns, transduction, infection (e.g. with an infective viral particle), and other techniques such as those found in Sambrook, et al. (Molecular Cloning: A Laboratory Manual, Cold Spring Harbor Laboratory, Cold Spring Harbor Laboratory Press, Cold Spring Harbor, N.Y., 2001).

In embodiments, where an expression vector is defective, infectious particles can be produced in a complementation cell line or via the use of a helper virus, which supplies in trans the non-functional viral genes. For example, suitable cell lines for complementing adenoviral vectors include the 293 cells (Graham et al., 1997, J. Gen. Virol. 36, 59-72) as well as the PER-C6 cells (Fallaux et al., 1998, Human Gene Ther. 9, 1909-1917) commonly used to complement the E1 function. Other cell lines have been engineered to complement doubly defective adenoviral vectors (Yeh et al., 1996, J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033; WO94/28152 and WO97/04119). The infectious viral particles may be recovered from the culture supernatant but also from the cells after lysis and optionally are further purified according to standard techniques (chromatography, ultracentrifugation in a cesium chloride gradient as described for example in WO 96/27677, WO 98/00524, WO 98/22588, WO 98/26048, WO 00/40702, EP 1016700 and WO 00/50573).

In embodiments, provided herein are host cells which include the nucleic acid molecules, vectors, or infectious viral particles described herein. The term “host cell” should be understood broadly without any limitation concerning particular organization in tissue, organ, or isolated cells. Such cells may be of a unique type of cells or a group of different types of cells and encompass cultured cell lines, primary cells, and proliferative cells.

Host cells therefore include prokaryotic cells, lower eukaryotic cells such as yeast, and other eukaryotic cells such as insect cells, plant and higher eukaryotic cells, such as vertebrate cells and, with a special preference, mammalian (e.g., human or non-human) cells. Suitable mammalian cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle) or fibroblasts. For example, host cells can include Escherichia coli, Bacillus, Listeria, Saccharomyces, BHK (baby hamster kidney) cells, MDCK cells (Madin-Darby canine kidney cell line), CRFK cells (Crandell feline kidney cell line), CV-1 cells (African monkey kidney cell line), COS (e.g., COS-7) cells, chinese hamster ovary (CHO) cells, mouse NIH/3T3 cells, HeLa cells and Vero cells. Host cells also encompass complementing cells capable of complementing at least one defective function of a replication-defective vector utilizable herein (e.g., a defective adenoviral vector) such as those cited above.

In embodiments, the host cell may be encapsulated. Cell encapsulation technology has been previously described (Tresco et al., 1992, ASAJO J. 38, 17-23; Aebischer et al., 1996, Human Gene Ther. 7, 851-860). For example, transfected or infected eukaryotic host cells can be encapsulated with compounds which form a microporous membrane and said encapsulated cells may further be implanted in vivo. Capsules containing the cells of interest may be prepared employing hollow microporous membranes (e.g. Akzo Nobel Faser AG, Wuppertal, Germany; Deglon et al. 1996, Human Gene Ther. 7, 2135-2146) having a molecular weight cutoff appropriate to permit the free passage of proteins and nutrients between the capsule interior and exterior, while preventing the contact of transplanted cells with host cells

Viral particles suitable for use herein include AAV particles and lentiviral particles. AAV particles carry the coding sequences for shRNAs herein in the form of genomic DNA. Lentiviral particles, on the other hand, belong to the class of retroviruses and carry the coding sequences for shRNAs herein in the form of RNA.

Recombinantly engineered viral particles such as AAV particles, artificial AAV particles, self-complementary AAV particles, and lentiviral particles that contain the DNA (or RNA in the case of lentiviral particles) encoding the shRNAs targeting SNORD115 RNA may be delivered to target cells to inhibit the silencing of UBE3A by UBE3A-ATS. The use of AAVs is a common mode of delivery of DNA as it is relatively non-toxic, provides efficient gene transfer, and can be easily optimized for specific purposes. In embodiments, the selected AAV serotype has native neurotropisms. In embodiments, the AAV serotype is AAV9 or AAV10.

A suitable recombinant AAV can be generated by culturing a host cell which contains a nucleotide sequence encoding an AAV serotype capsid protein, or fragment thereof, as defined herein; a functional rep gene; a minigene composed of, at a minimum, AAV inverted terminal repeats (ITRs) and a coding nucleotide sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Unless otherwise specified, the AAV inverted terminal repeats (ITRs), and other selected AAV components described herein, may be readily selected from among any AAV serotype, including, without limitation, AAV1, AAV2, AAV3, AAV5, AAV6, AAV7, AAV8, AAV9, AAV10, AAVRec3 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene, rep sequences, cap sequences, and helper functions required for producing a rAAV herein may be delivered to the packaging host cell in the form of any genetic element which transfers the sequences carried thereon. The selected genetic element may be delivered by any suitable method. The methods used to construct embodiments herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation. See, e.g., K. Fisher et al, 1993 J. Viral., 70:520-532 and U.S. Pat. No. 5,478,745, among others. All citations herein are incorporated by reference herein.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes herein. However, one of skill in the art may make a selection among these, and other, expression control sequences.

Pharmaceutical Compositions and Therapeutic Treatment

The virus including the desired coding sequences for the shRNA, can be formulated for administration to a patient or human in need by any means suitable for administration. Such formulation involves the use of a pharmaceutically and/or physiologically acceptable vehicle or carrier, particularly one suitable for administration to the brain, e.g., by subcranial or spinal injection. Further, more than one shRNA herein may be administered in a combination treatment. In a combination treatment, the different shRNAs may be administered simultaneously, separately, sequentially, and in any order.

Pharmaceutical compositions herein include a carrier and/or diluent appropriate for its delivering by injection to a human or animal organism. Such carrier and/or diluent should be generally non-toxic at the dosage and concentration employed. It can be selected from those usually employed to formulate compositions for parental administration in either unit dosage or multi-dose form or for direct infusion by continuous or periodic infusion. In embodiments, it is isotonic, hypotonic or weakly hypertonic and has a relatively low ionic strength, such as provided by sugars, polyalcohols and isotonic saline solutions. Representative examples include sterile water, physiological saline (e.g., sodium chloride), bacteriostatic water, Ringer's solution, glucose or saccharose solutions, Hank's solution, and other aqueous physiologically balanced salt solutions (see for example the most current edition of Remington: The Science and Practice of Pharmacy, A. Gennaro, Lippincott, Williams & Wilkins). The pH of the composition is suitably adjusted and buffered in order to be appropriate for use in humans or animals, e.g., at a physiological or slightly basic pH (between about pH 8 to about pH 9, with a special preference for pH 8.5). Suitable buffers include phosphate buffer (e.g., PBS), bicarbonate buffer and/or Tris buffer. In embodiments, e.g., a composition is formulated in 1M saccharose, 150 mM NaCl, 1 mM MgCl₂, 54 mg/l Tween 80, 10 mM Tris pH 8.5. In embodiments, e.g., a composition is formulated in 10 mg/ml mannitol, 1 mg/ml HSA, 20 mM Tris, pH 7.2, and 150 mM NaCl. These compositions are stable at −70° C. for at least six months.

Pharmaceutical compositions herein may be in various forms, e.g., in solid (e.g. powder, lyophilized form), or liquid (e.g. aqueous). In the case of solid compositions, methods of preparation are, e.g., vacuum drying and freeze-drying which yields a powder of the active agent plus any additional desired ingredient from a previously sterile-filtered solution thereof. Such solutions can, if desired, be stored in a sterile ampoule ready for reconstitution by the addition of sterile water for ready injection.

Nebulized or aerosolized formulations are also suitable. Methods of intranasal administration are well known in the art, including the administration of a droplet, spray, or dry powdered form of the composition into the nasopharynx of the individual to be treated from a pressured container or dispenser which contains a suitable propellant, e.g., a gas such as carbon dioxide, or a nebulizer (see for example WO 95/11664). Enteric formulations such as gastroresistant capsules and granules for oral administration, suppositories for rectal or vaginal administration may also be suitable. For non-parental administration, the compositions can also include absorption enhancers which increase the pore size of the mucosal membrane. Such absorption enhancers include sodium deoxycholate, sodium glycocholate, dimethyl-beta-cyclodextrin, lauroyl-1-lysophosphatidylcholine and other substances having structural similarities to the phospholipid domains of the mucosal membrane.

The composition can also contain other pharmaceutically acceptable excipients for providing desirable pharmaceutical or pharmacodynamic properties, including for example modifying or maintaining the pH, osmolarity, viscosity, clarity, color, sterility, stability, rate of dissolution of the formulation, modifying or maintaining release or absorption into an the human or animal organism. For example, polymers such as polyethylene glycol may be used to obtain desirable properties of solubility, stability, half-life and other pharmaceutically advantageous properties (Davis et al., 1978, Enzyme Eng. 4, 169-173; Burnham et al., 1994, Am. J. Hosp. Pharm. 51, 210-218). Representative examples of stabilizing components include polysorbate 80, L-arginine, polyvinylpyrrolidone, trehalose, and combinations thereof. Other stabilizing components especially suitable in plasmid-based compositions include hyaluronidase (which is thought to destabilize the extra cellular matrix of the host cells as described in WO 98/53853), chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile (see EP 890 362), nuclease inhibitors such as actin G (WO 99/56784) and cationic salts such as magnesium (Mg′) (EP 998 945) and lithium (Lit) (WO 01/47563) and any of their derivatives. The amount of cationic salt in the composition herein preferably ranges from about 0.1 mM to about 100 mM, and still more preferably from about 0.1 mM to about 10 mM. Viscosity enhancing agents include sodium carboxymethylcellulose, sorbitol, and dextran. The composition can also contain substances known in the art to promote penetration or transport across the blood barrier or membrane of a particular organ (e.g., antibody to transferrin receptor; Friden et al., 1993, Science 259, 373-377). A gel complex of poly-lysine and lactose (Midoux et al., 1993, Nucleic Acid Res. 21, 871-878) or poloxamer 407 (Pastore, 1994, Circulation 90, 1-517) may be used to facilitate administration in arterial cells.

The viral particles and pharmaceutical compositions may be administered to patients in therapeutically effective amounts. As used herein, the term “therapeutically effective amount” refers to an amount sufficient to realize a desired biological effect. For example, a therapeutically effective amount for treating Angelman's syndrome is an amount sufficient to ameliorate one or more symptoms of Angelman's syndrome, as described herein (e.g., developmental delay, severe cognitive impairment, ataxic gait, frequent seizures, short attention span, absent speech, and characteristic happy demeanor). Further, AS iPSC-derived neurons or AS hESC derived neurons exhibit a depolarized resting membrane potential, delayed action potential development, and reduced spontaneous synaptic activity. Thus, a therapeutically effective amount for treating AS may return the neuronal resting membrane potential to about −70 mV, ameliorate the action potential development delay, increase spontaneous synaptic activity, or ameliorate additional alterations in the neuronal phenotype relating to rheobase, action potential characteristics (e.g., shape), membrane current, synaptic potentials, ion channel conductance, etc.

The appropriate dosage may vary depending upon known factors such as the pharmacodynamic characteristics of the particular active agent, age, health, and weight of the host organism; the condition(s) to be treated, nature and extent of symptoms, kind of concurrent treatment, frequency of treatment, the need for prevention or therapy and/or the effect desired. The dosage will also be calculated dependent upon the particular route of administration selected. Further refinement of the calculations necessary to determine the appropriate dosage for treatment can be made by a practitioner, in the light of the relevant circumstances. For general guidance, a composition based on viral particles may be formulated in the form of doses of, e.g., at least 10⁵ viral genomes per cell. The titer may be determined by conventional techniques. A composition based on vector plasmids may be formulated in the form of doses of between 1 μg to 100 mg, e.g., between 10 μg and 10 mg, e.g., between 100 μg and 1 mg. The administration may take place in a single dose or a dose repeated one or several times after a certain time interval.

Pharmaceutical compositions herein can be enclosed in ampoules, disposable syringes or multiple dose vials made of glass or plastic. In all cases, the composition should be sterile and should be fluid to the extent that easy syringability exists. It should be stable under the conditions of manufacture and storage and preserved against the contaminating action of microorganisms such as bacteria and fungi. Sterile injectable solutions can be prepared by incorporating the active agent (e.g., infectious particles) in the required amount with one or a combination of ingredients enumerated above, followed by filtered sterilization.

The viral particles and pharmaceutical compositions herein may be administered by a parenteral route including intravenous, intraarterial, subcutaneous, intraperitoneal or intramuscular injection or infusion, intrathecal or intracranial, e.g., intracerebral or intraventricular, administration. In embodiments, viral particles or pharmaceutical compositions are administered intracerebrally or intracerebroventricularly. In embodiments, the viral particles or pharmaceutical compositions herein are administered intrathecally.

In embodiments, the viral particles and a pharmaceutical composition described above are administered to the subject by subcranial injection into the brain or into the spinal cord of the patient or human in need. In embodiments, the use of subcranial administration into the brain results in the administration of the encoding nucleotide sequences described herein directly to brain cells, including glia and neurons. As used herein, the term “neuron” refers to any cell in, or associated with, the function of the brain. The term may refer to any one the types of neurons, including unipolar, bipolar, multipolar and pseudo-unipolar.

### Examples

shRNA Vector Generation and Lentiviral Preparation

Oligonucleotides encoding shRNAs were cloned into the pLKO.1-puro vector, which drives expression of the small RNA by the U6 promoter (Addgene plasmid #8453). Specifically, the polynucleotides to generate shRNAs encompassed the specific 21-nucleotide sequence to be targeted and its reverse complement, separated by a loop sequence of CTCGAG, and with a 5′ flank sequence of CCGG and a 3′ flank sequence of TTTTTG added for cloning into the plasmid vector. The following oligonucleotides encoding shRNAs as well as a scrambled shRNA control were utilized:

SNORD115 shRNA 1 (SEQ ID NO: 382):
(5′-CAATGATGAGAACCGTATATTCTCGAGAATATACGGTTCTCATCA
TTG-3′)
SNORD115 shRNA 2 (SEQ ID NO: 383):
(5′-GGTAACCTGTTCTCCAAATTTCTCGAGAAATTTGGAGAACAGGTT
ACC-3′)
SNORD115 shRNA 3 (SEQ ID NO: 3):
(5′-TGATGATGAGAACCTTATATTCTCGAGAATATAAGGTTCTCATCA
TCA-3′).

Cloning was verified by Sanger sequencing. Lentiviral particles were produced from cloned shRNAs in HEK293T cells using second generation lentiviral packaging plasmids (psPAX2, Addgene plasmid #12260; pMD2.G, Addgene plasmid #12259) and concentrated using the Lenti-X Concentrator Kit (Takara). Lentiviral titer was estimated using a qPCR kit detecting the 5′LTR (Applied Biological Materials).

Stem Cell Culture and Neuronal Differentiation

Angelman syndrome (AS) human embryonic stem cells (hESCs) were maintained under feeder-free conditions on Matrigel-coated substrates (Corning) in mTeSR-plus medium (Stem Cell Technologies). hESCs were cultured in at 37° C. in a humid incubator at 5% CO2. Cells were fed daily and passaged using 0.5 mM EDTA every four-five days. Glutamatergic neurons were generated from hESCs by doxycycline inducible expression of the human neurogenin2 (NGN2) transgene (Fernandopulle et al., 2018, Curr Protoc Cell Biol. 79(1): e51.). Briefly, the doxycycline-inducible NGN2 construct was stably integrated into the safe-harbor AAVS1 locus of AS hESCs using a pair of AAVS1 targeting TALENS and clonal cell lines were subsequently derived. Neuronal induction was then carried out by culturing these hESCs in Neural Induction Media consisting of DMEM/F12, N2 Supplement, Non-essential amino acids (NEAA), L-glutamine (all Gibco products), and 2 ug/mL doxycycline for three days. Neurons were then plated for terminal maturation in Cortical Neuron Medium consisting of DMEM/F12, Neurobasal Medium, B27 Supplement, Penicillin/Streptomycin (all Gibco products), BDNF (long/mL), GDNF (long/mL), NT-3 (long/mL), and Laminin (lug/mL). Human ESC-derived NGN2-induced neurons (7-10 days post-induction) were transduced with lentiviral particles at an MOI of 10.

Quantitative RT-PCR (qRT-PCR) Analysis

Neurons were collected for RNA isolation and qRT-PCR 7 days after viral transduction. Total RNA was isolated from hESC-derived neurons using RNA-STAT60 (AMS Biotechnology) according to the manufacturer's protocol. cDNA was produced using the High Capacity cDNA Reverse Transcription Kit (Life Technologies). Gene expression analysis was performed at least in triplicate. All qPCR assays used were TaqMan Gene Expression Assays (Life Technologies). Ct values for each gene were normalized to the house keeping gene GAPDH. Relative expression was quantified as 2^{-ΔΔCt} relative to the calibrator sample.

Data Summary and Results

AS hESC-derived neurons were transduced with lentiviral particles to express the selected shRNA sequences targeting the SNHG14 long non-coding RNA. qRT-PCR was used to determine the expression of UBE3A-ATS, the SNORD115 host gene, and UBE3A in SNHG14-shRNA-treated neurons relative to neurons treated with a non-targeting control shRNA (SCRAM). FIG. 4 reflects qRT-PCR analysis of AS hESC-derived neurons following treatment with either SNHG14-targeting shRNAs (SNORD115 shRNA 1, SNORD115 shRNA 2, SNORD115 shRNA 3) or non-targeting control shRNA (SCRAM). Expression of UBE3A-ATS, UBE3A, and SNORD115 were normalized to the housekeeping gene GAPDH, and expression is presented relative to SCRAM-shRNA treated neurons. Error bars represent standard error of the mean, n=3 biological replicates. As shown in FIG. 4, SNORD115 shRNA 3 effectively reduced RNA levels of UBE3A-ATS (40% reduction) and SNORD115 (55% reduction) compared to SCRAM controls. This reduction in SNHG14 transcript levels was associated with a robust increase in UBE3A RNA (2.8fold increase over SCRAM controls). Although SNORD115 shRNA 1 and SNORD115 shRNA 2 were predicted to reduce RNA levels of UBE3A-ATS and SNORD115 and increase UBE3A expression, they did not have that effect.

It is understood that the foregoing detailed description and accompanying examples are merely illustrative and are not to be taken as limitations upon the scope of the subject matter described herein, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use, may be made without departing from the spirit and scope thereof.

Sequences

```
SEQ ID NO: 1
Human UBE3A-ATS genomic sequence.
TGAGATGACCTAAACAACTGTGGAGAATCATTGATATATTTCCTTTTTTCACTGTTCATGTTGG
GTGAAAATAATCTTGTAGTGAAATTCACATGTTCTAAATATTGTTTTTTTACATCTTTATCTGG
CACATTCATAACATAGATGTTTCTATACATATTAGTACTGTAATCATACCATATATTATTCTGT
TACCCCACTTACTCCTTAAACTTTTAGTTAATTAAAGAGTTTTTATAAAGTCCCCCAATAGATT
TTTTTTTTTTGAGACATAGTCTCACTCTGTTGCCCAGGCTGGAGTGCAGTGGCGTGATTTTGGC
TCACTGCAACCTCCCCATCCTGGGTTCAAGCAATTCTCCTGCCTCAGCCTGCCAAGTAGCTGGG
ATTACAGGTGCCTGCCACCACGCCCGACTAATTTTTGTATTTTCAGTAGTGACAGGGTTTCACC
ATCTTGGCCAGGCTGGTCTTGAACTCCTGACCTCGTGATCCACCCGCCTTGGCCTCTCAGAGTG
CTAGGATTACAGGCTTGAGCCACTGCACCCGGCCAGATTTTAATCTAATTTTATTAGAACAATT
CAGTCATATGTTTTTTCATGCTATGTATATGAGAGTTCCATTATTCAGATACTAAACAAATGTC
TACTGTACATTTACTGTTCTCACTGATGATGCATTAGATAACCATGCACAAAATAAGCCTGGCT
GTGGAAACGCTTATTTGTTGGGAGGGTGCTTGTTTGGATCGATGATGAGAATAATTGTCTGAGG
ATGCTGAGGGACTCATTCCAGATGTCAATCTGAGGTCCAGATGTGCGGCCCTCCAATAGGACAA
ATAAGACTCTCAGAGCCTGGCTCTATTTGGGGATCCCTCAGTGACAACATAGTACCCCTGTGAG
CGTGCCTTTTCTATCTCTTCGAAGAGGGCAGTGGCATCCTGTCTTATGAGTCAGTGTGCACTTT
AGTGTGCCTAGTGACCCAAGACTTGCTTTAATTGTAGATAGATACTTACATATAGGAAATATTT
CTTAAGTAACAAATGAAAAACTTTAGAAGATTGAATTAAGGGTCAAGCAACTGTGATATGTCTG
AAAATCTCATTAGTGTTGTGCTGAAAGAAGGAAATATGGCATGCCTCTATTAAATAATGACAGT
GGAACCAAGTTTATTGCTTTGTTATTTTTACTGTGGAGTATTTTCTAAGATTATTTTTGCTTTT
TTTTTCTTTCATGTTTTGCTGAGATAGAAGGCCTGGAATCTGATCCTCCACTTCAGAGAACAGG
GGTGAGTAGCTAAGCCATTATCTTTTGAAATTCATATGTCATGTGCTCTTTGCTAGGTCTTTAG
GTCGTTTTGTACATCTTTTCAGAAGCTTATTGGAGGACATTTTCATGATATGTCCTTTTCCTCA
TTGAGACCCTCACCATGTCACCTACACTATTGAATCCTTATCATTTCTCTTTTAATTTTAACTC
TCTTTTGCTTTTATGGAAAAATGTAGAATTTAAGAGAATTTTTGGCAATTTCATATTGGATCAA
AATGTATTGTAGTGAAATCCAGGTGTGCCAAAATATTAACAGATTTTCCCCATCTGTTTAATTA
TTGGGGTTTCAGAATAGAGACTCCATGGTTCATAATATCTTTGTGGTCATACTACATTATATTT
CTGCTTCTAATTTAATTATTAAATATTGACTTGAATTAGTCTTTTCCTCATTGTTGCAACAAGG
TAAGTTATATAGGAAATTTTCTTCTCTTGATGGCATGTCTGAGATAATCATAGATATAAGACAC
CTGGCTGGTTTCTAGAATGCATGTAATTTTTATTTCTTGATCTGTGTGTTGAGTACTTGCTGTG
ATTGCATCATGCAAATACATTGAGCTTTACAATTTTAGTGTATGCACTTTTCTACATGTATATT
ATTCTTCGATAGAAAGTAAAAAAAACTTATCGAACTAGTCAAAATATTGGTTAATACATAAAAA
AAGTCTCAAGTAGATTGTGTATTACATGGTGCTTGTTGATTGATGCCCTCATAATAGATCAAGT
GGGTTCTCTCTTTAGCACAGGGCTTTTTAGCAAATCATGTCATGAGTAGTTACTCAAGTATTTT
TATTTTAACACATTTATATTTTTTCTATGTATATTCTTAAATTCTCTTATACTTTTTTCTCTGT
TATAAAAACATGCTGAACAATCTCAAGTCTTAAGGATTGCAGTATTGTCCCCACATATTCATGT
ATTTTGGTACTCAATTCTTTATACTTTCTTTGACAGATCACTTGAACTGGCACATGTCTCTTGT
TTTGCAGAGAGGGAATTAATGTGATACCTTCATGCTTTTCTATTCTATGTGCTACATAATTGAA
TATACAAGCAAATATAGTTGTTAAGATTTAGTGTGATTATTTCTACACCACATGCAAAGAAGTT
TCTCATAGATCTTAATAGAGGCCCACATGCATTGTACAGTTTAGAATTTGGGGAAATATTGATG
AAGTTGGGTAAAGTATAAAGCCAAAAGTCAGAACAGTGAACTCCTTGCTTAAGGATTTCCTTGG
AGATTACTTAGTCAATACACAACTGATAAATTTAAGTGCTTTTCACCTTTTGAGTTCTCGACAT
ACTAAAGCTAAAATGTGTTTCAACTTTTAATCCTGCTTCCCTGATTTTCCCTTTTTTAGTCTGA
GATCAAAGAGTTTCAGCCATAAATTACTGCCAAGAGTAATCACTTCATTTTAAGAAAGCTTAAC
AATATAGAAGAATATAAAATTATTTATGACAGATGTATTTTTAACCTTTTCCCCATGCTTTCCA
GAGGAAATATGTTTAATCATCTGCCCTATATTAGGGAAAAACTTTCTATGCTAATACAAGTATC
TATCAATCCATTTATCTTTCTATATAAGATGTATTGATCATAACCAATTAACTTTACTGTAAAT
GAGCTTTAGATTTGACATTTTGGTAGTAATATATGTTGTACAATCTCCTGAGGTCCTATAGGTC
TTGAGGTCTCTATGTCAAAAACTATAGATGTGCCAGTGTCCTCAGTGAATGTGAAGGACACCAC
ATTTTCCTTAGCCATTTCTTGTTTTCAGAATAATGGTTATCAACATTTTGCTACTGCAAGATAC
CATACATTTATAATCGGAATATGCCAGTTTTTATGCACTCATGCCTCTGTTTCTGTAGAGCATT
CCCAGAATGAGTAATGCTTGAAAATTAGGTCCATGTGATTTCTTTAATGAGTTATAGTCAAATC
ATGAAATATTCAGGTTACCATTATTTCAGTAATGTATTAGAATGTCAAGGTAAAGTTATCTACA
TTGTATATATACACACAACATAGATATAATTTATACAACATCTATATTTATACAACAGATATAT
ATTTATGTATATATTTATACAACATAAAATATATTTTATTATTTAAACATAAAATATATTTTAT
TATTTAATATAGATTCTTAAGTGATAAATATGTTTAATATTATTAAAATAGGTTAAAATAGGTT
ATAGTTAGTACAGTGAAAATTGGCAGCCCTTTTACAAAACATATGCCATAACTATAGCATTTAT
CACTGACAGTCATACCAGGATAGTCTTTTATTTCCAATCACTTAAATATTCCTAATTGCAAAAG
AAATTTGAAGACTAAAATTCAGAAGTTTTGAAAGAGCCATTGCCTGGGTAAACTATACAGGTTT
CAGTTTTATTTATAATAATTATGAGGCCAGGCGCAGTGGCTCACACCTGTAATCCCAACACTTT
GGGAGGCCGAGGCGGGTGGATCACGAGGTCAGGAGATCGAGACCATCCTGGCTAACACGGTGAA
ACCCCATCTCTACTAAAAATACAAAAAATTAGCCGGGCGTGGTGGCGGGCGCCTGTAGTCCCAG
CTACTCGGGAGGCTGAGGCAGGAGAATGGCGTGAACCTGGTAGGCGGAGCTTGCAGTGAGCCGA
GATCGCGCCACTGCCCTCCAGCCTGGGAGACAGTGCGAGACTCCGTCTCAAAAAAAAAAATTAT
GTATATATTTATAAATTAATACTTAATAAATTAATAACTTGTGATAGGCAATGCAAAGATGACA
GTAAAAGGACAAAATTGATTAGATTGATAAAGTCCTGTTAACATGAAGAAATTGACCAGGATAC
CATCCACACTATAAAGTTAGAGAAATTTATCAGGACAATTCCCTAAAATACTCTTCTCAATTTT
AACATTGTAACAGGAATTTTTAAAATTTTGGTATTATGTGTGTTTCCTTCCAGATAATTTGAAC
AGATTCATATTTGGTATTTTTAAAAGCCATATCTTTGTCCTTAGTGCTGGCAATGTATTCTTGA
GAATGAACAAATAAGAGATACGTAAAAGCATAAGAGAAGGTATCAGGTTGAAGTAGTCAATCAG
TTATACAGAACACAAAGAATTTTATCTTGTATAATGITTATATAGCTTTATAGAAGTGTGCTGA
AAGGGCTATAAAACATGGACATTATTATCTCATTGAAAGGTCCAATACGTACTGAAATACATGC
TTTTATTTTGAACCAACCACCCTATAAACGTTGTATGGCTTATTTAGATGAGAGCCCAGGTTGT
GTGTGTCTGTGTGTGTGTGTGTGTGTGTGTGTGTGTGTGTGTGTGTGTGTGTACCTGACAGGGA
AGCAAGAACATCGAGTTGCCAATGCACTCTGTCTATGGTTAGAATCATGCTGAAAACATGGCTC
CCCCAGTTCTGGAATGAGCCCACAGATCAAGCATTCCCCAAAGACATAGCAGGCTCAAATCCCT
GTGTACACAATATTTTATGATTATCTTATGTCAGTACTTTCAAAGTATACAGTTTGTGTGAAGA
CAAATCCAATGTCATTTTTCTTGGCTAGCCTATATGTGTGGTAAATCCATTATTTACTTACTTG
CTTCCTGAAAATTACAATTAGATTAACAAACTGCAGCAAAGTGGGCATGATGAGATAGAGATTG
AAGTGTAAGCTTATGTTAATGATGCCCTTGGTTTGGATAAACACATCTAAGAGAAAAATGGAAA
AACACACATGGCAGGGAAGCCTTGATAGAGCCAAAATATAGGATTGTATGTAGTAATGCAATCC
ATAGATGAGCATTTGGCAGTAATATTATTTTTCAGATATGGATAAAAATTGCTTAGGAGAGTAA
AGAGAGACAAAGTTGAAAGCAGGTTTATAGTAGGTGTTGTTTTAGTGTTGATCCCTTTTTGCTC
CAATAATCAAAGTGATAAATATTGAAAATTGATTCATGCAGCATTACTTACTCCATTCTAATTT
TTATATATGTCAAAAGTGCCATCTCCCAAACTGTGCTATCCCCTTCAGGAGAAGAGACTCTGCT
GAAGTTTATAAGGTTGACATATTGCCAGCTTCAATAATGTAAAGATGAAGTGTATACTGAATTC
TTAATGCAAATAACAACTCTATTGGAAAGTAACCCAGTTATAGAAGTGCTAATTTGTCAGGAGC
TGCCTTACCAAGATCATGATGAGTACAGTTATCTCAGGATTCTGAAAGATTGTTTTCCGATTTC
AACTAGTCTAGCTGAATGTTCCTTGATAGAAAGAGAGGACTTTTAGAATTGGTTCAATATGATG
ACCTCCTGAATTATCTCACATAGCCCGTTTGTACATGCCTTTCTTTTCTCTCAGAAAATGGCAC
TATCATAATAGCTTTCTTACACAGACTTCACCTTAGGGTTTTACATTAAGGGAGGGGTCTGGTG
TTTCATTTATTTTGAAGTATTTGTTGTTGATTGTGTACAGTGCTTGAGTAAAAAATTGAATATA
GAAACATCTAGAATATTTTTTTAAAGGATCAGTGTTTATAAAGTGAATTATTAGTGTCAATAAT
GTTGGGAAAGTTTTAAGAGAATATAGGAAACTTGAACATTACACAACTACAATGGGACCAAATT
GTGGGGTCTCATTATAGTTAATATTTATGTATTTTTTTCCAATTGATTTGTGTGCTTTTTTTCT
GCATGTTTTTGGCAGATAGAATGGCTATAACAAGTAACAGCATGTCAGGTAATAAAAATAAGCA
GAGCCCTATTCCTTTAAAAATCTTCACTGATGGGAGGGCCATAAAATAAGTCTTAATACATTTA
AAGAATTAAATTCATGTAAACCATGTTAATTTAATTCCACAATGATATTGAATTAGAAATAAGA
GGAATATCTCTTGAACATCTCCTAAATGTTTGGAAATTTAAATTAGCATTTCTGACCTATTTAT
TGGTTAAAAAAGATACAAAGAAAGGAAAATTGAAAAGTCTTTTGAACTGAATAAAAATAAAAAT
ATAGAATCTAAAACTTTATGGGATACTGACAAAACAGGATATAGGGAATAATTTATAGCACTGA
AATGCCTATATTAGAAAAGAAAAAAGGTTTTAAATCAGTAAATTTGTATTTTACCTTAAGAAAC
TTAGAAAAGAACAAATTAACCCAGACTTAAGTAAAATAAAGGCACTAATAAAGATAAGAGCAGA
AATCAATGAAATATAAAACAACAAAACACAGAGAAAAATTGAGAAAATTTAAAAATAGCCTAGT
GAGAAGATATTGATAAACTTGTAACCAGACCAATTTAAGAAAAAAAGTCAAAACACAAATACCA
ATATTTGAAAATGTAGGAGGGCAAATCATTACAGATTCTATGAATACTAAAATGATAATAAGGA
AAAATTATTTAAAAGGGGCATGTCAGCCAGGCATGGTGGCTTACCCCTGTAATCCCAGCACTTT
GGCAGGCCGAGGTGGGAGGATTGCTAGAGCTCAGGCATTCGAGACCAGCCTGGGCAACATGTTG
AAACCTTGTCTACACAAAAAGTACAAAAATTAGCTGGGTGTGGTGTTGCACACTTGTAGTCCCA
GTCACTTGGGAGGCTGAGGCGAGAGGATCACTTGAGCCCAGGAGGTTGAGGCTGCAGTGAGCCA
TGTTTGTACCACTGCCCTCCAGCCTGGGTGACAAAGTAAGACCCTATGTAAAAAAAAAAAAATG
TATGCCAACATTTTTCAATAACTTAAATGAAATGGAAAAATTCCTTGAAAGACACGAACTACAA
AAACTCAGTGAACAAGTAAATAACCTGAATAGCCCTGTATCAAGTAAATTGAATTTGTAGTTAA
AAGCCTTCCAACAGAGAAAACTTCAGGTACCTATAGCTTCATATGAAATGAAAAAAAAAATACC
AATCCTCTACAAGATTCCAGAACATTTAAAAGAAGGGAATATTTCCCAACTTATTCCATTTGGA
CAGCAATACCCAGGTAAGAAAAAGAGACACAGAAATTTAAAAAGAAGAATATACATTATTCCTT
AGGAACATAAATGCAAAGAAATCTAATCAAAATTTTGGCAAATGAAATGTAGAAATACTTTATG
ACCAAGTGAGAGTTACCCAAAGAATTTAAGGTTGGTTTTATATGTAAAGATCAACCAATATAGG
AAAATCACTTCTGGAAAGTCAGAGTAAGAAACTCCAAAAATCTACTCCTCCATAAAACCAATAA
CAGCCTTGATAGAAATAGTTGAAATTAATTTTCCAAAACTTTGGAAATTAACCAAAGGCTTACA
AAATTCCAGAGAACATTAATTCAAGAAAAATGGCTGAATCAGTAAGAACAGCCAGCTTTGTGGC
ATTTTAATATGACCCCTTCCCATGCTTTTCTCCCTAGTGCTGAGATAGTCTTAAAAATTAGCAG
GATAGCAACCACTGGAGAAGAAAGGTTTGGAAATTTCCCAAAAAGTTCCATCCCCATAGAATTA
TCACTATTTGACCTCTAAAGCCCAATCTATAGGATTTATATTCATTTGGACTGACTCAGAGCTC
ACTCAGTAAGGAAATCTCAATTTCAAGGTATTGGTCAAAAAGAATCCATGGCAATTGTTGACTA
TCACAACTGCCTGAAGTCTTGGTAACAGTTGGGATAAACAAGAAGCTGATCAAAAACTGAAAAC
TAAAATCTTGGGAATGAGATATCTACAGGATGCTTCAAAAAGCTTTGATACATTCCTGTTTATC
TAGAAAGCTACATGCAGGCTGGGTGCAGTGGCTCACACCTGTAATCCCAGCACTTTGGGAGGCC
GAGGCGTGCGGATCATGAGGTCAGGAGTTTGAGGCCAGCCTGACCAACATGGTCTCTACTAAAA
ACACAAAAATTAGCCAGGCGTGGTGGCGTGCATCTGTAATCCTAGCTACTCAGGAGGCTGAGAC
AGGAGAATCGCTTGAACCCGGGAGGCGGAGGTTGCAGTGAGCCAAGATTGTGCCACTGCACTCC
AGCCTGGGCGACAGAGCAAGGCTCTGTCTCAAAAAAAAAAAAAAAAAAAGCCACATGCATGTAA
TTGTTTACCTCTGGCTTTCCTTTTATGCTCTGGGCAAGCTAAGGAAGAGTTGTGAACTACCTAA
GTGCTGAATGGGAACCATAACACACACACACACACACACACACACACACACAGCACCTTAGTAA
AGGGTGAGAGGCATGTTAGTTAGAAGCATTAAAGGAAATCTCTTTCTAGTCATTATCTGTGCAC
TAACCTAACTGAGCAGAGACTTCAGTATCCACATACTACAGGGCATATAAACTTTACAGAATTA
GTCCAGGAAAATCATATCTAAAAAAAAAAAAAAGCAGTAACAAAAATAAACTCTGGGAAAGGGG
AGAATATGATTTAAAGAGTTGCCACATTATACATAATATGTCTAGTGTTCAACAAAAAATTACG
AGACATGCAAAGAAATAGAAAAATATGGCACAAAGAGGATAAGATGAAGTCAGTGAAACTATCC
TCGAGGAAGACCAGATGTTGGTCTTACTAGACACAGACATTGAACCAGCTATTAAAAATACGTA
CACAGAACTAAGAAAAACATGTCAAAAGGGTTAAAGAAAGGTATAAAAATAGTGTCTTACCAAA
TATAGACTACCAATAAAGAGATAGAAATTATAAGAAAAGACAACATGAAAAAATATAAAGCAAA
AAAATTAGACAATTGAAACAAGAGGGCCTCTATTCGCAGATTTGAGCAGGCAGAAGAAAGAATC
AGTGAACTTGAAGATATGTCAACTGAGATTATCCAGTCTGAGCAACAAAGGTGGGAAAAAATGA
AGAAAACTGAGCAACAAAGAACTGTAGAACAGCATCTCTCATACCAATGGATACATAAACTGGA
GCCCTAGAAGGATGAAAAAAGGAGAAGGAAAGAAAACTTCCCAAATTTTAAGAAAAACATTAAT
TTATATACCCGAGATGACCAATAAAATCCAATTAAGATAATCTCAAAGAGACCAACACCTATAC
ACATCATAGTCAGCGTGTCAAAAGACAAACATAAGGAGAGAATTCTTGAAGATAGTAAGAAAAA
AATTATTCATAACATACACACCATCCTCAATAAGTCTGACAATTGACTTCTCACTGTAAACCAT
GCAGGCTAAAAAGGCAATGTACATAACCAAAGTGATGAAATAAAAACCTTCAACCAATCATTCT
AGATCCAACAAAACTATTGTTCAAAAAGAAGAAATGAAGACATTCCTAAACAAAATCTCAGAGA
AATGTTCTCTATAAGACTTGTCCTAATAGAAATGCTAAAGGAACTCCTTCGGTCTGAAATAGAA
AGGCACTGGAGAGTAAATCAAATCCACGAGAAGAAATAAAGAGAACCAGTATAAGTAACTACAT
GTGTAAGTTTAAAACAAAGTATAAATTTATTTTGTTTGTAACATTTGTCTTTTCCTATTTGATT
TAAAATATAATCTCAATTATAAACTTGTGTTGATGGTATTATATAAAGATGTAATTTTGGGTTA
TAGCACCAAAATGGCAGAATAGGAATTTTCTGTAGGTGTTTCCCACATAAGTATCAATTTTGAC
AACCATCCATGGGCAAGAGTACCTTGTGGGAGTTCAGGAGTTGACAGTAAAACTTCAGCACACC
AGAGGAGTAAAGAAATCTAAGAATAGATTCATTGGAAAGGGTATAAACAGTTTCACTTTACCTG
CATCACCAACCCCCAAAAGTGGCACAGCTCAGTAACAAGAGCCCATTATTTCTTCCACAGAGGA
AAAGGAGAGTATAATAAGTAAGTGTCCAGTTTCTCAAGACATACAGGCCCCTGCCCAAGAGATC
CACTTTATTTTCATCTCACCCAGAATATTGAGGTGATCAGCAAGGTGGAGTGGTTGGGAGAGGG
TAAAAGCAGGAAAGAGAGATGGGGACTCAAACAGCAGCCCATACTTGGAACTGCCATAGATCCT
ACCAGTTACTTCATGGACTCCATCAGGAACCCACCTATAAGCCACAGGGGATGCATCCCTCCCA
TCTTGCCAACAAAACCCCGAATGCTCCAAATGCCTCACCCACTCTTTGGCTGGCTCCCAAGTGC
GCTCCTGTGAAAAGCGAGTGAGTATCTCTGCAGATGGCTTGCAAGCACATGTTGACAGCTGGCT
CCACTCTGTAGAACTGGAAAAAAGCTCACATACATGAGAATTTCAGGACACTACCCTAAAAAAA
AAAATGAGACTTCAGCACCTGGCCTGGCTTTATGCAACCTAGAGAAGGTGATATGATTTGGCTC
TGTGTCCCCACGTAAATCTCATGTCAAATTGTAATCCCCACGTGTTGGACAAGGGACTTGGTGG
GAGGTGATTGAATCATGCGGGTGGACTTCCCTCTTGCTGTTCTTGTGATAGAGTTGTTATGAGA
TCCAGTTATTTGAAAGTATGTAGCATGTCCCCCTTCACTCTCTTGCACTCCTGCTCCACCTTGG
TAAGACTTGCTTGCTACCCCTTTGCCTTCTGCCATGATTGTAAGTTTCCTGAGGCCTCCCAGCT
ATGCTTCCTGTATGGCCTGCAGAACTGAACTGTGAGCCAATTAAACCTCTTTTCTTCATAAATT
ATCCAGTCTTAGGTAGTTCTTTATAGCAGTGTGAGAATGGACTAATGCAGAAGGCATACAACCT
TTAGAATTTGCCCCCTTGAGGGAACAAGATGTGTGAAGCAGGTTCATCCATAGAAAATGTCTGA
GAGAACCTCAAAATCCCTAACCTGACTAACTGATGAAAGTGTTTCTCTCCTAAGGCCAGTCAGT
AAAGACCAGAGGGGGTGACTGTTTCTTTAAATGCAAAGGCAGCAGCACAATAATTCAAGAAACA
TGAAAAATCAAGAAAACATGACACCACCAGAAGAACACAATCATTTTCCAATAACCAACTCCCC
AAAAATGGAGATTTACAAATTGGTTTATAATGAATTCAGAACAATTATGTTAAGGAAGCTCAGC
AAACTAAAAGGAACACCAATAGACTACTCTGTGAAGTCAGGCAAACAATTCATGAACAAAACTA
GAAATTCAAAAAAGAGAAAAATTATCTTAAAAGAAAACCCAGAAGTTATGGAGCTAAAGAATAC
AATGCATGAAATGAAGGAGCGTATCAACAGCAAAGTTGATCAAGCATAAGAAAAAAAAAATCTG
TGAAACTGAAGACTGGCTATTTGAAATTATTCATCAGAGGATTAAAAAAAAAAGAATGAAAAGA
AATAAAGAAAGCCTACAGGATGTATAAAACACCATCAAGAGAACTAATATAAGGATTATTGGAG
TCATAAAGGAGAAGAGAGAAAAGGGTAGAAAACTTATTTAAGAAATAATGGCTGAAAACTCTCC
AAATCTAGGAAAAGATATGAGCATCCAGGTATATGAAGCTCAAAGATCCCCGTACAGGATACAT
TCCAAAAAGACTTCACCAAAACACATGATAATCAAACTGTCAAAAGCAAAATCAAGACGATGAA
TAAACCACCAATCACTAAGAGGGAGACAGGATTCTTATGTTGTCATTATTATTTACATATAATT
TCAGTAAATGTTATTGGAAAATTTATAATGTTTTAAAAAAAGAAATTTGAAAGCACCGAAAGAA
AAGAGACTCATCACATACAGGGAACCCTTTTAAGGCATTCAAGAGATTTCTCAGTAGAAACCTT
ACAAAATAGGAGAGAGTGGGATGAACTATACAAGTGCTGCAAGGAAAAAAATGCCAACCAACGC
TTTACCTGGCAAATCTGTTCCTCAGAAATGAAGGAGAGAGAAGAACTTTCCTAGACAAACAAAA
GCTGAGGCAGTTCATCACCACTAGACCTGCCTTACAAGACATACTAAGGGGAGTTCTTCAAGCT
GAAATGATATGGCAATAGTTAGTAATATGAAATGATAAACCTCACTGGTAAAGGAAAGTACATA
GTCAAATTTAGAACACTTTGATACTATAATGATGGTGTATAAATAATTTTACTGTGCTATGAAG
GTTAAAAGACAAAAGTATTAAAAAAAACCCATAGCTGCAATAGCTTGTCAATGCATACTACAGT
ATAAAAAGATGTAAATTAGAACATTAAAAACATAGCATGCAAGGGTAGGGAAGTAAAAGTGTAG
TTTTCATATGTAATCAAATTTAATTTGTTATCAGCTTAAAATAAATTGTTATACCTATGTTTTA
TGTAAGTGTCATGGTAACTATAAAGGAAAAACCTCTAGTAGATACACAAAAGAAAAAGAGAAAG
GAATCAAAACATAACACTACAGAAAATTATCAAATTACAAAGGAAGACAGCAAGGGAGGAACAA
AGTAAAAAGAGCAAGAAAAAATTTAACATAATGAAAACAGTAAGTCCTTACGTGTCAATAATTA
CTTTAAATGTAAATGGATTAAATTATCCAAACAAAAAAACAGACTGGACAAATGGATTTTAGAA
ACAACAACAACAACAAACACCGCACACACACACACACACACACAAACCACCCAGCCCCAACTAT
GTGCTGCCTACAAGAGATTTACTTCCACTTTAAGGACACATACAGGCTGAAATTAAAAGAACAG
AAAAAGATATTGCATGCAGATAGAAACCAGAAGAGAGGAGAGGCATCTATACTTACAGCATACA
GAAAAGATTTTAAGTTAAAAACTATATCAAAAGGCTAAGAAGGTCAAAATGGTGAAGCAGTTAA
TTGTTCAAGAAGACATAAAAATTGTAAATATTTATACACCCAATATTGAAGCACCTAAATATAT
AAGGCAAATATTAATACATATAAAAGGAGAAATATACAGCAATACAGTAATAGTAGTGAACTTC
AGTGCCTCCCTTTCAAAAATGGATAATCCAGACATAAAATCAATAAGGAAACATTTAACTTAAA
CTTCACTTTAGACCAAATGGATCTAACAGACATTATACTGAACATTTCATCCAACAGTGGTAGA
ATTCACATTCTTCTCAAGCACACATGGAACATTCTCCAGGATAGATTATATGTTAGCTCACAAA
ATAATATTACAAAATTTAATAAAGCTGAAATATCAATTATTTTGCACCACAATAGTATAACACT
AGAAATCAATAACAAGATGGAAACTGGAAATTTACAAATATATAGCATTAACATATTCCTGAAC
AACCAATGGGTCAAAGAAAAAAATCAAAATAATTTTGTGACAGCAAAGTAGAAACACAACATAC
CAAAACATACAGGACACAGCAAAAGCAGTTCTATGAGGTAAGCTTATATTGATAAACACATTTA
AAAAAAGATTTTAAATAAACAACATTACACCTCAAGGAACTACAAGGAAGAAAAAAAAACAAGC
CCCATGTTATCAAAGGGAAGGAACTAACAAAGATCAGACAGAAATAAATGAAACATAGACTAGA
AAAACAATAGAGACTATTAATAAAACTTAGAGTTAGTTTTTTAAAAAATAAAATCAACAAACCT
TTAGCTAGACTAAAAAAAGAGAAGACTCAAATAAAATAAAAAATGAAAGAGGAGACATTACAAC
TGATACCACAGACATACAAATTAAGAGAAAACTATATGCCAACATATTAGTTAACTTGCAATGG
GTAAATCCCTAGAAACATACAACCTACAAAAACTGAATCATGAAGAAATGGAAGATCTGAACAG
ATCAATAATGAATAAGGGAATTGAATCAATATTCAAAAATCTCACAAAAAGAAAAGCTCAGGAT
CAGATGGCTTCACTGGTGAAGACTGCCAACCATTTAAAAAAATTAATACCACTCTTTATTAAGC
TCTTCCAAAAAAATTGAAGAGGAGAAAACACTTTCAAATTCATTATAAGAGGCCAGTTTTACCT
TGATATCAAAGATTTAAAAAGAACACTTTGAGAAAGGAAAATTACAGGCCAAAACCCTTGATAA
ATATAGATGCAAAAATGCTCAGCAAAATACTAGCAAACCTAATTCAGCAACACATTATAATGGC
ATACATCATGACCAAGTGAGATTCATGCCTCGGATGCAGGATAGTTCAATATAATCAAATCAAC
AAATGTTACACTACTTTAACAGAATGAAGGATAAAAATCATATGATCATCTCGATGGTTGAACT
AGTTTACAGTCCCACCAACAGTGTAAAAATGTTCCTATTTCTCCACATCCTCTGCAGCACCTGT
TGTTTCCTAACTTTTTACAGATCACCATTCTAACTGGTGTGAGATGGTATCTTATTGTGGTTTT
GATTTGCATTTCTCTGATGGCCAGTGATGGTGAGCATTTTTCAAGTGTCTGTTGGCTGCATAAA
TGTCTTCTTTTGAGACGTGTCTGTTCATATCCTTCACCTACTTTTTGATGGGGTTGTTTGTTTT
TTTCTTGTAAATTTGTTTGAGTTCTTTGTAGATTCTGGATATTAGCCCTTTGTCAGATGAGTAG
ATTGCAAAAATTTTCTCCCATTCTGTAGGTTGTCTGTTCACTCTGATGGTAGTTTCTTTTGCTG
TGCAGAAGCTCTTTAGTTTAATTAGACCCCATTTGTCAATTTTGTCTTTTGTTGCCATTGCTTT
TGGTGTTTTAGACATGAAGACAGTGTGGTGATTCCTCAAGGATCTAGAACTAGAAATACCATTT
GACCCAGCCATCCTGTTACTGGGTATATACCCAGAGGATTATAAATCACGCTGCTATAAGCCAT
AAAAAATGATGAGTTCATGTCCTTTGTAGGGACATGGATGAAGCTGGAAACCATCATTCTCAGC
AAACTATCACAAGGACAAAAAACCAAACACCGCATGTTCTCACTCATAGGTGGGAATTGAACAA
TGAGAACACATGGACCCAGGAAGGGGAACATCACACACTGGGGATGGTTGTGGGGTGGGGGGAG
GGGAGAGGGATAGCATTAGGAGATATACCTAATGCTAAATGACGAGTTAATGGGTGCAGCACAC
CAACACGGCACATGTGCACATATGTAACAAACCTGCACGTTGTGCACATGTACCCTAAAACTTA
AAGTATAATTAAAAAAAATAATGCTGCTATAAAGACACGTGCACACGTATGTTCATTGCGGCAC
TATTCACAATAGCAAAGACTTGGAACCAACCCAAATGTCCATCAATGATAGACTGGATTAAGAA
AATGTGGCACATATACACCATGGAATACTATGCAGCCATAAAAAAGATGAGTTCATGTCCTTTG
TAGGGACATGGATGAAGCTGGAAACCATCATTCTCAGCAAACTATCACAAGGACAAAAAACCAA
ACACTGCATGTTCTCACTCATAGGTGGGAATTGAACAATGAGAACACTTGGACACAGGAAGGGG
AACATCACACACTGGGGCCTGTCGTGGGGTCAGGGTAGGGGGAGGGATAGCATTAGGAGATATA
CCTAATGTAAATGACGAGTTAATGGGTGCAGCACACCAACACAGCACATGTGTGCACATGTACC
CTAGAACTTAAAGTATAATAAAAAATAAATCATATCATCATCTCAGTAGATTTAGAAAAGCATT
TAACAATATTCAACATCCTTTCAGAACTAAAAACTCTCAATAAATCAGGTATAGAAAGAATGTG
CCTCAACACTATAAAAGCCACATATGACAAACCTGGAGGTAATATACTCAATGGTGAAAAGTAA
AAAGCTTTGACTCTAAGATCAGAACCAAAACAAGGATGTCCATTCTCACCACTTATATTTAACA
TAGTAGTTGAAATTCTAGCTAGAGCAATTAGGCAAGAAAAAAGGCACCCAAGTTGGAAAGAATG
AAGTTAAATTGTCTCTGTAGATGACATGATCTTATATATAGAAAACACTAAAGACTCCACCAAA
ATGCTGTTTTAATTAGAGCTTAAAAAAATAATTCACTAAAGTTGCAGGATACAAAATCAGTATA
CAAAAATCAGTTGCATTTCTAAACACCAAAAACAAGTTATCCAAAAAATTAAGAAAACAATCCT
ATTTGTGATATCATCAAAAAATAAAATACTAAAGAATACCAAAGAAACTGAAAATAAATGGAAA
TAAATGGAAAGATAGCCCATGTTCATGGATTAGATGAATTAATACTGTTAAAATGTTCATACTA
CCCAAAGCAACCTACAGATTAAGTGCAATTCCTACTAAAATTCCAATGACATTTTTCACAGAAA
TAGAAAACACACTCCTAAAGTTTGCATGGAACCGCAAAAGACTCAAATAGATGAAGCAATTTTG
AGCAAGAACAGTAAAGCTGGAGACATCACACTACCTAACTTCAAAATTTTATTAATCAAAACAG
CATGACATAAAAACAGACAGAAAAGACCAATGGAACAGAATAGAGAGCCCAGAAATAAACTCAC
GTTTATAGAGTCAACTAATATTCAACGAAGGTGCCAAGAGTACACAATGGGGAAAGTATAGTCT
CTACAATAAACAGCACTGGGAAGACAATATCCAAATGCAAAAGAATGAAATTACACCCTTATCT
TATACCATACACACAAATCAAATCAAAATTGATAAAGACTTAAATATAAGACCTGAAACCATAC
AATCTTTAGGCAAAAACTCATTGACATTGGTCTTGGCAATGATTTTTTTGATATGACACCAGAA
GCACAGGCAACAAAAGCAAACCTAAACAAGTGGGACCCTAACAAACTAAAAAGTTTCTGCACAG
CAAAGGAAACAATCAATCATCAGAATTAAAAGGCAATTTATGAAATGGGAGAAAATGTTTGCAA
ACCACATATCTGATAAAGGGTTAATATCCAAAAATATATAAGGAATGCATAAAATTCAATAGAA
ATAAACAAACAAATAATCCAATTTTAAAATGGGTGAAGAACCTGAATAGACATTTTTTCAAAGA
AGACTTACAGATAGGCAACAGGCATATGAAAAAATGCTTGACATCACTAATAATCAGGGAAATG
CAAATCAAAGCTCCAGTGAAATACCACCTCCAACTATTAGGATGGCTATTATCAAAAACTCAAA
AGAAAACAAGCTGGGGGAATGTAGAGAAAAGGGAAAAGAAGATCCTTATACACTGTTGGTGTGA
ATTTAAACTGGAATAGCCCTTATGGAAAACAGCATGGAGGTTCCTCAAAAAATTAAAAATAGAA
CTACTATATGATCCAGCAATTTCACTATTGAGTATATATCCAAATGAATTAAAATCACTGTCTT
GAAGAGGTATTTGCACACTCATATTTATTTCAGCATTATTCACAATAGCCAAGACATGGAATCA
ACCTAAGTGTTCATCAGTAGATGATTAGATAAAGAGAACGTGGTATATAGACACAGTGGAATCT
ATTTAGTGTTCAAAAAGAAGGAAATCCAACTTTTAAAATCCTTTAAAAAGTTAAACTCATAAAA
ACAGAGAGGAGAATGGCGGTTTCCAGGAACTGGAGGGTGGGAGAATGGGGAGATGTTGGTCAAA
AGGTACAAACTTTCAGTTATAAAATGAATAAGTTCTAGAGATCTAGTGTACAACAGCATTACTA
TAGTTAATAATAATATTTTTTATACTTGAAATTTGCTAAGAGTAAATATCAATATTCTCAATAC
ACACAAAACAGAACTATCTGAAGGCACTGATATGTTAATTATCTTCATTATAGTAATCATTTCA
CAATGTATAATGAATATCAAAACAATAGTGTACATCTTAAATATATACAGTTTTGATTTGTTAA
TCATACATCAATGAAGCTAGAAAAAATGTTGTAATTTTTAAAACAATAGTAATATAAATTAGGG
GTGAAGGAATTGACCTATGTTGGAGAAAAGTTTTTGTAAACTATTTAAATTAATTGGTATCCAT
TCAAGCTAGATTATTTTTAATTGTTAATTTAATTGTAATACTAAGGCAACCACTAAAAAAGCCT
TTAAAAAAATATAGCACTTGAGGCTGGGCATGGTGGCCCTCAATTATATATATAATATATATAA
AATATATATTATATATATAGTAGATGAACAACAATGGGATTTAAATGGTACACTAGAAAATATC
TGTTTAACAAAAAGAAAGCAATAGTGGAGAAATATAAGAACAAAACCATGTAAGATTTATAGAA
AATGAATAGCAAATTGGTTGACCTAAACCCTATCTTATAATTATATTAAAGATAAATGAAATAA
ATACTACATCAAAAGGCAGAGATTATCAGAATAGAGAAGAAAAATCCAAACCATAATTCAACCT
TATGTTATCTGTATTTAGAATATTTAGAGTCAAGACACAAATAGATAGAGTTCCATTTGGGCGT
GAAAATATTTACCATGCAAAATGTAATTAAAATAGAGCTAGAGCAGCAATACTAATATCTGACA
AAATATACTTTAACAAAAATTGTTGCTAAAGACAAAAAAGAACATTTTATAATAAGACACAACA
ATTATAAACATATACACCAAACAATAGAGCCCAAAATATACAAAGCAAAAACTGCTAGAATTGA
AGAAAGATAGAAAAATCAATGATTACAGTTGGAGGTGTCAATACCAAACTTTCAGTAATACACA
GAACAACAAGTCAAAAGCAAAAAGGAAATAAAAAACTACACATTATCATACAACACCTTAATAC
GATATCCAACAATAGCAGAATACACATTATTCTGAAGTGCACATGGAATATTCTTCAGGATGAC
ACATATTAGGCTGTAAAACATATCTTAATAATTTAAAAGAATTGAAATAATACAGCACATATTC
TCTGACTGCAAAATATTAAACCAATTACAGAAGAAAATGTGGGAAATTCACAATGATATGGAGA
TTTAAAAATACTTCAAAGTAACCAATGAATCAAAGAACAAATCACAAGAGAAATTAGAAAATAT
TTTAGATGAATAAAACTGAAGAAACAACATACCAAAATTTTGTGGTTGTAGCTAAAGCAGTGCT
TCAAGGGAAATGTATAACAGTATATCCCTAAATTTTAAAAAAATCTCAATCCAATAACCTAATT
TTTCACCTTAAGAAATGAGAGTGAAAGAGCAGACTTAACCCAAAGTAAACAGAAGGAAGAAATA
ATAAAGATTAGCATGGAGATAAGTAGTGAAAATAAAAACAATAGAGAAAATTAATAAACTTGAA
AGTTGGTTCTTCTGATATATCAGTAAAATTGACGTATTATTAGTTTGACCGAGAAAGAACAAAA
GAGAGAAGATTCAAGTTACTAGACATAAATGAAAGTATAGATATCACCTTACAGAAATTAAAAG
AATTCTAACAGAATATTGTGAAAAAGTGAATGTCAAAAAATTAAATTATATGAGATACACAAAT
CCCTCTAAAGGCACAAACTACCACAGCTGGTGTAAAAACATGAATACACCATTTACAGTTAAAA
AGACTGAATAGATAAAAATTTTAAGAAGAAATTGAGTAAGTAATTTAATTTTCAAACTATAATC
CCAAGACCAGATGTTTTCATTGGTGAATTCTACCAAACTTTAAAAGGATTAATATCTATTTTTC
ATACACTCTTTCCAGAAAATAGAAAAGGAGGGAACACTCTATAACTCACTGTATGAGGTCCGTA
TTACCCTTATATCAAGACCAAACATCATAAGAAAAGAAGACTAAAGACTTATGGTTTCTGCTCT
GATATGTTTGGAAGTCATCACTACTATTGTCACAAGGAAAAATCTGAACAAACTAAAGTCAACG
ATTTCTTAAACTAACCATAGAATTGAGGTAACGGGCGAAAACTGGAGATGTAGGCAAATACAAA
AAAAATCACAGTTTATCAGGAGCAGAAACTGCTGAAACCAGCAACTCGTATGAACATGTTAAGT
GGTAATTGACAAATTTCTGGAGATTGAATGTGGACTGGATTGAGAGTTAGGAACTCCTAAGTGC
CCAGTTTTTGATGACCCCACACACTTTTGTAAACTAGACTTCCAAGAGCCCCAGCAAGTTTCTT
ACAGTGAAGACTGCAGAAAAATCCCCTGATGCTTCAGATAGGAGGAAGGGAAAAGCAACTATTT
TGAAATAAGCCCAGGGGACAAGTAGTTATTTCTAAACACTCTCAGAGCATTTTCTTTCACACGG
CAGGGGGCTCCCTGCAAGGGAAGCTACTTTGCCTGAGCCTTGTCTGATGTAGGAGAAAAGGAAT
TGGATGGCTCAAGCTCCATCTAGCCTTTCTGATTTATATAAGGGAAGCAAAAAATAGGTTAAGA
AACTCTTCTGAAAGTCACAACTCAGATTTATTTTATATTTATTTATTTATTTATATTTTTTGAG
ACGGAGTCTCGCTCTGTTGCCCAGGCTGGAGTGCAGTGGCGCGATCTCGGCTCACTGCAAGCTC
CACCTCCCGGGTTCACGCCATTCTTCTGCCTCAGCCTCCTGAGTAGCTGGGACTACAGGCGCCC
AGCTAATTTTTTGTATTTTTAGTAGAGACGGGGTTTCACCACGTTAGCCAGGATGGTCTCGATC
TCCTGACCTCGTGATCCACCTGCCTCTGCCTCCCAAAGTGCTGGGATTACAGGCGTGAGCCACC
GCACCTGGCCTCACAACTCAGATTTAACCATAAGATTATAGAACACTTCTCCTCCCAAAACAGA
GATATAGATCAATGGAACAGAACAGAGCCCTCAGAAATAACGCCGCATATCTACAACTATCTGA
TCTTTGACAAACCCGAGAAAAACAAGCAATGGGGAAAGGATTCCCTATTTAATAAATGGTGCTG
GGAAAACTGGCTAGCCATATGTAAAAAGCTGAAACTGGATCCCTTCCTTACACCTTACACAAAA
ATTAATTCAAGATGGATTAAAGACTTAAACGTTAGGCCTAAAACCATAAAAACCCTAGAAGAAA
ACCTAGGCATTACCATTCAGGACATAGGCATGGGCAAGGACTTCATGTCTAAAACACCAAAAGC
AATGGCAACAAAAGACAAAATTGACAAACGGGATCTCATTAAACTAAAGAGCTTCTGCACAGCA
AAAGAAACTACCATCAGAGTGAACAGGCAACCTACAAAATTTTCACAACCTACTCATCTGACAA
AGGGCTAATATCCAGAATCTACAATGAACTCAGACAAATTTACAAGAAAAAAACAAACAACCTC
ATCAAAAAGTGGGCAAAGGATATAAGCAGACACTTCTCAAAAGAAGACATTTATGCAGCCAACA
GACACATGAAAAAATGCTCATCATCACTGGCCGTCAGAGAAATGCAAATCAAAACCACAATGAG
ATACCATCTCACACCAGTTAGAATGGCGATCATTAAAAAGTCAGGAAACAACAGGTGCTGGAGA
GGATGTGGAGAAATAGGAACACTTTTACACTGTTGGTGGGACTGTAAACTAGTTCAACCATTGT
GGAAGTCAGTGTGGCGATTCCTCAGGGATCTAGAACTAGAAATACCATTTGACCCAGCCATCCC
ATTGCTATATATATATATATATACCCAAAGGACTATAAATCATGCTGCTATAAAGACATATGCA
CAGGTATGTTTATTGCAGCACTATTCACAATAGCAAAGACTTGGAACCAACCCAAATGTCCAAC
AATGATAGACTGGATTAAGAAAATGTGGCACATATACACCATGGAATACTATGCAGCCATAAAA
AAGATGAGTTCATGTCTTTTGTAGGGACATGGATGAAATTGGAAATCATCATTCTCAGTAAACT
ATCGCAAGGACAAAAAACCAAACACTGCATGTTCTCACTCATAGATGGGAATTGAACAATGAGA
ACACATGGACACAGGAAGGGGAACATCACACTCTGGGGACTGTTGTGGGGTCGGGGGAGGGGGG
AGGGATAGCATTAGGAGATATACCTAATGGTAAATGACGAGTTAATGGGTGTAGCACACCAGCA
TGGCACATGTATACATATGTAACAAACCTGCACATTGTGCACATGTACCCTAAAACTTAAAGTA
TAATAATAATAAAAAAAGGCTACCTAAAAAAAAAAAAAAGAACACTTCTCCTCCCAACACCATA
TCACCACATCAACCAGGACTCCAGTGTAATAGCAGTGAATTCTAACTGAAAGAGGTGAAAGACA
CTGATTGTATTTAAGAAAGATCTTCTAAGGAAATCCAAAAATAGTAGGGGAGATCAAAACAAAG
ATACTAGAGGAAATTGAATATGTGACACCTATAGCTACAAAAAAATTAAACATAACATAGCCCT
AACCATATAAACATAAAACCTCACACAAAGACCTATTATCTGAGATTCTGTTGCCTGATACATT
GCGTTTTATTTCAATAAAAAAATTAGAGGGTATGTTAAAAAGCAGGAAAAGTTAGTCTAAAGAG
ACAAATTGAGCCTCAGAAGTAGGCTCAGATATGGCAGAGATTTGGCAATTATACCTAGAGTTTA
ATATAAATGATTAATATAATAAGTGTTCTAACAGAAAAAGGCAACATGCAAGAACGGATGGGTA
ATGTGATCAGCAAGAAGGAAACTCTAAGAAAGAAGTCAAAAGGAAATGCTAGGAATAAAAACCT
ACAAGAAATAAAGAATGCCTGTGATGGGTTCCTCAGTAGACTGGACAAGGTCAAAGAATCAGTG
GATTTGAAAATATGTCAACAAAAACTGCCCCACTGAAATACAAAAGAAAAATAGAATTTTAAAA
ACGTAACACAATCTCCAAAACAGTGGGACAATTACAAAAGATGTAATGTGCCTAATGCAAATGA
CAGTAGGAGTATAAAGGGAGAAAGGAATAGAAAATCTGAAGTAATAATGGCAGAGAGTTTTCCA
AAATTAATGCTAAACCACAGATACAGCAAGCCCAGAGAACAACAAGGAGGAAATTTAGTAAAGC
GTCTGCAACCAAGTATGTCATATTCAGACTGACAAAACCAAAGGTGAAGAGAAAATATTGAAAG
AAGACAAAGAGGAAAATAAATATCAAGAAAATACATACGAAATACATCATACATACATAAGAAA
TACATCAGACCATACAAGCAAGAAGAGAATGGAGTGAAATGTTTAAAATGTTGAAAAAAAAACT
ATCAATTTGCAATTCTGTATCCAGTGAGATTATCTTTCAAAAGTGAAGAGGGAAATGGCAGAGA
AGTCATCTCCAAGACCTATGGTTTCCCTTCACAGAAACACTGAAAAATATGAACAAAAGTGGTC
AGAATTAACTTTCTAAGAATTCTATAAAATGGTAAAATGTTTACACCAGTAAAGCAAATGCTGA
ATTGAGAAGGCAACTTAAAAAGGTGAAGAAAACTTCGTATTATTTTTATGTGTCCTTGCCCCAC
GTCCTTCCCTACCTTAGTCTTGAAGATGGCAGCCCACATTTCTACTGTGGGGCTCTGGTTTCTG
TTTCCTGGTTCAAGAGGGAGAATAACAGACCTTACTTTTAGTCATTATTATTTCCTTCTTTCTG
ATTTCCTTGGGTTTATTTTGCTCTTCTTTCTACTTTATTGAAATGAGAACTAAGATTATGATTT
GAGACATTTTTCTAATGTAAGCATTTAGTGCTATAAATTTCCATCTCAACACTGCTTTAGTCAC
ATCCCACAAATTTTTATATGTTGTAATTTCACTTTCATTTAGTTCTATTTTTAAATTTTTTCTT
TTTATACTTCCTCTGACTCACAGATTACTTAGAATTGTGTTGTTCAGTTTTCAAGGATATTGTA
GATTTTCCTGTTTCTCTGTTGTCTAATAGTTCTGTTCCATTTTGTACAGATAGCTCACGCTGTA
TGATTTCAATTTTTTAAAAAATTGTGCTTTGTTTTATGGCCCAGATATGGTCTGTGCTGTGAAT
ATTCCATGTTATTATAAAGTATGCCTGTTATATTATTATATATATATAATATATATAATTATAA
AGCATGCCCGTTTTGTATTGTTAGCAGAGTATTCTAGAAATGTCAATGAGATCTTGTTGGTTGA
TGGTGCTTTTCAGTTCTATATCTTTGCTAATTTTTTTTTTTTTTGCTTAGTAGCTATATGAGAT
TCTGAGAGAGGAAATTGAAGTCTCCAACCATAATTGTGGATTTGTCTATTTCTCCTATCAGTTC
TATCAGTTTGTGCATCACATATTTGAGGCTCTGTTGTTTGGTGCATACACAAGTGGAATCATTG
TGCCCTCTTGGTGGCTTATTTTATGATTATATAGTGCCTATCTTTGTGGTATTTTTATTTGCTC
TTAAATCTACTTTGTGTTATATTCATATACCCATTCTTTTTTAAAAAAAATTGTTTGCGTGATA
CATCTTTTCCATTCTTTTAATCTCAGCCTATCTGTGCCATTGAATTTGAAGTGAGTTTTCATAT
AGAGAACATATTATTGAATCATCATTTTAAAAATTCCTTTTGCCAATCTTTTTTATACTGAGGT
AAAATTGACATAAAATTTATCATTTTAAAGTGTACAATACAGTGGCATTTGGTAATACACATGT
TATGCAACGTTAACTCTACCTGGCTCCTAAATGTTTTCATCATCCCCAAAAGGAAACTTCATAC
TCATTAAGCAGTTAATTCCCATTCCTTCTCTCGGCCACTGGCATCCGCAAACCTACTTTTCTGT
CTCTATGAATTTACCTATTATGGATATTTTGTATAAATTGAATTATACAATAAGTGACCTTTAT
GTTTGGCTTCGTTCGCTTCGCATACTATTTTTCGATATTCAACCATGTTGTAGTATGTATCAGT
TTTATTTGAATAACTCAATTCTTTTTGTTGTATAGCTAAAAGTTGATTCCTAGGTCATAATGAT
AATTCTATGTTTAGTTTATTGAATAGCTGCCAAAGTTTTTCCACAGTGGCTCTGTCATTTTAAA
ATCCCACTAGCAATGGATGAGAGTTCCAATATCTCCACATCCTTACCAATATTGTTATTTTATA
TTTTTATAATTATAATTTTCCTAGTGAATACGCAATGGTATCTCATTGTGTTTTTGGTTTGCCT
TTCCCTAATGACTAATGATGTTGAGCATCTTACAATGTACTTGTTAACTATTTGTGTTCTTTAG
AGAAATGTCTATTCAAGTGCCTTGTCCATTTTAAAAATCGAGTIGTCTTGTTGACTTATGAGTT
CTTTAATACAGTAAACGCTTATCAGATATGATTTATAAGTATTTTAACCCATTCTGAAGGTCAC
CTTTTCACTTTTGTGGTAGACCATTATGCACAAAGGTTTTAATTTTGATAAATCCAATTTATCC
GCTTTTGTTGTTGTTTTTGTTGTTCGTGCTTTTGCAAAACCTAGTGTCATGAGGTTTTCTCATT
ATCTTTGGAGAATTTTATAGTTTGGGTCTATACATGTAGATTATTGATCTAATTTCCATTAATT
TGTGTGTATGCTACGAGGTAGGGGTCCAAATTCAATTTTTGCATTGAATTGAAAATTCATATTT
TCAGTTTCAAATTTCAACTGCATATTCAGTTGTTGCAGCACAATTTGTTGAAGAGATCATTCTT
TACCACAGGGAATGATCTGGGACCCTTGTCAAAAATCAATTGATCATAGATGTATGGGATTATT
TCAGACTTTAGATCTTGTTCCATGAATATGCCTATTTTTATGCCAGCACTGCAGTATTTTCATT
ACTGTAGCTTTATCATAAATTTTGAAATCAGGAAGTATGTATCCTCCAAGTGTGATTTTCATTT
GCTAGAGTGTTTTGACTATTTGGGGTCTTTGCAATTCCATATGAATTTCAGAATTGGCTTTTCA
TTTAAAAAAATGGTAGTTGAGATTTTCATAGGAATTATATTGAATCTACAGATCACTTTGGGTA
GTATTGCCATCTTAACAATATTGTATTCCAATCCATAAACACGGATGTATTGCCATTCATATCT
TTTTTTCTTTTCTTTCGGCAACATTTAGTATATGACACTTGTAACTCCTTGGTTAAATTTATAC
CTAAGCATTTTATCCTTTCTGATGCTGTTTAAATGGAATTATTTTATTAATTTCTCTTTTAGAT
GGCTTGTTGCAGGTGTATAGAAATAGAACTGATTATTGTGCTTTTATTGAATTATCTGAAACTT
TGCTGCATTTATTAACTCTAGTAGGTTTTTTTTTTCTTTAAAGTTGTCTATATATCTTGCTCTG
TGAATAGATAATTTTACTTCTTGATCTCCAATATGGATGCCTTTCTTTCTTTTTCTTACCTAAT
TGCTGTAGCTAGAATTTTCAGTATAATGTATGATAGAAGTTGTTCACAACTATCCTTGTCTTCT
TTCTGATCCTAGGGGTATAGCTTTCAGTCTTTCACCATTAAGCACAATGTTAGCTGTGGGGTTT
TCTTAGATGCCATTTAATATATTAAGGAAGTGCTCTGCTATTTCTAATTGGTTGAGTATTTTTA
TTATAAAATGGTGTTAGATTTTATGACTTTATGTACTGCATAATTGAGATTATCATGTGGTCTT
TTCATTCGATTAATGTGGTATATTTTATGATTTTCATATGTTGATCCACCTTTATATTCTTGGG
GCAAATCCCACTGTGTACACGGGTTTTTTAGGGTCTCCTAATTTTCTTCATTCTTTTTCTTTTC
TCTCCCTGAGACTGAATAACGTTAACTGACCTATCTTCAAGTTTACTATTTTTTCTTCTGCTGT
TCAAATCTGCTTGAACCTATAGAGTGAATTTTTCATTTTACTTATTGAACTTTTCAGCTCCAAA
ATTTCTCTTTGGCTACTTTGTATAATATCTATCTCTCTATAGATATTCTCTATTTGGAAAGACA
TTTTTCTCCTGGTTTTCTTTACTTATTTGTATTTTTTAAAGTCTTTAAGCATATTTGAGACAGT
TGATTTAAGTATTTGTGTACTAAGTCCATTGCCTAAGCTTTTGCATAGAGATTTTATATTAATT
TCTTTTTTTTCTGTGAACAGGTCTTTTTTTTTTTTTTTTTTTTTTTTTTTTTTTTAGAAAACTG
GACATTTTGAGTAGTATAAACGTGGCTATTTTAGAAATAATTTTCCTCCCTCCTTAGGTTTTGT
TTCCACTTGTTGCATGTTGCTGTTGTTTGCTCATTAGTGACTTTTCTAAAGTATTTTGAAAAGT
CTGTGTTCTTGATCATGTGGGTCCATTGAATTCTGTATTCTGTTAATTTCATAGTCAGCTAGTG
TTCTGAAAGTTCCCTTAAGTGCATAGAGCCAATAAAAGAAAAAGAAAAGAAACACAGAAAAAGA
AAAGAGGAAAAAAGGAGGGAAAGAGATTTAAAAAAATAATGTCGGTTGGGCATGGTGGCTCATG
CCTGTAATCCCAGCACTTTTGGGAGGCCGAGGCAGGCAAATCACTTAAGGTTGACCATCCTGGC
CAACACGGAGAAACCTTGTCTCTACTGAAAATATAAAATTAGCCGGGCATGGTGGCACATGCCT
GTAATCCCAGCTACTCGGGAGGCTGAGAGGTAGGAGAATCACTTGAACTCGGGAGGTGGAGGTT
GCAGTGAACTGAGATCACACCACTGCACTCCAGCCTGGGCAACAAGAATGAAACTCCATCTCAA
AAAAAAATAATAATAATAATGCCTTTGCCGATTTGCTCTGTGTTTGGGCCCTCATTCAATGCCT
AGTCAGGCCATTTACAACTCTCTCTTAACTTTCACTACCTGCTTGTGTACTGACTGAAGGCCAC
CTATAGGTGAAAGCTTAACATCATCTCAGATCTTTTTGGACCATGAATCCTACCCTGGGTATGC
ACATGATCTTCTTAATTTCCCAGTAGATGCAAGAGTTTTAGTGTTTTAAAAGTCCTTATTCCCT
CATCTATCTTCTTTTCTGACCTTTTTCAGTCTGCTTATTGTTCATCTGAACTGACATCCTTTGC
CCCAAGCGGCTGTGGCAAAAACTTTTACCTTTAAATGCTTTCACCACAAGCCACTTGGGAAGCT
GCCCCAGACCTGGGACTGCTCTGACCCTGATGAAACAAAGACAAGACCTTGTACAGCCAGGCAG
CCATAAGACAAGTCCATACCCAAACCACAGTTCTTTCAGAATAAGGTCTATATTGGATTCTCTG
GCCCTAGTAACCAGCATGAGTCTGGGCTTGCCATCTTCATGGCCACTTGTCTTAGTTTGCTAGG
GCTGCCATAACAAAATATGAGAGACTGAGTGGCTTAAACAAGAGAAATTTATTTTTTCACAATT
CTGAAGACCAGAAGTCCATTACTCTGAAATCTCTCTCCTTGGCTTGCAGATACTGCCTTCTTGC
CGTGTCCTCACACAGCCTTTTCTCTGTGTACACATCCCTGGTGTTTTTTAAGCCTGTCCAAATT
TTCTGTTCTTCTGAGGACACCAATCAGATTGGATTAGCGACCACCCATATGATCATTTTACCTT
AAGTACCTCTTCAAAGGTATCAGAGTAGTATATTTGCTACAGTTGATGAACCTGCATACAACAT
CATCACTTGAGGTCTGCAGTTTACATTAGAGTTCATCGTTAGTGTTATATATTCTAGGGTTTGT
TTTGTTTTGAGACAGAACAGAGTCTTGCTGTGTTACCCAGGCTGGAGCGCAGTGGTACAATCTC
ATTGCTACCTCTGCTTCCCAGATTCAAGCAATTCTCATACCTCAGCCTCCTAAGTAGCTGGAAC
TACAGGCGCACACCACCATGCCCAGCTAATTTTCGTATTTTTAGTGGAGATGGGGTTTCACCAT
GTTGGCCAGGCAGGTCTTGAACTCCTTGCCTCAAGTGATCTGCCCGCCTCAGCCTCCCCCAGTG
TTGGGATAACAGGCATGAGTCACCATGCCTGGCCTTATTCTATGGGTTTTGAAATGTATAATGA
CATGTATCCATCGTTGTAGTATTAGATAGAATAGCTTCATTACCCTAAAAGTCTTCTTTGCACT
GGGTTGAGTTTTAAAAGCTCTTTGATTATTTTATGACAGTTCCTTATTAGATATATCTTTTGCA
AGTATTTTTATCAGTCTGTGGTTATCTTGTCTTTTACAGAGCAGTAATTTTTAATTTTAATAAA
ATCCAATTTGTCAATTACTTATCTCATATGACTCTGGTGTTACATCTAAAATGTTACCACCATA
CTCAAGGTCACCTAGGTTTTCTCTAGGAATTTTATGGTTTTGCATTTTACATTTGGTGTATGAC
CCATTTGAAGTTAATTTTTGTGAGGTTGTAAGGTCTGTGCCTAGATTCATTTTTTTTTTTTTTG
GCATGTTACTTCAGTATTCATAAGAAACATTGTTCTGTAATCTTCTTTCTTATAGTATCTTAGT
CTTGCTTTAGTTTTTGGGCAATGCTGGCCTCACTGAATAAATTCAAAGTGTTCCCTCCTCTTCA
ATTATTTGGAAAAGTTTGAGAAAGACTATTGTTAACTGTTTTCTAAATTTTTGGTGGAATTTAC
CAGTGAACCAACTGGTCCTAGGCTTTTCTCCAGTAGGTGGTTTTGATTATGCTTTCAATCTCTT
TACAAGTTACACATCTATACAGACTTTTATAATTCAGTCTTGGTAGGTTGTGCGTATTTAGGAA
TCTGACCACTTCATCTAAGTTATCCAATTAGTTGGCATGCAATTATTCGTAGTTCTCTGAATAA
TCATTTTTATTTCCACAAAATTGGTAATATCCCAGTTTCCATTTTTTATTTCATTGAATCTTCT
TTTTTCTTAGCTAATCTAGCTAAATGTTTGCCAATTTTGTTGATCTTTTGGAAGAACCAACTTT
TGATTTATTAATTTTCTCTACTCTTTTTCTGTTCTTTATATTATTTATTTCCACACTAATCTTT
ACTATTTTCTTCCTTCTGTTGGCCTTTAATTTTTTTTTTTTAATTTTTAAGGTGTAAATTTAGG
TTGAGAAATTTTTTAAATGAAAGCATTTAGAGCTATAAATTTTCCTTCTGGTGTTCCTTTCACT
ACCTGCCATAAATTTTGATATGTTGAGTTTTTGTTTGTCTTGGAGTATTTTCTAATTTGTCTTC
TAATTTCTTCCTTGACCTATTGGTTATTTAAATGTATTTAATTTTTGCATATTGTGGATTTCCC
AGTTTTCCTTCTGTTATTGATTTCTAGTTTTATTCCATTGTGATCACAGAAGATATTTTGTATA
ATCGCAGTCTATTAACATTTATTAAGTCTTGTGGCCTAACAGAGGATCTATGTTGGAGAATGTT
CCAAGTGCAATTGAGAATACTATTCTGGTGCTATTAGGTGAAGTATTCTCTATATGTCTGTTAA
GTCCAATTCATCTATAGTGTTGACGTTTCCTGTTCCTTACTGATTTTCTGACTTATTATTCTAT
CCATTATTAAAAGTGGAGTGGTAAAGTCTCTATTATTGTAGAACTCTCTGTTTTTCAAGTCTAT
CAATATCTGTTTCATATATTTTGGAGCTCTGTTTGCTGCATATGTGTTTACAATTGTTATATCT
TCTTGGCAAATTGACCAGTTTCATCAACATAAAATATAATTCTTATTGTCTTCTAACAGTTTAT
TTTTCTTTTTACATAAAGCCTATTTTATCTGATCTTAGATTCCCTCACACTCCACCCCAGCACG
CTTTTGGTTACATTTACATAATATATCTTTTCCATCCTTTCATTTTCAACCTGTTTGTGTCTTT
AGATCTAAAGTGAATGTCTTACAGACAGCATAAGCTATGTCATTAAAAAAATCCATTCTGCTTA
TCTCTGCCTTTTGACTGGGGAGTTTAATCCATTTGCATTTAAAGTAATCACTGATCATTAAATA
CTTTCAGTATTTTGTTGTTTTATGTATGTCTTATAACTCTTTTGCTCTTCATTTCCTTCAATAT
TGCCTTTGTGTTTAGCTTATCTTTTTGTGTCACACTTTGATCCCCTTCTCATTTCTTTTTTATA
TTTTCTTTGTGGTTACCAGGAGGACAATGTATCAACTTTTAAAGTTATTACAATTTTATTTTTT
TAAATCTCTCCCATTCGGGGATTTTAGGAAGGTTAAATAATAATGTAAATGAGATACCTAGAAC
AATATAAGCATTCAGGAATTATTAACTCAATTCCAATCCTTCCTCCACCTCCACCTCTTTCTCT
GTGAGATTATAGAAAAGATGACAAAAAGGATGTTTTCTGAGCCCTTTAATTGTTGAGAATGATC
TTTGAGAAAAAGAAAAAAAATGAAAGCACTAGGAATGTACAACAGCCTGGAAGTATAATTAAGT
GTAAATTAAATAGATAAAAGTTATAAGCAGAGGAAAGTATAGTAGAACTCAGTATTTAAAAGAG
AATCAATGTGAAAATTATATAAATTTATGTAAAATAAAACTACCAGACAAATCTGATATCCTTA
GGATTTTTCTTTCTTTCATGTGATTTCTAATTGCTACATATGACACTAAACCATTGATCTGAGC
TGTAAGAGAAACTGGAAATTGTTCTGTTATCTTTTGTAAGATTTCTAGAACATTTTGCCCTCAG
ACTTAAATGCCAACGTATTTCTCACTTATTGTTTACTGCTTTTGGATTTACATATGATTTGATT
CTTTCTTATCTCTTATCCTTACAATGTAATTCAAACTGATGCCAATTTAAGTTCAATTGCGTAC
AAAAACTCTACTCCTATGCAGCTCCGCCCCATCTAATTTACATTATTGATGTCGCAAATTCCAT
CTTTGTACATAGTTTACATATTAACATGGATTTATACATTTTTATGTATTTGGTTTTTAAATCC
TGTAGAAAATAAAAAGTCAACACACCAATATTAAAATAATACTGGTTTTTATATTTGTCCATGT
GCTTACCTTTATCAGTGTTCTTTACATCTTTATACGGGTTTGAGTTACTGTCTTGTGTCCTTTA
GTTCCAACCTAAAGAACTCCCTTTAGCATTTTTATAGGGCAGGTCTAGTGGTAATGAACTCTCT
GAGTTTTTATTTAGGGATGTCTTAATTTCTAGCTCCTGTTTGAAGTAAATTTTTCTGGATATAC
AATTCTCTGTTGATTGATTTTTGTTCATTTTCCCTTCAGCTCTTTTAAATACATTATCCCACTT
TCTTCTGCCTTCCAAGGTTTCTATTAACAAAATTCAGCTTATAATCTTATTAAAGATCTCATGT
ACATGAGTGGCTTCTCTCTTGCTGTTTTCAAGATTCTGTGACTTTGGTTTCTGATAGTTTAAAT
ATAATGTGTCTTATTGTGGGTCTCTTTGGATTTATCCTAGAGTTTCTTGGTCTTCTTACGTTGG
TATATCCATGTATTTCAAGACATTTGAGTAGTTTTCAGCCATTATTTCTTCAAACAATCTCTCC
TCTTTGGGGACTTCCATTTTACCTATATTGGTTCTTTTGATGGTGTGGCACCAGTCCCCTAGAC
TTTGTTCACTTTTTTCCAGTCTTTTATTTCTGCTTCTCAGACTCAACAGCTTCAAGTGTTCTGT
ATTCAAGTCTGCTGACTCTTTCTTCTTCCAGCTCAAATCTGCTGTTGGATCCCCCCTTGTAAAA
TTTTTAATTCCATTTTAGTGTTTTTCAAGTTAAGTATTTTTATTAGGTTCCTTTTTATAATTTC
TTTTTGTTGATATTCTCATTTTATTACACATAATTTGTCTGATTTCCATTAGTTTTTTTCTTTG
TTTTCCTTTAGCTCTTTGAAAATATTTAAGACATTTTAAAAGTCTTTATCCAAGTTCAATTTCT
ATGGTTCTGTAGAGATATTTTCTGCCAGTTTATGTTCTTCTTTTCCATGGGCCATGTTTTCCTG
TTTCTTTGTATACTTTCTAATTTTTGGTTGAAAACTGAGCATTTGAAAATAGAGCCAACTTTCC
CAGTTTCTGCAGAGAGTCTTTATGCCACAGTATTCGTTCACTGATTAACTGGGTATATCTAAGC
TTAGGGAGCAGCTGAGTCAAAAGTTTAAGGTCTTCTCAGGTCTTTTCTGAGTATACATGTTTCC
TATGCCTGTGTGAAATGTTCTCAATTTCCCTATATAAACAGCTACTTCTTCTTTTTTTTTTTTT
TTTGAGATGGAGTCTCGCTCTGTCACCCAGGCTGGAGTGCAGTCATCTCAGCTCACTGCATCCT
CCACCTCCCAGGTTCAAACAATTCTCCTATACAGGTGTGTGCCACCACGTCTGGCCAATTTTTG
TATTTTTAGTAGAGACAGGGTTTCGCCGTGTTGGCCAGGCTGGTCTCAATCTCCTGACCTCAGG
AGGATTACAGGCTTGAGCCACAGTGTCCAGCCTAAACAGCTACTTTTGAATGCTTTAATTTCCT
GAATAGTCTCAACCCAGTTTTTCCTTGAGGTCTTAGGTGGTCCATTGTATGTCTCCACCCATAG
TTGCTTGCCCCAGGCATCTGTGGGTCTGTGGTACCACTGCAGCTTTCACCACCTGTAGCTGCCA
CCTTTCCCTATCTGAGATCCAGGTTAGGTGAGAGAGATCATTCCTTCACGCAGTCCCATGACAG
GTTGGAACATTTCAAATAAGGTCTGTTCTGCTCCTCTGGTTGAAGGGAGAAAATTGGGAACCGG
TTTCCCACCTTCTACAAACCAAGATCTCATGTTGCCACGGGAGTGGCAGGGCAAGTGCAAGTGA
AAATGCCATACAATTTTCTACCATTTTGAACGCGGGTTTTTCTTCAATGGTCATTTGCTTGGTT
GCTGTAGGCCTTTCACTGTTTTCCAGAGCTCCCATAAGATTACTTTAGCCAGTTTTTTGTTCTT
TCCTGATGCTTCCCTGGCAGAGTAAGGGTTGGAACTTCCACCATTTTGCTGATTCATAACTCTG
TAGTCAGTTTTAAATATATTGATACTTGAGTTTGTTTTATGGCCCAGAATATGGTCTTGGTAAA
TGTTTCACATATACTCAGAAAGAATGTGTATTCTGATGTTGTTACATGGGCTGTTCTATAAATG
TTACTTAAGGTGGTTAATAATGTTGCTCAAGTCTTCTATATTCTTGCTGATTTTCTTTTATTTA
TTTATTTTATTTTTTTTATTATACTTTTAAGTTCTAGGGTACATGTGCACAACATGCAGGTTTG
TTATATATGTATACATGTGCCATGTTGGTGTGCTGCATCCATTAACTCATCATTTACATTAGGT
ATATCTCCTAATGCTGTCCCTCCCTGCTCCCCCCACCCCATGACAGGCCCCAGTGTGTGATGTT
CCCCTTTCCTGTGTCCAAGCGTTCTCATTGTTCAATTCCCACCTATGAGTGAGAACTTGCGGTG
TTTGTTTTTTTGTCCTTGTGATAGTTTGCTGAGAATGATGGTTTCCAGCTTCATCCATGTCCCT
ACAAAGGACGTGAACTCATCCTTTTTTATGACTGCATAGTATTCCATGGTGTATATGTGCTACA
TTTTCTTAATCCAGTCTATCATTGATGGACATTTGGGTTGGTTCCAAGTCTTTGCTATTGTGAA
CAGTGCTGCAATGAACATACGTGTGCATGTGTCTTTATAGCAGCATGATTTATAATCCTTTGAG
TATATACTCAGTAATGGGATGGGTGGGTCAAATGGTATTTCTAGTTCTAGATCTTGAGGAATCA
CCACACTGTCTTCCACAATGGTTGAACTAGTTTACAGTCCCACCAACAGTGTAAAAGTGTTCCT
ATTTCTTCACATCCTCTCCAGCACCTGTTGTTTCCTGACTTTTTAATGATCGCCATTCTAACTG
GTGTGAGATGGGATCTCATTGTGGTTTTGATCTGCATTTTTCTGATGGCCAGTGATGATGAGCA
TTTTTTCATGTGTCTTTGGCTGCATAAATGTCTTCTTTTGAGAAGTGTCTGTTCATATCCTTTG
CCCACTTTTTGATGGGGTTGTTTTGTTCTTGTATATTTGTTTGAGTTCTTTGTAGATTCTGGAT
ATTAGCCCTTTGTCAGATGAGGAGATTGCAAAAATTTTCTCCCATTCTGTAGGTTGGCTGTTCA
CTCTGATGGTAGTTTCTTTTGCTGTGCAGAAGCTCTTTAGTTTAATGAGACCCCATTTGTCAAT
TTTGGCTTTTGTTGCCATTGCTTTTGGTGTTTTAGACATGAAGTCCTCGCCCATGCCTATGTCC
TGAATGGTATTGCCTAGGTTTTCTTCTAGGGTTTTTTATGGTTTTAGGTCTAACATTTAAGTCT
TTAATCCATCTTGAATTAATTTTTGTATAAGGTGTAAGGAAGGGATCCAGTTTCAGCTTTTTAC
ATATGGCTAGCCAGTTTTCCCAGCACCATTTATTAAATAGGGAATCCTTTCCCCATTTCTTGTT
TTTGTCAGGTTTGTCAAAGATCAGATGGTTGTAGATGTGTGGTATTATTTCTGAGGGCTCTGTT
CTGTTCCATTGGTTTATATCTGTTTTGGTACCAGTACCATGCTGTTTTGGTTACTGTAGCCTCG
TAGTATAGTTTGAAGTCAGGTAGTATGATGCCTCCAGATTTGTCCTTTTGGCTTAGGATTGTCT
TGGCAATACAGGCTCTTTTTTGGTTCCATATGAATTTTAAAGTAGTTTTTTCCAATTCTGTGAA
GGAAGTCATTGGTAACTTAATGGGGATGGCATTGAATCTATAAATTACCTTGGGCAGTATGGCC
ATTTTCACGATACTGATTCTTCCTATCCATGAGCACGGAATGTTCTTCCATTTGTTTGTGTCCT
CTTCTATTTCGTTGAGCAGTGGTTTGTATTTCTGCTTGAAGAGGTCCTTCACGTCCCTTGTAAG
TTGGATTCCTAGGTATTTTGTTCTCTTTGACGCAACTGTGAATGGGAGTTCACTCATGATTTGG
CTCTCTGTTAGTCTGTTACTGGTGTATAAGAATGCTTGTGATTTTTGCACATTGATTTTGTATC
CTGAGACTTTGCTGAAGTTGCTTATCAGCTTAAGGAGATTTTGGGCTGAGACGATGGGGTTTTC
TAAATATACAATCATGTCATCTGCAAACAGGGACAATTTGACTTCCTCTTTTCCTAATTGAATA
CCCTTTATTTCTTTCTCCTGCCTGATTGCCCTGGCCAGAACTTCCAACACTATGTTGAATAGGA
GCGGTGAGAGAGGGCATTCCTGTCTTGTGCCAGTTTTCAAAGGGAATGCTTCCAGTTTTTGCCC
ATTCAGTATGACATTGGCTGTGGGTTTGTCATAAATAGCTCTTATTATTTTGAGATATGTCCCA
TCAATACCTAATTTATTGAGAGTTTTTAGCATGAAGGGCTGCTGAATTTTGTCGAAGGCCTTTT
CTGCATCTATTGAGATAAACATATGGTTTTTGTCTTTGGTTCTGTTTATATGATGGATTATGTT
TATTGATTTGTGTATGTTGAACCAGCCTTGCAACCCAGGGATGAAGCCCACTTGATCATGGTGG
ATAAGCTTTTTGATGTGCTGCTGGATTCAGTTTGCCAGTATTTTACTGAGGATTTTTGCATCAA
TGTTCATCAGGGAAATTGGTCTAAAATTCTCTTTTTTTGTTGTGTCTCTGCCAGGCTTTGGTAT
CAGGATGATGCTGGCCTCATAAAATGAGTTAGGGAGGATTCTCTCTTTTTCCTATTTACTGTAC
ATTTATTCCACCAGTGACTAAAACAGGTGTATCAATAAAATCTGTTCCCTCAGGTTTTGCTTCA
GGTATTTTGAGGGTCTGTTATCAGGTGCATAAACAAGATTGTTATGTCCTATTCTTAAATTAAT
CTCCTTATAATTATGAAGTTAATTTTTTTTTTCTTGAGATGCAGTTTTGCTCTGTCGCCCAGGC
TGGAGTGCAGTGGCACAATCTCGGCTCAGTGCAACCTCTGCCTCCTGGGTTCAAGCATTTCTTT
GCCTCAGCCTCCCGAGTAGCTGGGGTTACAGGTACCTGCCACCACGCCCGGCTAATTTTTTTGT
ATTTTTAGTAGAGATGGGGTTTCACCATCTTGGCCAAGCTGGTCTTGAACTCCTGAACTCTTGA
TCCACCCACCTTGGCCTCCCAAGGTGCTGGGATTACAGGTGTGAGCCACTGCGCCTGGACCTGG
CCCGAAGTAAACTTCTTTACCCTTGCTAATGATCTTTGCTCTGAAGCATGCTTTGCTGGTATTA
ATATAGTCATTCCTTCTTTCTTTGATTCATGTTTGCAGGGTATATCTGTTTCCATTCTTTTACT
TTTAACCTATTGTCTTTATATTTAAAGTGCATTTCTTGTAAGTATAATTGGTTTCTTAAAATCC
AATTATCTGCCTTTTAAATGTCATTTTTATATGATTTGCATAAATATGATTATTATTACAGCTA
AATTGAAATCTGTCATCTTGCTATTTGGTTTCTATTTATCCCATTTTTTTCCCCTCTTTTTTTG
CTTTCCTTGAGATTGAACATTGTATTAGTTTTCTAGGGTTGCTGTAAGAAAGTGACATAAAGTG
GATGGCTTAAAACAACAGAAATTTATTGTTTCAGTTTGGAGGCTAGTCATCTGAAACCAAGGTG
TCATCAGGGCCGTATTCTCTCTGAAACCTGTAGGGAAGAATTCTTTCCTGCCTGTTCTAGCTTC
TAGCATTTTCCAGCAATTCTTGGCATTCCTTTGCTTGTAGATGTATCCCTCCAATCTCTGCCTC
TATCATAACATAGCCATCTTCTCCCTTTATCTGTCTATTCTTCTCATCTTATAAGAACATTAAT
TACTGAATTGGGGCCCAGCATAGATTAGGCCTAATCTCATCTTGAATAGGTTACATCTGCCAAA
GATTCTTCTTCCAAATAAGATCACTTTTACAGCTTTTACAGGTACTGAGAGTTAGAACCTCAAT
ATATCCTTTGGTGGGGACCGACCTCTTACCCATAAAAAGTATTTTATATGATTCCATTTTATCT
CCTTTTTAGGTTATTAACTACAATTTTTTTTTCTTTTTTTTGAGATGGAGTTTTGCTCTTGTAG
CCCAGGCTGGAGCGCGATTTTGGCTCACTGAAACCTCTGCCTCCCGGGTTCAAGTGATTGTCCT
GCCTCAGCCTCCTGAGTAGCTGGGATTACAGGCATGTGCCACCGTGCCTGGCCAATTTTTGTAT
TTTTAGTAGAAACAGGGTTTCACCATGTTGGCTAGGGTGGGTCTCAAATTCCTGACCTTAGGTG
ATCCACCCACCTCGGCCTCCCAAAGTGCTGGGATTACAGGCGAGAGCCACCACGCCTGGCTTTA
TAATTTTTTTTTAATTCAGTGGATGTTTTAGGGTTTATAGTATACATCTTTATCACAGTCTAGC
TCCAAGTGATATATCCCTTTATGTATAGTACATGACCCTTACAGTAGTGCATTTCCATTTTTCC
TCTCTGGCATTTAGGCTATTGCACACACATACACTCAATTCATCCCTTTGTGTAGATCCGTATT
TCCAGCTGCTATCTTTTGCTTCTGTCTGAAATATATGTATGATTTCTTTTATGACTATTTTGAG
AAATTTGGTTATGTGCATTTGTCTATTTTTCTTATGTACTTTTAGCTCATCAATCTTAAGTCTG
TGAGTTTATAGTTTTTAAAAACAAATTTGAAATTATTTGGCTATTATTTCCTCAAATATTTTTT
TCTGCCGCCCCTGCTTTCCCTTTCCTTAGGGATCTCTGATTTCCACCTATATTACTCTGATTGA
AGTTGTTCCACGTCTCTTTTGAAAATCTCTGAAAAATCTTTTATCATTTGGATAATCTGTATTT
GTACATCTTCAAGTACATTAATATTTTCTTTTGCAATGTTTAATCTGCTGTTAATCCCATGTAG
TGGATTCTTCATCTCAGGTATTGCTGTTTTAATCTCTAGAAATTCCATTGAAGTCTTACTTGAT
GAAAAAGGCAGATAAAAACAAATGTTGGCAAAGATATGGAGAAATCAGAATCTGCACACACTGA
TGATGGGAATGTAAAATGGTCAAGGCAATTTGGAAAGCAGTCTGGCAGTTTCTCAAAAGGCTAA
ACGTAGTTCCCATATGACAGCAATCATTCATCTAGGTATATACTCCAGAGAAATAAAAACATAT
CCACACCAGAACTTGAACATTAATTTTCATAGCAACATTATTCCTAGGGGTTTAAAATTTTTTA
CATTATTTTTATTTAAAATAGAGACAGGGTCTCACTACGTTGCATAGGCTGGTCTGGAACTCCT
GGAATTAAGCATTCCTCCTGCCTTTGTCCTGTTTTCTCCACTGGAAAAGAATAAAACTTTGTAC
ACTTGGACTAACAACTCCCGATTCCCTCCTTTACCAACATGCCCCACAGCCTCTAGTAACTTAC
TCTCTACTTTCATGAATTCAACTTTTTTAAGATTCCACATATAAGTGAGATCATACAATATTTG
TCTTTCTGTGCCTGGCTTATTTCTCTTAGCATAATGTCCTCCAGATTCATACATGTTGTCTTAA
ATGACAGGATTTACCTTCCTTTAAAGGCTGACTAGTACTTCATTGTGTATATGTATCACATTTT
CTTTATCCACTTATCTGTTGATGGGCACTTAAATTGTTTCAATGTCTTGGCTACTGTAAGTAAT
GCTTCAATAAACATGGGAATGAAGATATCCCTTCAACATATTGATTICTGTTCTTTTGGATAAT
ACTCAGAAGTGAGATTACTGGATCATATGGTTGTTCTATTTTTTTCAGAAACCTCCATACTGTT
TTTCATAGCGGCTGTACTAATTTACATTCCCACTAACAATGCATGAGTTCACTTTTCTGGACAT
CCTCCCCAACACTTGTTATCTTTCATCTTTTTCATAAAAGCCATTATATAATAGGIGTGAAGTG
ACATCTCACTGTGGTTTTGATTTGCATTACTCTAATAATTAGTGTGAGCATTTTTTTTTTTTCA
TGTACCGAATGTCTTTTGAGAAAGGTCTCTTCATTCCTTTGCCCATTTTAAAATCAGGTGGTTT
TCTTGCTCTTGAGTTGTTTGAGTTCCTTATGTATTTTAGATATTTACCCATTTCCAGATATATC
ATTTATATTTTTTCCTATTCTTTGAGTTCCCTCTTCACTGTGTTGTTTCCATTGCTGTGCAGGT
CTTTTATTTTGATGCCACCCCATTTGTCTATTTTTGCCATGCTTTTGCAGTCATATCCAAAAAA
ATCATTCCCAAGACCAATGCTGTGGAGATTTCCCCCTATGTTTTCTTCAGTAGGTGTACAGTTT
TAGGTCTTATATGTTAAGTTTTAAATCTATTTTTTTATATGGTGTAAATAAGGGTCTAATTTAA
TTCTTTTGCATGTGGATATCCAGTTTTCCCAACACCATTTATTGAAGACCCTGTCCTTTTATAC
TTTTCAGTATGCAGATCTTTTACCTCCTTAAATTTACACCTCAGTATTTAATATTTGTTGCTAT
TATGAGATTTTCATAATTTCCTTTTCAGATAGCTCATTAATAGTAGATGGAAACACTACTGATT
TCTGTAAGATGATTTTGTATTACGGAACTTTACTGAGTTTGTGTATCAGTTCTACCAGGTTTTA
GTTTTGTTCTGGTGGAGACATTACAGTTTTTTGTATATGGTTATGTCATCAGTAATTACAGATG
ATTTAACCTATTCCTTTCCTATTAGGATGCCTTTTTTTTCTTTCTCTTGTCCAACTGCTCTGGT
TAGGACTTCTAGTACTATGTCAAAAAGTGATGAGGGTCGTACATGGCCTCCATACCTAATCTGT
TGAGAGTTTTTACCATGAAACCAGGTTGAATTTTGTCAAATGCTTTTTCTGCATCCATTGAGAT
GATCATATGATTTGATTTACACCCTCCATTTTGTTATGTGGTATATCACACTTTTTGATGTGCA
TATGTTGAACCACCCTTGCATCCTAAGGATAAATCCCACTTCATCATGGTGAATCATTCTTTGT
ATTCGTGAATCCAGTTTGCTAATATATTGTTGAGGATTTTTGCATCCATGTTCATCAGGGATAT
TACTTTGTAAGTTTCTGTCCTTAAAGTGTCTTTCTCTGGCTTTAATAACAGTGTAACACTACCC
TTGTAAAATGAATTTAGAAGTATTCCCTCTGCTTCATTGTTTTGGAAAAGTTTGAGAATTTTTA
TTAGTTCTTTAAATGTCTGGTAAAATTCAGTAGTGAAGCTGCCTAATCCTGGGCTTTCCTTTGG
TGGGATACTTTTTATTACTGGCTCAATCTCTTTTCTTGTTATTGGCTTATTCAGATTTGTTTCT
TCATGATTCACTCTTTGTAGGTTGTATATGTCTAGGAATTTATTCATTTCTTTAGGTCATCCAA
TTTGATGGTGCATAACTTCATAGTAGTTTCTTATAATCCTTTGTATTTTGGTGATATCAGTAGT
AAATGTCTCCTCTTTCATTTCTGATCTTATTTGAGTACTCTTTTTTTCTCCTAGTCTAGGTAAG
AATTTGTTGATTTTATCTTTCAAAAAAAAAAAAAACCAACTCTTAGCAACTCTTAGTTTTGTTC
ATTTTTTTCCAGTCTTTATTTCAACTGTGATCTTTGTTACTTACTTCTTTATGCTAACTTTCGG
GCTTAGTCTGTTCTTTTCCTAGTTCCTTTAGGTGAAAAGTGAGATTGTGATCCTTCTTCTTTAT
TGGCGTAGGTTTGTATCGCTATAAATTTCCATTAGGACTGATTTTGCTGCATCACATAAGTTTT
GTTTCCATTTTCATTTGTCTCAAGGTAATTTTTTATTTACTTTTTGACTTCTTCTGTGAACTAT
TAGTTGTTTGGGAGCATATTGTTTAATTTCCACATATTGCTGTATTTTCCACCAGAATTGATTC
TTGTTCTTGATTTCTAGTTTCACGCCATTGTAATCAGAAAAGGGATTTGATATGATTTCTGTCC
ACTTAAACTTAAGATTAGTTTTGTGGACTAACATATATCCTGGAGAATGTTCCATGGGCATTTG
AGAACAAAATGTATTTTGCTGCTTCTGGATGGAATGTTTCATATATGCCTGTTAAGTCCGTTTG
GTCTAAAGTGTAATTGAAATCCATTGTTTCTTTATTGATTTTCTGTCTAGGTGATCAATCTGCC
CATGGTGAAAAGTAGAGTATTGAGGTCCCGTATTATAGTATTGCAGCCTATCTCCCTCTTCACA
TCATTTAAAAATTGCTTTATGTATTTAGGTGGGTCAATGTTGGGTGCATATACTTTTACAATTG
TTATGTCTTCTTGGTGAATTAATCCCTTTATCATTATATAACAAACTTCTTTCTTTTTATAGTA
TTGACTTAAAGTCTATTTTGTCTGATAGAAGTATAGCTACCCCTGCTCTCAATTTCCATTTATA
TAGAATATCTTTTTCCATCCCTTCACTTTCAGTCTATGTGTATGTTTAGTAGAAAAGTGAATCT
CTTGCCGGGCGCAGTGGCTCACTCCTGTAATCCCAGCACTTTGGGAGGCCAAGGCGGCGGGATC
ACCAGAGGTTGGGAGTTAGAGACCAGCCTGACCAACATGGAGAAAACCTGTCTCTATTAAAAAT
ACAAAATTAGCCAGGCGTGGTTGTGGGCCCTTGTAATCCCAGCTACTCGAGAGGCTGAGGCAGG
AGAATTGCTTAAACCCGGGAGGTGGAGGTTGCGGTGAGCTGAGATCATGCCATTGCACTCCAGC
CTTGACAATAGCAAAACTCCGTCTCAAAAAAAGAAAAAGAAAAAGAAAAAAAAGTGAGTCTCTT
ATAGGCAGTATGTGGTTGTGACTTAAAAAAAAAAAAAAATCCATTCTGTCATTCTATGTCTTTT
TGTTGGAGAATCTAATCCGTTTACATTCAAGGTAACTATTGGTAAGAAACTGCTAGTGTCATTT
TGTAATTTGTTTTCTGATTGTTTTGTAGGTCCCTTGTTTCTTTTTTCCCTATTGCTACCTTCCT
TTGTGGTTTGGTGGTTTTCTGTGGTGGTGTGCTTTGAATCCTTTCTTTTATAGTATATGCGATT
ACCATTGTATTTGCTGTAAGGCTAACTTAAAACATTTTATCCTTAGGCTACTTTAAGCGATAAA
AACTTGCCTTTAGTTGCATACAAAAACTCTACGCTTTCACAACCCCCCCGCCTTTCGTGATTTT
GATGTCAAAGTTTACACTTTTTTAAATTTGTATCTCTTATTGTAGCTACAGTTACAATTACTTC
TTGTAGCTACAACTTATTGTAGCTACAGTTGTTTTTAATAGTTTCATCTTTTAAACCTCCTAAT
AGGAATAACATTGCTTTACCTGCCACCTTTACAATACTAGAGAATTCTGACTATGAATTACTTA
TACCATTTAGTTTTTTACTTTTATGTTTCTCATTAACTAGCAGTCTTTTATTTAAGCCTAAAGA
ACTCCCTTTTAGTAATTCCAGTAGAGCAGGCCTAGTAGTGACAAACTCCCTTAGGCATTGTTTA
TCTGGAAAAGTGTTTATTTCTCCCTTTGCCAAGTAAAGTATTCTTAATTAACAGCTTTGTTTCC
TTCAGCACTTTGAATATACCATCCCACTCTCTACTGGCCTGTAAGGCTTTTGCTGATAAAGCCA
CTGAATCTGTATTGGGGCTACCTTGAATGTGATGTTTCTTATCTTTGCTGCTTTCAGTATTCTT
TGTCTTTGATTTTTGATAACTTGGTTGTGATGTGTCTTGGTGAACTCTTTAGGTTGAATCTGAT
CGGTGACCTCAGCTTCCTGTTCCTGAATTTTGTCATCTTTTCTCAGATTTGGGAATTTTTCAGC
TATTACTTCCTTAAATATGCTTTCTAGGCCTTTTTCTTTCTTTTCTCCTTAAGGACCTCCTATT
ATGCAAAAGTTAGCTAGCTTGATGTGTCCTGTAATTCTTATAGGCATTCTTTTTTGTTTTTGTT
TCTCATTGGATAATTTCAAATGTCTTACCTTTGAGCTCATTGATTCTTCTGCTTGATCAAGTCT
GCTGTTGAAGCGTTCTACTTAGATTTTCAGTTCAGTTACTGTATTCTTTATCTTTAGAATTTCT
ATTGTTTTTGTTTATATTTGTTAAACTTCTCATTCTGTTCAGATGTTGTTTTCCAAATTTCATT
TTTCTATCCATATTTTCTTGTACTTTGTTGAACTTAAGAGGATTAGTCTAAATTATTTGTCATT
TCACAGGTCTCCATTTCTTCTGAGTCTGTTACTGGAGCTTTCATATCCAATGTACACCAGAAAC
TTTGCTAATTCCACAGTAAATGTTAGAAATGGGTTTTTATTGATAAAATCAGTGGCTTGCTAAT
TAGTATGTTAGAAGCTGGCTTGCATTGGTGAAGTTGAGGGTATTCGATATACGTATCCCGGCTA
AAATACTTCTAAAGAACCTCTCCAGCTTTGCAAGAATAGAGTATAGGAATCTGAGGAGGCCTTT
AACATGGCCCTCAGAAAGGACACACAGGGCTTGACCTCTAAACATATAAACATATACAGAAACG
AGGTCAGAACATGTTTTGACTGATGCCAGTAGTAACACAGCAGAGCCTATTGCTGGTCACTGTT
ACCAATACCTTGTGTAATAAACCTTTCATTTAAATTGAGAGGTTGTAAGCCCCGAAAAAGCAAA
GGGCTTACAGTACCTCTTGCCTGAACACTCCTGTGTGTGCTTTCTAAGAAATGCTTTTAAAATA
ATGCCTGTGGCACAAATTGATTTGACACTGGCTCTCTTTATAAGCTGCTATCAAGGTTGTGGGG
AATAGCTAACTCTTACCCACTTTCCCTGACAAATAGGAACCCGTAGTTACCTACCAAAAGTATG
TAAAAGCTATATCCGACCCAAAGGCCAGTTAGGTCAAAACACCTTGAAAGGAGCTCAGTTTAAG
GAAATCAGTGGTGTCACAGTGCCCCACTAGCGTGTAAAAGTTTTCATAGTTTGACTATCGGATG
TGCTTAGTTTCACAGCTGAGTCTTACCTTGTGATACTTTTAGAGCAAAAGTCAAATCAAAGATG
ATTTAAAAAAACATTTCAAAGATTCATAAAGAATAAATGCCTCCTCAGGAAGTCTTCTGGAGTT
CTGCCCACTTCCCTTAGGTGGCTCTTTACTGTGCCCATCTCATAGCCTGTCTCCTTCCACTTGG
TGTATTGCAGAGTAAAGCTCACTGTTTACAGGGGTGGTAGAAAGTGTGGTCCTTTCCCAGACTC
CTTTTTCCCTTCCTCTTCTCATTTTTCAGAAGATGTGTTTGATAATAAACGAAACAAAATGACT
AACACATTGAGCTGAGCTACATAAGCAGATGTCAGTTTGACGTGAAGAGTTTAAAAGATCTATG
CATTATCTGGGGACTCCTCCCCCAGACCTGAAGGATCAGGTGCTGCCTTCTATGCCACCTGTGC
AGACAGCAAAAGAGGAAAACCATACCCACGTTCAGTATGAACAAAGGGGACATTTGAACTCTGT
GTGGACCCCTCATTGGAAGGGTGTTTCTTCTCCTGCTGCATCCACAAAGAGCACTCCTTAGCCT
TGCCTTTTGTCAGTTCTCTCTCCAATAAGGCTTGAGCAGAGACAACCCAGTGCAGTTCAGAGAG
ACTGAAGTCTGGTGTTCCAGGTCTGAGTCCTAGCTCTAGCTCTCTGTGTAACTTTGGGATGTCC
CAAAGTAACTTTTCACAACTTGATAGGIGTTAACTTGAATTTTGGATACAGGTGACTCTTAGCC
CATCCCCTCTCTGTGCTTCAGATATGTCATCACTTGGGCCATATGACCTCTGGACACCTTTCCT
ACTTTCCACAATTTCAGAGCAGCAGAGCAGACTGGAGCTCCTGCTGCCTCTGAGCTTCAGTGAA
TTATCACTCGTTGGAGGGAAGCTTCAAGCATTTTGTTATCTTTCAAGAGCAAACACAGTGTCTG
TCAGCAAGAATATGTAGCAGATGCTAGTGAACAGCAGTGATTAGGGTTGAATGCTGGATTTAAA
TATGGAGCTTAGGCTGTGAAGGAAGCCTGAAGAACCTAGAGCCCCATGAAGCTGCCCTCTGTGA
TATGTGAGTGCAATACAGTGAAAGCAAAGAGAATAAAATGATGGCTAACATGATGTTCCAAACT
TTAAACAGGAGAAAAACACACAATTCCATTATGTATAAGAACCCACACAGAGATCAGGAGAATA
ACCTCATTGGAGAATGAATGACCTGTGTGGGGAATTTAGGGTAGAGTTGAGATTGAAAAAATGG
GCCGAAGTCAGGTGGCCAAGGGCCTTAATACCTTGTATACAAGATGTGTAGTCAAGGAAGACCA
TGCCTTACTTATGCATCAATTCCCTTGGGCCTACAAAGAGCTGCCTAGCCTGGGACTGTTGTAG
AGAAAAGCTACAGTGTTCCAATGACACAGGGACTCCTCCATGTATGTATGAGTGCCCAGCTGGC
TCTGTAATAAATCTTATTTTTATTTATTAACTTTTCTTGCGCATTGGCTTGATGCATCAGTTGG
AAGTCAGAGGCCAAACGAAGTGAACACTGAGCCAAAGGAAGTTCTCAGCCTTTAGGGAGGCAGA
ATTCACTTTAAACACAATAAACAAATGAACTCACATTATACAGGAGAGAGTCAGAAGATCCCAG
TGGCTGGTGTCATCGGGCCATATTTGCCCGAAGTGCCTATTCCTTATAGGAACCCACTCCCAGG
GTTGATGGGCTACATCCTTAGGAGGCTTTATGCCTATGTTCTCCTGACCACTGGCTCCTCCAGG
GCTGGCCTTTTTTAGTCTCTCTGTAGAGGTTCCTGTAGCTGGTTGGATATAGGCTTTCACAGAA
GGGTCAGTGCCTTGGGTCTGGTTACAGGACTGTTAATCTTGCTTTGTTAAGAGTAATGTTATTT
CCCCATTTCCAAATTCTCCAGGGAGATGGAATGTCAAAGATAGTATGACTGTAGCACCTAAATC
CTGGGTTCCAGAAGCAGAGAAGAGAATACACTGAAGCTGTAGAAAGGCCTATTAGTCCATGTCA
GAGATTAACTGGATGCGAGGACCATTTCTGGGATGGTGTATACAGAACTGGAGAACTGGATAGG
GAGTCAGAAACAAAGAGCTGAAGATGATGCCTTTGAAGTTTCCAACGTGGATAACTAGGTCAAC
AGAATATTACTCAAGAAAGTATTAACATAGGATTGAGATGCAGTCAGTAGTAATGGAATTGAAA
TTTCAAAGTATATACCTCATGGATCTCAGGGGGTGTTGAGCTGATCACCTGGGCTAACACTCCT
ATGACCCTGGGGAAAATCAAATGACCTGGTACTGTAGCCATGGTAGGGGTGTCATCACCTTAAT
CCAACTGGGACAGTGCTGTTTGATATTCATCTGGAACTTGGTGCAGACCCACATTTTGCTGGGT
TTCACCACAACCAAGGCTTTTTTGATTCTTTTCTCTTTTAACATCAGTACATCACTGCAAAGTT
AATCCTCATATAATAGGAGATGAAACTAATTGCTTATAAAAACAAGATTTTTACAACACTAAAA
TTGTTCAAGCATATGGGCATATTTATAGTTGCAGGCAGTGTTTCAGATGCAGACTGTTCTTGGC
TGCAGTGGTTGTTTACAGGCAGCATCTGTTCTGATTAAATATTTGATGATTATCCCCGAATGTT
TTAAAGCATAGTACTGGGCTCTGCTGACTGTACAACAAACTGGCATTTTTGACCTATAGGGCAC
TGGGCTAGGAGATACAGTTCTGAGGGAAGTGAAAGATAGTTAACAACTGCACAACTGACCCTTT
ATTAGTTGCAATAAAGCAATCCAACCACCCAACCCACTGTGATGGCTTTCCTACTATTTAAGGT
TGGTGGTGTCAAAGAGACACCCTCCTGTACAGTGTGCAGTGAATCAACATCATTTCCACAAAAC
CTCCTTCCTGCACAAAGGAATTATCATACTTTGTTACGAAGTAAAATTTTCCTGTATCAGTCAC
AGGAGTTCACCAGTTAAGATACTGTTAGTTGAAGACTTCTGGGGTGACTTAATGAAATAGCTCA
GCCATCTGGTTTAAAAACTGGATTCTTCTATCCCTCCACACAGCTGTCCATGCACCTGCATCAT
CTCAAGGCTGGTCTCCCTGGTGGTAGAAAGGCTGACAGTAAAAACTGGAGCCACATGATTCCTT
GCTTAGGCAGTGTTTCTTCATACTCTCACATGAGAGCAGGCATGTTCTTTCCCTAGGCTCACAG
TAAACACTCTGTCAAATCTCACAGGCCCAAAGTGCTTTTGGTTATCCCCATTCCAAGCCAATCT
GTGGCATGGAAGATAGCATTACCCTGACTGCCTTAGACTAATATACCTACTCCACTTCTGGGGC
TGGGAATAATTTTGAGGTCAACCATCCAAACTGCATGACAGCCATTCCATGGAAGAGGTATGGC
CTAAATCTTTGGGGCAACCTGAATTCATGAAAACTCTCTTAGATTTATGTAACTATTTTTAGAA
TTCACTTCTGTATCATTTAATTTTACTAATAAAAATACCACCTTTACCCTAAATGTCAGCCAAG
CGTAAGGCTCCGTTGGGACAGAAGGAACTATCAAAGCTTTGTGTTTTTATACATTAGCAGCATT
TGACAAAGAAATAACTCTGAAGGAAGGAGAATAACCAGGCAGAGTCTAGATGCATGGAAAAGAA
GTCTTTGAGAAGGCTTCGCAGGCTGAAGGAAAGGGCAGGACTACTTCAGGAATACAACGTTTAA
GTAAAAGAGGTTGGGGTCTGTTGATCTTGAGGAGAGATGAGGATGGACTGGAAAATAGGAGTGA
GATATAGTAGGAGAGGAAAGGATATGGATGATGTATATACTGTATGGGTAACCATCAGTCGACC
CTAAGAAGATAATAGTTGTTAAATGGTTAGCTATTTAATGAAAGAAACCTGAACAAGTAATAAT
CTTGAGTTGCAAAGTGGCTGGAGTCACACAACAGACTAAATTTCTGGTAGAACAAATCCAGCAG
CTTATGTGAAGGTTACCATGTCTGAAGCTGGATAGAAAAGGTCTAACTTCCAACCAAAGTCACA
GTTCTTGAGCTCGGTACACAGAGACAGACTGCTAACAGCTGATGTGTCCTCAGCGAGAGTGTCC
TCATTTATATCCTCGTTCTCTTCTGCCTCCTTTTTTTGTTTTAAACTTTTGGGAAGTCTCATCA
TTCAATACAGTTCTAATATATCACAATAACAGAGGCGGCCCAATTTCTACAAATTGACTAATTC
TATCCCTGAAAAGTTCATACAATAAAACTATACAAAGCATCATTTTCAACCATCCTATAGAAAA
ATTTCCCTATTAAATTTTAACTCTAAATCCTCTATCTCTGTTAATAACCTTACTAAAAACTTCC
CCATCACTGCCTGCCAGGGAGATCAAAAGAAACCAAATTTAAGAAACCTCCAACACCTGTACCT
GACTGAAAAGCAAACATACAGACCTTTCAGTCCTGCCCCTACTATTCAGCTCCTATCACAGAGA
CATGGTGGATGTCCCCTTGGGAAGCGGATAGCTCTTAGGTGGAAGCTAGGCCTGCAATAAGCAG
GCCAGGAAACATGTCTCCCAGGCCCCATACTCTTGGCAGAAGCACTTGGCCACCAGACAGCATG
TCCTGTCAACCTACAGAGTTCTTAAAAACAAACACTGGGACCCAGAATAGTACCCTGTGGTCAT
AGTGCCCACAGTTCACTAAGCACCCTCACAGGTCTTTGACAGAACACTGACTGCCAGGTCACCT
GGTGGGCAGAGAAATGGAAGATTCCCAGGCCCAACTAGCATCTCAGGGAAGAACCACAAGCAGA
GCAACTTTCAGAGCTGGTCGGCCAGCGTTGGCACCCAGGGAAGCAAGTGCTATTCCATATTTGG
AGAGAACATAAAACTCAAGGAAACAGAGAAGTCCTACTCAATACCGTTCTCAACTGAAAACAAG
AGAACTTGCAAAAAAGAAACCCAGTTTTCTGGAGTCCATGGAGAAATATGAAGCCAATGCTCGG
CTAACAGAGCAGAAAGCCTTTTATAAATAATGCCAGTCAAAGCTCCAAAGGTCAGAGCTGATGC
ATGCCGGATTGTTCTGACAATTTCTTAGGTTTCTAGGCAACAAGGAGCTGGTCAAACAGCTCAC
CTCCAAGGACATACTTTATAATACCACCCTGGTAGACGAGAGCGAGGCAGCAGTGAAGGCAATA
GTCAAGAACAGGAGAGGGAGGTAAAGAACAAGATGCTCTTCAGACAGTCTGGACAAAGACAGTC
CCTACCCAGCTCTCAGGAGTTGCCTCCTTAAGAAGGTGGAGGCCCAGGCAGCTCCCAGGGTGAC
CCTAAAGACAGACTCCAAGGAAAAGGGTGTGAGGGCAATGGTGAATATAAGGAGGTCCCCTTTC
AGTAGGCAGAGCAAGTCAGTTCAGGTCTTATTTTTAGAGTCTCTGAACAGTGAAGAGAAGCTTT
CTGTGGACAGCATTCCACCACCATGGGAGGGGAAAGGTGCCATGAGAGACTTCTCCAGTGGGGC
ATACAAGCATTGTGCAGTGATCCCCAAGATCCAGCCACTGGCAGGAAATCGAAAGGCAAGCTTC
TTAAATACATAGTATATGGAGACAGAAGTGTGGAGCACTCCCAAAATGAAAGGCCAAGACCCAG
GAACAACCTCCACAACCTGGAGTATATACAAATGAACCCAGCCCTGCTGACTCAGATCCACACC
ATCCTAAAGCAGGGGTTTCTCATGAATGAAAGGGCTAAGTATTTGGTGGAGAGAATGAGAAGTC
TCCCACTGCCCACACATTTTTTCTTCAGAGATTTCTTTTCCAAGAGCCCCTTGAATAAAGGAAG
GGAGGGAGCACTGAATGCCCCAGAAATCAGAATGCATGGTGCGGGAAGACGACAGGAAATAGTT
CTCAAAGAGATCAGAAAATAAATTGGAAGCAATTTCATTACCACAAAGAACAGACATTTTTTCT
AAGGCACCCCTCCCCTTTCCACCCAATTGTCATTCAGCAACTACTGAATACTTACCAATGAAAA
ACGTTACCCCTGACCTCAAGGCTGCTCCTGAGCTCTGGTAGAAAATGCTTTCCTTGTCTATAAA
ACATGGCAAGCAAGGCAGGATTTAACAGTAAGGGCACAGTAGCACTGCAAGCCTTCAAATGGAA
ACCTGGAGACAAGCAGTGAGAACAGGAAGCAAAAGCAGGGCAGGTGAAGCTAGGACCAGGGCAT
CTGGAACTTTCCACACAGGTTGGATCTCCATGCCAGACAACAGTTTTCAAGGAAAAATATCTAA
GAGGAACATGACTTTGGGAAACTTTTTGGCAGTACTGCTTACTGTATACTAGAGAGTAAAAGAA
TTTGGGGAACATTCACCAATTTGCTTCTTCAGGGGCTTGGGTAGGGAACGTGAACAGGAACCTG
GCTCTAATTTCTGAACTTTTTTATCAGTAAAAACAATCCAACAAACGAAAGCTAGTCAGTGAGA
GAACTGGGAGGGTCTGCCCTCCTTCCCTGAGTCAAGCCTTCTGGGGGGACCTCCTGACATTTAA
TTAAGCAAAGACAACGCCCACTGAAGGAAGCTGACCTGAAAGTGACACGCTACTGTGAAATGAG
CATGAAGTGGGAGCTTGTTACATATATGAAATGGCCAGCGATCCTGAGCAAAGCGCTTCAGAGC
TTGAGACCTAAGTCTTCTCATCTATATACTGAGGGCTGGACAAGATGATCTGTCAAGCCATTTT
TATCCCTAATCCACCAAAATCCAATGCTTTAGTTTATTGTCACAAAAGCAGGTATCGAATGGCT
ATCCTGCAGTGCCTCCAATCAACATTCAGACTTTTTCCCTGAGGCAATATAAGATAACAGTTAA
CATGTTTTTATCAATTAGGTGGTCATGAGATAAATATATATGGGAAGTGGTAGTTTTTCACTTA
AATGCATATAATAATGGTACAGCTCTCTTTGAATAGTATTTGTTTATTTCTTAAATATTTAAGT
TCCTAAAGACGGTAAGAATAACCCAAGGAAGTGAAATCAATGTCACAAAGCACATGGCTAAATA
ACTGCAGGTTTGCAGTGCCATGTGTGAGATCAGATGACAGAAGGGAGAACTACCTTTAGGCAGA
GGCTTCTCATGTCCCCTGGAGTGGCCATGTGCTGTTCTACATGACTACTTCCACTTCGGTTATG
TAGAAGCTATTTAAAGCACACAGATGITTGTGATGAGAAAAAAGCCACCCTTAATTGAATAATG
GAAATTATAAGCATGATTTGAGGGTGGGGGTGGAGGTGGGAGTAGAGATGGGTAGAAAGGAGTG
CAATGGAAACAAAGGAGCCTTCATAAAATTCAAGTCACTTCTTAGGATAACGTGATTGATTTAC
TCACCACCTTCTTAGGAACATAAAGCAAACAAGTGGGTTTTCCTTTTACTGCTTTTCTGAAATG
AGCTACACTCAAGAAAGCAGCACGGGGGTTGTGCTGTCCCTGCACAGTGGCAGGAGAGTATGAG
GAGCAGGTGAATGCCACAACAGCTCCATCCAAGATCATTTTTCACATGCAGGAACCATTCTTAT
ACTACCCTTTACTGGTAATTTCTGTAGAAATCTGGAAGTCTGGTTGACACCCTCCTGTACAGTG
TGCAGTGAATCAACATCATTTCCCTTGGACTTTGCAATCACGGTGGCATTCATACATTCATTCA
ACAAGTATGTATGTACAGGACAGTGGAGTAAAGAAAACAGATAGTTCTTACTCTCACGAGGCTT
AAAATTTCAGGAGGGAACTAGGCAGTCATGAAGTAAACATAAAAACACAGATTGTAATAGGCAC
TAGAGAATAATGAAGACTTTGGGGAACACAATTTAAATTGGAAAAATATCTCAGTTCCGTTAAC
ACATGTTGAAGGGGAATAGCATTGTTTTGCTGGATTTGGGGCCTGGATGCAAGCATGTTGGGTA
TGCATTGTAGGGTAATGCATTTCCTTCCATTTGGGCCCAAGTGTATATTTACCACCCAGTTGTG
ATGAGCTGGGATCCTCCTGCTCAATCTCAGCTTGAAGCACTTGGAGGTTATCTGCCTGCTGTGG
GTGATTATTTTGGAGCAAGGTACTTCATTTGCCTCAAGAAACAGATTTGATACCACTACTGTGC
CCTTTTGGAACAGAGAAGTAGGCAAGACCCCAGTGTGAGGCAGAGTGATGGGATCTTTAGGGAC
ATAATTGATGATGTAACTGATGATGATTTTGGAGTTTATACATTTCCAAAGTTTCAAAATATTT
TCACTTGGTTGATTTATCTTTATGGTGATGACACTACGTAGATATATGCCCTTCTTAAAAGTTA
CAGTAAGAGGCTGGGAGCGGTGGCTCACGCCTATAATCCCAGCACTTTGGAAGGCCGAGGTGGG
CAGATCATGAGGTCAGGAGATTGAGACCATTCTGGCTAACACGGTGAAACTCCGTCTCTACTAA
AAATACAAAAAAATTAGCCGGGCGTGGTGGCGGGCGCCTGTAGTCCCAGCTACTCAGGAAGCTG
AGGCAGGAGAATGGCGTGAACCCGGGAGGCGGAGCTTGCAGTGAGCTGAGATTCCGCCACTGCA
CTCCAGCCAGGGCAATGAGCGAGACTCCGTCTCAAAAAAAAAAAAAAAAAAAAAAAAAAAGTTA
CAGTGAGAGTTGACATTGAGAAAAGGGAGGCCCAGCCAGGGTTATGCAAAGACACAGTAGGGAG
GAGGATGGGAAGTTTCTGAGACACCCCAGTAACTGCATGGGTCCACAAAGCACATGTACAGTCT
GCTCTATGCACTGCAGGTACAGCCACGAATAAGACAAAGTCTCTGCCCTCATGGAGCTTGGTGT
CTGTCTACTGGAGCAGACAAAAACAAACCTGCTTTTTCTCATTTGCCTCTACTGTGGGTTCCGT
GTATAACTCTCAAATGCTAGCATTTCCATGCATTCCATCCTTGTCCTTCTCTCCTCTCTGCCTT
TTCCAGGAGAATTTTCCTAGCCACAGGTTCAGCTATGGTCTATGTGCTGGAGTCATACATTTTT
ATCTTCTGTGTACGTTTTTCTCTAGACCCATATTTTTAATTGCCTTAGGACAGCTCTCCCTGGA
TATCCCTCAGACACAACTAAAACATCATTCAATTGTACTATTAATAATTTTCCCTTCAAAATCC
ACTCCTCTTCTCTATAGACCTAACAGCAACACTGTCCGTCCAGCTCTCAAAACCTGAAATGTGG
GAGTTGTCTTTGACTCTTATCTTTCCCATGCATGAGCACTGAATCAATCAGAGTCCCTAGCATG
TCTTGGACTGTGGCCTCCCTTCTATCTTCAGATTATCTGCCTTTTTAGAATTATGGCAATAACT
TAACTGCAAAGACCACATGTGCACTTGTGCTCTTTCAGTTTGTAATAAATATAAAATGAAAAGT
GATTATGTCTTTGCTTAACGTCTGCAAAATAAAAGTCCAAAGTCCTTGGTATGGTCTATAAGGC
CCTCATTATCTGACCTGCCTGCCTTCCCAATCTCATCTTAATCCCTTACAGCCTGACACTCAGA
CATACTAGACTTTCCACCTAACTCACTCACATACACCTATCCTGTATGTCAAGATTCGGCTTGA
CCTTCATCACCTACCTATCTGCAGTATTTTTGGATCTGAATTCTCTGCCCACATTGTACTTTCA
CATACTTCTTTTACCCTTAGTGGTTTGTACTTATGCCTGGTCTAACTAGATTGTCTCCCTGTAA
CAGACTTCTTGATTCAACAAAGCAGCTCTGAATCAGCCTGAAACCTATGGCACACTGCAAAATG
GCAAACATTCAATAGGTATTTGCCCAGTAAATGTTAATGAAAGGAAAAAAATTCAAACTTCAGT
TGGAATAGGATTAGAGACAAGTTAAAAAAAAGTTTCCCATAGAAATCTTCCTCATCGTAAATAT
CATCATCCTAAATGTCCCGAGTCTTTCCATGGGTCTAATTTACCAAATCATGAAGACTCTCTTT
CTCACTGTGTATGTGTAGTGGGAGAGGCAGAGACAGAACGGTTTTTTTTTTTTTTTGCAAGTCT
GCTCTCTGGATTCGTTTTTCTGGGGATGAAATCTCAGGCTATGTAGCTTTTCTGGTCCCTTTTT
GGTAATCAACAAATCATCAGTTTCTCTAGGTATTAAAAAGCCTACACTTTTGAAACACCAAGAG
GCCAAACTCTCTCTTAATTGAAAACATAATTCTGCCTCTTACTGAGGTCTTTGGGTGGGAAGCT
TAAAGACAGCAGTGTTGGGGCAATTGGTTTTTCTTTTCCTCCCTCCACCTTCTTTCCCATTCAA
GAGCTGTTGCTGCCCTTTCTGGAGGGGAGGGAATTAGAAAAAAAGATCCTGCCTTACCCAGTCA
CTGTTAATTATTACTTTGAGCCAGGGTGGGGAATGACTTTCTTTCCTGGAATACACCCTGCTGG
AAACCACAGCTGAGATTCTCTAAGCTGGCAGCTTCCAACCTCTCCTCCCTCAACAGCTTCAACA
TTATATGGCACTCAGCTGCAGGGTGCCTGGCTCCAGGCCGGCAGTCCATTTGTGCAGGGTAGTT
TTCAAGATGGCTCATAGCCCATCTCTTTCAGTGACCAGCGTGGCCTATGGGAAACATACATCTT
GACTCCATTATTGGTAGGAGTACTCTTTAGTTAACTGCCACAGGCCAGTTCAGACACGTCTTAC
CCTGAGAGCCTTTTCAGAGTCAGGTACCATCCCTGTGTCCCCCAGCATCTGAAGATCACAGGTG
AAGGTCTCCAAGTAGTTCACTTAAAAAATACCTCAGTAGGTTTAAGAACAGGGAAAGCTCCCAA
TTCATTCTGTCAGGAGTATGACTCTCATCCCCAAACTGGACAGATATATAATATGAAAACTACA
GACCAATATTCCTTATGAATATAGATGCAAACATTCTAAACAAAATACTAGCAAACCAAATCCA
GCAGCATAGAACAAGGTTTATGTAGCATGGACAACTGAGATTATCCCAGGAATGCAAAGTTAAT
TCAATATACAAAAGTCCATTAATACAACATATTAATAAAGGACAAAACAACATGAAAATCTTGA
TACAGAAGAAGCATTTAACAAAACCCACAGCCCCTCCTTTTTTTTTTTTTGATTAAAAAACACT
CAGTAAACTAGGTTTAATAAAAGAATTTCCTCAACTTGAAGCAAACCCACAGCTAGCTAACATC
ATACTCAATGGTGAAAGACTGAATGCTTTCCCCTTAAAATCAGGAACAAGAAAAAGATGTCTGC
TCTTGTCACTTCTATCCAATATTGTACTGGAGGTGCTAGCCAGCACACTAAGGCAAGAAAATGA
AATAATAGGAATCTAGACTGGAAAGGAAGAAGTAAAAGTATTTCTGTTCACAGATGCATGATCT
CATATGCAAAAAATACTAAGTGACAAAAAAAGAATTTTAGAAAAGCTACAATATACAAAATCAA
TATACAAAAATTAATTTTATTTCTAACACCAGTGATGAATACAAAAATAAAAATTAGAAAATAA
TCATATAATTAAAGTGGCATCAGTAAAATACTTAGAAAAAAATTAAAGACGTCAAGACTTGCAC
ACTGAAATCTCTAAAACATCACTGAAATAAAGACCTAAGCAAATGCAAAGACATCCCACAGTCA
TGGAACAGAAAACCTAACACCATTAAAATAGCAGTTATTTCTCAAATTTATCTACCAATTCAAC
TAAATCCCTATCAAGATCCCAGCTGTTTTGCAGGAATTGACACAATGATCATATAAAAATTCAT
ATGCAATGCAAGGGACCCAAAACAGACAAAATGATTTTGGGGAAAAAAAAAAAAAAAATGGAGG
ACTTGCACATCCCAAATCTAAACTTACTACCAAGCTACAGCCATCAAGACAGTGCGGTGCTGGT
ATAACGACAGACATATAGGGCAATGGAGTAAGACTGAGAATCCAGAAAGTCTTATATTTATGGT
CAACTGTTCTTTGACAAGGGTACCAGGACCATTCATGGGGAAATAATAGTCTTTTCAACAACTG
GTGCTTGGGCAGATGGATATACAGATACCAATGCACTTATGCAAAAAATGGATGAAATAGGAAA
CTTCACTACATTCTACTGCATGCTCGGTCATTTTCAATCATTTAGGTGGCAACACTGACAAGAT
AACAGAAAGATGGAGGTAATAACATGTGAAAGGCAAAATGGTTTGTTTTTTTTTTTAAAAATGA
CAGTCTCTATCATGAATTTACTTACACTCCAGGCAAAGGTTATTAGAAGAAAAAAAGATGTAAG
AAAATTCCTTAACTGAAATGTGGAAAGAGTATCAAGAGGAGACCCTAAGACACTCTGTAAGAAT
CCCAGTGACTCCTCACTGTTCAACTAAGAAATGTACCCCATTATGCTGTGCTACCACGGAAGCA
TTGGAGGCACTTTGGGGGTTGATGAAGTCTTCATGGATGAGGTACTTTAAATATCTGGTCATGA
AGAGTATTTGAGAAATATGACAAGTGAAGATGCTGGGTAGGAATGCAGCGAGGAAAGTGTGTAA
CACAAGGCCAAATTGGAAAGGTCAGTGAGGGGGCAATCTGTGGAGGCACTGAATGCAGAGGATA
TTAAAATTAGCAAGATAGTGTTCTAGCACAATGAAAAGGATTGGAAGAGGGAGATGAGAGTCAG
GGAGTGAAATTAGGCAGCTGCTAAGAATGTCCGGGTGAGTCAGAGGATCAGGACCTGTAAGGGC
AGTATAAAGAAGGAAGGAATGATGGGAGAGGTTTAGGGGGAAAGAAGTGACAACCTGTGACAAT
TGAGGAATGAGAGATTTTGATGATTGGGGTGAAGGTTACATTTCTTAAAAAGAAACAAAGAATG
GTCGGTCGATAGAGATGCAAGATGAAGAATTTTGTTTTTAGGACTACTGACTATGAGGTAACAA
AGAAATCCCAGAGACAGAGGTCTGAGCAAAAGATATGGGATTGGGGAAGAATCTTTGGGAGTGA
CTGAGGTTGACTAGAGGGAACTGGGCAAGAACAGGTAAGGACTTTAAGCAGAATTGGAGGAGTA
TCCATCTAAAATCTGGGTAAACTGGGATGGAAAAACAAGTAGCCAGAGAAGCAACAGCCCAAGC
TAGGGTGTTGTCAAGGTTATAGATGTTACTGATTTTGGCAATGAGGAGATTATAAGGATCCTTG
AAGACTGTACTTTCGGTGAAACTACCATGCATTAGTAGCCTTTCACAGTGATATCTACCCCACA
CCTGCATCAAAATCTTGCAGTGCCTATTAATAAATGCCAACCCACTAACGGGGAGGGGAGAAAA
GACTTGGGACTCTGTACTTGTAAAACCCTCTACCCTCCCCAGGCAATTCTTTACACACTAACAC
TGTTGAGGTAAAAGGAGACAAGTGAGGGAGCAAATGGAAGGTGTGTTTTTGGATTATACAGTGG
CTCATGGAAGGGTGGGAGGGGTACAGATGGCCCTTAGACACTGGCGGAAAGTCAGAGAAAAAAA
ATTACAGTAAGCAGGTAAAGGGATCAAGACTACACAGGGACTAGTCTTGGGACGACATTTCTTC
CTCTGACAGTTTGTATGGAATTCTTGAGAAAAATTCCTCGAAGGGGCCTGAAATCTCAGAATGG
TCATGTTTTTAATGGGAATAGGGAGTGATGCTGCCCCATTACAACCATCTGTTCTAACAGAATG
TCTGTACCGAGGAGGGATGAGTAACATCGGCAAGTTCTGTTCGAAGCCTTTTTCAAGTTTCTTT
TTGATATGTATCTATCTATCTATCATCTCCCTATACAAGCAAGCATCCCCAAAAGTAGTTGTCT
CAGGAAACCAGGGTTAGGATGACCAGCTCATTTGCTGGAGCTGCCAAGGTCCAGAAAAGTTTGG
CTGCAGGCTGTTTTCATGTTTTATGTATGTTTGAAATGTTCTATAATAATAAAAGGTTAAAAAA
GTTTACATTTATTTGGAAAACCAGTTACTTTAGTTTATGGTTCCTTTTTTTCCCTCCAGAGCTT
CCTGGAGATTGAGGTCTAATTCAAAGAAAACCAAAATATATAATAGAGTACCTGGGCAAAAAAA
GTACTTTTATAACATAACATTTGGGGTAGAGGAAGTATCCACTGTAGTCAAAATGTCTATGTTT
TGCTCTTCCTTATTGTTCAGGGACATTCCATTAAATAGTAATGAAAAGGCAGCAAAAGTAAGAG
GAGTGACAACATGCCCGGCATAATTAAGCAAGCTAGAGCAGCTATTCTGTGCAACCGACGATTT
TTTTTCTCTAAAATTTTAAGGGTAGGTTCATTCTGACTCTGTTAAAAGTCTACTTGATGTGAAC
AACTCTATATCTGATAACCTATTTCAATTACCACTTTAAAACTTGTCATATGGATACGTTATTA
CAATTGTAGAACTTTAATAAATACCATAATAATAAAACTTGAGAACTGAAGAGCACACATTTCT
TCACGAATTTATTATATAAAACGCCCTCAGAGTATTTAATTTCTCCTCACTTTAATTACACATT
AAGAAGCACAGTGGATGAGAAGCCTTTAAGATGACTACAGTTGCACGAAGGTCCCTTTCATCAA
GGTAGCGTATGTACCCTAACAGTGTTCTAAAGGCTGGCCCAGAAAAACCCCATGTTACCTTATC
ACAATATGGAAAGCATTGTCTTCTTTTTCCACTAAATTAAATTATGGTGAAAAGTGCCACAGTT
TTATTTAGCATTATGGTACATAACAAACAGTTCTGTCTCAATTATGAAAAAAATTAATTAAAAT
AATCCTGAAAGACATCCTTTTTCTCCCCCCAATGATTTGAAAGCTGCATTTTTCCTGCCAATTT
CAAACAAACAAATCATCAGGTTGATCTACAGTAATCAGTTAAAACAATCAGTCAATCAATCAAT
CAATCACCAAGGCACAAGCTCAGCACATTAGCTATAGCTTGTAGCAAAAGGATATATCAATGTC
TCACCTTAGTTAAAAATACATAATCCTTTTATTTTATAATGCAATAAAAGAAATTAACAACATC
ACATACACAGAAGACTAGGAAAGGGGAAACTACTTACTTCTGGAAATCAGTAATGTAAACCTAC
TTGTACTTTTCCATAGTACATGAAAGTAACGTTTAACATGTTTTGAATTAATTAATTAAATTTA
ATCTGTGGGGCTATACAATGTAATTCTTAGGAGTAATAGTTTCATTCATTTCCAGGTCAGCTTA
CTGTATGATTAAGTAACACAAGGCACAGTAGCCATCTTTTTCATTATGTTGCAACACTGATCAC
GTGCCTCGATAAAATGGCTGATTCAACAAGATGATGGCAACACGAAGGGGAGACTTTGGATTGT
CTATTTAAAATCTAGGTAATAAGTAAGTAATTAATAAAAACTCTATCTTAAGTGCACTTTCACA
TGCTTTTTGTTTATAATAAACAAACAACAAACTTCCTAACTTTGTTGCAATAGGCTTGACTACC
ATTTCATTTGGCCAAATGCACTTTCCCCAGTAAACTTAAAACAACAACGAGAACAACAAGAACA
AAAATCCCTGTCCTTTCATATACTAAGAAAGAGGATTGGCTACTGAAACAGTTCATTGCAAGAC
ACATGAAGACGACATACTGTGGCATGAGTTGTTTTTGTTTTTAATTTGTTGTGCTGTTACTAAA
GTTCTGAGGGCTGCAGTTAAAACATTCCAATTTCTCCCTTCCTTCCATCTTTCTTTATTGATTG
ATTCTCAAGATTTTGCACAGAAAACTCTTTGGGGGCTAGAACAGCAGTAATTGCATCACACTGT
TTTCAAGACTTCAAGTTTCAAAAGCAAATCATTAAAAAAAATACAGTTCCTGATTTGAGTTAGA
TACAGGGACAAAAAAGTAGCACATACTTGAAGGTTACGTGGTCTACAAATGGTGGCAATATTTT
CCTTGGGAGAGTAGTTCTGTTGGTATATATTTTTTAAATACTCAAAAGGCTCAACCTCAAGCAG
TAATAAACACAAGCAAAAGTGATTTAACCCTTAAAATAAATATTCAGAAAAACCTCTCTGTACA
TACAAGTGAAAGAATATGTAACACTTTCACGCAAAAAAATAATTATAATAATAATAAAGGATTT
GTTCATATATGTAGCTGAAATCTGCTGTTCCAGCCCACATGTCCCCAATAAAGAAGGGAGGCAC
AGACATAGGTGACTACTGTGGTTGACTATCTTACAGCCTTTTTGTACTGGGACACTATCACCAC
CAAAAATTTATCCCTCGTTATATTTTTAAAATTTTTTAAATTTTTTCTTTTTTTTTCCTTCCTT
TTTTTTGTTTTATTTTGTTTTGTTTTGTTTTACAGCATGCCAAATCCTTTGGCATACGTGATGG
CCTTCAACAATCTCTCTTTAAGTTTTTCTTTGCTTGAGTATTCCGGAAGTAAAAGCACATTAAA
GCAAGTATGAGATGTAGGTAACCTAAATAGAGAAAAGGGGAAAAAAACAGGAAAACTGTAAGTC
ATGGGAAATACACTTAGAATTAAATGCTCCTATTTTTAGATTGTATATAGTTGAGACGGTCTGC
AATGCAAACTATACATTAATGCAAATCATAAACTTTTTGTTGTGTAACTACCAAGTTGCCTTTA
TCCTATAAATTACTCAAAGCTAGTGACGATGATAAGATACTGTATCCATTGAGTTTTTACTACA
TAACAGATACCATTTTAGGTACTGAATTCTTACAGTTCATTTAACTAAATCTTTCCACAACAAA
ACCACAGAGAGGACATCAGTAAATGCACTTTAGAGGTTAGGTCACTGAGAAGTCAAGTAACTTC
CTCTAAGGTGGAGAAAATACTCAAACCTGTTTTACAAGACTGCAAAGTGTGTGCTCTTAAATGC
TTATTAGAAACACTGCTGGCAATATGACTAAGAAAATGATTTGATAACAGGATTCTAGCACAAT
CAAATGATAATCTTCCGAGCCTCAATGTAACCATTCTAAATAGATGATCATGTTATATGGCTTT
CAATTAACAAGCTGGGAATCAAAAAAGTAAATGAATCACACTAATTTGATTCCAAACCAATGTG
AGCCCCATAATAATTTTTAACTAGGGCAATTTCTTAAAAGTTTCCTCACACAATGACAGCAAAG
TATTTTCTCAATTGTCTAATATGATTTGGGGATTTGTATATAAAATCACGAATGTGCTCAGAAA
CTATAAAGACAGTTCATATGTATGTGACGAGGAATGCAAGGTTTTCGGTAGGTATACAGTCACA
AGTTAATAATTACCTACCTTTCTGTGTCTGGGCCATTTTTGGCTATAATCATCTTTAATTTTCC
TAGTCCTCCCACAGGTGCTCTGTCTGTGCCCGTTGTAAACTGCAAGAAGAGTCTTTTCTGTTCA
TCTGTAAATGAATGAACGATTTCCCAGAACTCCCTAATGAGAAAAAATACAATACTGGTTTCAG
TTTGGCATTCATTATGACTGGTACTAACATAAGCTTATGATTTGCATTAAAACTATATTAAGAG
ACAACTTGGAAGTTAATTATCAGGATAGTATCACTTCTGGTGATTTAAAAATTTCCAAGCAAAT
TTATCCTGAACAGCTCTGAATACGTAAAAATTTAGATTAGATTACAATATAGTAAAATATTAGT
ACTACAATAGTAAAAAATTGAGAAAACCCAAGTGTGTAGTAACAGGAAGTGACTATTTAAACTA
TGGTATAATCACATTATGCAGTCAGTAATGAGCAAAAGACAAAATCCTATGAATTGAAAAAGAC
TGAAATGAATATTTGGAAAAATTAACTCCAGGTGCCTTTGGGTATATATTTTATTTATCCTTTC
TCCTTTATTCTCCTGGTCTACTCATCCTTTAACTCAACTTTGGGAGGAAAAAGTGATACAGATT
AAGGACAAAAGAAAAAAAGACCCCCTGCCCCAACCAACTGGCCCCAAATGCAAACAACTACATA
CCTAATAGCAGTAATACAAAAGTTCAATTTTTATATGAACTAGAATACTTTAAACAAGTAATAT
GTGCTGTGTATGGAGGAGGAGAATTATTCTGACATTTCTGCCACCTGCAGTTATTTTAAGAGAA
TGATTTATCCTGTGGCATTCCTAAAATCTATGTAATAAAAGCTATGTTTTAATGACACTTATGT
TAGTTTGAGTTCTAAAAAACGAAATACAAGCTCATAAAGTGCAATCTTGAAGTTTATTTGAATA
ATCAGGCATCTATAAAACATATATACACTAGTTCATAGCTAAATAAATTTTTTTTTTTTTGAGA
CAGAGTCTTGCTCTGTCGCTCAGGCTGGAGTGCAGTGGCGCGATCTCGGCTCATTGCAAGCTCC
GCCTCCTGGGTTCGCGCCATTGTCCTGCCTCAGCCTCCCAAGTGGCTGGGACTATAGGTGCCTG
CCACCACGCCTGGCTAATTTTCTGTACGTTTAAGTAGAGGCAGGATTTCACCATGTTAGCCAGG
ATGGGTCTCGATCTCCTGACCTCGTGATCCGCCCACCTTGGCCTCCCAAAGTGCTGGGATTGCA
GGCATGAGCCACCGCGCCTGGCCTATAGCTAAATAATGTTAAGATTAAAAAATTAAAAAAAAAA
TTAAAAGTATTTTTAGTTGCTTATATAATATAAAATGCATTTTAATAGTTATTTAGAAAGTTCT
TTCCAGAGCCAGGTGTGGTGGCTCATGCCTGTAATCCCAGCACTTTGGGAGGCTGAGGCCAGTG
GATCACCTGAGGTCAGGGGTTCAAGATCAGCCTGACCAACGTGGTGAAACCCTGTCTCTACTAA
AATACAAAAATTAGCCGGGCGTGGTGGCAGATGCCTGTAATCTCAGCTACTTGGGAGGCCAAGG
CAGAAGAATTGCTTGAACATGGGAGGCGGAGGCTGCAATGAGCTGAGATCACACCATTGCACTC
TAGCCTGGGAAACATTTTGAGCCATCTCAAAAAAAAAAAAAAATTTCATCTCAAAAAAAAAAAA
AAAAAAAATTCCAGAATGTTAGCAGAGAACATCACACATCCCAAACCAGTAATTACAATTTTCT
ACCTTTTGATATTCTAATATCCCAACATACTATTTTTATTACCAAAATGCTGGCATTTTTGGTG
CTGCAACACCATTTATCTGAAATAACTTAAAAGTTTTATTAGAATTCTTGGCTTTCTCCAATCT
CTTGCCACTTCCCTTCCCTGCCCCTTTACAAATCCACCAGTCAGCAAGAGAAAACAAAAAAGAA
ACACACAACAACAGAAAACTGGACATGAAACTTGCAGGAGCATCTCTCAGGTAAGGAGGAGAGG
AATCGGACCCCAGTTCATGATACTCCTTTTGGGGAGGCTGACAAGAGAAAACAAGTCTGCTGTA
GCACAGAACCCTCCAACATAGCAGAAAAGCTGCCCTGCAGTGGGTGGTAATAAAGCCTCCTCAC
ACTGCAGGAGGGCTCTGAAGCTTAACTGAACCAGCTACACCTAAGGAGGTAGACCAAAGAGCAC
CACCGAAGAACAGAGACAGACCCTGCAGGAAGTGGCAGATCAGCAGTGGTCACAATGACCAGTC
TGGCTATATTATTACGGAGCTGTTTCTTTGGACTTTTAAACGAAACCTTTAAAAATTTTATACA
ATGAAAACAGGGAACAAAATGCATCTCTATTCCTATAAGTGTTATGTGTGTTACATTAACATTT
TGAATTAAACAAGAATGCATATATTTAGAAAGCTGAAGAAAACACGAAGAAGCTTCCAGGTTTC
CACATAAAAGTGGTGGCTGTATCGATCACATCCTACTCACATCTCTAAAAATCTACCCGGATCA
AAAAGGGGAAAATAAAAAAAAAACCTATGACTTAGGTCTAGAGTAAAACTAGGAGACAGAACAA
TAAACTCCAAATACCAAAGAAGTGGGGAAATGAGCAGGGTCCAGCAGGAGCTACATCGAAGAGT
GGCTTGTGCGGAATCATACTGATTAGGGATCACCCAAGGCCAGACCCCACCCCCTCCACCACCT
GCCCCTCAACAGAGCACTACTGAGTGTAGGTTAGAGCACTGGCAACAGGGTGGTTAAAGGAAGG
ACTACAGAAAAGTACTTGGGTCCAGCAATGGCAGGCTCAGGAAGGCACCATGCATGAAAGGAAG
GGGGGTACCTTCAAAAGCAGAAGTGTCCCTTAAGCGGCTGTAAAGGGGTGGAAGCAGCTAATGA
AAAAGAGTCTTCATTAAGCTACATGGAGCTGAAAAATCAGAAAGTGGTGATTCTGCCCTTACTA
AAGCAACAGAAGAGGGATCCCTTCAACCAAGACCCCTAAAGCCACACAATTCCTCCCTGCTCCA
CCATGAGGTCTAGTAATAACAGGTCCAGAAAAAAGTAACATCTGTTATAGAAACATAAACAAGA
AAAACAGAATTCGGAAGTCTAAATAAAGTTATTATGGGAAGAGTCTGGCGAATGAGAAGCAAAA
CTTTCCGGTAGACAAAAGTAGACCAGAAAAATTCAGTCATAAACAATGGGAAACTAGTAACACC
ACATTCCAACACAAAAGGAGAATGCAGCAGCAGACAAGACAGACCACCAGTGATGAGAAATACA
AATTCAGAAAGAAATGAGCACATCTTAAATAAAAATAAGAATACTAAAAAATACTGAAGAAATA
TGCAAGGTAACTTTGGAAATAAAGGCTAATTTTATTATAAGGTGACCAAAGAAGAAGCAGTATG
ACTAAAATTTCTACTGAGGACAGACTCTAGAAAATTAAAAAACAAAGAAAATGAATAAAATAAA
CATAACTAAAGAAATGCATATCAAAGCATAATAACGAAAAAGATTAATAGCTAACTACATAAAC
ATAAAACCTTTCATTTGGCAAAAAAATCCTATAAGCAAGGTTAAAAGACAAATGACAAACTGGG
AAAAAATATTTGCGAATTTACCAGCGTTAAAGGACTAATCTCCTTAACATATAAAGTTTCTAAA
AGTGAAGAAAACGACCAGCTGAGCAGCAAAATGAGCAAAAGACTATATAAAACTATAAACAGCT
CTACAAGAAAAAATGTCCATTATCATTCATAATTGAGGTAAGCTCAAAAATTGTAACTATAGTA
AAATACCATTTCTCAATTGGCAAATAAGTAAACATCCACATTTAACAACACATTCTGTTTGGAA
AGTTTTAAGAAAAGAGATACTGTTGTAAACTGCTGGTGGGAATGCAAAATGTTATTTTTCTGTG
AAGGAGGATTTGGCAGTATCTAGCAAAATTACACATGCATCTACCATTTGATGCAACAATCCCA
CTTCTAACAATTTATCTTAATGATACTTTACATATGTACGGAATAATGCATGAACATTAAGAGG
ATTCACTGTGACATTAGTACTAACAGCAAAAGGTTAAAAATAACCTCGATGCCCATAAATATTG
ATAAGCTATGCTGCATCCACACAAAGGAGCAGTATTCAGTCATACAAAACAAAATGGAAAACAC
CTCTGCAATAATGTGGGATGATTCTCAGGATACACTAGGAAGATTATTTTCCTAGTTCTGTCCT
CTGATAAGGCCTACAAGCAGTAACGTTCAAAAGCAAAGGCCATACTTTGCATCTAAAATCTGAC
TTCTAAATGCCATTCTCCAACAATTTATTGGAAAAATAACTTATTCCAGGCCTGAGAATGAATG
TTCGAGATTAGTTAGAAATCTCAAAATCTAATAGGGTCATTTTAAAAGCACACGATAGCTAAGC
AATTTGAATATCATTTAGAGTAATGACTGTGAAACGTATTAAATACAAAAACATTCATTAGTTC
GTAATATTCAAAAAGGCAGCAAAAACCAACCAAAAAACAAAATTAAATTGTCACTATTAAAATT
ATTACATTAACTCCTTACTCTAAAAACCTTAAATCTATTTTATCATGCCTTTTCTGTAGAACTG
TTTTTCAAGGTAATCAAATGGCACCCAATGATGAGAAAAAAGAATGCCAGGTATATATGTAGGA
CAAGCAGATGGAAATTTTTTTTCCCCAAACAGCCATTTTGCAATCCCCAATGAGATAACAGATT
AAGGTAATGATACTGATAAATACGAAAATCAGGTGAAGGGCAGGTAGCAGGTTCTGGAAAGATG
ATGATGGCAACGACATAGTTATTATTATTATTATTTTTTGTTTTATTCCCTCCCAACCTCCCCT
ACAAAAACAGCAAACTGAATGAGAAAACCAAAGACCCAGAGACATTATCTACAACAAAACCATG
AACCATTGCATATAATTGGACAGAAACAAAGCTCTGACAACTACAAGACTGGTTAGTAAGGAAG
CAGATGCAAGGTAACTGACTGGGTTTCTGACAGCCCTGAGAACACTGCCAACCCACTGGAAAGC
ACAGGCCAATCTGAAAACAGGGCTTAAAGTTTTAAAAAGTTCTACAGGATCTAATTTGCAGATG
ATTACAAAGGGATCATGATGTAGTAAGCTCTGGGCCCTTAAAACACCCGAATACCAAACCCCCA
CCAGAAACAAACCTTGTACCGAGGAAAAACTTCTGGGAATAACTTCCGAATGGACCAGGTCAGT
AATAAAAACCAAAAAAAAGTTCAAATATATGTGTGGGATAGAGGAGTAAAGAAGGCAGTTTCAG
AAAGCACAAGGGCATATTTTTGACCATTTTACAAAAACACAGACTTCTCCTCGTCCCTAAAAAG
CGAAAAAAGTTATCCTGGCCCATCTCTCCCTCGTCCAAGTATGAGAAACTCATTTCACTCACAC
ACACACACACACACACACAAATAAATAAATAACAGAACAGAGTGAAACAGTGTAATTATTAGAG
AAAAAGTATGTGCATATACATGAGAATGGTGTCCCTACAGAAAATCAAAGTACATGAAACAATA
TGCAAATAAAACATGAAAACTGTAAACAAATTTCAAACTGAGCTAAAAAGAATTTAAAAAATAA
CAAATCATTAGAAATGGGAAATTTCTGAATGAGGGTAACTGAAAAAAAGGAAGACATGACACAA
CTAAGGAGTAATTAGAAATGCAAGGAAAAAAATCCCATCAAATACAAAGAAACTAAAACTAAAC
TAGAAGAAACATAAAGGTGAATAAAACAGAACAATAATACCAGAACCTGAAGGCAGAAGAAAAA
TCTTAAAATCAAAAAGAAACAAACCCATGCTTGAGACTCTCCTTGCTGGTCCAGAGCCACAGTG
CTGCTGTGTACTGGCAGGAGGAATTCTGCTATAATTGGTCCTGGCAGTGTTCACCTTTCCTGCA
AGTGTTCCACAGCCCAGGGACACAATGTGGTCAGGAGCACTGCCAGGAACACCAGCAAGGGGGA
GCCTGCCACAACAGGCACAAGGCTCAGGAAGCACTCTCCAGCCCACGAAAATCTAGTGGGGGTC
CTCTTCCCTCACCCAAACACACTCTGCGCAGCT
SEQ ID NO: 2
Human SNORD115 genomic sequence.
GGGTCATGAGGAGGTTTGATGACATAAAAATTATGCTCATAAGTATTATGCTGAGACCCATTCT
TGGGAGGAGATGTGGGGGTGTCCTAGGCTGTGCAAAACATAGCTATGGAAACAGAAGTTATTAA
GAGAAAAGAACAAGCAGTCAGTGAGAATTTAGATAAGAGACTGTAATAGAATAAAAGTATCAGA
TTAGTGAATGGTTGTCCTAGTAATTTGGAAGAAAAGGAATAAAAAATGGAGTCAGACTACTAAA
TGGCAAAAATAAAGACTTGAAGAACTTAATTTTATACCTGAGCTTTGCTTTGATTGAAAGTTGA
GAAATCTTGTGATTTGTCGGTATCATCATCTCAAATCAACTTATTTTTCATCTCAACATGAAGA
ATATCAGCTCTAGCCTTTTGGAATTTCAGCAACTCAGATGCTTGTTAGAACTTATCCAGGACTG
CTTGCAGGACCCAAAATACTAATAATCCATATGCCTTTTTTTCCAGCTGAAATTAGCTGTCTTA
AGAGTTGTTTTTCAGTGCTCTGAGGATATCCACAAATATATCCCAAGGCACTCTGAAGTGTGTT
TTCAATCTTTGGCCCTGCACACTGGGGATGCCCACTGGATGTTGCATGGAGTGTTGCCTCCAGT
TGACTTTGATAGACACAATAAACACCACGTGAGAGCCCCCTCATGAAGGAGGGCTGAGGACTTG
GAGTCCAATTCTCCACCTCCCCTGACAGATAGGCTATTCAATGCCTTGGGAATCCGACATGAAG
GACTGCTTATAGCAGTCCTTGAAAACTTTCTACAGAATGTGAACTGGGAGAAGCTAAATGCTGT
ACTCACAAGCTGGGTGACTTCTCCATTGATTTTTACCATAGCTCACTGAAACATTTTCTAAATT
AACAGGTATAAATTCACAGACAGATCAGAATAGATCCTTGGTCCTTTCCATTTTTCTGGCACTT
TTGTTGCCAGGAATTCAAATACAACTAAGAGAAAAAGTGGGTAGCTAGCAAGAACAGATAGTTT
CTCAAATATTAGTGGCAGTAACCCAACTTTGAGAAAATATGGAGAAAGAGAAGAAAAAACCCTT
AAGACTGCAGTGTTAGCTGCCCAGTTACAATTATTCACAAAGTGAGCAAATACTTATTTGGGCC
ACCAATAAATAAGAGGGCACAAGCTAACGGGAAATTAATACGTCACTCATGTAAAAAGTCAACA
CATTAGAAAAAGGAATCTTGGATGAGATTGAGAAAGATGCAAGGGATAAAACCTGACAAGCTTT
ACAACCTCCTTGAGGAGGTCTTCCCTACTCTGTAAAATCAGACAGAAAATCATTGGCAAATGGA
GCAAAGCATGGGCAAAACTGATATAATGAGGAAACAGAGCTGGGAAAGGATAAATTTATGTGTC
TGTTTCTGTTTCCTATCTTTTAATCCTTTTTTACCCTTTCTTCTGGATACTGGAGCAACGTATT
CTGCAATTTCTACAGGCCTTTTCTGTTTTATCCTAAACTAGGAAAATATCCAGATATAAGTATT
TCTGGACTATCTGCTACCTCTTCTTTCTCCTCTGAGGTTTTCATGCAAATAGGCGAGGTCCTAT
TTGCATAAAATAGGAAGAATATGCCTTGTCTCCTAATATGCCCAATAATCTTTTAAGAAAGGGC
CTGCTGTGTAAACCAAGGGCTATGATCTTCTGTACTCTGAAGAGAGTGCTTTTGGAGCTTCTGG
GATGCAAGGTATCTACATTGCTAAATATGTGCTTTCTGAAACAAAAGATGGCTGTGGGAGAGTA
GGAGTCTCACTCAATGCTGGAGAAGCACTCCCAAGTTACAGAAAATAAACCCATATCTATGAGT
CCTAGGAACAAATGATAAAGATTTAACAAAAGAGATCAATACCATTGTTGTAACTTACAACAGA
GAAAAACCTTGGCCATGTATAGAACATTTTCTCTATTTTTACCTCAAATATGATGTAATTAGAA
AAATTTTAAAAAACGTGGCATAGTTAAAAAAAAATGGCACATTCACTTCATAATGCTTCCATAT
TGCCTGTGGGAAAGTTAGGAAAACTTGACAGGGAAGGTCAAACTGTTTATAGATTTATGCATGA
TCTTAGAGAAACAAGTIGTGATTCCTCTGACACTTGTAATTCCCAGCCTTGCCACTGTTTTAAT
ATCTGTTCCTGAGTTATCTAAGTATTTTTCTGTCATGGATTTATGTTCAATGTTTTGTTCTCCA
TGTTTAGCTGGGGAGTCGAGGTTCCTGTTTGAGTTTACCTGTGGAGCGGGAGGCTTTCAATATT
TGTGGCAACGGATCTTCCTGGAATTCAGAGAGTCACCTACTCCATTCTCTGGGCATTTATAAGA
AAATATAAAGAATATTCCTCACATTAGGATTCCATACTAATTGTTATTGATAGCTGCTAATAAC
TTACATGCTAGTAAAGAAGACATTTTAGCTTTACTAAATTTCTTAGACTCCCAGGACGTAAAAC
CTCGCTAGCTAAGTTGCAATATTACCAACTGAAGTAAAATATTTGGGATCTGTGTTATTGGCAG
ATGGAAGGTGGTTGCATCTTGAAGTCAGCCTAATATACAAATGAAACAACATGCCAAAAAAAAA
CTTCACCGATTTTGAGGATTAATTGTAGATCTTTGCGTTTTATGAAAAGGCTAAGCCTCTCACT
GAGATGTTACATGAAACATCACTAGAACTCTTTATTAAAGTCAAAATAAGGCTGAAGAAGTCTT
TTATGCCCTTAAAATAGCTGCTATTTCTTCACTTGTATTACTTTTTTAACCTTGTTAAAAAAGG
AGTTCTACCTTTTCTGTCTTGAGATTAAGGGTTACTCCAAAATTAGGCCTTGATTATAGGCACA
TTGCTTACTTTCCCAGGTTCTTGTATTTTGTCTCTCTGGAAATGCGATGATATTTATAAGGTAC
AGTGGCAACCACTCCCCTGATTAAAAAGGCTCATTCAGTCCTTGACTCTGAGAAATTTAACTTA
CTTACAGGTACTTCATGTTGTTACTGCTATTTTACAAGTTCATAAGACACAACACTTGTCAGTA
TGCTGTCAAACGAGCTAAGAACAAGTCTTATTGTGTGATCCTAATGCTATTCTAGGAAGGTGTA
ACCTTTTGACAGCTATTCTGTGGCCAGATCTTGACAGAATAAGTTGAACTTGACTGTCTCAGAG
TGATTGAGGAAGACACTGGAGCATGGTCTAATTTTTGTAATATTCCTATTCCCAATGTTGACTG
AATATTCATGAATATATTCTGTGTGAAAGACCCACAAAGGAATATAAAAGCATGTTCTACGGTA
ATCAACCCTCATGAAGCTTAGAGGCTTCTGTTTTTCGAAACATAAAATCAGCCCAAGTGGCAGA
ATTGTAGTTATTATACCAGCTGTTAAATTAGCCACCAGTTATAAAGCAGATATCTATACTGGTA
ATAACTATATGITTGTGGTCTGTTATGTGACTAGCCAACTTTGGAAGAATAGATGCTAACGTCA
ATAGGAACTAAAATATTACATGAGAAGCTAATTTCTGATTTAGTAGAGTCATTCAAACTTCTCT
AACAGATTTATGTAATTTATTGTAGAGCTCACAGTGGAAAACAAGATTAACTCTTTAAAAAGAA
ATTAATTTGCAGACCTAGCTGTGAAGGGCTTCTGACGTTTAGACTGGGGAAATCAAACCTCTAT
CACTTCTGTCCAAAACCTCCTATTCATAATTTCAGAAATAGCCAACTCTCTTAAGAATTAGAAA
ATTAGGAATGAAGGAGAACTAAGATGAACACCATAGGAAAATGAGAATTACATAATGAAATCTT
TCCTTTATTCCGATTGTTATATGATTGAGTGGCTTTTTCATGCCACCAACAAGTTCGTTCAAGC
AAAAGGAATATGCTGACAACTTGAAATCTTTGTAATAGCATCATATAATTCAGGAAATAATATG
AAGATATGCTCTTTGTCAGAAAAATTCTTTCCTTACCTAAAATAAGTCAAGAAAGCCTACTAAG
GCCCATGATACCTAGGATCTGCTGGCACAAGAACTTTATAGAACTTCCACCTTTAGAAGATAGT
AAATATTTTTACCAGTGTTGTGTATTGGTGTCTGGATAGGCTAAAGCAGATGCTCAAACAGTGA
TCAAGCTCTTACTGAATCATATCATCTCAACCACTGGAATTCCTGAATATCTGGAATTCAGTAG
GGGAATCATTCTGTATTTGTGGGAATTCGGGAATTTTGTAATACTCTACAAATACCAATAATTT
ACCTACCTGTTATCCCCAGTCTTCTGAACAAGTGGAATGAGTGAACCACATACCCTAAAGAATT
ATTTAGCTGAATTATTTAGCTAAGTTATGTAAAACCTTACCATCCGTTTCATTTAAAATAAGAT
TAAATCCCTCTAGCAAGCCCAACATTACTCCCTTTGAGTTTGGAAGGTCTACTAATCTTGGGCT
TAAGCCTAACCCTTTCTCTGATACAATGGAAAATCCTCATGACCATATATGATACTTAAAGAGT
TTGTGGGTTTCCCTAGGCTCACCTTGCCAACATGTGGCCAGATGCTGAAAAAGCCGCCTGGGGA
AGTGTGTCATTGATATTGAACAGGAGATTCAGTCTATATCAAAGTGTTCTGATGACAGACTGTG
ACCACACTGGACAGAACCCTATCAAGTGTGGTTGAGAATTCATAGTGCTATCAAGATGAAGGAG
AAACCCAAGTGGATACACGCTTCACATGTGAGACCAACATCCACCATCGAGTGGGGTATAATTC
CCATTTAGGACCTTAAGGATAAATTCTCTAGATAGTAACCTTGAATAATATGCAAGCTAAAGAG
AAAAACTTATTTGATAGACAGCAGGAGAGATAGACTGTTTTGTTATTAATACGTTCTTCTTAAT
TATTTTGACCATTATAATTTTGATACTTAGTTTGTATTAATTTTGATACTTAGTTTGTATCAAT
TGAACATAAAGCTTATTATTAGTCTTTCTTTTAAAAGTTAGTATCTCAACCTCAAGGAATTTTG
TGGTAATTAAAAATTGTCCTGAATATGAGATCAGGACTTAAGAAACCCCTTTCTTTGTCATAAT
ACTTCAAACTCACCAACTGGATGATTTAAATTCAGAAGACATGAAAAATTTCAAATCTTCCCAC
TGATTAGTCAATATTACAAAAATCTTAAATATCATCACCCTTTGTGTTATTGAAGATACTGATG
AGAAGAAATTTTTAACTAATTAAGTTGAACGTAATTATATTCAAATTGAAAGTCCCTGAAGGGT
ATCACAAAACTCCAACAACTCTGTATCTTTTTTTTTTTTGAGCAGAGTTTCACCTTATTGCCCA
GGCTAGAGTGCAATGGCACGATCTTGGCTCACCACAACCTCTGCCTCCCAGGTTCAAGCGATTC
TCCTGCCTCAGCCTCCTGAGTAGCTGGGATTATAGACATGCACCACCATGCCTGGCTAATTTTG
TATTTTTTGTAGAGATGGGGTTTCTCCATGTTGGTCAGGATGGTCTCAAACTTCCAACCTCAGG
TGATCCACCCGCCTTTGACTCCCAAAGTGCTGGGATTACAGGTGTAAGCCACCACGCCCGGCCC
AACTCTAGATCTTAAATAGCCATGGGGGTCCAATTTAATAAAGCTTAAATAGACACACAGATGA
GAAAATATCCCAGCCCAAATGGTGAGGGTTAGTTTCTTCAAAACCCCCAAAACGTATCATCACC
TAAAAATGAGCTTACCCTGTTTAGAACCTGGCAGTCTCCAGTCCCCATACTTACAAACTCGAGG
ATAATTAAATGGTCTGTATCATTTAATATTACAATAGCTAATGTGAGTAAACTGAATTCTACTT
GTACATGATAGGCTTTACCACTATGAAATGTTTTTACACAGGGACTAAAATGGCACTATGATCA
AACTGAAAGAGATTTCTTTTTTTAATAGATGGGGTCTGGCTTTGTTGCCTACACTGGAGTGCAG
TAGCACAATCATAGCTGTCTATCGCCTGTAACTCCTGGGCTCAAGGGCTTCCTCCGTCTCAGCT
TCCTGAGTAGCTGGGACTACAGGGACATGCCACCATGCCTGCCTATTTTTTTTTGTAGAGTCAG
GGTCTTGCTCAGGCTGGTTTCGAACTTCTGGCCTCAAGTGATCCTGCTGCCTCGGCCTTCCGAA
GCACTGGGTTCGCAGACATAAGCCACCATACCTGGCCAGAGATTATTAAATACAGTATTAGGGG
GAATAAAACTAGGACTACAATTGTAAACTCTTCTGATATAGAAACAATTACTGATAAACAGTGC
ATTGGACAATTTGCAGAAAGATGCAAACATGATAGAATCAAAATCTAAACAAGATATGACAAAG
AAAAACAAAAATGGAATTGGGAAAGCATTTGGAATTGGACATCTGGTAATAAAATTCTATTGCA
AAAAAATATATAGTAAAGAATTTTCAGAAGCCTTCACTTGCATTAGTGCACAACCACTGGTTAT
TCAACAACTACAATGTATACTTGCTGGTACTGAAGTACAAGATTATACCTACTCAGACAAACAA
TTAGGTATTGGACATACTACTGTCTATGTAGAGATTAAATGATAGTGTAAAAAGGTTGATCATA
TTATAGGTAAAAGAGCTGAAATCTGGCATTGCAGATTGAAATTTCTGAAAACTGAAATAATAAA
ACTAATTTCACTAAAGTATGTACATTTTCTTATTTACATAGTTTATAATGTGTTATATAATTCA
TGAATCTTCAAAAGAAATATACTGAGCTATTCAATAAAAATTTACATTACAGCTTTAGGCCACC
AAGCCCCATCTATTGATTATGTACTTGGTGTAAATTGGCGGGGGGAACAAAACAAGAGTGAAAA
CTGAGCCTCAAAATACAAAGCATGTAGCAAAACAGACTACTAAAATTATTCTCAATTTCTAAGC
CCATTATATGATGCAGGAGAATTCTTATGTGTAGGTAATAAAGAATCTGTTCAAATATGCATCA
GTGGTATGACAGTTTCAAATAACCCACTGAACTAGGAAATATTCACCACCAAGGAGGTCAGGTA
TACTGATTTGGTCAACCTGGATATTGACATGGGATGTTCTTACGGATTGACAGCTCCACTTGAT
GCTATCCTATCACAAAATATTATTACCTGATGTTGATATAATTAACAAGTCAATACAAAAAGTA
AGGTCTCTCTACAAGACAAGACTAACATCTCTGTAATATAAATGGGACTCTGAGTGTAATAGAT
TTAGGGTTTCTGCAAGAATTTTGAAAGTTAAGGATCCAACGCCTGTAATCCCAGCACTTTGGGA
GGCCGAGACGGGTGGATCACGAGGTCAAGAGATTGAGACCATCCTGGCCAACATGGTGAAACCC
CGTCTCTAATAAAAATACAAAAAAAAAAAAAAAATAGCTGGACATGGTGGCACACACCTGTAAT
CCCCGCTACTCGGGAGGCTGAGGCAGGAGAATCGCGTGAACCAGGGAGGCAGAGGTTGCAGTGA
GCCGAGATCGTACCACTGCACTCTGGCCTGGCAACAGAGCGAGACTCTGTCTCAAAAAAACAGG
AAAGCAAAAGAAAAAGTTAAGGATCCTACCAACAAAATTGGAATATGAGACTGAGGAAATTCCT
ATTCCAGCTAAACCCAGAATTGATTATGTTGATGAACACATGAATTTCATGTGTGTGAAAACAT
ATGAACTCCTCATTGTCAGTCTAATGAGAGTGAAAACAACACATCGAAGATGCGGGGAGACGAC
AATACAGAGTGGGCTCATAACTGCATTCTAGATACTGGGATTACTTTCCATTAAGGTATATAAT
TTTAGAATATCTCACAAATAGCCCATGCAAAATCCTGCGGAAAGGCAACCATAAATATAGAGAG
AATATGTAAAAAAGTGGCATGGTCTAGTATATGCCAAACAAGGCAAGGGTATACAAAAACCTCA
AGGTCCCAGTATGACAAATCATGCTTGTATTGCACACCCATTAAAAAGTCTTCTAAAAAGTTTT
TTTAATGGTGTGCAGGCAGGTTTATTCAGAAAAAGCTCGGATTCATACTTGTAAAAGTCTGAGA
AAAGCATTAAAATGCTATTATGATACAGTTGACCCAAACAACACAGGTTTGAGCTGTGCAGGTC
CACTTATCTGTGGATTTTCTTCTACCTCTGCCACTGAGACAGAAAGACAAACTCCTCCTGCTCC
TCAGACTACTCAGCATGAAGATGAGGATGAAGACCTTTAGGATGATTCACTTCTTCCTAATGAA
TAGTGAATGTATTACTTCCTTAATAACGTTTTTTTTTCTCTAGCTTCTTGTAAGAATACAGTAT
ATAATATATGCAACACAAAAAATACGTGTTAATCAACTGCTTTATGTTATCAGTAAGGCTTCCA
GTCAATGGGAGACTAACTACTAATTAAGTTTTTGGGGAGTCAAGTTATATGCAGATTCTCCACT
GAGCAAATGTTCAGCACTCTTAACTCCTGTGGTGTTCAAGTGTCAACTGTATATATGTTTAAGA
GTTATTGTTTACCCAGTAGATCAAGCCAAGACCAAGTTAGGTTTATGTGTTTTTGGCTTAACCT
AAGAAAATGTTCAGACTAAAGCAGTCCATCAATAGAACAGACTGCCTTTTAAACCAGGGAACAA
CTTGTCTTTGTAGGTATTTTAGGTATATGAATGACCATATGTGATCTGCTTTGGGTCATCTATG
GTCCCTTCCAACTTAAAACTTCTCTATTCATGAACATTGCATAACTGAATTGTGGATTTCATAT
AAATAATATCTTGTTGCCTACTTTAGGTTTGAAATGTTTTCCTTCTCAGTTGCATAGATATGTA
TAGCACCATCCCCACTATGAACTCTCATGTAGAAAATACCAGATTCTAAAGTCATCTGTGAGGC
TAAATGTCAGACTTAGGCACTTCCACATAATGCTGCAAAAAGCAACAGCTTAAAGGTATTACTT
GCTCAACATGTCATTCCAAACACATACACTATGATTAGCAGCTGAATTTAACAGTATCTTTCCA
CTTTGAAAAACACTTTATATACCTTTACAAAGAAGCCATAGTATCTCAGGGTAGAAAAACTCAA
CAGGTAGAAAACATGGCCTTCTCTTAGAAATGAGCTACGTAATTTTGAGTACTTATCTCAAAAT
CATTGCAGAAACATATCTAGTGCTTATGGAAAAAGAATCAAAAGATCTTTTTTTTTAGCTAAAG
ATTTTGAGTTTTTCTTGTGACATTTAAGTCTTTTAATGTATGCTGATTTTTATGATTATCTAAC
TGTTTCATAATGGTTATTCTTGCCTCCATGAAAGGGAAGACTAGAACAAAAACCTGACAAGAAC
AAGTTCTAGCTCCATCACCGACTTACTGAGTGACCCTAGGCAAATCATGTGGGCTCTTTCTTGT
GTTTTAAGTTTTACCTCCAATGAAGTAACTGCCTCAGACTAGCTGTGAAGCTCTCATAAAGTGA
TGGATTTGAAAATGCCTTGCAAAGAATGAGCAGTTAATACATAGAAAGTTTCATGTACTTCCGT
ATATTGCATGATGCCCACCCAGGGTTAGGTTGATAACAGTTGTTGATCCAATAATGAAAGAAAT
AAAAAGAACTCTTTAAAATAACGAAGTAAAATAGCTAATTTTCCTGTTGTAGTTTTAAAAATTA
TATAAACTATATAACTAAACATATTTATGTTTATTATATATAATATGATATGCAATTACATAAG
TATACTTTCCTTCCCCAGATAAGACAAAGAACCTGTTTACTTCTCTGAGGGATTTATTGGTTCC
TGTGAAATCTGTGGAGGCCGCTAACACTCCATAGCCAGAGAATGACAACATATGATTTTCTTTT
CAGTCTTGTAGTATCCACAGTAGTGATGTCTGTCCATGTACAAGTGTCTGTCCAGGACATCCAT
TAAATTCCATGCCTGCTCTGTGTGTTATGAGGGCAGGGACAGAAGACAGATTCACTGGTGGTAC
TTTCAAACAATGTGGTAGGCCTTCCTTTTGGGGTCTGCCATGACAATGATACCCCAGGCGGCAA
TGCTAAGGCCCATGACCATCTTCATAATGGTCCATGGACCCGGGGGGTATCCCATCTTCATGGG
CTTCTGTGCCACCCAGTACACATACCCGCCTGCCCCCATCAGCCGCAACCCAGAAAGCACGTGA
CAGCTCCAGCAGGTCTCCAACAGGTGGTGTTCTGCTGGTGAGGTCGGCGATCCGGGTGTAGCTG
GGGTTGCAGGCTTGGCAGGCACGGCGGCGGTATCGGGAGGCACAGCGATCTTGAGGGACTCAAA
AGGCTGGGACAACTGAGACCCCGTGTTCTGGAACTCGGCCTCCCTAAAAATTATGTTTTAAAAA
ATACATAGAATCGGTAAGATGAGGAAAAAGCATAAAACGGTCAAAAACACTGGGATAATTCAAG
GTTCATTTTGACAATGGTAATACAAATAGAAAAACTGACAAAAAAGAAAATACATCTTACATTA
GAAAACATACAAGAGTAGTTGTATACAAACGAAGACACGTACCAAAAATAAGTTACAAAATATG
AATGCATATACATAAAAAATGTAGTTTCAAACAATTCAAAAGTAAAATGATGCAAGACATTAAA
ATATTCACAAACACGCTCGAAAAATAAGAGCTGACGCCCAAATTAGACCAGTTCAACGGAAGTG
TGGGGTGTGGTTGGGGATCCGCCGCAGAAATCACGTGGCGCCCAGGCCAAGGCTGCAATTTCCC
ATGCTGCTTGGAGAAGACGCCTGGGAGGGCTCCCACCATGCCCAGGGGCAGGCTATGTGACTGC
CCGGTCTGCAGCTGTAAGTGGTTTCTGGGCCAGTCTCTTTGTCTGGTCATGACCTGGTTGGTGG
CCCTAGCTTGTGCCGAGTGGTCCCGGAGTGGCCCAGGGGGTTGTGTGTGGGGAGCCAGCGTGGG
CAGCGGAGGTGCTCCCTGGGGCCTGCAGTAGGGCACAGCCTCTGCATCTTGTGTGAGCCCAGTG
ACCCGCCTGTCCTGTCCTTCCAGGGTTGGGTGGCAGGGAGGGCCAAGGGTGCCCTGCGTCCGTG
ACGGGTCAGCGTTTCCAGCAAATGGGTCCATGGAGGGAGAGCCTAGCTGAGCCGGCGGGCTCTA
CCACCTTCATGAGGTTCTGTTTGGACACCTGTCCTACTCCTGTTCCCCCAGATGGTGACCATGG
AGGAAGACTTGTGTTGGGCCCAATGGCCCTCGGCCAGTGTCCGTCTGCCAGGTGCCAGCCCCCG
GTGCGCTGAAGCTCAGGCCATTCCTGATGCCCTGGCCTCCTGCACTGAGCTGTGGTGAGCCCAT
CCGGGTCCTGCTGGATACATGGGCGGGGAGGGGGGTGCCCTGGGTTGTGTTGATGATGAGAACC
TTATATTATCCTGAAGAGAGGTGATGACTTAAAAATCATGCTCAATAGGATTACGCTGAGGCCC
AGCCTAGGTGAGAATTTTGGAAGAGGATGCTGGGATCCAGAGGCCCCCGACGGGGCCACTGTAT
TTCGGGCTGCAGACCTAGAGGCCCTGAAGGGCATCTGAAGGTGGCCCAAGCCTATCTCTGAGCA
ACCCCGTGAGGCAGTCAAGGATCCCTGTGTGGGCATAGGCTGGGGCTCTGGTTCCCTAGGGGCA
GGGCATGTGGGTGGCTCCTCCCTGACCCCCATTCTCCCCTGTGTCTTTCAGTGAGCTCTTCTGC
CCAGGCAGGCCCCCTGGCATTGACCGGCATAGGTGAGTGGATCCTGCTGGGGTCATGGGCCATG
AGCCAGGCTGCTGGGGTCGCCAAGGGTCAGCCTTTGGGACAGGAGGGTTTCCTTAGGGAGCCAA
CCTGAATCCCCACTGGGGAGGGCTAGGTTCTCCAGGAGGGCAGCTTCCTTGGGAGTCTGACCCA
AGGAGGATATCTTCTCTAAAGCCCTGCAGTGGTGAGGGTGGTCCAGGCAGCAAAGGGTTCCTTC
AACCCCACCCAGGACAGCGCCCAGCTGAGACTCAGTGTGCAGGTCTGGCCATGAGGACTGCCCA
GGTCACATAAGGGGGTCCATAGGAGCTCCGGGGCACCACAGGGGTCACACAGGAGCTCTCCTTC
CCCAGAGAGGGGTGAGCGATTGGGCCTATGGGGTCAGGCCTTTGGAGATCAAGGATTGGGCCCC
ACCCATGAACCTGAATGGGACTGAGCCTTTGAGCAATGGACCATCCCAGTGGTGCCTCGGAGCC
TAGTCGGGGGTGATGTGGCTTCAAGGGGGTTGGGTCAAGGCTGAGCTCTGGCCGGTGCCTGGTA
GTGCTGCAGGCTGGCCCTGGGACTCGCTGGCTTTGGGCAAAGGGGCCAGCCCGTGTCCCACTGG
AGGTCACCCCTTCCCTGTGGGCAACTAGCAGGAAGGGTGGCATCCTTAGCTGGGAAGGCCCACT
GTCGAGGGTCCTGTCCAGGGAATTTGCCTCCCAGGCTTCTTCTGATCCACGGACTCACCTCCAG
GGTGGTGGGGTCTTGGGCCCGAGGAAAGGAGAAGATACCCTGTGTTCCCCAGTTGGCTGAGTGT
GGCTTGGCTCTGTGTTTGGGAGGTGTGTAGGGTATGAATGGGGACCCACCAGAGAACACACATG
GCCCAGGGCCCCAGGTTATGATTTCCCATGCACACTTGGAGAGAACTCTGGGTGAGTGCCCTCC
ACTCCCAGGCACAGGCTATGCAACTGCCAGGTCTGCAGTGGCATGTGGCTTCCAGGTCAGCCTC
CTTGGTTGGCCATGAGCAGGTTGGTTGCCCTGTCCTGTGCCCAGTGGTCTTGGAGTGGCCCAGG
GGTCCTGCAGGGGAGCTGGTGTGGGTAGTGGAGGTGCTTGTGGGGCCTGCAATAGAGCGCAGCC
TAGGTGCCTTGCACGAGCTCAGTGACCCACCTGCCCTGTCCTAGTCCTAGCTCAGTGACCCACC
CCCGCTTTGCAGTGATGGGGAGTACCAAGCGTCTCCTGCGTCTGTGATGGGTCAGTGTTCCCAT
CACATGGGTCCATGGAGGGAGAGCATGCATGAGCTGGCACCACCCACCACCTTCCCACTATGGA
GGCGTGAGGCCATGTTTGGACACATGTCTTCTTGTCCCCCCAGACAGTGAGCCTGGAGGAAGAC
TTGCCCTGGTCCCACTGTCCCTGGGCCAGTGTCCATCAGCCAGGTGCCCAGACCCCAGTGCGCT
GAAGCTCAGCCCTCCCTGGCACTCTGGTCTCCTGCACTGAGCTGTGGTGAGCACATCCAGGTCC
TGCTGGATGTTTGCATGGGGAGGGGCTTTCCCCGGGTTGGGTCGATGATGAGAAGCTTCTGTTT
TCTTGAAGAGAGGTGATGACTTAAAAATCATGCTCAATAGGATTATGCTGAGGCCCAGCCTAGG
TGAGAATTTTGGAAGAGGATGCTGGGATCCCCGGCAGGAGGGATTTTGGGCTGGAGACCTGGAG
GCCCTGAAGGGTATCTGCAGGGGCCCCATCCCTGTCTCTGCACTTGTCTGTGAGGCAGCCCAGT
TTCCCTGTGTGGGCTTCGTGTCAGTGCTCTGGTTCCCTGGAGCAGGGCACGTGGGTGGCTCATC
CCTGAAGTCACGTTCTCCCTTTGTGTCTTTCAGTGAGCTCTTCCGCCCATGAGGGCCCCCTGGC
CTTGAGCAGCATAGGTGAGCGGATCCTGCTGGGCTCATGGACCAGGCCACATGGGGTCACTGAG
GGTCAGCCTTCAGGACAGGAGGGATTCCTCAGGGAGACAACCTGAATCCCCACTGGTGAGGGAT
TGGTTCTCCAGGAGAACCACCTCCTTGGGAATCTGACCCAAGGAGGACACCTTCCCTGAAGCCC
TGCAGGAGTGACAGTGGTCCAGGCAGGAAAGGGTTCCATTAGTCCCATGCAGGGCAGCTCCCAG
CTGAGACTTGGTGCTAGGGTCTGGCCTCGAGGACTGCCCAGGCCACATGAGGCAGGGTCTCAGG
CTGCTATTTCCAGTGCATGCTTGGAGAGGGAGCCTGGGTAGGCCCCCTCCCTGTGCAGGGGCAG
GCTGTGCATCTGCTGGGGCTGCAGCGGTGCCTGCCTTCAGGACCAGCCTCCTTGGTTGGCTTGA
GCTGGATAGTGGCCTTGGCCCCACCCAGGGGTCATGGTGCAGCCCAGGAATTGCAGGGGAGCTG
GCATGGGGAGTGGAGGTGCTCCAGGGGCTTGCAGTAGGGCACAGCCTGGGCATCTTGCTTGAGC
CCAGTGACCAGCCTGCCCTGTTTTCCAGGGTTCAGTGGCAGGGAGGACCAAGGGTCCCCCACGT
CCATGACTATCACTGTTTCACATGGATCCCATCAGGTGGGTCCATGGAGGGAGAGCATGTGTGA
GTCAGCACCCCCTGCCTCCTTTCCCGCATGGAGGCATGAGGTTCTGTTCGGACACTTTTCCCTG
TCTCTCCAGATGGTGACCCTGAAGGAGGACTTGTGTTGGGCCCAATGGCCTGGGGCCAGTGTCT
GTCAGCCAGGTGCCCAGCCCCTGGCGTGCTGAAGTTTGGGCCCTTCCTGGTGCCCTGTTCTATC
GGGGGAACCCGCCCCCAATAATTCAACGTAGGTCCTTTTCTATTTTCCCTAAGTGTCGGCCGGT
CTGAGAAATAAAGGGAAAGAGTACAAAAGAAAGAAATTTTAAAGCTGGGTGTCCGGGGGAGACA
TCACATGTCGGCAGGTTCCATGATGCCACCCAAGCTGCAAAACCAGCAAGTTTTTATTAGTGAT
TTTCAAAGGGGAGGGAGTGTACAAATAGGGTGTGGGTCACAGAAATCACATGCTTCACAAGGCA
ATAAAATATCAGAAGGCAAATGAGGGCAGAGCACCAGGGCCAAATTGAAATTGCTAATGAAGTT
TCGGGCACGCATTGTCATTGATAACATCTTATCAGGAGACAGGGTTTGAGAGCAGACAACCGGT
CTGACCAAAATTTATTAGGCAGGAATTTCCTCGTCCTAATAGGCCTAGGAGCGCTTCGGGAGAC
CAGGGCTTATTTCATCCCTTATCTACAACTGTATAAGACAGACATTCCCAGAGCGGCCATTTTA
GAGACCTCCCCCTAGGAAGGCATTCTCTTTCTCAGGGTTGTTCCTTGCTGAGAAAAATAATTCA
GCAATATTTCTCCTATTCGCTTTTGTAAGAAGAGAAATATGGCTCTGTTCCGCCCGGCTCTCAG
GCAGTCAGGCCTGATGGTTGTCTCCCTTGTTCCCTGAACATCGCTGTTATCCTGTTCTTTTTTC
AAGGTGCCCAGATTTCATATTGTTTAAACACACATGTTTTACGAACAATTTGTGCAGTGAACGC
AATCATCACAGGGTCCTGAGGCGAAATACATCCTCAGCTTACGAAGATGACGGGATTAAGAGAT
TAAAGTAAAGACAGGCATAGGAAATAATAAGAGTATTGATTTGGGAAGTGATAAATGTCCATGA
AATCTTCACAATTTGTGTTCTTCTGCCATGGCTTCAGCAGGTCCCTCCGTTTGGGGTCCCTGAC
TTCCCTCAACACTGGTCTCTTCCACTGAGCCTTGGTGAGCCCATCTAGGTCCTACTGGATGCAT
GTGTGGGGAGGGGGGTGCCCTGGGTTGGGTCAATGATGAGAACCTTATATTGTCCTGAAGAGAG
GTGATGACTTAAAAATCATGCTTAGTAGGATTACGCTGAGGCCTAGCCTAGGTGAGAATGTTGG
AAGAGGATACTGGATGCTGAGGTCCCTGGCTGAGCTACTGTATTTTGGGATGGAGATCTGGTGA
CTCTAAGGGCACCTGAAGGGCCAGACCCTGTCTCTATGAGGCAGCCCAGGCTCCCTGTGCAGGC
TTGCTGTTGGCACTCTGGTTCCCTGGGGTGGGACATATTGGTGGCTCCTCCCTGAGCCCTGTTC
TCCCCTTGTGTCTTTCAGTGAGCTCTTCCACCAAGGAGGGCCCCCTGGCATTGACTGGCATAGG
TGAGTGGATCCTGCTGGTGTCATGGGCCATGGGCCAGGCCACGTGGGGTCACCAAGGGTCAGCC
TTCAGGAAAGAAGGGTTTCCTCAGGGAGCCAAACTGAATCCCCACTGGGAAGAGATGGGTTCTC
CAGGAGGGCGGCTTCCTGGGAAACAGGAACCAAGGAGGACGCCTTCCCTGAAGCCCTGCAGGGG
TGACGGTGGTCCAGGCAGGAAAGGGTTCCTTCAATTCCACCCAGGGCAGCTCCCAGCTGAGTCT
CGGTGCTCGCGTCTGGACTGCCCTGACTATGTGGGGGGCCACAGGGGCTCTGGGGTGCCTTGGG
GGTCACACAGGTGCTCTTTGTCCCTGGAGAAAGGTGGGTGATCAGGCCCATGGAGTTGAGCAGT
GGAGATTCAAGGATTGCACCTGACACATGGTCCTGAATGGAACCCACCCTGTTGAGCAATGGGC
CATCCCATGGGCCTCTTCAAGCCTGAAGAGGGGCGGTGCTGTTTCACTGGGTTTAGGTCAAGGC
TGAGCTCTGGCCGGTGCCTGGTGGTGCTGCAGTCTAGCTGTTGGACTCGTGGGCTTTGGGCCAG
TGGGGCAGCCTGCATCCCTCTGGAAGTCACCCCTTCCCTGTGGCTGGAAGTTACCCCTTCCCAG
TGGGCGAATGTGGGGAAGGGTCACCTCCTGAAGTGAGAAGGCCCACTATTGAGGGTCCTGTCCT
GGCAATTTGCTGCCCATGCTGCTTCTGAGCCATGGAGACACCTCCAGTGTGGTGGGATCTTGGG
CCCAAGGAAGGGAGAAGACACTGTGCCCCCCGATTAGCTGAGTGTGGCCAGGCTGTGTGTTTAT
GAGGTGTGAGGGGCATGGATGGAAACTCACCAGAGAAAGCATGTGGCCCTGGGACCCCAGGTCG
TGGTTTCCAGTATGTGCCCAGATACAGCACATGGGTGGGTGCCCACCATGCACAGGGACTGGCT
ATGTGACTGCCAGCTGTGCAGCAGCATCAGGCATATGGGCCAGCCTCCTTGGTTGACAATGAAC
TGGTTGCTGGCCCTGGCCTGTGCCCAGTAGTCCTGGGGTGACCCTGGTGTCATACAGGGGAGCT
GGCATGTGTAGTGGAGGTGCTTGTGGGGCTTGCAGTAGGGCACAGCCCTGGCTTCTAGCCTGAG
CCCAGTGACCTGCCTGTCCTATCCTTTCGGTGTTCAGTGGCGGAGAGAGCATTGGGTGCCCTGC
ATCCATGATGGGTAAGTATTCCACATGGATCTCATCACATGGGTCCATGGAAGGTGAGCATCCC
TGAGCTGGCACACCACACCTTCTGTCCACATGGAGGGGTGAGGTTGTGTTTGGGCACTTGTCCT
CTTCCTGTTCCTTGAGATGGTGACCCCAGAGGAGGACATGCGTTGGGCCCGCTGTCCCTGGCCA
GTGGCCATCAGCCAAGTGCCTAGCCCCTATTGCACTGAAACTCGGGCCCTTCCTGTCACCTTGG
TCTCCTGCGTTGAGCTTGGGTGAGCCCATCCGGGTGCCACTGGATGCCTAGGCAGGGAGGGGGG
TGCCCTGGGTTGGGTCGATGATGAGAACTTTATATTGTTCTGAAGAGAGGTGATGACTTAAAAA
TCATGCTCAATAGGATTACGCTGAGGCCCAGCCTAGGTGAGAATTTTGGAAGGGGACACTAGAA
TCCCGAGGTCCCCAGCAGGGCTGCTGTATTTTAGGCTGGATACCTCGAGGCCCTGAAGGGCACC
TCATGGAGGGGCCCAATCCTGTCTGTGCTCCTCTATTAGGCACACCAGACTTTCTGTGTGGGCT
TGGTGTCCACGCTCAGCTTCCCTGGGGTGGGACACGTTGGCGGCTCCTCCGTGAGCCCCATTAT
CCTCTGTGTCTTTCAGTGAGCTCTTCCCCCCAGCAGACCCCTGGGATTGACGAGCATAGGTGAG
TGGAGACTGCTGGTGTCATGGGCCATGAGCCAAGCTGCGTGGGGTCCCCAAGGGTCAGCCCTCG
GGCCACCTGGGTTTCCTCAGGAGCTGATCTGAATCCCAACTGGGGATGTGTTCTCCAGGAGGGC
AGCTTCCTTGGGAATCAGACATAAGGAGGAGGCCTTCTCTGAAGCCCCGCAGGGGTGGCAGTTG
TCCAGGCAGGAAAGGGTTTCCTCAGCCCCACGCAGGGCAGTGCTCAGCTCAGACTTGGTGCTTG
GGTCAGGCCTCTAACATGGCCAGGCCACATGAGGGGGGCAACAAGGGCTCCGGGGAGCCAAGGG
CATCTCACGGGGGCTCTCCAACCCTGGAGAAGGGTGGGTGATTCGGTCCATGGGGTCAGGCCAT
GGAGATTCATGGATTGCAGCAAACCCAGGACCCTCAATGGGACTGACCCTATAAGCAGTGGGCC
CTCCCAGTGGCACCTCTTTGAGCCATTTGTGGGGCGGCGCTGTTTCTCTGAGGTTGGGTCAAGA
CTAGCTCTGGCCAGTGCCTGATGGTGGTGCAATCCACTCCTGGGACTCGTGGGCTCTGGGCCAA
GGCTGCAGCCCATGTCCCGCTGGGGGTCACCCCTTCATTGTGGGCGAGTGGCAGTAATGGCCTC
CTGAGTTGGGAAGGCCCGCAGTGGAAGGACCTTTCCTGGAAATTTGCAGTCCAGGCTGCTTCTC
AGCCATTAGGACAACTCCAGCGGGTTGGGGGGCAGGGGCGGGTCTTAGGCCCAAAGAAAGGAGA
AGATACCATATGTCTCCCGGTTGGCTGAGTGTGGCCCTGCTGTGTGTGTGGGAGGTGTTGTGTG
GGGCATGGATTGGGACACACCAGAGAAAGCTTGTGGCCCTGGAGCCCCAGGCTGCGATTTCCAG
TGCACCTTGAGGAAAGGAGGGTGGGTGCCCTCCATGCACAGGGCAGACTGTGCAACTGCCAGGT
AGGCAACAGCACGTGGTGTCCGGGCCAGGGTCCTTGGTTTGCTGTGAGCTCTTTGGTGTCCGTG
GCCCCTACCCAAGGATCCAGATACCACCCGGGGTCATGCAGGGGAGCTAGCATGGGCAATGGAG
GTGCTCAAGGGGCCTGCAGTAGGGCACAGCCCAGGTGTCTTGCCTGAGCCCAGGAACCCACCTG
CCCTGTCCTTCCATGGTTCAGTGGCTGGGAGAGCAAAGGGTTCCGCTGATCTATGACACATCAG
TGTTTCCCATGGATCCCATCACATGGGTCCACGGAGAGAGAGCATGTGTGAGCTGGCACCCCCA
ACCCCATTCCCTGTATGGAGGCGTGAGCCCATGGTTGGACACATGTCCTCCTCCTGTCCCCCCA
GATGGTGACTGTGGAAGAAGACTTGCTTTGGGACCGCTGGCCCCAGGCCAGTGGTTGTCATCCA
GGTGCCCAGCCCTTGGTGCACTTGAGCTCAAGCCCTTCCTGGTGCCCTGGCCTCCTGCCCTAAG
CTGTGGTGAGCCCATCCAGGTCCCACTGGATGCATGCATGGGGAGGAGGGTGCCCTGGGTTGGA
TCGATGATGAGAACCTTATATTGTCCTGAAGAGAGGTGATGACTTAAAAATCATGCTCAATAGG
ATTACGCTGAGGCCCAGCCTTGGTGAGAATTTTGGAAGAGGATGCTGAGATCCCAAGGTCTCTG
GCAGGGCCACTGTATTTTGGGCTGGAGACCTGGAGGCCCTGAAGGGCATCTTAGGGGTGCCCGA
CCCCATCTCTGCGCTCCTCCTTGAGGCAGCCCAGGCTCCCTTGCGGGCTTGGTGTCAGCCCTCC
AGTTCCCTGGGGTTGAGGCACATTGGAGGCTCCTTCCTGAGCCCCCATTCTCCCCTGTGTCTTT
CAGCGAGCTCTTCTGCCCAGGGGGACCCCTGGCCTTAAGTGGGATAGGTAAGTGGATCCTGCTG
GGGTCGTGGACCATGAGCCAGGTCGCTTTGGGTCACTGAGAGTCACCCTTCAGGCCACGAGGGA
TTCCTCAGGGAGCCCATCTGAAACCCACCAGGGACGGATGGTTTCTCCAGGAGGACAGCTTCCT
TGGAAGTCGGTCCCAAGAAGGATGCCTTCCCTGAAGCCGTGCAGAGATGATGGACGTCCAAGCA
GGAAGGGCTTCCTTCAACTCAATGCAGTGCACATCCTAGCTGAGACTCAGTGCACAAGTCAGGC
CTGGAGGACTTCCCAGGCCACATGAGGGGGTCCACAGGGTCTCCAGGGGCCACGGGTGTTATGA
GGGTGCTCTCTGTCCCCGAGGCTTGCAGAGGGGCAGGGTGTGGGTGATCGGACTTATGGAGTCA
GGCCGTGAAGATTCAAGGATTGGGCCCAACCCATGGCCCAGCATGGGCCGAACCTGTAGAGCAA
TGGGCCATCCCGGTGGTGCATCCTTGAGCCTTGTGGGGGTGGTGTGACCTTAGTGGGGTTGGGT
CAAGCCTGAGCTCTGGCCTTCACCTGGTGGTGCTGCAGGCTGGCCCTGGGAATTTGGGCTTTGG
GCCAAGGGTGCAGTCTGTGTCCCGCTAAAGGTCACCCCTCCCCAAAGTGGAGTGGCCAGAAGGA
TGGCATCCTGTGGTAGAAGTCCCACTGTCAAGGGTCCTGTCCTGGCAATTTGTTGCCCAGGCTG
CTTCTAAGCCATGGGTTCACCTCCAAAGTGGCGGGGTCTTGAGCCGAAGGAAAGGGGAAGACAT
CATGTGTTCCCTGATTGACTGAGCATGGCCTGGCTCTGCATTTGGAGGTGTGCGGTGCACGAAT
GGGGACCCATCAGAGAAGGCATGTGGCCCTGGGGCTCCAGGATTTGATTTCTGGTGTGAGCTTT
TACAGGGGGTCTGTAGTGGCACGTAGCATTCAGGTCAGCCTACTTGGTTTGCTCTGAGCTTGTT
GGTGGTCCTGGCCCTGCCCAGTGGTCCCATTGTGGCATAGAGTCATGTGAGGGAGCTGTGGCAA
GGAGGGCCAAGGGTTCCCCACATTCATGATCGTTACTGTTTACATGGATCCCATCTCTTGAGTC
CATGGAGGGACAGCATGTGTGAGCCAGCACCCCCTGCCCACTTTCCTGCATGGAGGCGTGAGGC
CGTATTTCTATACGTGTCCCCCACCCCTCCAGATGGTGACCCCAGAGGAAGACGTGCATTGGGT
CCGCTGGCCCCAGGCCAGTGTCTCTCAGCCATGTGCTCAACACCTGGTGAGCTGAAGCTCAGGC
CCTTCCTGGCATCCTGGTCTCCTGCACTGAGCTGTGGTGAGCACATCTGGGTTCCTCTGGATAC
GTGTGCAGGGAGGGGGATGCACTGGGTCGGGTCAATGATGAGAACCTTATATTGTTCTGAAGAG
AGGTGATGACTTAAAAATCATGCTCAATAGGATTACGCTGAGGCCCAGCCTAGGTGAGAATTTT
GGAAGAGGATGCTGGGATCCCAAGTTCCCCAGCATGGCCAGTATATTTTAGGCAGGAGACCCTG
AGGCTCTGAACGGCATCTCCGGGGGGCACAACCCTGGCTCTGGGCTCCTCTGGGAGGCAGCCCA
GGTTCCCTGTGCAGGCTTGGGCTCTGGTTCCCTGAGCCCCCATTCTCCCTCGTGTGTTTCAGTG
AGCTCGTCCTCCTAGGTGGGGCCCCCAGGCATTGACCGGCATAGGTGAGTGGTCCTGCTGGGGT
CATGGGTCATCAGCAGGTTGGAGTCACCAAGGGTCAGCCTTTGAGACAGGAGGGTTTCCACAGG
CAGCCGACCTGAATCCACACCATGGAGGGATAGATTGGTCCTCCAAGAGGGCTGTTTTTTTTCG
GAATCAGACCCAAGGAGGATACCTTCCCTGCAGCCCTGCGGGGATGACGGTGGTCCAGGCAGGG
AAGGGTTCCTTCAGTCTCACGCAGGTCAGCTCCCAGCTGAGTCTCAGTGCATTGGTCTGGCCTC
CAGGACTGACCAGGCCACGTGAGGGGGGCAACAGGGGCTTCGGGATGCAGTGGGGTCACACAGG
ATCTCTCCATCCCCAGAGAAAGGTGGGCATAGAACCCATGGATTCGAGCTGCAGAGATTCAAGA
ATCGCACACGAATTATTGCCCTGAATGGGACCCACCCTGTTGAGCAATGTCCCATCCCAGGGCA
CCTCTTTGAGCCTGAAGGGGGTGGTGCTGTTTCCCTAGGTTTGGGTCAAGGCTGAGCTCTGGCC
AGTGCCTGGTGGTGCTGTAGTCCTGTCATGGACTCGTTAGCTTTACGCCAAGGGGTCAGCCCAC
GTCCTACTGGGGATCTCCCCTTCCCTGTGGCTGGAAGTCACCCCTTCCCCATTGGCAAGTGTCA
GGAAGGGTGACCTCCTGAGGTGGAAGTCCTGTTGAGGGTCTTGTCCCTGGAATATGCCACGCAT
GCTGCTTCTGAGCCATGGGGTCACCTCCAGGGTAGCGGGTCTTGGGCCCAAGGAAAGGAGAAGT
CACCATGTGTCCCCCGATTAGCCAAGTGCGGCCCTGCTCTGTGTTTAGGAGGTGTGTGGGGCAT
GGATGGGGACCCACCAGAGAAAGCATGTGGCCCAGGGACCCCAGGTTGCAATTTCTAGGACATG
CTGGGATAGGGCACCTGTGTGGGTGTCCACAGTGCACAGGGATAGGCTATGTGACTTGCAGGTC
TGCAGCGGCATCTGGCATCCAGGCCAGCCTCCTTGGTGGGCCGTGAACTGAATGGTGGCGCTGG
CCTGTGCCCAGTATATCTGGTATGACCCTGGGGTCGTGTGGGGGAGCCGGCATGGGCAGTGGAG
GTGCTTATGGGACCTGCAGTAGGACACAGTCGGGGCTTCTTGCCCAAGCCCAGTGACCCGCTTG
CCCTGTCCTTCCAGGGTTCGGTGGCTGAGAGAGCTTTTGTTGCCCTGTGTCCGTGACGGGCCAG
TGTTCTGCATGCATCCCATCACATGGATCCATGGAGGGTGAGCATCCCTGGGCTGACACACCCC
ACCTTCTGCCCCCATGGAGGGGTGAGGTTGTGTTTTCACACCTGTCTTACTCCTGTTTCCCCAG
ATGTTGAGCCCAGAGGAAGACTTGCACTGGGCCCAATGGCCCCAGGTCAGCGTCCATCAGCCAG
GTGTCCAGCCCCTGGTGCACTGAAGATCGGGCCCTTCCTGGTGCCCTGGTCTCCTGCATTGAGC
TGTGGTGAGCACATCCGGATCCTGCTGGATGCGTGTGCGGGGAAGGGGGTGCCCTGGGTTGGGT
CAATGAGAACCTTATATTGTCCTGAAGAGAGGTGATAACTTAAAAATCATGCTCAATAATAGGA
TTACGCTGAGGCCCAGTCTAGGTGAGAGGTTTGGAAGAGGATGCTGGGATTCCGAGGTCCCCAG
GAGGGCCCCTGTATTTTTGGCTGGAGACCTGGAGGCCCTGAAGGGCATCTTGTGGGTGCCCAAC
CCTGTCTCTGCGCTCCTCCTTGAGGCAGCCCAGGCTCCCTGTGCAGGCTTGGTGTCAGCCCTCC
AGTTCCCTGGGTTTGAGGCACATGGGAAGCTCCTTTCTGAGCCCCGTTCTCCTCTTGTGTCTTT
CAGTGAGCTCTTCCACATGGAAGACCCCTGTCATTGACTGGCATAGGTGAGTGGCTGGCATCAT
GGGCCATGAGCCAGGCCGCGTGAGGTCGCCAAGGGTCAGCCTTCGGGAAAGAAGGGTTTCCTCA
GGGAGCCAACCTGAATCCCCACTGGGAAGATATGGGTTCTCCAGGAGGGCGACTTCCTGGGAAA
CAAGAACCAAGGAGGTCCCCTTCCCTGAAGCCCTGCAGGGGTGACGGTGGTCCAGGCAGGAAAG
GGTTCCTTCAGTTCCACCCAGGGCAGCTCCCAGCTGAGGCTCGGTGCTTGCGTCTGGACTTAAG
GACTGCCCTGGCCATATGAGGGGTGCCACAGTGGCTCTGGGGTGCCATGGGGTCACACAGGCAC
TCTCTGTCCCTGGAGAAAGGTGGGTGATTGGGCCCATGGAGTTGAGCTGTTGAGATGCAAGTTT
TGCACCTGACACATGGCCCTGAATGGGACCCACCCTGTTGAGCAATGGGCCATCCCATGGGCCT
CTTCGAGCCTGAAGGGGGGCGGTGCTGTTTCACTGGGTTTAGGTCAAAGCTGACCTCTGGCCGC
CACCTGGTGGTGCTGCAGTCCAGCCCTTGGACTCGTGGGCTTTGGGCCAAGGGGGCAGCCTGCA
TCCCACTGAAAGTCACCCCTTCCCTGTGGCTGGAAGTTATCCCTTCCTCATGGGCGAGTGTTGG
GAAGGGTGGCCTCCTGAAGTGGGAAGGCCCGCTATTGAGGGTCTGCTCCTGGCAATTTGCTGCC
CATGCTGCTTCTGAGCCATGGAGTCACCACCAGTGTGGTGGGGTCTTGGACCCGAAGAAAGGAG
AAGACACTGTGCCCCCCGATTAGCTGAGTGTGGCCAGGCTGTGTGTTTGCGAGGTGTGAAGGGC
ATGGATGGAGACTCACCAGAGAAAGCATGTGGCCCTGGGGCCCCAGGTTGTGGTTTCCAGTATG
TGCTTGGATATGGCATATGGGTGGGTGCCTACCATGCACAGGGACTGGCTATGTGACTGCCAGC
TGTGCAGCAGCATCAGGCATACGGGCCAGCCTCCTTGGTTGACCATGAACTGGTTGCTGGCCCT
GGCCTGTGCCCAGTAGTCCTGGGGTGACCCTGGTGTCATGTAGGGGAGCTGGCATGTGTAGTGG
AGGTGCTCGTGTGGTCTGCAGTAGGGAGCAGCCCTGGCTTCTAGCCTGAGCCCAGTGACCTGCC
TGTCCTGTCCTTTCAGGGTTCAGTGGCGGAGAGAGCATCAGGTGGCCCACGTCTGTGATGGGCC
AGTATTCCACATGGATCTCATCACATGGGTCCATGGAAGGCGAGCATCCCTGAGCTGGCACACC
CCACCTTCTGCCAGCATGGAAGGGTGAGGTTGCATTTGGCACCTGTCCTCTTCCTGTTCCTCGA
GATGGTGACCCCAGAGGAGGACTTGCATTGGGCCCACTGTCCCCGGGCCAGTGGCCATCAGCCG
GGTGCCTAGCCCCTGTTGCACTGAAGCTCAGGCCCTTCCTGTTGCCCTGGTCTCCTGCATTGAG
CTTGGGTGAGCCCATCCGGTTGCCACTGGATGCCTAAGCAGGGAGGGGGGTTCCCTGGGTTGGG
TCAATGATGAGAACCTTACATTGTTCTGAAGAGAGATGATGACTTAAAAATCATGCTCAATAGG
ATTACGCTGAGGCCCAGCCTAGGTGAGAATTTTGGAAGAGGACGCTGGGATCCCTAGGTCCCTG
ACAGGGCCACTGTATTTTGGGCTGGAGACCTGGAGTCCTTGAAGGGCATCTGGAGGGGGCCCAA
ACCTGTCTCTACGTTCCTCCATGAGGCAGTCCAGGCTCCCTGTGCGAGCTTCGTGTCGGTGCTC
AGATTCCCCTTGGTGGTGCATGTGGGTGGCTCATCCCTGAGCCCCTGTTCTCCCCTTGTGTCTT
TCAGTGAACTCGTTTGCCCAGGAGGACCCCTGGCCTTGAGCAGCATAGGTCAGTGGATCCTACT
GGGGTCATGGGCCATGGACCAGGCCACGTGGGGTCACTGAGGGTCAGCTATCAGGACAGGAGGG
ATTCCTCAGGGAGACAACCTGAATCCCCACTGGTGAGGGATTGGTTCTCCAGAAGGGCCGCCTC
CTTGGGAATCTGACCCAAGGAGGACACCTTCCCTGAAGTGCTACAGGAGTGACATTGGTCCAGG
CAGGAAAGGGTTCCATTAGTCCCATGCAGGTCAGCTCCCAGCTGAGACTTGGTGCTAGGGTCTG
GCCTTGAGGACTGCCCAAGCCACATGAAGGTGGCCCCAGGCTGTGATTTTTGGTGCGTGCTTGG
AGAGGGAGTCTGGGCACCCTCCCTGCACAGGGGCAGGCTGTGCGTCTGCCAGGTCTACAGTGGC
GCCTAGCTTCAGAGCAAGCTTCCTTGGTTGGCCTTAGCTGGTTGGTGGCCCTGGCTCCTACCCA
GGGGTCACAATGCAGCCCAGGAAGTGCAGGGAAGCCGGCTTGGACAGTGGAGGTGCTCCAGGGG
CCTGCGGTAGGGCACAGCCTCGGCATCTTGCCTGAGCCTAGTTACTCGCCTGTCCTGTCCTTCC
AGGGTTTGGTGGTGGGGAGATACAAGGGTTCCCCAGGTCCATAACTGTCCTCGTTTCACATGGA
TCCCATCACATGAGTCCACGGATGTAGAGCATGTGTAGCCAGCAGCCCCTCCCCGCTTTCCTGC
ATGGAATCTTAAGGCCCTATTTGGACACCTTTCCCCGTCCCTCCAGATGGTGAGCACAGAGGAA
GAATTGCGTTAGGCCCTTTGGCCCCGGACCAGTGTCTGCCAGCCACGTGCTCAGCCTCCCGTGC
GCTAAAGCTCAGGTCCTTCCTGTCATCCTGGTATCCTGCACTGAGGTGTGGTGAGTCCATCCAG
GTCCCTCTGGATGTGTGAGTGGGGATGGGGGTGTCCTGGGTTGGGTCGATGATGAGAACCTTAT
ATTGTCCTGAAGAGAGGTGATGACTTAAAAATCATGCTCAATAGGATTACGCTGAGGCCCAACT
TAGGTGAGAATTTTGAAAGAGGATGCTGGGAATCACAAGGTCCCCGGCAGGACCACTGTATTTT
AGGCGGGAGACCTCGAGGCCCTGAAGGGCATATCATGGAGGGGTACCCAACCCAGTCTGTGCTC
CTCCATTAGGCACACCAGGCTTCCTGTGTGGGCTTGGTGTCTGCACTCAGCTTCCCCGGGGGTG
GGACACATTGGTGGCTCCTTCCTGAGTCGCATTCTCCTCTGTGTCTTTCAGTGAGCTCTTCCCC
CCAGCAGGCCCCCTGGGATTGACTGGCAAAGGTGAGTGGATACTGCTCGTGTCATGGGCTGTGA
GCCAAGCTGCGTGGGGTCCCCAAGGGTCAGCCCTCGGGTCACCTGGGTTTCCTCAGGAGCTGAT
CTGAATCCTGACTGGGGATGTGTTCTCCAGGAGGGCAGCTTCCTTGGGAATCTGACACAAGGAG
GAGGCCTTCTCTGGAGCCCTGCAGGGGTGGTGGTGGTCCAGGCAGGAAAGGGTTTCCTCAGCTC
CACACAGGACAGTGGAGCTGGGCAGTGCTCAGCTCAGACTTGGTGCTCAGGTCAGGCCTCTAGG
ATGGCCAGGCCACATGACGGGGGCCACAAGGGCTCCAGGGCACCACAGACATCTCATGGGGGCT
CTCCAACCCTGGAGAAGGGTGGGCAATTTGGCCCATTGGGTCGGGTCATGGAGCTTCAAGGATT
GCACCAAACCCATATCCCTCAATGGGACCGACCCTATTAAGCAATGGGCCATCCCAGTGGCACC
TCTTTGAGCCATGTGGTGGGTGGCGCTGTTTCTCTGAGGTTGGGTCAAGACTAGCTCCGGCCAG
TGCCTGATGGTGGTGCAGTCCAGTCCTGGGACTCGTGGGCTTTGGGCCAAGGGTGCAGCCCGTG
TCCTACTGGGGATCACCCCTTCCTTATGAGGGAGTGGTGGGAAGGGTATCCTCCTGAGGTAGGA
AGGCCCGCTGTGGAAGGAACTTTCCTGGAAATTTGCAGTCCAGACTGCTTCTGAGCCATGAGGT
CATCTCCGGGGTGGCGGGGTCTTGGGCCCAAGGAAAGGAGAAGACACCATATGTCTCCCGATTG
GTTGAGTATGGCTCCACTCTGTGTGTGGGAGGTGTTGTGTGGGGCATGGATTGGGACCCACCAG
AGAAAGCTCGTGGCACTGGAACCCCAAGCTGCAATTTCCAGTGCACCTTGGGAAGGGGACTGGG
TGCTCACCCTCCATGCACAGGGCAGGCTGTGCAGCTGCCAAGTTGGCAGTGGTGCGTGGCATCC
GGGCCAGCGTCCTTGGTTCGCTGTGAGCTGGTTGGTGTCCTTGACGCCTGCCCAGGGGTCCCAG
TGCTGCTTGGGGTCATGAGGGGAGCCAGCGTGGGCAAGGGAGGCCTGCAGTAGGGCACAGCCCG
GGGGTCTTGCCTGAGCCCAGGATCCTGCCGGCCCTGTCCTTCCATGGTTCGGTGGCTGGGAGGG
CAAAGGGTCCCCCACGTCTGAGACACATCAGTGITTCACATGGATCTCATCACATGGGTCCATA
GAGAGAGAGCAAGCGTGAGCTGACACCCCCCACCCCCTTCCCTGTATAGAGGTCTGAGCCCCTG
GTTGGACACATGTCTTCCTCCTGTCCCCCCAGATGGTGTCTGTGGAGGAAGACTTGCCATTGGG
TTGCTGGCCCCAGGCCAGTGGTTGTCATCCAGGTGCCCAGCCTTGGTGCACTGAAGCTCAGGCC
CTTCCTGGTGCTCTGGTCTCCTGCACTGAGCTGTGGTGAGCACATCCGGGTCCCACTGGATGGG
TGTGTGTGGTGGGGGGTGCCCTGGGTTGGGTCGATGATGAGAACCTTATATTGTCTGAAGAGAG
GTGATGACTTAAAAATCATGCTCAATAGGATTACGCTGAGGCCCAGCCTAGGTGAGAATTTTGG
AAGAGGATGCTGGGAATTACAAGTTCCCTGTGAAGTCCACTGTATTTTGAGCTAGAGACCTGGA
GGCTCTGAGGGCATCTGGAGGGTGCCCAACCCTGTCTCTGCACTCCTCCATGAGGCAGCCCAGG
CTCACTCTGTGGGCTTGGTGTCGGCGCTCCTGTTACCCAAGGGTGGGGTACTTTGGCATCTCCT
CCCTGAGCCCCCATTCTCCCCTGTGTCTTTCAGTGACCTCTTTTGCCCGGCAGACCCCCTGGCA
TTGACCAGCATAGGTGAGTGGATCCTGCTGGGGTCATGGGTCATGAGCCAGGCTGTGTGGGGTT
GCCGAGCATCAGCCTTTGGCTGCAAGGGTTTCCTCAGGAACTGACCCGAATGCCCACCTGGGAT
GGGTTCTCCAGGAGGGCAGCTTCCTTAGGAATCGGACCCAAGGAGGATACCTTCCATGAAGCCC
TGCAGGGGTTGTGGTGGTCCAGGCAGTAAAGGGTTTCCTCAGCCCCATGCAGGGCAGCACCCAG
CTGAAACTCGGTGCTTAAGTCAGGCCTCTAGGATGGCCCGTCCACATGAGGGGAACCACAAGGG
CACTGGGGCGCCAAGGGGGTCTTATGGGGGCTCTCCGACCTTGGAGAGGTATGTGTGATTCGGC
CCATGAGGTCAGGTTTTGGATTTTCAAGGATCTCTCCAAACCCATGGCCCTGAATGGGACCGAC
CCTATTAAGCTATGGGCCATCCCAGTGGTGCCTCTTTGAGCCAAGTGTGGGGTGGCACTGTTTC
CATGAGGTTGGGTCAAGACTAGCTCTGGTGACCCCATGATGGTGCTGCAGTCTGGCCCTGGGAC
TCGTGGGCTTTGGGCCAAGGGTATAGCCCATGTCCCACCAGGGGCCACACCTTCCTTGTGGGCA
AGTGGCGGACAGGGTGTCCTCCTGAGGTGAGAAGGCCTGCTGTGGAAGGACCTGTCCAGGCTGC
TTCCAAGCTATGGGGTCACCTACAGGGTGGTGGGGTCAAGGAAAGGAGAAGACACCATGTGTCT
CCCGATTGGCTGAGTGTGGCCCCACTCTGTGTGTGGGAGGTGTTGTGTGAGGCATGGATGGGGA
CATATCAGAGAACACACGAGGCCCTGGGTCCCCAGGCTGCAATTTCCGGTGTGGTGTGGAGCAG
GGCCTGGGTGCTTGCCCTCCACACACAGGGGCAGGCTGTGCTACTGCCAGATTGGCAGCAGTAC
GTGGCATCCAGGCCAGCCTCCTTGGTTGGCCGTGAGACGGTAGGTGGCCCTGGCTGACACCCAG
GGGCCAGTACAGCCCTGGGGTCATGTGAGGCAACTGGAGTGGGTAGTGGAGGTGCTCAAGGTGC
CTGCAGTAGGGCATAGCCAGGGCGTCTTGCCTGAGCCCAGTGACCTACCTGCCCTGTCCTTCCA
GGGTTCAGTGGTGCAGAGGGCCAAAGTTCCCCTGTGTCCATGACACATCAGTGTTTCACATGGA
TCCCATCACATGGGTCCATGGTGGGAGAGCAATCGTGAGCTAGCATCCCCCACCCCCTTCCTGC
ATGGAGGCGTGAGGTTCCATTTGGACACATGTCCACCTCCTGTCCCCCAGATAGTGAGACTGGA
AGAAAACTTTCATTGGGCCCGCTGTCCCGGGGTGAGTGGTCATCAGTCAGGTGCCCAGCCCTTG
GTGTGGTGAAGCTCAGGCTCTTCCTGGCGCCCTGGTCTCCTGCACGGAGCTGCGGTGAGCACAT
CCGGGTCCCGATGGATGCATGCGTGGGGATGGAGGGCGTGCCCTGAGTTGGGTCAATGATGAGA
ACCTTATATTGTCCTGAAGAGAGGTGATGACTTAAAAATCATGCTCAATAGGATTACGCTGAGG
CCCAGCTTAGGTGAGAATTCTGGAAGAGGATGCTGAGATCCGAAGTCCCCTGGAAGGGCCACTG
TATTTTGGGCTGGAGTCCTTGAGGCCCTGAAGGACACCTTCGGGGTGCCCAACCCAGTCTCTGC
ACTCCTCTGTGTGGCAGCCAAGGCTCCTTGTGCAGGCTTGGTGTTGGCATTCTGGTTCCCTGGG
GGTGGAGCACATCGACGGCTCCTCCCTGAGCCCCCGTTCTTCTCTGTGTCTTTCAGTGAGCTCT
TCTACCCAGGCGGGCCCCTGCCCTTGAGCGGCATAGGTGAGTGGATCTTGCTGGGGTCACGGGC
CAAGAGCCAGGCCACGGGGGGTCGCTGAGGGTCACCTTTCAGGCCACGAGGGTTTCCTTAGGGA
GCTGACCTGAATCCCCACCAGAGATGGATGGTTTCTCCGGAAGGGCAGCTTCCTTGTTAATTGA
TCCCAAGAAGGACGCCTTCCCTGAAACCCTGCAGGGGTAATGGAGGCCCAGGCAGCAGAGAGTT
CCTTCAACCCAATACAGTGCAGCAGCTTGTTGCCTTGATGCTCCCCCTCCCTGGAGAAAGGTGG
GCATAGGGCCCGTGGAGTTGAGTTGCAGAGATTTAAGGATCGCACAGGACTTATTGACCTAAAT
GGGACTGACCCTGTTGAGCAACGTGCACTTGGTGCATGGGTCAGGTGTGGAGCACTGCCCAGGC
CACATGAGGGGTCCACTGGGTCTCTGGGGGCCACGGGTGTTATGCAGGAGCTCTCCGTCCCCAA
GGCTTGCAGGGGGGTAGGATGTCGGTGATCGAGCTTATGCAGTCAGGCCGTGAAGATTCAAGGA
TTGGGCCCAACCCATGGCCCTGATGGGACCGACCCTGTTGAGCAATGGGCTATCCCACTGGTGC
CTCCTTGAGCCTGGTGGGGGCAGTGCGGCCTCAGTGGGCTTGGATCAAGGCTGAGCTCTGGCCA
GCACCTGGTGTTTGGCCCTGGGAATCGTGGGCTTTGGGCCAAGGGTTCAGCCTGTGTCCCACTG
GAGTTCATCCCTACCGGAAGTGGAGTGGATGGAAGGGTGGCATCCTGTGGTGGAAGACCCACCG
TTAAGGGTCCTGTCCTGGCAATTTGATGCCCAGGCTACTTCTGAGCCATGGAGTCACCTCCAGG
GTAGCAGGGTCTTGAGCCAAAGGAAAGTTGAGGACATCATGTGTCCTCTGATTGGCCGAGCGTG
GCCCAGCTCTGTGTTTAGGAGGTGTGCGGTGCATGAATGGGGACCCACCAGAGAAGGCATGTGG
CCCTGGGGCCCCAGGATGTGATTTCTGGTGTGTGCTTATAAAGGGTGCCTGGATGGGTTCATTT
AATGCCCAGGGGCAGGCCATGTGACTGCCAGGTCTGCAGTGGTACGTGGCATCCAGGCCAGCCT
CCTTGATTTGCTGTGAGCTATTTGGTGCCCATGGCCCTGCCCAGTGGTCCTGATGCAGCCCAGA
TTCGAGCAGGGGATTTGGCGTGGGCAGTGGAGGTGCTCCAGGGTCCTGCACTAGGGTGCAGCCT
GGGCGTTTTGCTCGAACCCTGTGACCCTCCTGCCCCGTCCTTCCAGGGTTCAGTGGCCAAGGGT
TCCTCGCGTAAATGACCATCACTGTTTTACATGGATCTCATCTCATGAGTCCATGGAGGGAGAG
CATGTGTGAGCCAGCACCCCCTGCCCACTTTCCTGCATGGAGGCATGAGGCCTTGTTTGGACAC
CTGTCCCCCACCCTTCCAGATGGTGACCCCAGAGGAAGACTTGCGTTGGGCCCACTGGCTCTGG
GCTCAGCCCCTGGTGAGCTGAAGCTCAGGCCCTTCCTGGCACCCTGGTCTGCTGCACTGAGCTG
CGGTGAGCCTATCTGGGTCCCACTGGATGCATGGGTTGCGGGGCAGTGGAGGGTGATGCCCTGG
GTTGGGTCGATGATGAGAACCTTATATTGTCCTGAAGAGAGGTGATGACTTAAAAATCATGCTC
AATAGGATTACGCTGAGGCCCAGCCTAGGTGAGAATTTTGGAAGAAGGTGCTGGGATCCGGAGT
TCCCTGGAATGACCAATGTATTTTGGGCTGGAGACCTTGAGGCCCTGAACAGCAGCATCTCGTG
GGGGCCCAACCCCTTCTCCGTGCTCCTCTGTGAGGCAGCCCAGGCTCCCTGTGTGGGCTTGGTG
TTAGTGCTCCGGTTCTGTGAACCCCCATTCTCCCCTTGTGTCTTTCAGTGAGCTCTTCCACCCA
GGTGCAACCCCCTGGCATTGACCAGCATAGGTGAGTGGTCCCGCTGGGGTCATGGGTCATCAGT
CATGCAGTGTGGGGTCACCAAGAGTCAGCCTTTGAGACAGGAGGGTTTCCACAGGCAGCTGACC
TGAATCCACACCATGGAAGGGTAGGTTGGTCCTCCAAGAGGGCTGTTTTTTTGGGAATCGGACC
CAAGGATGATATCTTTCCTGCAGCCCTGCAGTGATGACGGTGGTCCAGGCAGGAAAAGTTTCCT
TCAATCTCATGCAGGGCAGCTCCCAGCTGAGTCTCAGTGCACTGGTCTGGCCTCGAGGACTGAC
CAGGCCACGTGAGGGGGGCCACAGGGGCTTTGGGATGCCATGGGGTCACACAAGAGCTCCCCAT
CCCTGGAGAAAGGTGGGCATAGAGCCCATGGAGTTGAGCTGCAGATATTTAAGGATCACACAGG
ATTTATAGCCCTGAATGGGACCGACCCTGTTGAGCAATGTGCCATCCATCCTATGGTACCTCTT
TGGGCCTGGATGGGGTGTTGCTGTTTCCCTAGGTTTTGGTCAAGGCTGAGCTCTGGCCAGTGCC
TGGTGGTGCTACAGTCCAGCCACGGACTTGTTAGCTTTAGGCCAAGGGATCAGTCCACGTCCTG
CTGGGGATCTCCCCTTCCCTGTGGCTGGAAGTCACCCCTTCCCCATTGGCAAGTGTGAGGAAGG
GTGGCCTCCTGAGGTGGAAGGCCTGCTGTTGAGGGTCTTGTCCCTGGAATATGCCACCCACACT
GCTTCTGAGCCATAGGGTCACCTCCAGGATAGTGGGTATTGGGCCAAAGGAAAGAAGTCACCAT
GTGTCCCCTGATTAGCCAAGTGTGGCCCTGCTCTGTGTTTAGGAGGTGTGTGGGGCATGGATGG
GGACCCACCAGAGAAAGCATGTGGCCCTGGGACTCCAGGTTGTGATTTCCAGTTCATGCTGGGA
TAGGGCACCTGGATGGGTGCCCACAATGCACCGGGGCAGGCTATGTGACTGCCAGCTTTGCAGC
GGCATCTGGCATCCGGGCCAGCCTCCTTGGTTGGCCATGAACTGGTTGGTGGCCCTGGCCTGTG
CCCAGTAGCTCTGTCGTAATCCTGGGGTCATGCGAGGGAGCCGGCATGGGCAGTGGAGGTGCTC
GTGGGACCTGCGGTAGGACACAGCCAGGTCTTCTTGCCCGAGCCCAGTGACCCACCTGCCCTGT
CCTTCCAGGGTTCAGTGGTTGAGAGTACAGGGTGACTTGTGTCTGTGACAAGCCAGTGTTCTGC
ATGGATCCCATCACGTGGATCCATGGACGGCAAGCATCCCTGAGCCGCCACACCCCACCTTCTG
CCTCCATGGAGGGGTGAGGTTGTGTGTGGACGCCTGTCCTACTCCTGTTTCCCCAGGTATTGAG
CCTGGAGGAAGACTTGAATTGGTCCCAATGGTCCCAGGCCAGCATCCATCAGCTAGGTGCTGAG
CCCCTGGTGCACTGAAGATTGGGCACTTCCTGGCACCCTGGTGTGCTGCACCGAGCTGTGATGA
GCACATCCGGGTCCTGCTGGATGCATGCGCCGGGAAGGACGTGCCCTGAGTTGGGTCGATGATG
AGAACCTTATATTATCCTGAAGAGAGGTGATGACTTAAAAATCATGCTCAATAGGATTACGCTG
AGGCCCAGTCTAGGTGAGAGTTTTGGAAGAGGATGCTGGGATCCTGAGGTCCCCTGGTAGAGCC
AATTGTATTTTGGGCTGGAGACCTGGAAGCCCTGAAGGGCATCTGGGAGGCCCAACCCTGTCTC
TGTGCTCCTCCATGAGGCAGCCCTGGCTCCCCATGTAGGCTTGGTGATGGCCTTCCTGTTCCCT
GGGGGTGGGGCACATGGGTAGCTCCTCCCTGACCCCCATTCTCTCCTGTGTCTTTCAGTGAGCT
CTTCCCCACAGGTGGGCCCCCTGGCATTGACTAGCATCGGTGAGTGGATCCTGCTGGGGTCGTG
GGCCGTGCACCAGGCTGCATGGGGTCACCAAGTATTAGCCTTCAGGACAGGAGGGTTTCCTCAG
GGAGTCGACCTTAATCCTCACCTGTGGAGAGATGGGTTTGTCCTCTAGGAGGGCAGCTTCCTGG
GAAATCAGACCCAAGACGATGCCTTCCCTGAAGCCCTGCAGGGGTGACGGTGGTCCAGTCTGGA
AAGGGTTCCTTCAGTCTCATGCAGGGCAGCTCCCAGCTGACACTTGGTGTGAGAGTCTGTCCTT
GAGGACTGCCCAGGACAAATGAGTGGGACAACAGAGGCTCCCGGATACCACAGGGATCACTCAG
GGGCTCTCCGTCCCTGGAAATGGGTGGGCGATCGGGCCCATGGGGTGGGGCTGCGGAGATTCAA
GGATCGTGCCCTGCTCATGGCCCTGAATGCAAATGACCCTGTTGAGCAATGGGTCACACCAGTA
GCTCCTTTTTGAGCCTGGTGGGTGGCGGCTCAGCTTCAACTGGTTGGGTCAAGGCTGAGCCATG
ACTGGTGCTTTGTGGTGCCGCAGGCCAACCCTGGGACTCATGGGCTTTGGGTCAAGGGTGCAGC
CCGCCTGCTGCTGGGGGTCACCCCTTCTTCGAGGTCAGTGGCGGGAAGGGTCACGTCCCGAGGT
TGGAAAGCCCCCTCATGAGGGACCTGTCCTGGGAATTTGTTGCCCAGGCTGCTTCTGAGCCATG
TGGTCACTTCCGGGTGGCGGGGTCTTGGGCCCAAGGAAAGGAGAGGGCACCATGTGGCTCCTGA
TTGGCTCAGTGTGGCCCAGCTCTGTGTTTGACAGGGGTCTGGGGTGTGGATGAGGACCCACCAG
ACAAAGCACGTGGCCCTGGGCCCAGGCTGTGATTTCTGGTGCGTGCTTGGAGAAGGATCCTGGG
CAGGCATTCTCTCCAAACAGGGGCAGGTTGTGGGACTGCCAGATTGGCAGCAGTGCCTGGCATC
CAGGCCAGCATCCTTGGTTGGCTGTGAGCTGGTTGGTGACCTTGGACCCTGCCCAGGGTTTCCG
TTGTGGTCCAGAGTCATGCAGTGGGGCCAGTGTGAGCAGTGGAGGTGCTCCAGGGGCCTGCTGT
ATGGCACAGCCAGGGCATCTTACCCAAGCCCTCCTGCACTGGTCTCCTGCACTGAGCTGTGGTG
AGCCCATCTGGGTTCCTGTGATGCCTGTGCGGGGAGGGGGTTGCCCTGGGTTGGGTCGATGATG
AGAAACTTATATTGTCTGAAGAGAGGTGATGACTTAAAAATCATGCTCAATAGGATTACGCTGA
GGCCCAGCCTAGGTGAGAATTTTGGAAGAGGATGCTGGGAATTATGAGTTCCCTGGCAGAACCA
CTGTATTTTGGGCTGGAGACCTGGAGGCTCTGAGGGCATCTGGAGGGTGCCCAACCCTGTCTCT
GCGCTCCTCCGTGAGGCAGCCCAGGCTCACTATGTGGGCTTGGTGTTGGCACTCCTGTTACCCG
GGGGTGGGGCATTTTGGCATCTCCTTTCTGAGCCCCCAATCTCCCCTGTGTCTTTCAGTGAGCT
CTTCTGCCCAGCAGCCTCCCTGGCAGTGACCAGCATAGGTGAGTGGATCCTGCTGGGGTAATGG
CCCATGAGCCAGGCTGTGTGGGGTCACCAAGCATCAGCCTTCAGCCATAAGAGTTTCCTCAGGA
GCTGACCCGAATGCCCACCTGGGATGGGTTCTCCAGAAGAGTGGCTTCCTTGGAAATCATACCC
AAGGAAGATACCTTCCCTGAAGCCCTGCAGGGGTTGCAGTGGTCCAGGCAGTAAAGGGTTTTCT
CAACCTCATGCAGGGCAGTGCCCCACTAAGACTCAGTGCTCTGGTCAGGCCTTTAGGATGGCCA
GGCCACATGAGAGGGGCCACAAGGGCTCTGGGGCACCAAGGGGTTCTTGCAGGGGCTCTTCAAC
CTTGGAGAGGTGTGTGTAATTCAGCCCATGAGGTCAGGCTGTGGATTTTCAAGGATTGCACCAA
ACCCATGGCCCTGAATGGGACAAACCCTAATAAGTTATGGGCCATCCCAGTGGTGCCTCTTTGA
GCCAAGTGTGGGGTGGCACTGTTTCCTAAGGGTGAGTCAAGACTAGCTCTGGCTGGCACCTGAT
GGTGGTGCAATCCTGCCCTGGGATTGGTGGGCTTTGGGACATGGGTACAGCCCATGTCCCATTG
GGGTCACCCCTTCATTGTGGACAGGTGGCGGGAAGCATGTCCTCCTGAGGTGGGAAGGCCTGCT
GTGGAAGACCCTGTCTCAGAAATTTGCTGTCTTGGCTGCTTCTGAGCTATGGCGTCACCTCTGT
GGTGGCGGGAGTCTCGGGTGAATGGATCCTGCTGGGGTCATGGGCCATGAGCCAGGCCGGGGTG
GGGTCCCTGAGCGTCACCTTTCGGGTCATGAGGGTTTCCTAAGGGAGCCGACCTGAATCCCCAC
CAGGGATGAATGGTTTCTCTGGGAAGCAGGTTTCCTTGGAAATCAGTCCCAAGAAGGACACCTT
CCCTGAAACCTTGCAGGGGTAACAGAGGTCCAGGCAAGAGAGGGTTCTTTCAACCCAACGCAGT
GCAGATCCTAGCTGAGACTTGGTGCACAGGTGAGTCCTGGAGGACTGCCCAGGCCACATGAGGG
CATCCACAGGGTCTCCGTGGCCAGGAGTCTTATACGGGAGCCCTCTGTCCCCGAGGCTTGCAGA
GGGGTTGGGTGTGGGTGATCTGGCTTACGGAGTCAGGCCGTGAACATTCAAGGGTCGGGAACCA
CCCATGGCCCTGAATGGGACCGACCCTGTTGAGCAATGGCCATCCTGGTGGTGGCACCTTGAGC
CTGGTGGGGGTGGCGCTGCCTCAGTGGGGTTGGGTCAAGGCTGAGCTCTGCCTGTCACCTTGTG
GTTGGCCCTGGGAATCATGGGCTTTGGGCCAAGGGTGCAGCCTGCGTCCCGCTTGAGGTCACTC
CTACCTGAAGTGGAGTGGCCGGAAGGGTGGCATCCTGTGGTGGGAGACCCACGGTCAAGGATCC
TGTGCTGGCAATTTGCTGCGCAGCCTGGTTCTGAGTCATGGGGTCACCTCCAGGGTGACAGGGT
CTTTGTCAAAGGGAAGGAGAGGACATTATGTGGCCCCTGATTGGCCAAGCATGGCCCAGCTCTG
TGTTTGGGAGGTATGCGGTGCCTGAATGGGGACCCACCAGAGAAGGCACATGGCCCTGGGGCCC
CAGGATGGTATTTCTGGTGTGAGCTTATAAAGGGTGCATGGATGGGTGCATTCCATGCCCAGTG
GCGGGTTATGTGACTGCCAGGTCTGCAGTGGCACATGGGGTCCGGGCCAGCCTCCTTGGTTTGC
CATGAGCTGGTTGGTGGCCCAGGCCCTGCCTAGTGGTCTCATTGCAGCCCAGAGTCATGTGGGG
GAGCTGGCGTGGGCAGTCGAGGTGCTCCAGGGTCCTACAGTAGGGTGCAGCCTGGGCGTCTTGC
TGGAATCCCATGACCCACCTGCCCTATCCTTCCAGGGTTGAGTGGCAGGGAGGGCCAAGGGTTC
CTCGTGTGCATGACTGTCACTGTTTTACATGGATCTCATCTCATGAGTCCATGGTGGGAGAGCA
TGTGTGAGCCAGCACACCCTACCCACTTTGCCCTATGGAGGCACAAGGCCCTGTTTGGACACCT
GTCCCCCACCCCTCCAGATGCTGACCCTGGAGGAAGAATTGCACTGGGCCCACTGGCCCCGGGA
AAATGTCCCTCAGCCAGGTGCTTAGCCCCTGGTAATCTGAAGCTCAGGCCCTTCCTGATGCCCT
GGTCTCCTGCACTGAGCTGTGGTGAGCACATCTGGGTCCCAATGGATGCATGCATGCGTTGTGG
GGTGGTGGTGTCCTGGGTTGGGTCGATGATGAGAACCTTATATGTTCTGAAGAGAGGTGATGAC
TTAAAAATCATGCTCAATAGGATTACGCTGAGGCCCAGCCTAGGTGAGAATTTTGGAAGAGGAT
GCTGGGATTTCTAGGTCCCTGTCATGGGCACTGTATTTTGGACTGGAGACCTCCGGGCCCTGAA
CAGCATCTCGGGGGGGACTCTACCCTGTCTCCACATTACATGTGAGGCAGCCCAGGCTCCCTTT
GTGGGCTTGGTGTCTGCACTCTGGTTCCCTGTGGTAGGGCAGCTTGCTGGGTCCTTCCTGAGCC
CCTGTTCTCCCTTTGTGTCTTCTGTCTTTCAGTGAGCTCTTCAGCCCAGGTGGGCTCCCTGACA
CTGATGCGCATAGGTGAGTGGATTCTGCTGGCATCATGGGCCATGAGCCAAGCTGCGTAGGGTC
ACCAAAGGTCACTCTTTAGGCCACGTGGGTTTCCTCAGGGAGCCGACCTGAATCCCCACCAGGT
TGGGACAGTTTCTCCAGGAGGCTGGCTTCCTTGGGAATCAGACCCAAGACAATGCCTTCCCTGA
AGCCTTACAGGGGTAACAGTTGTCCAGACAGGAAAGGGTCCCCTCAGTCCCATTGAAGGCAGCT
CCCAGCTGAGACTCAGTGTGCGAGTCTGGCCTCAAGGATGGCCCAGGCCACATGAAGGAGGCCA
CAGGGGCTCTGGGGTGCCACAGGGGTCACACGAGAGCTCTCTTTCCCTGGAGTGGGGTGCTCGA
ATGAGCCCATGGGGTCAGGCTGCAGAGATTCAAGGATCGCATCCCAACCGTGGCCCTAAATGCG
ACCGACCATGTTGAGTAATGGGCCATCTCAGTGGGGCCTCTTTGAGCCTGGTGGGGGGCGGGGC
TGTTTCCCTGGGTTTGGGTCAAGGCTGAGCTCTGGCCAGCGCCTGGTGATGCTGCAGGCCAGCC
CAGGTTCTTGTGGGCGTTGGTCCATTCGGCAGCCCTGTCCTCCTGGAGGTCACCCCTTGCCTAT
TGGTGATTGGCTGGATCCTGAGGTGGAAAGACCTGCTGTTGATGGTCCTGTCCCAGGAATTTAC
CACCCAGGCTGCTTCTGAGCCATGGGGTCACCTCCAGGGTGGCGGAGTCTTGGGGCTAAGAAAA
AAGAGGACACTATGTAGCCCACGGTTGGCCAAGGGTGGCCTGACTCTGTGTTTGGGACATGTGC
GGGGCATGGATAGGGACCCGCCAGAGAAAGCACGTGGCCCGGGGACTCCAGGCTTCGATTTCAG
GTGTGGCTTGCAAATAGCACCTAGGCAGGTGCCCACCACGCCCTGGGGCAGGCTATGCAACTTC
CATCTCCACAGTGGCGCATGGCTTCTGGGCCATTCTCCTTGGTTGGCCATGAGCTGATTGGTTC
CCCTGGCCTGTGCCCTGTGGTCCCGGAGTGGCTCTGGGATCGTGTGGGGCAGCCAGCATGGACA
GTGGAGATGCTATCAAGGCCTTCAGTAGGGTGCAGCCCAGGTTTCTTGCCTGAACCCAGTGACC
CACCTACCCTGTGTTTTCAGGGTTTGCTGACAGGGAAGGCCAAACGTGCCCCACGTCCATGCTA
GGTCAGTGTTCCTCATGGACCCCATCACATGGGTATCCCTTACCCCTTCCCCACATGGAAGTAT
GAGGCTCTGTTTGGACACATGTCCTCTTCCTGTCCCCCCAGATGGTGAGCCTGGAGGAAGACTT
GCATTGGGCCCAATGGACCCAGGCCAGTGGCCATCAGCCAGGTGCCCACCCTGGTGTGCTGAAG
CGCGGGACCTACCTGTTGTCCTGGTTTCCTACACTGAGCTGAGGTGAGCACATCTGGGTCCCTC
TGGATGCATGCATGGGGCGGGGGGTGCCCTGGTTTGGGTCAATGATGAGAACCTTATATTATCC
TGAAGAGAGGTGATGACTTAAAAATCATGCTCAATAGGATTACGCTGAGGCCCAGCCTAGGTGA
GAATTTGGAAGAGGATGCTGGGATCCCAAAGTCCCAGTAGGGGACTTTATTTATTTATTTTGGG
CTGGAGCCCTGGAGGCCCTGAAGGGCATCTGGGGGGCCCAGCCCTGTCTCTGTGCTCCTCCGTG
AGGCAGCCCAGCCTCCCTGTGTGGGCTTGCTGTTGGTGGTCCAGTTCCTTGGGGCGGGGCACGT
GGACAGCTCCTCCTTGAGCCCCCCTGCTCCACTTGTGTCTTTCAGTGAGCTCTTCAACCCAGGC
GGGGCACCCTGGCATTCACCAGCATAGGTGAGTGGATCCTGCTGCCATCGTGGGCCATGGGCCA
GGCTGGGTAGAGTCACTGAGGGTCAGCCTTCGGGACAGGAGAGTTTCCTCAGGGAGCGAACCTG
AATCCCCACCAGGGAGGGATAGGTTGGTCCTCCAGAAGGGTGGCTTCCTGAGAAATCGGACCCA
AGGGGGACGCCTTCCCTGGAGCCCTGCAGTGATGACGGTGGTCCAGGCAGGAGAGGGTTCCTTC
AGTCTCACACATAGTAGCTCCCAGCTGAGTCTAGGIGTTCGAGTCTGGCCTTGAGGACTGCCCA
GGCCACCTGAGGGGCCAACAGGGACTCTGGGACACCATGGGGGTCACATAGGAGCTCTCCATTC
ATGGAGAAAGATGGATGATAGGGCCCATGGAGTCGAGCTGTGGAGATTTAAGGATTGCGCCCAA
TGTATGGCCCTGAATGGGACAGACCCTGTTGAGTAATGAGCCATTCTATGGGCCTCATCGAGCC
TTGAGCGGGGCGGTGCTGTTTCCCTATGTTTGAGTCAAGGCTGGACTCTGGCCGGTGCCTGGTG
GTGCTGAGGTCTGGCCATGGACTCGTTGGCTTTGGGCCAAGGGGTCAGCCTGCTCCCCCTGGAA
GTCCCCCCTTCCCCGTGGCTGGAAGTCATCCCTTCCCAAGGGGCAAGTGTCGGGAATGGTTGCC
TCCTGAGGTTGCAAGGCCTGCTATCTAGGGTCTTGTCTTTGGAATATGCTGCCCATGCTGCTTC
TGAGTCATGGGGTCACCTCCAGGGTAGCGGATCTTGGGCCCAGGGACAGAAGATGCCGTGTGTC
CCCTGATTAGCTGATTGTAGCCCTGCTCTGTGTTCAGGAGGTGTGTGGGGCATGGATGCAGACC
CACCAGAGAAAACTGTGGCCCTTAGACCCCAGGTTGCAATTTCCATTATGTGCTGGGATAGGGC
ATCTGGGTGGGCACCCACTATGCACAGAGATAGGCTATGTGACTGCCAGGGCTGTAGCGGCATC
TGACATCTAGGCCAGCTTCCTTGGTTTCCTGCGAATGAGTTGGTGGCCCTGGCCTGTGCCTAGT
AGTTCTGGGGTGACCCCGGGTCGTGTGGGGGAGCCAGCAGGGGCCCAGTGACCAGCCTGCCCTG
TCCTTCCAGAGTTCTGTGGCTAAGAGAGCACTGGGTACCCTGTGTCCGTGATGGGCCAGTGTTC
CGCATGCAAACGATCACATGGGTCCATGGAGGGCAAGCATACTTGAACTGCCACACCCCACCTT
CTGCTCACATACAGGAGTGAGGTTGTGTTTGGACAGCTGTCCTACTCCTGTTCCCCTAGACTGT
GAAATGGGAGGAAGAATTGTGTTGGGCCCAATGGCCCTGGGCCAGTATCCGTCAGCCTGGTGTC
CAGCCCCTGGTGTGCTGAAGCTCATGCCCTTCCTGGTGCCCTAGTTTCCTGCACTGAGCTTGGT
GAGCCCATTTGGGTACTGCTGGATGCATGGGTGGGGAGGGAGGTGCCCTGGGTTGGGTCGATGA
TGAGAACCTTATATTGTCCTGAAGAGAGGTGATGACTTAAAAATCATTCTCAAAAGGATTATGC
TGAGGCCCACCCTAGGTGACAATTTTGGAAGAGGACACTGGAAATCAATGAGGTCCCTGGCAGG
GCCACTATAATTTGGGGTGGAGACCTGGAAGCCCTGCAGGGCACCTCGTGGGGGCCCAACCCTG
TCTCTTCACTCCCCTGTGAGGCAGCCCAAGCTCCCTCTGTAGGCTTGGTGTCGGCACTCTGCTT
TCCTGGAGGCGGGGCATTTTGGCTGCTCCTTCCTGAGCCCCTGTTCTCCCCTGTGTCTTTCAGT
GAGCTCTTTTGCCCAGAAGACTCCCTGGCATTGATCGGCATAGGTGAGTGTCTCCTGCTTGGGT
CATGGGCCATGAGCCAGACCATAGGGTTGCCGAGGGTCAGCATTTGAGCCATGAGGGTTTCCTC
AGGAGGCGACCTGAATCCCCACCTGGGATGGGTTCTCCAGGAGGGCAGCTTCCTTGTGAATCTG
AGCCAAGTAAGATGCCTTCTCTGAAGCCCTGCAGGGGTGCCAGTTGTGCAGGCAAGAAAGGGTT
CCCTCAGCCCCAGGCAGGGCAGCGCCCAGCAGAGACTCGGTGGTTGGGTTAGGCCTCTAGGATG
ACCAGGCCACATGAGGGGGGCCACAAGGGCTCTGGGGCACAACAGGGGCTTCGCAGGGTCTCTG
CGACCCCAAAGAGTGGTGGGCGATTCGGCCCATGGGATCGGGCCCCAGATTCAAAGATCACACC
ACACCCATGGCCCTGAATGGAACCGACCCTGTTAAGCATTGGGGCATCCCAGGGGTGCCACTTT
GAACCTGGTGTGGGGTGCCGCTGTTTGCCTGAGGTTGGGTCGAGACTAGCTCCGGCCAGCACCT
GATGGTGGTGCAGTCCGGCCCTGAGATTAGTGGGCTGTGGGCCCAGGGTGCAGCCTGTGTCCCG
CTGGGGGTCACCTCTTCCTTGTGGGCGAGTGGTGGAAGGGCATCCTCCTGAAGTGGGAAGGCCC
GCTGTGGAAGGAACTGTCCTGGAAATTTGCCATCCAGGCTGCTTCTGAGCCATGGGGTCGCCTC
CAGAGTGGCAGGGTCTTGGGCCCAAGGAAAGGAGAGGACACTGTGTTTCCCTCAATTAGCTGAG
TGTGGCCCTGCTCTCTGTGTGGGAGGTGTTGTGTGGGGCATGGATAGGGACCCACCAGAGACAA
CACATGGCCCTCGGGCCCCAGGCTGCAATTTCTCAGTGCATGCTTGGAGACGGTCCTGGGCCTC
CTCCATGCCCAGGGTCAGGCTGTGCAACTGTCAGGTTAGCGGCGGCATGTGGTGTCTGGGCCTG
TCTACTTCGTTGTCCGTGAGCTGCTGGTGGCCCTAGCCCTTTCCCAGGGGTTCAGGTGCAGCCC
TGGGGTTGTGTGGGACAGCCAGTGTGGGCAGTGGAGCTGCTCTGGAGGCCTGCTTTAGGGTGCA
GCCTGGGTGTCTTGCCCGAGTGTAGTGACCCGCCTGCCGTGTCCTCACGGGGTTCCCTAGCAGG
AAGGGCCAAGGGTCCCCTGTGTCTGTGACACGTGAGTGTTTCCCATGGGTCCCATCAAATGGGT
CCACGGAGGGAGAGCATGAGTGAACTGGTACCCCCCACCCCCACCCTCTTCCCCACCTGGAGGC
ATGAGCCCCTGGTTGGACATATGTCCTCCTCCTGTACCCCCAGATGGCAGCGCTGGAGGAGGAC
TTGTACTGGGGCCGCTGGCCCCACATCAGTGTCCGTCATCCCGGCGTCCAGCCCCTGGTGTGCT
GAAGCTCAGGCCCTTCCTTGTGCCCTAGTCTCCTGCACTGAGCTTGGTGAGCCCATTCGGATAC
TGCTGGATGCATGGGTCGGGAGGGAGGTGCCCTGGGTTGGGTCGATGATGAGAACCTTATATTG
TCCTGAAGAGAGGTGATGACTTAAAAATCATTCTCAAAAGGATTATGCTGAGGCCCGGCCTAGG
TTAGAATTTTGGAAGAGGATGCTGGGATCCTGAGGTCCCCAGGAGAGCCCCTTTATTTTTGGCT
GGAGACCTGGAGGCCCTGAAGGGTATCTGGGGGGCCCAGCCCTGTCCCTATACTCCTCTGTGAG
GCAGCCCAGGCTCTCTGTGTGGGCTTGGTGTTGGCACTCTGGTTTTCTGGGGGCGGTTCAGGTT
GCTGGTTCCTTCTGAACCATTGTTCTCCCCTTGATTCTTTCAGTGAGCTCTTCTGCTCAGGCGG
GTCCCCTGGCATTGACCAGCATAGGTAAGTGGATCCTGCTGGGTTCATGGGCCATGAGCCAGAC
CACATGGAGTGACTGAGGGTCAGCCTTCAGGACAGGAAGATTTCCTCGGGGAGCCAACCTGAAT
CCCCACCGTGGAGGGATGTGTTAGTCCTCCAGGAGGGTGGCTTCCTAGGGAATCTGACCCCAGG
AGGACACCTTTGCTGAAGCCCTGCAGGGGTGATGGTGGTCCAGGCTGGAAAGTTTTCCTTCAGT
CTCATGTGGGGCAGCTCCCTGCTGACATTCCGTGCCTGCGTCTGACCTTGAGGACTGCCCGGGC
CACATGAGTGGGGCAACAAGGGCTCCAGGGTGCCACGGGGATTATGAGTGGGGCTCTCCATCCC
CGGAGAGAGGTGGGCAATCAGGCCCATGTGGTCAGGCTTGTAGGGTTCGGTGGTGGGGAGAGCC
AAGGTTTCCCTGGGTCCATTACCGTCACTGTTTCACCTGGATCCCATCACATGGGTCCATGGAG
GGAGAGCATGTGTGAACAGGCACCCCTTGCTCCCTTTCCCACATGAGCCCCTGGTTGGACATAT
GTCCTCCTCCTGTCCCCCCAGATGGTGGCCCTGGAGGAGGACTTGTATTGGGGCCACTGGCCCC
ACGTCAGTGTTCGTCAGCCTGGCATCCAGCCCCTGGTGTGCTGAAGCTCAGGCCCTTCCTGTTG
CCCTAGTCTCCTGCACTGAGCTTGGTGAGTCCATTCGGGGACTGCTGGATGCATGGGCGGGTAG
GGAGGTGCCCTGGGTTGGGTCGATGATGAGAACCTTATATTGTCCTGAAGAGAGGTGATGACTT
AAAAATCATTCTCAAAAGGATTATGCTGAGGCCCGGCCTAGGTTAGAATTTTGGAAGAGGATTC
TCGGATCCTGAGATCCCCAGCCAGGCCACTGAATTTTTGGCTGGAGTCCTTGAGGCCCTGAAGG
GTGTCTGAGGGCTGCTGAAACCTGTCTCTGTGCTTGTCCGTGAGGCAACCCACATTCGCTGTGT
GGACTTGGTGTTGGCCTTCCCGTTCTCCGGGGATGGGACATACTGTCAGCTTGTCCCTGAGTAC
CCGTTCTCCCCTGTGTCTTTCAGTGAGCTCTTCTGCCCAGGTGGTCCCCCTGGTCTTGATGGGC
ATAGGTGAGTACATCCTGCTGGTGTCCTGGTCCATGAGCCAGGGTGCGTACGGTTGCCGGAGGT
CACCTTTAGGCCATGTGGGTTTCCTCAGGGAGCTGACCTGAATCCCCACTGGGAGGGATGGTTT
CTCCAGGAGGATGTCTTCCATAGAAACTGGTTCCAAGAAGGATGCCTTCCCTGAAGCTGTGCAG
GGGTGACGATGGTCCAGGCAGGAAAGGGTCCATTCAACCCAATGCAGTACAGCTTCTAGCTGAG
ACTCGGTGCACGGGTGAGGGCTTGAGGACTCCCAGGCAACATGAGGGGTTCCACAGGATCTCCG
GAGGCCACAGGTGTTAAGCAGGAACTCTCCATCCTTGAGGCTTGCTGGGGGTGGCGGGATGGCA
GCAGGGTGTGGGTGATCGAGTTTATGGATTCAGGCCAAGATTCAAGGGTCAGGCCCCACCCATT
GCCCTGATGGGACCAACCCTGTTGTGCAATGGGCTGTCCCACTGGTGCCTCCTTGAGCCTGGTG
TGGGCAGCGTGGCTTCAATGGGGTTGGGTCAAGGCTGAGCTCTGGCCCGCACCTGGTGGTTGGC
CCTGGGAATTGTGGACTTTGGGCCAAGGGTGCAGCCCGCCTGCCTCTGGAGGTCATCCTACCCA
GAGGTGGAGTGGCCGGAAGGGTGGCAGGCATCCTGTGGTGAAAGGCCCACTGTTGAGGGTCCTG
TCCCACCAATTTGCTGTTCAGGCTGCTTCTAAGCCATGGGTTCACCTCCAGAGAGGCGGGATCT
TGAGCCCAGGGAAAGGAGAGGGCATCATGTGTCCCCTGATTGGCTGAGAATGGGGAGAGTGTTT
GGGTGGTATGTGGTGCATGAATGGGGACCCACCAGAGAAGGCGCGTGGCCCTGGGCCCCAGGAT
GCAATTTCCGGCGTGCTTATAGAGGGCGTCTGAGTGGACACTCTCCATGCCCAGGCGCAGGCTA
TGTGACTGCCAGGTCTGCAGCAGTTTGTGGCATCTGGGCTAGCCTCCTTGATTGCTTATGAGCT
GGTTGATAGCCCTGGCCTGTGCCGAGGGGTCCTGGTGCGGCCCTGGGGTTGTTCAGGGGAAGCC
GGCATGGGCAGTGGAGGTGCTCTTGGGGCCTGCAGTAGGGCACAGCCCAGGTGTTCTTGCTTGA
GCGCAGTGAACCACCTGCCCTTTCCTTCCAGGGTTCGGTAGCAGGGAGGGCCAAGGGTCCCCCA
CGTCCATGACAGGTCAGCGTTCCTCCTGGATCCCATCACATGGGTACATGCAGGGAGAGCATGT
GTGAGCCGGCACTCCCCACTCTCTTCCCCGCATTGCAGGGTGAGGCCCTGTTTGGACCCATCTT
CCAGCTATCTCACCAGATGGTGAGCCCAGAGGAAGACCCCGTGGCTCTGGGCCAGTGTGCGTCA
GCCAGGTCCCCAGCCCCCAGTGTGCTGAAGCTCAGGCCCTTCCCAACGCCCTGGTCTCCTGCAC
TGAGGTGTGGTGAGCACAACCAGGTCCCACTGGATGCATGCACAGGGAGGGATGTGCCCTAGGT
TGGGTCGATGATGAGAACCTTATATTGTCCTGAAGAGAGGTGATGACTTAAAAATCATGCTCAA
TAGGATTATGCTGAGGCCCAGTCTAGGTTAAAATTCTGGAAGAGGATGCTGGGATCCCAAGTTC
ACAGGCAGGGCCACTGTATTTTGGACTAGAGAACTCGAGGCCCTAAAGGGCATCTTGAGGGGGT
CCAACCTGTCTCTGTGCTCCTCCATGAGGCAGCCCTGGCTCCCTGTGTGGCCTTGGTGTTGGTG
CTCTGGTTCCCAGGCGTGGCATGTGGGCACCTCCTTCCAGAGCCCCCATTCTCCCCTGTCTCTT
TCAGTGAGCTCTTCCGCCCATTTGGGCCCTCTGACATTGACCGGCATAGGTGAGTGGATCCTCT
TTGGGTCATGGGTCATGAGCGAGGCAAAGTGGGGTCACCATGCATCTGCCTTCGGGCCATGAGA
GTTTCCTAAAAGTGTGGACCTGAATTCCCACCGGGGATGAGTTATCCAGAAGGGCCACTTCCTT
GGGAATCGGACCCAAGAAGGACGCCTGTCCTGAAGCCCAGCAGGGGTGGTGGTGGTCCAGGCAG
GAAAGGGTTCATTCAGCACCAAGCAGGCAGCTCCCATCTGAGACTTGGTGCATCGGTCAGGCCT
TGAGGACTGCCCAGGACACATGAGGTGGCCACAGGGGCTCCGGGGCACTACAGGGGGTCTGCAG
AGAGGGGTAGGCGATTGGGTCCGTGGGGGTCATGCCGCGGAGTTTCAAGGACACCCATGGCCCT
GAATGGGACTGACCCTGTTGAGTAATGGGCCATCCCAATGGCACCTCCTTGAGTCTGGCGGTGG
GTGGTGCTGTTTCCCTGGGGCTAGGTCAAGGCTGAGCTCTGGCTAGTGCCTGTGGTGCTGCAGG
CTGGCCCTAGAACTCATGGGCTTTGGGCCATGGGGACAGCTTGTGTCATGCTTGGGGTCACCCC
TTCCCGGAGGTTGAATGGCGGGAGTGGTGGCTTCCTAAGGTGGGAAGTCCTGCTATTGAGCAAC
CTGTCCTGGGAATTTGCCACCCAGGCTGCTTCTGAGACATGAGGTCACCTCCAGAGTGGCAGCA
TCTTGGGTCCAAGGAAAGAGGAGCATAACATGTGTCCCCAGATTGGTTGAGTGTGGCCTAGCTC
TGTGTTTGGGAGGTGTGCAGGGTGTGGATGGGGATGTATCAAAGAAAGTACATAGCCCTGGAGC
CCCAGGCTGTGATTTCCAGTGTGTGCTTGGAGAGGGGCAGGAGCAGGCTCTCGACTGCCAGGTC
TGCAGCAGCGCGTGGCATCCGGGCAAGCCTTCTGGTTGGCCATGTGCTGGTTGGTGGCCCTGGC
CTGTGCCCAGTGGTCTCAGGGTAGACCTGGCATTGTGCAGGGCAGCCAGCAGGGCAGTGGAGGT
GCTCTCAGGACCTGCAGTAGGGCGCAGCCTGGACTTTTTGTGTGAGACCAGTGACCCACCTGCA
CTGTCCTTCCAGGGTTGGGTGGCTGGGAGGGCCAAGGGTGGTCCATGTTTGTCTCCGTGACACA
TCAGTGCTCTGCATGGATCCTATCACATGGATATCCCCTACCCACCTTCCCCACATGGAAGCAT
GAGGCCTTACTTGGACATGTGTCCTCCTGTCCTAGATGGTGAGCTCGGAGGAAGACTTGCATTG
GGCCCAATGGAGCTGGGCCAGTTTCTGTCAGCCAGGTCCCCAGCTCCTGATGCACTGAAGCTCA
GGCCCTTCCTGGAGCCCTGGTCTCCTGCCCTGAGCTGTGGTGAGCCCATCTGGGTCCCACTGGA
TGCATGCATGGGAGGGGGTTGCCCTCGGTTGGGTCGATGATGAGAACCTTATATTTTCTGAAGA
GAGGTGATGACTTAAAAATCATGCTCAATAGGATTACGCTGAGGCCCAACCTAGGGGAGAATTT
TGGAAGAGGACGCTGGGATCCTGAGGTCCGTGGCAGGGCCACCGTATTTTGGGCTGGAGACCTC
AAGGCCCTGAAGGGCATCTTGGCAGCCCCCAGCCCTGTCTCTGCACTCCTCCGTGAGGCAGCCC
AGGCTCCCTGTGTGGGCTTGGTGTCAGAGTCCCATTCCCCAGGGGCGGGGCAGGTTGGCGGCTC
CTCCCTGAGTCCCCTTTCTCCCTTGTGTCTTTGAGTGACTTCTTCTGCCCAGGTGGGCCCCCTG
GCGTTGACCAGCATAGGTGAGTGGAACCTGCTGGTGTCATGGGCCATGAGCCAGGCCATGTGGG
GACTCCAGGTCAGCCTTCAGGACAGGAGAGTTTCCTCAGGGAGCAGGGGAATCCCCACTGGGGA
GGGATGGGTTCTCCCAGAGGGCCACTTTCTTTTGAATCGGACCCATGGAGGACATCTTCTCTGA
AGACCTGCAGAAGTGATGATGTTTCAAGCAGCAAAGGGTTTTTTCAGCCCCTGCAGGGCAGGCA
GCTCCCAGATGAGATTCTGTGCATGGGTCAGGCCTCAAGAACTGCCCAGGCCACATGCGGGGTG
CCACACAGGCTGTGAGGCACCACAGGGGTCATGCAGGAGCTCTCGGTCTTCAGGCTTGTTGTGG
GGGTGGTGACAGAGTGATCAGGCCCACAGGGTCAGGCTGCAGTGTTTCAAGGATCGCGCCCACC
TGTGGCCCTGAATGGGACCGATGCTGTTGAGCAATGAGCCATCCCAGTGGCTCCACATTGAGCC
TGGTGGGGGCGCCACTGTTTCAGTGGGGATGGGTGAAGCTTGAGCTCTGGCCAGCACCTGATGG
TGTTGCAGGCTGGCCCAGGGACTTGTGGGCTTTGGGCCAGGTCGGCAGCCTGAGTTCTGCTGGG
GGTCACATCTTTCCTGAGTTTCAGTGTCAGGATGCATGGCCTCCTTAGGTGGGAAGGCCTGCTG
TGGAGGGTCCTGTCCTGGGAATTTGCTGCCCAGGCTGCTTCTGAGCCATGGCGTCACCTCCAGG
TGGTGGGGTCTTGGGCCCAAGGAGCAGAGAAGACACCATGTGGCCCCTGATTGGTCGAGTATGG
CCTGGCTCTGAGTTTGGGAGGTGTGCAGGGCATGGATGGGGACCCACCAGAGAAAGCACGGGCC
AATGAAGCCCCAGGCTGCGATTTCTGATGCGCACTTGGAGAGGGCGCCTGGTTGGGCGCCCACC
ATGCCCAAGGGTAGGCTATGCAACTACCAGGTCTGCGGCAGTGCCTGGCATCAGGACCAGCCTC
CTTTGTTGGCTGTGAGCTGGTTGGTGGCCCTAGCCCGTGTCCAGTGATCTCTGTGCAGCCCTGG
GGTCATGTGGAGCAGCCAGTGTAGGCAGGGGAGGTTATCTTGAGGTGTGCAGTAGGCACATCCC
AGGCATCTTGCCCGAGCCCAGTGACCTCCCTGCCCTGTCCTTCCAGGGTTCGGTGGCGGGGAGG
CCCAAGGGTACCCTGTGTCCATGACAGGTCAGTATTTCATGTGAATCCCATCACATGGGCCCTC
CTCCTGTCCCCCCAGATGGTGAGCCTGGAGGAAGACTTGCACTGGGCCTTCTGTGCGCCGGCCA
GAGGCCATCAACCAGGTGCCCAGGTCCTTTTGTACTGAAGCTTGGGCCCTTCCTGGCACCCTGG
TCTCCTTCATGGAGCTGGTGAGCCCATCCGGGTTCTTCTGGATGTATGCATGGGGAGCGGGGTG
CACTGGGTTGGGTCAATGATGAGAACCTTATATTGTCCTGAAGAGAGGTGATGACTTAAAAATC
ATGCTCAATAGGATTACGCTGAGTCCCAGCCTAGGTGACAATTTTGGAAGTGGACACTGAGAAT
CACAAGGACCCTGGCAGGGCCACTGTATTTTGGGCTGGAGACCTGGAGGCCCTCAAGGGCCTCT
CACAGGGACCCAACCCTGTCTCTGCACTCCTCCGTGAAGCACAGCAGGCTCCCTGTGCGGACTT
GGTGTTGGCACTGTTTCCCAGGGGCAGGGCACATTGGTGGCTTTTCCCTGAGTCCCCATTCTTG
CCTTGTGTCTTTCAGTGAGCTCTTCAGCTCAGAAGGGCCCCTTCACCTTGATTAGCCTAGGTGA
GTGCATCCTGCTGGTGTCATGGGCCATGAGTCAGGCCGCGTGGGGTCACCAAGGGTCAGTCTTT
AGGACAGGAGGGTTTCCTCAGGGAGCCAACCTGTATTTCCACCGGGGAGGGATGGTTTCTACAG
GAGGGCGGCTTCCTTGGGAACTGGACCCAAGATGTTGCCTTCCTGAAGCCCTGCAGGGATGACA
GTGGTCCAGGCAGCAAAGACTTCCTTCAGCCCCGTGCAGGAGAGCTCCCAGCTGAGACTCAGTG
CATGTGTCAGGCCTCGAGGATTGCCGAGGCCACATGATGGGGGCCACAGGGCCTCCGGGGCACC
ACGGGGGTCATGCAAGAGTTCTCTTTCCCTGGAGAGGGGTGCTTGAATGAGCCTATGGGATTGG
GCCGTGGAGATTCAAGGATTGTACCCAACTATGTCCCAAAATGGGACTGACCCTGTTGAGCTAT
GGACCATCCCATGGTGCCTCTTTGAGCTTGGTGGGGGGCAGTGCTGTTTCCCTGGGTTTGGGTC
AAGGCTGAGCTCTGGCCAGTGCCTGGTGAGGCTGTGGGCCGGACCTGGGACTCGTGAGCTTTAG
GCCAAGTCGACAGCTTGTGTCCTGCTGGAGGTCACCCCTTCCCCATGGGTGAGTGGCTGGAAGA
GTGGAATGTGCGGTGGGAAGGCCCGCTGTTGATGGTCCTATCCTGAGAATTTGCTGCCCAGACT
CTCTGTGCCATGGGGTCATGTCCAGGGTGGCAGGGTCTTGGGCCTAGGGAAAGGAAAGGACACC
ATGTGGCCCAGGATTGGCCAAGGGTGGCCCGGATCTGTGTTTGGGACATGTGTGGGGCATGGAT
GGGGACCCACCAGAGAAAGCACATGGCCCTGGTACTCCAGGCTGTGATTTCAGGTGTGGCTTGG
AGAGAGTGCCTGGGCAGGTGCCCACCATGCCCAGCGGCAGGCTATGCAACTTCCATGTCCACGG
TGGCACATGGCTTCTGGGCCAGGCTCCTCGGTTGGCCATGAGCATGTCGGTGACCCTGTCCCAT
GCTCAGTGGTCCTGGGGCAGCCCTGGGATCGTGTGAAGGAGAAGTCAGAGGGGGCACTGGATGT
GCCCTCAAGGTCTGCAATAGCATGAAGACCAGGGGTCTTGCCCAAGCCCAGTGATCCACCTGCC
CTGTTTTTTCAGGGTTTGGTGATGGGGAGGGCCGAGAGTGCCCTGTATCCGTGACAGGTCAGTG
TTCCACATGGATCCCATCACATGGGTATCCCTTACCCCATTCCCCACCTGGAGGTATGAGGCCC
TGTTTGGACACATGTCCTCCTCCTGTCCATCCAGATGGTGAGCCTGAAGGAAGACTTGCATCAG
GACCAATGGACCCTGGCAAGTGTCCGTCAGCCAGGTGCTCAGCCCCTGGTGCACTGAAGCTCAG
GCCCTTCCTGGCCCCTTGATCTCCTGCACTGAGCTGTGGTGAGCACATCCGGGTCCCGCTGGAT
GTATGTGTGGGCAGGGGGGGTGCCCTGGGTTGGGTCAATGATGAGAACCCTATATTGTGTTGAA
GAGAGGTGATGACTTAAAATTACCATGCTCAATGATTACGCTGAGGCCCAACCTAGGTGAGAAA
TTTGGAGGAGGATGCTGGGATCCCGAGATTTCCGGCAGGGCCACTGTATTTTGGGCTGGAGCCC
TGGAGGCCCTGAAAGGTATCTGGAGGAGGCCCAACTCTGTCTCTGCACTCCTCTGTGAGGCAGC
CCAGGCTCCCTGTGCGGGCTTGGTGTTGGCCTTTCTGTTCCCCAGTGAAGTGGCACGTGGGTGG
CTCCTCCCTGAGCCCCAGTTCTCCCCTTGTGTCTTTCAGTGAGCTCTTCTGCCAAGGGGGGGCC
TCCTGGCATTGACCAACATAGGTGAGTGGATCCTGCTGGCATCATGGGCATGGGTTCACTGAGG
GTCAGACTTCAGGCCATGAGGGTTTCCTCAGGGAGCTGACCCCAATTCCCATCAGGGAGGGATG
GTTTCTCCAGGAGGGCGGCTTCCTTGGGAATCTTACCTGAGGAGGATGCCTTCCCTGAAGATCT
GCAGGAATGACAATGGTCCAGATAGCAAAGGGTTCCTTCAGCCCCATGCAGGACATCGCCCAGT
TGAGACTTGGTGTGCAGGTCAGGCCTTGAGGACTGCCCAGGCCACATGAGGCGGGCCAGAAGGG
CTCCAGGGTGCCACGGGTTTCACTTGGGAGCTCTCGGTCCCCGGGCATGTTCCTGGGGGGGGTG
GTGATGGGGTCGGGCCATGGAGGTTCAAGGATCTCATCCCACCTGTGACCCTGAATGGGGCTGA
TCCTGTTGAACAATGGGCCATCCCAGTGGCACGTATTGAGCCTGGTCAGGGGTGGTGCAGTTTC
AATGGGGTTGGGTCAAGGTTGAGATCTGGCCAGCTCCTGGTGGTGCTTCAGGCCAGTCCTGTGA
CCCATGGGCTTTGGGCTAAGGGGGCATCCCATGTCCCATGGGGGTCACCTGTTCTCCATGTTTG
AGTGGCAGTAACAGTGGCATTCTGAGGTGGGAAGGTCCACTGTCTAGGGTGTTGTACCGGGAAT
TTGCCACCCCGCCTTCTTAGCCAAGGGGTCACCTCCAGGGTCTCGGGATCTTGGGCCCAAGGAA
ACGAGAAGATACCATGTGGCCCTTGATTGGCCGAGTGTGGCCCAGCTCTGTGTTTGAGAGGTGT
GGGATATGAATGGGGTCCCACCAGTGAAAGCTCATGGCCCAGGTCCCCAGGCTGTGATTTTTAG
TGCACACTTGGGGAGGGCTCCTCAGTGGGTGCCCACCACGCCCAGAGGCAGGCTATGTGACTGC
CAGGTCTGCAACGGTGCATAGCTTCCAGGCTACCCTCCTTGGTTGGCTGGGAGCGGGTTGGTGG
CCATGGCCCATGCCCAGTGGTCACAGGGCAACCCTGGGATCATGCAGGGGAGCTGGTGAGGGCA
GTGGAGGTGCTCTCCGGAACGGAAGTATGGCTCAGCCCAGGTATCTGGCCCAAACCCAATGACC
AGCCTGCCTTGTTTCTCAGGGTTTGGTGATGGGGAGGGCCAAGGGTGCCCTGCATCTGTGATGG
GTCTGTTCCACATAGAACCCATTACAGGGGATCCCTTCTCCACATGGAGATGTGAGGCCCTATT
TGGACACATATCCTCCTCCTGTCCCCCCAGATGGTGAGCCTGGAGGAAGATTTGCATTGGGCCC
AATGGCCCTGGGCCAGTGTCTGTCAGCCAGTTTCCCAGCCCCTGGTGCACTGAAGCTCAGGCCT
TTCCTGGCATCCTGGCCTCCTGCACTGAGCTGTGGTGAGTATATCATGGTCCTGCTGGATGCAT
GTGCAGGGAGGGAAGGTGCCTTGGGTTCAGTCAGTGTCGAGAACCTTATATTGTTCTGAAGAGA
GGTGGTGACTTAAAAATCATGCTCAATAGGATTACGCTGAGGCCCAGCCTAGTTGAGAATTTTG
GAAGGGGACAGTAGGATCCCGAAGTCCCTGGCAGGGCCCTTATATTTTAGGCTGGAGACTTCAA
GGCCCTGAAGGGCACCTGGAGGTGGCTCAACCTGTCTCTGTGCTCCTCCATGAGGCAGTCCAGG
CTCCCTCTGTGGGCTTTCTGTTGGCACTCTGGTTCCCTGGGCCAGGGAACGTTGGTGGCTCCTC
CCTGAGCCCCTGTTCTCCCCTTGTGTCTTTCAGTGAGTGCTTCTGCCCAGGCAGGCCACTTGGC
ATTGACCGGTATAGGTGAGTGGATCCTGCTGGGGTCATGGGTCATGAGCAAGGCCACATGGGTC
GCCGAGGGTCAGCCTTACAGCCATAAGGGTTTTCTCAGGGAGCCCACCTGAATTCCCACCGGGG
AGGCATAGGTTCTCCAGGAGGGCGGCGTTTTGGGGAATCAGACCCAAGGGGGACACCTTCCCTG
AAGCCCTGCAAGGGTGATGGTGGTCCAGGCAGGAAAGGGTTTTTTTCAGCCCCACACAAGGCAG
CTCCTAGCTGAGAATCGGTGCTTGGGTCAGACCTCAAGGATTGCCCAGGCCACATGAGGGCGAC
TGAGAGCCTCCGGGGCACCACAGGGGTCACGTGGGAGCTCTCGGTGCCTGGCGTGTGATGCACA
AATGGACCTCTTGGGTTTGTTCATGGAGATTCAAGGATCGCACCCCACCATGCCCCTGAATGGG
ATGGTCCCTCTTGAGAAATGGGCCATCCCAGTGGGTCTCCTGGAGCCTGGTTGGGGGGTGGCAT
TGTTTCCCTGGGGTTGGGTGAAGGCAGAGTTCTGGCCGGCACCTGGTGGTGCTGCAGGTCAGCA
CTGGGACCCGTGGGCGTTGGGCCAAAGGAGAGCCCGAATCCCACTGGGGGTCACCCTTTTTCCA
ACATCAAATGTCTGGAAGGGTGGCCTCCTGAGGTGGGAAGTCCTGCTGTTGAGGGACATGTCCT
GGGAATTTGTCACTCAAGCTTCTTCTGAGCTTTGGTGTCACATCCAGGGTGGCAGGGTCTTGGG
TCCAAGCAAAGCAGAGGGCACCATGTGTCTCCTGATTGGCCAAGTGTTGCTGGCTCTGTGTGGG
AGGTGTGCAGAGTATGGATGGGGACCCACGAGAGAAAGCACGTAGCACTGCAACTCCAGGCTAT
GATTTCTGTTGCATGCTTGGAGAGGGTGCCTGGACAAGCGTGCCATGCCCAGGGGCAGGCTGGG
CAACTACCAGGTCTTCAGCGGAGTGTGACATCTGGGCCAGCCTCCTTGGTTGGCCGTGACCTGG
TTGGTGGCCCGGCTCTGTGCCTACTGGCCCCAGGGCAGCCCTGGGATCATGCAGGGGAGCCATT
TTGAACAGTGGAGGTGCTCTTGGGGCCTGTAGTATGGCACAGCCTAGGTGTTTTGCTCAAGCCC
AGTGACCCACCTGCCCTGTCCTCTCCGGGATCGGTGGAAGGGAGGGCCAAAGGTGCTCCGTGTA
CTTGATGAGTCAGTATTTCACATTGAACTCATCACATGTGTCCATGGAGGGTGAGCAAGGGTGA
GATGGCACTGCTGACCCCCTTCCCCACGTAGAGGTCAGTTTGGACACTTGTCTTCCTCCTGTCT
CCCCACATGGCGACCCCAGAGGAAAACTTGTGTTGGGCCCGCTGTCACTGGGCCAATGTCAGTC
AGCCAGGTGCCCAGCCCCTGGTGCGCTGAAGCTTGGGCCCTTCTGGCACCCTTGGCTCCTGCAC
TGAGCTGTGGTGAGCCCATCTGGGTCCTGCTGGATGCATGTGCAGGGAGGGGGATACCTGGGTT
AGGTCGATTATGAGAACCTTATATTGTCCTGAAGAGAGGTGATGACTTAAAAATCATGCCCAAT
AGGATTACGCTGAGGCCCAGCCTAGGTGAGAGTTTTGGAAGAGGATGCTGGGATTCTGAGGTCC
CTGACAGAGTAAATGTATTTTGGGCTGGAGACCTTGAGGCCCTGAAGGGCATCTGGTGGGCCCA
CCCCTATCTCTGTGCTCCTCCGTGAGGCAGCCCAGGCTCCCTGTGCTGGCTTGGTATCTGGGCT
CCGGATCCCTGGTTATGGGGCATGTTGGAGTCTCCTTCCTGAGTCCCCGTTCTCCCCTGTGTCT
TTTGGTGAGCTCTTCTGCCCAAGTGGTCCCCGCGGCCTTGAGCGGCATAGGTGAGTGCATCCTG
TTTCGGTCATGGGCCATGAGTGATGCAGCATGGGGTCCTCGACATTCAGCCTTCGGGCCACTAG
GGTTTTCTCAGGCTGCCAACCTACTCCCCACCAGGGAGCAATGGTTTCTCCAGGAGGGCCACTT
CCTTGGGAATCTGACCCAAGGAGCACATCTTTCCTGAAGCCCTGCAGGGGTGACAGTGGTCCAG
GCAGCAAAGGGTTCCTTCAGCCCAATGTAGGGCAGCTCCCAGCTGAAACTCGGTGGACAGCTCA
GGCCTGGAGTACTGTCCAGGGCACATGGGGAGGAGGGGCACAGGGAATGTGGCGCACCCTAGAG
GTTACACTGGAGTTCTCTCTCCCTGGAGAGGGGTGAGTGATTGGGCCCACACAGTCAGGCTGTG
AAGATTCAAGGAATGCTCCACACCCATGGCCTTGAATGGGACCAACCCTGTTGAGCAGTGGGCC
ATCTTATGGTACCTCCTTGAGTCTGGTGAGGGATGGCACTGTTTCAGTGAGGTTGGGTCAAGAC
TGAGCTCTGGCTGGCACCTGGTGCTGCTGCAGGCCCTGGGACTTGGGGGCCTTCAGTCAAGGGG
GCAGCTCTGATCCTGCTGGGGACAAACCTTCCTTGAGGTCCAGTGGTGAAGAGTGTCCTCCTAA
GGTGGGAAGGCCTGCTGTCAAGGGAATTTGCCACCCAGGCTGCTTCTGAGCCTTGGGGTCACCT
CAGGGTGGCAGGGTCTTGTGCCCAAGGAAAGGAGAAGACACCATCTGGTTCCTGATTGGCTGAG
TGTAGCCGGGCTCTGTGTGTCGGAGTTGGGCAGGGCATGGATGGGGACCCACCAGAGAAAGTGT
GGCCCAGGGGCCCCCCAGGCTTGGAGAGGGCACCAGGGCCGGTGCCCACAATGCTCAGGTGCTG
GCTCTGGGAGGGCCAGGTCTGCAGCGGCTTTTGGCATCTGGCCAACCACCTTGGTTGGCCAGGA
GCTGGTTGATGGCCCTGTCCTGTGTCCTGTGCCCTGTGCCCTGTGTCCCTGGTATGGCCCTGGG
GTCATGTGTGGGAGCTGGCCTGAGCAGTGGAGGTGCTCTTGGGGCCTGCAGTAGGGTACAGCCT
AGGCATCTTGCCTGAGCCAGCACTGTCTTTTGCTTTCCCTGTCTTTCCAGGGTTTGGTGGCAGA
GAGGGCAAAGGATGCCCCACATCTGTGTTGGGTCAGTGTTCAGCATGGATCCCATCACATGGGT
ATCCCTTAGCCCCTTCCCTGCATTGATATGTGAGGCCCTGGTTGGACACATGTCCTCCTTCTGT
CCCTCTAGATGGTGAGCCTGGAGGAAGATGTGTGGGACCCGCAGGCCGAGGGCCAGTGTCTCTC
AGCCAGGTGCTCAGCCCCCAGTGGGCTGAAACTCTGACCCTTCCTGGCACCCTGCTCTCCTGCA
CTGTGCTGTGGTGAGCACATCCGGGTTCCACTGGATGTTTGTGCAGGGAGGGGGTTCCCTGGGT
TGGGTCAATGATGAGAACAGGGCCACATGAGGGGGGCCAGAGGGGCTACAGAGTGCCATGGGGT
AACAAGGGAGCTCTTGGTCCCTGGAGTGTGGTGTGTGAATGGGCCCATGGGGTTGGGTAGCAGA
GATTCAAGGATCCTGCTCTACCCATGGCCCTGAATGTGACCATCGCTGTTGAGCAATGGGCCAT
CCCAGTGGGTCTCCTGGGGCCTGGTTGGGGGGCGGAGTIGTTTCCCTGGGATTGGGTCAAGGTT
GACTCTGGCTGGCACCTGGTGGTGTTGCAGGTCAGCCCTGGGACTCATGGGCTTTGGGCCAGGG
ACAGCCCGCATCCCGCTGGGGGTCACCCAAGTGTTGTGAAGGGTGGCCTCCTGAGTTGGGAAGG
CCCGCTATTGAGGCATCTGTACTGGGAATTTGTTGCTCACGCTGCATGGGAGCCATGGTATCAC
CTCCAGGGTGGCAGTCTTGGGACCAAGCAAAGGAGGGGGCAGCATGAGACTCCGGATTGGCTAT
GTGTGACTGGCTATGTGTCTTGGAGGTATGTGGAGTATGGATGGGGACCCACCAGAGAAAACAA
GTGACCCTGAGGCCCCATGCTGTCTTTTCTGGTGCCTGCATGGAGAGGGTGCCTGTACGCATGC
CCACCATGCCCAGGGGCAGGCTGTGCCACTGTCAGGTCTGCAGTAGCATGTGGCATCTGGGCCA
GCCTCCTTGGTTGGCTGTGACCTGGCTGGTGGCCCGGACCTCTGCCCAGTGGTCCCAGGGCAGC
CCTAGGGTCATGCGGAGGAGCAGTTTTGGCCAGTGGAGGTGCCCTTGGGTCCTGCAGTCGTGCA
TACCCTGGGCGTTTTGCCTGAGCCCAGTGACCTACCTGCCTTGTCCTTCCCGGGTTCGGTGGCA
GGGAGAGACACTTGCATAGGTGATGAGTCAGTATTTCGCATGGATCTCATCACATGTGTCCATG
GAGGGGGAGCAAGCGTGAGCTGGCACCCAACACCCCTTTCTCCACATGGAGGGTGGTCACATGT
TCTCCACCTGTTCCCCTAGATGGTGAGACTTCTGTTGGGCTTGCTTTTCCTGGGCCAGTGTCCA
TCAGCCAGGTGCTCAGCCATCGGTGCGCTGAAGCTCCAGCCCTTCCTGGTGCCCTGATCTCCTG
CACTGAGCTGTGATGAGTACATCCGGGTTCCACTGGATGCATGTGCGGGAAGGGGGGTGCCCTG
GGTTGGGTCAGTGATGAGAACCTTATATTGTCCTGAAGAGAGGTGATGACTTAAAAATCATGCT
CAATAGGATTACGCTGAGGCCCAGCCTGGGGGAGAATTTTGGAAAAGGATGCTGGGATCCCAGG
GTCCCCAACAGGGCCACTGTATTTTGGACCTGGGACCTGGTGGCTCTGAAGGGCATCTGGAGGT
GGCCCAACCTGTCTCTGCACTCTAGTTTCCCTGTGTGGGCTTGGTGTCGGTGCTCCAGTTCCCT
GGGGGCAGGGAATGTTGATGGCTCCTTCCTGAGTCCCTGTTCTCGCCTTTGTCTTTCAGTGAGC
TCTTTCACCTAGGAGGGCCCCTTGGCATTGACTGGCATAGGTGAGTGGATCCTGCTGGTGTTAT
GGATCATGAGCCAGACCATATGGGGTCCCCAAGGATCAGCCTTTGGGCCACAAGGGTTTTCTCA
GGGAGCTGACCTGAATTTCCACCTGAGACGGAAAGGTTCTCCTGGAAGGCAGGCTTTTTGGAAT
CGGACCCAAGGAGGACACCTTCTCTGAAGCCCTGAAGGGGTGATGGTGGTCCAGGCAGGAAAGG
GTTCCTTCTGCCTAACATAGGACAGTGCCTAGTTGAGACTCGGTGCTGCAGTAAGGCCTTGAGG
ACTGCCCAGGCTACATGAGGGGGGCCAGAGGGGCTACGGGGCACCACAGGGTTAATGGTGACCT
CTTGGTCCCCAGAGTGTGGTGCGTGAATGGACCCATGGTGTTGGGTTGTGGAGATTCAAGGATA
GTGCCCCACCTATGGCCCTGAATGCAACTGTCCCTGTTTAGCAATGGGCCATCTCAGTGGGTCT
CCTGGAGCCTGGTGGGGGGCAGAGTTGTTTCCTTGTGACTGGGTTAAGGTAGAGCTCTGGCCAG
CACCTGGTGGTGTGGCAGGTCAGCCCTGGGACTCTTGGGCTTTTGACCAAGGGGCAGCCTGTAT
CCCGCTGGGGGTCATCCTATCTCTGAGGTCAAGTGTCGTGAAGGGTGGCCTCCTGAAGTGGGAA
GGCTCGCTGTCGAGGGACCTGTCCTAGGATTTTATTGCTCAGGCTGCTTCTGAGCCATGGTGTC
ACCTCTAGGGTGATGGGGTCTTGGGAACAAGCAAAGGGGAGGGCACCATGTTGCTCCGGATTGG
GCAAGTGTGGCCCAGCTCTGTGTGTGTGAGGTGTGCAGAACATGGATGGTGACCCACCAGAGAA
AGCACATGGCCCTGTAGCTCTAGGTTGTGATTTCCGTGCACACTTGGAGACAGCATCTTGATGT
GTGCCCAACACGCCCAGGGGCAGACTATGCGATAGCCAGGTCTGCAGCGGTGTGTGGCATCTGG
GCCAGCCACCTTTGTTGGCCATGAGCTGGCTCATGGCCCTGACCCATGCCCACTGGTGTCAGGG
GGCCCTGGCGTCGTGTGAGGAGCCATCTGTGGCAGTGGAGGTAGACTCAGGGCCTGCAGTATGG
CGCAACCCATGCATTTTGCCTGAGCTCAGTGACCTACCTGCCCTGTTCCTCCAGGGTTTGGTGG
CAGGGGGAGCCAAGGGTGCCCCACATTGGTGACGAGTTAGTACTTCACATGGATCTCATAACAT
GTGTCCGTGGAGGGCAAGCAAATGTGAGCCCGCACCCACTACTGCCTTCCCCACATGGAGGTCA
GTTTGGACACATATCATCCTGCTGTCCCCCAAATGGTGAGACCGAAGAAGACTTGCCCTGGGCC
CTCTGTTCTTGGGCCAGTGTCAGTCAGCCAGGTGCCCAGCTCTAAGTGCGCTGAAGCTTGGGCC
TTTCCTGGAATGCTCGGCTCCCGCACTGAGCCAGGGTGAGCACATCCGGGTACTGCTGGATGCA
TGTGTGGGGCAGGGGTGCGCCCTGGGTTGGGCTGATGATGAGAACCTTATATTGTCCTGAAAAG
AGGTGATGACTTAACAATCATGCTCAATAGGATTACATTGAAGCCCAGGATAGGTAAGAATTAG
GGAAGATGATGCAGGGATCCCAAGATCTCCAGCTAGACCACACTTATTTTGCACTGGAGAACTT
GAGAACTTGAAGGGCATCTCAGGGGAGCCCAACCCTGTCTCTGAGCTCCTTCGTGAGGCAGCCC
AGGCTCCCCTTGCGGGCTTGGTGTCTGTGCTCCGCTTCTCCAGGGACAGGGCAATTTGGTGTTC
CTTCCTGAATGCTCCTTCTTCCCTGTGTCCTTCAGTGAGCTCTTCTGCCCAGGTGGGGTCCCTA
GCTTTGAGCGGCATAGGTCAGTGCATCCTGTTGGGGTCATGGGCCATGAGTGAGGCAGCATGGG
GTTGGTGAGAGTCAGCCTTCAGGCCACTAGGGTTTTCTCAGGGAGCCAACCGATTACTCACCGG
GAGGGATGGTTCCTCCAGGAGGGTCACTTCTTGGGAATCTGACCCTAGGAGCACACATTTCCTG
AAGCTCGGCAGGGAGATGGTGGTCCAGGCAGGAAAGGGTTCCTTCAACCTAACGTAGGGCAGTT
CCCAGCTGAAACTAGGTGGGCAGGTGAGGCTTGAGGACTGCCCAGTCCACATGAAAGGGGCCAC
AGGGGCTTTGGGGTGCCCTGGGAGTCATGCTGGAGTTCTTCCCCCAAGAGGGATGGGTGATAGG
GTCCACGTGGTCAGGCTGCGGAGATTCAAGGAATGCCACCCACCCATGGCCCTGAATGGGACCA
ACCCTGTTGAGCAATGGGCCATTTTCGGCATGTTTCAATGCAGTTGGGTCAAGGCTGAGCGCTG
GCTGGTGCCTGGTGGTGCTGCAGGCCGGCCCTGGGGCTCATGGGCTTTATGCCAAGGGGGCAAC
TCCCATCCCACTGGGGTAATCCCTTCCTTGAGGTCCAGTGGTGGGAAGGGTGGCCTCCTGAGGT
GGGCAGACCTGCTGTCAAGGGACCTGTCCTGGGAATTTGCCACCTAGGCTGCTTCTGAGCCATG
GGGTCACCTCCAGGATGGCGGGGTCTTCAGCCCAAGGAAAGGAGAGGACACCATGTGGTTTCTG
ATTGGCTGAGTGTGGCCTGGCTGTATGTGTGGGAGGTGTGCAGGGCATGGATGGGGACTCATCA
GAGAAAGAATGTGGCCCTGGAGGCCCCCAGGCTTTGATTTCTGGTGTGTGCTTGGAGTGGGCAC
CAGGGTGGCCCTACCATGCCCAGGGGCAAGGTCTTTGCCTGCCAGGTCTGGAGTGGCTCCTGGC
ATCAGGACCACCCTCCTTGGCTCAACCAACCTTCAGCAGGTTGGCTGCCTTGGCCCGTGCCCCA
TGGTCCCGGTACGACCCTGGGGTTGTTCGGGGGATCTGGCCCAGGCAGTGGAGGTGCTCTTGGG
GCCCACAGTAGGGCACAGCCCAGGCCTCTTGCCCCAGCCCAGTAACCCACTTGCCCTGTCTTTC
CAGGGTTTTGTGGCAGGGAAGGCCAAGGGTGCTCTGCGTCCATGTTGGGTCAGTGTTCTGTAAG
GATTCCATTACAAGGGTATCCCCTACCCACCTTCCCTACATTGATACATGAGGCCCTGGTTGGA
CACATGTCCTCCTCCTGTCCCTACAGATGGTGACCCTGAAGAAAGACTTCAGTTGGGCCTGAAG
GCCCCGGGCCAGTGTCCATCATCCAGGTGCTCAGCTCCCAGTGCACTGAAGCTCAGACCCTTCC
TGGTTCCCTGGTCTCTTGCACTGAGCTCTGGTGAGCACAACTGGGTGCTGCTGGATGCATGCAT
GGGGAGGGGGGTGCCCTGGGTTGGGGTGGTGATGAGAACCTTGTATTCTTCTGAAGAGAGGTGA
TGACTTAAAAACCATGCTCAATAGGATTACACTTAGGCCGAACCTAGGTGAGAATGTTGGAAGA
GGACGTTGGGATCCTATTATCCCTGGCAGAGCCACTGTATTTTGGGCTGGAGACCTGGAGGCCC
TGAAAGGGCATCTGGAGGGGGCCCAACCCTGTCTCTGTGCTCCTGCATGAGGGTCCCCAGGCTC
CCTGTCCAGCCTTGGTGTTGGTGCTCTGGTACCCTGGGGATGGAACAAGTTGGTGGCTCTTTCC
TGAGCCCCCATTCTCCCTTTGTGTCTTTCAGTGAGCTTTTCTGCCCAGGTGGGCACCCTGGCAT
TGATTAGCCTCCTTGGTTGTCCATGACCTGGCTGGTGGCCCGATCCTGTGCCCAGTGGTCCCAG
GGCAGCCCTGGCATCATACGGGGCAGCTGTTTTGGGCAGTGGAGGTGCTCTCCAGGCCTGAAGT
ATGGCACAGCCTGGGCATTTTACCTGAGACCAGTGACCTGCCTGCCTTGTCCTTCCTGGGTTCG
GTGGCAGGGAGGGCCAAGGGTGCCCTGCGTCAGTGATGAGTCAGTATTTTGTGTGGATCTCATC
ACCTGTCCATGGAGGGAAAGCAAGCATGAGCTGGCACCCAACACCCCCTTCTCCGCATGGAGGG
TGGTCACATGTCCTCCACCTGTGCCCCCAGATGGTGAGACTTGAGGAAGACTTCCACTGGGCCT
GCTTTTCCTGAGCCAGTGCCCATCACCCAGGTGCTCAGTCCCTGGTGCACTGAAGCTCCAGCCC
TTCCTGGCGCCCTGGTCTCCTGAACTGAGCTGTGGTGAGCACATCCGGGTTTCACTGGATGTTT
GTGCGGGGAGGGGGTTCTCTGGGTTGGGTCAATGATGAGAACCTTATATTGTCCTGAAGAGAGG
TGATGACTTAAAAATCATGCTCAATAGGATTACGCTGAGGCCCAGCCTAGGGGAGAATTTTGGC
AGAGGATGCCGGGATCCCAAGATTTCCGGGAGGGCCACTTGTATTTTGGGTTGGAGACCTGGAG
GCCCTGAAGGGCGTCTGGAGGTGGCCCAGTGCTTTCTTTGCGCTCCTCTGTGAGGCAGCCCAGG
CTCCCTTTGTGGGCTTGGTGTTTGCGCTCCTCTTCTCCAGTGATGGGGCACGTTGGGGGCTCCT
CCCTGAGTCCTGGTTCTCCCCTTGTGTCTTTCAGTGAGCTCTTCTGCCAAGCGGTTCCCCTGGC
ATTGACCAACATAGGTGAGTGGATCCTGCTGGCATCATGGGCTATGAGCTAGGCCGCATGGGTT
TGCTGAGGGTCAGACTTCAGGCCCCAAGGGTTTCCTCAGGGAACCGACCTCAATTCCCACCATG
TGACTGCCAGGTTTGCAACAGTGCATAGCTTCCGAGCTACCCTCCTTGGTTGGCCGGGAGCTGG
TTGGAGGCTGTGGCCCATGCCTAGTAGTCACAGGGCAGGCAGACTGCCCTGTTTTTTCAGGGTG
TGGTGACGGGGAGGGCCAAGGGTGCCCTGCATCCATGATGGGTCAGGGATTTCAGGAGGGATGG
TTTCTCCAGGAGGGCAGCTTCCTTGGGAATCTTACCCAAGGAGGATGTCTTCCCTGAAGCCCTG
CAGGGGTGACGATGGTCCAGATAGCAAAGGTTTCCTTCAGCCCCATGCAGGACATCACCTAGTT
GAGATATGGTATGTGGGTCAGGCCTTGAAGACTGCCCAGGCCACCTGAGGGGCCCAGAAGGGCT
CTGGGGTGCCACAGGTGTTATGTAGGAGCTCTCAGTCCCCAGACTTGTTTCGTGGGGTTGGTGA
CGGGGTTGGGTTGTGGAGGTTCAAGGATTTCATCCCACCTGTGACCCTGAATGGGGCCAACCCT
GTTGAGCAACAGGCCATCCCAATGGCACGTATTGAGCCTGGTGGGGTGTGGTGCAGTTTCAATA
GGGTTGAGTCAAGGCTGAGATCCATCTGGTGCTTGGTGCTGCTGCTGGCCAGCCCTGGGACCCG
TGGGCTTTGGGCTAAGGGGGTAGCCCACATCCTATTGAGGGTAACCCATTCTCCGAGTTTGAGT
AACGGTTAACAGTGGCATTCTGAGGTGGGAAGGCCCACTGTCTAGGGTCCTATACTGGGAATTT
GCCACCCCGGGTTCTTCTGAGACAAGTGGTCACCTCCAGGGTCATGGGGTCTTGGGCCCAAGGA
AAGGAGAAGATACCATGTGGCCCTTGATTGGCTACATGTGGCCCAGGTCTGTTTGGGAGGTGTG
TGGGATGTGGATGGGGTCCCACCAATGAAAGCACATGGCCTGGGTCCCCAGGCTGTGATTTTCA
GTGCACACTTGGAGAGGGTGCCTCGGCGGGTACCTACCACATCCATGGGCAGGCTATGTGACTA
CCAGGTTTGCAACAGTGCATAGCTTCCAAGCTACCCTCCTTGGTTGGTCGGGAGCTGGTTGGAG
GCTGTGGCCCATGCCTAGTGGTCACAGGGCAGGCAGACGGCCCTGTTTTTTCAGGGTATGGTGA
TGGGGAGGGCCAAGCATCCATGATGAGTCGGTGTTCCACATGGAACCCATTACGTGGGTATCTC
TTACCCCTTCCTCATATGGAGATGTGAGGCCCTATTTGGACACATATCCTCCTCCTGTACCCCC
AGATGGTGAGCCTGGAGGAAGACTTGCGTTGGGCCCAAAGGCCCTAGACCAGTGTCTGTCAGCC
AGGTTCCCAACCCCCGATGCGCTGAAGCTCAAGCCTTTCCTGGCACCCTGGTCTCCTGCACTGA
GCTGTGGTGAGTATATCCTGGTCCTGCTGGATGCATGTGCAGGGAGGTGGGTGCCTTGGGTTGG
GTCAATGATGAGAACCTTATATTGTTCTGAAGAGAGGTGATTATTTAAAAATCATGCTCAATAG
GATTACGCTGAGGCCCAGCCTAGTTGAGAATTTTGGAGGAGGACACTGGGATACCGAAGTCCCC
CGCAGGGACTCTGTATTTTGGGCTGGAGACCTTGAAGCCCTGAAGGGCATCTGGAGGTGGCCCA
ACCTGTCTCTGTGCTCCTCCCTGAGGCAGCCCAGGCTCCTTTTGTGGGCTTGGTGTTGGGGCTC
CAGTTCCCCAGGTCAGGGAATATTGGTAGCTCTTTACTGAGCCCCTGTTCTCCCTTTGTGTCTT
TCAGTGAGCTCTTCTGCCCAGGCGGGCCCCCTGGAATTGATGGGTATAGGTGAGTGCATCCTGC
TGGGGTCATGGGCCATGAGCCAGGCCACGTGGGGTCACCAGGGGTCCCCCTTCTGGCCATGAGG
GTTTTCTCAGGGAACCGACCTGAATTACCACTGGGGAGGCATAGGTTCTCCAGGAGGGCGGCTA
TTTTGGGAATCGGATCCAAGGGGGACACCTTCCCTGAAGCCCTGTGGGTGTGATGGTGGTCCAG
GCAGGAAAGGGTCCCTTCCACCCCACACAGGGCAGCTCATAGCTGAGAGTTGATGCTCAGGTCA
GGCCTCAAGGATTTCCCAGGCCACATGAGGGGGACAGAGGAGCTCTGGGTGCCACGGGGGTCAC
ATAGGAGCTCTCAGTATTCGGTATGTCATGCACAAATGGGCCTCTTGGGTTGGATTGTGGAGAT
TAAAGGATCGCGCACCACCGTGGCCCTGAATAGGACCATCCTTATTGAGCAATGGGCCATCCCA
GTGAGTCTCCTGGAGCCTGGTGGAGGGGAGGGGGTGGCATTGTTTCCCTGGGGATGGGTCAAGG
CAGAGCTCTGGCCAGCACCTTGTGGCTTCTGCAGGTCAGCCTTGGGACTCATGGGCATTGGGCC
AAGGGGGAGCCCGCGTCCCACTGGGGGTCACCCTTTTTCCGACGTCAAGTATCTGGAAGAGTGG
CCTCCTGATGTGGGAAGGCCTGCTGTCGAGGGACGTGTCCCAGGAGTTTGTCGCTCAAGTTTCT
TCTGAGCTTTGGGGTCACATCCAGGGTGGCGGGGTCTTGGGCCCAAGCAAAGCAGAGGGCACCA
AGTGTCTCCTGATTGGCCATGTGGCTGTCTCTGTGTGTGGGAGGTTTACAGAGTATGGATGGGG
ACCCACCAGAGGAAGCACGTGGCCCTGCAGCACCAGGCTGTGATTTCTGTTGCACACTTGGAGA
GGTTGCCTGGACACGTGCCCTTCATGCCCAGTGACAGACTATGCAACTACCAGGTCTGCAGCAG
GGTGTGACATCTGGGCTAGCCTCCTTGGTTGGCCGCCACCTGGCTGGTGGCCCGGATCTGTGCC
CAACAGTCCCAGTGCAGCACTGGGGTCATGCAGGGGAGCTGTTTTGGACAGTGGAGGTGCTCTC
GGGGCCTGCAATATGATGCAGCCTGGGCGTTTTGCTCAAGCCCAGTGACCCACCTGCCCTTTCC
TTTCTGGGTTCAGTGGAAGGGAGGGCCAACAGTGTAGTGCGTACTTAACAAGTCAGTATTTCAC
ATTGAACTCATCACATGTGTCCATGGAGGGCAAGCAAAGGTGAGCTGGCACCCCCGACCCCCTT
CCCCAAACGGAGGTCAGTTTGGGCACACGTCTCCCTCCTGTCTCCCCAGATGGTGACCCCGGAG
GAAAATTTGTGTTGGGCCCACTATCCCTGGGCCAATGTCAGTCAGCTAGGCACCCAGCCCCCAG
TGCGCTGAAGCTCAGGACCTTCCTAGTGCCCTCGGCTCCTGCACTGAGCTCTGGTGAGTCCATC
TGGGTCCTGCTGGATGCATGAGCTGGGGGCGGGAGGGTGCCCTGGGTTGGGTCAGTGATGAGAA
CCTTATATTGTCCTGAAGAAAGGTGATGACTTAAAAATCATGCTCAATAGGATTACACTGAGGC
CCAGCCTAGGTGATAATTTTGGAAGAGGATGCTGGAATTCTGAGGTCCCTGACAGAGCAGCTGT
ATTTTGGGCTGGAGACCTTGAGGCCCTGAAGGGCATCTTGTGGGCCCACCCCTATCTCTGTGCT
CCTCTGTGAGGCAGCCCAGGCTCCCTGTGCTGGCTTGGTATCCGGGCTCTGGGTCCCTGGTGAC
GGGGCATGTTGGAGGCTCCTTCCTGAGCCCCTGTTCTCCCTTGTCATTCAGTGAACTCTTCTGC
ACAAGTGACCCCCCCAGCCTTGAGCGGCATAGGTGAGTGCATCCTGTTTTGGTCATGGGCCATG
AGTGATGCAGCATGGGGTCCCTGACATTCTGCCTTTGGGCCACCAGGGTTTTCTCAGGCTGCCA
ACCTACTCCCCACCAGGGAGCGATGGTTTCTCAGGAGGGCTGCTTCCTTGGGAATCTGACCCAA
GGAGCAAGTCTCTCCTGAGGCCCTGCAGGGGTGACAGTGGTCCAGGCTGGAAAGGGTTCCTTCA
GCCCAATGTAGGGCAGCTCCCAGCTGAAACTCAGTGGGCAGGTCAGGCCTCGAGTATTGCCCAA
GGCACATGGGGGAGTGAGGCACGGGGGATACAGTGCGCCGTGGAGGTCACACTGGAGTTCTCCC
CTCCCTGGAGAGGGATGAGTGACTGGGTCCACACGGTCAGGCTGTGGAGATTCAAGGCATGCTC
CACACCCATGGCCTTGAATGGGACCAACCCTGTTGAGCAATAGGCCATCTTAGTGGCACCTCCT
TGAGCCTGGTGGGGGACGGCACTGTTTCAATGAGGTTGGGTCAAGAGTGAGCTCTGGCTGGCAC
CTGGTGGTGCTGCAGGCCCTGGGACTTGTGGACTTTGGGCCAAGGGAGCAGCTCTGATCCTGCT
GGGGACAAGCCTTCCTTGAGGTCCAGTGGTGAAAAGAGTGTCCTCCTGAGATGGCAAGGCCTGC
TGTCGAGGGACCTGTTCTAGGAATTTGCTGCCCAGGCTGCTTCTGAGCCTTGGGGTCACCTCAG
GGGGTGGCAGGGTCTTGAGCCCAAGAAAGGAAAAGACACGATGTGGTTCCTGATTGGCTGAGTG
TGACCCGGCTCTGTGTGTCAGAGTTGGGCAGGACATGGATTGAGACCCACCATAGAAAGCATGT
GATCCTGGAAGCCCCTAGGCTTGGAGAGGGCACCAGGGCCAGCGCCCACCACGCCTAGGTACGA
TCTCTCGGACTTCCAGGTCTGCAGCGGCTCTTGGCGTCTGGGCCAGCATGCTTGGTTGGCCAGG
GGCTGGTTGGTGGCCCTGTCTTGTGCCCTGTGTCCCCGGTATGGCCCTGGGGTTGTGTGTGGTA
GCAGGCCTGGGCAGTGGAGGTGCTCTTGGGGCCTGCAGTAGGGTGCTGCCCAGGCATCTTGCCC
GAGCCAGGTGACACGCTTGCCCTGTCTTTCCAGGGTTTGGCGGCAGGGAGGGCCAAGGGTGCCC
AGTGTCCGTGAGGGATCAGTATTCAGCATGGATCCCATCACATGAGTATCCCTTAGCCCCTTCT
CTACATTGATGTGTGAGGCCCTGGTTAGACACATTTCCTCCTCCTGTCCCTCCAGATGGTAAGC
CTGGAGGAAGACTTGTGTTGGGCCCACAGGCCCCAGGCCAATGTCTGTCAGCCAGGTGCTCAGC
CTCTGGTGCGCTGAAACTCTGACCCTTCCTGGCACACTGGTCTCCTGCACTGAGCTGGGGTAAG
CACATCTGGGTTGCACTGGATGTTTGTGTGGGGAGGGGGTTCCCTAGGTTGGGTCAATGATGAG
AACCTGATATTGCCCTGAAGAGAGATGATGACTTAAAAATCATGTTCAATAGGATTACGCTGAG
GCCTAGCCTAGGTGAGAATTTTGGAAGCAGATGCTGGGATCCCAAGGTCCCTTGCAGGGCCATC
GTATGTTGAACTGGAGACCTGGAGGCTCTGAAGGGCATCTGGAGGTCGTGCAACCTGTCTCTGC
ACTCCTCTGTGAGGCAGCCCAGGCTCCCTGTGTGGGGTTGGTGTTGGTGCTATGGTTCCCTGGG
GGTGGGGAACATTGGTGGCTCCCTCCAGAGCCCCTGTTCTCACCTTGTGCCTTTCAGATTTTCC
ACCTAGGAGGTCCACCTGGCATTGACTGACATAGGTGAGTGGATCCTGCTGGGGTCATGGGTCA
TGAGCCAGACCATGGGGGGATCCCAGAGATTCAGTCTTTGGGCAACAGTTTTCTCAGGGAGCAG
ACCTGAATTTCCACCTGGGAGGGAAGGGTTCTCCTGGAGGGCGACTTTTTTGGGAATCGGACCC
AAGGAGGATGCCTTCCCTGAAGCCCTGCCGGGGTGATGGTGGTCCAGGCAGGAAAGTTCCTTCT
GCTCAATGCAGGGCAGCACCCAGCTGAGACTCGGTGCTCCGGTAAGGCCTCCAGAACTGCCCAG
GTCACATAAGGGGGGCCAGAGGGGCTACGAGGTGCCACGGGGTAACAGGGGAGCTCTTGGTCCC
CAGAGTGTGGTGCATGAATGGGCCCATTCCGTTGGGTCTCAGAGATTTAAGGATTGCACCACAC
CATGGCCCTGAATGCAACCGTCCCTGTTGATCAATGGGCCATCCCATGGGTCTCCTGGAGCCTG
GTGGGAGGCAGTGTTGTTTTCCTGGGGTTGGGTCAAGGCGGAGCTCTGGCTGGTACCTGGTGGT
GCTGCAGGTCAGCCCTGGGACTTGTGGGCTTTGGGCCAAGGGGCATCCCGCATCCCGCTGGGGG
CCACCCTTTCTCCATGGTCGAGTGTCGTGAAGGGTGGCCTCCTGAGTTGGGAAGGCTTGCTATT
GAGGCACTTGTACTGTGAATTTGTTGCTCAGGCTGCTTGGGAGCCATGGTGTCACCTCCAGGGT
GGCAGGGTCTTGGGACCAAGCAAAGGAGTGGGCACCATGTCACTGCAGATAGGCCATGTGTGAC
TGGCTCTGTGTCTGGGAGATATGTGGAGTATGAATGGGGACCCACCATTCATAGAGAAAGCAAG
TGGCCCTGCAGCCCGAGGCTATGATTTCCGGTGCACACTTGGAGAGGGTGCCTTTACACCCAAG
TACCAGGCTCAGAGGTAGGCTATGCCACTATCAGGTCTGCAGTGGCACGTGGCATCTGGGCCAG
CCTCCTTGGTTGGCCATGACCTGGCTGGTGGCCTGGGCCTGTGCCCAGTGTTCCCAGGGTAGTT
CTAGGGTGCTGCGGGGGAGCTGTTTTGGCTAGTGGAGGTGCTCTTGGGTCCTGCAGTACAGCAC
AGCCTGGGCGTTTTGCCTGAGCCCAGTGACCTGCCTGCCTTGCCCTTCCCAGGATCGGTGGTAG
GGAAGACCAGGGGTGCCCGGCATAGGTGATGAGTCAGTATTTCACATGGATATCATCACATGTG
TCCATGGAGGGGGAGCAAACATGAGCTGGCACCCGCCACCCCCTTCTCCACATGAAGGGTGGTC
ACACGTTCTCCATCTGTTCCTCCAGATGGTGAGACTTCTGTCGGCCTCGCTTTTCCTGGGCCAG
TGTCCATCAGCCAGGTGCTCAGCCCCAGTGCACTGAAGCTCCAGCCCTTCCTGGCGCCCTGGAC
TGAGCTGTGGTGAGCACATCCGGGTTCCACTGGATGCGTATGTGGGAAGGGGGGTGCCCTGGGT
TGGGTCAATGATGAGAACCGTATATTGTCCTGAAGAGCGGTGATGACTTAAAAATAATGCTCAA
TAGGATTACGCTGAGGCCCACCCTAAGTGAGAAATTTGGTAGGGGATGTTGGGATCCCGAGATT
TCTGGCAGGGCCACTGTAGTTTGGGTTGGAGGCCTGGAGGCCCTGAAGGGCATCTGGAGGTGGC
TCAACTCTTTCTCTGTGCTCCTCCGTGTAGCAGCCCAGGCTCCCTGTGCGGGCTTGGTGTTGTT
GCTTCTGTTCCTTGGGGGCAGGGCATGTGGGTGGCTCCTCCCTGAGCCCCAGTTCTCCCCTTGT
GTCTTTCAGTGAGCTCTTCTGCCAAGGGGGTCCTCCTGGCATTGACCAACATAGGTGAGTGGAT
CCTGCTGCTGTCATGGTCCGTGAGCCAGGCTGCTTGGGGTTGCCAAGGGTCAGACTTCGGGCCA
TGAAGGTTTCCTCAGGGAGCTGACCCTAATTCCCATGAGGGAGGCATGGTTTCTCCAGGAGGTC
AGCTTCCTTGGGAATCTGTGACTTGACAGGGTTGAGTCGCAGAGGTTCAAGGATCTCATCCCAC
CTGTAACCCTGAATGGGGCCCACCCTGTTGAGCAACTGGCCATCCCAGTGGCACATATTGAGCC
TGGTGGGGGGTGGTGCAGTTTCAATGGGGTTGGGTCAAGGCTGAGATATGGCTGGTGCCTGGTG
GTGCTGCAGTCCAGCCCTGGGATCCGTGGGCTTTGGGCTGAGGGGGAAGCCTGCATCCTATTGG
GGGTAACCTGTTCTCCAAATTTGAGTGATTGGTGGGAAGGCCTACTGTCTGAGGTCCTGTACTG
GGAATTTGCCACCTCAGCTTCTTCTGAGCCATAGGGTCACCTCCAGGGTCATGGGGTCTTGGGA
CCAAGGAAAAGAGAAGATACCATGTGGCCCTTGATTGGCCGAGTGTAGCCCAGCTCTGTGTTTG
GGAGGTGTGTGGGATGTGGATAGGGTCCCACCTGTGAAAGCATGTGGCCCGGGTCCCCAGGCTG
TGATTTTCAGTGCACACTTGGAGAGGGCACCTTGGTGGGTGCCCACCATGTCCAGGGGCAGGCT
ATGTGACTGCCAGGTTTGCAATGGTGTGTAGCTTCCTTCTACCCTCCTTGGTTGGCTGGGAGTG
GGTTGGTTGCTGTGGCCCATGCCCAGTGGTCACAGGGCAGGCAGACTGCCTTGTTTTTTCAGGG
TATGCTGACAGGGAGGGCCATGGGTGCCCTGCATTCATGATGAGTCAGTGTTCCACATGGAACC
CATTATGTGGGTATCTCTTATTCCTTCTACATATGGAGATGTGAGGCCCTATTTGGACACATGT
CCTCCTCCTGTACCCCCAGGTGGTGAGCCTGGAGGAAGACTTGCATTGGGCCCAATGGCCCTGG
ACCAGTGTCTGTCAGCCAGGTTCCCAGCCCCCGATGCGCTGAAGCTCAAGCCTTTCCTGGCACC
CTGGTCTCCTGCACTGAGCTGTGGTGAGCATATCCTGGTCCTGCTGGATGCATGCGTGGGGAGG
GGTTGTCCTAGGTTGGGTCAATGATGAGAACCTTATAATGTTCTGAAGAGAGGTGATGACTTAA
AAATCATGCTCAATAGGATTACGCTGAGGCCCAGCCTAGGTGATAATTTTGGAAGAGGACACTG
GGTCCCGAAGCCCCCTGCAGGGCCCCTGTGTTTTGGGCTGGAGACCTTGAGGCCCTGAAAGGCA
TCTGGTGGACCCAACTCTATCTCTGCCCTCCTCAATGAGGCAGCCCAGCCTCCCTGTACTGGCT
TGGTATCCGGGCTCCGGGTCTCTGGGGACGGGGCACATTGGAGGCTCCTTCCTGAGCCCCCATT
CTCTCCTGTGTCTTTCAGTGAGCTCTTCGGCCCAAGTGGTCCCCCCGGCCTTAAGCGGCATAGG
TGAGTGCATCCTGTTTTGATCATGGGCCATGAGTGTTGCAGCATGGGGTCCCCGACATTCAGCC
TTCAGGCCACTAGGCTTTTCTCAGGCTGCAAACCTACTCTCCACCAGGGAGCGATGGTTTCTCA
GGAGGGCCACTTCCTTCGGAATCTGACCCATGGAGCACGCCTTTCCTGAAGCCCTGCAGAGGTG
ACAGTGGTCCAGGCAGGAAGGGGTTCCTTCAGCCCCAATGTAGGGCAGCTCCCAGCTGAAACTT
GGTGGACAGATCAGGCCTGGAGTACTGCCCAGGGCATATGGGGAGGAGGGACACAGGGAATGTG
GCGCACCCTAGAGGTCACACTGGAGTTCTGTCTCCCTGGAGAGGGGTGAGTGATTGGGCCCACA
TGGTCAGGCTGTGGAGATTCAAGGAATGCTCCACACCGATGGCCTTGAATGGGACCAACCCTAT
TGAGCAATGGGCCATCTTAGTGGCACCTCCTTGAACCTGGTGGGGGATGGCACTCTTTCAATGA
GGTTGGGTCAAGGCTGAGCTCTGGCTGGCACCCAGTGGTGCTGCAGGCTGGCCCTGGGACTTGT
GGGCTTTGGGCCAAGGGGGAAGCTTCCATCCTGCTGGGAACAACCCTTCCTTGAGGTCCAGTGG
CAGGAAGGGGGGCTTCCTGAGGTGGAAAGCCCTACTGTTGAGGGACCTGTCCTGGGAATTTGCT
GTCCAAGCTGCTTCTGAGCCATGGGGTCACCTCTAGGGTGGTGGGGTCTTGAGCCCAAGGAAAG
GAGAGGACACCATGTGGTTCCTGATTGGCTGAGTGTGGCCCGGCTCTGTGTGTCGGAGTTGTGC
AGGGTATGGATTGGGACCCACCAGTGAAAGCACATGGCCCTGGAGGTCCGCAGGCTTGGAGAGG
GCACCAGATCTGGCACCCACCATGCCCAGGTACAGGCTCTGGGACTGCCAGGTTGCAGCAGCTC
TTGGCATCTGGGCCACCCTCCTTGGTTGGCCAGGAGCTAGTTGGTGGCCCTGTCCCATGCCCCA
TGTCCCTGGTATGGCCATGGGGTCATGCAGAGGAACCGGCCCTGGCATGGAGGTGCTCTTGCAA
CATGCAGTAGGGTGCATCCCAGGCATCTTGCCTGAGCCAGGTGACCCGCTTGCCCTGTCTCTCC
AGGGTTTGGTGGCAGGGAGGGCCAAGGGTGCCCCGTGTCCATGTTGGGTCAGTGTTCAGCATGG
ATCCCATTGCATGAGTATCACTTAGCTCCTTCCCAGCATTGATATGTGAGGCCCTGGTTGGACA
CTTGTCCTCCTGTCCCTCCAGATGGTGAGCCTGGAGGAAGACTCGTGTTGGGCCCACAGGCCCC
AGGCCAGTGTACGTCAGCCAGGTGCTCAGCCCATGGTGTGCTGAAGCTCTGACCCTTCCTGACT
CCGTGGTCTCCTGCACTGAGCTGTGATGACCATATCCAGGTCCTGCTAGATGCATGCGTGGGGA
GGCAGGGTGCCCTGGGTTGGGTCAATGATGAGAACCTTGTATTATCTTGAAGAGAGGTGATGAC
TTAAAAATCATGCTCAATAGGATTACACTGAGGCCCAGACTAGGTGAGAATTTTGGAAGAGGAT
GCTGGGATCCTGAGGTCCCCAACAGGGCCACCATATTTTGGGCTGGAGACCTGGAGGTCCTGAA
GGGCATCTGTAGGGGGCCCAACCCTGTCTCTGCACTCCTCCCTGAGGCAGCCCATGCTCCCTAT
TCGGGATCGGTATTGGTGCTCCACTTCCCTGGGGGCAGGACACGTGGGTGGCTACTCCCTGAGC
CCCCGTTCTCCCCTTGTGTCTTTCAGTGAGCTCTTCCAAACAGGTGGGCTCCCTAGCATTGACC
GACAGAGGTGAGTGGATCCTTCTGGGATTATGGGCCATGAGCCAGGCCATGTGGGGTCCCCAAG
GTTCAGCCTTTGGATCCCGAGAGTTGTCTCAGACAGCCAACCTGATTTGCCACCTGGGAGAGAT
GGCTTCTCCAGGAGGGTGGGTTTTTGGGAATTGGACCCAAGGAGGACGCCTTCCCTGAAGCCCT
ACTGGGATGATGGTGGTCCAGGCAGGAAAGGGTTCCTTCACCCAGCTGAGACTCGGTGTTCCAG
TAAGGCCTCAAGGACCTCCCTGCCACATGAAGGGGCCCAGAGGGGCTACGAGGCACCACAGGAG
TGACAGGGGAGCTCTTGGTCCCCAGAGTGTGGTACATGAATGGGCCCATGGGGTTGAGTCGCAG
AGATTCAAGGATGCTGCACCACCCATGGCCCTGAATGTGACTCTCCTCCTGGAGCCTGGTGGGG
GGTGGCGTTTTTTCCCTGGGGTTCGGTCAAGGTGGAACTCTGGTCAGTACCTGGTGGTAATGTA
GGTCAGCCCTGGGACTCGTGGGCTTTGGGCCAACGGACAGCCCACATCCCTCTGGGGGTCACCC
TTTCTCTGAGGTCGAGTGTCGTGAAGGGTGGCCTCCTGAGGTGGGAAAGCCCAATGTCGAAGGA
CCTGTACTGAGAATTTGTTGCTTAGGCTGCTTAGGATCCATGTTGTCACCTCCAGGGTGGTGGG
ATCTTGGGACCAAGCAAAGGAGAGGGCACCATGTGGCTGCCAGTGGCTATGTGTGACTGGCTCT
GTGTCTGAGAGGTGTGTGGATGGGGACCCACCAGAGAAAGCAAGTGGCCCTGCAGCCCCAAGAT
GTGATTTCTGGTGCAGGCTTGGAGAGGGTGCCTATACCTGCTCCCACCACGCCCAGGGGCAGGC
TTTGCCACTGCAGGTCTGCAGTGCCACGTGGCATCTGGGCCAGCCACCTTGGTTGGCCGTGACC
TGGCTGGTAGCCCGTGCCTGTGCCCAGTGTTCCCAGGGCTGCCCTGGGGTCTTGCTGGGGAGCT
GTTTTGGGCAGTGGAAATGCTCTTGGGGCCTGCAGTATGGCACAGCCTGGGGGCATTTTGCCTG
AGCCCAGTGACCTGCCTGCCTTGTCCTTCCTGGGTTCGGTGGCAGGGAGGGCCAAGGGTGCCCT
ACATTGGTGACAAGTCAATATTTTGCATGGATCTCATCACGTGTCCATGGAGGTGGGGGCAAGC
GTGAGCTGGCACGCACCCCATTCTCCCCATGGAGGGTGGTCACATGTACTCCACCTGTGTCCCT
AGATGGTGAGACTGGAGGAAGACTTCCATTGGGCCCACTTTTCATGGGACAGTGTCCATCAGCC
AGGTGCTCAGCCCCCAGTGCATTGAAGACCCAGCCCATCCTGGCACCCTAGTCTCCTGCACTGA
GCTGTGCTGAGCACATCTGGCTTCCACTGGAAGTTTTTTTTGCGAGGAGGGGCTTCCCTGGGTT
GGGTCAATGATGAGAACCTTATATTGTCCTGAAGAGAGGTGATGACTTAAAAATCATGCTCAAT
AGGATTACGCTGAGGCCCAACCTAGGGGAGAATTTTGGAAGAGGATTATGGGATCCCAAGGTCC
CCAACAAGGCCACTGCATTTTGGGCTAGAGACCTGGAGGCTCTTAAGAGCATCTGGAGGTGGCC
CAACCTGTCTCTGCTCCTTCGTGATGCAGCCCAGGCTCCCTGTGTGGGATTGGTGTTGGTGCTC
CATTTCCCTGGCATGGGGAATGTTGGTGGCTCCTTCCTGAGCTCCTGTTCTCGCCTTGTGTCTT
TCAGTGAGCTCTGCCACCCAGGACATCCCCCTGGCATTGACTGGCATAGGTGAGTGGATCCTGC
TGGGGTCATTGGTCATGAGCCAGACCATGTGGAGTCCCCAAGGTTCAGCCTTTGGGCCACAAAG
GTTTTCTCAGGGAGCAGACCTGAATTTCACCCGGGAGGGAAGGGTTGTCCTGGAGGGTGGCTTT
TTTGGGAACCTAACCCAAGGAGGACCCATTCCCTGAAGCCCTGCAGGGGTGATGGTGGTCCAGG
CAGGAAAGGGTTCCTTCTGCTCAATGCAGGGCAACACCCAGCTAAGATGCGGTGATCTGGTAAG
GCCTCCAGAACTGCCCAGGCCATATGAGGGGGGCCAGAGGGGCTACAGGGTGCCACAGGGGTAA
CAGGGGAGCTCTTGGTCCTTGGAGTGTGGTGCCTGAATGGACCCATGGGATTGGGTTGTGTAGA
TTCAAGGATCACACCCTACCCACAGCCCTGAATGTGACTCTTCCTCTTGAGCAATGGGCCATCT
CAGTGGGTCTCCTGGAGCCTGGTCGGGGGGTGGTGTTGTTTCCCTGGGGTTGGGTCTAGGTGGA
GCTCTGGCTGGTACCTGGTGGTGCTGCAGATCATCCCTGGGACTTTTGGGCTTTGGGCCAAGGG
ACAGCCCGCATCCCGCTTGGGGTCAGCCTATCTCCATGGTCAAGTGTCATGAAGGGTGGCCCCC
TGAGGTGAGAAGGCCTACTGTCAAGGGATCTGTCCCAGGATTTTGTTGCTCAGGCTGCTTCTGA
GCCATGGCGTCACATCCACAGTGGCAGGGTCTTGGGCCTGAGCATAGCAGAGGGCACCATGTCG
TTCTGGATTGGCCAAGTATGGCCTAGCTCTGTGTGCGGGAGGTGTGCAGAGCATGGACGGTGAC
CCACCAGAGAAAGCACGTGGCCCTGCATCCCAGGCTGTGATTTCCAATGCACACTTGGAGAGGG
TGTCTTGATTTGTGCCCACCATGCCCAGGGGCAGACTACGTGATAGCCAGGTCTGCAGCCTGAG
CCACGTGTGGCATCTGGGCCAGCCACCTTGGTTGGCCGTGAGCTGGCTGGTGGCCTTGGCCTGT
GGCCAGTGGTGCCAGGGCGGCCCTGGGGTCGTGTGGGGGAGCCATGTTGGGCAGTGGAGGTGCT
GTCAGGGCCTGCAGTACAGTGCAGCCTGGGCATTTGGCCTGAGCCCAGTCACCCGCCTGCTCTG
TTCCTCCAGGGTTTGGTGGCGGGGAGGGCCAAGGGTGCCCCGCATCCGTGACGACTCAGTAATT
CACATGGATCACATCACATTTTTCCTTGGAGGGCAAGCAAATGTGAGCCCCCACCCCCCACTGC
CTTCCCCACATGGAGGTCATTTTGGACACATATCCTCCTCTAGTCCCCCTAGATGGTGAGACTG
GAGGAAGACTTGCATTATGCCCGCTGTTCCTGGGCCAGTGTCAGTCAGCCAGGTTCCCAGCTCC
AAGTGTGCTGAAGCTCAGGCCCTTCCTGGCACCCTCAGCTCCTGCACTGAGCTGGGGTTACATC
CGGGTTCCACTGTTTGCATGCAAAGGGAGGGGTGTACCCTGGGTTGGGCTGATGATGAGAACCT
AGCTAGGTGAGAATTTTGGAAGATGAGGCTGGGATCCTGAGATCCCCGGCAGGGCCACACTTAT
TTTGGGCTGGAAAATTTCAAGACCTGAAGCGCATCTTGTGGGGGGGCAATCCTGTCTCTGTGCT
TCTCCGTGAGGCAGCCCAGGCTCCCGTTGCGGGCTTGGTGCCCATGCTTCGCTTCCCCAGGGGT
GGGGCAATTTGGCATCCTCTTCCTGAGCCCTCCTTCTTCCCTGTGTCTTTCAGTAAGTTCTTCT
TCCCAGGTGGGGCCCCTGGCTTTGAGCGGCATGGGTCAGAGCATCCTGTCAGGGTCATGGGCCA
TGAGTGAGGCAGCGTCGGTTCGTCGAGAGTCAGCCTTTGGGCCACTGGGGTTTTCTCAGGGAGC
CAACAGGTTACCCATCAGGAGGGATGGTTCCTCCAGGAGGGTCGCTTCCTTGGGAATCTGACTC
TAGGAGCATGCATTTCCTGAAGCTCTGCAGGGTGATGGTGGTCCAGGCGGGAAAGGGTTCCTTC
AGCCCAACATAATGCAGTTCCCAGCTGAAACTCGGTGGCAGATCAGGCTCAAGGACTGCTCGGG
CCACATGATGGGGGCCACAGGGGATTTGGGGTGCCCTGGGGGTCATGCTGGAGTTCTTGCCCCA
AGAGGGGTGGGTGATAGGGCCCATGCGATCAGGCTGCGGAGATTCAAGGAATGCTACCCACCCA
TGGCCCTGAATGGGACTGACCCTGTTGAACAATGGGCCATTTTAATGGTGCCTCCTTGAGCCTG
GTAGGTAGTGGCATTGTTTCAATGCGGTTGGGTCAAGGCTGAACTCTGGCTGGTGCCCGGTGGT
GCCCACATGGGCTTTGGGCCAAGGGGGCAGCTCATATCCCTCTGGGGTCACCCCTTCCTTGAGA
TTCAGTGGCAGGAAGGGTGTTCTCCTGAGGTGGAAGACCTGCTGTCAAGGGACTTGTCCTGGGA
ATTTGCCACCTAGGCTGCTTCTGAGCCATGGAGTCACCTCCAGGGTGGCAGGGTCTTGAGCCCA
GGGAAAGGAGACACCATATGGTTTCTGACTGGGAGTGTGGCCTGGATCTGTGTGTGGGAGGTGT
GCAGGGCATGGATGGGGACCCATCAGAGAAAGCATGTGGCCCTGTAGGCCCCCAGGCTTTGATT
TCTGGTGTGTGCTTGCAGTGGGCACCAGGGCAGCCCTACCATGCCCAGGGGCAGGCTCTGTGAC
TGCCAGGTCTGGAGTGGCTCCTGGCATGAAGACCACGCTCCTTGGTTGGCCATGAGCAGGTTGG
TTGCCCTAGCCTGTGCCCTGTGGTCCTGGTGCAGCCCTGGGGTTGTTTGGGGAGACAGCACCGG
CAGTGGAGGTGCTCTCAAGGCCTGCAGTAGGGCGCAGCCCAGGTGTCTTGCCCGAGCCCAGTAA
CCCACTTGCCCTGTCTTTCCAGGGTTTGGTGGCAGGGAGGGCCAACTGTGCCCCGCATCTGAAA
TGGATCAGTGTTCTGCATGGATCCCATCACAAGGGTATCCCCTACCCAACTTCTGTGCATTGTT
ATGTGAGGCCCTGGTTGGACACATGTCCTCCTCCTGTCCCTGCAGATGCTGACCCTGGAGGAAG
ACTTGTGTCGGGCCCCCAGGCCCCAGGCCAGTGTCCATCAGCCAGGTGCTCAGCTCCCAGTGCG
CTGAAGCTCAGACCCTTCCTGGCACCCTGGTGTCCAGCACTGAGCTGTGGTGAGCCCATCCGGG
TCCTGCTGGATGCATGAATGGGGAGGGGCCTGCCCAGAGTTGGGTCAATGATGAGAACCTTACA
TTGTCCTGAAGAGAGATGATGACTTAAAAATCATGCTCAATAGGATTACGCTGAGGCCCAGCCT
AGGTGAGAATTTTGGAAGAGGACGGCTGGGATCCTGAGATCCCAGTCAGGGTCACTGTATTTTG
GGCTGGAGACCTGGAGGCCCTAAAGGGCATCTGGAAGGGGGCCCAGCCCTGTCTCTGTGCTCCT
CCGTGAGGCAACCCACACTCCCTGTGTGGGCTTGGTGTTGGTGCTCTGGTTCCCCAGGGACAGG
GCGGGTTGGTGGCTCCTTCCTGAGCCCCTATTCTCCCTTTGTGTCTTTCAGTGAGCTTTTCCAC
CCAGGTGGGCCCCCTGGCATTGACCGACATAGGTGAGTGCATCCTACTGGGATAATGGGCCACT
AGATAGTCTGTGTGGGGTTGCTGAGGTTTAGCCTTTGGGCCACAAGGGTTTTCTCAGGCAGCCA
ACCTGAATTCCCACCTGGGAGAGATGGGTTCTCCAGGAGGCAGCTTTTTTGGGAATCAGACCCA
AGGAGGACACCTTCCCTGAAGCCCTGCAGGGGTGATGGTGGTCCAGGCAGGAAAGGGTTCCTTC
TGCCCAACTCGAGGCCCAATGCAGGGCAGTGCCCCCCTGAGAATTGGTGCTCTGGTAAGGGCTC
GAGGACTGCCCAGGCCACATGAAGGAGGTCAGAGGGGCTATGGGGAACCATGGGGGCAACAGGG
GAGCTCTTGGCCCCCGGAGTGTGGTACATGAATGGGCCCATGGGGTTGGGTCACGGAGATTCAA
GGATTGCATCTCACCCACGGCCCTGAATGCGACCATCCCTGTTGAGCAATGGGCCATCCCAGTG
GGTCTCCTGGGGCCTGGTGTGGGGTGGCATTGTTTTCCTGGGGTTGGGTCAAGGCGGAGCTCTG
ACTGGTACCTGATGGTGCTGCAGGTCAGCCCTGGGACTCGTGGGCTTTGGGCCAAGAGGCAGCC
CGCATCCTGCTGGGGGTCGCCTTTTCTCCGAGGTTGAGTGTCGTGAAGGATGGCCTCCTGAGGT
GGGAAGGCCCACTGTCAAGGCACCTAGACTAGGAATTGTTCCTCAGGCTGCTTGGGAGCCATGG
TGTCACCTCCAGGGTGGCGGGATCTTGGGACTAAGCAAAGGAGGGGACACTACGTGGCTCCCAA
TTGGCCATGTGTGGCTGGCTCTGTGCCTGGGAAGTGTGTGGAGTATGGATGGGGACCCACCAGA
GAAAGCAAGTGGCCCTGCAGTCCCAGAATGTAATTTCTGGTGTAGGCTTGGAGAGGGTGCCTAT
ATGCATGCCCACCATGCCCAGGAGCAGGCTATGCCACTTCAGGGTCTGCAGGGGCACCTGGCAT
ATGGGCCAGCCTCCTTCGTTGGCTGTGACCTGGGTGGTGGCCTGTGCTTGTGCCCAGTGGTCCC
AGGGTAGCCCTGGGGTCGTGCAGGGGAGCCGTTTTGGGCAGTGGATGTGCTCTCGGGTCCTACA
GTACAGCACAGCCTGGGTGTTTTGCTCAAGCCCAGTGACCTGCCTGCCTTGTCCTTCCCGGGTT
CAGTGGCAGGGAGAGCCAAGGGTGCCCCACATTGGTGACGAGTCAGTATTTTGCATGGATCTCA
TCACACGTGACCATGAAGGACGAGCAAGCATGAGCTGGCACCAGCCACCCCCTTCTCCGCGTGG
AGGGTGGTCACATGTCCTCCACCTGTGCCCCCAGATGATGAGACTGGAGGAAGACTTCCATTGG
GCCCACTTTTCCTGGGCCAGTGTCCATCAGCCAGGTGCTCAGCCCCTGGTGTACTGAAGCTCCA
GCCTTTCCTGGTGCCCCAGTCTTCTGCACTAAGCTGTGGTGAGCACATCCGGGTTCCACTGGAT
TTTTGTGCGGGGAGGGGGTTTTCTACGTTGGGTCAATGATGAGAATCTTATATTGTCCTGAAGA
GAGGTGATGACTTAAAAATCATGCTCAATAGGATTACGCTGAGGCCCAGCCTAGGTGAGAATTT
TGGAAGGGGATTATGGGATGCTGAGGTCCTCAGCAAGGCCACTGTATTTTGGGCTAGAGACCTG
GAGGCTCTGAAGGGCATCTGGAGGTGGCCCAACCTGTCTCTGCGCTCCTCCATGAGGCAGCCCA
GGCTCCCTGTGCAGGGTTGGTGTTGGTGCTCCGGTTCTCTGGTGTGGGGAATGTTGGTGGCTCC
TTCCTGAGCCCTCATTCTCACCTTGTGTCTTTCAGTGAGCTCTTCCACCCAGGAGGGCCCCTTG
GCATTGACTGGCATAGGTGAGTGGATCCTGCTGGGGTCATTGGTCATGAGCTAGACCGTGTGGG
GTCCCCGAGGTTCAGCCTTCAGTCCACAAGGGTTTTCTCGGGGAGCAGACCTGAATTTCCACCT
GGGAGGGAAGGGTTCTCCTGGAGGGTAGTTTTTTTGGGAATCTAGCCCAAGGAGGATGCCTTCC
CTGAAGCCCTGCAAGGGTGATGGTGGTCCAGGCAGGAGAGGGTTCCTTCTGCCCAATGCAGGAC
AGCTCCCAGTTGAGACTGAGTGCTCCAGTAAGGTCTCCAGGACTGCCCAGGCCACATGAAGGAG
GCCAGAGGGGCTATGGAGCACCACTGGGGTAATAGGGGAGCTCTTGGTCCCTGGAGTGTGGTGT
GTGAATGGGGTTTGGTCACGGAGATTCAAGGATCACACCCCACCCATGGCCCTGAAAGTGACCA
TCCCTATTGAGCAATGGGCCATCCCACCGGGTCTCCTGGAGTCTGGTGCAGGGCGACGTTTTTT
CCCTGGGGTTGGGTCAAGGTGGAGCTCTGGTACCTGGTCATGCTGCAGGTCAGCCTTGGGACTC
GTAAGCTTTGGGCCAAGGGACAGCTGGCATCCTGCTGGGGGGTCACCCTATCTCTGGGGTCGAA
TGTCACGAAGGGTGGCCTCCTGAGGTGGGAAGGCCCATTGTCGAGGGACCTGTCCCAGGATTTT
GTTGCTCAGGCTTCTTCTGAGCCATGGTATCACCTCCAGGGTGACAGGGTCTTGGGCCCAAGCA
AAGGAGAGGGCACCATGTCGCTCTGAGTTGGCCAAGTGTAGCCTAGCTCTGTGGTGGGAGGTGT
GCAGAGCATGGCTGGTGACCCACCAGAGAAAGCACGTGGCCCTGCAGCCCCAAGCTGTGATTTC
CAGTACATGCTTAGAGAGGGCATCTTGATGTGAGCCCACCACACCCAGGGGCAGACTACGTGAC
TGCCAGGACTGCAGTGGCACATGGCATCTGGGCCAGCCTTCTTGGTTGGCCATGACCTAGCTAA
TGGCCCTGGCCCGTGCCTTGGGGTCATGCAGGGGAGCCCTCTTGGACAGTGGAGGTCCTCTCAG
GGCCTGCAATATGGCGCAGGCTGGGCATTTGGCCCGAGCCCAGTGACCTACCTGCCCTGTCCTT
CCAGGGTTGGGTGGCAGGGAGGGACAAGGGTGCCCCATGTCAGTGACAAGTCAGTATTTCACAT
GGATCTCATCACATGTGCCCATGGAGGACGAGCAAGCGGGAACTGGCACCCGCTACTTCCTTCC
CTACATAGAGGCCATTTTGGACACATGTTCTCCACCTCTCTCCCAGATGGTGAGACGGGAGGAA
GACTTGTGTTGGGCCTGCTGTTCCTGGGCCAGTGGCCATCAGCCTGTTGCCCAGCCCCTGAGGC
GCTGAAGCTCAGGCCCTTCCTCACACCCTGGTCTCCTGCACTAGCTGTGGTGAGCACATCCGGG
TCCCGCTGGATGCATGCAAGAGGTGGTTGGTCTCGTGGGTTGGGTCGATGATGAGAACCTTATA
TTTTCCTGAAGAGAGGTGATGACTTAAAAATCATGCTCAATAGGATTACGCTGAGGCCCAGCCT
AGGTGAGAACTTTGGAAGAGGATGCTGGGATCCTGAGATCCCAGGCAGGGCCACTATATTTTGG
GGTGGAGAACTCAAGGCCCTGAAGGGCCTCTCAGGGGGTGCCACCCTGTCTCTGTGCCCCTCTG
TAAGGCAGCCCAGGCTCCCTGTGTGGGCTTGGTGTCTGGTCTCTGGTTCCCCGGGGGCGGGGCA
TGTTGGTGGCTCCTTCCTGAGCCCGCTTTCTCCCCCATGTCTTTCAGTGAGCTCTTCTGCCCAG
GTGGGGCCCATGGCCTTACACGACATAGGTGAGTGTATCCTGTTGGGGTCATGGGCCATGAATG
AGGCAGCATGGGGCCATTTAGGGTCAGCCTTCGGGCCACTAGAGTTTTCTCAGGGTGCGACCGG
GGAGGGAACATTTCTCCAGTAGGGTCACTTCCTTGGAAATCTGATCCTAGGAGCACGCATTTCT
TGAAGTCCTGCAAAGGTGATGGTGGTCCAGACAGGAGAGGGTTCCTTCAGCCCAACATAGCGCA
GCTCCCAGCTGAAACTCGGAAGGCTGGTCAGGCCTCGAGGACTGCCCAGGCCACATGAGGTGGG
CAACAGGGGCTCTGGGGCAACCTGGGGGTCACGCTGGAGTTCTCCCTCTCTGGACAGTGGTGGG
TGATTGGGCCCACGCAGACAGGCCCCGGAGATTCAGGACTGCACCCCATCCATGGCCCTGAATG
GGACCGACCCTGTTGAGTAATGGGCCATCTCAGTGGTACCTCCTTGAGCCTGGTAGGGGGAGAC
ACTGTTTCAATGCAGTTGAATCAAGGCTGAGCTCTGGCTGGTGCCTGGTGGTGCTGCAGGCAGC
CCTGAGGGTTGTGGTCTTTGGGACAAGGGGTAGCTCACATCCCACTGGGGTCACCCCTTCCTTG
AGATTCAGTGGCCAAAAGGGTGTCCTCCTGAGATGGGAAGTCCTGCTGTCGAGGGACCTGTTCT
GGGAATTTGCCACACAGGCTGCTTCTGAGCCACGGGGTCACCTCCAGGGTGGTAGGGTCTTGAT
CCCAAGGAAAGGAGAGGACACCATGTGGTTTCTTATTGACCAAGTGTGGCCTGGCTTTGTGTGT
GGGAGGTGTAACGCAAGATGAGGACCCACCAGAGAAAGCACGTGGCTCTGGAGGCCCCCAGGGT
TTGATTTCCGGTGTGTGCTTGGAGAGGGCACCAGGTTGGGCACCCACCACGCCCAGGGCAGGCT
CTGCGACTGCCAGGTCTGCATTGGCTCCTGGTGTCAGGGCCAGACTCATTGGTTGGCCATGAGC
AGGTTGGTTGCCCTGACCCATCCCCATGGTCCCAGTGTGGCCCTGGGGTTTTTCAGGGGAGCCG
GCTCAAGCAGTGGAGGTGCGCTTAGGGCCTGCAGTAGGGAACAGCCCAGGCATCTTGCCTGAGC
CCAGTAACCTGCTTGCCTTGTCTTTCCAGGGTTTGGTGGCCGGGAGGGCCAATGATGCTCTGCG
TCTGTGTTGGATCAGTGTTCTTCATGGATTCCATCACGTGGGTATCCCCTACCCACCTTCCCCT
TGCTCATACGTGAGGCCCTGGTTGGACACATGTCCTTCTCCTGTCACCCCAGATGGTGAGCCTT
GAGGAAGACTTGTATTGGGCCCCCACAGGCCCCAGGCCAGTGTCCGTCAGCCTGGTGCTCACCC
CCAGTGTGCTGAAGCTCAGACCCTCCGTGGCACCCTGGTCTCCTGCACTGAGCTGTGGTGAGCA
CATTCAGGTTCCGCTGGATGCATGCATGGAGAGGGGCTTGCCCTTGGTTGGGTCAGTGATGAGA
ACCTTCTATTGTCCTGAAGAGAGGTGATGACTTAAAAATCATGCTCAATAGGATTACGCTGAGG
CCCAACCTAGGTGAGTATGTTGGAAGAAGACACTGGGATCCCGAGATCTCCAACAGGGCACTGT
ATTTTGGGTTGGAGACCTGGAGGCCCTGAAGGGCATCTGGAAGGAGGCCCAACCCTGTCTCTGT
GCTCTTCCTTGGGGCAGCCCAGGCTACCTCTGCGGGCTTGGTGTTGGTGCTCCAGTTCCCTGGG
GCCAGGGAACATTGCTGGCTCCTTCCTGAGCCCCCATTCCTCCCTTGTGTCTTTCAGTGAACTT
TTCCTCCCAGATGGGCCCCCTGGCATTGACTGGCATAGGTGAGTGCATCCTGCTGGGGTCATGG
GCCATCAGCCAGGCCATGTGGGGTCGCTGAGGTTCAGCCTTTGGGCCACAAGGGTTTTCTCAGG
CAGCCGACCTGAATTCCCACCTGGGAGAGACGGGTTCTCCAGGAGGGCAGCTTTTTTGGGAATT
AGACCCAAGGAGGATACCTGAAGCCCTGCAGGGGTGACGGTGGTCCAGGCAGGAAAGGGTTCCA
TCAGCCCGGGCAAGGCAGCTCCTAGCTGACACTCGGTGTTCGGGTCAGTCCTTGAGGACTGCCT
GAACACGAGGGGGTCCAGAGGGGCTCTGGGTGCCACGGGGGTCACATGGGAGCTCTCAGTCCCT
AGAGTGTGGTGTGTGAATGGACCCTTAGGGTTAGGTCTTGGAGATAAAAGGATTGTGCTCCACC
CATGGCCTGGAATGTGACCGTCCCTGTTGAGTAATGGGCCATTCCAGTGGGTCTCCTGGAGCCT
GATGGGAGGTGGCGTTGTCTTCCTGGGGTTGAGTCAAGGCAGAGCTCTGGCCCATACCTGGTTG
TGCTGCAGGTCAGCCCTGGGACTCGTGGGCTTTGGGCCGAGGGGCAGCCCGCATCCCGCTGGAG
ATTACCCTATCTCTGAGATCAAGCGTCATAGAGGGTGACCTCCTGAGAAGGGAAGGCCCACTGT
CGAGGGACCTTTCCTAGGATTTTGTTGCTCAGGCTGCTTGGAATCCATTGTGTCACCTCCAGGG
TGATGGAGGTTTGGGCCCAAGCAATGGAGAGGGCACCATGTGTCTCCTGATTGTTCAAGTGTGT
CGGAGATATGCGGGGTATGGATGGTGGCCCACCAAAGAAAGCATGTGACTCAAGCTGTGATTTC
CAGTGCATGCTTAGAGAGGGCGCCTGTATGCGTGCCCCACATGCCCGGGGTAGGCTATGCAACT
GCCAGGTCTGCAGTGGCGCATGGCATCTTGGCCAGCCTTCTTGGTTGGCCATGACCTGGCTAAT
GACCCTGGCCTGTGCCCAGTGATCCCAGGGAAGCCCTGGTGTCATGCAGCGGAGGCCTCTTGGG
CAGTGGAGGTGCTCTTAAAGCCTGCAGCATGGCACGGCCTGGGCATTTATCCCAAGCCCAATGA
ACTACCTGCCCTGTCCTTCCAGGGTTCGGTGGCAGGCAGGGCCAAGGGTGCCCCGTGTTGGTGA
CAAGTCAGTATTICACATGGCTCTCATCACATGTGTCCGTGGAAGATAAGCAAGTGTGAACTGG
CACGTCCTACCTCCTTCCCTACATGGAGGTAACTTTGGACACATGTTCTCCACCTCTCTCCCAG
ATGGTGAGACCGGAGGAAGACTTGCATTGGGCCTGTTGTTCCTGGGCCAGTGGCCATCAGCCTG
TTGCCCAGCCCCTGATGCGCTGAAGCTCGGGCCCTTCCTCGCGCCCTGGTCTCCTGCACTGAGC
TGTGGTGAGCACATCCGGGTCCTGCTGGATGCATGCACGAGGTGATTGATCCCCTCAGTTGGGT
CGATGATGAGAACCTTATATTGTTCTGAAGAGAGGTGATGACTTAAAAATCATGCTCAATAGGA
TTACGCTGAGGCCCAGCCTAGGTGAGAATTTTGGAAGAGGATGCTGGGATCCCGAGATCCCTGG
CAGGGCCACTATATTTTGGGCTGGAGAACTTGAGGCCCTGAAGGGCATCTCAGGGGGGTCCAAC
CCTATCTCTGTGCCCCTCCATGAGGCAGCCCAGGCTCCCTGTGTGGGCATGGTGTCCGGGCTCT
GGTTCCCTGGGGGTGGGGCATGTTGGTGGCTCCTTCCTGAGCCTGCTTTCTCCCCCATGTCTTT
CAGTGAGCTCTTCTGCCCAGGTGAGGCCCATGGCCTTAAGCGGCATAGGTGAGTGTATCCTGTT
GGGGTCATGGGCCATGAATGAGGCAGCATGGGGCCATTTATGGTCAGCCTTCGGGCCACTAGGG
TTTTCTCAGGGTGCGACCGGGGAGGGAATGTTTCTCCAGTAGGGTCACTTCCTTGGGAATCTGA
CCCTAGGAGCATGCATTTCGTGAAGCCCTGCAAAGGTGATGGTGGTCCCGGCAGGAAAGGGTTC
CTTCAGCCCAACATAGAGCAGCTCCCAGCTGAAACTCAGCGGGCTGGTCAGGCCTCGAGGACTG
CCCAGGCCACATGAGGTGGGCAACAGGGGCTCTGGGGCAACCTGGGGGTCACGCTGGAGTTCTC
CCTCTCTGGACAGTGGTGGGTGATTGGGCCCACGCAGACAGGCCCCGGAGATTCAGGACTGCAC
CCCATCCATGGCCCTGAATGGGACCGACCCTGTTGAGTAATGGGCCATCTCAGTGGTACCTCCT
TGAGCCTGGTAGGGGGAGGCACTGTTTCAATGCAGTTGAATCAAGGCTGAGCTCTGGCTGGTGC
CTGGTGGTGCTGCAGGCAGCCCTGGGGGTTGTGGTCTTTGGGACAAGGGGCCAGCTCACGTCCC
TCTGGGGTCTCCCCTTCCTTGAGATTCAGTGGCCGAAAGGATGTCCTCCTGAGGTGGGAAGTCC
TGCTGTCGAGGGACCTGTTCTGGGAATTTGCCACACAGGCTGCTTCTGAGCCACGGGGTCACCT
CCAGGGTGGTAGGGTCTTGATCCCAAGGAAAGGAGAGGACACCATGTGGTTTCTTATTGACCAA
GTGGGACCTGGCTCTGTGTGTGGGAGGTGTAGGGCAAGATGAGGACCCACCAGAGAAAGTACGT
GGCCCTGGAGGCCCCCAGGCTTTGATTTCTGGTGTGTGCTTGGAGAGGGCACCAGGGTGGGCAC
CCACCATGCCCAGGGTAGGCTCTGCAATGGCCAGGTCTGCATTGGCTCCTGGTGTTAGGGCCAG
ACTCGCTGGTTGGCCATGAGCAGGTTGGTTGCCCTCACCCGTCCCCATAGTCCCAGTGCGGCCC
TGGGGTTGTTCGGGGGAGCCGGCCCGAGCAGTGGAGGTGCTGTTGGGGCCTGCAGTAGGGCATC
TTGCCCCAGCCCAGTGACCCACCTTCCCTGTCTTTCCAAGGTTTGGTGGCCAGCAGGGCCAATG
GTGCCCTGTGTCTGTGATGAGTCAGTGTTCTTCATGGATTCCATCACATGGGTATCCCCTACCC
ACATTCCCCTCGCTCATATGTGAGGCCCTGGTTGGACACATGTCCTTCTCCTGTCACCACCCCA
GATGGTGAGCCTGGAGGAAGACTTGTATTGGGACCCTACAGGCCCCAGGCCAGTGTCTGTCAGC
CTGGTGCTCATCCCCCAGTGCGCTGAAGCTCAGACCCTCCCTGGCACCCTGGTCTCCTGCACTG
AGCTGTGGTGAGCACATCCAGGTTCTGCTGGATGCATGCATGGGGAGGGCCTTGATTGGGTCAA
TGATGAGAACCTTATATTGTCCTGAAGAGAGGTGATGACTTAAAAATCATGCTCAATAGGATTA
CGCTGAGGCCCAGGCTAGGTGAGAATTTTGGAAGGGAGCGCTGGGATGCCAAGATCCCCGGCAG
GGCCACTAGCCACTGTATTTTATACTGAAGAACTTGAGGCCCCAAAGGGCATCTCAGGGGTCCT
AGCCCTGTCTCTGTGCCCCTCCGTGAGGCAGCCCAGGCTCCCTGTGTTGACTTGGTGTCCAGGC
TCTGCTTCCTGGGGATAGGGCATGTTGGCAGCTCCTTCCTGAGCCCACGTTCTCCCCTGTGTCT
TCTATTGAGCTCTTCTGCCCAGGTGGGGCCCATGCCCTTAAGCAGTATAGGTGAGTGTATCCTG
TTGCGGTCATGGGCCATAAATGAGGCAGTGTGGGGTCATCGAGGGTCAGCCTTCAGGCCACTAG
CGTTTTCTCCAGGTGCCCCCGGGGAGGGAACATTGCTCCAGTAGGGTCACTTCCTTGGGAATCT
GACCCTAGGAGCACGCATTTCCTGAAGCCCTGCAAAGGTGATGGTGGTCCAGGCAGGAAAGCGT
TCCTTCAGCCCAACATACAGCAGCTCCCAGCTGAAATTTGGTGGGCTCGTCAGGCCTTGAGGAC
TGCCCAGGCTTCATGATGGGGGACACAGAAGCTCTGGGGCTCCCTGGGGCTCACACTGGAGTTC
TCCCTCCCTGAAGAGTGGTGGGTGATTGGGCCCACAGTCAGGTGGCGGAGATTCAAGGAATGCC
CCCACCCCTGGCCCTGAATGGGACCAACTCTGTTGAGTAATGGGCCATCTTAATGGCGCCTCCT
TGAGCCTGTTGGGGGTGGGTTGCTGTTTCAATAAGGTTGAGTCAAAGCTGAGCTCTGACTGGGC
GCCTGGTGGTACTGCAGGCCGGCCCTTCCTTGGGCCAAGGGGGCAGCTTGTGTCCCGCTGGTGT
CATCCCTTCCTTGAGGTCCAGTGGTGGAAAGGGTAGCCTCCTATGGTGGGAAGGCCTGCTGCTC
AGGGACCTGTCCTGGAAATTTGCCATCCAGGCTGCTTCTGAGCCATGGGGTCACCTCCAGGGTA
GTGGGGTCTTGAGTCCAAGAAAAGGAGAGGACACCATGTGGTTCCTGATTGGCCAAGGCTCCTT
GGTTGGCCAGGAGCAGGTTGGTTGCCCTGGCCCATGCCCCATGGTCCTGGTGTGGCCCTGGGGT
TGTTCAGGGGAGCTGGCCCAGGCCGTGAAGGTGCTCTCAGGGCCTGCAGTAGAGTGCAGCCCAG
GCATCTTGCCCCAGCCCAGTGACCCACTTACCCTGTCTTTCTAAGGTTTGGTGGCCAGGAGGGC
CAATGGTGCCTTGTGCCCATGTTGGGTCAGTGTTCTGCATGGATCCCCATCATATGGGTATTCC
CTACCCACCCTCCCTGCACTGATATGTGAGGCCCTGGTTGGACACATGTCCTCCTCCTGTCCCT
TCAGATGGTGAGTCTGGAGGAAGACTTGTGTTGGACCCCCACAGGCCCCAGGCCAGTGTCCATC
AGCCTGGTGCTAATCCCCCAGTGCTCTGGAGTTTGGACCCTCCCTGGCACCCTGGTCTCCTGCA
CTGAGCTGTGGTGAGCACATGGATGCATGCTTGGGGTGGGGGTTGCCCTTGGTTGGGTCAATGA
TGAGAACCTTATATTGTCCTGAAGAGCGGTGATGACTTAAAAATCATGCTCAATAGGATTACGC
TGAGGCCCAGCCTAGGTGAGAATTTTGGAAGAGGACACTGGGCTCCCCGGAATGGCCACTGTAT
TTTGCGCTGGACACCTGCAGGCCCTGAGGGCCATCTGGAAGGTCCAACCCTGTCTCTCTGCTCC
TCCAGGAGGCAGCCCAGGCTCCCTGAGGGGGCTTGGTGTCGGGCTCCTGTTCCCCGGGGTTGGG
GCCGGTTGGTGGCTCCTTCCTGAGCCCCCATTCTCCCCTTGTGTCTTTCAGTGAGCTCTTCCAC
CCGGGCGGGCCCCCGGCATTGACTGGCATAGGTGAGTGGATCCTGCTTGTGTCATGGGCCATGA
ACCGGTCCATGTGGGGTCACCAAGGGTCAGCCTTTGGGATAAGAGGGTTTCCTCAGGGAACCAA
CCTGAATTCCCATCAGAGAGGTATGGGTTCTCCAGGAGGGCCGCTTTTTTGGGAATTGGACCCA
AGGAGGATAACTTCCCTGATCTCTGCAGGAGTGACGGTGGTCCAGGCAGGAAAGAGTTCCTTCA
GCCCCACCCAGCGCAGCGTTCTGTTGAGACTCAGTGCTCAGGTCAGGCCTGGAGGATTTCTCAG
GCCATCTGAGGGGGTTAGAGGGGAGCTCTTTGTCCATGGAGTGTGGTGCTTGAATCTGCCCTTG
GGGTTGGGTCATGGAGATTCAAGGATGGTGCCTCACCCATGGCCCTGATTGGAATTGTCCATTG
AGAAATACGCCATTCCAGTGGGCCTCCTGGAGCCTGATGGGGGGTGGTGTTTTTTTCCCTGGGG
TTGAGTCGTTGATGGGGACTCACCATAGAAAGCATGTGGCTCTGGAGGCCCCCAGGCTTTGATT
TCCAGTGCCTGCTTGGAGTGGGCACCAGGGCCGGCACCCACCATGCCCAAGGGCAGGCTCTGCG
ACTGCCAGGTCTGCATTGGCTCCTGGCATCAGGACCAGCCTCCTTGGTTGGCCATCAGCTGCTT
GGTTGCCCTGGCCCATGCCCCGTGGTCCTGGTGTGGCCCTGGGGTGGTTGAGTGGAGCTGGCCC
AGGCAGTGGAGGTGCTCTTGGGGCCTGCAGTAGGGTGCAGCCCAGGCATCTTGCCTGATCCCAG
TGACCTGCTTGCCCTGTCTTTCCAGGATTTTGTGGCAGGGAGGGCCAAGGGTGCCCTGCATCCG
TGATGGGTCAGTGTTCCACGTGGATCCCATTACGTGGGTCCATAGAGGATGAGCATGCGTGAGC
CAGCGCCCCCTTCCCACTTCTTCTCATGGAGCTGTGAGGCCCTGTGTGGACAGCTGTCTTCCTC
TTGTCCCACCAGGTGGTGAGCCCGGAGGAGGACTTGCGTTCGGCCCGCTGGTCCCAGTGTGTCC
GGAATTGGTGGGTTCTTGGTCTCACTGACTTCAAGAATGAAGCTGCGGACCCTCGCTGTGAGTG
TTACAGCTCTTAAGGTGGCATGTCTGGAGTTTGTTCCTTCTGATGTTCGGATGTGTTCGGAGTT
TCTTCCTTCTGGTGGGTTTGTGGTCTCGCTGGCTTCAGGAGTGAAGCTGCAGACCTTCGCGGTG
AGTGTTACAGCTCATAAAAGCAGTGTGGACCCAAAGAGTGAGCAGTAGCAAGATTTATTGCAAA
GAGTGAAAGAACAAAGCTTCCACAGTTTGGAAGGGGACCCAAGTGGGTTGCCACTGCTGGCTGG
GGCAGCCTGCTTTTATTCTCTTATCTGGCCCCACACACATCCTGCTGATTGGTAGAGCTGAGTG
ATCTGTTTTGACAGGGCGCTGATTGGTGCGTTTACAATCCCTGAGCTAGACACAAAGGTTCTCC
ATGTCCCCACTAGATTAGCTAGATACAGAGTGTTGACACAAAGGTTCTCCAAGTCCCCACCGGA
GTAGCTAGATACAGAGTGTCTATTGGTGCATTCACAAACCCTGAGCTTGACACAGAGTGCTGAT
TGGTGTGTTTGCAAACCTTGAGCCAGATACAGAGCGCGGATTGGTGTATTTACAGTCCCTGAGC
TAGACATAAAGGTTCTCCAAGGCCCCACCAGAGAAGCTAGATACAGAGTGTCGATTGGTGCATT
CACAAACCCTGAGCTAGACACAGGGTGCTGATTGGTGTGTTTACAAACCTTGAGCTAGATACAG
AGTGCCGATTGGTGTATTTACAATCCCTGAGCTAGACATAAAGGTTCTCCAAGGCCCCACCAGA
CTCAGGAGCCCAGCTGTCTTCACCCAGTGGATCCCGCACTGGGGCTGCAGGTGGAGCTGCCTGC
CAGTCCTGCGCCATGCGCCCGCACTCCTCAGCCCTTGTGTGGTTGATGGGACTGGGCACCGTGG
AGCAGGGGGCGGCACTCGTCGGGGAGGCTCTGGCTGCACAGGAGCCCATGGAGGGGGTGGGAGG
CTCAGGCATGGCAGGCTGCAGGTCCCGAGCCCTGCCCCGAGGGAAGGCAGCTAAGGCCCGGTGA
GAAATCAAGTGCAGCGCCGGTGGGCTGGCACTGCTGGGGGACCCAGTATACCCTCTGCAGCTGC
TGGCCCGGGTGCTAAGCCCCTCATTGCCTGGGGCCGGCAGGGCCGGCCCGCTGCTCCGAGTGTG
GGGCCCGCCAAGCCCACACCCACCCGGAACTCCAGCTGGCCCACAAGCGCCCCGCGCAGCCCCG
GTTCCCACTTGCGCCTCTCCCTCCACACCTCCCTGCAAGCTGAGGGAGCAGGCTCCGGCCTTGG
CCAGCCCAGAAAGGGGCTCCCACAGTGCAGCAGTGGGCTGAAGGGCTCCTCAAGTGCCACCAAA
GTGGGAGCCCAGGCAGAGGAGGCGCCAAGAGCAAGTGAGGGCTGTGAGGACTGCCAGCACGCTG
TCACCTTTCATCAGGACAGTGTCTGTCAGCCAGGTGCACAGCCCTTGGTGTGCTGGAGCTTGGG
CTTTTCTTGGCAGCCTGGTGTCCTGCATTGAGCGGTGGTGAGCTTGTCCAGGTCCCGCTGGTTA
CCTGCACGGTGACGGGGGTGCCCTCGGTTAGGTTGATGATGAGAACCTTATTTTGTCTCGAGGA
GAGGTTATGTGATGGGTGTTGTGGCAGCTCTTTGTATTTCATTTTCTTCAGTGCTTTTTGTGTG
TTCTCTTTGTATTTTTTACCGCTTTGTGTATTTTACTTTCACAAAGGCAGCAGCAAATGTTTTA
TTGGTGGTGGTATTTTTCATTTTTATTTACTTCCTAATTTGATAGTGTTTTAGTTGGAATACAC
AGTCTCATTGTAGTTGTTCAGAAATATGCTTTTTTTCTGTACATTATATGATTGTGCATAATTT
TTGCCAGTTTTTTTTTTAATTGGGGACTACAGTTGAATGCACTGCCTATCTCCTACTTTACTGA
AACAGATTGTATGGAAATCTAAAGACGAGAAAGTAGACAAGCACAGCAGAGGACATTTATTTTG
ATATAACCACAGAAATTCATTGATATGACCTTATCATATTGATGGTTTTTCAAAGAAATATAAA
ATCTTTCGATAATGGATTGTCTCAGTTGCGTTTCTCCTTATATCATGTTTTGCCTTCAATACAA
ATTGCAATGCTTAGAAATTTAAAGAACACCTTCTGAAGTGGAAGATCTAGCTAAAGAATTAGAT
TGGTATTGGCTGAAATGTTGCAGTAATCACATGATAGGGTTAATACAACATGAGTTACATGCTG
AGTGCTCTAAGGTAGAAAGTAGGTAACAAGGAATAACAGGTAGGTAATGTAAGTCGGGAGAAGG
AAACTCTAGGACAGAATCATTAGAAAAGGCTTGACGTCAACAGCAGTGTATAGAATTTACAGTG
TATAGAATTTACAGTGCATAGAATTTACAATAAGTGCCTTTTTTTGTGCTCATCAATGCTCTTG
CTTATATTGTAGGAGGAGTTAGTTCCCTTGAAGAAATATCAATACAAATTTCCAAATTGCAATG
CAGAGGGAAAAAAGAATGAGAAAGTAAAAAATATCCAAGAACTCTGGAAGCATTTCAAAAAGTG
TAAAAATGCATAGTGACAATGTCAGGAGAATAAGGAGATAAAGGAAAAGAAATATTTGACACAA
CAATGACTTGAGCACTTTCTAAAATTAATGATGGACAGAAACCCACATGTTCAGGAAGCTCAGA
GAGCACTCTGCAGAATAAAAATATTTTACAAATCGACACCTTTGCATTTCATATTTAAAGTCCA
GAAAATCATAGACTAAGGGAAAAGCTTGATAGGAGCAATAGGAAGAAAACCATCTTACCTATGG
AGCAACAAGAATAAAAATTGCTTTGAAATTATCTTCAGAATCCATGAAAGCAAGAAGTGAGTCA
GGGTGAAATATTTAGTGTTAAAAGTAAGAAACAAAAAAAAGTACCAACTTTGAATTTTCTGCTC
ATCAAAATTATTCTTCAAAAGTGAAGGAGAAATACTCTCTGAGACCAACAAAAATTGCTAGAAC
TTCTTGCCACTACACCAACCTTTTAATAAACGTAACAAGAAATTCTTCAGAAAGGATAAAAATG
ACATAGGTCGGAAAATCTAATTTACATAAAGAAATGAAAAGCATTGGAGAAGAAATATATGAAG
AAAAATTAAATATATATGTATTTTTTGATTCATAATTGATCTTTCAACAATTTGTTCAAAATAA
TAACAAGATTATATTTGGTGATTATAGGTTATGGAAAAGTGAAATGAATGACAGCAATATTATA
AGTCATCCGGTTCCAAGATGGCCGAATAGGAACAGCTCCAGTTTACAGCTCCCAGTGTGAGTGA
CGCAGAAGACAGTTGATTTCTGCATTTCCAACTGACGTACCAGGTTCATCTCACTGGGGCTTGT
TGGACAGTGGGTGCAGCCCATGGAGTGTAAGCCGAAGCAGGACGAGGCATCACCTCACCTGGGA
AGTGCAAGAGGTCAGGGAATTCCCTTTCCTAGCCAAGGGAAGCGTGACAGATGGTACCTGGAAA
ATTGGGACACTCCCACCCTAATACTGTGCTTTTCCAACTGTCTTAGCAAACGGCACACCAGGAG
ATTATATCCCGTGCCTGGCTTGGAGGGTCCCACATCCACGGAGCCTTGCTCACTGCTAGCACAG
TAGTCTGAGATCAAACTGCAAGGCAGCAGTGAGGCTGGGGGAGGGGCATCCACCATTGCTGAGG
CTTGAGTAGGTAAACAAAGCGGCTGGGAAGCTCGAACTGGGTGGAGCCCACCACAGCTCAAGGA
GGCCTGCCTGCTTCCGTAGACTCCACCTCTAGGGGCAGGGCATAGCAGAACAAAAGGCAACAGA
AACTTCTGCAGACTTAAACATCCCTGACAGCTTTGAAGAGAGTAGTGGTTCTCCCAGTACAGAG
TTTCAGATCTGAGAACAGACAGGCTGCCTCCTTAAATGGGTCCCTGACCCCCAAGTAGCCTAAC
TGAGAGACACCTCCCAGTAGGGGCTGACTGATACCTCATACAGCTGGGTGCCCCTCTGAGACGA
AGCTTCCAGAGGAAGGATCAGGCAGCAACATTTGCCGTTCTGCAATATTTGCTGTTTTGCAGCC
TCCACTGGTGATACCCAAGCAAACAGGGTCTGGAGTGGACTTCCAGCAAACTCCAACAGACCTG
CAGCTGAGGATCCTGACTGTTAGAAGGAAAACTAACAAAGAGAAAGGACATCCACACCAAAACC
CCATCTGTACGTCACCATCATCAAAGACCAAAGGTAGATGAAACCACAAAGATGGGGAGAAACC
AGAGCAGAAAAACTGAAAATTCTAAAAATCAGAGCACCTCTTCTCCTCCAAAGGAATGCAGCTC
CCTGCCAGCAATGGAACAAAGCTGGACGGAGAATGACTTTGACGAGCTGACAGAAGTAGGCTTC
AGATGATCGCTAATAACAAACTTCTCTGAGCTAAAGGATGAGGTTCAAACCCATCGCAAAGAAG
CTAAAAACCTTGAAAAAAGATTAGATGAGTGGCTAACTAGAATAAACAGCATAGAGAAGACCTT
AAATGACCTGATGGAGCTGAAACCATGGCACAAGAACTACGTCACACATGCACAAGCTTCAGTA
GCCGATTTGATCAAGTGGAAGAAAGGGTATCAGTGATTCAAGATCAAATGAATGAAATGAAGCA
AGAAGAGAAGTTTAGAGAAAAAAGAGTAAAAAGAAACAAAGCCTAAAAAAGTAAAAAAGTAAAA
CAAAGTAAAAAAGAAACATGGGACTATGTGAAAACACCAAATCTACGTCTGATTGGTGTACTTG
AAAGTGATGGGGAGAATGGAACCAAGTTGGAAAACACTCTGCAGGATATTATCCAGGAGAACTC
CCCCAACCTAGGAAGGCAAGCCAACATTCAAGTTCAGAAAATACAGAGAATGCCACAAAGATAC
TCCTTGAGAAGCGTAACTCCAAGACACATAATTGACAGATACACCAAAGTTGAAATGAAGGAAA
AAATATTAAGGGAAGCCAGAGAGAAAGGTCAAGTTACCCACAAAGGGAAGCCCATCAAACTAAC
AGCAAATCTCTCAGCAGAAACTCTACAAGCCAGAAGAGAGTGGGGGCCAATATTCAACATTCTT
AAATGAAAGAATTTTCAACCCAGAATTTCATATCCAGCCAAACTAAGCTTCATAATTGAAGGAG
AAATAAAATACTTTACAGACAAGTAAATGCTGAGAGATTTTGTCACCACCAGGCCTGCCCTACA
AGAGCTCCTGAAGGAAGCACTAAGCATGGAAAGGAACAACCAGTACCAGCCACTGCAAAAACAT
GCCAAATTGTAAAGATCACTGATGCTAGGAAGAAACTGCGTCAACTAACGAGCAAAATAACCAG
CTAACATCACAATGACAGGATCACATTCACACATAACAATATTAACCTTAAATGTAAATGGGCT
AAATTCTCCAATTAAAAGACACAGACTGGCAAATTGGATAAAGAGTCAAGACCCATCAGTGTGC
TGTATTCAGGAGACCCATCTCATGTGCAGAGACACACATAGGCTCCAAATAAAGGGATGGAGGA
AGATCTACCAAGCAAATGGAAAACAAAAAAAAGCAGGGGTTGCAATCCTAGTCTCTGATGAAAC
AGACTTTAAACCAACAAAGATCAAAAGAGACAAAGAAGGCCATTATGTAATAGTAAAGGGGTCA
ATTCAACAACAAGAGCTAACTATCCTAAATATACATGCACCCAATACAGGAGCACCCAGATTCA
TAAAGTAAGTCCTTACAGACCTACAAAGAGACTTAGACTCCCACACAATAATAATGGGAGACTT
TAACACCCCACTGTCAACATTAGACAGATCAACAAGACAGAAAGTTAACAAGGATATCCAGGAA
TTGAACTCAGCTCTACACCAAGCAGACCTAATAGACATCTACAGAACTCTCCACCCCAAATCAA
CAGAATGTACATTCTTCTCAGCACCACATCGCACTTATTCCAAAATTGACCACATAGTTGGAAG
TAAAGCACTCCTCAGCGAATGTAAAAGAATAGAAATTATAACAAACTGTCTCTCAGACCACAGT
GCAATCAAACTAGAACTCAGGATTAAGAAACTCACTCAAAACCACTCAACTACATGGAAACTGA
AAAATCTGCTCCTGAATGACTACTGGGTACATAATGAAATGAAGGCAGAAATAAAGATGTTCTT
TGAAACCAATGAGAACAAAGACACAACATACCAGAATCTCTGGGACATATTTAAAGCAGTGTGT
AGAGAGAAATTTATAGCACTAAATGCCCACAAGAGAAAGCAGGAAAGATCTAAAATTGACACCC
TAACATCACAATTAAAAGAACTAGGGAAGCAAGAGCCAACATTTTCAAAAGCTAGCAGAAGGCA
GGAAATAACTAAGATTGGAGCAGAACTGAAGGAGATAGAGACACAAAAAACCCTTCAAAAAATC
AATGAATCCAGGAACTGGTTTTTTGAAAAGATAAACAAAATTGATAGACCACTAGCAAGACTAA
TAAAGAAGAAAAGAGAGAAGAATCCAATAGATGCAATAAAAAATGATAAAGGGGATATCACCAC
CAATCCCACAGAAATACAAACTACCATCAGAGAATACTATAAACATCTCTATGCAAATAAACTA
GAAAATCTAGAAGAAATGGATAAATTCCTGGACACATACACCCTCCCAAGACTAAACCAGGAAG
AAGGTGAATCTCTGAATAGACCAATAACAGGCTCTGAAATTGAGGCAATAATTAATAGCCTACC
AACCAAAGAAAGTCCAGGACCAGAGGGATTCACAGCTGAATTCTGCCAGAGGTACAAAGAGGAG
CTGGTACCATGCCTTCCGAAACTATTCCAGTCAGTAGAAAAATAGGGAATCCTCTCTAACTCAT
TTTATAAGGCCAGCATCATCCTGATACCAAAGCCTGGCAGAGACACAACAAAAAAGAGAATTTT
AGACCAATATCCCTGATGAACATCGATGCAAAAATCTTCAATAAAATACTGGCAAACCGAATCC
AGCAACACATCAAAAGCTAATCCACCACGATCAAGTTGGCTTCATCCCTGGGATGCAAGGCTGG
TTCAACATACACAAATCAATAAACATAATCCATCATATAAACAGAACCAAAGACAAAAACCACA
TGATTATCTCAACAGATGCAGAAAAGGCCTCGACAAAATTCAACAGCGCTTCATGCTAAAAACT
CTCACTAAACTAGGTGTTGATGGGACGTATCTCAAAATAATAAGAGCTGTTTATGACAAACCCA
CAGCCAATGTCATACTGAATGGGCAAAAACTGGAAGCATTCCCTTTGAAAACTGGCACAAGACA
GGAATGCCCTCTCTCACCGCTCCTATTCAACTTAGTGTTGGAAGTTCTGGCCAGGGCAATCAGG
CAGGAGAAAGAAATAAAGGGTATTCAATTAGGAAAAGAGGAAGTCAAATTGTCCCTGTTTGCAG
ATGACATGATTGTATATTTAGAAAACCCCATCGTCTCAGCCCAAAATCTCCTTAAGCTGATAAG
CAACTTCAGCAAAGTCTCAGAATACAAAATCAATGTGCAAAAATCACAAGCATTCCTATACACC
AATAACAGACAAACAGAGAGTGAAATCATGAGTGAACTCCCATTCACAATTGCTTCAAAGAATA
AAATACCTAGGAATCCAACTTACAAGGGATGTGAAGGACCTCTTCAAGGAGAACTACAAACCAC
TGCTCAACGAAATAAAAGAGGACACAAACAAATGGAAGAACATTCCATGCTCATGGATAGGAAG
AATCAGTATCGTGAAAATGGCCATACTGCCCAAGGTAATTTATAGATTCAATGCCATCCCCATC
AAGCTACCAATGACTTTCTTCACAGAATTGGAAAAAACTACTTTAAAGTTCATATGGAACCGAA
AAAGAGCCTGCATTGCCAAGTCAATCCTAAGCCAAAAGGACAAAGCTGGAGGCATCACACTACC
TGACTTCAAACTATACTACGAGGCTACAGTAACCAAAACAGCATGGTACTGGTACCAAAACAGA
GATATAGACCAGTGGAACAGAACAGACCCCTCAGAAATAATACCACATGTCTACAACCATCTGA
TCTTTGACAAACCTGACAAAAATAAGAAATGGGGAAAGGATTCCCTATTTAATAAATGGTGCTG
AGAAAACTGGCTAGCCATATGTAAAAAGCTGAAACTGGATCCCTTCCTTACACCTTATACAAAA
ATTAATTCAAGATGGATTAAAGACCTAAATGTTAGACCTAAAACCATAAAAACCCTAGAAGAAA
ACCTAGGCAATACCATTCAGGACATAGGCATGGGCAAGGACTTCATGACTAAAACACCAAAAGC
AATGGCAACAAAAGCCAAAATAAACAAATGGGATCTAATTAAACTAAAGAGTTTCTACACAGCA
AAAGAAACTACCATCAGAGTGAACAGGCAACCTACAGAATGGGAGAAAATTTTTACAATCTACT
CATCTGACAAAGGGCTGATATCCAGAATCTAGAAAGAAGTTAAACAAATTTACAAGAAAAAAAT
CAACCCCATCAACAAGTGGGCAAAGGATATGAACAGACACTTCTCAAAAGAAGACATTTATGCA
GCCAAAGACACATGAAAAAATGCTCATCATCACTGGCCATCAGAGAAATGCGAATCAAAGCCAC
AATGAGATCCCATCTCACACCAGTTAGAATGGCAATCATTAAAAAGTCAGGAAACAACAGGTGC
TGGACAGGATGTGGAGAAATAGGAATACTTTTACACTGTTGGTGGGATGGTAAACTAGTTCCAC
CATTGTAGAAGACAGTGTGGCAATTCCTCAAGGATCTAGAACTAGAAATACCATTTGTCCCAGC
CATCCCATTACTGGGTACATACCCAAAGGATTATATATCATGCTACTATAAAGACACATGCACA
TGTATGTTCATTGTGGCACTATTCACAGTAGCAAAGACTTGGAACCAACTCAAATGTCCATCAA
CGATAGACTGGATTAAGAAAATGTGGCACATATACACCATGGAATACTTTGCAGCCATAAAAAT
GGATGAGTTCAAGTCTTTTGTAGGGACATGGATGAAGCTGGAAACCATCATTCTCAGCAAACTA
TCGCAAGGACAAAAAACCAAACACTGCATGTTCTCACTCATAGGTGGGAATTGAACGGTGAGAA
CACTTGGACACAGGGTGGGGAACATCACACACGAGTGGCTGCCGTGGGGTGGGGGGAGGGGGGA
GGGATAGCATTAGGAGATATACCTAATGTAAATGACGAGTTAACGGGTGCAGCACACCAACATG
GCACATGTATACATATGTAACAAACCTGCACGTTTTGCACATGTACCCTAGAACTTAAAGTATG
AAATATATATATATATATATATATATAGTCATGAGGGAGAGAAAATACTCTTAGTTACTTGGAC
TAACCCACGAAGTAGGACAGTATAATTTGAAAAAGGACTTGGATTAGTTGTAAACGTATAATGT
AATATCCAGCACAGCCACTAAAAATGATTTTGAAAAAAGCAGTATAATCAATATACTAACAGAA
GAAAAAGCAGAATCAGGCCAGACATGGTGACTCATGTCTGTAATCCCAGCACTTTGGGATGCTG
AGGCAGGAAGATCACTTGAGCCCCGGAGTTCAAGTCCAGCCTGGCAACGAATAAGAGCCCATCT
CTACCCTCCACCCCCCCCAAAAAAAAAAAGAAAAAGAAAAAGAGGCCGGGCACGGTGGCTTACT
CCTGTAATCCCAGCACTTTGGGAGGCCGAGGTGGGTGGATCGCGAGGTCAGGAGATCAAGACCA
TCCTGCCTAACACGGTGAAACCCCGTCTCTACTAAAAATACAAAAAAAAAAAAAAAATAGCTGG
GCATGGTGGCGGGCGCCTGTAGTCCCAGCTACTCAGGAGGCTGAGGCAGGAGAATGGTGTGAAC
CTGGGAGGCAGAGCTTGCAGTGAGCCGAGATTGCGCCACTGCACTCCAGCCTGGGAGACAGCCG
CAAGATTCTGTCTCAAAAAAACAACAACAACAACAAAAAAAAACAAAAACAAAAACAAAGAAAA
CGAAAAGAAAAAGCAGAATCTTTTTTTTTTTTTTTTTTTTTTTTTTTTTGAGATGGAGTTTTGG
TCTTGTTGCCCGGGCTGGAGTGCAATGGCACGATTTCAGCTCACCACAACCTCCACCTCCCAGG
TTCAAGCAATTCTCCGGGCTCAGCCTCCCGAATAGCTGGGATTACAGGTGTGCACCACCATGCC
TGGCTAATTTTTTGTATGAAAAAGCAGAATCTTATACAATGCTCAATTAAAATCAGGGAGAGGA
GAAAAAGAGTAGAATACCAAAAAAGTAACAAATTACAAGGTTGATATAAATAGAAAACAATCAC
AAATACTGAGTATACTAATCTAAGTATATCAATAATCACTTTAAACATCGCTTAAAAGACAGAG
ATTGTCAGTGGATTAAGAACAAGACCCAAATATATGTTGTCTATGAGAAATGCACTTTAAATAG
CTGGGCATGGTGGTGTGCACCTGCAGTCCTTAGGAGGCCAAGGCGGAGTGTCACCTGAGCCCAG
GATTTTGGGACTACAGTGAGCTATGATCATGCCACTGCACTCTAGCCTGGTTGACAGAATAAGA
CCTCATCTCTATAGAAAAAAGTAAGTAAATCTACTTTATAAAGGCACAAATATATTCACAGTAA
AGGAATAGAGAAAGATATCCCATGCTAACATTAATCAAAATAAAGTGAAATAGCCATATTAATT
TCAGACACAACCGACTTTAGAACAAGGAATATTATCAGAGATCAAAAGGGACATTCCTTAATGA
TAAAGGGATCAGTTTTCTAAGAAGACATAACAATCCTTAATGTGTACGCACTGAACAGCAGATC
ATCAAAGTACATGAAGCTAAATCTGATAGAACTGCATAGAGAAATAGATAAATTCACTCTTAGA
GTTGGAGGCATTGGCATCCCTCTATCAGAAATGGACAGGTTTAGCAAGCAGAAAATCAGTAAGG
GCATAACATGATCAATCATGATCAAAAACTTGATTCAGTCAACTATATGTAACTGACATCTATG
GACCACTTTACCCAACAACAGCAGATGACACATTTTCTCACACACACATGAAACATTCAGCAGA
ATAGACCACATTCTGGGCTAGAAAATGCACCTCAACAAATTTAAAAGAATAGAAATCATACAAA
GTATGTTCTCAGACTACAATGGAATGAAAGTAAACAAAAATCACTAAAAAAGATAGCTGAAAAA
CATGAAAATACTTGGAGGTTAAACAACAGACTGAAATAACTGAAGGTCACAGAAGTCTCAAGAT
AAATTTTTAAAATATTTCAAACTAAATGGAATTGAAAGCACAACTTATCAAAAATTTAGGAACG
CAACAAAGGCAGTGCCTACTGGGAAATTTATACCACTGAATGTATGTTAAAAAGAACAAAGATC
TAAATAAATAATCCTTCCCATTGGAAAGCTAGGGGGAAAATTCAAATTAAATTCAAAGTAAGCA
AGTAAAAGAAATAATAAAATATGAATACAATTCTGAGTGGCCTGATATCTGATTTCTGGAAGCT
GAGTTTTATGTGGCTTTAGAAGTTTCATGTTATGAGTGTGAAATGGCAAGTGACCATGGCGATT
GCTTCTGATATGGCCACCTCAATTACATAGAGAAGGGATGATCAGTGGCATCATCCCAGTTCTG
CATGATACCACACCAGTGATTCTCCAAAGTCAAGAAACCTGTATATGTTGTCCTTGTGTTTGGT
GAATCTGTGGTCTATAGACTACATAATGGGCTATTTCTGGGGTACTGACCATTTTGCTCCATTA
AGGAAAACCATGCATGAGCCATATAACTATGGGAAACAATGTTTTCCTTTTTGACACCTGGCCT
ACTCTCTTCTTAGGTGAGAGCAAAGGAAGAGCACCACTTGGCCTGCTGCACCCAGGGCAATTTA
TACCATCCAGATATCCAGCACTGAATATGTAGCAACTCCAATCCACCATAGAACATTAATCTAT
CTCTGATAAGCATGCTAATATCTATGATTCTGCCTTTGTGGGGAGATGACTCCATAGATTAACC
CCCTGGCTTGGGGCAATATGGAGATGTTATATTGTCTTCGACAGGGAAGATGACATAAAAATTA
TGTTCAATAGGATTATGTGGAGACTTAACATAAGTGACAAACCTAAGACAAGAAGTTAGAAATC
CTTGTTTCTTTTAGGTGAATAATTTTGGAGCTTGAGAAAAAGACACCATTGTGGCGAACTTGCT
TTGCCAAACCCTGTGTATGCATTTTTGGGTAAACAACACATTTTCATCCTTTAAGCATTGTTTC
CATTATTTATAATTCCTAATAGTATGAGACATTTAGGACTCATTCCTTGGTTCTTCTTCTCTCA
TTGTATCTTTCATTGTATCTTTCAGTGCTGCAAGAGATGCAATCATCCCATTAATGAAAGCAGA
GTGACTGCCTTCGTCACACATACATTCAAAATAGATCACTAGGCTAGTGATGATTCTCTCTTAA
GGCTATTATGTTTACTCATTTAAAAAATCTGGATCTCAGTATAGATTTTCTTCTCAGTGAGTGA
CTGACCTATCTGATATGATGCTATGACAAAATCTTTACTTTTTACCTGAATTTGGTGGGATTGA
AAGTTGAGAACATATTAATTTATTTTCTGTTTCACTTGATTAATAATGCCCAATCTAACATGCA
GTGTTCACTTGGGTACATATATACAACTACATCAAATTTTTTCTTGTTTTACCTTAAAAGTGTT
AAGATATTGTAATGGTATGGCCAGTTTACTCACTGGTAAACTTTTCATAACAGAAATATCATGC
CAGTGGACACAAATATGTAACATCAGTAATTTGCTTTCCTCATGAAACAGAGAACTGAATAAGT
TGATGGGTAAACTAGTATCTGAATTATAAACCCCAAATATCATGTGACTTCATCATTTTCAGTA
TAATGAAAATACAGGCTGTGAATGCTTCCAGTTGGTCCTGAAAGGGCCTTTGAGGTGAGTAGGT
TCACCAAAGCGGCAACATATGTGTCCCAGTGGGCATTCTACTCCTCAGTAAGTGACTTGACATT
CTTTATGGCTTCCTCATGATTAAAACAAGTCTTTTTATTGAAATTTTACTGAAATAATGATGAA
ACAATGTAATATTCTTTTCATCTACATATAAGAGTAGACATAGATTAACCTCTACCTTTTTTTT
TTTTTTTTTTGAGACGGAGTCTCGCTCTGTTGCCCAGGCTAGAGTACAGTGGTGCGATCTCAGC
TCATTGCAACCTCTGCCTCCCAGGTTCAAGCGATTCTCCTGCCTCAGCCTCCTGAGTAGCTGGG
ATTACAGGCACGTGCTACCATTCCTGGCTAATTTTTTTGTAATTTTAGTAGAGACATGGTTTCA
CCATGTTGGTCAGGCTGTTCTTGAACTCCTGACCTTGTGATCTGCCCACCTCTGCCTCCCAAAG
TGCTGGGATTACAGGCTTGAGCCCCCGTGCCTGGCCTTCACCTCTACCTATTATAATATTTTCA
TGTTATTCCATTTTGTCATGTTTGTGGGTCATTTGATGAGGGTTCACAATATGAAAACTGGGCA
TTTTGGCCAAGATGCATGTAAATGAATACAACTCCATGTGCCCTGATAACTGATTTCTAGATCC
TGAGATGCAAGTGGCTTCCAGAGTGTCTGTATTTTATGAAAGTTCATATACAGTAAACCATGCT
AATTGTTACTGATACAGCTTCCTTGAATATATAAAGCGATGATTAATGGGATGTTTTGTTTCTG
CAGAAAACCACTGAATTGCTTCTCCAAAGCCAAGTGACCTGTATGATTTGTCCTTATTATATTT
TGTGAACCAGTGGACTAAAACCTTTATAATGGGTCATTTTTTTAGTATTGTCAATATTGCTCAA
TTAACAAACATGACTCATATACACAGGTGAGATGTAGTTCTCCCTTTGACATTTGCTCTACATC
TGTCCCTTAGATGATGATATGGAAGAAAAGCACTCTTTGGCCTGTTGTGACTGGGACAGTTGAC
AGCACCCAGGTGTCCTTTAATGAAAATGCTCTTGACACCAATGCATCCTAGCATCACAGCTTCA
GGAAGCCTTCTCAAGTGTGCATGGGGAGTACTATGTCTTTCATCAATAATGAAATCTTCTGATT
TGGTGAGAAATAATGCCTTAAAATTACACTCAATAGGATTATGCTGAGGCTCAGCCTACCTAGA
TGAAAATGCTGAAACAGATAATAGTGACTTCTTATTTCCCTTCAAGGGGAATTTTATTTTGAAC
TTGAAATAACAAAATCTTTATGGGTAACTTGTTTGCCCAACTGTATCAGTGCACCTATAAATGA
ACAAAAAATTCTCAGCCTATAAGCTTTGCTTCAGTGACCTTTAATTTCTAATAGTAAGGTATAT
TCAGGTCTCATTCTTTGGCTTTTTCTCTTATTGTGTATTTCAGTAAGACATGCTGCCAAGAGAT
GTGCCATTCTATTATAAAAGATCAGTAGCTTCCTTTACCGACGTGTATATTCTATCTAGAACAT
TGAGCTATGGAAGACTCCCACCTAAGGGAATTAGTTTTACACCTTCAGGTAATCTGAACTTCAA
TATGAGTTTTGCTTTATGATATAATCCTTATGACAAAATCTTTACTTTTTATCTGAACTTCATA
CAATCAATATTGAAGATTTTTTAAATTCACTGTTACCTAGTCCAATATTTAATATACATTTGGC
TGTAGATACATGACTAGACAAAAATTTTTTATATTATTATTGTGGGGGCTCTAACAGTGTTTCA
CTGTTTTGAGTTACTGATGAACTTTCTGTAACTGTGAGACCATGGTAATGGAAGAGACCGTGGT
AATGGGGAGGAGAATGTGAGGATCAGTATGTACTTTGCCTATAAAACTTAAATATTTAGTAATT
TGATCACCAAATTGATACTCAAATTTCAGTTCCTATCATGTGTAAACCAAAAAGTATCTGAGAT
TTAGAAGTTTATTTAGAAGTGGATTTTGCCAAGGTTAAGCACATGCTCAGAAGAAATAAACCCA
AAATCACAGAAACCGTCTGGGGTCTATGTCTTTCTCCAAAGATGATTTTGAGGGCTTCAGTGTT
TAAAGGGGAAAAGTGGGCTGGAGGGGGAAAAGGGAGGGTATGATAATCCGTATGTTCCAAGACA
AAAGGAGCAGGTAGGGGTATAGTCAATAATGTATTCATCTCATGCTCAGTAAATCAGCACTTTA
CATAAGATAAGGTGAACATAGAGAAGCTACCTGTGGAGATCTTTAACCTTTAATCTGTAGCTAT
CTGCTTAAGATCAAAAGGAAATTCTTGCATGACTCAGCTTTCAGCTTATTTTTTTTTTCTCTTG
GCGTAGTAAATTGGGGTCCCAAGTTTATTTTCCTTTCACACATGTGACCCCACTATTTTCAGAA
TAATGAAAGCACATGTTAGAAATGATGACACTTATTCCTAATATGGACTTTGAAATGACTGGGT
TCACAAATTTAGAATCATGGATCTCATTATAGGTTTTTATTTTCAATGAGTGACTGGCCTACTT
GATGGAATTCTTTCAAAAAATGTTCACTTTTTACTGAGATTATATTAAAATAATGTTGGAATGT
GTTCAGTCATGTTTACCATCTCTATAGTATGATATATGATAATATCGCAGATTACTCAGTGTCA
AAAACCTGGCATACCTTGTCCTTATGATGTTGATGAGTCAATGGTCTAGAGCCTTAGTAATATG
TCTTTTTGTGGTAGTACGGACCGTATCTGTCCACTAAGAAATAGCCTGTGTAAGCCATATACAC
ATGAACAATTTATGTCTGATTTTGAACTATGGTCCCATTCCTATGCCTGTAGATAAAGACTGCT
GAGAAGAGCACCCTCTGGTGTTGTCACAGAGGCAAGTGCTACCGCACAGGCATGCTGCAGTGAA
TTTAACTGATCCTCTGTCCCTGCAACCGTTGTTTAAGGATGCTATTCTGGTAAGGGTTTACAAG
ACAACGTAAATATATGTATAAAGAGTATCTTCAGGAGACGTAATAATGTAAAAATCATGCTCAA
TAGAATTAAGCTGAGGCTCAAAATAGTGCCAATCCTTGCAAGAAGGTGTGAGAAGTCATTAGAT
TTGGGGGGTATGGGTTATTCCATTTTGAACTTGGTAGGAAGAAACCCTTGGGGGAATCTTACAT
CATTTGCCTCTACCTACTTCTAAAGGAACTTTTAAAATTCTTTTATCTTGTCTTTATTCTTAAT
ACTCATACATTATTAGAGAGAATATCTTGGTTTATTTTTTCCTCATGGTGTGCTTCAGAATGTT
CATCCAGCAAGAGGTGTGTCATTACACGGATTATATATATGAGTTCTGTCTTTCGTAATTAAAA
AATTTCAAGTGGACCATTIGAATATTAAACATTGGCCTTCAAAATGATTACATTTACCTGTTCA
TACAAAATTAGTGAGCAATGATCTATTCTTTCCAATGAGTGACTGATCTAATGAGGTCCTCATT
AAAACTCCTTTTTTTTTTTTTTTTTTTTTTGCCTGAACGTACTTTGATTTAAAGATGTGACATT
TAGTAATTCATTGCAATCAACTCTTTAGTGATTGCACAAATAGACAATCCTTTTCTATTATTTT
ATTTTTTGGTTGCTGTGTATGGTGTTATGATGTTTGTGGATCATTGAGGCACTACTCACAACTG
AAAGACTTTGGCAATTTTAGGGAATGGCTTGAGATTCAGCTATAGGTTACCCTTGATTGAAATT
GAATACAATACAGGTAGTCTGAAATCTCATTTCCTTATCCTGAGTCTCACTCAGGTGTCATTAG
ATTCATTATAAAGAAATGCATACACTATGTATGATGCCAGTTGTTACTGATATGAAGACGATAA
GAAGCTTTTATTTCTGCAGGGAAATAATCCCTTACCAGGTCATCAAAAGACATGATACCTATGT
GCTTGGCCCTTACTGTACACGGGGAATCAGTGGTCTACCACAGCTTAAGTAACGGGTCATATTT
GGAGTATCACACATCTCAGTCTTGTAGAAATTAGGAACAGCAATTAGGAGTCATGCACATATAA
GAGATGTAATCCCACCCTTTGACTATAGCCTACTCTTGTCTTTTACAGAAAAGACTGTGGAGGA
AGAAAACCCTTTACCCTGTTGTTCAGGGAGAAACTGACACCACTCAACTGCCTGGCACTGAAAA
TGTGGCATCCAGTCCACTTTACCATCAGTGTTTAAGGAAACCATCTCTGGTAAGCATATTTGAT
CCAAGTGTGCATATGCACATGAGTGCCATGGGCTGGGTCAATGATGAGATGTTACCTTGAAGAG
AAATGATGACGTAAAAATTAAGTTCAGTTGGATTACGCTGAGGCCC
SEQ ID NO: 3
shRNA artificial/synthetic sequence.
5′-TGATGATGAGAACCTTATATTCTCGAGAATATAAGGTTCTCATCATCA-3′
SEQ ID NO: 4
SNORD115-1 human genomic sequence
GTGTTGATGATGAGAACCTTATATTATCCTGAAGAGAGGTGATGACTTAAAAATCATGCTCAAT
AGGATTACGCTGAGGCCC
SEQ ID NO: 5
SNORD115-5 human genomic sequence
GGATCGATGATGAGAACCTTATATTGTCCTGAAGAGAGGTGATGACTTAAAAATCATGCTCAAT
AGGATTACGCTGAGGCCC
SEQ ID NO: 6
SNORD115-9 human genomic sequence
GGGTCGATGATGAGAACCTTATATTGTCCTGAAGAGAGGTGATGACTTAAAAATCATGCTCAAT
AGGATTACGCTGAGGCCC
SEQ ID NO: 7
SNORD115-10 human genomic sequence
GGGTCGATGATGAGAACCTTATATTGTCTGAAGAGAGGTGATGACTTAAAAATCATGCTCAATA
GGATTACGCTGAGGCCC
SEQ ID NO: 8
SNORD115-12 human genomic sequence
GGGTCGATGATGAGAACCTTATATTGTCCTGAAGAGAGGTGATGACTTAAAAATCATGCTCAAT
AGGATTACGCTGAGGCCC
SEQ ID NO: 9
SNORD115-13 human genomic sequence
GGGTCGATGATGAGAACCTTATATTATCCTGAAGAGAGGTGATGACTTAAAAATCATGCTCAAT
AGGATTACGCTGAGGCCC
SEQ ID NO: 10
SNORD115-17 human genomic sequence
GGGTCGATGATGAGAACCTTATATTGTCCTGAAGAGAGGTGATGACTTAAAAATCATTCTCAAA
AGGATTATGCTGAGGCCC
SEQ ID NO: 11
SNORD115-18 human genomic sequence
GGGTCGATGATGAGAACCTTATATTGTCCTGAAGAGAGGTGATGACTTAAAAATCATTCTCAAA
AGGATTATGCTGAGGCCC
SEQ ID NO: 12
SNORD115-19 human genomic sequence
GGGTCGATGATGAGAACCTTATATTGTCCTGAAGAGAGGTGATGACTTAAAAATCATTCTCAAA
AGGATTATGCTGAGGCCC
SEQ ID NO: 13
SNORD115-20 human genomic sequence
GGGTCGATGATGAGAACCTTATATTGTCCTGAAGAGAGGTGATGACTTAAAAATCATGCTCAAT
AGGATTATGCTGAGGCCC
SEQ ID NO: 14
SNORD115-21 human genomic sequence
GGGTCGATGATGAGAACCTTATATTTTCTGAAGAGAGGTGATGACTTAAAAATCATGCTCAATA
GGATTACGCTGAGGCCC
SEQ ID NO: 15
SNORD115-27 human genomic sequence
GGGCTGATGATGAGAACCTTATATTGTCCTGAAAAGAGGTGATGACTTAACAATCATGCTCAAT
AGGATTACATTGAAGCCC
SEQ ID NO: 16
SNORD115-37 human genomic sequence
GGGCTGATGATGAGAACCTTATATTGTCCTGAAAAAAGGTGATGACTTAAACATCATGCTTAAT
AGTATTATGCTGAAGCCC
SEQ ID NO: 17
SNORD115-40 human genomic sequence
GGGTCGATGATGAGAACCTTATATTTTCCTGAAGAGAGGTGATGACTTAAAAATCATGCTCAAT
AGGATTACGCTGAGGCCC
SEQ ID NO: 18
SNORD115-42 human genomic sequence
GGGTCGATGATGAGAACCTTATATTGTTCTGAAGAGAGGTGATGACTTAAAAATCATGCTCAAT
AGGATTACGCTGAGGCCC
SEQ ID NOS: 19-360
SNORD115 artificial/synthetic sequences
  19. CAATGATGAGAACCGTATATT
  20. GGTAACCTGTTCTCCAAATTT
  21. TGATGATGAGAACCTTATATT
  22. GCCTTCACCTCTACCTATTAT
  23. CCTTCACCTCTACCTATTATA
  24. CTTCACCTCTACCTATTATAA
  25. TTCACCTCTACCTATTATAAT
  26. TCACCTCTACCTATTATAATA
  27. CACCTCTACCTATTATAATAT
  28. GATACAGCTTCCTTGAATATA
  29. ATACAGCTTCCTTGAATATAT
  30. TACAGCTTCCTTGAATATATA
  31. ACAGCTTCCTTGAATATATAA
  32. CAGCTTCCTTGAATATATAAA
  33. CTGTATGATTTGTCCTTATTA
  34. AGCACCCAGGTGTCCTTTAAT
  35. TTCTGATTTGGTGAGAAATAA
  36. TCTGATTTGGTGAGAAATAAT
  37. CTGTATCAGTGCACCTATAAA
  38. TGTATCAGTGCACCTATAAAT
  39. TTTGCTTCAGTGACCTTTAAT
  40. TTGCTTCAGTGACCTTTAATT
  41. TGCTTCAGTGACCTTTAATTT
  42. AGAGATGTGCCATTCTATTAT
  43. GAGATGTGCCATTCTATTATA
  44. AGATGTGCCATTCTATTATAA
  45. GATGTGCCATTCTATTATAAA
  46. TCCTTTACCGACGTGTATATT
  47. ACTGTTACCTAGTCCAATATT
  48. CTGTTACCTAGTCCAATATTT
  49. TGTTACCTAGTCCAATATTTA
  50. GTTACCTAGTCCAATATTTAA
  51. AGTATGTACTTTGCCTATAAA
  52. GCACATGCTCAGAAGAAATAA
  53. CACATGCTCAGAAGAAATAAA
  54. TTGAGGGCTTCAGTGTTTAAA
  55. TGGAGATCTTTAACCTTTAAT
  56. GACTCAGCTTTCAGCTTATTT
  57. TTTCTCTTGGCGTAGTAAATT
  58. GATGACACTTATTCCTAATAT
  59. ATGACTGGGTTCACAAATTTA
  60. AGAATCATGGATCTCATTATA
  61. GGTCTAGAGCCTTAGTAATAT
  62. AGGCATGCTGCAGTGAATTTA
  63. GGCATGCTGCAGTGAATTTAA
  64. TATCTTCAGGAGACGTAATAA
  65. ATCTTCAGGAGACGTAATAAT
  66. GAGAGAATATCTTGGTTTATT
  67. TGTGTCATTACACGGATTATA
  68. GTGTCATTACACGGATTATAT
  69. TGTCATTACACGGATTATATA
  70. GTCATTACACGGATTATATAT
  71. GAGTTCTGTCTTTCGTAATTA
  72. AGTTCTGTCTTTCGTAATTAA
  73. GTTCTGTCTTTCGTAATTAAA
  74. CAAGTGGACCATTTGAATATT
  75. AGTGGACCATTTGAATATTAA
  76. GTGGACCATTTGAATATTAAA
  77. CCTGAACGTACTTTGATTTAA
  78. CTGAACGTACTTTGATTTAAA
  79. GGTGTCATTAGATTCATTATA
  80. CTTAAGTAACGGGTCATATTT
  81. ATCTCAGTCTTGTAGAAATTA
  82. TTAGGAGTCATGCACATATAA
  83. CCATCTCTGGTAAGCATATTT
  84. GTTTCCTACAATTCCTAATTT
  85. GTGAAGACTGAGCTCTATAAT
  86. TGAAGACTGAGCTCTATAATA
  87. GAAGACTGAGCTCTATAATAA
  88. AGACTGAGCTCTATAATAATT
  89. GACTGAGCTCTATAATAATTA
  90. ACAGTTATCGTGGTCTAAATA
  91. CAGTTATCGTGGTCTAAATAT
  92. AGTTATCGTGGTCTAAATATA
  93. TGCCCATGAAACTTGAAATTA
  94. GAATAATGCCAATGGTAAATA
  95. GACCTTTATGGACACTTATAT
  96. CTACAAGAGGGAAACTTTATT
  97. TACAAGAGGGAAACTTTATTT
  98. GATGCTCATTTGGTCTAAATA
  99. ATGCTCATTTGGTCTAAATAT
100. TGCTCATTTGGTCTAAATATA
101. CATATCCCATCATTCATTAAA
102. GGCTCCAGTATGATCATTATA
103. GCTCCAGTATGATCATTATAT
104. GATCAATTGAAGGTCTTATTT
105. AGGTCTTATTTCTGCATTAAA
106. CCTGTATAGTTAGTCTTTATT
107. CATACATACAGGAGGTAATAT
108. ATACATACAGGAGGTAATATT
109. TACATACAGGAGGTAATATTT
110. GAACTTTCAACCAGGATTTAA
111. TGGCTGAAACCTCCCATATTT
112. AGCAAAGGCATAACATATTAA
113. GCAAAGGCATAACATATTAAA
114. GACTACAGATGGTAGATTAAT
115. ACTACAGATGGTAGATTAATA
116. CTACAGATGGTAGATTAATAT
117. TTGGCTTATAGCATCAATAAA
118. TGGCTTATAGCATCAATAAAT
119. TTTGGGTGAGTTAAGATATAT
120. GAGAAGAAGAATGACATAATA
121. TATCTCATTTCACCCATATAT
122. ATCTCATTTCACCCATATATA
123. TCTCATTTCACCCATATATAT
124. GGGTATGGATGGGACAAATAT
125. CCCTGAAATAACAGCATTAAT
126. ATTTGACCATTCCCAATTATA
127. TGGATGTAAGAACACTATTAA
128. ATAGCAGAATGCACATTATAT
129. TAGCAGAATGCACATTATATT
130. AGTGCTCATAAAGCATTATTT
131. AGAGTGCCTATGTAGTTAATA
132. GAGTGCCTATGTAGTTAATAT
133. AGTGCCTATGTAGTTAATATT
134. GTGCCTATGTAGTTAATATTT
135. ACCACAGGCTTCTCAATTATT
136. CCACAGGCTTCTCAATTATTA
137. CACAGGCTTCTCAATTATTAA
138. ACAGGCTTCTCAATTATTAAT
139. CAGGCTTCTCAATTATTAATT
140. TCCTCTAGAACTGCCTTTAAA
141. CCTCTAGAACTGCCTTTAAAT
142. ACTGCCTTTAAATGCATATTA
143. TCAGGAGTTAATAGGATATTT
144. ACAACACAGATCTTCTATTAT
145. ACGGCACACCAGGAGATTATA
146. CGGCACACCAGGAGATTATAT
147. AGACAGGCTGCCTCCTTAAAT
148. CATTTGCCGTTCTGCAATATT
149. ATTTGCCGTTCTGCAATATTT
150. GCTTCAGATGATCGCTAATAA
151. TGAGTGGCTAACTAGAATAAA
152. AGCATAGAGAAGACCTTAAAT
153. GTAACTCCAAGACACATAATT
154. GCCAAACTAAGCTTCATAATT
155. TGGGCTAAATTCTCCAATTAA
156. GGGCTAAATTCTCCAATTAAA
157. ACACACATAGGCTCCAAATAA
158. CACACATAGGCTCCAAATAAA
159. TCTGATGAAACAGACTTTAAA
160. AGAGCTAACTATCCTAAATAT
161. GAGCTAACTATCCTAAATATA
162. CTTAGACTCCCACACAATAAT
163. TTAGACTCCCACACAATAATA
164. TAGACTCCCACACAATAATAA
165. AGACTCCCACACAATAATAAT
166. ATGAAATGAAGGCAGAAATAA
167. TGAAATGAAGGCAGAAATAAA
168. CAGAATCTCTGGGACATATTT
169. AGAATCTCTGGGACATATTTA
170. GAATCTCTGGGACATATTTAA
171. GCAGTGTGTAGAGAGAAATTT
172. CAGTGTGTAGAGAGAAATTTA
173. AGTGTGTAGAGAGAAATTTAT
174. GTGTGTAGAGAGAAATTTATA
175. ACACCCTAACATCACAATTAA
176. CACCCTAACATCACAATTAAA
177. CTAGCAGAAGGCAGGAAATAA
178. GACCACTAGCAAGACTAATAA
179. ACCACTAGCAAGACTAATAAA
180. GAATCCAATAGATGCAATAAA
181. ACCATCAGAGAATACTATAAA
182. CTCTGAAATTGAGGCAATAAT
183. TCTGAAATTGAGGCAATAATT
184. CTGAAATTGAGGCAATAATTA
185. TCAGGCAGGAGAAAGAAATAA
186. CAGGCAGGAGAAAGAAATAAA
187. CAGATGACATGATTGTATATT
188. ACCACTGCTCAACGAAATAAA
189. CCATACTGCCCAAGGTAATTT
190. CATACTGCCCAAGGTAATTTA
191. ATACTGCCCAAGGTAATTTAT
192. TACTGCCCAAGGTAATTTATA
193. GGAAAGGATTCCCTATTTAAT
194. GAAAGGATTCCCTATTTAATA
195. GTTAGAATGGCAATCATTAAA
196. GTACATACCCAAAGGATTATA
197. TACATACCCAAAGGATTATAT
198. ACATACCCAAAGGATTATATA
199. CATACCCAAAGGATTATATAT
200. CACGAAGTAGGACAGTATAAT
201. ACGAAGTAGGACAGTATAATT
202. CGAAGTAGGACAGTATAATTT
203. ACCACCATGCCTGGCTAATTT
204. TTAAGAACAAGACCCAAATAT
205. TAAGAACAAGACCCAAATATA
206. CTATGAGAAATGCACTTTAAA
207. ATATCCCATGCTAACATTAAT
208. GACTTTAGAACAAGGAATATT
209. ATGCACCTCAACAAATTTAAA
210. CACAGAAGTCTCAAGATAAAT
211. ACAGAAGTCTCAAGATAAATT
212. CAGAAGTCTCAAGATAAATTT
213. GCAGTGCCTACTGGGAAATTT
214. CAGTGCCTACTGGGAAATTTA
215. AGTGCCTACTGGGAAATTTAT
216. GTGCCTACTGGGAAATTTATA
217. TGCTGCACCCAGGGCAATTTA
218. GCTGCACCCAGGGCAATTTAT
219. CTGCACCCAGGGCAATTTATA
220. CTCTGATAAGCATGCTAATAT
221. GGCAATATGGAGATGTTATAT
222. AGATGCAATCATCCCATTAAT
223. ATTCTCTCTTAAGGCTATTAT
224. TGTTCACTTGGGTACATATAT
225. GTTCACTTGGGTACATATATA
226. CATGCCAGTGGACACAAATAT
227. TATGGCTTCCTCATGATTAAA
228. GCTACCATTCCTGGCTAATTT
229. TGCTGGCACAAGAACTTTATA
230. CCACATACCCTAAAGAATTAT
231. CACATACCCTAAAGAATTATT
232. CTTACCATCCGTTTCATTTAA
233. TTACCATCCGTTTCATTTAAA
234. TTTAGGACCTTAAGGATAAAT
235. TTAGGACCTTAAGGATAAATT
236. CTCACCAACTGGATGATTTAA
237. TCACCAACTGGATGATTTAAA
238. CACCAACTGGATGATTTAAAT
239. ACCAACTGGATGATTTAAATT
240. TCCCACTGATTAGTCAATATT
241. CCCACTGATTAGTCAATATTA
242. GATACTGATGAGAAGAAATTT
243. TCCTGAGTAGCTGGGATTATA
244. ACCACCATGCCTGGCTAATTT
245. GCCCAACTCTAGATCTTAAAT
246. CCCAACTCTAGATCTTAAATA
247. CATACCTGGCCAGAGATTATT
248. ATACCTGGCCAGAGATTATTA
249. ACCTGGCCAGAGATTATTAAA
250. CCTGGCCAGAGATTATTAAAT
251. CTGGCCAGAGATTATTAAATA
252. GAATTGGACATCTGGTAATAA
253. GTACTGAAGTACAAGATTATA
254. ACTGTCTATGTAGAGATTAAA
255. CTGTCTATGTAGAGATTAAAT
256. TGGCATTGCAGATTGAAATTT
257. TCAATTTCTAAGCCCATTATA
258. CAATTTCTAAGCCCATTATAT
259. TGGTATGACAGTTTCAAATAA
260. ACCCACTGAACTAGGAAATAT
261. CCCACTGAACTAGGAAATATT
262. AGACTAACATCTCTGTAATAT
263. GACTAACATCTCTGTAATATA
264. TGCGGAAAGGCAACCATAAAT
265. GCGGAAAGGCAACCATAAATA
266. CGGAAAGGCAACCATAAATAT
267. GGAAAGGCAACCATAAATATA
268. TTGTATTGCACACCCATTAAA
269. TGGTGTGCAGGCAGGTTTATT
270. TGGGAGACTAACTACTAATTA
271. GGGAGACTAACTACTAATTAA
272. TTCAAGTGTCAACTGTATATA
273. TCAAGTGTCAACTGTATATAT
274. TATGATTAGCAGCTGAATTTA
275. ATGATTAGCAGCTGAATTTAA
276. TAGAAATGAGCTACGTAATTT
277. GCAAAGAATGAGCAGTTAATA
278. TTTCATGTACTTCCGTATATT
279. TTACTTCTCTGAGGGATTTAT
280. TACTTCTCTGAGGGATTTATT
281. CTGTCCAGGACATCCATTAAA
282. TGTCCAGGACATCCATTAAAT
283. GTCCAGGACATCCATTAAATT
284. TAAGAGCTGACGCCCAAATTA
285. TTGATGATGAGAACCTTATAT
286. GATGATGAGAACCTTATATTA
287. CATGCTTCACAAGGCAATAAA
288. TCGGGAGACCAGGGCTTATTT
289. TGACGGGATTAAGAGATTAAA
290. AGACAGGCATAGGAAATAATA
291. GACAGGCATAGGAAATAATAA
292. TTGATTTGGGAAGTGATAAAT
293. TCAATGATGAGAACCTTATAT
294. CAATGATGAGAACCTTATATT
295. GTCGATGATGAGAACTTTATA
296. TCGATGATGAGAACTTTATAT
297. CGATGATGAGAACTTTATATT
298. TCGATGATGAGAACCTTATAT
299. CGATGATGAGAACCTTATATT
300. CCCAGCATGGCCAGTATATTT
301. CAAGAATCGCACACGAATTAT
302. GGGTCAATGAGAACCTTATAT
303. GGTCAATGAGAACCTTATATT
304. CAATGGGACCGACCCTATTAA
305. GAATGGGACCGACCCTATTAA
306. AGGGCAGCTTCCTTGTTAATT
307. AGTTGAGTTGCAGAGATTTAA
308. ATTTCTGGTGTGTGCTTATAA
309. TTTCTGGTGTGTGCTTATAAA
310. TGCCTGGATGGGTTCATTTAA
311. GCCTGGATGGGTTCATTTAAT
312. GGAGTTGAGCTGCAGATATTT
313. GAGTTGAGCTGCAGATATTTA
314. AGTTGAGCTGCAGATATTTAA
315. TAAGGATCACACAGGATTTAT
316. TCGATGATGAGAAACTTATAT
317. CGATGATGAGAAACTTATATT
318. GAAGAGGATGCTGGGAATTAT
319. GAATGGGACAAACCCTAATAA
320. ATTTCTGGTGTGAGCTTATAA
321. TTTCTGGTGTGAGCTTATAAA
322. TGGTCCTGTCCCAGGAATTTA
323. AGTCGAGCTGTGGAGATTTAA
324. CCTGGCAGGGCCACTATAATT
325. CTGGCAGGGCCACTATAATTT
326. GATGATGAGAACCTTATATTT
327. CAATGATGAGAACCCTATATT
328. CCCAACCTAGGTGAGAAATTT
329. TCAGTGTCGAGAACCTTATAT
330. CAGTGTCGAGAACCTTATATT
331. CCTGGCAGGGCCCTTATATTT
332. TCGATTATGAGAACCTTATAT
333. CGATTATGAGAACCTTATATT
334. TCAGTGATGAGAACCTTATAT
335. CAGTGATGAGAACCTTATATT
336. CTGATGATGAGAACCTTATAT
337. CCAGCTAGACCACACTTATTT
338. AGGACGTTGGGATCCTATTAT
339. TTCTGAAGAGAGGTGATTATT
340. TCTGAAGAGAGGTGATTATTT
341. CTGAAGAGAGGTGATTATTTA
342. TGAAGAGAGGTGATTATTTAA
343. GAAGAGAGGTGATTATTTAAA
344. GGTTGGATTGTGGAGATTAAA
345. GGCCCAGCCTAGGTGATAATT
346. GCCCAGCCTAGGTGATAATTT
347. CGTTGGGTCTCAGAGATTTAA
348. CCCACCCTAAGTGAGAAATTT
349. TCAATGATGAGAACCTTATAA
350. CAATGATGAGAACCTTATAAT
351. ATGATGAGAACCTTGTATTAT
352. CCCAACAGGGCCACCATATTT
353. CATTGGTGACAAGTCAATATT
354. ATTGGTGACAAGTCAATATTT
355. TGCAGTCCCAGAATGTAATTT
356. CCAGGCAGGGCCACTATATTT
357. CCTGGCAGGGCCACTATATTT
358. TTGCGGTCATGGGCCATAAAT
359. AGCAGCTCCCAGCTGAAATTT
360. AGGGACCTGTCCTGGAAATTT
SEQ ID NO: 361
shRNA artificial/synthetic sequences
5′-TGATGATGAGAACCTTATATTnnnnnnnnAATATAAGGTTCTCATCATCA-3′,
wherein
nnnnnnnn can be CTCGAG (SEQ ID NO: 362), TCAAGAG (SEQ ID
NO: 363), TTCG (SEQ ID NO: 364) or GAAGCTTG (SEQ ID NO: 365)
SEQ ID NO: 362
stem loop artificial/synthetic sequences
CTCGAG
SEQ ID NO: 363
stem loop artificial/synthetic sequences
TCAAGAG
SEQ ID NO: 364
stem loop artificial/synthetic sequences
TTCG
SEQ ID NO: 365
stem loop
GAAGCTTG
SEQ ID NO: 366
shRNA artificial/synthetic sequences
GATGATGAGAACCTTATATTCTCGAGAATATAAGGTTCTCATCATC
SEQ ID NO: 367
shRNA artificial/synthetic sequences
ATGATGAGAACCTTATATTCTCGAGAATATAAGGTTCTCATCAT
SEQ ID NO: 368
shRNA artificial/synthetic sequences
TGATGAGAACCTTATATTCTCGAGAATATAAGGTTCTCATCA
SEQ ID NO: 369
shRNA artificial/synthetic sequences
GATGAGAACCTTATATTCTCGAGAATATAAGGTTCTCATC
SEQ ID NO: 370
shRNA artificial/synthetic sequences
TGATGATGAGAACCTTATATCTCGAGATATAAGGTTCTCATCATCA
SEQ ID NO: 371
shRNA artificial/synthetic sequences
TGATGATGAGAACCTTATACTCGAGTATAAGGTTCTCATCATCA
SEQ ID NO: 372
shRNA artificial/synthetic sequences
TGATGATGAGAACCTTATCTCGAGATAAGGTTCTCATCATCA
SEQ ID NO: 373
shRNA artificial/synthetic sequences
TGATGATGAGAACCTTACTCGAGTAAGGTTCTCATCATCA
SEQ ID NO: 374
shRNA artificial/synthetic sequences
GATGATGAGAACCTTATATTnnnnnnnnAATATAAGGTTCTCATCATC, wherein
nnnnnnnn can be CTCGAG (SEQ ID NO: 362), TCAAGAG (SEQ ID NO:
363), TTCG (SEQ ID NO: 364) or GAAGCTTG (SEQ ID NO: 365).
SEQ ID NO: 375
shRNA artificial/synthetic sequences
ATGATGAGAACCTTATATTnnnnnnnnAATATAAGGTTCTCATCAT, wherein
nnnnnnnn can be CTCGAG (SEQ ID NO: 362), TCAAGAG (SEQ ID NO:
363), TTCG (SEQ ID NO: 364) or GAAGCTTG (SEQ ID NO: 365).
SEQ ID NO: 376
shRNA artificial/synthetic sequences
TGATGAGAACCTTATATTnnnnnnnnAATATAAGGTTCTCATCA, wherein nnnnnnnn
can be CTCGAG (SEQ ID NO: 362), TCAAGAG (SEQ ID NO: 363), TTCG
(SEQ ID NO: 364) or GAAGCTTG (SEQ ID NO: 365).
SEQ ID NO: 377
shRNA artificial/synthetic sequences
GATGAGAACCTTATATTnnnnnnnnAATATAAGGTTCTCATC, wherein nnnnnnnn can
be CTCGAG (SEQ ID NO: 362), TCAAGAG (SEQ ID NO: 363), TTCG (SEQ
ID NO: 364) or GAAGCTTG (SEQ ID NO: 365).
SEQ ID NO: 378
shRNA artificial/synthetic sequences
TGATGATGAGAACCTTATATnnnnnnnnATATAAGGTTCTCATCATCA, wherein
nnnnnnnn can be CTCGAG (SEQ ID NO: 362), TCAAGAG (SEQ ID NO:
363), TTCG (SEQ ID NO: 364) or GAAGCTTG (SEQ ID NO: 365).
SEQ ID NO: 379
shRNA artificial/synthetic sequences
TGATGATGAGAACCTTATAnnnnnnnnTATAAGGTTCTCATCATCA, wherein nnnnnnnn
can be CTCGAG (SEQ ID NO: 362), TCAAGAG (SEQ ID NO: 363), TTCG
(SEQ ID NO: 364) or GAAGCTTG (SEQ ID NO: 365).
SEQ ID NO: 380
shRNA artificial/synthetic sequences
TGATGATGAGAACCTTATnnnnnnnnATAAGGTTCTCATCATCA, wherein nnnnnnnn
can be CTCGAG (SEQ ID NO: 362), TCAAGAG (SEQ ID NO: 363), TTCG
(SEQ ID NO: 364) or GAAGCTTG (SEQ ID NO: 365).
SEQ ID NO: 381
shRNA artificial/synthetic sequences
TGATGATGAGAACCTTAnnnnnnnnTAAGGTTCTCATCATCA, wherein nnnnnnnn can
be CTCGAG (SEQ ID NO: 362), TCAAGAG (SEQ ID NO: 363), TTCG (SEQ
ID NO: 364) or GAAGCTTG (SEQ ID NO: 365).
SEQ ID NO: 382
shRNA artificial/synthetic sequences
CAATGATGAGAACCGTATATTCTCGAGAATATACGGTTCTCATCATTG
SEQ ID NO: 383
shRNA artificial/synthetic sequences
GGTAACCTGTTCTCCAAATTTCTCGAGAAATTTGGAGAACAGGTTACC
```

What is claimed is:

1. A polynucleotide sequence comprising:
(SEQ ID NO: 3)
5′-TGATGATGAGAACCTTATATTCTCGAGAATATAAGGTTCTCATCAT
CA-3′.

2. An expression vector comprising the polynucleotide sequence of claim 1.

3. The expression vector according to claim 2, further comprising a promoter.

4. The expression vector according to claim 3, wherein the promotor is a neuron specific promoter.

5. The expression vector according to claim 4, wherein neuron specific promoter is neuron-specific enolase (NSE), synapsin I (Syn), or Ca2+/CaM-activated protein kinase II alpha (CaMKIIalpha).

6. The expression vector according to claim 3, wherein the promotor is a U6 promoter or a H1 promoter.

7. The expression vector according to claim 2, wherein the expression vector is an adeno-associated viral (AAV) vector or a lentiviral vector.

8. The expression vector according to claim 7, wherein the expression vector is AAV1, AAV2, AAV3, AAVS, AAV6, AAV7, AAV8, AAV9, or AAV10.

9. A pharmaceutical composition comprising the polynucleotide sequence according to claim 1 and a pharmaceutically acceptable carrier.

10. The pharmaceutical composition according to claim 9, wherein the polynucleotide sequence is contained within an expression vector.

11. The pharmaceutical composition according to claim 10, wherein the expression vector is an AAV vector or a lentivirus vector.

12. A polynucleotide encoding a shRNA comprising a nucleotide sequence that is at least 85%, at least 90%, at least 95%, or 100% complementary to a RNA encoded by any of SEQ ID NOs: 19-360.

13. The polynucleotide of claim 12, wherein the polynucleotide is SEQ ID NO: 3.

14. The polynucleotide of claim 12, wherein the shRNA causes activation of, or an increase in, expression of paternal UBE3A.

15. The polynucleotide of claim 12, wherein the shRNA causes a reduction of expression of paternal UBE3A-ATS.

16. The polynucleotide of claim 12, wherein the shRNA causes a reduction of expression of paternal SNORD115.

17. An expression vector comprising the polynucleotide of claim 12 and a promoter.

18. The expression vector of claim 17, wherein the promoter is a neuron specific promoter.

19. The expression vector according to claim 18, wherein neuron specific promoter is neuron-specific enolase (NSE), synapsin I (Syn), or Ca2+/CaM-activated protein kinase II alpha (CaMKIIalpha).

20. The expression vector of claim 17, wherein the promoter is a U6 promoter or a H1 promoter.

21. The expression vector according to claim 17, wherein the expression vector is an adeno-associated viral (AAV) vector or a lentiviral vector.

22. The expression vector according to claim 21, wherein the expression vector is AAV1, AAV2, AAV3, AAVS, AAV6, AAV7, AAV8, AAV9, or AAV10.

23. The expression vector of claim 17, wherein the polynucleotide is a DNA polynucleotide.

24. A pharmaceutical composition comprising the polynucleotide sequence according to claim 12 and a pharmaceutically acceptable carrier.

25. The pharmaceutical composition according to claim 24, wherein the polynucleotide sequence is contained within an expression vector.

26. The pharmaceutical composition according to claim 25, wherein the expression vector is an AAV vector or a lentivirus vector.

27. A method of treating Angelman syndrome comprising administering to a patient in need thereof the polynucleotide according to claim 1.

28. The method of treating Angelman syndrome according to claim 27, wherein the polynucleotide encodes a shRNA which causes a reduction of expression of paternal UBE3A-ATS.

29. The method of treating Angelman syndrome according to claim 27, wherein the polynucleotide encodes a shRNA which causes a reduction of expression of paternal SNORD 115.

30. The method of treating Angelman syndrome according to claim 27, wherein the polynucleotide encodes a shRNA which causes activation of, or an increase in, expression of paternal UBE3A gene.

31. A method of treating Angelman syndrome comprising administering to a patient in need thereof the polynucleotide according to claim 12.

32. The method of treating Angelman syndrome according to claim 31, wherein the polynucleotide encodes a shRNA which causes a reduction of expression of paternal UBE3A-ATS.

33. The method of treating Angelman syndrome according to claim 31, wherein the polynucleotide encodes a shRNA which causes a reduction of expression of paternal SNORD 115.

34. The method of treating Angelman syndrome according to claim 31, wherein the polynucleotide encodes a shRNA which causes activation of, or an increase in, expression of paternal UBE3A gene.

35. A polynucleotide comprising SEQ ID NO: 3 encoding a shRNA wherein the shRNA is capable of inhibiting the silencing of paternal UBE3A.

36. A method of inhibiting the silencing of a paternal UBE3A gene by an RNA antisense transcript encoded by SEQ ID NO: 2 comprising administering to a patient in need thereof, an amount of the polynucleotide of claim 1 encoding a shRNA effective to cut the RNA antisense transcript encoded by SEQ ID NO: 2.

37. The method of claim 36, wherein the polynucleotide is contained within an expression vector.

38. The method of claim 37, wherein the expression vector is a AAV vector or a lentivirus vector.

39. The method of claim 36, wherein the polynucleotide is administered to the patient's brain.

40. The method of claim 36, wherein the polynucleotide is administered to neurons of the patient.

41. The method of claim 33, wherein the shRNA reduces or terminates transcription of a polynucleotide comprising the sequence of SEQ ID NO: 1.

42. The method of claim 36, wherein the shRNA reduces the levels of the RNA antisense transcript encoded by SEQ ID NO: 1.

43. A method of inhibiting the silencing of paternal UBE3A gene by the RNA antisense transcript encoded by SEQ ID NO: 1 comprising administering to a patient in need thereof, an amount of the polynucleotide of claim 12 encoding a shRNA effective to cut the RNA antisense transcript encoded by SEQ ID NO: 2.

44. The method of claim 43, wherein the polynucleotide is contained within an expression vector.

45. The method of claim 44, wherein the expression vector is a AAV vector or a lentivirus vector.

46. The method of claim 43, wherein the polynucleotide is administered to the patient's brain.

47. The method of claim 43, wherein the polynucleotide is administered to neurons of the patient.

48. The method of claim 43, wherein the shRNA reduces or terminates transcription of a polynucleotide comprising the sequence of SEQ ID NO: 1.

49. The method of claim 43, wherein the shRNA reduces the levels of the RNA antisense transcript encoded by SEQ ID NO: 1.

50. The polynucleotide of claim 1, for use in treating Angelman syndrome, for use in activating paternal UBE3A, or for use in inhibiting the silencing of paternal UBE3A gene by the RNA antisense transcript encoded by SEQ ID NO: 1.

51. The polynucleotide of claim 12, for use in treating Angelman syndrome, for use in activating paternal UBE3A, or for use in inhibiting the silencing of paternal UBE3A gene by the RNA antisense transcript encoded by SEQ ID NO: 1.

52. Use of the polynucleotide of claim 1, in the manufacture of a medicament for the treatment of Angelman syndrome, for activation of paternal UBE3A, or for inhibition of the silencing of paternal UBE3A gene by the RNA antisense transcript encoded by SEQ ID NO: 1.

53. Use of the polynucleotide of claim 12, in the manufacture of a medicament for the treatment of Angelman syndrome, for activation of paternal UBE3A, or for inhibition of the silencing of paternal UBE3A gene by the RNA antisense transcript encoded by SEQ ID NO: 1.

54. A shRNA encoded by a portion of SEQ ID NO: 3, wherein the portion of SEQ ID NO: 3 defines a first segment defined by the bold nucleotides which has been shortened by one, two, three or four nucleotides at either end of the first segment, and a second segment defined by the italicized nucleotides which has been shortened by one, two or three nucleotides at either end of the italicized nucleotides.

55. The shRNA of claim 54, wherein the shRNA is encoded by SEQ ID NO: 366, SEQ ID NO: 367, SEQ ID NO: 368, SEQ ID NO: 369, SEQ ID NO: 370, SEQ ID NO: 371, SEQ ID NO: 372 or SEQ ID NO: 373.

56. A polynucleotide sequence comprising:
(SEQ ID NO: 361)
5′-TGATGATGAGAACCTTATATTnnnnnnnnAATATAAGGTTCTCATC
ATCA-3′, wherein nnnnnnnn can be
(SEQ ID NO: 362)
CTCGAG,
(SEQ ID NO: 363)
TCAAGAG,
(SEQ ID NO: 364)
TTCG
or
(SEQ ID NO: 365)
GAAGCTTG.

57. A polynucleotide sequence comprising a first portion, a second portion and a third portion, the first portion comprising any of SEQ ID NOs: 19-360, the second portion comprising any of SEQ ID Nos: 362, 363, 364, or 365, and the third portion comprising respective nucleotide sequences complementary to those in SEQ ID NOs: 19-360.

58. The polynucleotide of claim 57 wherein the first portion is shortened by one, two, three or four nucleotides at either end of the first portion, and the third portion is shortened by one, two or three nucleotides at either end of the third portion.
